SEQUENCE LISTING (405 sequences)

SEQ ID NO: 1 - RNA, 21 nt, synthetic construct
  source 1..21: mol_type = other RNA
gttggtcacttctgtgcaaat

SEQ ID NO: 2 - RNA, 21 nt, synthetic construct
  source 1..21: mol_type = other RNA
ttgcacagaagtgaccaaccc

SEQ ID NO: 3 - RNA, 21 nt, synthetic construct
  source 1..21: mol_type = other RNA
gggattcagaaatgtggatta

SEQ ID NO: 4 - RNA, 21 nt, synthetic construct
  source 1..21: mol_type = other RNA
atccacatttctgaatcccag

SEQ ID NO: 5 - RNA, 21 nt, synthetic construct
  source 1..21: mol_type = other RNA
gttcaccaattgattcttaaa

SEQ ID NO: 6 - RNA, 21 nt, synthetic construct
  source 1..21: mol_type = other RNA
taagaatcaattggtgaacac

SEQ ID NO: 7 - RNA, 21 nt, synthetic construct
  source 1..21: mol_type = other RNA
gaccctcagtggttttagatc

SEQ ID NO: 8 - RNA, 21 nt, synthetic construct
  source 1..21: mol_type = other RNA
tctaaaaccactgagggtcca

SEQ ID NO: 9 - RNA, 21 nt, synthetic construct
  source 1..21: mol_type = other RNA
gggccaaatacctatgtaata

SEQ ID NO: 10 - RNA, 21 nt, synthetic construct
  source 1..21: mol_type = other RNA
ttacataggtatttggccctg

SEQ ID NO: 11 - RNA, 21 nt, synthetic construct
  source 1..21: mol_type = other RNA
gctcttcaatataaaggctcg

SEQ ID NO: 12 - RNA, 21 nt, synthetic construct
  source 1..21: mol_type = other RNA
agcctttatattgaagagcca

SEQ ID NO: 13 - RNA, 21 nt, synthetic construct
  source 1..21: mol_type = other RNA
gagcagcctatgaaggagaag

SEQ ID NO: 14 - RNA, 21 nt, synthetic construct
  source 1..21: mol_type = other RNA
tctccttcataggctgctcgg

SEQ ID NO: 15 - RNA, 21 nt, synthetic construct
  source 1..21: mol_type = other RNA
gagtggcaatgtcaggtgtta

SEQ ID NO: 16 - RNA, 21 nt, synthetic construct
  source 1..21: mol_type = other RNA
acacctgacattgccactcat

SEQ ID NO: 17 - RNA, 21 nt, synthetic construct
  source 1..21: mol_type = other RNA
gctcaccattccaatatctca

SEQ ID NO: 18 - RNA, 21 nt, synthetic construct
  source 1..21: mol_type = other RNA
agatattggaatggtgagcct

SEQ ID NO: 19 - RNA, 21 nt, synthetic construct
  source 1..21: mol_type = other RNA
gaactacatgtcacagtattt

SEQ ID NO: 20 - RNA, 21 nt, synthetic construct
  source 1..21: mol_type = other RNA
atactgtgacatgtagttctt

SEQ ID NO: 21 - RNA, 21 nt, synthetic construct
  source 1..21: mol_type = other RNA
gtgcctctgttgatcgtcttg

SEQ ID NO: 22 - RNA, 21 nt, synthetic construct
  source 1..21: mol_type = other RNA
agacgatcaacagaggcacat

SEQ ID NO: 23 - RNA, 21 nt, synthetic construct
  source 1..21: mol_type = other RNA
gtgccacagttggcatagatg

SEQ ID NO: 24 - RNA, 21 nt, synthetic construct
  source 1..21: mol_type = other RNA
tctatgccaactgtggcactt

SEQ ID NO: 25 - RNA, 21 nt, synthetic construct
  source 1..21: mol_type = other RNA
gttttgcaaaagtagctatgg

SEQ ID NO: 26 - RNA, 21 nt, synthetic construct
  source 1..21: mol_type = other RNA
atagctacttttgcaaaacac

SEQ ID NO: 27 - RNA, 21 nt, synthetic construct
  source 1..21: mol_type = other RNA
gggttgctggagattgatatt

SEQ ID NO: 28 - RNA, 21 nt, synthetic construct
  source 1..21: mol_type = other RNA
tatcaatctccagcaacccag

SEQ ID NO: 29 - RNA, 21 nt, synthetic construct
  source 1..21: mol_type = other RNA
gacctgcctagaaatattatg

SEQ ID NO: 30 - RNA, 21 nt, synthetic construct
  source 1..21: mol_type = other RNA
taatatttctaggcaggtcag

SEQ ID NO: 31 - RNA, 21 nt, synthetic construct
  source 1..21: mol_type = other RNA
gacttgcacagtaataaattt

SEQ ID NO: 32 - RNA, 21 nt, synthetic construct
  source 1..21: mol_type = other RNA
atttattactgtgcaagtcca

SEQ ID NO: 33 - RNA, 21 nt, synthetic construct
  source 1..21: mol_type = other RNA
gttgctcaccaaatttgcaaa

SEQ ID NO: 34 - RNA, 21 nt, synthetic construct
  source 1..21: mol_type = other RNA
tgcaaatttggtgagcaacgc

SEQ ID NO: 35 - RNA, 21 nt, synthetic construct
  source 1..21: mol_type = other RNA
ggccctgaccagtattcatta

SEQ ID NO: 36 - RNA, 21 nt, synthetic construct
  source 1..21: mol_type = other RNA
atgaatactggtcagggccaa

SEQ ID NO: 37 - RNA, 21 nt, synthetic construct
  source 1..21: mol_type = other RNA
ggtcatcaataccgaagatgg

SEQ ID NO: 38 - RNA, 21 nt, synthetic construct
  source 1..21: mol_type = other RNA
atcttcggtattgatgaccag

SEQ ID NO: 39 - RNA, 21 nt, synthetic construct
  source 1..21: mol_type = other RNA
gagcttcaccagaatgcactc

SEQ ID NO: 40 - RNA, 21 nt, synthetic construct
  source 1..21: mol_type = other RNA
gtgcattctggtgaagctcca

SEQ ID NO: 41 - RNA, 21 nt, synthetic construct
  source 1..21: mol_type = other RNA
gtggcaatgtcaggtgttata

SEQ ID NO: 42 - RNA, 21 nt, synthetic construct
  source 1..21: mol_type = other RNA
taacacctgacattgccactc

SEQ ID NO: 43 - RNA, 21 nt, synthetic construct
  source 1..21: mol_type = other RNA
gcttgtcctaaagtggagagt

SEQ ID NO: 44 - RNA, 21 nt, synthetic construct
  source 1..21: mol_type = other RNA
tctccactttaggacaagcct

SEQ ID NO: 45 - RNA, 21 nt, synthetic construct
  source 1..21: mol_type = other RNA
ggcaatgtcaggtgttataat

SEQ ID NO: 46 - RNA, 21 nt, synthetic construct
  source 1..21: mol_type = other RNA
tataacacctgacattgccac

SEQ ID NO: 47 - RNA, 21 nt, synthetic construct
  source 1..21: mol_type = other RNA
ggtaggatctgaagtcttaga

SEQ ID NO: 48 - RNA, 21 nt, synthetic construct
  source 1..21: mol_type = other RNA
taagacttcagatcctaccag

SEQ ID NO: 49 - RNA, 21 nt, synthetic construct
  source 1..21: mol_type = other RNA
ggaaatgtcagtactccattg

SEQ ID NO: 50 - RNA, 21 nt, synthetic construct
  source 1..21: mol_type = other RNA
atggagtactgacatttccta

SEQ ID NO: 51 - RNA, 21 nt, synthetic construct
  source 1..21: mol_type = other RNA
gccttattttccaatgggatt

SEQ ID NO: 52 - RNA, 21 nt, synthetic construct
  source 1..21: mol_type = other RNA
tcccattggaaaataaggcat

SEQ ID NO: 53 - RNA, 21 nt, synthetic construct
  source 1..21: mol_type = other RNA
gattcactcaggtcatcaaaa

SEQ ID NO: 54 - RNA, 21 nt, synthetic construct
  source 1..21: mol_type = other RNA
ttgatgacctgagtgaatcat

SEQ ID NO: 55 - RNA, 21 nt, synthetic construct
  source 1..21: mol_type = other RNA
ggaactcttatttagccataa

SEQ ID NO: 56 - RNA, 21 nt, synthetic construct
  source 1..21: mol_type = other RNA
atggctaaataagagttccct

SEQ ID NO: 57 - RNA, 21 nt, synthetic construct
  source 1..21: mol_type = other RNA
gttgtccaaaacaccctaagg

SEQ ID NO: 58 - RNA, 21 nt, synthetic construct
  source 1..21: mol_type = other RNA
ttagggtgttttggacaacct

SEQ ID NO: 59 - RNA, 21 nt, synthetic construct
  source 1..21: mol_type = other RNA
gcagatcctacatcattcaga

SEQ ID NO: 60 - RNA, 21 nt, synthetic construct
  source 1..21: mol_type = other RNA
tgaatgatgtaggatctgcag

SEQ ID NO: 61 - RNA, 21 nt, synthetic construct
  source 1..21: mol_type = other RNA
gtttaagtcttataggatact

SEQ ID NO: 62 - RNA, 21 nt, synthetic construct
  source 1..21: mol_type = other RNA
tatcctataagacttaaaccc

SEQ ID NO: 63 - RNA, 21 nt, synthetic construct
  source 1..21: mol_type = other RNA
gagagcgtgtgatcagaataa

SEQ ID NO: 64 - RNA, 21 nt, synthetic construct
  source 1..21: mol_type = other RNA
attctgatcacacgctctctc

SEQ ID NO: 65 - RNA, 21 nt, synthetic construct
  source 1..21: mol_type = other RNA
gcagatgccaatcaagcaaag

SEQ ID NO: 66 - RNA, 21 nt, synthetic construct
  source 1..21: mol_type = other RNA
ttgcttgattggcatctgctc

SEQ ID NO: 67 - RNA, 21 nt, synthetic construct
  source 1..21: mol_type = other RNA
gccataatcagatcagcatct

SEQ ID NO: 68 - RNA, 21 nt, synthetic construct
  source 1..21: mol_type = other RNA
atgctgatctgattatggcta

SEQ ID NO: 69 - RNA, 21 nt, synthetic construct
  source 1..21: mol_type = other RNA
gatatgagcagcaatgatatt

SEQ ID NO: 70 - RNA, 21 nt, synthetic construct
  source 1..21: mol_type = other RNA
tatcattgctgctcatatcta

SEQ ID NO: 71 - RNA, 21 nt, synthetic construct
  source 1..21: mol_type = other RNA
gctggtccaaatcctggagga

SEQ ID NO: 72 - RNA, 21 nt, synthetic construct
  source 1..21: mol_type = other RNA
ctccaggatttggaccagcgt

SEQ ID NO: 73 - RNA, 21 nt, synthetic construct
  source 1..21: mol_type = other RNA
gctctcatttcttgacttaaa

SEQ ID NO: 74 - RNA, 21 nt, synthetic construct
  source 1..21: mol_type = other RNA
taagtcaagaaatgagagctg

SEQ ID NO: 75 - RNA, 21 nt, synthetic construct
  source 1..21: mol_type = other RNA
gaccttagatctcctcctaac

SEQ ID NO: 76 - RNA, 21 nt, synthetic construct
  source 1..21: mol_type = other RNA
taggaggagatctaaggtctt

SEQ ID NO: 77 - RNA, 21 nt, synthetic construct
  source 1..21: mol_type = other RNA
ggtcctttatttccagataag

SEQ ID NO: 78 - RNA, 21 nt, synthetic construct
  source 1..21: mol_type = other RNA
tatctggaaataaaggaccaa

SEQ ID NO: 79 - RNA, 21 nt, synthetic construct
  source 1..21: mol_type = other RNA
gagctttccacaacagctatg

SEQ ID NO: 80 - RNA, 21 nt, synthetic construct
  source 1..21: mol_type = other RNA
tagctgttgtggaaagctcgt

SEQ ID NO: 81 - RNA, 21 nt, synthetic construct
  source 1..21: mol_type = other RNA
ggcctatccaaataagagaca

SEQ ID NO: 82 - RNA, 21 nt, synthetic construct
  source 1..21: mol_type = other RNA
tctcttatttggataggccag

SEQ ID NO: 83 - RNA, 21 nt, synthetic construct
  source 1..21: mol_type = other RNA
ggacagctctcatttcttgac

SEQ ID NO: 84 - RNA, 21 nt, synthetic construct
  source 1..21: mol_type = other RNA
caagaaatgagagctgtcctc

SEQ ID NO: 85 - RNA, 21 nt, synthetic construct
  source 1..21: mol_type = other RNA
gctctttgttccagtgttatc

SEQ ID NO: 86 - RNA, 21 nt, synthetic construct
  source 1..21: mol_type = other RNA
taacactggaacaaagagctc

SEQ ID NO: 87 - RNA, 21 nt, synthetic construct
  source 1..21: mol_type = other RNA
gccatgcactcatttccagcc

SEQ ID NO: 88 - RNA, 21 nt, synthetic construct
  source 1..21: mol_type = other RNA
ctggaaatgagtgcatggcat

SEQ ID NO: 89 - RNA, 21 nt, synthetic construct
  source 1..21: mol_type = other RNA
gcatgccagaagaatccaggg

SEQ ID NO: 90 - RNA, 21 nt, synthetic construct
  source 1..21: mol_type = other RNA
ctggattcttctggcatgcca

SEQ ID NO: 91 - RNA, 21 nt, synthetic construct
  source 1..21: mol_type = other RNA
gagcagctcattttagaagga

SEQ ID NO: 92 - RNA, 21 nt, synthetic construct
  source 1..21: mol_type = other RNA
cttctaaaatgagctgctcca

SEQ ID NO: 93 - RNA, 21 nt, synthetic construct
  source 1..21: mol_type = other RNA
gacctttattcctgactcttc

SEQ ID NO: 94 - RNA, 21 nt, synthetic construct
  source 1..21: mol_type = other RNA
agagtcaggaataaaggtcca

SEQ ID NO: 95 - RNA, 21 nt, synthetic construct
  source 1..21: mol_type = other RNA
gcacaggattgcactccacgt

SEQ ID NO: 96 - RNA, 21 nt, synthetic construct
  source 1..21: mol_type = other RNA
gtggagtgcaatcctgtgctg

SEQ ID NO: 97 - RNA, 21 nt, synthetic construct
  source 1..21: mol_type = other RNA
ggtgatcagatatcagatgaa

SEQ ID NO: 98 - RNA, 21 nt, synthetic construct
  source 1..21: mol_type = other RNA
catctgatatctgatcaccat

SEQ ID NO: 99 - RNA, 21 nt, synthetic construct
  source 1..21: mol_type = other RNA
gggtttaagtcttataggata

SEQ ID NO: 100 - RNA, 21 nt, synthetic construct
  source 1..21: mol_type = other RNA
tcctataagacttaaacccag

SEQ ID NO: 101 - RNA, 21 nt, synthetic construct
  source 1..21: mol_type = other RNA
ggaactcctgaataagcgagg

SEQ ID NO: 102 - RNA, 21 nt, synthetic construct
  source 1..21: mol_type = other RNA
tcgcttattcaggagttcctt

SEQ ID NO: 103 - RNA, 21 nt, synthetic construct
  source 1..21: mol_type = other RNA
ggactgaagaccttagatctc

SEQ ID NO: 104 - RNA, 21 nt, synthetic construct
  source 1..21: mol_type = other RNA
gatctaaggtcttcagtccaa

SEQ ID NO: 105 - RNA, 21 nt, synthetic construct
  source 1..21: mol_type = other RNA
ggtattaaacgaagtccatga

SEQ ID NO: 106 - RNA, 21 nt, synthetic construct
  source 1..21: mol_type = other RNA
atggacttcgtttaataccag

SEQ ID NO: 107 - RNA, 21 nt, synthetic construct
  source 1..21: mol_type = other RNA
ggcatagatgtgaaagactgg

SEQ ID NO: 108 - RNA, 21 nt, synthetic construct
  source 1..21: mol_type = other RNA
agtctttcacatctatgccaa

SEQ ID NO: 109 - RNA, 21 nt, synthetic construct
  source 1..21: mol_type = other RNA
ggatctgaagtcttagacaat

SEQ ID NO: 110 - RNA, 21 nt, synthetic construct
  source 1..21: mol_type = other RNA
tgtctaagacttcagatccta

SEQ ID NO: 111 - RNA, 21 nt, synthetic construct
  source 1..21: mol_type = other RNA
gttgtggaagtgtgggataag

SEQ ID NO: 112 - RNA, 21 nt, synthetic construct
  source 1..21: mol_type = other RNA
tatcccacacttccacaacag

SEQ ID NO: 113 - RNA, 21 nt, synthetic construct
  source 1..21: mol_type = other RNA
gatctgaagtcttagacaatc

SEQ ID NO: 114 - RNA, 21 nt, synthetic construct
  source 1..21: mol_type = other RNA
ttgtctaagacttcagatcct

SEQ ID NO: 115 - RNA, 21 nt, synthetic construct
  source 1..21: mol_type = other RNA
gagagagcgtgtgatcagaat

SEQ ID NO: 116 - RNA, 21 nt, synthetic construct
  source 1..21: mol_type = other RNA
tctgatcacacgctctctcta

SEQ ID NO: 117 - RNA, 21 nt, synthetic construct
  source 1..21: mol_type = other RNA
gtgaatgccaccgaggaatct

SEQ ID NO: 118 - RNA, 21 nt, synthetic construct
  source 1..21: mol_type = other RNA
attcctcggtggcattcacaa

SEQ ID NO: 119 - RNA, 21 nt, synthetic construct
  source 1..21: mol_type = other RNA
ggttgtccaaaacaccctaag

SEQ ID NO: 120 - RNA, 21 nt, synthetic construct
  source 1..21: mol_type = other RNA
tagggtgttttggacaacctt

SEQ ID NO: 121 - RNA, 21 nt, synthetic construct
  source 1..21: mol_type = other RNA
gctagagagagcgtgtgatca

SEQ ID NO: 122 - RNA, 21 nt, synthetic construct
  source 1..21: mol_type = other RNA
atcacacgctctctctagcat

SEQ ID NO: 123 - RNA, 21 nt, synthetic construct
  source 1..21: mol_type = other RNA
ggcgcttcgagctattttaca

SEQ ID NO: 124 - RNA, 21 nt, synthetic construct
  source 1..21: mol_type = other RNA
taaaatagctcgaagcgcctc

SEQ ID NO: 125 - RNA, 21 nt, synthetic construct
  source 1..21: mol_type = other RNA
gtgttcaccaattgattctta

SEQ ID NO: 126 - RNA, 21 nt, synthetic construct
  source 1..21: mol_type = other RNA
agaatcaattggtgaacacca

SEQ ID NO: 127 - RNA, 21 nt, synthetic construct
  source 1..21: mol_type = other RNA
ggtgttataatattgtggtgg

SEQ ID NO: 128 - RNA, 21 nt, synthetic construct
  source 1..21: mol_type = other RNA
accacaatattataacacctg

SEQ ID NO: 129 - RNA, 21 nt, synthetic construct
  source 1..21: mol_type = other RNA
gaaattatcatccgactatat

SEQ ID NO: 130 - RNA, 21 nt, synthetic construct
  source 1..21: mol_type = other RNA
atagtcggatgataatttcag

SEQ ID NO: 131 - RNA, 21 nt, synthetic construct
  source 1..21: mol_type = other RNA
gtttgagtgaatccacaaatt

SEQ ID NO: 132 - RNA, 21 nt, synthetic construct
  source 1..21: mol_type = other RNA
tttgtggattcactcaaacac

SEQ ID NO: 133 - RNA, 21 nt, synthetic construct
  source 1..21: mol_type = other RNA
gtggaccacattgattctctc

SEQ ID NO: 134 - RNA, 21 nt, synthetic construct
  source 1..21: mol_type = other RNA
gagaatcaatgtggtccacaa

SEQ ID NO: 135 - RNA, 21 nt, synthetic construct
  source 1..21: mol_type = other RNA
gaggacagctctcatttcttg

SEQ ID NO: 136 - RNA, 21 nt, synthetic construct
  source 1..21: mol_type = other RNA
agaaatgagagctgtcctctg

SEQ ID NO: 137 - RNA, 21 nt, synthetic construct
  source 1..21: mol_type = other RNA
gggaactcttatttagccata

SEQ ID NO: 138 - RNA, 21 nt, synthetic construct
  source 1..21: mol_type = other RNA
tggctaaataagagttccctt

SEQ ID NO: 139 - RNA, 21 nt, synthetic construct
  source 1..21: mol_type = other RNA
gggttggtcacttctgtgcaa

SEQ ID NO: 140 - RNA, 21 nt, synthetic construct
  source 1..21: mol_type = other RNA
gcacagaagtgaccaacccac

SEQ ID NO: 141 - RNA, 21 nt, synthetic construct
  source 1..21: mol_type = other RNA
gacgcagcgagcattgtacct

SEQ ID NO: 142 - RNA, 21 nt, synthetic construct
  source 1..21: mol_type = other RNA
gtacaatgctcgctgcgtcat

SEQ ID NO: 143 - RNA, 21 nt, synthetic construct
  source 1..21: mol_type = other RNA
gtactctcctggtcatcaata

SEQ ID NO: 144 - RNA, 21 nt, synthetic construct
  source 1..21: mol_type = other RNA
ttgatgaccaggagagtacca

SEQ ID NO: 145 - RNA, 21 nt, synthetic construct
  source 1..21: mol_type = other RNA
gctcctttgaagatactaaat

SEQ ID NO: 146 - RNA, 21 nt, synthetic construct
  source 1..21: mol_type = other RNA
ttagtatcttcaaaggagcag

SEQ ID NO: 147 - RNA, 21 nt, synthetic construct
  source 1..21: mol_type = other RNA
gcactcacgagctttccacaa

SEQ ID NO: 148 - RNA, 21 nt, synthetic construct
  source 1..21: mol_type = other RNA
gtggaaagctcgtgagtgcat

SEQ ID NO: 149 - RNA, 21 nt, synthetic construct
  source 1..21: mol_type = other RNA
gaacaagccaactgttttctt

SEQ ID NO: 150 - RNA, 21 nt, synthetic construct
  source 1..21: mol_type = other RNA
gaaaacagttggcttgttctt

SEQ ID NO: 151 - RNA, 21 nt, synthetic construct
  source 1..21: mol_type = other RNA
gaaatactgggagtggtaaaa

SEQ ID NO: 152 - RNA, 21 nt, synthetic construct
  source 1..21: mol_type = other RNA
ttaccactcccagtatttccc

SEQ ID NO: 153 - RNA, 21 nt, synthetic construct
  source 1..21: mol_type = other RNA
catctacactagcaagaatgg

SEQ ID NO: 154 - RNA, 21 nt, synthetic construct
  source 1..21: mol_type = other RNA
attcttgctagtgtagatgct

SEQ ID NO: 155 - RNA, 21 nt, synthetic construct
  source 1..21: mol_type = other RNA
gaactcctgaataagcgaggg

SEQ ID NO: 156 - RNA, 21 nt, synthetic construct
  source 1..21: mol_type = other RNA
ctcgcttattcaggagttcct

SEQ ID NO: 157 - RNA, 21 nt, synthetic construct
  source 1..21: mol_type = other RNA
ggaccacattgattctctcat

SEQ ID NO: 158 - RNA, 21 nt, synthetic construct
  source 1..21: mol_type = other RNA
gagagaatcaatgtggtccac

SEQ ID NO: 159 - RNA, 21 nt, synthetic construct
  source 1..21: mol_type = other RNA
ggacgaggaaactctgaagaa

SEQ ID NO: 160 - RNA, 21 nt, synthetic construct
  source 1..21: mol_type = other RNA
cttcagagtttcctcgtcctc

SEQ ID NO: 161 - RNA, 21 nt, synthetic construct
  source 1..21: mol_type = other RNA
gcgagcattgtaccttgctgt

SEQ ID NO: 162 - RNA, 21 nt, synthetic construct
  source 1..21: mol_type = other RNA
agcaaggtacaatgctcgctg

SEQ ID NO: 163 - RNA, 21 nt, synthetic construct
  source 1..21: mol_type = other RNA
gtgcttagccttggtgcatct

SEQ ID NO: 164 - RNA, 21 nt, synthetic construct
  source 1..21: mol_type = other RNA
atgcaccaaggctaagcacaa

SEQ ID NO: 165 - RNA, 21 nt, synthetic construct
  source 1..21: mol_type = other RNA
ggtcagagtcatgatgacagc

SEQ ID NO: 166 - RNA, 21 nt, synthetic construct
  source 1..21: mol_type = other RNA
tgtcatcatgactctgaccga

SEQ ID NO: 167 - RNA, 21 nt, synthetic construct
  source 1..21: mol_type = other RNA
gtcatataaccagctgtcttt

SEQ ID NO: 168 - RNA, 21 nt, synthetic construct
  source 1..21: mol_type = other RNA
agacagctggttatatgacag

SEQ ID NO: 169 - RNA, 21 nt, synthetic construct
  source 1..21: mol_type = other RNA
ggaggccatattttactcctg

SEQ ID NO: 170 - RNA, 21 nt, synthetic construct
  source 1..21: mol_type = other RNA
ggagtaaaatatggcctcctc

SEQ ID NO: 171 - RNA, 21 nt, synthetic construct
  source 1..21: mol_type = other RNA
gagtctcagaggagcaactga

SEQ ID NO: 172 - RNA, 21 nt, synthetic construct
  source 1..21: mol_type = other RNA
agttgctcctctgagactctc

SEQ ID NO: 173 - RNA, 21 nt, synthetic construct
  source 1..21: mol_type = other RNA
ggaagcctgttacaaagcatt

SEQ ID NO: 174 - RNA, 21 nt, synthetic construct
  source 1..21: mol_type = other RNA
tgctttgtaacaggcttccag

SEQ ID NO: 175 - RNA, 21 nt, synthetic construct
  source 1..21: mol_type = other RNA
gatgccctaagccagaaatgc

SEQ ID NO: 176 - RNA, 21 nt, synthetic construct
  source 1..21: mol_type = other RNA
atttctggcttagggcatcaa

SEQ ID NO: 177 - RNA, 21 nt, synthetic construct
  source 1..21: mol_type = other RNA
gattggctgtcttgaaaatct

SEQ ID NO: 178 - RNA, 21 nt, synthetic construct
  source 1..21: mol_type = other RNA
attttcaagacagccaatctc

SEQ ID NO: 179 - RNA, 21 nt, synthetic construct
  source 1..21: mol_type = other RNA
gactggcctatccaaataaga

SEQ ID NO: 180 - RNA, 21 nt, synthetic construct
  source 1..21: mol_type = other RNA
ttatttggataggccagtctt

SEQ ID NO: 181 - RNA, 21 nt, synthetic construct
  source 1..21: mol_type = other RNA
gacataccctagaaaagatga

SEQ ID NO: 182 - RNA, 21 nt, synthetic construct
  source 1..21: mol_type = other RNA
atcttttctagggtatgtctc

SEQ ID NO: 183 - RNA, 21 nt, synthetic construct
  source 1..21: mol_type = other RNA
gatgtctctcgaaatgacatt

SEQ ID NO: 184 - RNA, 21 nt, synthetic construct
  source 1..21: mol_type = other RNA
tgtcatttcgagagacatcaa

SEQ ID NO: 185 - RNA, 21 nt, synthetic construct
  source 1..21: mol_type = other RNA
ggtcatttctgctttaggaaa

SEQ ID NO: 186 - RNA, 21 nt, synthetic construct
  source 1..21: mol_type = other RNA
tcctaaagcagaaatgacctc

SEQ ID NO: 187 - RNA, 21 nt, synthetic construct
  source 1..21: mol_type = other RNA
gagcgtgtgatcagaataaca

SEQ ID NO: 188 - RNA, 21 nt, synthetic construct
  source 1..21: mol_type = other RNA
ttattctgatcacacgctctc

SEQ ID NO: 189 - RNA, 21 nt, synthetic construct
  source 1..21: mol_type = other RNA
gatctcgtgaacaagatgtac

SEQ ID NO: 190 - RNA, 21 nt, synthetic construct
  source 1..21: mol_type = other RNA
acatcttgttcacgagatcca

SEQ ID NO: 191 - RNA, 21 nt, synthetic construct
  source 1..21: mol_type = other RNA
ggacttgcacagtaataaatt

SEQ ID NO: 192 - RNA, 21 nt, synthetic construct
  source 1..21: mol_type = other RNA
tttattactgtgcaagtccaa

SEQ ID NO: 193 - RNA, 21 nt, synthetic construct
  source 1..21: mol_type = other RNA
ggtcgtgaggaattctatagt

SEQ ID NO: 194 - RNA, 21 nt, synthetic construct
  source 1..21: mol_type = other RNA
tatagaattcctcacgacctg

SEQ ID NO: 195 - RNA, 21 nt, synthetic construct
  source 1..21: mol_type = other RNA
ggccaaatacctatgtaataa

SEQ ID NO: 196 - RNA, 21 nt, synthetic construct
  source 1..21: mol_type = other RNA
attacataggtatttggccct

SEQ ID NO: 197 - RNA, 21 nt, synthetic construct
  source 1..21: mol_type = other RNA
ggtctttagatatgagcagca

SEQ ID NO: 198 - RNA, 21 nt, synthetic construct
  source 1..21: mol_type = other RNA
ctgctcatatctaaagaccgc

SEQ ID NO: 199 - RNA, 21 nt, synthetic construct
  source 1..21: mol_type = other RNA
gacgaggaaactctgaagaag

SEQ ID NO: 200 - RNA, 21 nt, synthetic construct
  source 1..21: mol_type = other RNA
tcttcagagtttcctcgtcct

SEQ ID NO: 201 - RNA, 19 nt, synthetic construct
  source 1..19: mol_type = other RNA
  modified_base 1: mod_base = um
  modified_base 2: mod_base = gm
  modified_base 3: mod_base = gm
  modified_base 4: mod_base = um
  modified_base 5: mod_base = OTHER; note = 2'-deoxy-2'-fluoro uridine
  modified_base 6: mod_base = OTHER; note = 2'-O-methyl adenosine
  modified_base 7: mod_base = OTHER; note = 2'-deoxy-2'-fluoro adenosine
  modified_base 8: mod_base = OTHER; note = 2'-deoxy-2'-fluoro cytidine
  modified_base 10: mod_base = um
  modified_base 9: mod_base = OTHER; note = 2'-deoxy-2'-fluoro adenosine
  modified_base 11: mod_base = gm
  modified_base 12: mod_base = um
  modified_base 13: mod_base = cm
  modified_base 16: mod_base = cm
  modified_base 14: mod_base = gm
  modified_base 15: mod_base = OTHER; note = 2'-O-methyl adenosine
  modified_base 17: mod_base = um
  modified_base 18: mod_base = OTHER; note = 2'-O-methyl adenosine
  modified_base 19: mod_base = OTHER; note = 2'-O-methyl adenosine
  modified_base 1..3: mod_base = OTHER; note = phosphorothioate (PS) linkage
  modified_base 18^19: mod_base = OTHER; note = phosphorothioate (PS) linkage
  misc_feature 19: note = The Am is attached through the phosphorothioate linkage to [C5Cholesterol]
tggttaacatgtcgactaa

SEQ ID NO: 202 - RNA, 21 nt, synthetic construct
  source 1..21: mol_type = other RNA
  modified_base 5: mod_base = um
  modified_base 6: mod_base = OTHER; note = 2'-deoxy-2'-fluoro cytidine
  modified_base 7: mod_base = gm
  modified_base 8: mod_base = OTHER; note = 2'-deoxy-2'-fluoro adenosine
  modified_base 10: mod_base = OTHER; note = 2'-O-methyl adenosine
  modified_base 9: mod_base = OTHER; note = 2'-deoxy-2'-fluoro cytidine
  modified_base 11: mod_base = um
  modified_base 12: mod_base = gm
  modified_base 16: mod_base = OTHER; note = 2'-deoxy-2'-fluoro adenosine
  modified_base 13: mod_base = gm
  modified_base 15: mod_base = OTHER; note = 2'-O-methyl adenosine
  modified_base 14: mod_base = OTHER; note = 2'-deoxy-2'-fluoro adenosine
  modified_base 1: mod_base = OTHER; note = vinyl phosphonate 2'-O-methyl uridine
  modified_base 2: mod_base = OTHER; note = 2'-deoxy-2'-fluoro uridine
  modified_base 3: mod_base = OTHER; note = 2'-O-methyl adenosine
  modified_base 4: mod_base = gm
  modified_base 17: mod_base = cm
  modified_base 18: mod_base = cm
  modified_base 19: mod_base = OTHER; note = 2'-O-methyl adenosine
  modified_base 20: mod_base = um
  modified_base 21: mod_base = um
  modified_base 1..3: mod_base = OTHER; note = phosphorothioate (PS) linkage
  modified_base 19..21: mod_base = OTHER; note = phosphorothioate (PS) linkage
ttagtcgacatggaaaccatt

SEQ ID NO: 203 - RNA, 19 nt, synthetic construct
  source 1..19: mol_type = other RNA
  modified_base 1: mod_base = OTHER; note = 2'-O-methyl adenosine
  modified_base 2: mod_base = gm
  modified_base 3: mod_base = OTHER; note = 2'-O-methyl adenosine
  modified_base 4: mod_base = gm
  modified_base 5: mod_base = OTHER; note = 2'-deoxy-2'-fluoro adenosine
  modified_base 6: mod_base = OTHER; note = 2'-O-methyl adenosine
  modified_base 7: mod_base = OTHER; note = 2'-deoxy-2'-fluoro adenosine
  modified_base 8: mod_base = OTHER; note = 2'-deoxy-2'-fluoro adenosine
  modified_base 9: mod_base = OTHER; note = 2'-deoxy-2'-fluoro guanosine
  modified_base 10: mod_base = OTHER; note = 2'-O-methyl adenosine
  modified_base 11: mod_base = gm
  modified_base 12: mod_base = OTHER; note = 2'-O-methyl adenosine
  modified_base 13: mod_base = OTHER; note = 2'-O-methyl adenosine
  modified_base 14: mod_base = OTHER; note = 2'-O-methyl adenosine
  modified_base 15: mod_base = gm
  modified_base 16: mod_base = um
  modified_base 17: mod_base = gm
  modified_base 18: mod_base = um
  modified_base 19: mod_base = um
  modified_base 1..3: mod_base = OTHER; note = phosphorothioate (PS) linkage
  modified_base 18^19: mod_base = OTHER; note = phosphorothioate (PS) linkage
  misc_feature 19: note = The Um is attached through the phosphorothioate linkage to [C5Cholesterol]
agagaaaagagaaagtgtt

SEQ ID NO: 204 - RNA, 21 nt, synthetic construct
  source 1..21: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = cm
  modified_base 4: mod_base = OTHER; note = 2'-O-methyl adenosine
  modified_base 5: mod_base = cm
  modified_base 6: mod_base = OTHER; note = 2'-deoxy-2'-fluoro uridine
  modified_base 7: mod_base = um
  modified_base 8: mod_base = OTHER; note = 2'-deoxy-2'-fluoro uridine
  modified_base 9: mod_base = OTHER; note = 2'-deoxy-2'-fluoro cytidine
  modified_base 10: mod_base = um
  modified_base 11: mod_base = cm
  modified_base 12: mod_base = um
  modified_base 13: mod_base = um
  modified_base 14: mod_base = OTHER; note = 2'-deoxy-2'-fluoro uridine
  modified_base 15: mod_base = um
  modified_base 16: mod_base = OTHER; note = 2'-deoxy-2'-fluoro cytidine
  modified_base 17: mod_base = um
  modified_base 18: mod_base = cm
  modified_base 19: mod_base = um
  modified_base 20: mod_base = um
  modified_base 21: mod_base = um
  modified_base 1..3: mod_base = OTHER; note = phosphorothioate (PS) linkage
  modified_base 19..21: mod_base = OTHER; note = phosphorothioate (PS) linkage
tacactttctcttttctcttt

SEQ ID NO: 205 - RNA, 19 nt, synthetic construct
  source 1..19: mol_type = other RNA
  modified_base 1: mod_base = OTHER; note = 2'-O-methyl adenosine
  modified_base 2: mod_base = gm
  modified_base 3: mod_base = OTHER; note = 2'-O-methyl adenosine
  modified_base 4: mod_base = gm
  modified_base 5: mod_base = OTHER; note = 2'-deoxy-2'-fluoro adenosine
  modified_base 6: mod_base = OTHER; note = 2'-O-methyl adenosine
  modified_base 7: mod_base = OTHER; note = 2'-deoxy-2'-fluoro adenosine
  modified_base 8: mod_base = OTHER; note = 2'-deoxy-2'-fluoro adenosine
  modified_base 9: mod_base = OTHER; note = 2'-deoxy-2'-fluoro guanosine
  modified_base 10: mod_base = OTHER; note = 2'-O-methyl adenosine
  modified_base 11: mod_base = gm
  modified_base 12: mod_base = OTHER; note = 2'-O-methyl adenosine
  modified_base 13: mod_base = OTHER; note = 2'-O-methyl adenosine
  modified_base 14: mod_base = OTHER; note = 2'-O-methyl adenosine
  modified_base 15: mod_base = gm
  modified_base 16: mod_base = um
  modified_base 17: mod_base = gm
  modified_base 18: mod_base = um
  modified_base 19: mod_base = um
  modified_base 1..3: mod_base = OTHER; note = phosphorothioate (PS) linkage
  modified_base 18^19: mod_base = OTHER; note = phosphorothioate (PS) linkage
  misc_feature 19: note = The Um is attached through the phosphorothioate linkage to [N3T12-010-01]
agagaaaagagaaagtgtt

SEQ ID NO: 206 - RNA, 21 nt, synthetic construct
  source 1..21: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = cm
  modified_base 4: mod_base = OTHER; note = 2'-O-methyl adenosine
  modified_base 5: mod_base = cm
  modified_base 6: mod_base = OTHER; note = 2'-deoxy-2'-fluoro uridine
  modified_base 7: mod_base = um
  modified_base 8: mod_base = OTHER; note = 2'-deoxy-2'-fluoro uridine
  modified_base 9: mod_base = OTHER; note = 2'-deoxy-2'-fluoro cytidine
  modified_base 10: mod_base = um
  modified_base 11: mod_base = cm
  modified_base 12: mod_base = um
  modified_base 13: mod_base = um
  modified_base 14: mod_base = OTHER; note = 2'-deoxy-2'-fluoro uridine
  modified_base 15: mod_base = um
  modified_base 16: mod_base = OTHER; note = 2'-deoxy-2'-fluoro cytidine
  modified_base 17: mod_base = um
  modified_base 18: mod_base = cm
  modified_base 19: mod_base = um
  modified_base 20: mod_base = um
  modified_base 21: mod_base = um
  modified_base 1..3: mod_base = OTHER; note = phosphorothioate (PS) linkage
  modified_base 19..21: mod_base = OTHER; note = phosphorothioate (PS) linkage
tacactttctcttttctcttt

SEQ ID NO: 207 - RNA, 19 nt, synthetic construct
  source 1..19: mol_type = other RNA
  modified_base 1: mod_base = OTHER; note = 2'-O-methyl adenosine
  modified_base 2: mod_base = gm
  modified_base 3: mod_base = OTHER; note = 2'-O-methyl adenosine
  modified_base 4: mod_base = gm
  modified_base 5: mod_base = OTHER; note = 2'-deoxy-2'-fluoro adenosine
  modified_base 6: mod_base = OTHER; note = 2'-O-methyl adenosine
  modified_base 7: mod_base = OTHER; note = 2'-deoxy-2'-fluoro adenosine
  modified_base 8: mod_base = OTHER; note = 2'-deoxy-2'-fluoro adenosine
  modified_base 9: mod_base = OTHER; note = 2'-deoxy-2'-fluoro guanosine
  modified_base 10: mod_base = OTHER; note = 2'-O-methyl adenosine
  modified_base 11: mod_base = gm
  modified_base 12: mod_base = OTHER; note = 2'-O-methyl adenosine
  modified_base 13: mod_base = OTHER; note = 2'-O-methyl adenosine
  modified_base 14: mod_base = OTHER; note = 2'-O-methyl adenosine
  modified_base 15: mod_base = gm
  modified_base 16: mod_base = um
  modified_base 17: mod_base = gm
  modified_base 18: mod_base = um
  modified_base 19: mod_base = um
  modified_base 1..3: mod_base = OTHER; note = phosphorothioate (PS) linkage
  modified_base 18^19: mod_base = OTHER; note = phosphorothioate (PS) linkage
  misc_feature 19: note = The Um is attached through the phosphorothioate linkage to [N3T12-012-01]
agagaaaagagaaagtgtt

SEQ ID NO: 208 - RNA, 21 nt, synthetic construct
  source 1..21: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = cm
  modified_base 4: mod_base = OTHER; note = 2'-O-methyl adenosine
  modified_base 5: mod_base = cm
  modified_base 6: mod_base = OTHER; note = 2'-deoxy-2'-fluoro uridine
  modified_base 7: mod_base = um
  modified_base 8: mod_base = OTHER; note = 2'-deoxy-2'-fluoro uridine
  modified_base 9: mod_base = OTHER; note = 2'-deoxy-2'-fluoro cytidine
  modified_base 10: mod_base = um
  modified_base 11: mod_base = cm
  modified_base 12: mod_base = um
  modified_base 13: mod_base = um
  modified_base 14: mod_base = OTHER; note = 2'-deoxy-2'-fluoro uridine
  modified_base 15: mod_base = um
  modified_base 16: mod_base = OTHER; note = 2'-deoxy-2'-fluoro cytidine
  modified_base 17: mod_base = um
  modified_base 18: mod_base = cm
  modified_base 19: mod_base = um
  modified_base 20: mod_base = um
  modified_base 21: mod_base = um
  modified_base 1..3: mod_base = OTHER; note = phosphorothioate (PS) linkage
  modified_base 19..21: mod_base = OTHER; note = phosphorothioate (PS) linkage
tacactttctcttttctcttt

SEQ ID NO: 209 - RNA, 19 nt, synthetic construct
  source 1..19: mol_type = other RNA
  modified_base 1: mod_base = OTHER; note = 2'-O-methyl adenosine
  modified_base 2: mod_base = gm
  modified_base 3: mod_base = OTHER; note = 2'-O-methyl adenosine
  modified_base 4: mod_base = gm
  modified_base 5: mod_base = OTHER; note = 2'-deoxy-2'-fluoro adenosine
  modified_base 6: mod_base = OTHER; note = 2'-O-methyl adenosine
  modified_base 7: mod_base = OTHER; note = 2'-deoxy-2'-fluoro adenosine
  modified_base 8: mod_base = OTHER; note = 2'-deoxy-2'-fluoro adenosine
  modified_base 9: mod_base = OTHER; note = 2'-deoxy-2'-fluoro guanosine
  modified_base 10: mod_base = OTHER; note = 2'-O-methyl adenosine
  modified_base 11: mod_base = gm
  modified_base 12: mod_base = OTHER; note = 2'-O-methyl adenosine
  modified_base 13: mod_base = OTHER; note = 2'-O-methyl adenosine
  modified_base 14: mod_base = OTHER; note = 2'-O-methyl adenosine
  modified_base 15: mod_base = gm
  modified_base 16: mod_base = um
  modified_base 17: mod_base = gm
  modified_base 18: mod_base = um
  modified_base 19: mod_base = um
  modified_base 1..3: mod_base = OTHER; note = phosphorothioate (PS) linkage
  modified_base 18^19: mod_base = OTHER; note = phosphorothioate (PS) linkage
  misc_feature 19: note = The Um is attached through the phosphorothioate linkage to [N3T12-023-01]
agagaaaagagaaagtgtt

SEQ ID NO: 210 - RNA, 21 nt, synthetic construct
  source 1..21: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = cm
  modified_base 4: mod_base = OTHER; note = 2'-O-methyl adenosine
  modified_base 5: mod_base = cm
  modified_base 6: mod_base = OTHER; note = 2'-deoxy-2'-fluoro uridine
  modified_base 7: mod_base = um
  modified_base 8: mod_base = OTHER; note = 2'-deoxy-2'-fluoro uridine
  modified_base 9: mod_base = OTHER; note = 2'-deoxy-2'-fluoro cytidine
  modified_base 10: mod_base = um
  modified_base 11: mod_base = cm
  modified_base 12: mod_base = um
  modified_base 13: mod_base = um
  modified_base 14: mod_base = OTHER; note = 2'-deoxy-2'-fluoro uridine
  modified_base 15: mod_base = um
  modified_base 16: mod_base = OTHER; note = 2'-deoxy-2'-fluoro cytidine
  modified_base 17: mod_base = um
  modified_base 18: mod_base = cm
  modified_base 19: mod_base = um
  modified_base 20: mod_base = um
  modified_base 21: mod_base = um
  modified_base 1..3: mod_base = OTHER; note = phosphorothioate (PS) linkage
  modified_base 19..21: mod_base = OTHER; note = phosphorothioate (PS) linkage
tacactttctcttttctcttt

SEQ ID NO: 211 - RNA, 19 nt, synthetic construct
  source 1..19: mol_type = other RNA
  modified_base 1: mod_base = OTHER; note = 2'-O-methyl adenosine
  modified_base 2: mod_base = gm
  modified_base 3: mod_base = OTHER; note = 2'-O-methyl adenosine
  modified_base 4: mod_base = gm
  modified_base 5: mod_base = OTHER; note = 2'-deoxy-2'-fluoro adenosine
  modified_base 6: mod_base = OTHER; note = 2'-O-methyl adenosine
  modified_base 7: mod_base = OTHER; note = 2'-deoxy-2'-fluoro adenosine
  modified_base 8: mod_base = OTHER; note = 2'-deoxy-2'-fluoro adenosine
  modified_base 9: mod_base = OTHER; note = 2'-deoxy-2'-fluoro guanosine
  modified_base 10: mod_base = OTHER; note = 2'-O-methyl adenosine
  modified_base 11: mod_base = gm
  modified_base 12: mod_base = OTHER; note = 2'-O-methyl adenosine
  modified_base 13: mod_base = OTHER; note = 2'-O-methyl adenosine
  modified_base 14: mod_base = OTHER; note = 2'-O-methyl adenosine
  modified_base 15: mod_base = gm
  modified_base 16: mod_base = um
  modified_base 17: mod_base = gm
  modified_base 18: mod_base = um
  modified_base 19: mod_base = um
  modified_base 1^2: mod_base = OTHER; note = phosphorothioate (PS) linkage
  misc_feature 1: note = The Am is attached through the phosphorothioate linkage to [N3T12-019-01]
  modified_base 17..19: mod_base = OTHER; note = phosphorothioate (PS) linkage
agagaaaagagaaagtgtt

SEQ ID NO: 212 - RNA, 21 nt, synthetic construct
  source 1..21: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = cm
  modified_base 4: mod_base = OTHER; note = 2'-O-methyl adenosine
  modified_base 5: mod_base = cm
  modified_base 6: mod_base = OTHER; note = 2'-deoxy-2'-fluoro uridine
  modified_base 7: mod_base = um
  modified_base 8: mod_base = OTHER; note = 2'-deoxy-2'-fluoro uridine
  modified_base 9: mod_base = OTHER; note = 2'-deoxy-2'-fluoro cytidine
  modified_base 10: mod_base = um
  modified_base 11: mod_base = cm
  modified_base 12: mod_base = um
  modified_base 13: mod_base = um
  modified_base 14: mod_base = OTHER; note = 2'-deoxy-2'-fluoro uridine
  modified_base 15: mod_base = um
  modified_base 16: mod_base = OTHER; note = 2'-deoxy-2'-fluoro cytidine
  modified_base 17: mod_base = um
  modified_base 18: mod_base = cm
  modified_base 19: mod_base = um
  modified_base 20: mod_base = um
  modified_base 21: mod_base = um
  modified_base 1..3: mod_base = OTHER; note = phosphorothioate (PS) linkage
  modified_base 19..21: mod_base = OTHER; note = phosphorothioate (PS) linkage
tacactttctcttttctcttt

SEQ ID NO: 213 - RNA, 19 nt, synthetic construct
  source 1..19: mol_type = other RNA
  modified_base 1: mod_base = OTHER; note = 2'-O-methyl adenosine
  modified_base 2: mod_base = gm
  modified_base 3: mod_base = OTHER; note = 2'-O-methyl adenosine
  modified_base 4: mod_base = gm
  modified_base 5: mod_base = OTHER; note = 2'-deoxy-2'-fluoro adenosine
  modified_base 6: mod_base = OTHER; note = 2'-O-methyl adenosine
  modified_base 7: mod_base = OTHER; note = 2'-deoxy-2'-fluoro adenosine
  modified_base 8: mod_base = OTHER; note = 2'-deoxy-2'-fluoro adenosine
  modified_base 9: mod_base = OTHER; note = 2'-deoxy-2'-fluoro guanosine
  modified_base 10: mod_base = OTHER; note = 2'-O-methyl adenosine
  modified_base 11: mod_base = gm
  modified_base 12: mod_base = OTHER; note = 2'-O-methyl adenosine
  modified_base 13: mod_base = OTHER; note = 2'-O-methyl adenosine
  modified_base 14: mod_base = OTHER; note = 2'-O-methyl adenosine
  modified_base 15: mod_base = gm
  modified_base 16: mod_base = um
  modified_base 17: mod_base = gm
  modified_base 18: mod_base = um
  modified_base 19: mod_base = um
  modified_base 1^2: mod_base = OTHER; note = phosphorothioate (PS) linkage
  modified_base 18^19: mod_base = OTHER; note = phosphorothioate (PS) linkage
  misc_feature 19: note = The Um is attached through the phosphorothioate linkage to [N3T12-023-01]
  misc_feature 1: note = The Am is attached through the phosphorothioate linkage to [N3T12-019-01]
agagaaaagagaaagtgtt

SEQ ID NO: 214 - RNA, 21 nt, synthetic construct
  source 1..21: mol_type = other RNA
  modified_base 1: mod_base = OTHER; note = 2'-deoxy-2'-fluoro guanosine
  modified_base 2: mod_base = OTHER; note = 2'-deoxy-2'-fluoro adenosine
  modified_base 3: mod_base = cm
  modified_base 4: mod_base = OTHER; note = 2'-O-methyl adenosine
  modified_base 5: mod_base = cm
  modified_base 6: mod_base = OTHER; note = 2'-deoxy-2'-fluoro uridine
  modified_base 7: mod_base = um
  modified_base 8: mod_base = OTHER; note = 2'-deoxy-2'-fluoro uridine
  modified_base 9: mod_base = OTHER; note = 2'-deoxy-2'-fluoro cytidine
  modified_base 10: mod_base = um
  modified_base 11: mod_base = cm
  modified_base 12: mod_base = um
  modified_base 13: mod_base = um
  modified_base 14: mod_base = OTHER; note = 2'-deoxy-2'-fluoro uridine
  modified_base 15: mod_base = um
  modified_base 16: mod_base = OTHER; note = 2'-deoxy-2'-fluoro cytidine
  modified_base 17: mod_base = um
  modified_base 18: mod_base = cm
  modified_base 19: mod_base = um
  modified_base 20: mod_base = um
  modified_base 21: mod_base = um
  modified_base 1..3: mod_base = OTHER; note = phosphorothioate (PS) linkage
  modified_base 19..21: mod_base = OTHER; note = phosphorothioate (PS) linkage
tacactttctcttttctcttt

SEQ ID NO: 215 - RNA, 13 nt, synthetic construct
  source 1..13: mol_type = other RNA
  modified_base 1: mod_base = OTHER; note = 2'-deoxy-2'-fluoro adenosine
  modified_base 2: mod_base = OTHER; note = 2'-deoxy-2'-fluoro adenosine
  modified_base 3: mod_base = OTHER; note = 2'-deoxy-2'-fluoro guanosine
  modified_base 4: mod_base = OTHER; note = 2'-O-methyl adenosine
  modified_base 5: mod_base = gm
  modified_base 6: mod_base = OTHER; note = 2'-O-methyl adenosine
  modified_base 8: mod_base = OTHER; note = 2'-O-methyl adenosine
  modified_base 7: mod_base = OTHER; note = 2'-O-methyl adenosine
  modified_base 9: mod_base = gm
  modified_base 10: mod_base = um
  modified_base 12: mod_base = um
  modified_base 11..13: mod_base = OTHER; note = phosphorothioate (PS) linkage
  misc_feature 1: note = The Af is attached to AmsGmsAmGmAf[N3T12-022-01]
  modified_base 11: mod_base = gm
  modified_base 13: mod_base = um
aagagaaagtgtt

SEQ ID NO: 216 - RNA, 21 nt, synthetic construct
  source 1..21: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = cm
  modified_base 4: mod_base = OTHER; note = 2'-O-methyl adenosine
  modified_base 5: mod_base = cm
  modified_base 6: mod_base = OTHER; note = 2'-deoxy-2'-fluoro uridine
  modified_base 7: mod_base = um
  modified_base 8: mod_base = OTHER; note = 2'-deoxy-2'-fluoro uridine
  modified_base 9: mod_base = OTHER; note = 2'-deoxy-2'-fluoro cytidine
  modified_base 10: mod_base = um
  modified_base 11: mod_base = cm
  modified_base 12: mod_base = um
  modified_base 13: mod_base = um
  modified_base 14: mod_base = OTHER; note = 2'-deoxy-2'-fluoro uridine
  modified_base 15: mod_base = um
  modified_base 16: mod_base = OTHER; note = 2'-deoxy-2'-fluoro cytidine
  modified_base 17: mod_base = um
  modified_base 18: mod_base = cm
  modified_base 19: mod_base = um
  modified_base 20: mod_base = um
  modified_base 21: mod_base = um
  modified_base 1..3: mod_base = OTHER; note = phosphorothioate (PS) linkage
  modified_base 19..21: mod_base = OTHER; note = phosphorothioate (PS) linkage
tacactttctcttttctcttt

SEQ ID NO: 217 - RNA, 19 nt, synthetic construct
  source 1..19: mol_type = other RNA
  modified_base 1: mod_base = OTHER; note = 2'-O-methyl adenosine
  modified_base 2: mod_base = gm
  modified_base 3: mod_base = OTHER; note = 2'-O-methyl adenosine
  modified_base 4: mod_base = gm
  modified_base 5: mod_base = OTHER; note = 2'-deoxy-2'-fluoro adenosine
  modified_base 6: mod_base = OTHER; note = 2'-O-methyl adenosine
  modified_base 7: mod_base = OTHER; note = 2'-deoxy-2'-fluoro adenosine
  modified_base 8: mod_base = OTHER; note = 2'-deoxy-2'-fluoro adenosine
  modified_base 9: mod_base = OTHER; note = 2'-deoxy-2'-fluoro guanosine
  modified_base 10: mod_base = OTHER; note = 2'-O-methyl adenosine
  modified_base 11: mod_base = gm
  modified_base 12: mod_base = OTHER; note = 2'-O-methyl adenosine
  modified_base 13: mod_base = OTHER; note = 2'-O-methyl adenosine
  modified_base 14: mod_base = OTHER; note = 2'-O-methyl adenosine
  modified_base 15: mod_base = gm
  modified_base 16: mod_base = um
  modified_base 17: mod_base = gm
  modified_base 18: mod_base = um
  modified_base 19: mod_base = um
  modified_base 1^2: mod_base = OTHER; note = phosphorothioate (PS) linkage
  modified_base 17..19: mod_base = OTHER; note = phosphorothioate (PS) linkage
  misc_feature 1: note = The Am is attached through the phosphorothioate linkage to [N3T12-018-01]
agagaaaagagaaagtgtt

SEQ ID NO: 218 - RNA, 21 nt, synthetic construct
  source 1..21: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = cm
  modified_base 4: mod_base = OTHER; note = 2'-O-methyl adenosine
  modified_base 5: mod_base = cm
  modified_base 6: mod_base = OTHER; note = 2'-deoxy-2'-fluoro uridine
  modified_base 7: mod_base = um
  modified_base 8: mod_base = OTHER; note = 2'-deoxy-2'-fluoro uridine
  modified_base 9: mod_base = OTHER; note = 2'-deoxy-2'-fluoro cytidine
  modified_base 10: mod_base = um
  modified_base 11: mod_base = cm
  modified_base 12: mod_base = um
  modified_base 13: mod_base = um
  modified_base 14: mod_base = OTHER; note = 2'-deoxy-2'-fluoro uridine
  modified_base 15: mod_base = um
  modified_base 16: mod_base = OTHER; note = 2'-deoxy-2'-fluoro cytidine
  modified_base 17: mod_base = um
  modified_base 18: mod_base = cm
  modified_base 19: mod_base = um
  modified_base 20: mod_base = um
  modified_base 21: mod_base = um
  modified_base 1..3: mod_base = OTHER; note = phosphorothioate (PS) linkage
  modified_base 19..21: mod_base = OTHER; note = phosphorothioate (PS) linkage
tacactttctcttttctcttt

SEQ ID NO: 219 - RNA, 13 nt, synthetic construct
  source 1..13: mol_type = other RNA
  modified_base 1: mod_base = OTHER; note = 2'-deoxy-2'-fluoro adenosine
  modified_base 2: mod_base = OTHER; note = 2'-deoxy-2'-fluoro adenosine
  modified_base 3: mod_base = OTHER; note = 2'-deoxy-2'-fluoro guanosine
  modified_base 4: mod_base = OTHER; note = 2'-O-methyl adenosine
  modified_base 5: mod_base = gm
  modified_base 6: mod_base = OTHER; note = 2'-O-methyl adenosine
  modified_base 7: mod_base = OTHER; note = 2'-O-methyl adenosine
  modified_base 8: mod_base = OTHER; note = 2'-O-methyl adenosine
  modified_base 9: mod_base = gm
  modified_base 10: mod_base = um
  modified_base 11: mod_base = gm
  modified_base 12: mod_base = um
  modified_base 13: mod_base = um
  modified_base 11..13: mod_base = OTHER; note = phosphorothioate (PS) linkage
  misc_feature 1: note = The Af is attached to AmsGmsAmGmAf[Ahd]
aagagaaagtgtt

SEQ ID NO: 220 - RNA, 21 nt, synthetic construct
  source 1..21: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = cm
  modified_base 4: mod_base = OTHER; note = 2'-O-methyl adenosine
  modified_base 5: mod_base = cm
  modified_base 6: mod_base = OTHER; note = 2'-deoxy-2'-fluoro uridine
  modified_base 7: mod_base = um
  modified_base 8: mod_base = OTHER; note = 2'-deoxy-2'-fluoro uridine
  modified_base 9: mod_base = OTHER; note = 2'-deoxy-2'-fluoro cytidine
  modified_base 10: mod_base = um
  modified_base 11: mod_base = cm
  modified_base 12: mod_base = um
  modified_base 13: mod_base = um
  modified_base 14: mod_base = OTHER; note = 2'-deoxy-2'-fluoro uridine
  modified_base 15: mod_base = um
  modified_base 16: mod_base = OTHER; note = 2'-deoxy-2'-fluoro cytidine
  modified_base 17: mod_base = um
  modified_base 18: mod_base = cm
  modified_base 19: mod_base = um
  modified_base 20: mod_base = um
  modified_base 21: mod_base = um
  modified_base 1..3: mod_base = OTHER; note = phosphorothioate (PS) linkage
  modified_base 19..21: mod_base = OTHER; note = phosphorothioate (PS) linkage
tacactttctcttttctcttt

SEQ ID NO: 221 - RNA, 19 nt, synthetic construct
  source 1..19: mol_type = other RNA
ggcaatgtcaggtgttata

SEQ ID NO: 222 - RNA, 21 nt, synthetic construct
  source 1..21: mol_type = other RNA
gtggcaatgtcaggtgttata

SEQ ID NO: 223 - RNA, 23 nt, synthetic construct
  source 1..23: mol_type = other RNA
tataacacctgacattgccactc

SEQ ID NO: 224 - RNA, 19 nt, synthetic construct
  source 1..19: mol_type = other RNA
ggacttgcacagtaataaa

SEQ ID NO: 225 - RNA, 21 nt, synthetic construct
  source 1..21: mol_type = other RNA
ttggacttgcacagtaataaa

SEQ ID NO: 226 - RNA, 23 nt, synthetic construct
  source 1..23: mol_type = other RNA
tttattactgtgcaagtccaaat

SEQ ID NO: 227 - RNA, 19 nt, synthetic construct
  source 1..19: mol_type = other RNA
gttggtcacttctgtgcaa

SEQ ID NO: 228 - RNA, 21 nt, synthetic construct
  source 1..21: mol_type = other RNA
gggttggtcacttctgtgcaa

SEQ ID NO: 229 - RNA, 23 nt, synthetic construct
  source 1..23: mol_type = other RNA
ttgcacagaagtgaccaacccac

SEQ ID NO: 230 - RNA, 19 nt, synthetic construct
  source 1..19: mol_type = other RNA
gggattcagaaatgtggaa

SEQ ID NO: 231 - RNA, 21 nt, synthetic construct
  source 1..21: mol_type = other RNA
ttccacatttctgaatcccag

SEQ ID NO: 232 - RNA, 21 nt, synthetic construct
  source 1..21: mol_type = other RNA
ctgggattcagaaatgtggaa

SEQ ID NO: 233 - RNA, 23 nt, synthetic construct
  source 1..23: mol_type = other RNA
ttccacatttctgaatcccagga

SEQ ID NO: 234 - RNA, 19 nt, synthetic construct
  source 1..19: mol_type = other RNA
gatatgagcagcaatgata

SEQ ID NO: 235 - RNA, 21 nt, synthetic construct
  source 1..21: mol_type = other RNA
tagatatgagcagcaatgata

SEQ ID NO: 236 - RNA, 23 nt, synthetic construct
  source 1..23: mol_type = other RNA
tatcattgctgctcatatctaaa

SEQ ID NO: 237 - RNA, 19 nt, synthetic construct
  source 1..19: mol_type = other RNA
ggtcgtgaggaattctata

SEQ ID NO: 238 - RNA, 21 nt, synthetic construct
  source 1..21: mol_type = other RNA
caggtcgtgaggaattctata

SEQ ID NO: 239 - RNA, 23 nt, synthetic construct
  source 1..23: mol_type = other RNA
tatagaattcctcacgacctgca

SEQ ID NO: 240 - RNA, 19 nt, synthetic construct
  source 1..19: mol_type = other RNA
gatctgaagtcttagacaa

SEQ ID NO: 241 - RNA, 21 nt, synthetic construct
  source 1..21: mol_type = other RNA
aggatctgaagtcttagacaa

SEQ ID NO: 242 - RNA, 23 nt, synthetic construct
  source 1..23: mol_type = other RNA
ttgtctaagacttcagatcctac

SEQ ID NO: 243 - RNA, 19 nt, synthetic construct
  source 1..19: mol_type = other RNA
gatctcgtgaacaagatga

SEQ ID NO: 244 - RNA, 21 nt, synthetic construct
  source 1..21: mol_type = other RNA
tcatcttgttcacgagatcca

SEQ ID NO: 245 - RNA, 21 nt, synthetic construct
  source 1..21: mol_type = other RNA
tggatctcgtgaacaagatga

SEQ ID NO: 246 - RNA, 23 nt, synthetic construct
  source 1..23: mol_type = other RNA
tcatcttgttcacgagatccact

SEQ ID NO: 247 - RNA, 19 nt, synthetic construct
  source 1..19: mol_type = other RNA
ggatctgaagtcttagaca

SEQ ID NO: 248 - RNA, 21 nt, synthetic construct
  source 1..21: mol_type = other RNA
taggatctgaagtcttagaca

SEQ ID NO: 249 - RNA, 23 nt, synthetic construct
  source 1..23: mol_type = other RNA
tgtctaagacttcagatcctacc

SEQ ID NO: 250 - RNA, 19 nt, synthetic construct
  source 1..19: mol_type = other RNA
gccttattttccaatggga

SEQ ID NO: 251 - RNA, 21 nt, synthetic construct
  source 1..21: mol_type = other RNA
atgccttattttccaatggga

SEQ ID NO: 252 - RNA, 23 nt, synthetic construct
  source 1..23: mol_type = other RNA
tcccattggaaaataaggcattt

SEQ ID NO: 253 - RNA, 19 nt, synthetic construct
  source 1..19: mol_type = other RNA
gccataatcagatcagcaa

SEQ ID NO: 254 - RNA, 21 nt, synthetic construct
  source 1..21: mol_type = other RNA
ttgctgatctgattatggcta

SEQ ID NO: 255 - RNA, 21 nt, synthetic construct
  source 1..21: mol_type = other RNA
tagccataatcagatcagcaa

SEQ ID NO: 256 - RNA, 23 nt, synthetic construct
  source 1..23: mol_type = other RNA
ttgctgatctgattatggctaaa

SEQ ID NO: 257 - RNA, 19 nt, synthetic construct
  source 1..19: mol_type = other RNA
ggtgatcagatatcagata

SEQ ID NO: 258 - RNA, 21 nt, synthetic construct
  source 1..21: mol_type = other RNA
tatctgatatctgatcaccat

SEQ ID NO: 259 - RNA, 21 nt, synthetic construct
  source 1..21: mol_type = other RNA
atggtgatcagatatcagata

SEQ ID NO: 260 - RNA, 23 nt, synthetic construct
  source 1..23: mol_type = other RNA
tatctgatatctgatcaccattc

SEQ ID NO: 261 - RNA, 19 nt, synthetic construct
  source 1..19: mol_type = other RNA
tgacaagaatcctcacaat

SEQ ID NO: 262 - RNA, 21 nt, synthetic construct
  source 1..21: mol_type = other RNA
attgtgaggattcttgtcaac

SEQ ID NO: 263 - RNA, 19 nt, synthetic construct
  source 1..19: mol_type = other RNA
tgacaagaatcctcacaat

SEQ ID NO: 264 - RNA, 14 nt, synthetic construct
  source 1..14: mol_type = other RNA
  misc_feature 1: note = The G is attached to AUUGUG[GNA-A]
ggattcttgtcaac

SEQ ID NO: 265 - RNA, 19 nt, synthetic construct
  source 1..19: mol_type = other RNA
tgacaagaatcctcacaat

SEQ ID NO: 266 - RNA, 14 nt, synthetic construct
  source 1..14: mol_type = other RNA
  misc_feature 1: note = The G is attached to AUUGUG[N3T1-A]
ggattcttgtcaac

SEQ ID NO: 267 - RNA, 19 nt, synthetic construct
  source 1..19: mol_type = other RNA
tgacaagaatcctcacaat

SEQ ID NO: 268 - RNA, 16 nt, synthetic construct
  source 1..16: mol_type = other RNA
  misc_feature 1: note = The G is attached to AUUG[N3T1-U]
gaggattcttgtcaac

SEQ ID NO: 269 - DNA, 62 bp, synthetic construct
  source 1..62: mol_type = other DNA
gtcgcatataatattacataaataaaagtcgcatataatattacataaataaaagtcgcata

SEQ ID NO: 270 - DNA, 21 bp, synthetic construct
  source 1..21: mol_type = other DNA
gttgacaagaatcctcacaat

SEQ ID NO: 271 - DNA, 109 bp, synthetic construct
  source 1..109: mol_type = other DNA
  misc_feature 1..109: note = n: Special linker from Suzhou Biosyntech
tggtcacctccgactcacaatntggtcacctccgactcacaatntggtcacctccgactcacaatntggtcacctccgactcacaatntggtcacctccgactcacaat

SEQ ID NO: 272 - DNA, 21 bp, synthetic construct
  source 1..21: mol_type = other DNA
tgtgaatgcagaccaaagaaa

SEQ ID NO: 273 - DNA, 109 bp, synthetic construct
  source 1..109: mol_type = other DNA
  misc_feature 1..109: note = n: Special linker from Suzhou Biosyntech
gtgtccgtactcacaaagaaangtgtccgtactcacaaagaaangtgtccgtactcacaaagaaangtgtccgtactcacaaagaaangtgtccgtactcacaaagaaa

SEQ ID NO: 274 - RNA, 19 nt, synthetic construct
  source 1..19: mol_type = other RNA
agagaaaagagaaagtgtt

SEQ ID NO: 275 - RNA, 20 nt, synthetic construct
  source 1..20: mol_type = other RNA
  misc_feature 1: note = The A is attached to [N3T12-029-01]
acactttctcttttctcttt

SEQ ID NO: 276 - RNA, 21 nt, synthetic construct
  source 1..21: mol_type = other RNA
tgaatgcagaccaaagaaaga

SEQ ID NO: 277 - RNA, 22 nt, synthetic construct
  source 1..22: mol_type = other RNA
  misc_feature 1: note = The C is attached to [N3T12-029-01]
ctttctttggtctgcattcaca

SEQ ID NO: 278 - RNA, 19 nt, synthetic construct
  source 1..19: mol_type = other RNA
agagaaaagagaaagtgtt

SEQ ID NO: 279 - RNA, 20 nt, synthetic construct
  source 1..20: mol_type = other RNA
  misc_feature 1: note = The A is attached to [VPU]
acactttctcttttctcttt

SEQ ID NO: 280 - RNA, 21 nt, synthetic construct
  source 1..21: mol_type = other RNA
tgaatgcagaccaaagaaaga

SEQ ID NO: 281 - RNA, 22 nt, synthetic construct
  source 1..22: mol_type = other RNA
  misc_feature 1: note = The C is attached to [VPU]
ctttctttggtctgcattcaca

SEQ ID NO: 282 - RNA, 19 nt, synthetic construct
  source 1..19: mol_type = other RNA
agagaaaagagaaagtgtt

SEQ ID NO: 283 - RNA, 21 nt, synthetic construct
  source 1..21: mol_type = other RNA
aacactttctcttttctcttt

SEQ ID NO: 284 - RNA, 21 nt, synthetic construct
  source 1..21: mol_type = other RNA
tgaatgcagaccaaagaaaga

SEQ ID NO: 285 - RNA, 23 nt, synthetic construct
  source 1..23: mol_type = other RNA
tctttctttggtctgcattcaca

SEQ ID NO: 286 - RNA, 15 nt, synthetic construct
  source 1..15: mol_type = other RNA
  misc_feature 1: note = The Uf is attached to CmsUmsUmGmUm(Uhd)
  modified_base 13..15: mod_base = OTHER; note = phosphorothioate (PS) linkage
  modified_base 1: mod_base = OTHER; note = 2'-deoxy-2'-fluoro uridine
  modified_base 2: mod_base = gm
  modified_base 3: mod_base = OTHER; note = 2'-deoxy-2'-fluoro uridine
  modified_base 4: mod_base = OTHER; note = 2'-deoxy-2'-fluoro adenosine
  modified_base 5: mod_base = OTHER; note = 2'-deoxy-2'-fluoro uridine
  modified_base 6: mod_base = um
  modified_base 7: mod_base = gm
  modified_base 8: mod_base = cm
  modified_base 9: mod_base = OTHER; note = 2'-O-methyl adenosine
  modified_base 10: mod_base = OTHER; note = 2'-O-methyl adenosine
  modified_base 11: mod_base = um
  modified_base 12: mod_base = um
  modified_base 14: mod_base = cm
  modified_base 13: mod_base = cm
  modified_base 15: mod_base = OTHER; note = 2'-O-methyl adenosine
tgtattgcaattcca

SEQ ID NO: 287 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro guanosine
  modified_base 3: mod_base = gm
  modified_base 4: mod_base = OTHER; note = 2'-O-methyl adenosine
  modified_base 5: mod_base = OTHER; note = 2'-O-methyl adenosine
  modified_base 6: mod_base = OTHER; note = 2'-deoxy-2'-fluoro uridine
  modified_base 7: mod_base = um
  modified_base 8: mod_base = gm
  modified_base 9: mod_base = cm
  modified_base 10: mod_base = OTHER; note = 2'-O-methyl adenosine
  modified_base 11: mod_base = OTHER; note = 2'-O-methyl adenosine
  modified_base 12: mod_base = um
  modified_base 13: mod_base = OTHER; note = 2'-O-methyl adenosine
  modified_base 14: mod_base = OTHER; note = 2'-deoxy-2'-fluoro cytidine
  modified_base 15: mod_base = OTHER; note = 2'-O-methyl adenosine
  modified_base 16: mod_base = OTHER; note = 2'-deoxy-2'-fluoro adenosine
  modified_base 17: mod_base = OTHER; note = 2'-O-methyl adenosine
  modified_base 18: mod_base = cm
  modified_base 19: mod_base = OTHER; note = 2'-O-methyl adenosine
  modified_base 20: mod_base = OTHER; note = 2'-O-methyl adenosine
  modified_base 21: mod_base = gm
  modified_base 1..3: mod_base = OTHER; note = phosphorothioate (PS) linkage
  modified_base 21..23: mod_base = OTHER; note = phosphorothioate (PS) linkage
  modified_base 22: mod_base = um
  modified_base 23: mod_base = gm
tggaattgcaatacaaacaagtg

SEQ ID NO: 288 - RNA, 21 nt, synthetic construct
  source 1..21: mol_type = other RNA
  modified_base 1: mod_base = cm
  modified_base 2: mod_base = um
  modified_base 3: mod_base = um
  modified_base 4: mod_base = gm
  modified_base 5: mod_base = um
  modified_base 6: mod_base = um
  modified_base 7: mod_base = OTHER; note = 2'-deoxy-2'-fluoro uridine
  modified_base 8: mod_base = gm
  modified_base 9: mod_base = OTHER; note = 2'-deoxy-2'-fluoro uridine
  modified_base 10: mod_base = OTHER; note = 2'-deoxy-2'-fluoro adenosine
  modified_base 11: mod_base = OTHER; note = 2'-deoxy-2'-fluoro uridine
  modified_base 12: mod_base = um
  modified_base 13: mod_base = gm
  modified_base 14: mod_base = cm
  modified_base 15: mod_base = OTHER; note = 2'-O-methyl adenosine
  modified_base 16: mod_base = OTHER; note = 2'-O-methyl adenosine
  modified_base 17: mod_base = um
  modified_base 18: mod_base = um
  modified_base 19: mod_base = cm
  modified_base 20: mod_base = cm
  modified_base 21: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
cttgtttgtattgcaattcca

SEQ ID NO: 289 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro guanosine
  modified_base 3: mod_base = gm
  modified_base 4: mod_base = OTHER; note = 2'-O-methyl adenosine
  modified_base 5: mod_base = OTHER; note = 2'-O-methyl adenosine
  modified_base 6: mod_base = OTHER; note = 2'-deoxy-2'-fluoro uridine
  modified_base 7: mod_base = um
  modified_base 8: mod_base = gm
  modified_base 9: mod_base = cm
  modified_base 10: mod_base = OTHER; note = 2'-O-methyl adenosine
  modified_base 11: mod_base = OTHER; note = 2'-O-methyl adenosine
  modified_base 12: mod_base = um
  modified_base 13: mod_base = OTHER; note = 2'-O-methyl adenosine
  modified_base 14: mod_base = OTHER; note = 2'-deoxy-2'-fluoro cytidine
  modified_base 15: mod_base = OTHER; note = 2'-O-methyl adenosine
  modified_base 16: mod_base = OTHER; note = 2'-deoxy-2'-fluoro adenosine
  modified_base 17: mod_base = OTHER; note = 2'-O-methyl adenosine
  modified_base 18: mod_base = cm
  modified_base 19: mod_base = OTHER; note = 2'-O-methyl adenosine
  modified_base 20: mod_base = OTHER; note = 2'-O-methyl adenosine
  modified_base 21: mod_base = gm
  modified_base 1..3: mod_base = OTHER; note = phosphorothioate (PS) linkage
  modified_base 21..23: mod_base = OTHER; note = phosphorothioate (PS) linkage
  modified_base 22: mod_base = um
  modified_base 23: mod_base = gm
tggaattgcaatacaaacaagtg

SEQ ID NO: 290 - RNA, 21 nt, synthetic construct
  source 1..21: mol_type = other RNA
  modified_base 1: mod_base = gm
  modified_base 2: mod_base = um
  modified_base 3: mod_base = gm
  modified_base 4: mod_base = gm
  modified_base 5: mod_base = cm
  modified_base 6: mod_base = OTHER; note = 2'-O-methyl adenosine
  modified_base 7: mod_base = OTHER; note = 2'-deoxy-2'-fluoro adenosine
  modified_base 8: mod_base = um
  modified_base 9: mod_base = OTHER; note = 2'-deoxy-2'-fluoro guanosine
  modified_base 10: mod_base = OTHER; note = 2'-deoxy-2'-fluoro uridine
  modified_base 11: mod_base = OTHER; note = 2'-deoxy-2'-fluoro cytidine
  modified_base 12: mod_base = OTHER; note = 2'-O-methyl adenosine
  modified_base 13: mod_base = gm
  modified_base 14: mod_base = gm
  modified_base 15: mod_base = um
  modified_base 16: mod_base = gm
  modified_base 17: mod_base = um
  modified_base 18: mod_base = um
  modified_base 19: mod_base = OTHER; note = 2'-O-methyl adenosine
  modified_base 20: mod_base = um
  modified_base 21: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
gtggcaatgtcaggtgttata

SEQ ID NO: 291 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = um
  modified_base 4: mod_base = OTHER; note = 2'-O-methyl adenosine
  modified_base 5: mod_base = OTHER; note = 2'-O-methyl adenosine
  modified_base 6: mod_base = OTHER; note = 2'-deoxy-2'-fluoro cytidine
  modified_base 7: mod_base = OTHER; note = 2'-O-methyl adenosine
  modified_base 8: mod_base = cm
  modified_base 9: mod_base = cm
  modified_base 10: mod_base = um
  modified_base 11: mod_base = gm
  modified_base 12: mod_base = OTHER; note = 2'-O-methyl adenosine
  modified_base 13: mod_base = cm
  modified_base 14: mod_base = OTHER; note = 2'-deoxy-2'-fluoro adenosine
  modified_base 15: mod_base = um
  modified_base 16: mod_base = OTHER; note = 2'-deoxy-2'-fluoro uridine
  modified_base 17: mod_base = gm
  modified_base 18: mod_base = cm
  modified_base 19: mod_base = cm
  modified_base 20: mod_base = OTHER; note = 2'-O-methyl adenosine
  modified_base 21: mod_base = cm
  modified_base 1..3: mod_base = OTHER; note = phosphorothioate (PS) linkage
  modified_base 21..23: mod_base = OTHER; note = phosphorothioate (PS) linkage
  modified_base 22: mod_base = um
  modified_base 23: mod_base = cm
tataacacctgacattgccactc

SEQ ID NO: 292 - RNA, 21 nt, synthetic construct
  source 1..21: mol_type = other RNA
  modified_base 1: mod_base = um
  modified_base 2: mod_base = um
  modified_base 3: mod_base = gm
  modified_base 4: mod_base = gm
  modified_base 5: mod_base = OTHER; note = 2'-O-methyl adenosine
  modified_base 6: mod_base = cm
  modified_base 7: mod_base = OTHER; note = 2'-deoxy-2'-fluoro uridine
  modified_base 8: mod_base = um
  modified_base 9: mod_base = OTHER; note = 2'-deoxy-2'-fluoro guanosine
  modified_base 10: mod_base = OTHER; note = 2'-deoxy-2'-fluoro cytidine
  modified_base 11: mod_base = OTHER; note = 2'-deoxy-2'-fluoro adenosine
  modified_base 12: mod_base = cm
  modified_base 13: mod_base = OTHER; note = 2'-O-methyl adenosine
  modified_base 14: mod_base = gm
  modified_base 15: mod_base = um
  modified_base 16: mod_base = OTHER; note = 2'-O-methyl adenosine
  modified_base 17: mod_base = OTHER; note = 2'-O-methyl adenosine
  modified_base 18: mod_base = um
  modified_base 19: mod_base = OTHER; note = 2'-O-methyl adenosine
  modified_base 20: mod_base = OTHER; note = 2'-O-methyl adenosine
  modified_base 21: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
ttggacttgcacagtaataaa

SEQ ID NO: 293 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro uridine
  modified_base 3: mod_base = um
  modified_base 4: mod_base = OTHER; note = 2'-O-methyl adenosine
  modified_base 5: mod_base = um
  modified_base 6: mod_base = OTHER; note = 2'-deoxy-2'-fluoro uridine
  modified_base 7: mod_base = OTHER; note = 2'-O-methyl adenosine
  modified_base 8: mod_base = cm
  modified_base 9: mod_base = um
  modified_base 10: mod_base = gm
  modified_base 11: mod_base = um
  modified_base 12: mod_base = gm
  modified_base 13: mod_base = cm
  modified_base 14: mod_base = OTHER; note = 2'-deoxy-2'-fluoro adenosine
  modified_base 15: mod_base = OTHER; note = 2'-O-methyl adenosine
  modified_base 16: mod_base = OTHER; note = 2'-deoxy-2'-fluoro guanosine
  modified_base 17: mod_base = um
  modified_base 18: mod_base = cm
  modified_base 19: mod_base = cm
  modified_base 20: mod_base = OTHER; note = 2'-O-methyl adenosine
  modified_base 21: mod_base = OTHER; note = 2'-O-methyl adenosine
  modified_base 1..3: mod_base = OTHER; note = phosphorothioate (PS) linkage
  modified_base 21..23: mod_base = OTHER; note = phosphorothioate (PS) linkage
  modified_base 22: mod_base = OTHER; note = 2'-O-methyl adenosine
  modified_base 23: mod_base = um
tttattactgtgcaagtccaaat

SEQ ID NO: 294 - RNA, 21 nt, synthetic construct
  source 1..21: mol_type = other RNA
  modified_base 1: mod_base = gm
  modified_base 2: mod_base = gm
  modified_base 3: mod_base = gm
  modified_base 4: mod_base = um
  modified_base 5: mod_base = um
  modified_base 6: mod_base = gm
  modified_base 7: mod_base = OTHER; note = 2'-deoxy-2'-fluoro guanosine
  modified_base 8: mod_base = um
  modified_base 9: mod_base = OTHER; note = 2'-deoxy-2'-fluoro cytidine
  modified_base 10: mod_base = OTHER; note = 2'-deoxy-2'-fluoro adenosine
  modified_base 11: mod_base = OTHER; note = 2'-deoxy-2'-fluoro cytidine
  modified_base 12: mod_base = um
  modified_base 13: mod_base = um
  modified_base 14: mod_base = cm
  modified_base 15: mod_base = um
  modified_base 16: mod_base = gm
  modified_base 17: mod_base = um
  modified_base 18: mod_base = gm
  modified_base 19: mod_base = cm
  modified_base 20: mod_base = OTHER; note = 2'-O-methyl adenosine
  modified_base 21: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
gggttggtcacttctgtgcaa

SEQ ID NO: 295 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro uridine
  modified_base 3: mod_base = gm
  modified_base 4: mod_base = cm
  modified_base 5: mod_base = OTHER; note = 2'-O-methyl adenosine
  modified_base 6: mod_base = OTHER; note = 2'-deoxy-2'-fluoro cytidine
  modified_base 7: mod_base = OTHER; note = 2'-O-methyl adenosine
  modified_base 8: mod_base = gm
  modified_base 9: mod_base = OTHER; note = 2'-O-methyl adenosine
  modified_base 10: mod_base = OTHER; note = 2'-O-methyl adenosine
  modified_base 11: mod_base = gm
  modified_base 12: mod_base = um
  modified_base 13: mod_base = gm
  modified_base 14: mod_base = OTHER; note = 2'-deoxy-2'-fluoro adenosine
  modified_base 15: mod_base = cm
  modified_base 16: mod_base = OTHER; note = 2'-deoxy-2'-fluoro cytidine
  modified_base 17: mod_base = OTHER; note = 2'-O-methyl adenosine
  modified_base 18: mod_base = OTHER; note = 2'-O-methyl adenosine
  modified_base 19: mod_base = cm
  modified_base 20: mod_base = cm
  modified_base 21: mod_base = cm
  modified_base 1..3: mod_base = OTHER; note = phosphorothioate (PS) linkage
  modified_base 21..23: mod_base = OTHER; note = phosphorothioate (PS) linkage
  modified_base 22: mod_base = OTHER; note = 2'-O-methyl adenosine
  modified_base 23: mod_base = cm
ttgcacagaagtgaccaacccac

SEQ ID NO: 296 - RNA, 21 nt, synthetic construct
  source 1..21: mol_type = other RNA
  modified_base 1: mod_base = cm
  modified_base 2: mod_base = OTHER; note = 2'-O-methyl adenosine
  modified_base 3: mod_base = gm
  modified_base 4: mod_base = gm
  modified_base 5: mod_base = um
  modified_base 6: mod_base = cm
  modified_base 7: mod_base = OTHER; note = 2'-deoxy-2'-fluoro guanosine
  modified_base 8: mod_base = um
  modified_base 9: mod_base = OTHER; note = 2'-deoxy-2'-fluoro guanosine
  modified_base 10: mod_base = OTHER; note = 2'-deoxy-2'-fluoro adenosine
  modified_base 11: mod_base = OTHER; note = 2'-deoxy-2'-fluoro guanosine
  modified_base 12: mod_base = gm
  modified_base 13: mod_base = OTHER; note = 2'-O-methyl adenosine
  modified_base 14: mod_base = OTHER; note = 2'-O-methyl adenosine
  modified_base 15: mod_base = um
  modified_base 16: mod_base = um
  modified_base 17: mod_base = cm
  modified_base 18: mod_base = um
  modified_base 19: mod_base = OTHER; note = 2'-O-methyl adenosine
  modified_base 20: mod_base = um
  modified_base 21: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
caggtcgtgaggaattctata

SEQ ID NO: 297 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = um
  modified_base 4: mod_base = OTHER; note = 2'-O-methyl adenosine
  modified_base 5: mod_base = gm
  modified_base 6: mod_base = OTHER; note = 2'-deoxy-2'-fluoro adenosine
  modified_base 7: mod_base = OTHER; note = 2'-O-methyl adenosine
  modified_base 8: mod_base = um
  modified_base 9: mod_base = um
  modified_base 10: mod_base = cm
  modified_base 11: mod_base = cm
  modified_base 12: mod_base = um
  modified_base 13: mod_base = cm
  modified_base 14: mod_base = OTHER; note = 2'-deoxy-2'-fluoro adenosine
  modified_base 15: mod_base = cm
  modified_base 16: mod_base = OTHER; note = 2'-deoxy-2'-fluoro guanosine
  modified_base 17: mod_base = OTHER; note = 2'-O-methyl adenosine
  modified_base 18: mod_base = cm
  modified_base 19: mod_base = cm
  modified_base 20: mod_base = um
  modified_base 21: mod_base = gm
  modified_base 1..3: mod_base = OTHER; note = phosphorothioate (PS) linkage
  modified_base 21..23: mod_base = OTHER; note = phosphorothioate (PS) linkage
  modified_base 22: mod_base = cm
  modified_base 23: mod_base = OTHER; note = 2'-O-methyl adenosine
tatagaattcctcacgacctgca

SEQ ID NO: 298 - RNA, 21 nt, synthetic construct
  source 1..21: mol_type = other RNA
  modified_base 1: mod_base = um
  modified_base 2: mod_base = OTHER; note = 2'-O-methyl adenosine
  modified_base 3: mod_base = gm
  modified_base 4: mod_base = OTHER; note = 2'-O-methyl adenosine
  modified_base 5: mod_base = um
  modified_base 6: mod_base = OTHER; note = 2'-O-methyl adenosine
  modified_base 7: mod_base = OTHER; note = 2'-deoxy-2'-fluoro uridine
  modified_base 8: mod_base = gm
  modified_base 9: mod_base = OTHER; note = 2'-deoxy-2'-fluoro adenosine
  modified_base 10: mod_base = OTHER; note = 2'-deoxy-2'-fluoro guanosine
  modified_base 11: mod_base = OTHER; note = 2'-deoxy-2'-fluoro cytidine
  modified_base 12: mod_base = OTHER; note = 2'-O-methyl adenosine
  modified_base 13: mod_base = gm
  modified_base 14: mod_base = cm
  modified_base 15: mod_base = OTHER; note = 2'-O-methyl adenosine
  modified_base 16: mod_base = OTHER; note = 2'-O-methyl adenosine
  modified_base 17: mod_base = um
  modified_base 18: mod_base = gm
  modified_base 19: mod_base = OTHER; note = 2'-O-methyl adenosine
  modified_base 20: mod_base = um
  modified_base 21: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
tagatatgagcagcaatgata

SEQ ID NO: 299 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = um
  modified_base 4: mod_base = cm
  modified_base 5: mod_base = OTHER; note = 2'-O-methyl adenosine
  modified_base 6: mod_base = OTHER; note = 2'-deoxy-2'-fluoro uridine
  modified_base 7: mod_base = um
  modified_base 8: mod_base = gm
  modified_base 9: mod_base = cm
  modified_base 10: mod_base = um
  modified_base 11: mod_base = gm
  modified_base 12: mod_base = cm
  modified_base 13: mod_base = um
  modified_base 14: mod_base = OTHER; note = 2'-deoxy-2'-fluoro cytidine
  modified_base 15: mod_base = OTHER; note = 2'-O-methyl adenosine
  modified_base 16: mod_base = OTHER; note = 2'-deoxy-2'-fluoro uridine
  modified_base 17: mod_base = OTHER; note = 2'-O-methyl adenosine
  modified_base 18: mod_base = um
  modified_base 19: mod_base = cm
  modified_base 20: mod_base = um
  modified_base 21: mod_base = OTHER; note = 2'-O-methyl adenosine
  modified_base 1..3: mod_base = OTHER; note = phosphorothioate (PS) linkage
  modified_base 21..23: mod_base = OTHER; note = phosphorothioate (PS) linkage
  modified_base 22: mod_base = OTHER; note = 2'-O-methyl adenosine
  modified_base 23: mod_base = OTHER; note = 2'-O-methyl adenosine
tatcattgctgctcatatctaaa

SEQ ID NO: 300 - RNA, 21 nt, synthetic construct
  source 1..21: mol_type = other RNA
  modified_base 1: mod_base = cm
  modified_base 2: mod_base = um
  modified_base 3: mod_base = gm
  modified_base 4: mod_base = gm
  modified_base 5: mod_base = gm
  modified_base 6: mod_base = OTHER; note = 2'-O-methyl adenosine
  modified_base 7: mod_base = OTHER; note = 2'-deoxy-2'-fluoro uridine
  modified_base 8: mod_base = um
  modified_base 9: mod_base = OTHER; note = 2'-deoxy-2'-fluoro cytidine
  modified_base 10: mod_base = OTHER; note = 2'-deoxy-2'-fluoro adenosine
  modified_base 11: mod_base = OTHER; note = 2'-deoxy-2'-fluoro guanosine
  modified_base 12: mod_base = OTHER; note = 2'-O-methyl adenosine
  modified_base 13: mod_base = OTHER; note = 2'-O-methyl adenosine
  modified_base 14: mod_base = OTHER; note = 2'-O-methyl adenosine
  modified_base 15: mod_base = um
  modified_base 16: mod_base = gm
  modified_base 17: mod_base = um
  modified_base 18: mod_base = gm
  modified_base 19: mod_base = gm
  modified_base 20: mod_base = OTHER; note = 2'-O-methyl adenosine
  modified_base 21: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
ctgggattcagaaatgtggaa

SEQ ID NO: 301 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro uridine
  modified_base 3: mod_base = cm
  modified_base 4: mod_base = cm
  modified_base 5: mod_base = OTHER; note = 2'-O-methyl adenosine
  modified_base 6: mod_base = OTHER; note = 2'-deoxy-2'-fluoro cytidine
  modified_base 7: mod_base = OTHER; note = 2'-O-methyl adenosine
  modified_base 8: mod_base = um
  modified_base 9: mod_base = um
  modified_base 10: mod_base = um
  modified_base 11: mod_base = cm
  modified_base 12: mod_base = um
  modified_base 13: mod_base = gm
  modified_base 14: mod_base = OTHER; note = 2'-deoxy-2'-fluoro adenosine
  modified_base 15: mod_base = OTHER; note = 2'-O-methyl adenosine
  modified_base 16: mod_base = OTHER; note = 2'-deoxy-2'-fluoro uridine
  modified_base 17: mod_base = cm
  modified_base 18: mod_base = cm
  modified_base 19: mod_base = cm
  modified_base 20: mod_base = OTHER; note = 2'-O-methyl adenosine
  modified_base 21: mod_base = gm
  modified_base 1..3: mod_base = OTHER; note = phosphorothioate (PS) linkage
  modified_base 21..23: mod_base = OTHER; note = phosphorothioate (PS) linkage
  modified_base 22: mod_base = gm
  modified_base 23: mod_base = OTHER; note = 2'-O-methyl adenosine
ttccacatttctgaatcccagga

SEQ ID NO: 302 - RNA, 21 nt, synthetic construct
  source 1..21: mol_type = other RNA
  modified_base 1: mod_base = OTHER; note = 2'-O-methyl adenosine
  modified_base 2: mod_base = gm
  modified_base 3: mod_base = gm
  modified_base 4: mod_base = OTHER; note = 2'-O-methyl adenosine
  modified_base 5: mod_base = um
  modified_base 6: mod_base = cm
  modified_base 7: mod_base = OTHER; note = 2'-deoxy-2'-fluoro uridine
  modified_base 8: mod_base = gm
  modified_base 9: mod_base = OTHER; note = 2'-deoxy-2'-fluoro adenosine
  modified_base 10: mod_base = OTHER; note = 2'-deoxy-2'-fluoro adenosine
  modified_base 11: mod_base = OTHER; note = 2'-deoxy-2'-fluoro guanosine
  modified_base 12: mod_base = um
  modified_base 13: mod_base = cm
  modified_base 14: mod_base = um
  modified_base 15: mod_base = um
  modified_base 16: mod_base = OTHER; note = 2'-O-methyl adenosine
  modified_base 17: mod_base = gm
  modified_base 18: mod_base = OTHER; note = 2'-O-methyl adenosine
  modified_base 19: mod_base = cm
  modified_base 20: mod_base = OTHER; note = 2'-O-methyl adenosine
  modified_base 21: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
aggatctgaagtcttagacaa

SEQ ID NO: 303 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro uridine
  modified_base 3: mod_base = gm
  modified_base 4: mod_base = um
  modified_base 5: mod_base = cm
  modified_base 6: mod_base = OTHER; note = 2'-deoxy-2'-fluoro uridine
  modified_base 7: mod_base = OTHER; note = 2'-O-methyl adenosine
  modified_base 8: mod_base = OTHER; note = 2'-O-methyl adenosine
  modified_base 9: mod_base = gm
  modified_base 10: mod_base = OTHER; note = 2'-O-methyl adenosine
  modified_base 11: mod_base = cm
  modified_base 12: mod_base = um
  modified_base 13: mod_base = um
  modified_base 14: mod_base = OTHER; note = 2'-deoxy-2'-fluoro cytidine
  modified_base 15: mod_base = OTHER; note = 2'-O-methyl adenosine
  modified_base 16: mod_base = OTHER; note = 2'-deoxy-2'-fluoro guanosine
  modified_base 17: mod_base = OTHER; note = 2'-O-methyl adenosine
  modified_base 18: mod_base = um
  modified_base 19: mod_base = cm
  modified_base 20: mod_base = cm
  modified_base 21: mod_base = um
  modified_base 1..3: mod_base = OTHER; note = phosphorothioate (PS) linkage
  modified_base 21..23: mod_base = OTHER; note = phosphorothioate (PS) linkage
  modified_base 22: mod_base = OTHER; note = 2'-O-methyl adenosine
  modified_base 23: mod_base = cm
ttgtctaagacttcagatcctac

SEQ ID NO: 304 - RNA, 21 nt, synthetic construct
  source 1..21: mol_type = other RNA
  modified_base 1: mod_base = um
  modified_base 2: mod_base = gm
  modified_base 3: mod_base = gm
  modified_base 4: mod_base = OTHER; note = 2'-O-methyl adenosine
  modified_base 5: mod_base = um
  modified_base 6: mod_base = cm
  modified_base 7: mod_base = OTHER; note = 2'-deoxy-2'-fluoro uridine
  modified_base 8: mod_base = cm
  modified_base 9: mod_base = OTHER; note = 2'-deoxy-2'-fluoro guanosine
  modified_base 10: mod_base = OTHER; note = 2'-deoxy-2'-fluoro uridine
  modified_base 11: mod_base = OTHER; note = 2'-deoxy-2'-fluoro guanosine
  modified_base 12: mod_base = OTHER; note = 2'-O-methyl adenosine
  modified_base 13: mod_base = OTHER; note = 2'-O-methyl adenosine
  modified_base 14: mod_base = cm
  modified_base 15: mod_base = OTHER; note = 2'-O-methyl adenosine
  modified_base 16: mod_base = OTHER; note = 2'-O-methyl adenosine
  modified_base 17: mod_base = gm
  modified_base 18: mod_base = OTHER; note = 2'-O-methyl adenosine
  modified_base 19: mod_base = um
  modified_base 20: mod_base = gm
  modified_base 21: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
tggatctcgtgaacaagatga

SEQ ID NO: 305 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro cytidine
  modified_base 3: mod_base = OTHER; note = 2'-O-methyl adenosine
  modified_base 4: mod_base = um
  modified_base 5: mod_base = cm
  modified_base 6: mod_base = OTHER; note = 2'-deoxy-2'-fluoro uridine
  modified_base 7: mod_base = um
  modified_base 8: mod_base = gm
  modified_base 9: mod_base = um
  modified_base 10: mod_base = um
  modified_base 11: mod_base = cm
  modified_base 12: mod_base = OTHER; note = 2'-O-methyl adenosine
  modified_base 13: mod_base = cm
  modified_base 14: mod_base = OTHER; note = 2'-deoxy-2'-fluoro guanosine
  modified_base 15: mod_base = OTHER; note = 2'-O-methyl adenosine
  modified_base 16: mod_base = OTHER; note = 2'-deoxy-2'-fluoro guanosine
  modified_base 17: mod_base = OTHER; note = 2'-O-methyl adenosine
  modified_base 18: mod_base = um
  modified_base 19: mod_base = cm
  modified_base 20: mod_base = cm
  modified_base 21: mod_base = OTHER; note = 2'-O-methyl adenosine
  modified_base 1..3: mod_base = OTHER; note = phosphorothioate (PS) linkage
  modified_base 21..23: mod_base = OTHER; note = phosphorothioate (PS) linkage
  modified_base 22: mod_base = cm
  modified_base 23: mod_base = um
tcatcttgttcacgagatccact

SEQ ID NO: 306 - RNA, 21 nt, synthetic construct
  source 1..21: mol_type = other RNA
  modified_base 1: mod_base = um
  modified_base 2: mod_base = OTHER; note = 2'-O-methyl adenosine
  modified_base 3: mod_base = gm
  modified_base 4: mod_base = cm
  modified_base 5: mod_base = cm
  modified_base 6: mod_base = OTHER; note = 2'-O-methyl adenosine
  modified_base 7: mod_base = OTHER; note = 2'-deoxy-2'-fluoro uridine
  modified_base 8: mod_base = OTHER; note = 2'-O-methyl adenosine
  modified_base 9: mod_base = OTHER; note = 2'-deoxy-2'-fluoro adenosine
  modified_base 10: mod_base = OTHER; note = 2'-deoxy-2'-fluoro uridine
  modified_base 11: mod_base = OTHER; note = 2'-deoxy-2'-fluoro cytidine
  modified_base 12: mod_base = OTHER; note = 2'-O-methyl adenosine
  modified_base 13: mod_base = gm
  modified_base 14: mod_base = OTHER; note = 2'-O-methyl adenosine
  modified_base 15: mod_base = um
  modified_base 16: mod_base = cm
  modified_base 17: mod_base = OTHER; note = 2'-O-methyl adenosine
  modified_base 18: mod_base = gm
  modified_base 19: mod_base = cm
  modified_base 20: mod_base = OTHER; note = 2'-O-methyl adenosine
  modified_base 21: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
tagccataatcagatcagcaa

SEQ ID NO: 307 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro uridine
  modified_base 3: mod_base = gm
  modified_base 4: mod_base = cm
  modified_base 5: mod_base = um
  modified_base 6: mod_base = OTHER; note = 2'-deoxy-2'-fluoro guanosine
  modified_base 7: mod_base = OTHER; note = 2'-O-methyl adenosine
  modified_base 8: mod_base = um
  modified_base 9: mod_base = cm
  modified_base 10: mod_base = um
  modified_base 11: mod_base = gm
  modified_base 12: mod_base = OTHER; note = 2'-O-methyl adenosine
  modified_base 13: mod_base = um
  modified_base 14: mod_base = OTHER; note = 2'-deoxy-2'-fluoro uridine
  modified_base 15: mod_base = OTHER; note = 2'-O-methyl adenosine
  modified_base 16: mod_base = OTHER; note = 2'-deoxy-2'-fluoro uridine
  modified_base 17: mod_base = gm
  modified_base 18: mod_base = gm
  modified_base 19: mod_base = cm
  modified_base 20: mod_base = um
  modified_base 21: mod_base = OTHER; note = 2'-O-methyl adenosine
  modified_base 1..3: mod_base = OTHER; note = phosphorothioate (PS) linkage
  modified_base 21..23: mod_base = OTHER; note = phosphorothioate (PS) linkage
  modified_base 22: mod_base = OTHER; note = 2'-O-methyl adenosine
  modified_base 23: mod_base = OTHER; note = 2'-O-methyl adenosine
ttgctgatctgattatggctaaa

SEQ ID NO: 308 - RNA, 21 nt, synthetic construct
  source 1..21: mol_type = other RNA
  modified_base 1: mod_base = OTHER; note = 2'-O-methyl adenosine
  modified_base 2: mod_base = um
  modified_base 3: mod_base = gm
  modified_base 4: mod_base = gm
  modified_base 5: mod_base = um
  modified_base 6: mod_base = gm
  modified_base 7: mod_base = OTHER; note = 2'-deoxy-2'-fluoro adenosine
  modified_base 8: mod_base = um
  modified_base 9: mod_base = OTHER; note = 2'-deoxy-2'-fluoro cytidine
  modified_base 10: mod_base = OTHER; note = 2'-deoxy-2'-fluoro adenosine
  modified_base 11: mod_base = OTHER; note = 2'-deoxy-2'-fluoro guanosine
  modified_base 12: mod_base = OTHER; note = 2'-O-methyl adenosine
  modified_base 13: mod_base = um
  modified_base 14: mod_base = OTHER; note = 2'-O-methyl adenosine
  modified_base 15: mod_base = um
  modified_base 16: mod_base = cm
  modified_base 17: mod_base = OTHER; note = 2'-O-methyl adenosine
  modified_base 18: mod_base = gm
  modified_base 19: mod_base = OTHER; note = 2'-O-methyl adenosine
  modified_base 20: mod_base = um
  modified_base 21: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
atggtgatcagatatcagata

SEQ ID NO: 309 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = um
  modified_base 4: mod_base = cm
  modified_base 5: mod_base = um
  modified_base 6: mod_base = OTHER; note = 2'-deoxy-2'-fluoro guanosine
  modified_base 7: mod_base = OTHER; note = 2'-O-methyl adenosine
  modified_base 8: mod_base = um
  modified_base 9: mod_base = OTHER; note = 2'-O-methyl adenosine
  modified_base 10: mod_base = um
  modified_base 11: mod_base = cm
  modified_base 12: mod_base = um
  modified_base 13: mod_base = gm
  modified_base 14: mod_base = OTHER; note = 2'-deoxy-2'-fluoro adenosine
  modified_base 15: mod_base = um
  modified_base 16: mod_base = OTHER; note = 2'-deoxy-2'-fluoro cytidine
  modified_base 17: mod_base = OTHER; note = 2'-O-methyl adenosine
  modified_base 18: mod_base = cm
  modified_base 19: mod_base = cm
  modified_base 20: mod_base = OTHER; note = 2'-O-methyl adenosine
  modified_base 21: mod_base = um
  modified_base 1..3: mod_base = OTHER; note = phosphorothioate (PS) linkage
  modified_base 21..23: mod_base = OTHER; note = phosphorothioate (PS) linkage
  modified_base 22: mod_base = um
  modified_base 23: mod_base = cm
tatctgatatctgatcaccattc

SEQ ID NO: 310 - RNA, 15 nt, synthetic construct
  source 1..15: mol_type = other RNA
  modified_base 1: mod_base = OTHER; note = 2'-deoxy-2'-fluoro uridine
  modified_base 2: mod_base = gm
  modified_base 3: mod_base = OTHER; note = 2'-deoxy-2'-fluoro uridine
  modified_base 4: mod_base = OTHER; note = 2'-deoxy-2'-fluoro adenosine
  modified_base 5: mod_base = OTHER; note = 2'-deoxy-2'-fluoro uridine
  modified_base 6: mod_base = um
  modified_base 7: mod_base = gm
  modified_base 8: mod_base = cm
  modified_base 9: mod_base = OTHER; note = 2'-O-methyl adenosine
  modified_base 10: mod_base = OTHER; note = 2'-O-methyl adenosine
  modified_base 11: mod_base = um
  modified_base 12: mod_base = um
  modified_base 13: mod_base = cm
  modified_base 14: mod_base = cm
  modified_base 15: mod_base = OTHER; note = 2'-O-methyl adenosine
  modified_base 13..15: mod_base = OTHER; note = phosphorothioate (PS) linkage
  misc_feature 1: note = The Uf is attached to CmsUmsUmGmUm(Uhd)
tgtattgcaattcca

SEQ ID NO: 311 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro guanosine
  modified_base 3: mod_base = gm
  modified_base 4: mod_base = OTHER; note = 2'-O-methyl adenosine
  modified_base 5: mod_base = OTHER; note = 2'-O-methyl adenosine
  modified_base 6: mod_base = OTHER; note = 2'-deoxy-2'-fluoro uridine
  modified_base 7: mod_base = um
  modified_base 8: mod_base = gm
  modified_base 9: mod_base = cm
  modified_base 10: mod_base = OTHER; note = 2'-O-methyl adenosine
  modified_base 11: mod_base = OTHER; note = 2'-O-methyl adenosine
  modified_base 12: mod_base = um
  modified_base 13: mod_base = OTHER; note = 2'-O-methyl adenosine
  modified_base 14: mod_base = OTHER; note = 2'-deoxy-2'-fluoro cytidine
  modified_base 15: mod_base = OTHER; note = 2'-O-methyl adenosine
  modified_base 16: mod_base = OTHER; note = 2'-deoxy-2'-fluoro adenosine
  modified_base 17: mod_base = OTHER; note = 2'-O-methyl adenosine
  modified_base 18: mod_base = cm
  modified_base 19: mod_base = OTHER; note = 2'-O-methyl adenosine
  modified_base 20: mod_base = OTHER; note = 2'-O-methyl adenosine
  modified_base 21: mod_base = gm
  modified_base 1..3: mod_base = OTHER; note = phosphorothioate (PS) linkage
  modified_base 21..23: mod_base = OTHER; note = phosphorothioate (PS) linkage
  modified_base 22: mod_base = um
  modified_base 23: mod_base = gm
tggaattgcaatacaaacaagtg

SEQ ID NO: 312 - RNA, 21 nt, synthetic construct
  source 1..21: mol_type = other RNA
  modified_base 7: mod_base = OTHER; note = 2'-deoxy-2'-fluoro uridine
  modified_base 8: mod_base = gm
  modified_base 9: mod_base = OTHER; note = 2'-deoxy-2'-fluoro uridine
  modified_base 10: mod_base = OTHER; note = 2'-deoxy-2'-fluoro adenosine
  modified_base 11: mod_base = OTHER; note = 2'-deoxy-2'-fluoro uridine
  modified_base 12: mod_base = um
  modified_base 13: mod_base = gm
  modified_base 14: mod_base = cm
  modified_base 15: mod_base = OTHER; note = 2'-O-methyl adenosine
  modified_base 16: mod_base = OTHER; note = 2'-O-methyl adenosine
  modified_base 17: mod_base = um
  modified_base 18: mod_base = um
  modified_base 19: mod_base = cm
  modified_base 20: mod_base = cm
  modified_base 21: mod_base = OTHER; note = 2'-O-methyl adenosine
  modified_base 20^21: mod_base = OTHER; note = phosphorothioate (PS) linkage
  modified_base 1^2: mod_base = OTHER; note = phosphorothioate (PS) linkage
  misc_feature 21: note = The Am is attached through the phosphorothioate linkage to [N3T12-012-01]
  modified_base 1..3: mod_base = OTHER; note = phosphorothioate (PS) linkage
  modified_base 1: mod_base = cm
  modified_base 2: mod_base = um
  modified_base 3: mod_base = um
  modified_base 4: mod_base = gm
  modified_base 5: mod_base = um
  modified_base 6: mod_base = um
cttgtttgtattgcaattcca

SEQ ID NO: 313 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro guanosine
  modified_base 3: mod_base = gm
  modified_base 4: mod_base = OTHER; note = 2'-O-methyl adenosine
  modified_base 5: mod_base = OTHER; note = 2'-O-methyl adenosine
  modified_base 6: mod_base = OTHER; note = 2'-deoxy-2'-fluoro uridine
  modified_base 7: mod_base = um
  modified_base 8: mod_base = gm
  modified_base 9: mod_base = cm
  modified_base 10: mod_base = OTHER; note = 2'-O-methyl adenosine
  modified_base 11: mod_base = OTHER; note = 2'-O-methyl adenosine
  modified_base 12: mod_base = um
  modified_base 13: mod_base = OTHER; note = 2'-O-methyl adenosine
  modified_base 14: mod_base = OTHER; note = 2'-deoxy-2'-fluoro cytidine
  modified_base 15: mod_base = OTHER; note = 2'-O-methyl adenosine
  modified_base 16: mod_base = OTHER; note = 2'-deoxy-2'-fluoro adenosine
  modified_base 17: mod_base = OTHER; note = 2'-O-methyl adenosine
  modified_base 18: mod_base = cm
  modified_base 19: mod_base = OTHER; note = 2'-O-methyl adenosine
  modified_base 20: mod_base = OTHER; note = 2'-O-methyl adenosine
  modified_base 21: mod_base = gm
  modified_base 1..3: mod_base = OTHER; note = phosphorothioate (PS) linkage
  modified_base 21..23: mod_base = OTHER; note = phosphorothioate (PS) linkage
  modified_base 22: mod_base = um
  modified_base 23: mod_base = gm
tggaattgcaatacaaacaagtg

SEQ ID NO: 314 - RNA, 21 nt, synthetic construct
  source 1..21: mol_type = other RNA
  modified_base 1: mod_base = gm
  modified_base 2: mod_base = um
  modified_base 3: mod_base = gm
  modified_base 4: mod_base = gm
  modified_base 5: mod_base = cm
  modified_base 6: mod_base = OTHER; note = 2'-O-methyl adenosine
  modified_base 7: mod_base = OTHER; note = 2'-deoxy-2'-fluoro adenosine
  modified_base 8: mod_base = um
  modified_base 9: mod_base = OTHER; note = 2'-deoxy-2'-fluoro guanosine
  modified_base 10: mod_base = OTHER; note = 2'-deoxy-2'-fluoro uridine
  modified_base 11: mod_base = OTHER; note = 2'-deoxy-2'-fluoro cytidine
  modified_base 12: mod_base = OTHER; note = 2'-O-methyl adenosine
  modified_base 13: mod_base = gm
  modified_base 14: mod_base = gm
  modified_base 15: mod_base = um
  modified_base 16: mod_base = gm
  modified_base 17: mod_base = um
  modified_base 18: mod_base = um
  modified_base 19: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
  modified_base 20: mod_base = um
  modified_base 21: mod_base = OTHER; note = 2'-O-methyl adenosine
  modified_base 7^8: mod_base = OTHER; note = phosphorothioate (PS) linkage
gtggcaatgtcaggtgttata

SEQ ID NO: 315 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = um
  modified_base 4: mod_base = OTHER; note = 2'-O-methyl adenosine
  modified_base 5: mod_base = OTHER; note = 2'-O-methyl adenosine
  modified_base 6: mod_base = OTHER; note = 2'-deoxy-2'-fluoro cytidine
  modified_base 7: mod_base = OTHER; note = 2'-O-methyl adenosine
  modified_base 8: mod_base = cm
  modified_base 9: mod_base = cm
  modified_base 10: mod_base = um
  modified_base 11: mod_base = gm
  modified_base 12: mod_base = OTHER; note = 2'-O-methyl adenosine
  modified_base 13: mod_base = cm
  modified_base 14: mod_base = OTHER; note = 2'-deoxy-2'-fluoro adenosine
  modified_base 15: mod_base = um
  modified_base 16: mod_base = OTHER; note = 2'-deoxy-2'-fluoro uridine
  modified_base 17: mod_base = gm
  modified_base 18: mod_base = cm
  modified_base 19: mod_base = cm
  modified_base 20: mod_base = OTHER; note = 2'-O-methyl adenosine
  modified_base 21: mod_base = cm
  modified_base 1..3: mod_base = OTHER; note = phosphorothioate (PS) linkage
  modified_base 21..23: mod_base = OTHER; note = phosphorothioate (PS) linkage
  modified_base 22: mod_base = um
  modified_base 23: mod_base = cm
tataacacctgacattgccactc

SEQ ID NO: 316 - RNA, 21 nt, synthetic construct
  source 1..21: mol_type = other RNA
  modified_base 1: mod_base = gm
  modified_base 2: mod_base = um
  modified_base 3: mod_base = gm
  modified_base 4: mod_base = gm
  modified_base 5: mod_base = cm
  modified_base 6: mod_base = OTHER; note = 2'-O-methyl adenosine
  modified_base 7: mod_base = OTHER; note = 2'-deoxy-2'-fluoro adenosine
  modified_base 8: mod_base = um
  modified_base 9: mod_base = OTHER; note = 2'-deoxy-2'-fluoro guanosine
  modified_base 10: mod_base = OTHER; note = 2'-deoxy-2'-fluoro uridine
  modified_base 11: mod_base = OTHER; note = 2'-deoxy-2'-fluoro cytidine
  modified_base 12: mod_base = OTHER; note = 2'-O-methyl adenosine
  modified_base 13: mod_base = gm
  modified_base 14: mod_base = gm
  modified_base 15: mod_base = um
  modified_base 16: mod_base = gm
  modified_base 17: mod_base = um
  modified_base 18: mod_base = um
  modified_base 19: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
  modified_base 20: mod_base = um
  modified_base 21: mod_base = OTHER; note = 2'-O-methyl adenosine
  modified_base 7^8: mod_base = OTHER; note = phosphorothioate (PS) linkage
gtggcaatgtcaggtgttata

SEQ ID NO: 317 - RNA, 18 nt, synthetic construct
  source 1..18: mol_type = other RNA
  modified_base 1: mod_base = OTHER; note = 2'-deoxy-2'-fluoro cytidine
  modified_base 2: mod_base = OTHER; note = 2'-O-methyl adenosine
  modified_base 3: mod_base = cm
  modified_base 4: mod_base = cm
  modified_base 5: mod_base = um
  modified_base 6: mod_base = gm
  modified_base 7: mod_base = OTHER; note = 2'-O-methyl adenosine
  modified_base 8: mod_base = cm
  modified_base 9: mod_base = OTHER; note = 2'-deoxy-2'-fluoro adenosine
  modified_base 10: mod_base = um
  modified_base 11: mod_base = OTHER; note = 2'-deoxy-2'-fluoro uridine
  modified_base 12: mod_base = gm
  modified_base 13: mod_base = cm
  modified_base 14: mod_base = cm
  modified_base 15: mod_base = OTHER; note = 2'-O-methyl adenosine
  modified_base 16: mod_base = cm
  misc_feature 1: note = The Cf is attached to VPUmsAfsUmAm[N3T1-A-R]
  modified_base 16..18: mod_base = OTHER; note = phosphorothioate (PS) linkage
  modified_base 17: mod_base = um
  modified_base 18: mod_base = cm
cacctgacattgccactc

SEQ ID NO: 318 - RNA, 21 nt, synthetic construct
  source 1..21: mol_type = other RNA
  modified_base 1: mod_base = gm
  modified_base 2: mod_base = um
  modified_base 3: mod_base = gm
  modified_base 4: mod_base = gm
  modified_base 5: mod_base = cm
  modified_base 6: mod_base = OTHER; note = 2'-O-methyl adenosine
  modified_base 7: mod_base = OTHER; note = 2'-deoxy-2'-fluoro adenosine
  modified_base 8: mod_base = um
  modified_base 9: mod_base = OTHER; note = 2'-deoxy-2'-fluoro guanosine
  modified_base 10: mod_base = OTHER; note = 2'-deoxy-2'-fluoro uridine
  modified_base 11: mod_base = OTHER; note = 2'-deoxy-2'-fluoro cytidine
  modified_base 12: mod_base = OTHER; note = 2'-O-methyl adenosine
  modified_base 13: mod_base = gm
  modified_base 14: mod_base = gm
  modified_base 15: mod_base = um
  modified_base 16: mod_base = gm
  modified_base 17: mod_base = um
  modified_base 18: mod_base = um
  modified_base 19: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
  modified_base 20: mod_base = um
  modified_base 21: mod_base = OTHER; note = 2'-O-methyl adenosine
  modified_base 7^8: mod_base = OTHER; note = phosphorothioate (PS) linkage
gtggcaatgtcaggtgttata

SEQ ID NO: 319 - RNA, 16 nt, synthetic construct
  source 1..16: mol_type = other RNA
  modified_base 1: mod_base = cm
  modified_base 2: mod_base = cm
  modified_base 3: mod_base = um
  modified_base 4: mod_base = gm
  modified_base 5: mod_base = OTHER; note = 2'-O-methyl adenosine
  modified_base 6: mod_base = cm
  modified_base 7: mod_base = OTHER; note = 2'-deoxy-2'-fluoro adenosine
  modified_base 8: mod_base = um
  modified_base 9: mod_base = OTHER; note = 2'-deoxy-2'-fluoro uridine
  modified_base 10: mod_base = gm
  modified_base 11: mod_base = cm
  modified_base 12: mod_base = cm
  modified_base 13: mod_base = OTHER; note = 2'-O-methyl adenosine
  modified_base 14: mod_base = cm
  misc_feature 1: note = The Cm is attached to VPUmsAfsUmAmAmCf[N3T1-A-R]
  modified_base 14..16: mod_base = OTHER; note = phosphorothioate (PS) linkage
  modified_base 15: mod_base = um
  modified_base 16: mod_base = cm
cctgacattgccactc

SEQ ID NO: 320 - RNA, 21 nt, synthetic construct
  source 1..21: mol_type = other RNA
  modified_base 1: mod_base = um
  modified_base 2: mod_base = um
  modified_base 3: mod_base = gm
  modified_base 4: mod_base = gm
  modified_base 5: mod_base = OTHER; note = 2'-O-methyl adenosine
  modified_base 6: mod_base = cm
  modified_base 7: mod_base = OTHER; note = 2'-deoxy-2'-fluoro uridine
  modified_base 8: mod_base = um
  modified_base 9: mod_base = OTHER; note = 2'-deoxy-2'-fluoro guanosine
  modified_base 10: mod_base = OTHER; note = 2'-deoxy-2'-fluoro cytidine
  modified_base 11: mod_base = OTHER; note = 2'-deoxy-2'-fluoro adenosine
  modified_base 12: mod_base = cm
  modified_base 13: mod_base = OTHER; note = 2'-O-methyl adenosine
  modified_base 14: mod_base = gm
  modified_base 15: mod_base = um
  modified_base 16: mod_base = OTHER; note = 2'-O-methyl adenosine
  modified_base 17: mod_base = OTHER; note = 2'-O-methyl adenosine
  modified_base 18: mod_base = um
  modified_base 19: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
  modified_base 20: mod_base = OTHER; note = 2'-O-methyl adenosine
  modified_base 21: mod_base = OTHER; note = 2'-O-methyl adenosine
ttggacttgcacagtaataaa

SEQ ID NO: 321 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro uridine
  modified_base 3: mod_base = um
  modified_base 4: mod_base = OTHER; note = 2'-O-methyl adenosine
  modified_base 5: mod_base = um
  modified_base 6: mod_base = OTHER; note = 2'-deoxy-2'-fluoro uridine
  modified_base 7: mod_base = OTHER; note = 2'-O-methyl adenosine
  modified_base 8: mod_base = cm
  modified_base 9: mod_base = um
  modified_base 10: mod_base = gm
  modified_base 11: mod_base = um
  modified_base 12: mod_base = gm
  modified_base 13: mod_base = cm
  modified_base 14: mod_base = OTHER; note = 2'-deoxy-2'-fluoro adenosine
  modified_base 15: mod_base = OTHER; note = 2'-O-methyl adenosine
  modified_base 16: mod_base = OTHER; note = 2'-deoxy-2'-fluoro guanosine
  modified_base 17: mod_base = um
  modified_base 18: mod_base = cm
  modified_base 19: mod_base = cm
  modified_base 20: mod_base = OTHER; note = 2'-O-methyl adenosine
  modified_base 21: mod_base = OTHER; note = 2'-O-methyl adenosine
  modified_base 1..3: mod_base = OTHER; note = phosphorothioate (PS) linkage
  modified_base 21..23: mod_base = OTHER; note = phosphorothioate (PS) linkage
  modified_base 22: mod_base = OTHER; note = 2'-O-methyl adenosine
  modified_base 23: mod_base = um
tttattactgtgcaagtccaaat

SEQ ID NO: 322 - RNA, 21 nt, synthetic construct
  source 1..21: mol_type = other RNA
  modified_base 1: mod_base = um
  modified_base 2: mod_base = um
  modified_base 3: mod_base = gm
  modified_base 4: mod_base = gm
  modified_base 5: mod_base = OTHER; note = 2'-O-methyl adenosine
  modified_base 6: mod_base = cm
  modified_base 7: mod_base = OTHER; note = 2'-deoxy-2'-fluoro uridine
  modified_base 8: mod_base = um
  modified_base 9: mod_base = OTHER; note = 2'-deoxy-2'-fluoro guanosine
  modified_base 10: mod_base = OTHER; note = 2'-deoxy-2'-fluoro cytidine
  modified_base 11: mod_base = OTHER; note = 2'-deoxy-2'-fluoro adenosine
  modified_base 12: mod_base = cm
  modified_base 13: mod_base = OTHER; note = 2'-O-methyl adenosine
  modified_base 14: mod_base = gm
  modified_base 15: mod_base = um
  modified_base 16: mod_base = OTHER; note = 2'-O-methyl adenosine
  modified_base 17: mod_base = OTHER; note = 2'-O-methyl adenosine
  modified_base 18: mod_base = um
  modified_base 19: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
  modified_base 20: mod_base = OTHER; note = 2'-O-methyl adenosine
  modified_base 21: mod_base = OTHER; note = 2'-O-methyl adenosine
ttggacttgcacagtaataaa

SEQ ID NO: 323 - RNA, 18 nt, synthetic construct
  source 1..18: mol_type = other RNA
  modified_base 1: mod_base = OTHER; note = 2'-deoxy-2'-fluoro uridine
  modified_base 2: mod_base = OTHER; note = 2'-O-methyl adenosine
  modified_base 3: mod_base = cm
  modified_base 4: mod_base = um
  modified_base 5: mod_base = gm
  modified_base 6: mod_base = um
  modified_base 7: mod_base = gm
  modified_base 8: mod_base = cm
  modified_base 9: mod_base = OTHER; note = 2'-deoxy-2'-fluoro adenosine
  modified_base 10: mod_base = OTHER; note = 2'-O-methyl adenosine
  modified_base 11: mod_base = OTHER; note = 2'-deoxy-2'-fluoro guanosine
  modified_base 12: mod_base = um
  modified_base 13: mod_base = cm
  modified_base 14: mod_base = cm
  modified_base 15: mod_base = OTHER; note = 2'-O-methyl adenosine
  modified_base 16: mod_base = OTHER; note = 2'-O-methyl adenosine
  misc_feature 1: note = The Uf is attached to VPUmsUfsUmAm[N3T1-U-R]
  modified_base 16..18: mod_base = OTHER; note = phosphorothioate (PS) linkage
  modified_base 17: mod_base = OTHER; note = 2'-O-methyl adenosine
  modified_base 18: mod_base = um
tactgtgcaagtccaaat

SEQ ID NO: 324 - RNA, 21 nt, synthetic construct
  source 1..21: mol_type = other RNA
  modified_base 1: mod_base = um
  modified_base 2: mod_base = um
  modified_base 3: mod_base = gm
  modified_base 4: mod_base = gm
  modified_base 5: mod_base = OTHER; note = 2'-O-methyl adenosine
  modified_base 6: mod_base = cm
  modified_base 7: mod_base = OTHER; note = 2'-deoxy-2'-fluoro uridine
  modified_base 8: mod_base = um
  modified_base 9: mod_base = OTHER; note = 2'-deoxy-2'-fluoro guanosine
  modified_base 10: mod_base = OTHER; note = 2'-deoxy-2'-fluoro cytidine
  modified_base 11: mod_base = OTHER; note = 2'-deoxy-2'-fluoro adenosine
  modified_base 12: mod_base = cm
  modified_base 13: mod_base = OTHER; note = 2'-O-methyl adenosine
  modified_base 14: mod_base = gm
  modified_base 15: mod_base = um
  modified_base 16: mod_base = OTHER; note = 2'-O-methyl adenosine
  modified_base 17: mod_base = OTHER; note = 2'-O-methyl adenosine
  modified_base 18: mod_base = um
  modified_base 19: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
  modified_base 20: mod_base = OTHER; note = 2'-O-methyl adenosine
  modified_base 21: mod_base = OTHER; note = 2'-O-methyl adenosine
ttggacttgcacagtaataaa

SEQ ID NO: 325 - RNA, 16 nt, synthetic construct
  source 1..16: mol_type = other RNA
  modified_base 1: mod_base = cm
  modified_base 2: mod_base = um
  modified_base 3: mod_base = gm
  modified_base 4: mod_base = um
  modified_base 5: mod_base = gm
  modified_base 6: mod_base = cm
  modified_base 7: mod_base = OTHER; note = 2'-deoxy-2'-fluoro adenosine
  modified_base 8: mod_base = OTHER; note = 2'-O-methyl adenosine
  modified_base 9: mod_base = OTHER; note = 2'-deoxy-2'-fluoro guanosine
  modified_base 10: mod_base = um
  modified_base 11: mod_base = cm
  modified_base 12: mod_base = cm
  modified_base 13: mod_base = OTHER; note = 2'-O-methyl adenosine
  modified_base 14: mod_base = OTHER; note = 2'-O-methyl adenosine
  misc_feature 1: note = The Cm is attached to VPUmsUfsUmAmUmUf[N3T1-A-R]
  modified_base 14..16: mod_base = OTHER; note = phosphorothioate (PS) linkage
  modified_base 15: mod_base = OTHER; note = 2'-O-methyl adenosine
  modified_base 16: mod_base = um
ctgtgcaagtccaaat

SEQ ID NO: 326 - RNA, 21 nt, synthetic construct
  source 1..21: mol_type = other RNA
  modified_base 1: mod_base = um
  modified_base 2: mod_base = OTHER; note = 2'-O-methyl adenosine
  modified_base 3: mod_base = gm
  modified_base 4: mod_base = OTHER; note = 2'-O-methyl adenosine
  modified_base 5: mod_base = um
  modified_base 6: mod_base = OTHER; note = 2'-O-methyl adenosine
  modified_base 7: mod_base = OTHER; note = 2'-deoxy-2'-fluoro uridine
  modified_base 8: mod_base = gm
  modified_base 9: mod_base = OTHER; note = 2'-deoxy-2'-fluoro adenosine
  modified_base 10: mod_base = OTHER; note = 2'-deoxy-2'-fluoro guanosine
  modified_base 11: mod_base = OTHER; note = 2'-deoxy-2'-fluoro cytidine
  modified_base 12: mod_base = OTHER; note = 2'-O-methyl adenosine
  modified_base 13: mod_base = gm
  modified_base 14: mod_base = cm
  modified_base 15: mod_base = OTHER; note = 2'-O-methyl adenosine
  modified_base 16: mod_base = OTHER; note = 2'-O-methyl adenosine
  modified_base 17: mod_base = um
  modified_base 18: mod_base = gm
  modified_base 19: mod_base = OTHER; note = 2'-O-methyl adenosine
  modified_base 1..3: mod_base = OTHER; note = phosphorothioate (PS) linkage
  modified_base 19^20: mod_base = OTHER; note = phosphorothioate (PS) linkage
  misc_feature 21: note = The Am is attached through the phosphorothioate linkage to [N3T12-012-01]
  modified_base 20: mod_base = um
  modified_base 21: mod_base = OTHER; note = 2'-O-methyl adenosine
tagatatgagcagcaatgata

SEQ ID NO: 327 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = um
  modified_base 4: mod_base = cm
  modified_base 5: mod_base = OTHER; note = 2'-O-methyl adenosine
  modified_base 6: mod_base = OTHER; note = 2'-deoxy-2'-fluoro uridine
  modified_base 7: mod_base = um
  modified_base 8: mod_base = gm
  modified_base 9: mod_base = cm
  modified_base 10: mod_base = um
  modified_base 11: mod_base = gm
  modified_base 12: mod_base = cm
  modified_base 13: mod_base = um
  modified_base 14: mod_base = OTHER; note = 2'-deoxy-2'-fluoro cytidine
  modified_base 15: mod_base = OTHER; note = 2'-O-methyl adenosine
  modified_base 16: mod_base = OTHER; note = 2'-deoxy-2'-fluoro uridine
  modified_base 17: mod_base = OTHER; note = 2'-O-methyl adenosine
  modified_base 18: mod_base = um
  modified_base 19: mod_base = cm
  modified_base 20: mod_base = um
  modified_base 21: mod_base = OTHER; note = 2'-O-methyl adenosine
  modified_base 1..3: mod_base = OTHER; note = phosphorothioate (PS) linkage
  modified_base 21..23: mod_base = OTHER; note = phosphorothioate (PS) linkage
  modified_base 22: mod_base = OTHER; note = 2'-O-methyl adenosine
  modified_base 23: mod_base = OTHER; note = 2'-O-methyl adenosine
tatcattgctgctcatatctaaa

SEQ ID NO: 328 - RNA, 21 nt, synthetic construct
  source 1..21: mol_type = other RNA
  modified_base 1: mod_base = um
  modified_base 2: mod_base = OTHER; note = 2'-O-methyl adenosine
  modified_base 3: mod_base = gm
  modified_base 4: mod_base = OTHER; note = 2'-O-methyl adenosine
  modified_base 5: mod_base = um
  modified_base 6: mod_base = OTHER; note = 2'-O-methyl adenosine
  modified_base 7: mod_base = OTHER; note = 2'-deoxy-2'-fluoro uridine
  modified_base 8: mod_base = gm
  modified_base 9: mod_base = OTHER; note = 2'-deoxy-2'-fluoro adenosine
  modified_base 10: mod_base = OTHER; note = 2'-deoxy-2'-fluoro guanosine
  modified_base 11: mod_base = OTHER; note = 2'-deoxy-2'-fluoro cytidine
  modified_base 12: mod_base = OTHER; note = 2'-O-methyl adenosine
  modified_base 13: mod_base = gm
  modified_base 14: mod_base = cm
  modified_base 15: mod_base = OTHER; note = 2'-O-methyl adenosine
  modified_base 16: mod_base = OTHER; note = 2'-O-methyl adenosine
  modified_base 17: mod_base = um
  modified_base 18: mod_base = gm
  modified_base 19: mod_base = OTHER; note = 2'-O-methyl adenosine
  modified_base 1..3: mod_base = OTHER; note = phosphorothioate (PS) linkage
  modified_base 19^20: mod_base = OTHER; note = phosphorothioate (PS) linkage
  misc_feature 21: note = The Am is attached through the phosphorothioate linkage to [N3T12-012-01]
  modified_base 20: mod_base = um
  modified_base 21: mod_base = OTHER; note = 2'-O-methyl adenosine
tagatatgagcagcaatgata

SEQ ID NO: 329 - RNA, 18 nt, synthetic construct
  source 1..18: mol_type = other RNA
  modified_base 1: mod_base = OTHER; note = 2'-deoxy-2'-fluoro uridine
  modified_base 2: mod_base = um
  modified_base 3: mod_base = gm
  modified_base 4: mod_base = cm
  modified_base 5: mod_base = um
  modified_base 6: mod_base = gm
  modified_base 7: mod_base = cm
  modified_base 8: mod_base = um
  modified_base 9: mod_base = OTHER; note = 2'-deoxy-2'-fluoro cytidine
  modified_base 10: mod_base = OTHER; note = 2'-O-methyl adenosine
  modified_base 11: mod_base = OTHER; note = 2'-deoxy-2'-fluoro uridine
  modified_base 12: mod_base = OTHER; note = 2'-O-methyl adenosine
  modified_base 13: mod_base = um
  modified_base 14: mod_base = cm
  modified_base 15: mod_base = um
  modified_base 16: mod_base = OTHER; note = 2'-O-methyl adenosine
  misc_feature 1: note = The Uf is attached to VPUmsAfsUmCm[N3T1-A-R]
  modified_base 16..18: mod_base = OTHER; note = phosphorothioate (PS) linkage
  modified_base 17: mod_base = OTHER; note = 2'-O-methyl adenosine
  modified_base 18: mod_base = OTHER; note = 2'-O-methyl adenosine
ttgctgctcatatctaaa

SEQ ID NO: 330 - RNA, 21 nt, synthetic construct
  source 1..21: mol_type = other RNA
  modified_base 1: mod_base = um
  modified_base 2: mod_base = OTHER; note = 2'-O-methyl adenosine
  modified_base 3: mod_base = gm
  modified_base 4: mod_base = OTHER; note = 2'-O-methyl adenosine
  modified_base 5: mod_base = um
  modified_base 6: mod_base = OTHER; note = 2'-O-methyl adenosine
  modified_base 7: mod_base = OTHER; note = 2'-deoxy-2'-fluoro uridine
  modified_base 8: mod_base = gm
  modified_base 9: mod_base = OTHER; note = 2'-deoxy-2'-fluoro adenosine
  modified_base 10: mod_base = OTHER; note = 2'-deoxy-2'-fluoro guanosine
  modified_base 11: mod_base = OTHER; note = 2'-deoxy-2'-fluoro cytidine
  modified_base 12: mod_base = OTHER; note = 2'-O-methyl adenosine
  modified_base 13: mod_base = gm
  modified_base 14: mod_base = cm
  modified_base 15: mod_base = OTHER; note = 2'-O-methyl adenosine
  modified_base 16: mod_base = OTHER; note = 2'-O-methyl adenosine
  modified_base 17: mod_base = um
  modified_base 18: mod_base = gm
  modified_base 19: mod_base = OTHER; note = 2'-O-methyl adenosine
  modified_base 1..3: mod_base = OTHER; note = phosphorothioate (PS) linkage
  modified_base 19^20: mod_base = OTHER; note = phosphorothioate (PS) linkage
  misc_feature 21: note = The Am is attached through the phosphorothioate linkage to [N3T12-012-01]
  modified_base 20: mod_base = um
  modified_base 21: mod_base = OTHER; note = 2'-O-methyl adenosine
tagatatgagcagcaatgata

SEQ ID NO: 331 - RNA, 16 nt, synthetic construct
  source 1..16: mol_type = other RNA
  modified_base 1: mod_base = gm
  modified_base 2: mod_base = cm
  modified_base 3: mod_base = um
  modified_base 4: mod_base = gm
  modified_base 5: mod_base = cm
  modified_base 6: mod_base = um
  modified_base 7: mod_base = OTHER; note = 2'-deoxy-2'-fluoro cytidine
  modified_base 8: mod_base = OTHER; note = 2'-O-methyl adenosine
  modified_base 9: mod_base = OTHER; note = 2'-deoxy-2'-fluoro uridine
  modified_base 10: mod_base = OTHER; note = 2'-O-methyl adenosine
  modified_base 11: mod_base = um
  modified_base 12: mod_base = cm
  modified_base 13: mod_base = um
  modified_base 14: mod_base = OTHER; note = 2'-O-methyl adenosine
  misc_feature 1: note = The Gm is attached to VPUmsAfsUmCmAmUf[N3T1-U-R]
  modified_base 14..16: mod_base = OTHER; note = phosphorothioate (PS) linkage
  modified_base 15: mod_base = OTHER; note = 2'-O-methyl adenosine
  modified_base 16: mod_base = OTHER; note = 2'-O-methyl adenosine
gctgctcatatctaaa

SEQ ID NO: 332 - RNA, 15 nt, synthetic construct
  source 1..15: mol_type = other RNA
  misc_feature 1: note = The Uf is attached to CmsUmsUmGmUm(Uhd)
  modified_base 13..15: mod_base = OTHER; note = phosphorothioate (PS) linkage
  modified_base 1: mod_base = OTHER; note = 2'-deoxy-2'-fluoro uridine
  modified_base 2: mod_base = gm
  modified_base 3: mod_base = OTHER; note = 2'-deoxy-2'-fluoro uridine
  modified_base 4: mod_base = OTHER; note = 2'-deoxy-2'-fluoro adenosine
  modified_base 5: mod_base = OTHER; note = 2'-deoxy-2'-fluoro uridine
  modified_base 6: mod_base = um
  modified_base 7: mod_base = gm
  modified_base 8: mod_base = cm
  modified_base 9: mod_base = OTHER; note = 2'-O-methyl adenosine
  modified_base 10: mod_base = OTHER; note = 2'-O-methyl adenosine
  modified_base 11: mod_base = um
  modified_base 12: mod_base = um
  modified_base 14: mod_base = cm
  modified_base 13: mod_base = cm
  modified_base 15: mod_base = OTHER; note = 2'-O-methyl adenosine
tgtattgcaattcca

SEQ ID NO: 333 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro guanosine
  modified_base 3: mod_base = gm
  modified_base 4: mod_base = OTHER; note = 2'-O-methyl adenosine
  modified_base 5: mod_base = OTHER; note = 2'-O-methyl adenosine
  modified_base 6: mod_base = OTHER; note = 2'-deoxy-2'-fluoro uridine
  modified_base 7: mod_base = um
  modified_base 8: mod_base = gm
  modified_base 9: mod_base = cm
  modified_base 10: mod_base = OTHER; note = 2'-O-methyl adenosine
  modified_base 11: mod_base = OTHER; note = 2'-O-methyl adenosine
  modified_base 12: mod_base = um
  modified_base 13: mod_base = OTHER; note = 2'-O-methyl adenosine
  modified_base 14: mod_base = OTHER; note = 2'-deoxy-2'-fluoro cytidine
  modified_base 15: mod_base = OTHER; note = 2'-O-methyl adenosine
  modified_base 16: mod_base = OTHER; note = 2'-O-methyl adenosine
  modified_base 17: mod_base = OTHER; note = 2'-O-methyl adenosine
  modified_base 18: mod_base = cm
  modified_base 19: mod_base = OTHER; note = 2'-O-methyl adenosine
  modified_base 20: mod_base = OTHER; note = 2'-O-methyl adenosine
  modified_base 21: mod_base = gm
  modified_base 1..3: mod_base = OTHER; note = phosphorothioate (PS) linkage
  modified_base 21..23: mod_base = OTHER; note = phosphorothioate (PS) linkage
  modified_base 22: mod_base = um
  modified_base 23: mod_base = gm
tggaattgcaatacaaacaagtg

SEQ ID NO: 334 - RNA, 21 nt, synthetic construct
  source 1..21: mol_type = other RNA
  modified_base 1: mod_base = cm
  modified_base 2: mod_base = um
  modified_base 3: mod_base = um
  modified_base 4: mod_base = gm
  modified_base 5: mod_base = um
  modified_base 6: mod_base = um
  modified_base 7: mod_base = OTHER; note = 2'-deoxy-2'-fluoro uridine
  modified_base 8: mod_base = gm
  modified_base 9: mod_base = OTHER; note = 2'-deoxy-2'-fluoro uridine
  modified_base 10: mod_base = OTHER; note = 2'-deoxy-2'-fluoro adenosine
  modified_base 11: mod_base = OTHER; note = 2'-deoxy-2'-fluoro uridine
  modified_base 12: mod_base = um
  modified_base 13: mod_base = gm
  modified_base 14: mod_base = cm
  modified_base 15: mod_base = OTHER; note = 2'-O-methyl adenosine
  modified_base 16: mod_base = OTHER; note = 2'-O-methyl adenosine
  modified_base 17: mod_base = um
  modified_base 18: mod_base = um
  modified_base 19: mod_base = cm
  modified_base 20: mod_base = cm
  modified_base 21: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
cttgtttgtattgcaattcca

SEQ ID NO: 335 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro guanosine
  modified_base 3: mod_base = gm
  modified_base 4: mod_base = OTHER; note = 2'-O-methyl adenosine
  modified_base 5: mod_base = OTHER; note = 2'-O-methyl adenosine
  modified_base 6: mod_base = OTHER; note = 2'-deoxy-2'-fluoro uridine
  modified_base 7: mod_base = um
  modified_base 8: mod_base = gm
  modified_base 9: mod_base = cm
  modified_base 10: mod_base = OTHER; note = 2'-O-methyl adenosine
  modified_base 11: mod_base = OTHER; note = 2'-O-methyl adenosine
  modified_base 12: mod_base = um
  modified_base 13: mod_base = OTHER; note = 2'-O-methyl adenosine
  modified_base 14: mod_base = OTHER; note = 2'-deoxy-2'-fluoro cytidine
  modified_base 15: mod_base = OTHER; note = 2'-O-methyl adenosine
  modified_base 16: mod_base = OTHER; note = 2'-deoxy-2'-fluoro adenosine
  modified_base 17: mod_base = OTHER; note = 2'-O-methyl adenosine
  modified_base 18: mod_base = cm
  modified_base 19: mod_base = OTHER; note = 2'-O-methyl adenosine
  modified_base 20: mod_base = OTHER; note = 2'-O-methyl adenosine
  modified_base 21: mod_base = gm
  modified_base 1..3: mod_base = OTHER; note = phosphorothioate (PS) linkage
  modified_base 21..23: mod_base = OTHER; note = phosphorothioate (PS) linkage
  modified_base 22: mod_base = um
  modified_base 23: mod_base = gm
tggaattgcaatacaaacaagtg

SEQ ID NO: 336 - RNA, 21 nt, synthetic construct
  source 1..21: mol_type = other RNA
  modified_base 1: mod_base = um
  modified_base 2: mod_base = um
  modified_base 3: mod_base = gm
  modified_base 4: mod_base = gm
  modified_base 5: mod_base = OTHER; note = 2'-O-methyl adenosine
  modified_base 6: mod_base = cm
  modified_base 7: mod_base = OTHER; note = 2'-deoxy-2'-fluoro uridine
  modified_base 8: mod_base = um
  modified_base 9: mod_base = OTHER; note = 2'-deoxy-2'-fluoro guanosine
  modified_base 10: mod_base = OTHER; note = 2'-deoxy-2'-fluoro cytidine
  modified_base 11: mod_base = OTHER; note = 2'-deoxy-2'-fluoro adenosine
  modified_base 12: mod_base = cm
  modified_base 13: mod_base = OTHER; note = 2'-O-methyl adenosine
  modified_base 14: mod_base = gm
  modified_base 15: mod_base = um
  modified_base 16: mod_base = OTHER; note = 2'-O-methyl adenosine
  modified_base 17: mod_base = OTHER; note = 2'-O-methyl adenosine
  modified_base 18: mod_base = um
  modified_base 19: mod_base = OTHER; note = 2'-O-methyl adenosine
  modified_base 20: mod_base = OTHER; note = 2'-O-methyl adenosine
  modified_base 21: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
ttggacttgcacagtaataaa

SEQ ID NO: 337 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro uridine
  modified_base 3: mod_base = um
  modified_base 4: mod_base = OTHER; note = 2'-O-methyl adenosine
  modified_base 5: mod_base = um
  modified_base 6: mod_base = OTHER; note = 2'-deoxy-2'-fluoro uridine
  modified_base 7: mod_base = OTHER; note = 2'-O-methyl adenosine
  modified_base 8: mod_base = cm
  modified_base 9: mod_base = um
  modified_base 10: mod_base = gm
  modified_base 11: mod_base = um
  modified_base 12: mod_base = gm
  modified_base 13: mod_base = cm
  modified_base 14: mod_base = OTHER; note = 2'-deoxy-2'-fluoro adenosine
  modified_base 15: mod_base = OTHER; note = 2'-O-methyl adenosine
  modified_base 16: mod_base = OTHER; note = 2'-deoxy-2'-fluoro guanosine
  modified_base 17: mod_base = um
  modified_base 18: mod_base = cm
  modified_base 19: mod_base = cm
  modified_base 20: mod_base = OTHER; note = 2'-O-methyl adenosine
  modified_base 21: mod_base = OTHER; note = 2'-O-methyl adenosine
  modified_base 1..3: mod_base = OTHER; note = phosphorothioate (PS) linkage
  modified_base 21..23: mod_base = OTHER; note = phosphorothioate (PS) linkage
  modified_base 22: mod_base = OTHER; note = 2'-O-methyl adenosine
  modified_base 23: mod_base = um
tttattactgtgcaagtccaaat

SEQ ID NO: 338 - RNA, 21 nt, synthetic construct
  source 1..21: mol_type = other RNA
  modified_base 1: mod_base = cm
  modified_base 2: mod_base = OTHER; note = 2'-O-methyl adenosine
  modified_base 3: mod_base = gm
  modified_base 4: mod_base = gm
  modified_base 5: mod_base = um
  modified_base 6: mod_base = cm
  modified_base 7: mod_base = OTHER; note = 2'-deoxy-2'-fluoro guanosine
  modified_base 8: mod_base = um
  modified_base 9: mod_base = OTHER; note = 2'-deoxy-2'-fluoro guanosine
  modified_base 10: mod_base = OTHER; note = 2'-deoxy-2'-fluoro adenosine
  modified_base 11: mod_base = OTHER; note = 2'-deoxy-2'-fluoro guanosine
  modified_base 12: mod_base = gm
  modified_base 13: mod_base = OTHER; note = 2'-O-methyl adenosine
  modified_base 14: mod_base = OTHER; note = 2'-O-methyl adenosine
  modified_base 15: mod_base = um
  modified_base 16: mod_base = um
  modified_base 17: mod_base = cm
  modified_base 18: mod_base = um
  modified_base 19: mod_base = OTHER; note = 2'-O-methyl adenosine
  modified_base 20: mod_base = um
  modified_base 21: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
caggtcgtgaggaattctata

SEQ ID NO: 339 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = um
  modified_base 4: mod_base = OTHER; note = 2'-O-methyl adenosine
  modified_base 5: mod_base = gm
  modified_base 6: mod_base = OTHER; note = 2'-deoxy-2'-fluoro adenosine
  modified_base 7: mod_base = OTHER; note = 2'-O-methyl adenosine
  modified_base 8: mod_base = um
  modified_base 9: mod_base = um
  modified_base 10: mod_base = cm
  modified_base 11: mod_base = cm
  modified_base 12: mod_base = um
  modified_base 13: mod_base = cm
  modified_base 14: mod_base = OTHER; note = 2'-deoxy-2'-fluoro adenosine
  modified_base 15: mod_base = cm
  modified_base 16: mod_base = OTHER; note = 2'-deoxy-2'-fluoro guanosine
  modified_base 17: mod_base = OTHER; note = 2'-O-methyl adenosine
  modified_base 18: mod_base = cm
  modified_base 19: mod_base = cm
  modified_base 20: mod_base = um
  modified_base 21: mod_base = gm
  modified_base 1..3: mod_base = OTHER; note = phosphorothioate (PS) linkage
  modified_base 21..23: mod_base = OTHER; note = phosphorothioate (PS) linkage
  modified_base 22: mod_base = cm
  modified_base 23: mod_base = OTHER; note = 2'-O-methyl adenosine
tatagaattcctcacgacctgca

SEQ ID NO: 340 - RNA, 21 nt, synthetic construct
  source 1..21: mol_type = other RNA
  modified_base 1: mod_base = um
  modified_base 2: mod_base = OTHER; note = 2'-O-methyl adenosine
  modified_base 3: mod_base = gm
  modified_base 4: mod_base = OTHER; note = 2'-O-methyl adenosine
  modified_base 5: mod_base = um
  modified_base 6: mod_base = OTHER; note = 2'-O-methyl adenosine
  modified_base 7: mod_base = OTHER; note = 2'-deoxy-2'-fluoro uridine
  modified_base 8: mod_base = gm
  modified_base 9: mod_base = OTHER; note = 2'-deoxy-2'-fluoro adenosine
  modified_base 10: mod_base = OTHER; note = 2'-deoxy-2'-fluoro guanosine
  modified_base 11: mod_base = OTHER; note = 2'-deoxy-2'-fluoro cytidine
  modified_base 12: mod_base = OTHER; note = 2'-O-methyl adenosine
  modified_base 13: mod_base = gm
  modified_base 14: mod_base = cm
  modified_base 15: mod_base = OTHER; note = 2'-O-methyl adenosine
  modified_base 16: mod_base = OTHER; note = 2'-O-methyl adenosine
  modified_base 17: mod_base = um
  modified_base 18: mod_base = gm
  modified_base 19: mod_base = OTHER; note = 2'-O-methyl adenosine
  modified_base 20: mod_base = um
  modified_base 21: mod_base = OTHER; note = 2'-O-methyl adenosine
  modified_base 1..3: mod_base = OTHER; note = phosphorothioate (PS) linkage
  modified_base 20^21: mod_base = OTHER; note = phosphorothioate (PS) linkage
  misc_feature 21: note = The Am is attached through the phosphorothioate linkage to [N3T12-012-01]
tagatatgagcagcaatgata

SEQ ID NO: 341 - RNA, 23 nt, synthetic construct
  source 1..23: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = um
  modified_base 4: mod_base = cm
  modified_base 5: mod_base = OTHER; note = 2'-O-methyl adenosine
  modified_base 6: mod_base = OTHER; note = 2'-deoxy-2'-fluoro uridine
  modified_base 7: mod_base = um
  modified_base 8: mod_base = gm
  modified_base 9: mod_base = cm
  modified_base 10: mod_base = um
  modified_base 11: mod_base = gm
  modified_base 12: mod_base = cm
  modified_base 13: mod_base = um
  modified_base 14: mod_base = OTHER; note = 2'-deoxy-2'-fluoro cytidine
  modified_base 15: mod_base = OTHER; note = 2'-O-methyl adenosine
  modified_base 16: mod_base = OTHER; note = 2'-deoxy-2'-fluoro uridine
  modified_base 17: mod_base = OTHER; note = 2'-O-methyl adenosine
  modified_base 18: mod_base = um
  modified_base 19: mod_base = cm
  modified_base 20: mod_base = um
  modified_base 21: mod_base = OTHER; note = 2'-O-methyl adenosine
  modified_base 1..3: mod_base = OTHER; note = phosphorothioate (PS) linkage
  modified_base 21..23: mod_base = OTHER; note = phosphorothioate (PS) linkage
  modified_base 22: mod_base = OTHER; note = 2'-O-methyl adenosine
  modified_base 23: mod_base = OTHER; note = 2'-O-methyl adenosine
tatcattgctgctcatatctaaa

SEQ ID NO: 342 - RNA, 19 nt, synthetic construct
  source 1..19: mol_type = other RNA
tgacaagaatcctcacaat

SEQ ID NO: 343 - RNA, 21 nt, synthetic construct
  source 1..21: mol_type = other RNA
attgtgaggattcttgtcaac

SEQ ID NO: 344 - RNA, 19 nt, synthetic construct
  source 1..19: mol_type = other RNA
tgacaagaatcctcacaat

SEQ ID NO: 345 - RNA, 14 nt, synthetic construct
  source 1..14: mol_type = other RNA
  misc_feature 1: note = The G is attached to AUUGUG[GNA-A]
ggattcttgtcaac

SEQ ID NO: 346 - RNA, 19 nt, synthetic construct
  source 1..19: mol_type = other RNA
tgacaagaatcctcacaat

SEQ ID NO: 347 - RNA, 14 nt, synthetic construct
  source 1..14: mol_type = other RNA
  misc_feature 1: note = The G is attached to AUUGUG[N3T1-A-S]
ggattcttgtcaac

SEQ ID NO: 348 - RNA, 19 nt, synthetic construct
  source 1..19: mol_type = other RNA
tgacaagaatcctcacaat

SEQ ID NO: 349 - RNA, 14 nt, synthetic construct
  source 1..14: mol_type = other RNA
  misc_feature 1: note = The G is attached to AUUGUG[N3T1-A-R]
ggattcttgtcaac

SEQ ID NO: 350 - RNA, 19 nt, synthetic construct
  source 1..19: mol_type = other RNA
tgacaagaatcctcacaat

SEQ ID NO: 351 - RNA, 16 nt, synthetic construct
  source 1..16: mol_type = other RNA
  misc_feature 1: note = The G is attached to AUUG[GNA-U]
gaggattcttgtcaac

SEQ ID NO: 352 - RNA, 19 nt, synthetic construct
  source 1..19: mol_type = other RNA
tgacaagaatcctcacaat

SEQ ID NO: 353 - RNA, 16 nt, synthetic construct
  source 1..16: mol_type = other RNA
  misc_feature 1: note = The G is attached to AUUG[N3T1-U-S]
gaggattcttgtcaac

SEQ ID NO: 354 - RNA, 19 nt, synthetic construct
  source 1..19: mol_type = other RNA
tgacaagaatcctcacaat

SEQ ID NO: 355 - RNA, 16 nt, synthetic construct
  source 1..16: mol_type = other RNA
  misc_feature 1: note = The G is attached to AUUG[N3T1-U-R]
gaggattcttgtcaac

SEQ ID NO: 356 - RNA, 19 nt, synthetic construct
  source 1..19: mol_type = other RNA
tgaatgcagaccaaagaaa

SEQ ID NO: 357 - RNA, 21 nt, synthetic construct
  source 1..21: mol_type = other RNA
tttctttggtctgcattcaca

SEQ ID NO: 358 - RNA, 19 nt, synthetic construct
  source 1..19: mol_type = other RNA
tgaatgcagaccaaagaaa

SEQ ID NO: 359 - RNA, 14 nt, synthetic construct
  source 1..14: mol_type = other RNA
  misc_feature 1: note = The G is attached to UUUCUU[GNA-U]
ggtctgcattcaca

SEQ ID NO: 360 - RNA, 19 nt, synthetic construct
  source 1..19: mol_type = other RNA
tgaatgcagaccaaagaaa

SEQ ID NO: 361 - RNA, 14 nt, synthetic construct
  source 1..14: mol_type = other RNA
  misc_feature 1: note = The G is attached to UUUCUU[N3T1-U-S]
ggtctgcattcaca

SEQ ID NO: 362 - RNA, 19 nt, synthetic construct
  source 1..19: mol_type = other RNA
tgaatgcagaccaaagaaa

SEQ ID NO: 363 - RNA, 14 nt, synthetic construct
  source 1..14: mol_type = other RNA
  misc_feature 1: note = The G is attached to UUUCUU[N3T1-U-R]
ggtctgcattcaca

SEQ ID NO: 364 - RNA, 19 nt, synthetic construct
  source 1..19: mol_type = other RNA
tgaatgcagaccaaagaaa

SEQ ID NO: 365 - RNA, 16 nt, synthetic construct
  source 1..16: mol_type = other RNA
  misc_feature 1: note = The G is attached to UUUC[GNA-U]
ttggtctgcattcaca

SEQ ID NO: 366 - RNA, 19 nt, synthetic construct
  source 1..19: mol_type = other RNA
tgaatgcagaccaaagaaa

SEQ ID NO: 367 - RNA, 16 nt, synthetic construct
  source 1..16: mol_type = other RNA
  misc_feature 1: note = The G is attached to UUUC[N3T1-U-S]
ttggtctgcattcaca

SEQ ID NO: 368 - RNA, 19 nt, synthetic construct
  source 1..19: mol_type = other RNA
tgaatgcagaccaaagaaa

SEQ ID NO: 369 - RNA, 16 nt, synthetic construct
  source 1..16: mol_type = other RNA
  misc_feature 1: note = The G is attached to UUUC[N3T1-U-R]
ttggtctgcattcaca

SEQ ID NO: 370 - RNA, 19 nt, synthetic construct
  source 1..19: mol_type = other RNA
  modified_base 1: mod_base = gm
  modified_base 2: mod_base = um
  modified_base 3: mod_base = OTHER; note = 2'-O-methyl adenosine
  modified_base 4: mod_base = OTHER; note = 2'-O-methyl adenosine
  modified_base 5: mod_base = OTHER; note = 2'-deoxy-2'-fluoro adenosine
  modified_base 6: mod_base = um
  modified_base 7: mod_base = OTHER; note = 2'-deoxy-2'-fluoro adenosine
  modified_base 8: mod_base = OTHER; note = 2'-deoxy-2'-fluoro adenosine
  modified_base 9: mod_base = OTHER; note = 2'-deoxy-2'-fluoro cytidine
  modified_base 10: mod_base = um
  modified_base 11: mod_base = gm
  modified_base 12: mod_base = gm
  modified_base 13: mod_base = OTHER; note = 2'-O-methyl adenosine
  modified_base 14: mod_base = OTHER; note = 2'-O-methyl adenosine
  modified_base 15: mod_base = OTHER; note = 2'-O-methyl adenosine
  modified_base 16: mod_base = OTHER; note = 2'-O-methyl adenosine
  modified_base 17: mod_base = cm
  modified_base 18: mod_base = OTHER; note = 2'-O-methyl adenosine
  modified_base 19: mod_base = OTHER; note = 2'-O-methyl adenosine
  modified_base 1..3: mod_base = OTHER; note = phosphorothioate (PS) linkage
  modified_base 18^19: mod_base = OTHER; note = phosphorothioate (PS) linkage
  misc_feature 19: note = The Am is attached through the phosphorothioate linkage to [C5Cholesterol]
gtaaataactggaaaacaa

SEQ ID NO: 371 - RNA, 21 nt, synthetic construct
  source 1..21: mol_type = other RNA
  modified_base 1: mod_base = OTHER; note = vinyl phosphonate 2'-O-methyl uridine
  modified_base 2: mod_base = OTHER; note = 2'-deoxy-2'-fluoro uridine
  modified_base 3: mod_base = gm
  modified_base 4: mod_base = um
  modified_base 5: mod_base = um
  modified_base 6: mod_base = OTHER; note = 2'-deoxy-2'-fluoro uridine
  modified_base 7: mod_base = um
  modified_base 8: mod_base = OTHER; note = 2'-deoxy-2'-fluoro cytidine
  modified_base 9: mod_base = OTHER; note = 2'-deoxy-2'-fluoro cytidine
  modified_base 10: mod_base = OTHER; note = 2'-O-methyl adenosine
  modified_base 11: mod_base = gm
  modified_base 12: mod_base = um
  modified_base 13: mod_base = um
  modified_base 14: mod_base = OTHER; note = 2'-deoxy-2'-fluoro adenosine
  modified_base 15: mod_base = um
  modified_base 16: mod_base = OTHER; note = 2'-deoxy-2'-fluoro uridine
  modified_base 17: mod_base = um
  modified_base 18: mod_base = OTHER; note = 2'-O-methyl adenosine
  modified_base 19: mod_base = cm
  modified_base 20: mod_base = um
  modified_base 21: mod_base = gm
  modified_base 1..3: mod_base = OTHER; note = phosphorothioate (PS) linkage
  modified_base 19..21: mod_base = OTHER; note = phosphorothioate (PS) linkage
ttgttttccagttatttactg

SEQ ID NO: 372 - RNA, 19 nt, synthetic construct
  source 1..19: mol_type = other RNA
  modified_base 1: mod_base = gm
  modified_base 2: mod_base = um
  modified_base 3: mod_base = OTHER; note = 2'-O-methyl adenosine
  modified_base 4: mod_base = OTHER; note = 2'-O-methyl adenosine
  modified_base 5: mod_base = OTHER; note = 2'-deoxy-2'-fluoro adenosine
  modified_base 6: mod_base = um
  modified_base 7: mod_base = OTHER; note = 2'-deoxy-2'-fluoro adenosine
  modified_base 8: mod_base = OTHER; note = 2'-deoxy-2'-fluoro adenosine
  modified_base 9: mod_base = OTHER; note = 2'-deoxy-2'-fluoro cytidine
  modified_base 10: mod_base = um
  modified_base 11: mod_base = gm
  modified_base 12: mod_base = gm
  modified_base 13: mod_base = OTHER; note = 2'-O-methyl adenosine
  modified_base 14: mod_base = OTHER; note = 2'-O-methyl adenosine
  modified_base 15: mod_base = OTHER; note = 2'-O-methyl adenosine
  modified_base 16: mod_base = OTHER; note = 2'-O-methyl adenosine
  modified_base 17: mod_base = cm
  modified_base 18: mod_base = OTHER; note = 2'-O-methyl adenosine
  modified_base 19: mod_base = OTHER; note = 2'-O-methyl adenosine
  modified_base 1..3: mod_base = OTHER; note = phosphorothioate (PS) linkage
  modified_base 18^19: mod_base = OTHER; note = phosphorothioate (PS) linkage
  misc_feature 19: note = The Am is attached through the phosphorothioate linkage to [C5Cholesterol]
gtaaataactggaaaacaa

SEQ ID NO: 373 - RNA, 16 nt, synthetic construct
  source 1..16: mol_type = other RNA
  modified_base 1: mod_base = OTHER; note = 2'-deoxy-2'-fluoro uridine
  modified_base 2: mod_base = um
  modified_base 3: mod_base = OTHER; note = 2'-deoxy-2'-fluoro cytidine
  modified_base 4: mod_base = OTHER; note = 2'-deoxy-2'-fluoro cytidine
  modified_base 5: mod_base = OTHER; note = 2'-O-methyl adenosine
  modified_base 6: mod_base = gm
  modified_base 7: mod_base = um
  modified_base 8: mod_base = um
  modified_base 9: mod_base = OTHER; note = 2'-deoxy-2'-fluoro adenosine
  modified_base 10: mod_base = um
  modified_base 11: mod_base = OTHER; note = 2'-deoxy-2'-fluoro uridine
  modified_base 12: mod_base = um
  modified_base 13: mod_base = OTHER; note = 2'-O-methyl adenosine
  modified_base 14: mod_base = cm
  modified_base 15: mod_base = um
  modified_base 16: mod_base = gm
  misc_feature 1: note = The Uf is attached to VPUmsUfsGmUm[N3T1-U-R]
  modified_base 14..16: mod_base = OTHER; note = phosphorothioate (PS) linkage
ttccagttatttactg

SEQ ID NO: 374 - RNA, 19 nt, synthetic construct
  source 1..19: mol_type = other RNA
  modified_base 1: mod_base = gm
  modified_base 2: mod_base = um
  modified_base 3: mod_base = OTHER; note = 2'-O-methyl adenosine
  modified_base 4: mod_base = OTHER; note = 2'-O-methyl adenosine
  modified_base 5: mod_base = OTHER; note = 2'-deoxy-2'-fluoro adenosine
  modified_base 6: mod_base = um
  modified_base 7: mod_base = OTHER; note = 2'-deoxy-2'-fluoro adenosine
  modified_base 8: mod_base = OTHER; note = 2'-deoxy-2'-fluoro adenosine
  modified_base 9: mod_base = OTHER; note = 2'-deoxy-2'-fluoro cytidine
  modified_base 10: mod_base = um
  modified_base 11: mod_base = gm
  modified_base 12: mod_base = gm
  modified_base 13: mod_base = OTHER; note = 2'-O-methyl adenosine
  modified_base 14: mod_base = OTHER; note = 2'-O-methyl adenosine
  modified_base 15: mod_base = OTHER; note = 2'-O-methyl adenosine
  modified_base 16: mod_base = OTHER; note = 2'-O-methyl adenosine
  modified_base 17: mod_base = cm
  modified_base 18: mod_base = OTHER; note = 2'-O-methyl adenosine
  modified_base 19: mod_base = OTHER; note = 2'-O-methyl adenosine
  modified_base 1..3: mod_base = OTHER; note = phosphorothioate (PS) linkage
  modified_base 18^19: mod_base = OTHER; note = phosphorothioate (PS) linkage
  misc_feature 19: note = The Am is attached through the phosphorothioate linkage to [C5Cholesterol]
gtaaataactggaaaacaa

SEQ ID NO: 375 - RNA, 14 nt, synthetic construct
  source 1..14: mol_type = other RNA
  modified_base 1: mod_base = OTHER; note = 2'-deoxy-2'-fluoro cytidine
  modified_base 2: mod_base = OTHER; note = 2'-deoxy-2'-fluoro cytidine
  modified_base 3: mod_base = OTHER; note = 2'-O-methyl adenosine
  modified_base 4: mod_base = gm
  modified_base 5: mod_base = um
  modified_base 6: mod_base = um
  modified_base 7: mod_base = OTHER; note = 2'-deoxy-2'-fluoro adenosine
  modified_base 8: mod_base = um
  modified_base 9: mod_base = OTHER; note = 2'-deoxy-2'-fluoro uridine
  modified_base 10: mod_base = um
  modified_base 11: mod_base = OTHER; note = 2'-O-methyl adenosine
  modified_base 12: mod_base = cm
  modified_base 13: mod_base = um
  modified_base 14: mod_base = gm
  misc_feature 1: note = The Cf is attached to VPUmsUfsGmUmUmUf[N3T1-U-R]
  modified_base 12..14: mod_base = OTHER; note = phosphorothioate (PS) linkage
ccagttatttactg

SEQ ID NO: 376 - RNA, 19 nt, synthetic construct
  source 1..19: mol_type = other RNA
  modified_base 1: mod_base = gm
  modified_base 2: mod_base = um
  modified_base 3: mod_base = OTHER; note = 2'-O-methyl adenosine
  modified_base 4: mod_base = OTHER; note = 2'-O-methyl adenosine
  modified_base 5: mod_base = OTHER; note = 2'-deoxy-2'-fluoro adenosine
  modified_base 6: mod_base = um
  modified_base 7: mod_base = OTHER; note = 2'-deoxy-2'-fluoro adenosine
  modified_base 8: mod_base = OTHER; note = 2'-deoxy-2'-fluoro adenosine
  modified_base 9: mod_base = OTHER; note = 2'-deoxy-2'-fluoro cytidine
  modified_base 10: mod_base = um
  modified_base 11: mod_base = gm
  modified_base 12: mod_base = gm
  modified_base 13: mod_base = OTHER; note = 2'-O-methyl adenosine
  modified_base 14: mod_base = OTHER; note = 2'-O-methyl adenosine
  modified_base 15: mod_base = OTHER; note = 2'-O-methyl adenosine
  modified_base 16: mod_base = OTHER; note = 2'-O-methyl adenosine
  modified_base 17: mod_base = cm
  modified_base 18: mod_base = OTHER; note = 2'-O-methyl adenosine
  modified_base 19: mod_base = OTHER; note = 2'-O-methyl adenosine
  modified_base 1..3: mod_base = OTHER; note = phosphorothioate (PS) linkage
  modified_base 18^19: mod_base = OTHER; note = phosphorothioate (PS) linkage
  misc_feature 19: note = The Am is attached through the phosphorothioate linkage to [C5Cholesterol]
gtaaataactggaaaacaa

SEQ ID NO: 377 - RNA, 14 nt, synthetic construct
  source 1..14: mol_type = other RNA
  modified_base 1: mod_base = OTHER; note = 2'-deoxy-2'-fluoro cytidine
  modified_base 2: mod_base = OTHER; note = 2'-deoxy-2'-fluoro cytidine
  modified_base 3: mod_base = OTHER; note = 2'-O-methyl adenosine
  modified_base 4: mod_base = gm
  modified_base 5: mod_base = um
  modified_base 6: mod_base = um
  modified_base 7: mod_base = OTHER; note = 2'-deoxy-2'-fluoro adenosine
  modified_base 8: mod_base = um
  modified_base 9: mod_base = OTHER; note = 2'-deoxy-2'-fluoro uridine
  modified_base 10: mod_base = um
  modified_base 11: mod_base = OTHER; note = 2'-O-methyl adenosine
  modified_base 12: mod_base = cm
  modified_base 13: mod_base = um
  modified_base 14: mod_base = gm
  misc_feature 1: note = The Cf is attached to VPUmsUfsGmUmUmUf[GNA-U]
  modified_base 12..14: mod_base = OTHER; note = phosphorothioate (PS) linkage
ccagttatttactg

SEQ ID NO: 378 - RNA, 19 nt, synthetic construct
  source 1..19: mol_type = other RNA
  modified_base 1: mod_base = OTHER; note = 2'-O-methyl adenosine
  modified_base 2: mod_base = um
  modified_base 3: mod_base = gm
  modified_base 4: mod_base = um
  modified_base 5: mod_base = OTHER; note = 2'-deoxy-2'-fluoro guanosine
  modified_base 6: mod_base = gm
  modified_base 7: mod_base = OTHER; note = 2'-deoxy-2'-fluoro uridine
  modified_base 8: mod_base = OTHER; note = 2'-deoxy-2'-fluoro cytidine
  modified_base 9: mod_base = OTHER; note = 2'-deoxy-2'-fluoro cytidine
  modified_base 10: mod_base = um
  modified_base 11: mod_base = um
  modified_base 12: mod_base = cm
  modified_base 13: mod_base = cm
  modified_base 14: mod_base = OTHER; note = 2'-O-methyl adenosine
  modified_base 15: mod_base = OTHER; note = 2'-O-methyl adenosine
  modified_base 16: mod_base = cm
  modified_base 17: mod_base = um
  modified_base 18: mod_base = um
  modified_base 19: mod_base = OTHER; note = 2'-O-methyl adenosine
  modified_base 1..3: mod_base = OTHER; note = phosphorothioate (PS) linkage
  modified_base 18^19: mod_base = OTHER; note = phosphorothioate (PS) linkage
  misc_feature 19: note = The Am is attached through the phosphorothioate linkage to [C5Cholesterol]
atgtggtccttccaactta

SEQ ID NO: 379 - RNA, 21 nt, synthetic construct
  source 1..21: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = OTHER; note = 2'-O-methyl adenosine
  modified_base 4: mod_base = gm
  modified_base 5: mod_base = um
  modified_base 6: mod_base = OTHER; note = 2'-deoxy-2'-fluoro uridine
  modified_base 7: mod_base = gm
  modified_base 8: mod_base = OTHER; note = 2'-deoxy-2'-fluoro guanosine
  modified_base 9: mod_base = OTHER; note = 2'-deoxy-2'-fluoro adenosine
  modified_base 10: mod_base = OTHER; note = 2'-O-methyl adenosine
  modified_base 11: mod_base = gm
  modified_base 12: mod_base = gm
  modified_base 13: mod_base = OTHER; note = 2'-O-methyl adenosine
  modified_base 14: mod_base = OTHER; note = 2'-deoxy-2'-fluoro cytidine
  modified_base 15: mod_base = cm
  modified_base 16: mod_base = OTHER; note = 2'-deoxy-2'-fluoro adenosine
  modified_base 17: mod_base = cm
  modified_base 18: mod_base = OTHER; note = 2'-O-methyl adenosine
  modified_base 19: mod_base = um
  modified_base 20: mod_base = gm
  modified_base 21: mod_base = cm
  modified_base 1..3: mod_base = OTHER; note = phosphorothioate (PS) linkage
  modified_base 19..21: mod_base = OTHER; note = phosphorothioate (PS) linkage
taagttggaaggaccacatgc

SEQ ID NO: 380 - RNA, 19 nt, synthetic construct
  source 1..19: mol_type = other RNA
  modified_base 1: mod_base = OTHER; note = 2'-O-methyl adenosine
  modified_base 2: mod_base = um
  modified_base 3: mod_base = gm
  modified_base 4: mod_base = um
  modified_base 5: mod_base = OTHER; note = 2'-deoxy-2'-fluoro guanosine
  modified_base 6: mod_base = gm
  modified_base 7: mod_base = OTHER; note = 2'-deoxy-2'-fluoro uridine
  modified_base 8: mod_base = OTHER; note = 2'-deoxy-2'-fluoro cytidine
  modified_base 9: mod_base = OTHER; note = 2'-deoxy-2'-fluoro cytidine
  modified_base 10: mod_base = um
  modified_base 11: mod_base = um
  modified_base 12: mod_base = cm
  modified_base 13: mod_base = cm
  modified_base 14: mod_base = OTHER; note = 2'-O-methyl adenosine
  modified_base 15: mod_base = OTHER; note = 2'-O-methyl adenosine
  modified_base 16: mod_base = cm
  modified_base 17: mod_base = um
  modified_base 18: mod_base = um
  modified_base 19: mod_base = OTHER; note = 2'-O-methyl adenosine
  modified_base 1..3: mod_base = OTHER; note = phosphorothioate (PS) linkage
  modified_base 18^19: mod_base = OTHER; note = phosphorothioate (PS) linkage
  misc_feature 19: note = The Am is attached through the phosphorothioate linkage to [C5Cholesterol]
atgtggtccttccaactta

SEQ ID NO: 381 - RNA, 16 nt, synthetic construct
  source 1..16: mol_type = other RNA
  modified_base 1: mod_base = OTHER; note = 2'-deoxy-2'-fluoro uridine
  modified_base 2: mod_base = gm
  modified_base 3: mod_base = OTHER; note = 2'-deoxy-2'-fluoro guanosine
  modified_base 4: mod_base = OTHER; note = 2'-deoxy-2'-fluoro adenosine
  modified_base 5: mod_base = OTHER; note = 2'-O-methyl adenosine
  modified_base 6: mod_base = gm
  modified_base 7: mod_base = gm
  modified_base 8: mod_base = OTHER; note = 2'-O-methyl adenosine
  modified_base 9: mod_base = OTHER; note = 2'-deoxy-2'-fluoro cytidine
  modified_base 10: mod_base = cm
  modified_base 11: mod_base = OTHER; note = 2'-deoxy-2'-fluoro adenosine
  modified_base 12: mod_base = cm
  modified_base 13: mod_base = OTHER; note = 2'-O-methyl adenosine
  modified_base 14: mod_base = um
  modified_base 15: mod_base = gm
  modified_base 16: mod_base = cm
  misc_feature 1: note = The Uf is attached to VPUmsAfsAmGm[N3T1-U-R]
  modified_base 14..16: mod_base = OTHER; note = phosphorothioate (PS) linkage
tggaaggaccacatgc

SEQ ID NO: 382 - RNA, 19 nt, synthetic construct
  source 1..19: mol_type = other RNA
  modified_base 1: mod_base = OTHER; note = 2'-O-methyl adenosine
  modified_base 2: mod_base = um
  modified_base 3: mod_base = gm
  modified_base 4: mod_base = um
  modified_base 5: mod_base = OTHER; note = 2'-deoxy-2'-fluoro guanosine
  modified_base 6: mod_base = gm
  modified_base 7: mod_base = OTHER; note = 2'-deoxy-2'-fluoro uridine
  modified_base 8: mod_base = OTHER; note = 2'-deoxy-2'-fluoro cytidine
  modified_base 9: mod_base = OTHER; note = 2'-deoxy-2'-fluoro cytidine
  modified_base 10: mod_base = um
  modified_base 11: mod_base = um
  modified_base 12: mod_base = cm
  modified_base 13: mod_base = cm
  modified_base 14: mod_base = OTHER; note = 2'-O-methyl adenosine
  modified_base 15: mod_base = OTHER; note = 2'-O-methyl adenosine
  modified_base 16: mod_base = cm
  modified_base 17: mod_base = um
  modified_base 18: mod_base = um
  modified_base 19: mod_base = OTHER; note = 2'-O-methyl adenosine
  modified_base 1..3: mod_base = OTHER; note = phosphorothioate (PS) linkage
  modified_base 18^19: mod_base = OTHER; note = phosphorothioate (PS) linkage
  misc_feature 19: note = The Am is attached through the phosphorothioate linkage to [C5Cholesterol]
atgtggtccttccaactta

SEQ ID NO: 383 - RNA, 14 nt, synthetic construct
  source 1..14: mol_type = other RNA
  modified_base 1: mod_base = OTHER; note = 2'-deoxy-2'-fluoro guanosine
  modified_base 2: mod_base = OTHER; note = 2'-deoxy-2'-fluoro adenosine
  modified_base 3: mod_base = OTHER; note = 2'-O-methyl adenosine
  modified_base 4: mod_base = gm
  modified_base 5: mod_base = gm
  modified_base 6: mod_base = OTHER; note = 2'-O-methyl adenosine
  modified_base 7: mod_base = OTHER; note = 2'-deoxy-2'-fluoro cytidine
  modified_base 8: mod_base = cm
  modified_base 9: mod_base = OTHER; note = 2'-deoxy-2'-fluoro adenosine
  modified_base 10: mod_base = cm
  modified_base 11: mod_base = OTHER; note = 2'-O-methyl adenosine
  modified_base 12: mod_base = um
  modified_base 13: mod_base = gm
  modified_base 14: mod_base = cm
  misc_feature 1: note = The Gf is attached to VPUmsAfsAmGmUmUf[N3T1-G-R]
  modified_base 12..14: mod_base = OTHER; note = phosphorothioate (PS) linkage
gaaggaccacatgc

SEQ ID NO: 384 - RNA, 19 nt, synthetic construct
  source 1..19: mol_type = other RNA
  modified_base 1: mod_base = OTHER; note = 2'-O-methyl adenosine
  modified_base 2: mod_base = um
  modified_base 3: mod_base = gm
  modified_base 4: mod_base = um
  modified_base 5: mod_base = OTHER; note = 2'-deoxy-2'-fluoro guanosine
  modified_base 6: mod_base = gm
  modified_base 7: mod_base = OTHER; note = 2'-deoxy-2'-fluoro uridine
  modified_base 8: mod_base = OTHER; note = 2'-deoxy-2'-fluoro cytidine
  modified_base 9: mod_base = OTHER; note = 2'-deoxy-2'-fluoro cytidine
  modified_base 10: mod_base = um
  modified_base 11: mod_base = um
  modified_base 12: mod_base = cm
  modified_base 13: mod_base = cm
  modified_base 14: mod_base = OTHER; note = 2'-O-methyl adenosine
  modified_base 15: mod_base = OTHER; note = 2'-O-methyl adenosine
  modified_base 16: mod_base = cm
  modified_base 17: mod_base = um
  modified_base 18: mod_base = um
  modified_base 19: mod_base = OTHER; note = 2'-O-methyl adenosine
  modified_base 1..3: mod_base = OTHER; note = phosphorothioate (PS) linkage
  modified_base 18^19: mod_base = OTHER; note = phosphorothioate (PS) linkage
  misc_feature 19: note = The Am is attached through the phosphorothioate linkage to [C5Cholesterol]
atgtggtccttccaactta

SEQ ID NO: 385 - RNA, 14 nt, synthetic construct
  source 1..14: mol_type = other RNA
  modified_base 1: mod_base = OTHER; note = 2'-deoxy-2'-fluoro guanosine
  modified_base 2: mod_base = OTHER; note = 2'-deoxy-2'-fluoro adenosine
  modified_base 3: mod_base = OTHER; note = 2'-O-methyl adenosine
  modified_base 4: mod_base = gm
  modified_base 5: mod_base = gm
  modified_base 6: mod_base = OTHER; note = 2'-O-methyl adenosine
  modified_base 7: mod_base = OTHER; note = 2'-deoxy-2'-fluoro cytidine
  modified_base 8: mod_base = cm
  modified_base 9: mod_base = OTHER; note = 2'-deoxy-2'-fluoro adenosine
  modified_base 10: mod_base = cm
  modified_base 11: mod_base = OTHER; note = 2'-O-methyl adenosine
  modified_base 12: mod_base = um
  modified_base 13: mod_base = gm
  modified_base 14: mod_base = cm
  modified_base 12..14: mod_base = OTHER; note = phosphorothioate (PS) linkage
  misc_feature 1: note = The Gf is attached to VPUmsAfsAmGmUmUf[GNA-G]
gaaggaccacatgc

SEQ ID NO: 386 - RNA, 19 nt, synthetic construct
  source 1..19: mol_type = other RNA
  modified_base 1: mod_base = OTHER; note = 2'-O-methyl adenosine
  modified_base 2: mod_base = um
  modified_base 3: mod_base = cm
  modified_base 4: mod_base = OTHER; note = 2'-O-methyl adenosine
  modified_base 5: mod_base = OTHER; note = 2'-deoxy-2'-fluoro adenosine
  modified_base 6: mod_base = gm
  modified_base 7: mod_base = OTHER; note = 2'-deoxy-2'-fluoro guanosine
  modified_base 8: mod_base = OTHER; note = 2'-deoxy-2'-fluoro cytidine
  modified_base 9: mod_base = OTHER; note = 2'-deoxy-2'-fluoro cytidine
  modified_base 10: mod_base = um
  modified_base 11: mod_base = OTHER; note = 2'-O-methyl adenosine
  modified_base 12: mod_base = cm
  modified_base 13: mod_base = um
  modified_base 14: mod_base = gm
  modified_base 15: mod_base = um
  modified_base 16: mod_base = gm
  modified_base 17: mod_base = OTHER; note = 2'-O-methyl adenosine
  modified_base 18: mod_base = cm
  modified_base 19: mod_base = OTHER; note = 2'-O-methyl adenosine
  modified_base 1..3: mod_base = OTHER; note = phosphorothioate (PS) linkage
  modified_base 18^19: mod_base = OTHER; note = phosphorothioate (PS) linkage
  misc_feature 19: note = The Am is attached through the phosphorothioate linkage to [C5Cholesterol]
atcaaggcctactgtgaca

SEQ ID NO: 387 - RNA, 21 nt, synthetic construct
  source 1..21: mol_type = other RNA
  modified_base 1: mod_base = OTHER; note = vinyl phosphonate 2'-O-methyl uridine
  modified_base 2: mod_base = OTHER; note = 2'-deoxy-2'-fluoro guanosine
  modified_base 3: mod_base = um
  modified_base 4: mod_base = cm
  modified_base 5: mod_base = OTHER; note = 2'-O-methyl adenosine
  modified_base 6: mod_base = OTHER; note = 2'-deoxy-2'-fluoro cytidine
  modified_base 7: mod_base = OTHER; note = 2'-O-methyl adenosine
  modified_base 8: mod_base = OTHER; note = 2'-deoxy-2'-fluoro guanosine
  modified_base 9: mod_base = OTHER; note = 2'-deoxy-2'-fluoro uridine
  modified_base 10: mod_base = OTHER; note = 2'-O-methyl adenosine
  modified_base 11: mod_base = gm
  modified_base 12: mod_base = gm
  modified_base 13: mod_base = cm
  modified_base 14: mod_base = OTHER; note = 2'-deoxy-2'-fluoro cytidine
  modified_base 15: mod_base = um
  modified_base 16: mod_base = OTHER; note = 2'-deoxy-2'-fluoro uridine
  modified_base 17: mod_base = gm
  modified_base 18: mod_base = OTHER; note = 2'-O-methyl adenosine
  modified_base 19: mod_base = um
  modified_base 20: mod_base = cm
  modified_base 21: mod_base = um
  modified_base 1..3: mod_base = OTHER; note = phosphorothioate (PS) linkage
  modified_base 19..21: mod_base = OTHER; note = phosphorothioate (PS) linkage
tgtcacagtaggccttgatct

SEQ ID NO: 388 - RNA, 19 nt, synthetic construct
  source 1..19: mol_type = other RNA
  modified_base 1: mod_base = OTHER; note = 2'-O-methyl adenosine
  modified_base 2: mod_base = um
  modified_base 3: mod_base = cm
  modified_base 4: mod_base = OTHER; note = 2'-O-methyl adenosine
  modified_base 5: mod_base = OTHER; note = 2'-deoxy-2'-fluoro adenosine
  modified_base 6: mod_base = gm
  modified_base 7: mod_base = OTHER; note = 2'-deoxy-2'-fluoro guanosine
  modified_base 8: mod_base = OTHER; note = 2'-deoxy-2'-fluoro cytidine
  modified_base 9: mod_base = OTHER; note = 2'-deoxy-2'-fluoro cytidine
  modified_base 10: mod_base = um
  modified_base 11: mod_base = OTHER; note = 2'-O-methyl adenosine
  modified_base 12: mod_base = cm
  modified_base 13: mod_base = um
  modified_base 14: mod_base = gm
  modified_base 15: mod_base = um
  modified_base 16: mod_base = gm
  modified_base 17: mod_base = OTHER; note = 2'-O-methyl adenosine
  modified_base 18: mod_base = cm
  modified_base 19: mod_base = OTHER; note = 2'-O-methyl adenosine
  modified_base 1..3: mod_base = OTHER; note = phosphorothioate (PS) linkage
  modified_base 18^19: mod_base = OTHER; note = phosphorothioate (PS) linkage
  misc_feature 19: note = The Am is attached through the phosphorothioate linkage to [C5Cholesterol]
atcaaggcctactgtgaca

SEQ ID NO: 389 - RNA, 16 nt, synthetic construct
  source 1..16: mol_type = other RNA
  modified_base 1: mod_base = OTHER; note = 2'-deoxy-2'-fluoro cytidine
  modified_base 2: mod_base = OTHER; note = 2'-O-methyl adenosine
  modified_base 3: mod_base = OTHER; note = 2'-deoxy-2'-fluoro guanosine
  modified_base 4: mod_base = OTHER; note = 2'-deoxy-2'-fluoro uridine
  modified_base 5: mod_base = OTHER; note = 2'-O-methyl adenosine
  modified_base 6: mod_base = gm
  modified_base 7: mod_base = gm
  modified_base 8: mod_base = cm
  modified_base 9: mod_base = OTHER; note = 2'-deoxy-2'-fluoro cytidine
  modified_base 10: mod_base = um
  modified_base 11: mod_base = OTHER; note = 2'-deoxy-2'-fluoro uridine
  modified_base 12: mod_base = gm
  modified_base 13: mod_base = OTHER; note = 2'-O-methyl adenosine
  modified_base 14: mod_base = um
  modified_base 15: mod_base = cm
  modified_base 16: mod_base = um
  misc_feature 1: note = The Cf is attached to VPUmsGfsUmCm[N3T1-A-R]
  modified_base 14..16: mod_base = OTHER; note = phosphorothioate (PS) linkage
cagtaggccttgatct

SEQ ID NO: 390 - RNA, 19 nt, synthetic construct
  source 1..19: mol_type = other RNA
  modified_base 1: mod_base = OTHER; note = 2'-O-methyl adenosine
  modified_base 2: mod_base = um
  modified_base 3: mod_base = cm
  modified_base 4: mod_base = OTHER; note = 2'-O-methyl adenosine
  modified_base 5: mod_base = OTHER; note = 2'-deoxy-2'-fluoro adenosine
  modified_base 6: mod_base = gm
  modified_base 7: mod_base = OTHER; note = 2'-deoxy-2'-fluoro guanosine
  modified_base 8: mod_base = OTHER; note = 2'-deoxy-2'-fluoro cytidine
  modified_base 9: mod_base = OTHER; note = 2'-deoxy-2'-fluoro cytidine
  modified_base 10: mod_base = um
  modified_base 11: mod_base = OTHER; note = 2'-O-methyl adenosine
  modified_base 12: mod_base = cm
  modified_base 13: mod_base = um
  modified_base 14: mod_base = gm
  modified_base 15: mod_base = um
  modified_base 16: mod_base = gm
  modified_base 17: mod_base = OTHER; note = 2'-O-methyl adenosine
  modified_base 18: mod_base = cm
  modified_base 19: mod_base = OTHER; note = 2'-O-methyl adenosine
  modified_base 1..3: mod_base = OTHER; note = phosphorothioate (PS) linkage
  modified_base 18^19: mod_base = OTHER; note = phosphorothioate (PS) linkage
  misc_feature 19: note = The Am is attached through the phosphorothioate linkage to [C5Cholesterol]
atcaaggcctactgtgaca

SEQ ID NO: 391 - RNA, 14 nt, synthetic construct
  source 1..14: mol_type = other RNA
  modified_base 1: mod_base = OTHER; note = 2'-deoxy-2'-fluoro guanosine
  modified_base 2: mod_base = OTHER; note = 2'-deoxy-2'-fluoro uridine
  modified_base 3: mod_base = OTHER; note = 2'-O-methyl adenosine
  modified_base 4: mod_base = gm
  modified_base 5: mod_base = gm
  modified_base 6: mod_base = cm
  modified_base 7: mod_base = OTHER; note = 2'-deoxy-2'-fluoro cytidine
  modified_base 8: mod_base = um
  modified_base 9: mod_base = OTHER; note = 2'-deoxy-2'-fluoro uridine
  modified_base 10: mod_base = gm
  modified_base 11: mod_base = OTHER; note = 2'-O-methyl adenosine
  modified_base 12: mod_base = um
  modified_base 13: mod_base = cm
  modified_base 14: mod_base = um
  misc_feature 1: note = The Gf is attached to VPUmsGfsUmCmAmCf[N3T1-A-R]
  modified_base 12..14: mod_base = OTHER; note = phosphorothioate (PS) linkage
gtaggccttgatct

SEQ ID NO: 392 - RNA, 19 nt, synthetic construct
  source 1..19: mol_type = other RNA
  modified_base 1: mod_base = OTHER; note = 2'-O-methyl adenosine
  modified_base 2: mod_base = um
  modified_base 3: mod_base = cm
  modified_base 4: mod_base = OTHER; note = 2'-O-methyl adenosine
  modified_base 5: mod_base = OTHER; note = 2'-deoxy-2'-fluoro adenosine
  modified_base 6: mod_base = gm
  modified_base 7: mod_base = OTHER; note = 2'-deoxy-2'-fluoro guanosine
  modified_base 8: mod_base = OTHER; note = 2'-deoxy-2'-fluoro cytidine
  modified_base 9: mod_base = OTHER; note = 2'-deoxy-2'-fluoro cytidine
  modified_base 10: mod_base = um
  modified_base 11: mod_base = OTHER; note = 2'-O-methyl adenosine
  modified_base 12: mod_base = cm
  modified_base 13: mod_base = um
  modified_base 14: mod_base = gm
  modified_base 15: mod_base = um
  modified_base 16: mod_base = gm
  modified_base 17: mod_base = OTHER; note = 2'-O-methyl adenosine
  modified_base 18: mod_base = cm
  modified_base 19: mod_base = OTHER; note = 2'-O-methyl adenosine
  modified_base 1..3: mod_base = OTHER; note = phosphorothioate (PS) linkage
  modified_base 18^19: mod_base = OTHER; note = phosphorothioate (PS) linkage
  misc_feature 19: note = The Am is attached through the phosphorothioate linkage to [C5Cholesterol]
atcaaggcctactgtgaca

SEQ ID NO: 393 - RNA, 14 nt, synthetic construct
  source 1..14: mol_type = other RNA
  modified_base 1: mod_base = OTHER; note = 2'-deoxy-2'-fluoro guanosine
  modified_base 2: mod_base = OTHER; note = 2'-deoxy-2'-fluoro uridine
  modified_base 3: mod_base = OTHER; note = 2'-O-methyl adenosine
  modified_base 4: mod_base = gm
  modified_base 5: mod_base = gm
  modified_base 6: mod_base = cm
  modified_base 7: mod_base = OTHER; note = 2'-deoxy-2'-fluoro cytidine
  modified_base 8: mod_base = um
  modified_base 9: mod_base = OTHER; note = 2'-deoxy-2'-fluoro uridine
  modified_base 10: mod_base = gm
  modified_base 11: mod_base = OTHER; note = 2'-O-methyl adenosine
  modified_base 12: mod_base = um
  modified_base 13: mod_base = cm
  modified_base 14: mod_base = um
  misc_feature 1: note = The Gf is attached to VPUmsGfsUmCmAmCf[GNA-A]
  modified_base 12..14: mod_base = OTHER; note = phosphorothioate (PS) linkage
gtaggccttgatct

SEQ ID NO: 394 - RNA, 19 nt, synthetic construct
  source 1..19: mol_type = other RNA
  modified_base 1: mod_base = OTHER; note = 2'-O-methyl adenosine
  modified_base 2: mod_base = gm
  modified_base 3: mod_base = OTHER; note = 2'-O-methyl adenosine
  modified_base 4: mod_base = gm
  modified_base 5: mod_base = OTHER; note = 2'-deoxy-2'-fluoro adenosine
  modified_base 6: mod_base = OTHER; note = 2'-O-methyl adenosine
  modified_base 7: mod_base = OTHER; note = 2'-deoxy-2'-fluoro adenosine
  modified_base 8: mod_base = OTHER; note = 2'-deoxy-2'-fluoro adenosine
  modified_base 9: mod_base = OTHER; note = 2'-deoxy-2'-fluoro guanosine
  modified_base 10: mod_base = OTHER; note = 2'-O-methyl adenosine
  modified_base 11: mod_base = gm
  modified_base 12: mod_base = OTHER; note = 2'-O-methyl adenosine
  modified_base 13: mod_base = OTHER; note = 2'-O-methyl adenosine
  modified_base 14: mod_base = OTHER; note = 2'-O-methyl adenosine
  modified_base 15: mod_base = gm
  modified_base 16: mod_base = um
  modified_base 17: mod_base = gm
  modified_base 18: mod_base = um
  modified_base 19: mod_base = um
  modified_base 1..3: mod_base = OTHER; note = phosphorothioate (PS) linkage
  modified_base 18^19: mod_base = OTHER; note = phosphorothioate (PS) linkage
  misc_feature 19: note = The Um is attached through the phosphorothioate linkage to [C5Cholesterol]
agagaaaagagaaagtgtt

SEQ ID NO: 395 - RNA, 21 nt, synthetic construct
  source 1..21: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = cm
  modified_base 4: mod_base = OTHER; note = 2'-O-methyl adenosine
  modified_base 5: mod_base = cm
  modified_base 6: mod_base = OTHER; note = 2'-deoxy-2'-fluoro uridine
  modified_base 7: mod_base = um
  modified_base 8: mod_base = OTHER; note = 2'-deoxy-2'-fluoro uridine
  modified_base 9: mod_base = OTHER; note = 2'-deoxy-2'-fluoro cytidine
  modified_base 10: mod_base = um
  modified_base 11: mod_base = cm
  modified_base 12: mod_base = um
  modified_base 13: mod_base = um
  modified_base 14: mod_base = OTHER; note = 2'-deoxy-2'-fluoro uridine
  modified_base 15: mod_base = um
  modified_base 16: mod_base = OTHER; note = 2'-deoxy-2'-fluoro cytidine
  modified_base 17: mod_base = um
  modified_base 18: mod_base = cm
  modified_base 19: mod_base = um
  modified_base 20: mod_base = um
  modified_base 21: mod_base = um
  modified_base 1..3: mod_base = OTHER; note = phosphorothioate (PS) linkage
  modified_base 19..21: mod_base = OTHER; note = phosphorothioate (PS) linkage
tacactttctcttttctcttt

SEQ ID NO: 396 - RNA, 19 nt, synthetic construct
  source 1..19: mol_type = other RNA
  modified_base 1: mod_base = OTHER; note = 2'-O-methyl adenosine
  modified_base 2: mod_base = gm
  modified_base 3: mod_base = OTHER; note = 2'-O-methyl adenosine
  modified_base 4: mod_base = gm
  modified_base 5: mod_base = OTHER; note = 2'-deoxy-2'-fluoro adenosine
  modified_base 6: mod_base = OTHER; note = 2'-O-methyl adenosine
  modified_base 7: mod_base = OTHER; note = 2'-deoxy-2'-fluoro adenosine
  modified_base 8: mod_base = OTHER; note = 2'-deoxy-2'-fluoro adenosine
  modified_base 9: mod_base = OTHER; note = 2'-deoxy-2'-fluoro guanosine
  modified_base 10: mod_base = OTHER; note = 2'-O-methyl adenosine
  modified_base 11: mod_base = gm
  modified_base 12: mod_base = OTHER; note = 2'-O-methyl adenosine
  modified_base 13: mod_base = OTHER; note = 2'-O-methyl adenosine
  modified_base 14: mod_base = OTHER; note = 2'-O-methyl adenosine
  modified_base 15: mod_base = gm
  modified_base 16: mod_base = um
  modified_base 17: mod_base = gm
  modified_base 18: mod_base = um
  modified_base 19: mod_base = um
  modified_base 1..3: mod_base = OTHER; note = phosphorothioate (PS) linkage
  modified_base 18^19: mod_base = OTHER; note = phosphorothioate (PS) linkage
  misc_feature 19: note = The Um is attached through the phosphorothioate linkage to [C5Cholesterol]
agagaaaagagaaagtgtt

SEQ ID NO: 397 - RNA, 14 nt, synthetic construct
  source 1..14: mol_type = other RNA
  modified_base 1: mod_base = OTHER; note = 2'-deoxy-2'-fluoro uridine
  modified_base 2: mod_base = OTHER; note = 2'-deoxy-2'-fluoro cytidine
  modified_base 3: mod_base = um
  modified_base 4: mod_base = cm
  modified_base 5: mod_base = um
  modified_base 6: mod_base = um
  modified_base 7: mod_base = OTHER; note = 2'-deoxy-2'-fluoro uridine
  modified_base 8: mod_base = um
  modified_base 9: mod_base = OTHER; note = 2'-deoxy-2'-fluoro cytidine
  modified_base 10: mod_base = um
  modified_base 11: mod_base = cm
  modified_base 12: mod_base = um
  modified_base 13: mod_base = um
  modified_base 14: mod_base = um
  misc_feature 1: note = The Uf is attached to VPUmsAfsCmAmCmUf[N3T1-U-R]
  modified_base 12..14: mod_base = OTHER; note = phosphorothioate (PS) linkage
tctcttttctcttt

SEQ ID NO: 398 - RNA, 19 nt, synthetic construct
  source 1..19: mol_type = other RNA
  modified_base 1: mod_base = OTHER; note = 2'-O-methyl adenosine
  modified_base 2: mod_base = um
  modified_base 3: mod_base = gm
  modified_base 4: mod_base = um
  modified_base 5: mod_base = OTHER; note = 2'-deoxy-2'-fluoro guanosine
  modified_base 6: mod_base = gm
  modified_base 7: mod_base = OTHER; note = 2'-deoxy-2'-fluoro uridine
  modified_base 8: mod_base = OTHER; note = 2'-deoxy-2'-fluoro cytidine
  modified_base 9: mod_base = OTHER; note = 2'-deoxy-2'-fluoro cytidine
  modified_base 10: mod_base = um
  modified_base 11: mod_base = um
  modified_base 12: mod_base = cm
  modified_base 13: mod_base = cm
  modified_base 14: mod_base = OTHER; note = 2'-O-methyl adenosine
  modified_base 15: mod_base = OTHER; note = 2'-O-methyl adenosine
  modified_base 16: mod_base = cm
  modified_base 17: mod_base = um
  modified_base 18: mod_base = um
  modified_base 19: mod_base = OTHER; note = 2'-O-methyl adenosine
  modified_base 1..3: mod_base = OTHER; note = phosphorothioate (PS) linkage
  modified_base 18^19: mod_base = OTHER; note = phosphorothioate (PS) linkage
  misc_feature 19: note = The Am is attached through the phosphorothioate linkage to (C5Cholesterol)
atgtggtccttccaactta

SEQ ID NO: 399 - RNA, 21 nt, synthetic construct
  source 1..21: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = OTHER; note = 2'-O-methyl adenosine
  modified_base 4: mod_base = gm
  modified_base 5: mod_base = um
  modified_base 6: mod_base = OTHER; note = 2'-deoxy-2'-fluoro uridine
  modified_base 7: mod_base = gm
  modified_base 8: mod_base = OTHER; note = 2'-deoxy-2'-fluoro guanosine
  modified_base 9: mod_base = OTHER; note = 2'-deoxy-2'-fluoro adenosine
  modified_base 10: mod_base = OTHER; note = 2'-O-methyl adenosine
  modified_base 11: mod_base = gm
  modified_base 12: mod_base = gm
  modified_base 13: mod_base = OTHER; note = 2'-O-methyl adenosine
  modified_base 14: mod_base = OTHER; note = 2'-deoxy-2'-fluoro cytidine
  modified_base 15: mod_base = cm
  modified_base 16: mod_base = OTHER; note = 2'-deoxy-2'-fluoro adenosine
  modified_base 17: mod_base = cm
  modified_base 18: mod_base = OTHER; note = 2'-O-methyl adenosine
  modified_base 19: mod_base = um
  modified_base 20: mod_base = gm
  modified_base 21: mod_base = cm
  modified_base 1..3: mod_base = OTHER; note = phosphorothioate (PS) linkage
  modified_base 19..21: mod_base = OTHER; note = phosphorothioate (PS) linkage
taagttggaaggaccacatgc

SEQ ID NO: 400 - RNA, 19 nt, synthetic construct
  source 1..19: mol_type = other RNA
  modified_base 1: mod_base = OTHER; note = 2'-O-methyl adenosine
  modified_base 2: mod_base = um
  modified_base 3: mod_base = gm
  modified_base 4: mod_base = um
  modified_base 5: mod_base = OTHER; note = 2'-deoxy-2'-fluoro guanosine
  modified_base 6: mod_base = gm
  modified_base 7: mod_base = OTHER; note = 2'-deoxy-2'-fluoro uridine
  modified_base 8: mod_base = OTHER; note = 2'-deoxy-2'-fluoro cytidine
  modified_base 9: mod_base = OTHER; note = 2'-deoxy-2'-fluoro cytidine
  modified_base 10: mod_base = um
  modified_base 11: mod_base = um
  modified_base 12: mod_base = cm
  modified_base 13: mod_base = cm
  modified_base 14: mod_base = OTHER; note = 2'-O-methyl adenosine
  modified_base 15: mod_base = OTHER; note = 2'-O-methyl adenosine
  modified_base 16: mod_base = cm
  modified_base 17: mod_base = um
  modified_base 18: mod_base = um
  modified_base 19: mod_base = OTHER; note = 2'-O-methyl adenosine
  modified_base 1..3: mod_base = OTHER; note = phosphorothioate (PS) linkage
  modified_base 18^19: mod_base = OTHER; note = phosphorothioate (PS) linkage
  misc_feature 19: note = The Am is attached through the phosphorothioate linkage to [C5Cholesterol]
atgtggtccttccaactta

SEQ ID NO: 401 - RNA, 14 nt, synthetic construct
  source 1..14: mol_type = other RNA
  modified_base 1: mod_base = OTHER; note = 2'-deoxy-2'-fluoro guanosine
  modified_base 2: mod_base = OTHER; note = 2'-deoxy-2'-fluoro adenosine
  modified_base 3: mod_base = OTHER; note = 2'-O-methyl adenosine
  modified_base 4: mod_base = gm
  modified_base 5: mod_base = gm
  modified_base 6: mod_base = OTHER; note = 2'-O-methyl adenosine
  modified_base 7: mod_base = OTHER; note = 2'-deoxy-2'-fluoro cytidine
  modified_base 8: mod_base = cm
  modified_base 9: mod_base = OTHER; note = 2'-deoxy-2'-fluoro adenosine
  modified_base 10: mod_base = cm
  modified_base 11: mod_base = OTHER; note = 2'-O-methyl adenosine
  modified_base 12: mod_base = um
  modified_base 13: mod_base = gm
  modified_base 14: mod_base = cm
  misc_feature 1: note = The Gf is attached to VPUmsAfsAmGmUmUf[N3T1-G-R]
  modified_base 12..14: mod_base = OTHER; note = phosphorothioate (PS) linkage
gaaggaccacatgc

SEQ ID NO: 402 - RNA, 19 nt, synthetic construct
  source 1..19: mol_type = other RNA
  modified_base 1: mod_base = OTHER; note = 2'-O-methyl adenosine
  modified_base 2: mod_base = gm
  modified_base 3: mod_base = OTHER; note = 2'-O-methyl adenosine
  modified_base 4: mod_base = gm
  modified_base 5: mod_base = OTHER; note = 2'-deoxy-2'-fluoro adenosine
  modified_base 6: mod_base = OTHER; note = 2'-O-methyl adenosine
  modified_base 7: mod_base = OTHER; note = 2'-deoxy-2'-fluoro adenosine
  modified_base 8: mod_base = OTHER; note = 2'-deoxy-2'-fluoro adenosine
  modified_base 9: mod_base = OTHER; note = 2'-deoxy-2'-fluoro guanosine
  modified_base 10: mod_base = OTHER; note = 2'-O-methyl adenosine
  modified_base 11: mod_base = gm
  modified_base 12: mod_base = OTHER; note = 2'-O-methyl adenosine
  modified_base 13: mod_base = OTHER; note = 2'-O-methyl adenosine
  modified_base 14: mod_base = OTHER; note = 2'-O-methyl adenosine
  modified_base 15: mod_base = gm
  modified_base 16: mod_base = um
  modified_base 17: mod_base = gm
  modified_base 18: mod_base = um
  modified_base 19: mod_base = um
  modified_base 1..3: mod_base = OTHER; note = phosphorothioate (PS) linkage
  modified_base 18^19: mod_base = OTHER; note = phosphorothioate (PS) linkage
  misc_feature 19: note = The Um is attached through the phosphorothioate linkage to [N3T12-024-01]
agagaaaagagaaagtgtt

SEQ ID NO: 403 - RNA, 21 nt, synthetic construct
  source 1..21: mol_type = other RNA
  modified_base 1: mod_base = OTHER; note = vinyl phosphonate 2'-O-methyl uridine
  modified_base 2: mod_base = OTHER; note = 2'-deoxy-2'-fluoro adenosine
  modified_base 3: mod_base = cm
  modified_base 4: mod_base = OTHER; note = 2'-O-methyl adenosine
  modified_base 5: mod_base = cm
  modified_base 6: mod_base = OTHER; note = 2'-deoxy-2'-fluoro uridine
  modified_base 7: mod_base = um
  modified_base 8: mod_base = OTHER; note = 2'-deoxy-2'-fluoro uridine
  modified_base 9: mod_base = OTHER; note = 2'-deoxy-2'-fluoro cytidine
  modified_base 10: mod_base = um
  modified_base 11: mod_base = cm
  modified_base 12: mod_base = um
  modified_base 13: mod_base = um
  modified_base 14: mod_base = OTHER; note = 2'-deoxy-2'-fluoro uridine
  modified_base 15: mod_base = um
  modified_base 16: mod_base = OTHER; note = 2'-deoxy-2'-fluoro cytidine
  modified_base 17: mod_base = um
  modified_base 18: mod_base = cm
  modified_base 19: mod_base = um
  modified_base 20: mod_base = um
  modified_base 21: mod_base = um
  modified_base 1..3: mod_base = OTHER; note = phosphorothioate (PS) linkage
  modified_base 19..21: mod_base = OTHER; note = phosphorothioate (PS) linkage
tacactttctcttttctcttt

SEQ ID NO: 404 - DNA, 20 bp, synthetic construct
  source 1..20: mol_type = other DNA
  modified_base 1: mod_base = OTHER; note = 2'-O-(2-methoxyethyl) adenosine
  modified_base 2: mod_base = OTHER; note = 2'-O-(2-methoxyethyl)-5-methylcytidine
  modified_base 3: mod_base = OTHER; note = 2'-O-(2-methoxyethyl) guanosine
  modified_base 4: mod_base = OTHER; note = 2'-O-(2-methoxyethyl)-5-methylcytidine
  modified_base 5: mod_base = OTHER; note = 2'-O-(2-methoxyethyl) adenosine
  modified_base 16: mod_base = OTHER; note = 2'-O-(2-methoxyethyl) thymidine
  modified_base 17: mod_base = OTHER; note = 2'-O-(2-methoxyethyl)-5-methylcytidine
  modified_base 18: mod_base = OTHER; note = 2'-O-(2-methoxyethyl) adenosine
  modified_base 19: mod_base = OTHER; note = 2'-O-(2-methoxyethyl) thymidine
  modified_base 20: mod_base = OTHER; note = 2'-O-(2-methoxyethyl) adenosine
acgcacttaacaatatcata

SEQ ID NO: 405 - DNA, 20 bp, synthetic construct
  source 1..20: mol_type = other DNA
  modified_base 1: mod_base = OTHER; note = 2'-O-(2-methoxyethyl) adenosine
  modified_base 2: mod_base = OTHER; note = 2'-O-(2-methoxyethyl)-5-methylcytidine
  modified_base 3: mod_base = OTHER; note = 2'-O-(2-methoxyethyl) guanosine
  modified_base 4: mod_base = OTHER; note = 2'-O-(2-methoxyethyl)-5-methylcytidine
  modified_base 5: mod_base = OTHER; note = 2'-O-(2-methoxyethyl) adenosine
  modified_base 16: mod_base = OTHER; note = 2'-O-(2-methoxyethyl) thymidine
  modified_base 17: mod_base = OTHER; note = 2'-O-(2-methoxyethyl)-5-methylcytidine
  modified_base 18: mod_base = OTHER; note = 2'-O-(2-methoxyethyl) adenosine
  modified_base 19: mod_base = OTHER; note = 2'-O-(2-methoxyethyl) thymidine
  modified_base 20: mod_base = OTHER; note = 2'-O-(2-methoxyethyl) adenosine
  misc_feature 20: note = The moeA is attached to [N3T12-012-01]
acgcacttaacaatatcata

(19) United States
(12) Patent Application Publication
(10) Pub. No.: US 2026/0327626 A1
(43) Pub. Date: Oct. 22, 2026

(54) NOVEL SIRNA CONSTRUCTS, THERAPEUTICS, AND MODIFICATIONS

(71) Applicants: NEURO3 THERAPEUTICS, INC., Cambridge, MA (US); NEURO3 THERAPEUTICS (SUZHOU) LTD., Suzhou (CN)

(72) Inventors: Min LUO, Suzhou (CN); Zheng LI, Cambridge, MA (US); Xinyan HUANG, Cambridge, MA (US)

(21) Appl. No.: 19/573,791

(22) Filed: Mar. 20, 2026

Related U.S. Application Data
(63) Continuation of application No. PCT/CN2024/121490, filed on Sep. 26, 2024.

(30) Foreign Application Priority Data
Sep. 27, 2023 (WO) PCT/CN2023/122067

(51) Int. Cl. C12N 15/113

(52) U.S. Cl. CPC C12N 15/1137; C12Y 207/11001; C12N 2310/14; C12N 2310/315; C12N 2310/321; C12N 2310/322

(57) ABSTRACT

This disclosure provides optionally modified small interfering ribonucleic acid (siRNA) compounds, pharmaceutical compositions, and methods of use of same. The optionally modified siRNA compounds may be tailored for improved delivery to the central nervous system, reduced off-target effects, and/or reducing the expression, i.e., translation into protein, of mRNA transcribed from a gene of interest, e.g. LRRK2, VEGFA, or ANGPT2. In some embodiments, the sense strand of the siRNA comprises a synthetic nucleotide and/or a lipid conjugated to one or both ends or within any positions of the sense strand.

### CROSS REFERENCE TO RELATED APPLICATIONS

This application is a continuation application of International Application No. PCT/CN2024/121490, filed on Sep. 26, 2024, which claims the benefit of PCT Application No. PCT/CN2023/122067, filed on Sep. 27, 2023, the contents of each of which are incorporated herein by reference.

### REFERENCE TO A SEQUENCE LISTING

The contents of the electronic sequence listing (P2024TC3029.xml; Size 1,509,734 bytes; and Date of Creation: Jun. 15, 2026) is herein incorporated by reference in its entirety.

### FIELD

The disclosure relates generally to the field of synthetic biology, and more specifically to siRNA oligonucleotides and uses thereof.

### BACKGROUND

Small interfering ribonucleic acids (siRNA), sometimes referred to as short interfering ribonucleic acids or RNAi agents (e.g. siRNA, dsRNA, antisense oligonucleotide, etc.), have recently been found to be useful as therapeutic agents in the treatment of genetic disease and/or gene-associated diseases, such as diseases that are undruggable through more traditional therapeutic means. Without being bound by theory, siRNAs are thought to arise from a natural phenomenon in which small (i.e., about 15 to about 30 nucleotide) non-coding RNAs have been found to regulate genes and genomes through a mechanism known as RNA interference (RNAi), in which a double-stranded RNA (dsRNA) or antisense oligonucleotide induces gene silencing by targeting complementary mRNA for degradation. More specifically, it is believed that the RNase III-like endo-ribonuclease enzyme termed Dicer cleaves long dsRNAs or hairpin RNAs to form siRNAs, which generally comprise a short region of dsRNA wherein each strand of RNA is phosphorylated on the 5′ end and contains an about 2 nucleotide overhang on the 3′ end. siRNAs within a cell encounter and are bound by a multi-protein component complex of the argonaute family (Ago2) termed the RNA-induced silencing complex (RISC). Upon binding, the siRNA strand with the less thermodynamically stable 5′ end is typically integrated into the activated RISC complex. The antisense single-stranded siRNA component then guides and aligns the RISC complex on the complementary mRNA. Once the RISC complex finds a complementary section between its antisense siRNA strand and the mRNA sequence, the catalytic function of the RISC enzyme cleaves the mRNA. This cleavage of the mRNA renders the mRNA incapable of further translation into protein and is, instead, marked for degradation. This mechanism acts to effectively silence the gene with a sequence complementary to the siRNA sequence. The utility of siRNA as a therapeutic, however, is limited by its instability, rapid metabolism, and elimination in vivo, as well as by off-target effects and the difficulties of delivering a charged molecule, e.g., an oligonucleotide, to the cytoplasm of a target cell. These difficulties are further enhanced when the target cell is isolated by the blood-brain barrier.

Regarding off-target effects as a challenge in the development of siRNA therapeutics, one of the off-target effects of siRNAs is the mi-RNA-like effect. (Lam et al., (2015) Molecular Therapy Nucleic Acids (2015, 4, e252). The miRNA recognizes a target gene primarily through base-pairing between the seed region (e.g., positions 2-9 from the 5′-end) and the target mRNA for gene suppression. The off-target effects caused by siRNA originate from base-complementarity of the seed regions of the RISC-loaded antisense strand of siRNA with one or more mRNA. The miRNA-like off-target effects of siRNAs have been reported in several studies, and effect expression of a multitude of genes depending on sequences of the seed regions and are serious enough to cause up to 30% of the positive hits in siRNA-based phenotype screening. Additionally, in the case of miRNAs, they are also reported to silence target genes through compensatory pairings within their 3′-end regions (3′-compensatory pairing) when the interactions between seed region and targets become weak, implicating that the miRNA-like off-target effects are likely mediated by such a mechanism.

The Dual Luciferase Report Gene Assay is one of the standard methods used for screening off-target effects for siRNA therapeutics. The dual luciferase reporter gene assay is a powerful tool for quantifying gene expression. The assay involves co-transfecting cells with two different luciferase genes, each under the control of a different promoter. One promoter is constitutive, driving the expression of a reference luciferase (e.g., Renilla luciferase), while the other is under the control of a gene of interest. By measuring the ratio of the two luciferases, researchers can accurately assess changes in gene expression.

There is an ongoing effort to eliminate or reduce miRNA-like off-target effects of siRNAs by modulating siRNA design by judicious application of chemical modifications without compromising the gene silencing efficacy of siRNA therapeutics. This disclosure is directed, in part, to that effort.

The leucine-rich repeat kinase 2 (LRRK2) gene encodes the protein Lrrk2, also known as dardarin or PARK8. Lrrk2 includes kinase and GTPase domains and is expressed in many organs and tissues throughout the body, including the kidneys, lung, heart, liver, and central nervous system. Lrrk2 phosphorylates a broad range of proteins involved in multiple processes such as neuronal plasticity, innate immunity, autophagy, and vesicle trafficking, and it is believed to have a role in signal transduction and cytoskeletal assembly. The LRRK2 gene is located in the chromosomal region 12q11.2-12q13.1 and is highly conserved across a wide range of organisms. The LRRK2 gene is composed of 51 exons encoding 2527 amino acids, which comprise enzymatic domains including a ROC (Ras of complex) domain, a GTPase domain, a serine/threonine kinase domain, a leucine-rich repeat (LRR) domain, a C-terminal WD40 repeat domain, an armadillo repeat (ARM) domain, and an ankyrin repeat (ANK) domain. Mutations in LRRK2 have been found to be associated with inflammatory bowel disease (IBD), Crohn's disease (CD), ulcerative colitis (UC), susceptibility to bacterial infections such as Hansen's disease/leprosy, and Parkinson's disease (PD).

Parkinson's disease (PD) is a progressively debilitating neurodegenerative syndrome that is ultimately fatal. PD can clinically present as tremors, loss of balance and coordination, stiffness, slowing of movement, changes in speech, and cognitive decline. The motor symptoms of PD are believed to result from the death of nerve cells in the substantia nigra, a region of the midbrain that supplies dopamine to the basal ganglia. The progression of the disease corresponds to the aggregation of the alpha-synuclein protein into Lewy bodies within the neurons. There is currently no cure or disease-modifying therapy approved for PD, and available treatment focuses on supplementing dopamine function using dopamine precursors (carbidopa or levodopa), monoamine oxidase inhibitors to reduce dopamine metabolism, and/or dopamine receptor agonists. These treatments, however, become less effective as the disease progresses, and it becomes increasingly difficult to modulate dopaminergic stimulation as the dosages are increased to compensate for dopamine deficit due to the progression of the disease, and these drugs may have serious side effects, including movement disorders, GI disorders, cognitive difficulties, and hallucinations.

LRRK2 mutations are found in both inherited forms of PD (autosomal-dominant parkinsonism), as well as sporadic PD cases. The similar LRRK2 mutations found in both familial and sporadic PD cases suggests that these missense and/or deletion mutations play a critical role in PD etiology. The mutations in LRRK2 associated to PD are generally associated with enhanced kinase and/or GTPase activity. Some of the single nucleotide polymorphisms (SNPs) most highly associated with the development of PD include 2 missense mutations in the kinase domain of LRRK2, i.e., G2019S and I2020T, and 3 missense mutations in the GTPase domain of LRRK2, i.e., R1441C, R1441G, and R1441H. All five of these mutations are affiliated with increased kinase activity of the LRRK2 protein. Moreover, elevated Lrrk2 kinase activity without identified LRRK2 mutations may contribute to idiopathic PD.

Improved methods of treating Parkinson's Disease are needed, as are improved methods of stabilizing and delivering siRNA.

Vascular endothelial growth factor A (Vegf-A) is a protein that stimulates the formation of blood vessels. It is a member of the platelet-derived growth factor family and is essential for both physiological and pathological angiogenesis. Inhibition of Vegf-A is useful to treat certain cancers, e.g. solid tumors and bone marrow cancers, as well as certain degenerative eye conditions, such as wet macular degeneration, the pathology of which is dependent on angiogenesis. Vegf-A inhibitors include monoclonal antibodies such as bevacizumab, sold under the brand name Avastin, which bind Vegf-A and prevent binding of Vegf-A to its receptors (Flt-1 and KDR) on the surface of endothelial cells. Inhibiting Vegf-A inhibits the neovascularization required for tumor growth as well as inhibiting the growth of new lymphatic vessels (lymphangiogenesis) which can facilitate tumor metastasis to distant organs. Inhibiting Vegf-A also controls excess angiogenesis and prevents vision loss in neovascular eye diseases such as wet age-related macular degeneration and macular oedema, which are major causes of vision loss in adults. Avastin is approved for treatment of a variety of solid tumors, including metastatic colorectal cancer, cervical cancer, endometrial cancer, non-small cell lung cancer, glioblastoma, metastatic hepatocellular carcinoma, metastatic renal cell carcinoma, ovarian cancer, primary peritoneal cancer, and fallopian tube cancer. Another anti-Vegf-A mAb, ranibizumab, sold under the brand name Lucentis, is used to treat degenerative diseases of the eye characterized by abnormal neovascularization, e.g. neovascular (wet) age-related macular degeneration (AMD), macular edema following retinal vein occlusion, diabetic macular edema, diabetic retinopathy, and myopic choroidal neovascularization.

Angiopoietin-2 (Angpt-2) is encoded by the ANGPT2 gene, and it is also involved in angiogenesis. Inhibition of Angpt-2 should complement or provide alternatives to current anti-angiogenic strategies in treating cancer and neovascular eye diseases.

Improved methods of treating cancer and neovascular eye diseases and of stabilizing and delivering RNAi agents are needed.

### SUMMARY

We have surprisingly discovered that siRNA corresponding to certain LRRK2 gene sequences is effective to reduce or silence Lrrk2 expression, but siRNA corresponding to other LRRK2 gene sequences is relatively ineffective. By optimizing the targeting sequence, and further, by optimizing and formulating the siRNA to increase stability, reduce off-target effect(s), and enhance delivery, we can provide a therapeutically effective approach to reducing Lrrk2 expression and activity in cells and thereby treat diseases, including PD.

The present disclosure provides compounds, pharmaceutical compositions, and methods of making use of same for reducing LRRK2 expression.

In one aspect, the present disclosure provides a compound (Compound 1), comprising a double-stranded siRNA duplex. In some embodiments, the siRNA duplex comprises one sense and one antisense sequence, wherein at least one of the sense and/or antisense sequence is any one of the sequences of Tables 1-34. In further embodiments, the siRNA duplex comprises a sequence with complementarity to a LRRK2 or VEGFA or ANGPT2 gene, optionally with one or more mutation associated with a disease, disorder, or pathology. In still further embodiments, the siRNA duplex comprises one or more sequence with modifications, such as a phosphorylated 5′ end, a phosphonated 5′ end, a blunt end, at least one nucleotide overhang, an acyclic/unlocked nucleic acid (UNA) and/or a glycol nucleic acid (GNA) motif, a lipophilic moiety, a phosphorothioate linkage, or a modified nucleotide, such as a 2′-deoxy-2′fluoro and/or 2′-O-methyl ribosugar. In some embodiments, the siRNA duplex is part of a pharmaceutical composition, accompanied by a pharmaceutically acceptable diluent or carrier, or is encapsulated within a delivery carrier such as a lipid nanoparticle or modified viral capsid.

In another aspect, the present disclosure provides a method (Method 2), comprising inhibiting expression of a LRRK2 gene, e.g., in a cell or a subject. In some embodiments, the method comprises administration of a siRNA duplex comprising one sense and one antisense sequence, wherein at least one of the sense and/or antisense sequence is any one of the sequences of Tables 1-34. In further embodiments, the method comprises administration of a siRNA duplex comprising a sequence with complementarity to a LRRK2 gene, optionally with one or more mutation associated with a disease, disorder, or pathology. In still further embodiments, the method comprises administration of a siRNA duplex comprising one or more sequence with modifications, as enumerated above. In additional embodiments, the method comprises administering a siRNA, or a pharmaceutical composition of same, to a subject, optionally wherein the subject is a mammal, such as a human. In some embodiments, the method comprises administering a siRNA to a subject that has been diagnosed with a LRRK2-associated disease, disorder, or pathology, for example for example inflammatory bowel disease (IBD), Crohn's disease (CD), ulcerative colitis (UC), susceptibility to bacterial infections such as Hansen's disease/leprosy, and/or Parkinson's disease (PD). In further embodiments, the method comprises administering a siRNA to a subject via intravascular, subcutaneous, intramuscular, intraperitoneal, intracerebral, intracerebroventricular, or intrathecal injection, optionally wherein the siRNA is administered at a dosage of about 0.01 mg/kg to about 50 mg/kg. In still further embodiments, the method comprises wherein the siRNA is administered for the prevention, inhibition, reduction, treatment, and/or amelioration of symptoms associated with a LRRK2-associated disease, disorder, or pathology.

In another embodiment, the disclosure provides a method (Method 2A) of treating a disease or condition characterized by or dependent on angiogenesis or lymphangiogenesis, for example a cancer or ocular neovascular disorder, comprising administering an effective amount of a compound which is a double-stranded siRNA duplex, comprising a sense sequence and a complementary antisense sequence, or an antisense oligonucleotide (ASO) wherein the sequence(s) correspond to a region of a VEGFA or ANGPT2 gene, wherein the siRNA duplex or ASO is effective to significantly reduce VEGFA or ANGPT2 mRNA in a cell.

In another embodiment, the disclosure provides siRNA which is modified to enhance stability and delivery of the siRNA to the cell, by attaching lipid moieties to one or both ends of the sense strand or antisense oligonucleotide (ASO) or within any positions of the sense strand or ASO.

In another embodiment, the disclosure provides siRNA having antisense modifications to reduce off-target effects, as well as novel nucleoside analogues to provide such modifications.

In another embodiment, the disclosure provides siRNA having antisense modifications at the terminus of the construct, to enhance RISC binding, as well as novel nucleoside analogues to provide such modifications.

### BRIEF DESCRIPTION OF THE DRAWINGS

FIG. 1 depicts optional modifications to the RNAi agent to increase efficacy, delivery and stability and reduce off-target effect.

### DETAILED DESCRIPTION

The following description of different embodiments is merely exemplary in nature and is in no way intended to limit the invention, its application, or uses.

### I. Definitions

As used herein, the following terms have the meanings ascribed to them unless specified otherwise.

As used herein, the term “LRRK2” gene is recognized to be equivalent to genes with alternative names but the same underlying sequence, such as DRDN, RIPK7, PARK8, AURA17, ROCO2, and leucine-rich repeat kinase 2, and also to include natural variants of LRRK2, e.g., associated with disease, such as G2019S, I2020T, R1441C, R1441G, R1441H, Y1699C, N2081D, Rs11175593 LRRK2/MUC19, Rs11564258 LRRK2/MUC19, M2397T, R1398H, N551K, Rs1873613, Rs1491938, and R1628P; and particularly including mutations associated with Parkinson's Disease, e.g., at positions 1371, 1437, 1441, 1699, 2019, and 2020, e.g. I1371V, N1437H, N1437D, R1441C, R1441G, R1441H, R1441S, Y1699C, G2019S, and I2020T.

As used herein, sequences comprising a chain of nucleotides using standard nucleotide nomenclature “G”, “C”, “A”, “T”, and “U” are understood to reference guanine, cytosine, adenine, thymidine, and uracil nucleotide bases, either modified or unmodified and with either natural or unnatural linkages. An RNAi agent refers to any molecule that induces RNA interference (RNAi), e.g., siRNA, dsRNA, dsRNA duplex, or antisense oligonucleotide (ASO).

As used herein, gene, DNA, and/or RNA “expression” is understood to refer to progression along the canonical pathway beginning with DNA, which may be transcribed into RNA, which may be translated into protein.

As used herein, “treating” or “treatment” encompasses prophylaxis, mitigation, amelioration of symptoms, or delaying the progression of a disease or condition.

As used herein, “off-target effect(s)” or “off-targets” encompass symptoms associated with the administration and/or presence of an exogeneous therapeutic, e.g., siRNA, arising from interactions with the exogeneous therapeutic at a location other than the site targeted by said therapeutic. For example, off-targets of a siRNA designed to target the LRRK2 gene and/or LRRK2 mRNA would include any symptoms arising from said siRNA binding or interacting with a moiety other than the targeted LRRK2 gene and/or LRRK2 mRNA.

The present disclosure provides compounds, pharmaceutical compositions, and methods of use of same for reducing the expression, i.e., translation into protein, of LRRK2 mRNA transcribed from a LRRK2 gene.

For example, the compounds, pharmaceutical compositions, and methods disclosed are intended to reduce the presence and/or function of Lrrk2 proteins. In certain embodiments, the Lrrk2 protein is a functional, native protein. In other embodiments, the Lrrk2 protein arises from a mutated nucleotide sequence, such that the resultant Lrrk2 protein is active at rates beyond the typical native protein, such as increased kinase activity. In further embodiments, the Lrrk2 protein arises from a mutated nucleotide sequence, such that the resultant Lrrk2 protein is less active than typical native protein or inactive, optionally wherein the Lrrk2 protein is misfolded and/or unable to be efficiently degraded by the endogenous cell metabolism.

In an alternative example, the compounds, pharmaceutical compositions, and methods disclosed are intended to selectively induce the targeted degradation of mRNA transcribed from a LRRK2 gene. In some embodiments, the LRRK2 gene is a healthy, native nucleotide sequence that does not contain any mutations. In other embodiments, the LRRK2 gene contains a mutation, optionally wherein the mutation comprises a missense mutation, one or more single nucleotide polymorphisms (SNPs), a deletion, or combinations thereof, optionally wherein the mutation is located within a coding region and/or noncoding region of the LRRK2 gene. In certain embodiments, the mutation is associated with a pathology, disease state, disease etiology, or disease progression. Examples of LRRK2 gene mutations include, but are not limited to, G2019S, I2020T, R1441C, R1441G, R1441H, Y1699C, N2081D, Rs11175593 LRRK2/MUC19, Rs11564258 LRRK2/MUC19, M2397T, R1398H, N551K, Rs1873613, Rs1491938, and R1628P. Examples of pathologies and/or disease states wherein which the targeted degradation of mRNA transcribed from a LRRK2 gene is expected to be therapeutic include, but are not limited to, inflammatory bowel disease (IBD), Crohn's disease (CD), ulcerative colitis (UC), bacterial infections such as Hansen's disease/leprosy, and Parkinson's disease (PD).

In certain embodiments, the compounds, pharmaceutical compositions, and methods disclosed are functional within a cell, such as one or more cells within a subject. The subject may be a mammal, such as a mouse, rat, dog, cat, pig, cow, sheep, goat, horse, or human. Alternatively, the subject may be a bird, reptile, amphibian, fish, or invertebrate.

In certain embodiments, the present disclosure provides a double stranded siRNA duplex or ASO for inhibiting expression of the LRRK2 gene, wherein the siRNA duplex comprises a sense strand and an antisense strand forming a double stranded region, wherein the sense strand comprises at least 15, at least 17, at least 19 contiguous nucleotides differing by no more than 3 nucleotides from the nucleotide sequences in any one of Tables 1-34.

In certain embodiments, the present disclosure provides a double stranded siRNA duplex or ASO for inhibiting expression of the LRRK2 gene, wherein the siRNA duplex comprises a sense strand and an antisense strand forming a double stranded region, wherein the antisense strand comprises at least 15 contiguous nucleotides differing by no more than 3 nucleotides from the nucleotide sequences in any one of Tables 1-34.

In certain embodiments, the present disclosure provides a double stranded siRNA duplex or

ASO for inhibiting expression of the LRRK2 gene, wherein the siRNA duplex comprises a sense strand and an antisense strand forming a double stranded region, wherein the antisense strand comprises a region of complementarity to an mRNA encoding the Lrrk2 protein, and wherein the region of complementarity comprises at least 15, at least 17, at least 19 contiguous nucleotides differing by no more than 3 nucleotides from any one of the sense and/or antisense nucleotide sequences in any one of Tables 1-34.

In certain embodiments, the present disclosure provides a double stranded siRNA duplex or ASO for inhibiting expression of the LRRK2 gene, wherein the siRNA duplex comprises a sense strand and an antisense strand forming a double stranded region, wherein the double stranded region is at least 21, at least 19, at least 17, at least 15, at least 13 nucleotide bases in length.

In certain embodiments, the present disclosure provides a double stranded siRNA duplex or ASO for inhibiting expression of the LRRK2 gene, wherein the siRNA duplex comprises a sense strand and an antisense strand forming a complementary double stranded region, wherein the sense and/or antisense strand further comprises a blunt end or a non-complementary 3′ overhang of at least 1, at least 2, at least 3, at least 4 nucleotides.

In certain embodiments, the siRNA duplex or ASO inhibits expression of LRRK2 gene by at least 90%, at least 80%, at least 70%, at least 60%, at least 50%, at least 40%, at least 30%, at least 20%, at least 10% relative to control levels and reduce the level of sense- and antisense-containing foci.

In certain embodiments, the present disclosure provides a method of inhibiting expression of a LRRK2 gene in a cell, the method comprising contacting the cell with a RNAi agent of the invention, or a pharmaceutical composition of the invention, thereby inhibiting expression of the LRRK2 gene in the cell. In some embodiments, the cell is in a subject, optionally wherein the subject is a mammal, optionally wherein the subject is human.

In certain embodiments, the present disclosure provides a method of inhibiting the expression of a LRRK2 gene in a cell by at least 90%, at least 80%, at least 70%, at least 60%, at least 50%, at least 40%, at least 30%, at least 20%, at least 10% relative to control levels, the method comprising contacting the cell with a RNAi agent of the disclosure, or a pharmaceutical composition comprising a siRNA duplex or an ASO of the disclosure. In some embodiments, the cell is in a subject, optionally wherein the subject is a mammal, optionally wherein the subject is human.

In certain embodiments, the present disclosure provides a method of inhibiting the expression of LRRK2 mRNA in a cell by at least 90%, at least 80%, 70%, at least 60%, at least 50%, at least 40%, at least 30% at least 20%, at least 10% relative to control levels, the method comprising contacting the cell with a RNAi agent of the invention, or a pharmaceutical composition of the invention. In some embodiments, the cell is in a subject, optionally wherein the subject is a mammal, optionally wherein the subject is human. In further embodiments, the subject has been diagnosed with a LRRK2-related disease, such as inflammatory bowel disease (IBD), Crohn's disease (CD), ulcerative colitis (UC), bacterial infections such as Hansen's disease/leprosy, and Parkinson's disease (PD).

In certain embodiments, the present disclosure provides a method of decreasing the Lrrk2 protein level in serum of a subject by at least 90%, at least 80%, at least 70%, at least 60%, at least 50%, at least 40%, at least 30%, at least 20%, at least 10% relative to control levels, the method comprising administering to the subject a RNAi agent of the invention, or a pharmaceutical composition of the invention.

In certain embodiments, the present disclosure provides a method of treating a subject having a disease, disorder, or pathology that would benefit from reduction in LRRK2 gene expression, comprising administering to the subject a therapeutically effective amount of a RNAi agent of the invention, or a pharmaceutical composition of the invention, thereby treating the subject having the disease, disorder, or pathology that would benefit from reduction in LRRK2 expression.

In certain embodiments, the present disclosure provides a method of ameliorating at least one symptom in a subject having a disease, disorder, or pathology that would benefit from reduction in LRRK2 gene expression, comprising administering to the subject a therapeutically effective amount of a RNAi agent of the invention, or a pharmaceutical composition of the invention, thereby ameliorating and/or reducing at least one symptom in the subject having the disease, disorder, or pathology that would benefit from reduction in LRRK2 expression.

In certain embodiments, the present disclosure provides a method of preventing at least one symptom in a subject having a disease, disorder, or pathology that would benefit from reduction in LRRK2 gene expression, comprising administering to the subject a prophylactically effective amount of a RNAi agent of the invention, or a pharmaceutical composition of the invention, thereby preventing at least one symptom in the subject having the disease, disorder, or pathology that would benefit from reduction in LRRK2 expression.

In certain embodiments, any one of the methods disclosed herein further comprises determining the level of LRRK2 gene, LRRK2 mRNA, and/or Lrrk2 protein in a sample collected from a subject. In some embodiments, the sample is blood, serum, and/or cerebrospinal fluid.

In certain embodiments, the RNAi agent is administered to a subject at a dose of about 0.01 mg/kg to about 50 mg/kg.

In certain embodiments, the RNAi agent is administered by intravenous injection.

In certain embodiments, the RNAi agent is administered to the cerebrospinal fluid, e.g., by intrathecal injection or intracerebroventricular injection (ICV injection).

In certain embodiments, the RNAi agent is delivered using lipid nanoparticles (LNPs). For example, the RNAi agent may be delivered in a lipid nanoparticle formulation, e.g., wherein the lipid nanoparticle comprises the siRNA or ASO surrounded by one or more ionizable lipids (e.g., tertiary amine attached to lipid chains, such as MC3), one or more phospholipids (e.g. DSPC (1,2-distearoyl-sn-glycero-3-phosphocholine), and cholesterol, to form a stable nanoparticle structure, which is coated by one or more pegylated lipids. Within the lipid nanoparticle, the relatively anionic siRNA or ASO is complexed with the relatively cationic ionizable lipid to maintain a stable configuration through administration, which breaks up and releases the RNAi agent upon delivery, e.g. when the lipid nanoparticle reaches the endosome of the target cell. In certain embodiments, the lipid components of the lipid nanoparticle comprise ionizable lipid (e.g., DLin-MC3-DMA), phospholipid (e.g., DSPC), cholesterol and pegylated lipid (e.g., PEG-DMG ((R)-2,3-bis(octadecyloxy) propyl-1-(methoxy polyethylene glycol 2000) carbamate), e.g., at a molar ratio of about 50/10/38.5/1.5.

In certain embodiments, the RNAi agent is delivered using a viral capsid or alternative transfection method. In certain embodiments, the siRNA duplex is a hairpin sequence, i.e., wherein the sense and antisense strands of the siRNA duplex are on a single molecule. In further embodiments, a DNA construct corresponding to the siRNA hairpin sequence is delivered.

In certain embodiments, the RNAi agent comprises a sequence that is complementary to the LRRK2 mRNA sequence. In certain embodiments, the RNAi agent comprises one or more sequence that is or is complementary to one or more sequence listed in Tables 1-34 herein. In further embodiments, the RNAi agent comprises one or more sequence that is of substantial complementarity to one or more sequence listed in Tables 1-34 herein, such as wherein 3 or fewer nucleotides do not match the listed sequence. In certain embodiments, the two strands of the siRNA duplex are mismatched or not completely complementary to each other, such as wherein at least 1, at least 2, at least 3 nucleotides are not complementary to the corresponding nucleotide position within the sense or antisense strand, or where there is an overhang of 0, 1, 2, or 3 nucleotides at either or both ends.

We note, however, that neurons appear to be more amenable than other cell types to taking up a RNAi agent, so where the RNAi agent is delivered directly to the CNS, the RNAi agent may be delivered as naked RNAi agent, or RNAiagent conjugated to a lipid, e.g., 2′-O-hexadecyl (C16) conjugates or the novel lipid conjugates of Compound A.

In certain embodiments, administration of a RNAi agent to a subject causes a decrease, suppression, inhibition, and/or lowering in Lrrk2 protein production, activity, accumulation, and/or aggregation, and/or mRNA levels

In certain embodiments, the RNAi agent comprises one or more sequence that corresponds to one or more hotspot positions on the LRRK2 mRNA sequence, e.g., wherein the hotspots are the sequence sections corresponding to the regions of the mRNA complementary to antisense sequences identified in Tables 1-34, e.g., Tables 1, 4, 5, 7, 13, 20, 22, 26, and/or 30.

In certain embodiments, the siRNA duplex comprises complementary sense and antisense nucleotide sequences having a length of 15-30 nucleotides, optionally 18-25, optionally 18-23, wherein the sequences may be offset by 1, 2 or 3 or more nucleotides and or one strand may be 1, 2, or 3 or more nucleotides longer than the other, so that there is an overhang at one or both ends; for example, wherein the sense strand is 19 nucleotides in length and the antisense strand is 21 nucleotides in length, or the sense strand is 20 nucleotides in length and the antisense strand is 22 nucleotides in length, or the sense strand is 21 nucleotides in length and the antisense strand is 23 nucleotides in length, such that the siRNA duplex has a two nucleotide overhang.

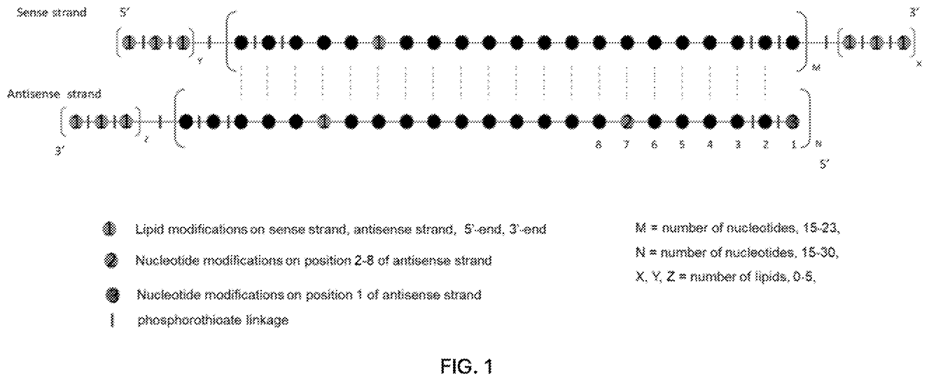

In certain embodiments the RNAi agent (Compound A) comprises one or more of the following modifications as depicted in FIG. 1:
1. RISC loading at position 1 of the antisense (AS) strand (numbering from the 5′-end): Modified nucleosides and analogs are useful for incorporation at one of the terminal positions of an oligomeric compound, e.g. the 5′-end of AS strand or the 3′-end of AS strand. The guide strand of an siRNA duplex may bear a 5′-phosphate or other analogs to bind the effector protein of the RNA-induced silencing complex Argonaute 2 (AGO2).
2. Modifications at seed region (position 2-8) of the AS strand (from 5′-end) to reduce off-target effects: The off-target effects are largely driven by binding of the RISC-loaded siRNA to off-target transcripts mediated through base pairing between the seed region of the siRNA guide strand (nucleotides 2-8) and complementary site(s) in the mRNAs. Thermally destabilizing modifications in the seed region of the siRNA guide strand enhance siRNA specificity, mitigating off-target effects while still allowing for productive full-length on-target recognition.
3. Lipid conjugation on sense strand (SS) or antisense strand (AS): Oligonucleotide-ligand conjugates can facilitate delivery and uptake of the siRNA duplex by the target cells.
4. Terminal phosphorothioate (PS) linkages to increase stability: Design of terminal phosphorothioate (PS, Rp or Sp isomer) linkages provide protection against 3′ and/or 5′ exonucleases, increase protein binding, improve PK; chirality selection enhances RISC loading and metabolic stability, and are useful in siRNA conjugation.
5. Ribosugar modifications to increase stability: This design combines 2′-O-methyl(2′-OMe) and 2′-deoxy-2′-fluoro (2′-F) ribosugar modifications throughout both strands of the siRNA; this is associated with reduced immune stimulation.
6. 5′-block to increase efficacy: Modified nucleosides and analogs are provided that are useful for incorporation at one of the terminal positions of an oligomeric compound, e.g. the 5′-end of SS (sense strand). The strand with its 5′ terminus at the thermodynamically less stable end of the duplex is selected by RISC as the antisense strand. With creative design, preferential loading of the intended antisense strand can be achieved in most cases. However, loading of the sense strand into RISC cannot be excluded, especially when the thermodynamic asymmetry between the two ends of siRNAs is not significant. The presence of the monophosphate group at the 5′ end helps anchor the antisense strand in RISC, and there is an interaction between the 5′ monophosphate of the antisense strand and MID domain of the Argonaute 2—the protein component of RISC responsible for target cleavage. Hence, loading of the sense strand into the RISC could be impeded by blocking 5′ phosphorylation.

In some embodiments, the seed region, e.g., position 2-8, e.g., position 5, of the antisense strand (AS) (from 5′-end) is modified with one or more of GNA-S, GNA-R, UNA, and/or SNA. In some embodiments, the one or more modification reduces full-length on-target activity while mitigating off-target effect(s), for example, in a dual luciferase reporter gene assay. For example, in one embodiment, modifications with GNA-S and GNA-R at position 7 maintain on-target activity, while UNA and SNA modifications reduce full-length on-target activity but mitigate off-target effect(s), e.g., in a dual luciferase reporter gene assay.

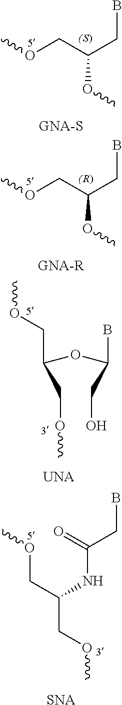

In certain embodiments, off-target effects of dsRNA molecules can be reduced or inhibited by incorporating thermally destabilizing nucleotides, e.g., GNA-S, GNA-R, UNA, and/or SNA, at selected positions in the antisense strand of the dsRNA, and optionally in the sense strand, e.g., as described in WO2018098328, the contents of which are incorporated herein by reference, in addition to or as an alternative to incorporating one or more novel nucleotide analogues as described herein.

In some embodiments, seed region modifications (nucleotides 2-8) on AS (antisense strand) may include, or be modified to include, one or more of the following nucleosides:

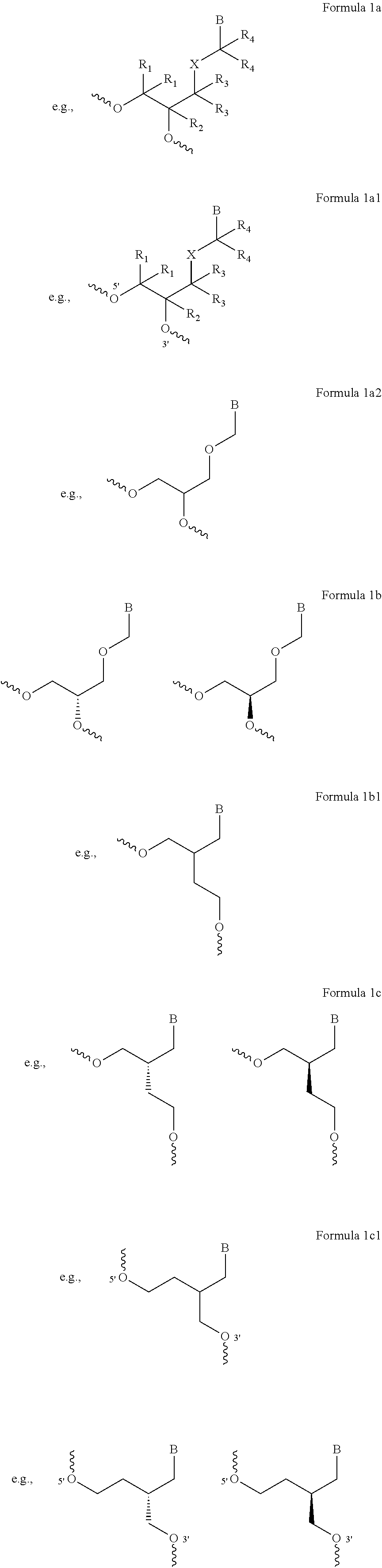

wherein
X is O, S, NH, —CONH—, or —NHCO—;
the wavy line represents attachment to the phosphate backbone of the oligonucleotide;
B is a modified or unmodified nucleobase;
R₁, R₂, R₃, and R₄ are independently H, halogen, —OR₅, or alkyl; and
R₅ is H, alkyl, cycloalkl, aryl, aralkyl, heteroaryl or sugar.

In any of the formulae described herein, if present and not defined, p, q, n and m are each independently, an integer is defined as follows: p=0-10, q=0-10, n=0-10, m=1-30.

For example, the disclosure provides nucleoside analogues of Formula 1a1 selected from Table A.

TABLE A

| Monomer | Corresponding code in antisense strand |
|---|---|
| [N3T12-027-01] | [N3T1-U] |
| [N3T12-031-01] | [N3T1-A] |
| [N3T12-068-01] | [N3T1-C] |
| [N3T12-069-01] | [N3T1-G] |
| [N3T12-027-01-R] | [N3T1-U-R] |
| [N3T12-027-01-S] | [N3T1-U-S] |
| [N3T12-031-01-R] | [N3T1-A-R] |
| [N3T12-031-01-S] | [N3T1-A-S] |
| [N3T12-047-01] | [N3T1-C-R] |
| [N3T12-070-01] | [N3T1-C-S] |
| [N3T12-048-01] | [N3T1-G-R] |
| [N3T12-071-01] | [N3T1-G-S] |
| [N3T12-072-01] | [N3T2-U] |
| [N3T12-073-01] | [N3T2-A] |
| [N3T12-074-01] | [N3T2-C] |
| [N3T12-075-01] | [N3T2-G] |
| [N3T12-072-01-S] | [N3T2-U-S] |
| [N3T12-072-01-R] | [N3T2-U-R] |
| [N3T12-073-01-S] | [N3T2-A-S] |
| [N3T12-073-01-R] | [N3T2-A-R] |
| [N3T12-074-01-S] | [N3T2-C-S] |
| [N3T12-074-01-R] | [N3T2-C-R] |
| [N3T12-075-01-S] | [N3T2-G-S] |
| [N3T12-075-01-R] | [N3T2-G-R] |

For example:

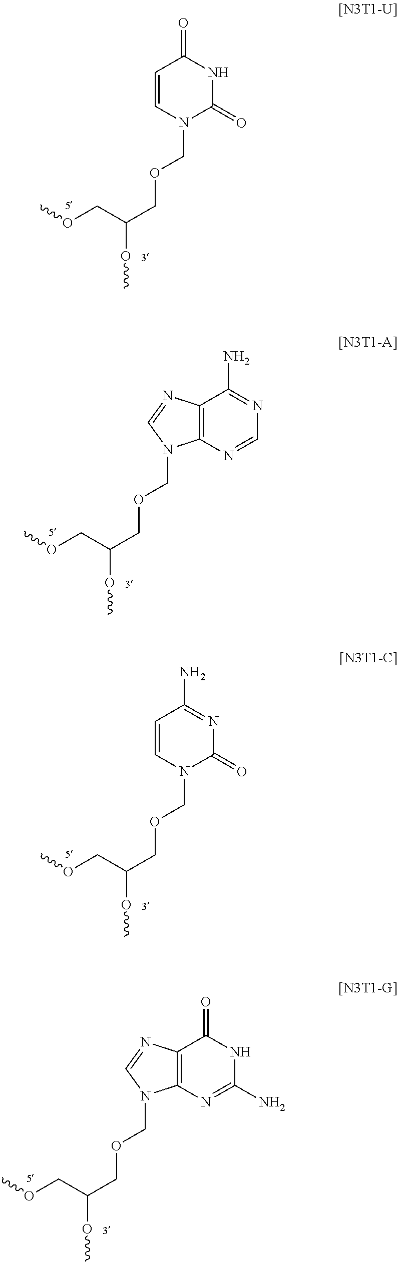

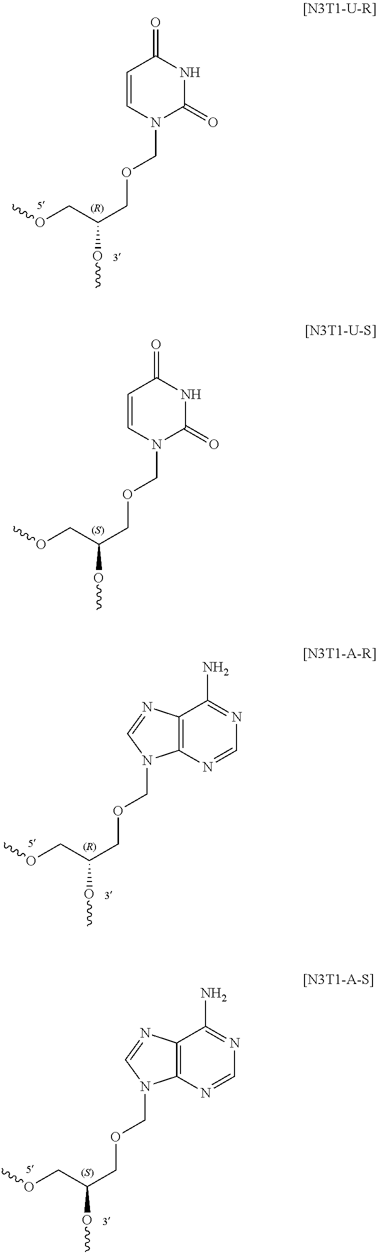

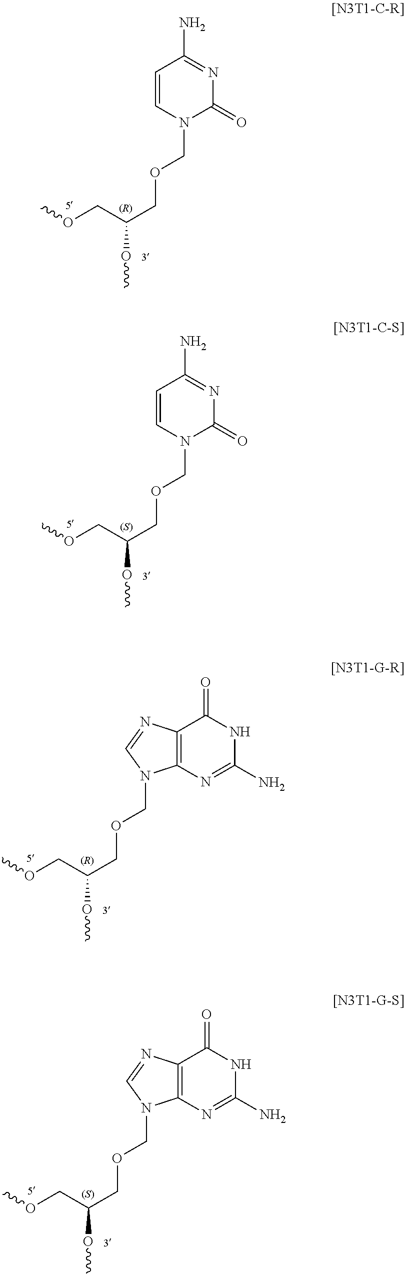

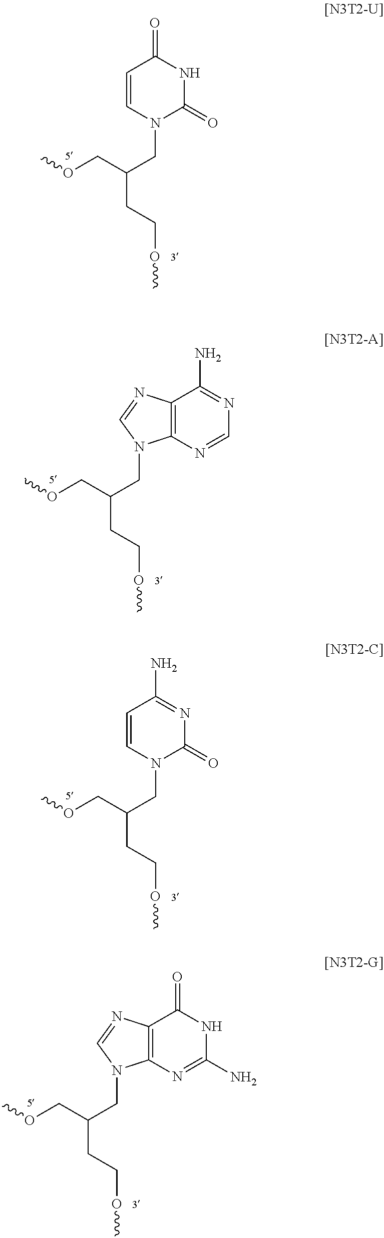

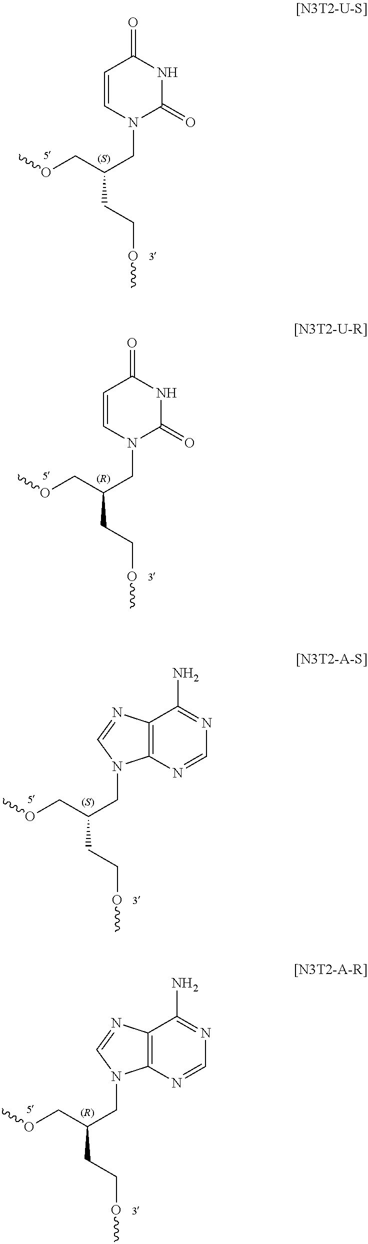

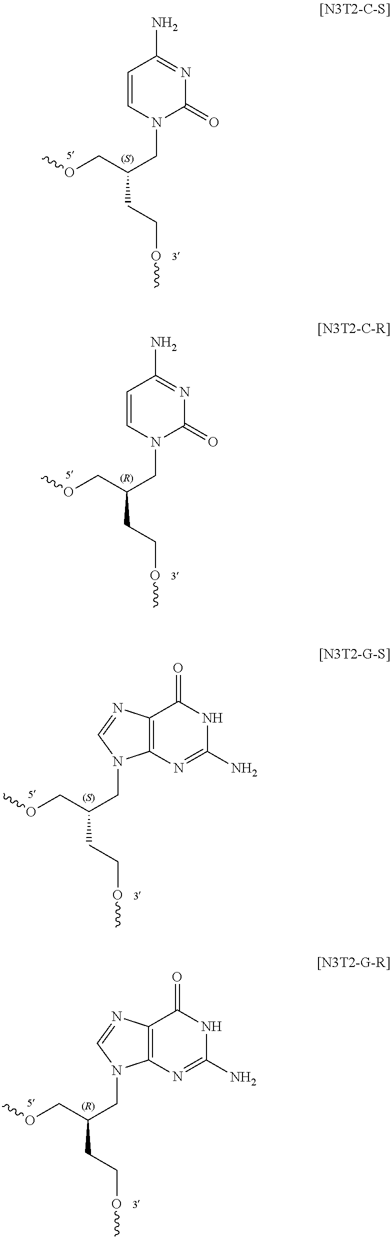

The disclosure thus provides a double-stranded siRNA duplex, comprising a sense strand sequence and a complementary antisense strand sequence, wherein the antisense sequence comprises one or more nucleoside analogues of Formula 1a or Formula 1b, e.g., wherein the antisense sequence comprises one or more nucleoside analogues selected from [N3T1-U], [N3T1-A], [N3T1-C], [N3T1-G], [N3T1-U—R], [N3T1-U—S], [N3T1-A-R], [N3T1-A-S], [N3T1-C—R], [N3T1-C—S], [N3T1-G-R], [N3T1-G-S], [N3T2-U], [N3T2-A], [N3T2-C], [N3T2-G], [N3T2-U—S], [N3T2-U—R], [N3T2-A-S], [N3T2-A-R], [N3T2-C—S], [N3T2-C—R], [N3T2-G-S], [N3T2-G-R]. The disclosure further provides a phosphoramidite intermediate of a nucleoside analogue of Formula 1a or Formula 1b, e.g., in O-protected form, for making a double-stranded siRNA duplex, wherein the antisense sequence comprises one or more nucleoside analogues of Formula 1a or Formula 1b. For example, the disclosure provides a nucleoside analogue selected from [N3T12-027-01], [N3T12-031-01], [N3T12-068-01], [N3T12-069-01], [N3T12-027-01-R], [N3T12-027-01-S], [N3T12-031-01-R], [N3T12-031-01-S], [N3T12-047-01], [N3T12-070-01], [N3T12-048-01], [N3T12-071-01], [N3T12-072-01], [N3T12-073-01], [N3T12-074-01], [N3T12-075-01], [N3T12-072-01-S], [N3T12-072-01-R], [N3T12-073-01-S], [N3T12-073-01-R], [N3T12-074-01-S], [N3T12-074-01-R], [N3T12-075-01-S], [N3T12-075-01-R] in phosphoramidite form, e.g.,

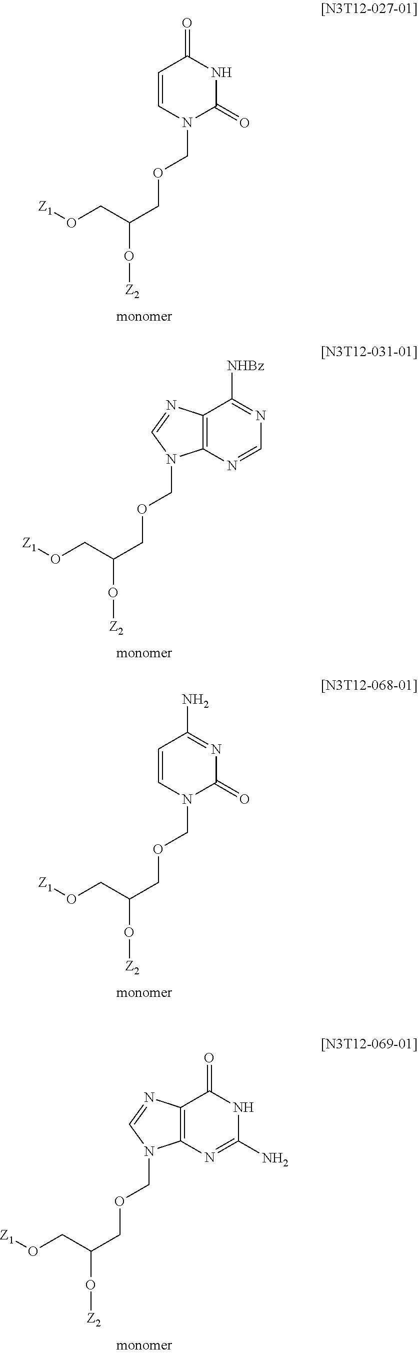

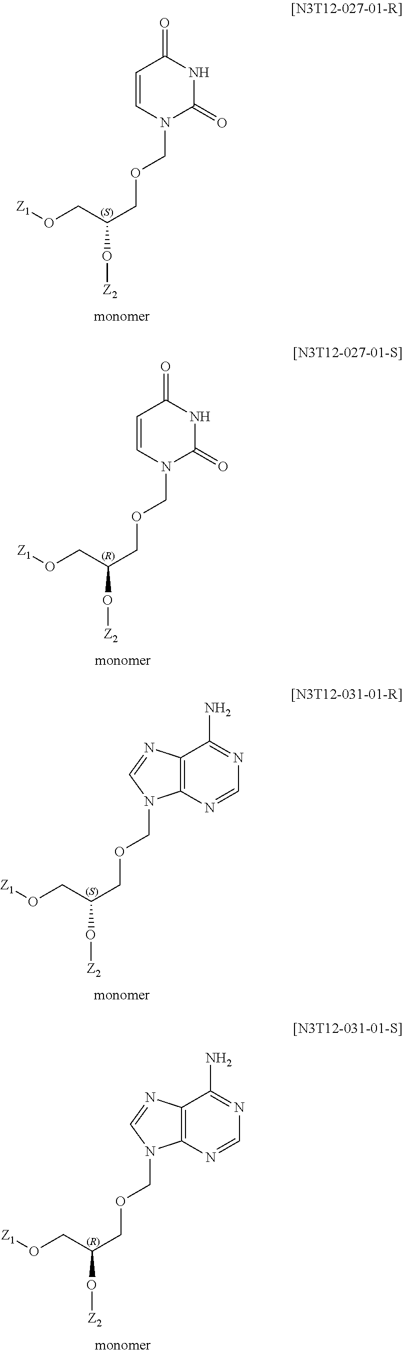

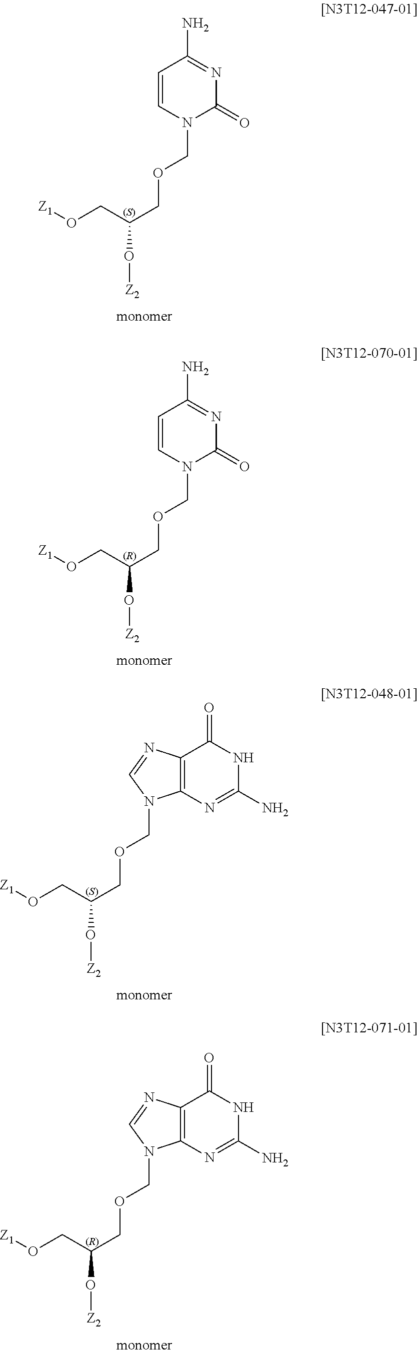

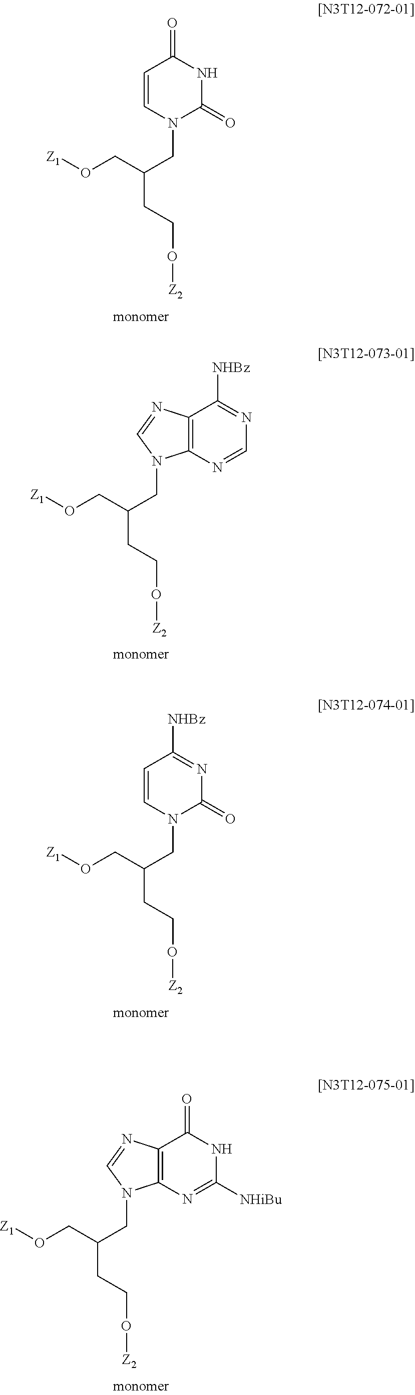

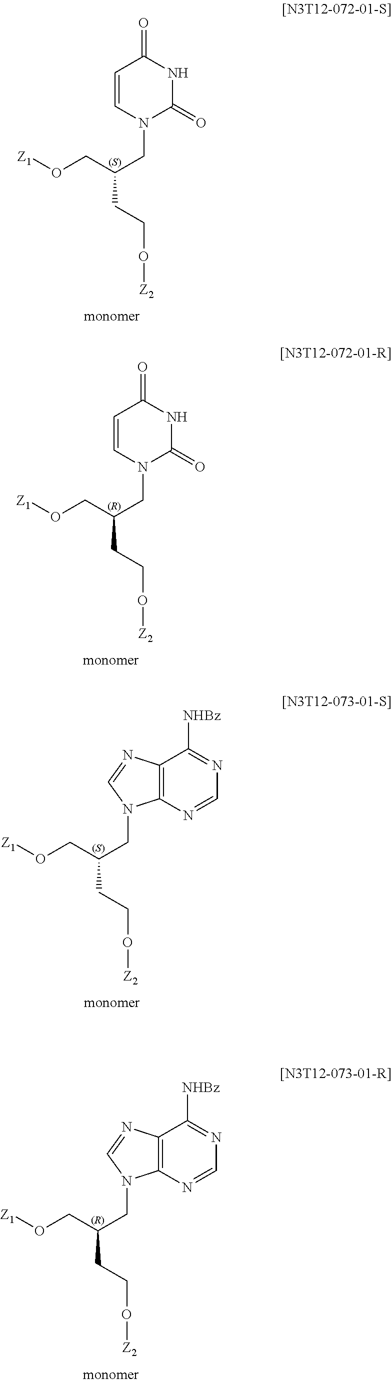

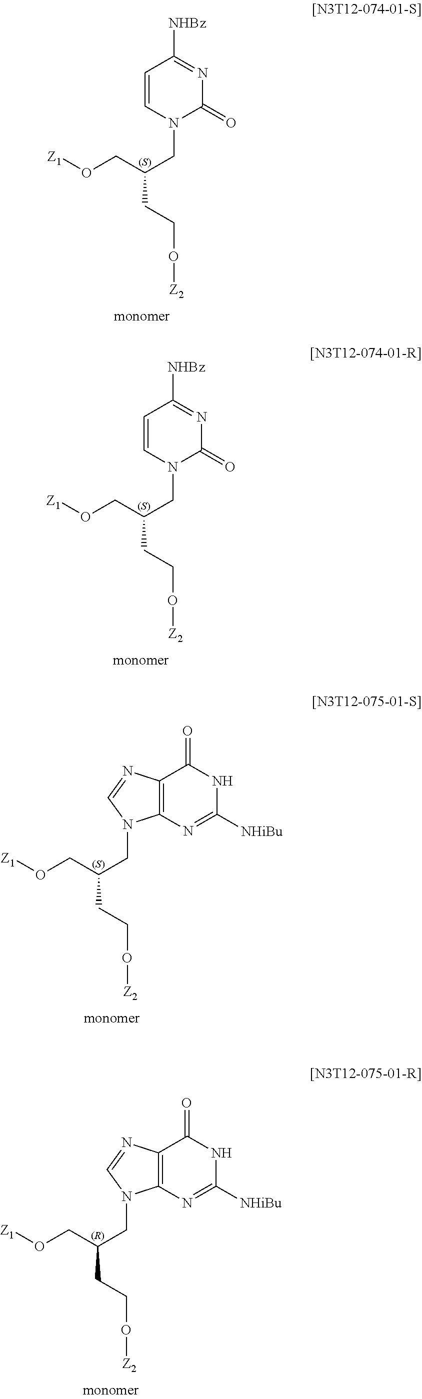

wherein
Z¹ is an O-protecting group, e.g., optionally substituted triphenylmethyl, e.g., dimethoxytrityl (DMTr);
Z² is a phosphoramidite group, e.g., [(2-cyanoethyl)-(N,N-diisopropyl)]-phosphoramidite; and
Y is H or an N-protecting group, e.g., benzyl or tert-butyloxycarbonyl (BOC). For example, the disclosure provides a nucleoside analogue selected from

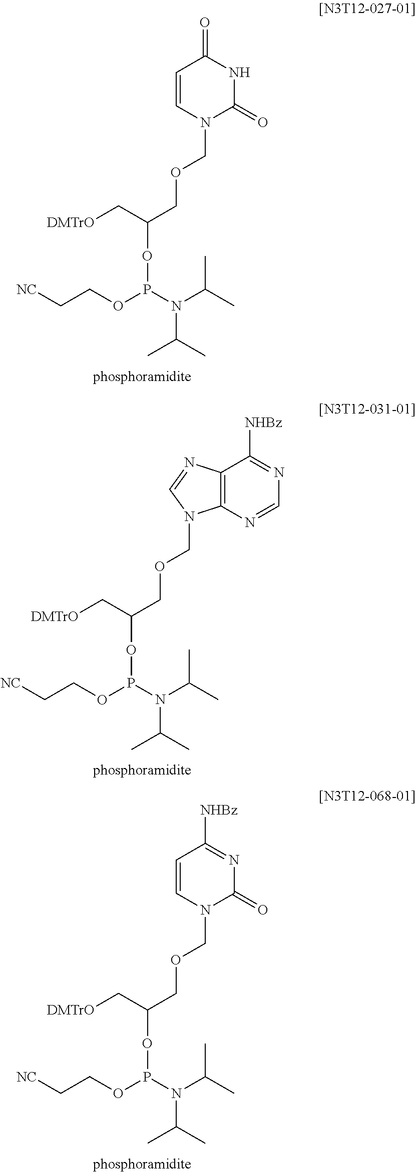

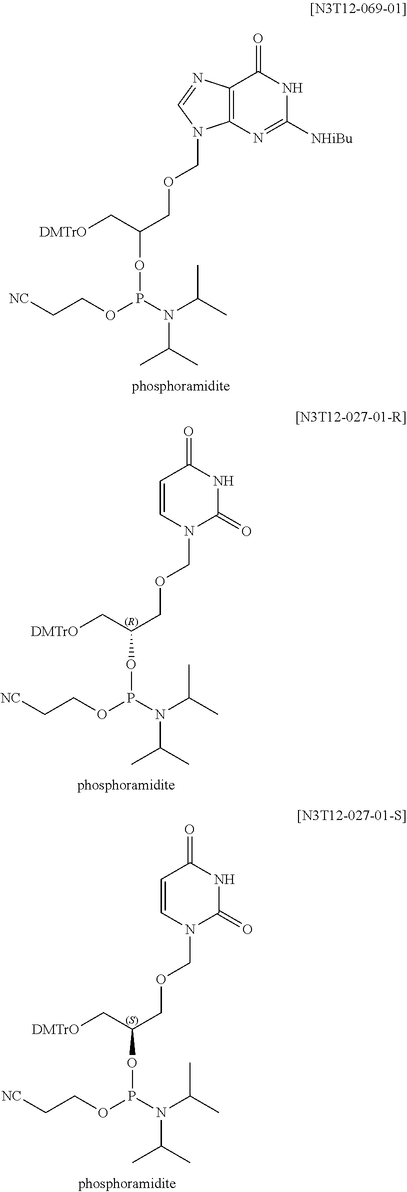

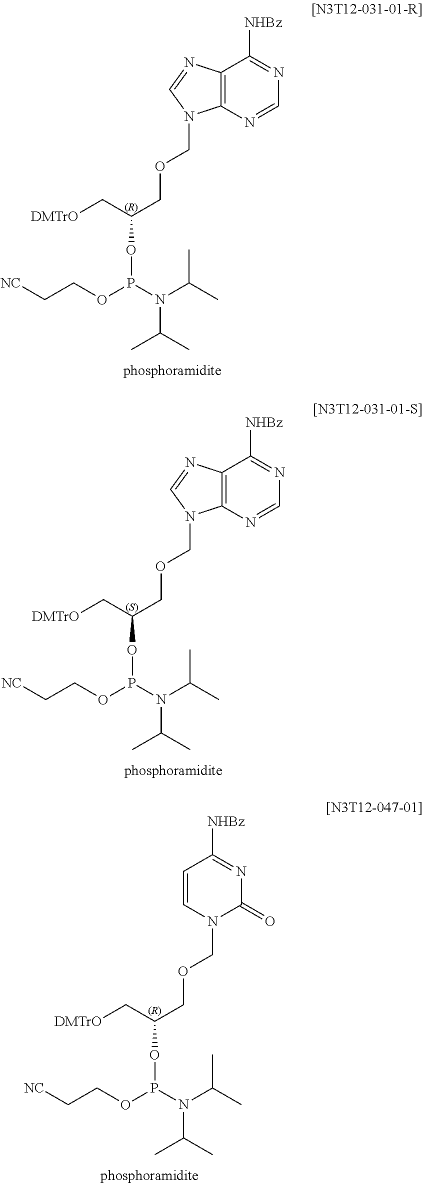

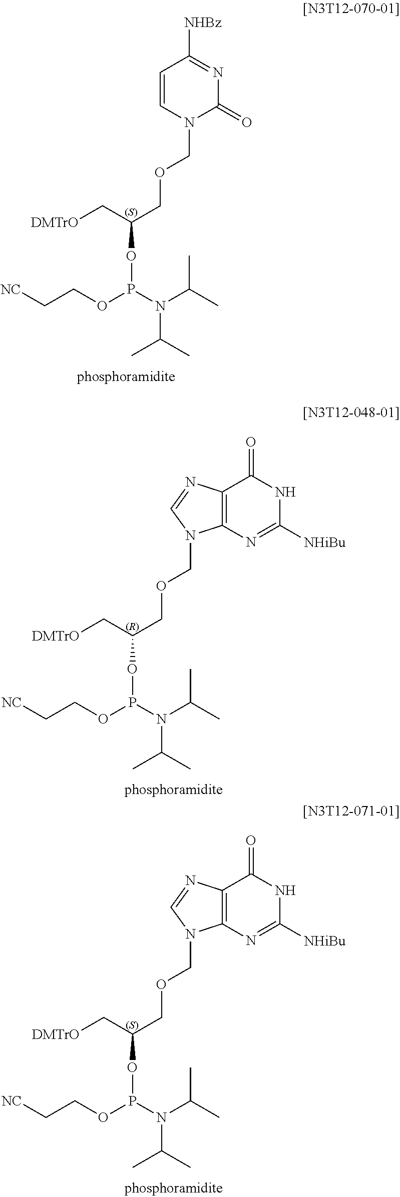

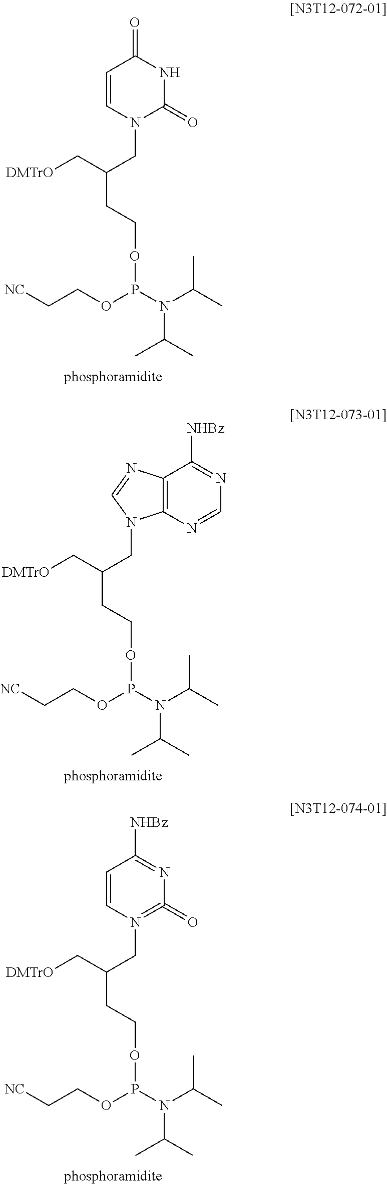

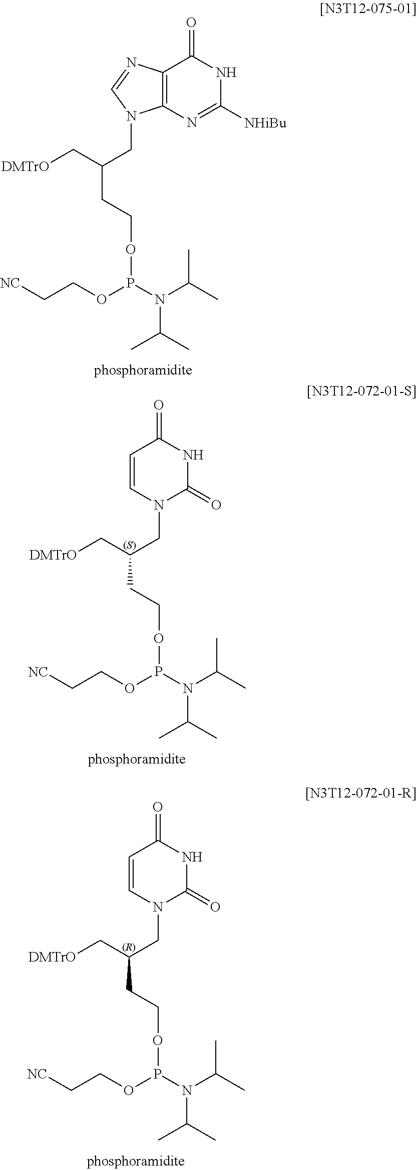

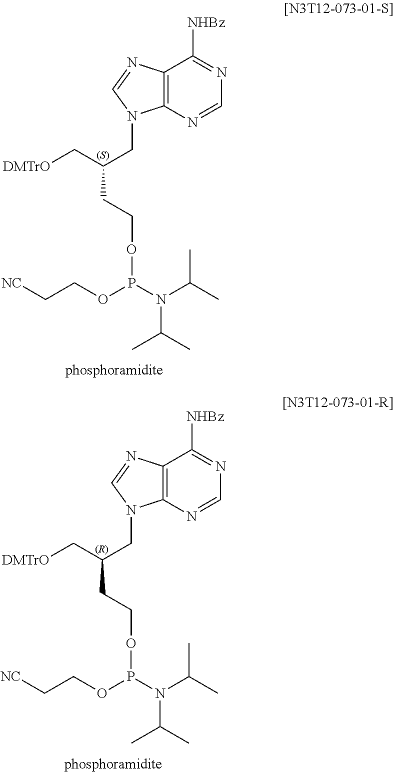

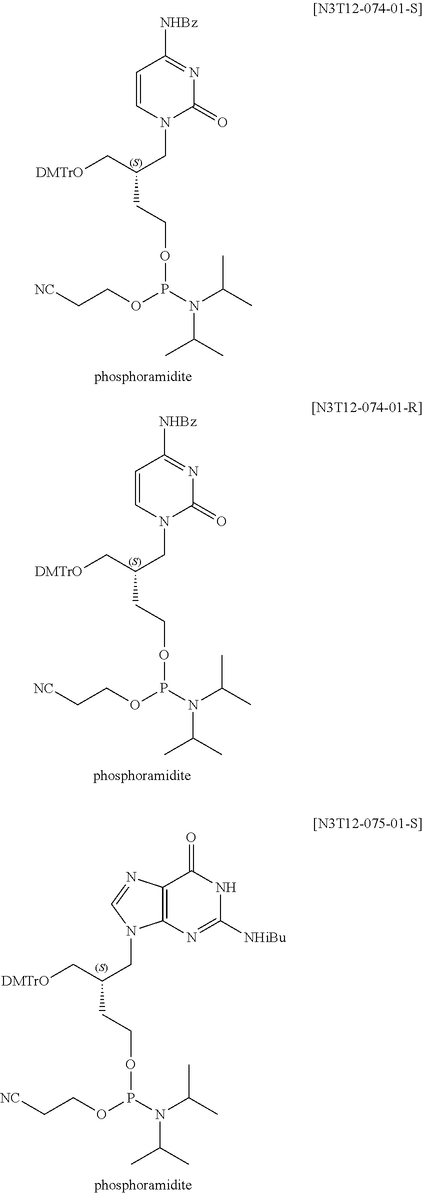

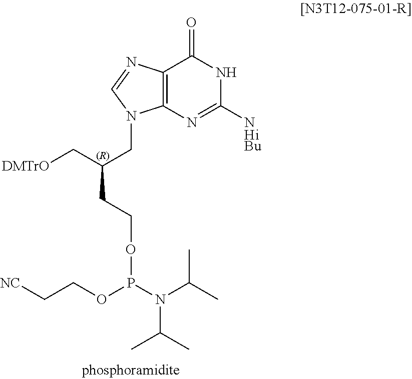

For example, the disclosure provides a method of making a double-stranded siRNA duplex, comprising a sense sequence and a complementary antisense sequence, wherein the antisense sequence comprises one or more nucleoside analogues of Formula 1a or Formula 1b, e.g., wherein the antisense sequence comprises one or more nucleoside analogues selected from [N3T12-027-01], [N3T12-031-01], [N3T1-U], [N3T1-A], [N3T1-C], [N3T1-G], [N3T1-U—R], [N3T1-U—S], [N3T1-A-R], [N3T1-A-S], [N3T1-C—R], [N3T1-C—S], [N3T1-G-R], [N3T1-G-S], [N3T2-U], [N3T2-A], [N3T2-C], [N3T2-G], [N3T2-U—S], [N3T2-U—R], [N3T2-A-S], [N3T2-A-R], [N3T2-C—S], [N3T2-C—R], [N3T2-G-S], and [N3T2-G-R].

In some embodiments, the disclosure provides modified siRNA which is an RNA duplex comprising a sense strand and an antisense strand, wherein the sense strand comprises one or more lipophilic moieties L₁ or L₂ conjugated to the siRNA via a phosphodiester or phosphorothioate bond accordance with Formula 2, below.

In some embodiments, the disclosure provides modified antisense oligonucleotide (ASO) wherein the modified antisense oligonucleotide (ASO) comprises one or more lipophilic moieties L₁ or L₂ conjugated to the ASO via a phosphodiester or phosphorothioate bond in accordance with Formula 2:

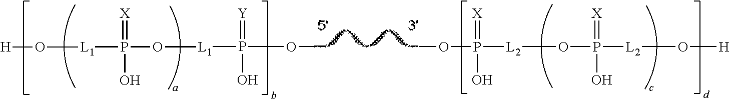

wherein
X═O or S;
a=0-4, e.g. 0-2;
b=0-4, e.g. 0-1;
c=0-4;
d=0-4;
provided that b and d are not both 0; and the ribbon represents the sense strand of the siRNA or antisense oligonucleotide (ASO).

In some embodiments,

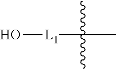

of the sense strand of siRNA or antisense oligonucleotide (ASO) is in accordance with any one of the following formulae:

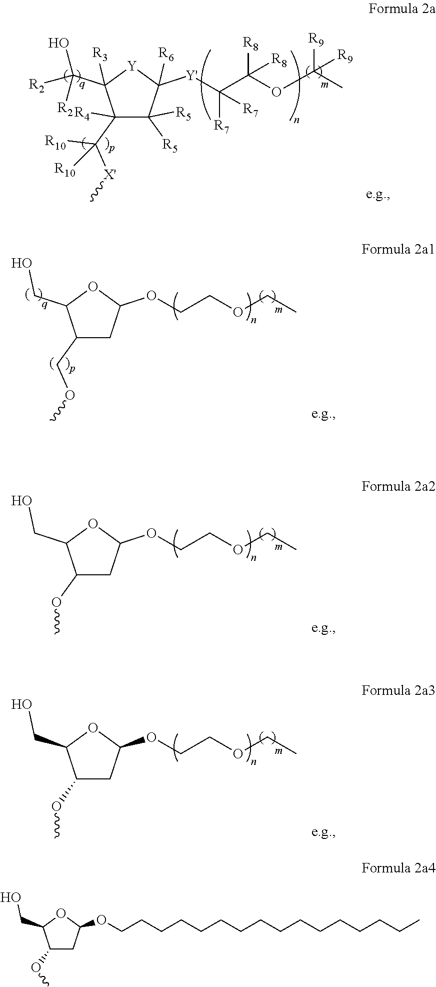

wherein,
R₂, R₃, R₄, R₅, R₆, R₇, R₈, R₉, R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, e.g. 0-2;
q=0-10, e.g. 1-2;
n=0-10;
m=1-30, e.g., 11-21, e.g. 13-17; e.g. 15.

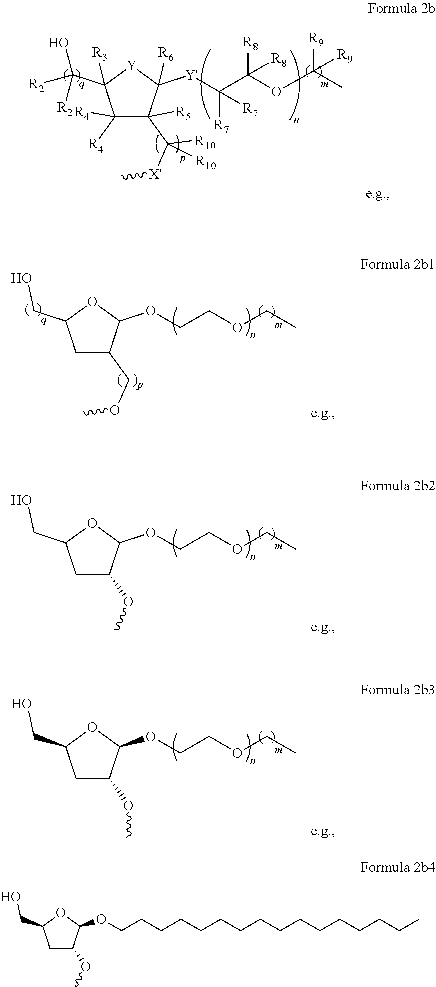

R₂, R₃, R₄, R₅, R₆, R₇, R₈, R₉, R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, e.g. 0-2;
q=0-10, e.g. 1-2;
n=0-10;
m=1-30, e.g., 11-21, e.g. 13-17; e.g. 15.

In some embodiments,

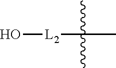

of the sense strand of siRNA or antisense oligonucleotide (ASO) is in accordance with any one of the following formulae:

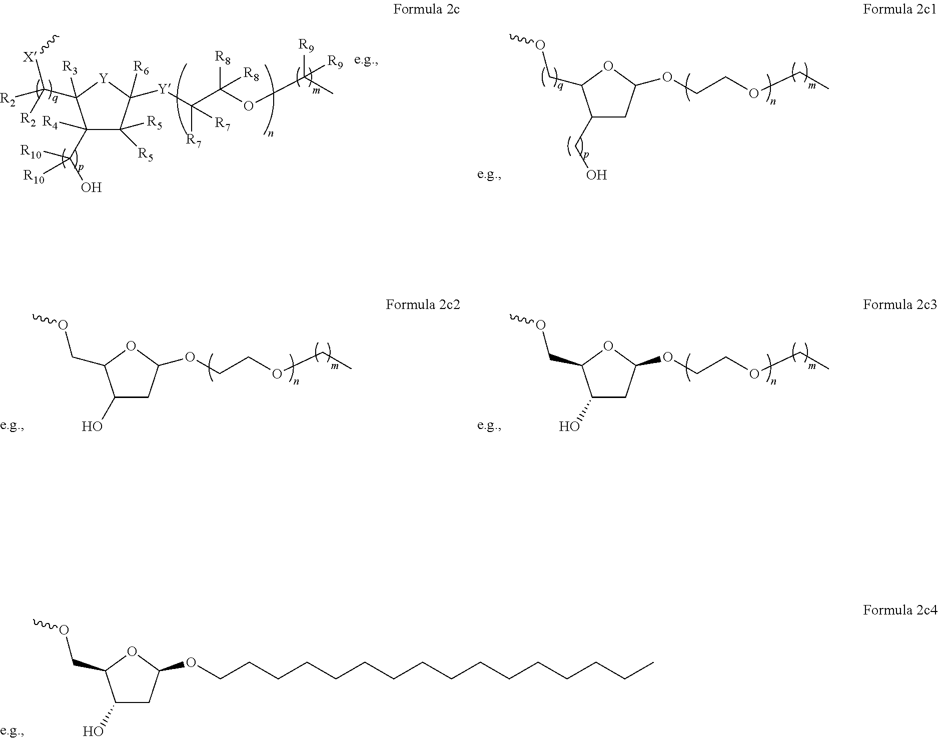

wherein,
R₂, R₃, R₄, R₅, R₆, R₇, R₈, R₉, R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, e.g. 0-2;
q=0-10, e.g. 1-2;
n=0-10;
m=1-30, e.g., 11-21, e.g. 13-17; e.g. 15.;

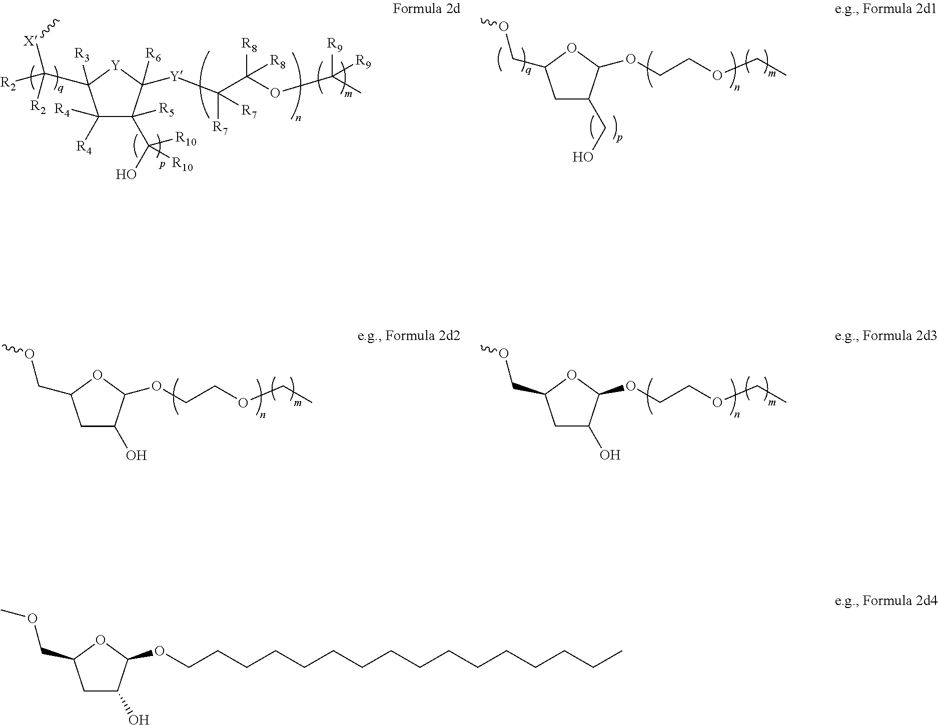

wherein,
R₂, R₃, R₄, R₅, R₆, R₇, R₈, R₉, R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, e.g. 0-2;
q=0-10, e.g. 1-2;
n=0-10;
m=1-30, e.g., 11-21, e.g. 13-17; e.g. 15.

In some embodiments,

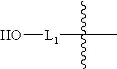

of the sense strand of siRNA or antisense oligonucleotide (ASO) is in accordance with any one of the following formulae:
Formula 2e:

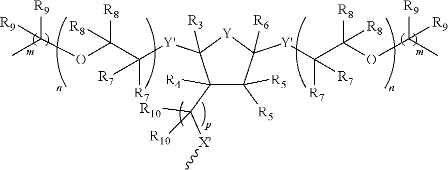

wherein a=0, b=1, c=0, and d=0;

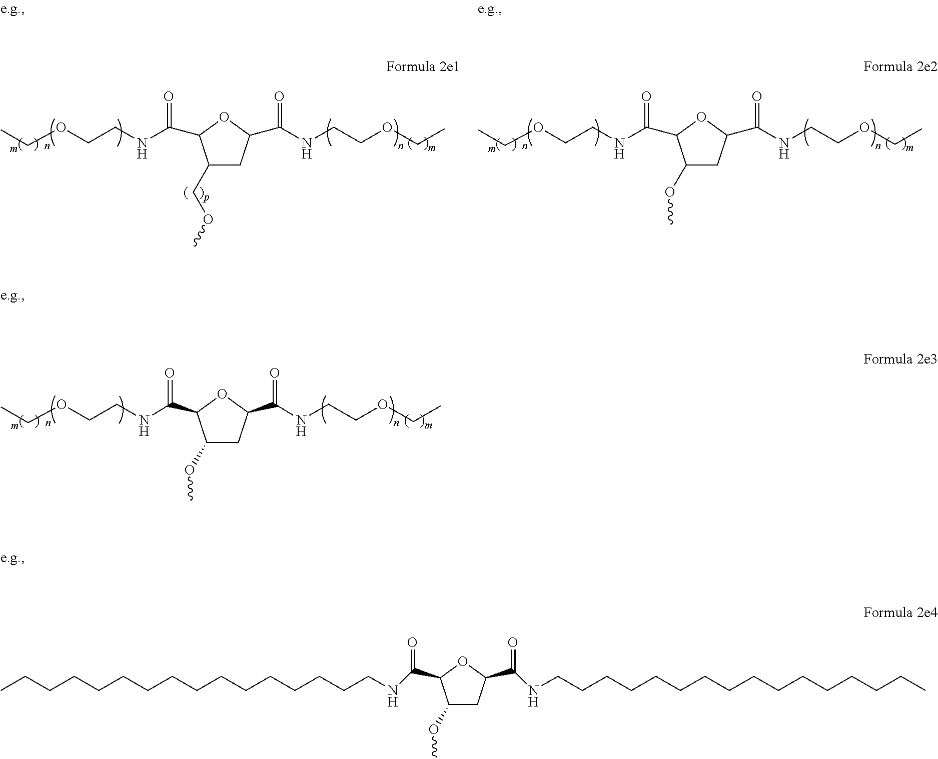

wherein,
R₃, R₄, R₅, R₆, R₇, R₈, R₉, R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, e.g. 0-2;
n=0-10;
m=1-30, e.g., 11-21, e.g. 13-17; e.g. 15.;

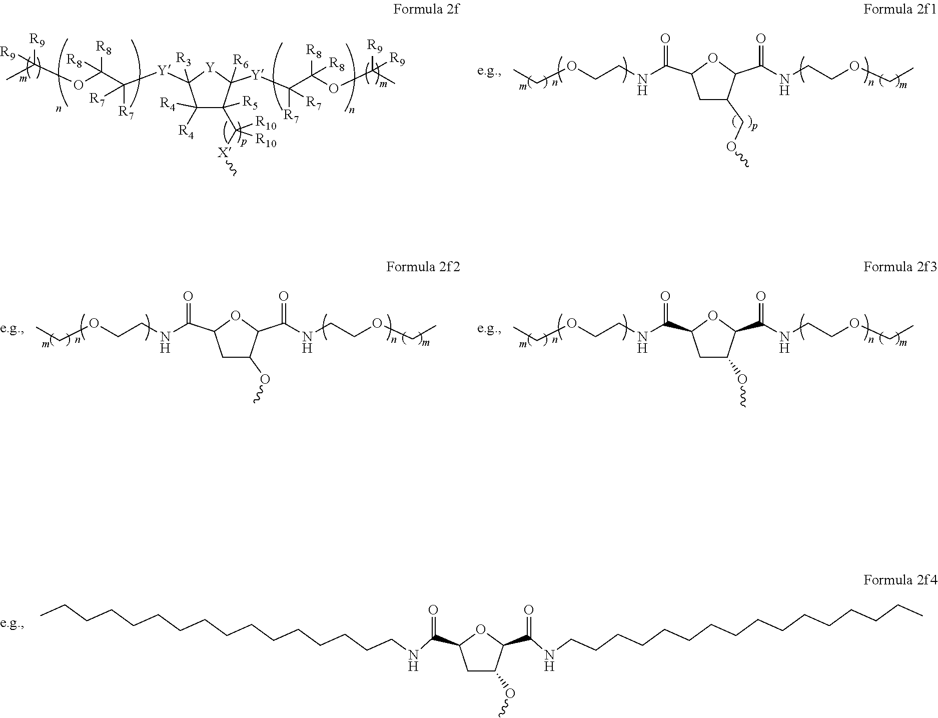

wherein,
R₃, R₄, R₅, R₆, R₇, Rs, R₉, R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, e.g. 0-2;
n=0-10;
m=1-30, e.g., 11-21, e.g. 13-17; e.g. 15.

In some embodiments,

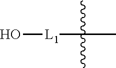

of the sense strand of siRNA or antisense oligonucleotide (ASO) is in accordance with any one of the following formulae:

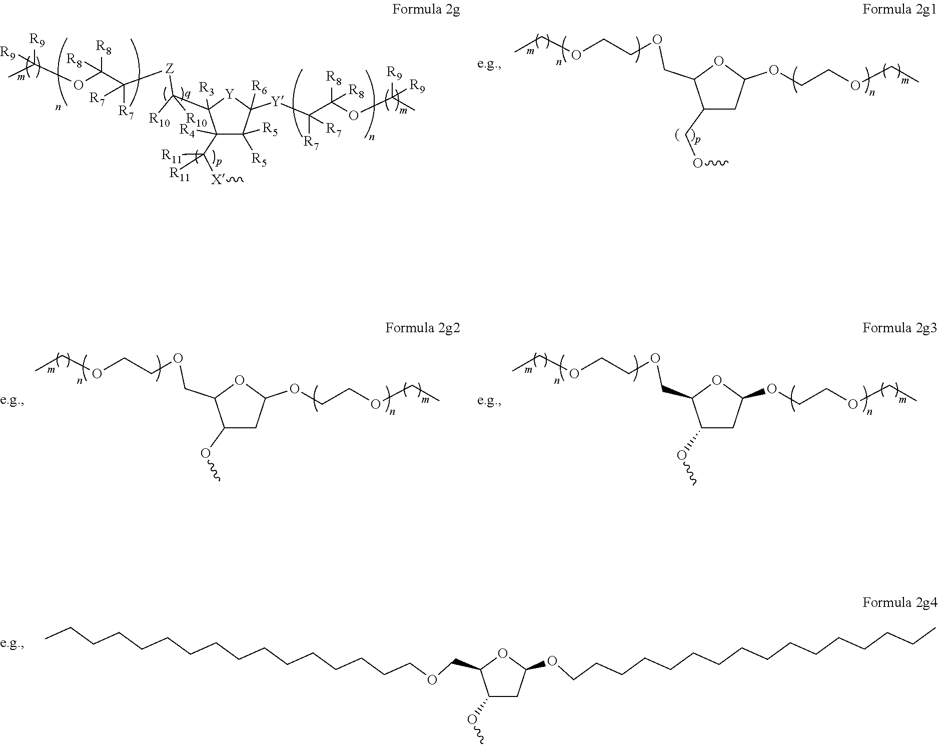

wherein,
R₂, R₃, R₄, R₅, R₆, R₇, Rs, R₉, R₁₀ and R₁₁ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, e.g. 0-2;
q=0-10, e.g. 1-2;
n=0-10;
m=1-30, e.g., 11-21, e.g. 13-17; e.g. 15.;

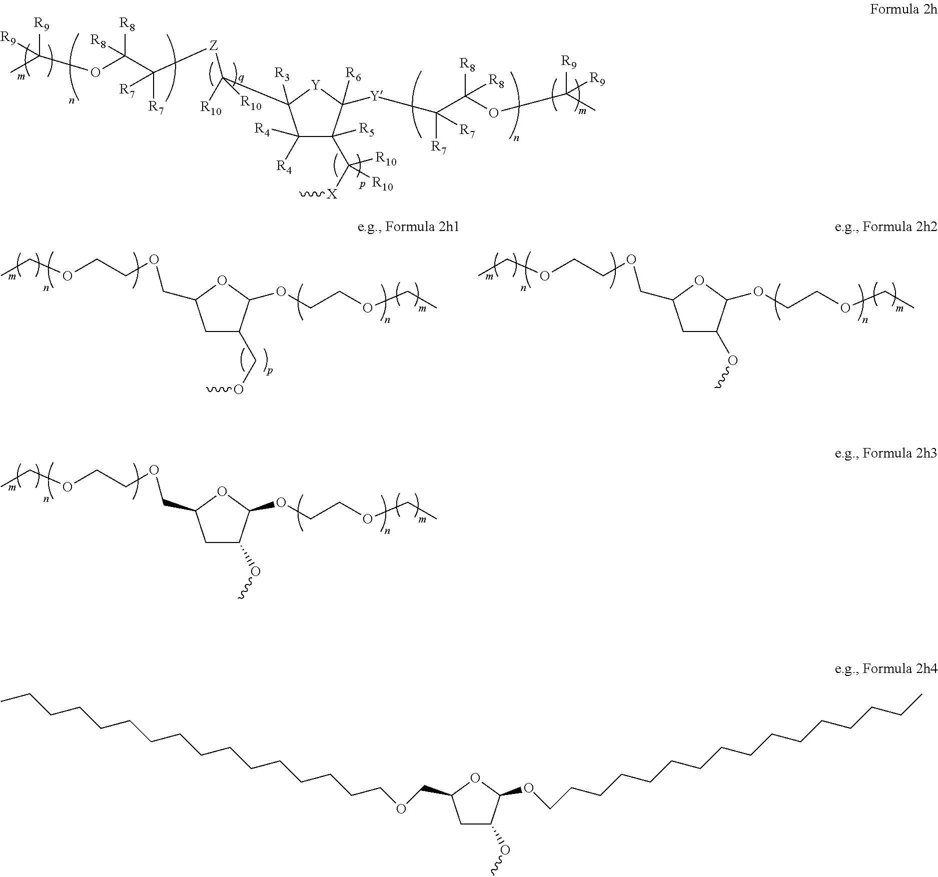

wherein,
R₃, R₄, R₅, R₆, R₇, R₈, R₉, R₁₀, and Rᵤ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, —CH₂O—, —OCH₂—, or —SS—;
Z is O, S, CH₂, NH, —NHCO—, —CONH—, or —SS—;
p=0-10, e.g. 0-2;
q=1-10, e.g. 1-2;
n=0-10;
m=1-30, e.g., 11-21, e.g. 13-17; e.g. 15.;

provided that the O with the wavy line on said L₁ or L₂ is part of a phosphodiester or phosphorothiate bond to the sense strand of siRNA or antisense oligonucleotide (ASO).

In some embodiments, modification moieties may be combined, as exemplified by the following structures:

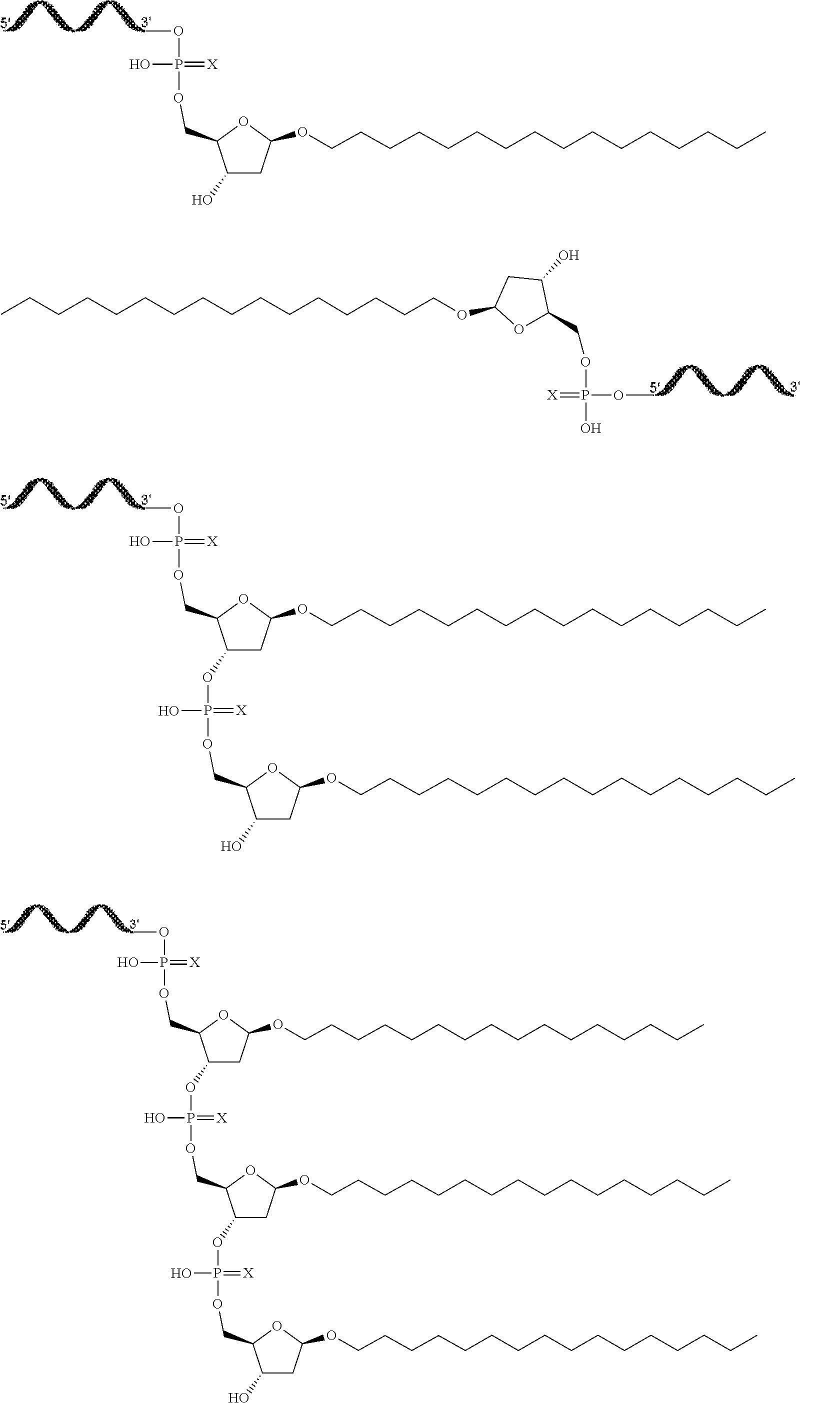

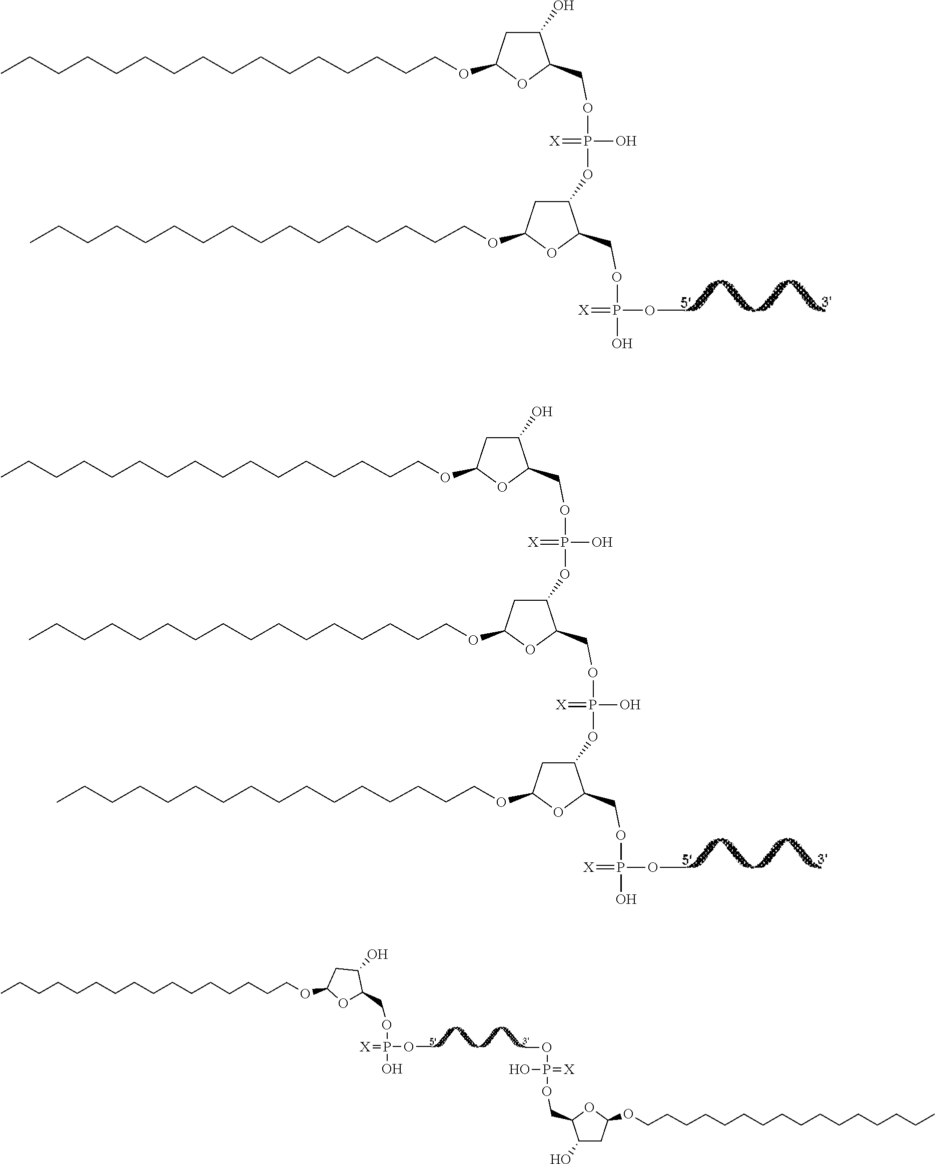

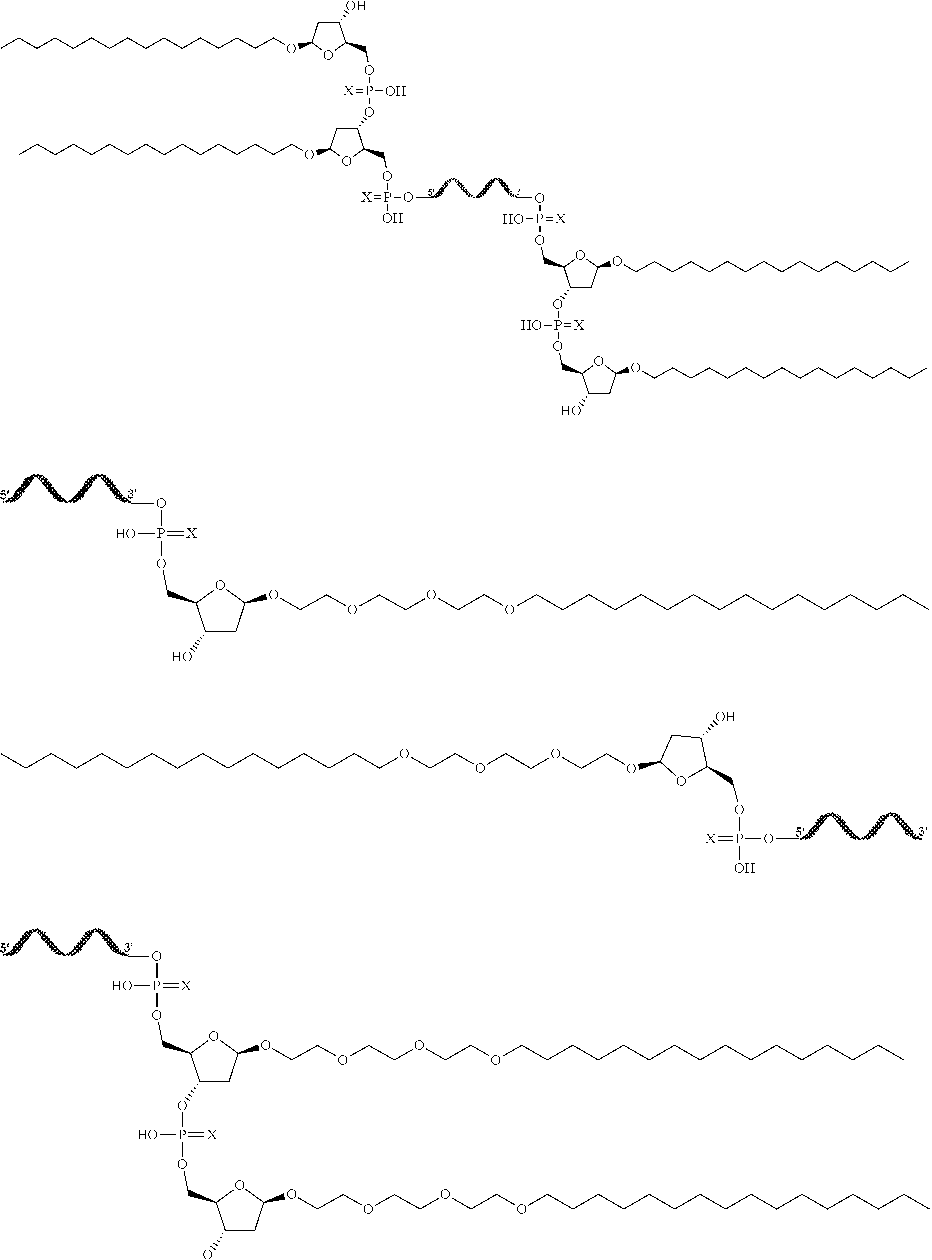

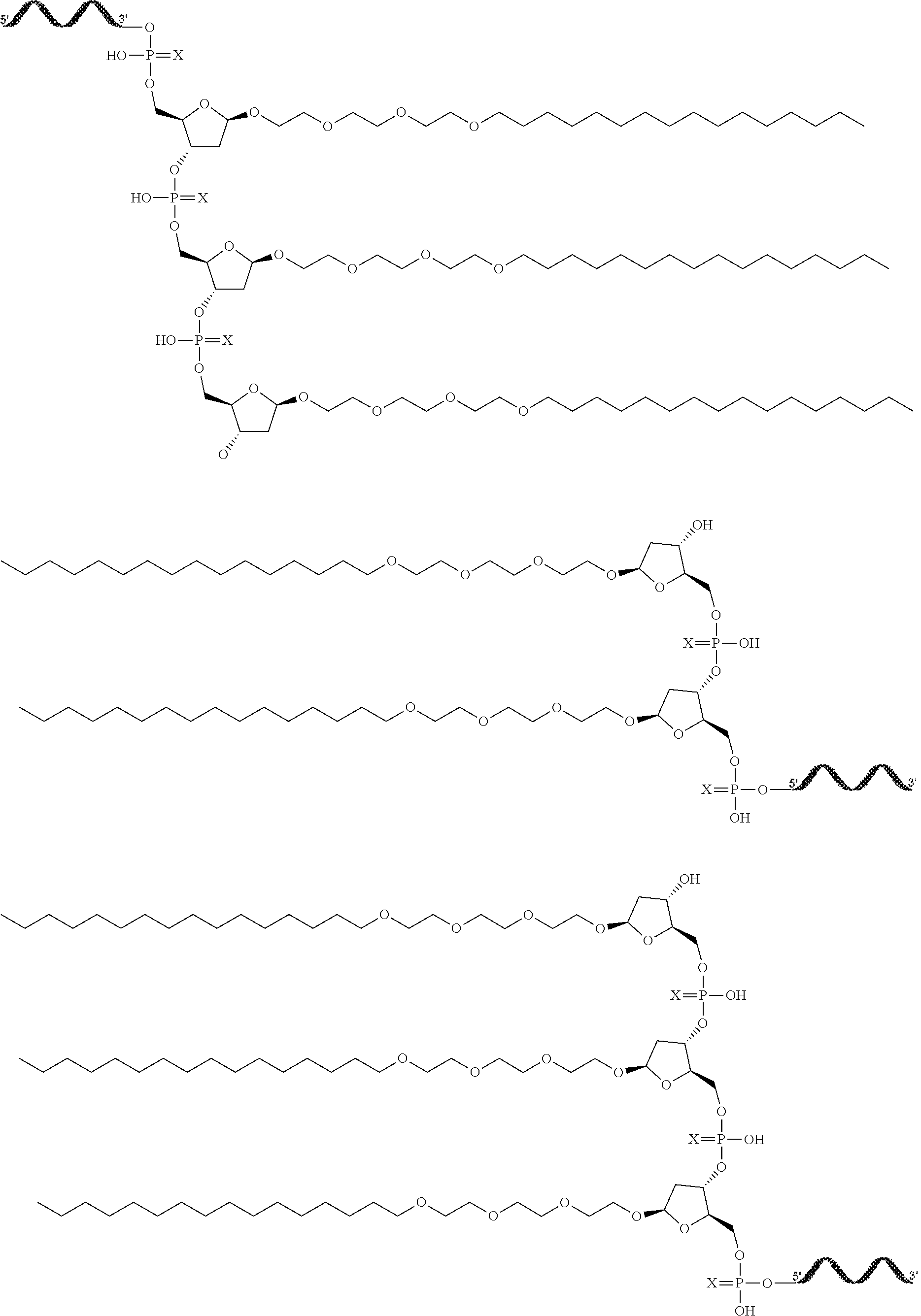

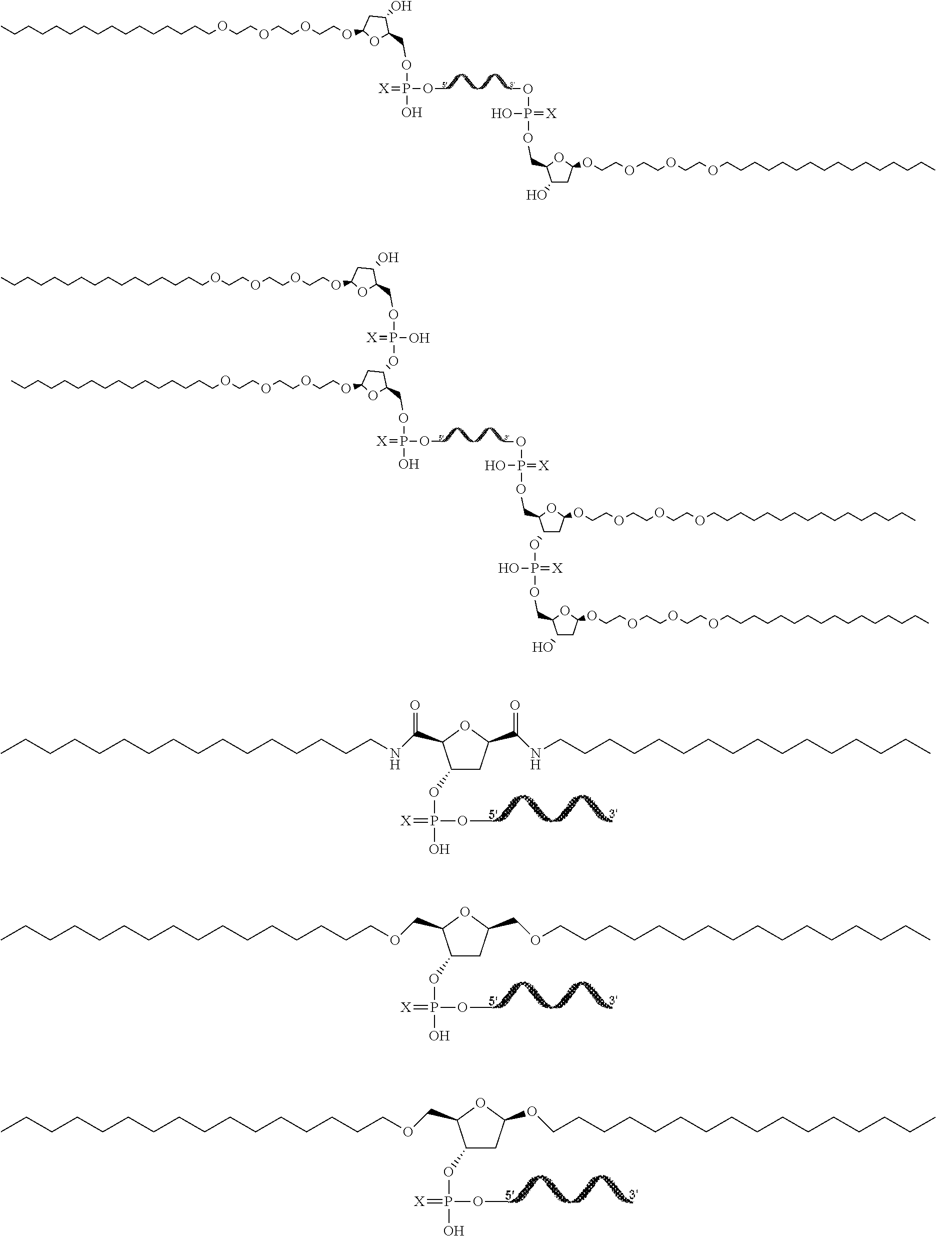

wherein
X═O or S; and the ribbon represents the sense strand of siRNA or antisense oligonucleotide (ASO).

In some embodiments, selected nucleotides are linked to 2′-O lipid conjugate moieties or adamantyl groups on sense strand of siRNA or antisense oligonucleotide (ASO):

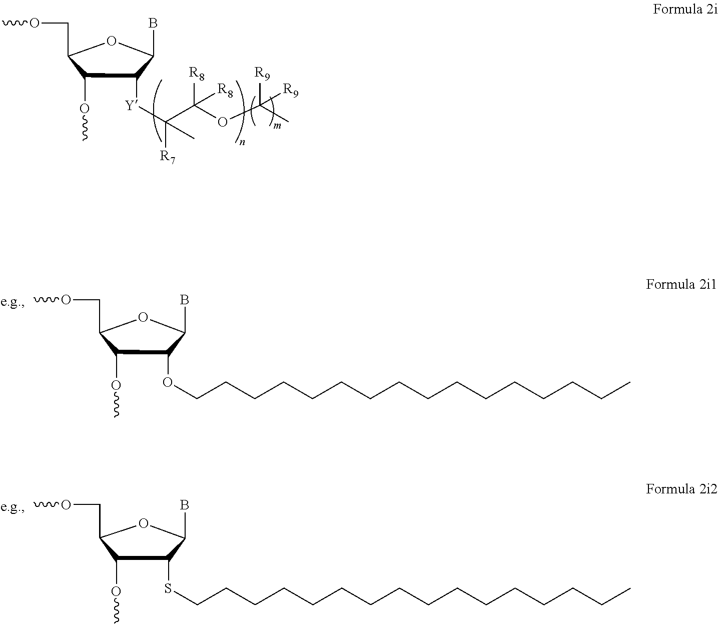

wherein,
R₇, Rs and R₉ are each independently H, halogen, or C1-C6 alkyl, optionally substituted with one or more halogen and/or hydroxy;
Y′ is O, S, CH₂, NH, —NHCO—, —CONH—, —CH₂O—, or —SS—;
n=0-10;
m=1-30, e.g., 11-21, e.g. 13-17; e.g. 15.

In some embodiments, selected nucleotides are linked to 5′O-lipid conjugate moieties or adamantyl groups on sense strand of siRNA or antisense oligonucleotide (ASO):

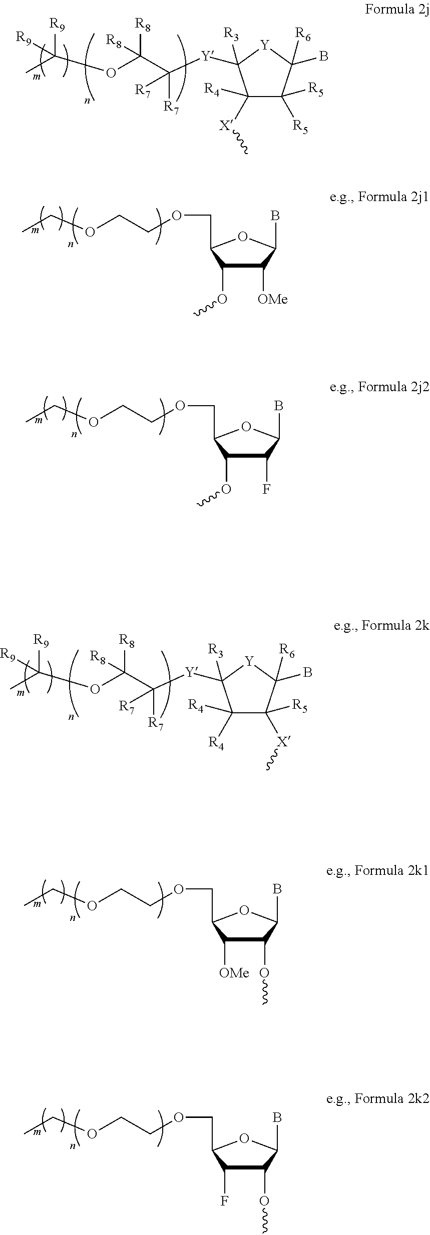

For example:

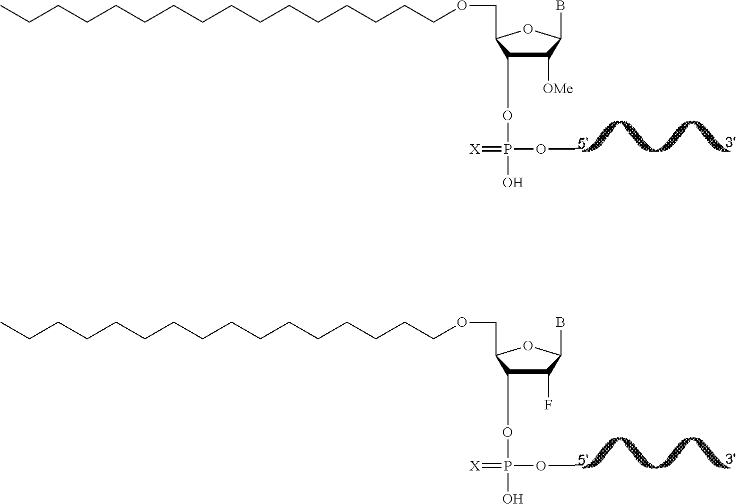

wherein B is a modified or unmodified nucleobase; and the ribbon represents the sense strand of siRNA or antisense oligonucleotide (ASO).
R₃, R₄, R₅, R₆, R₇, R₈, and R₉ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, —CH₂O—, —OCH₂—, or —SS—;
n=0-10;
m=1-30, e.g., 11-21, e.g. 13-17; e.g. 15.

In some embodiments,

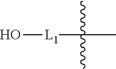

of the sense strand of siRNA or antisense oligonucleotide (ASO) is in accordance with any one of the following formulae:

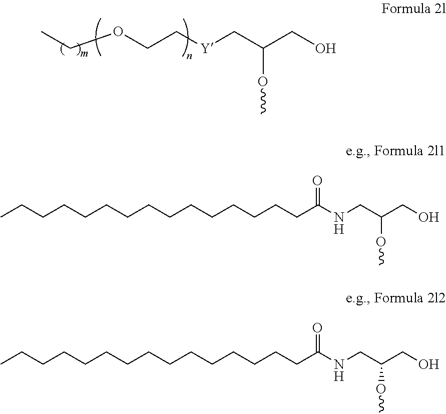

wherein,
Y′ is O, S, CH₂, NH, —NHCO—, —CONH—, or —SS—;
n=0-10;
m=1-30, e.g., 11-21, e.g. 13-17; e.g. 15.

In some embodiments,

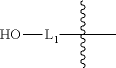

of the sense strand of siRNA or antisense oligonucleotide (ASO) is in accordance with any one of the following formulae:

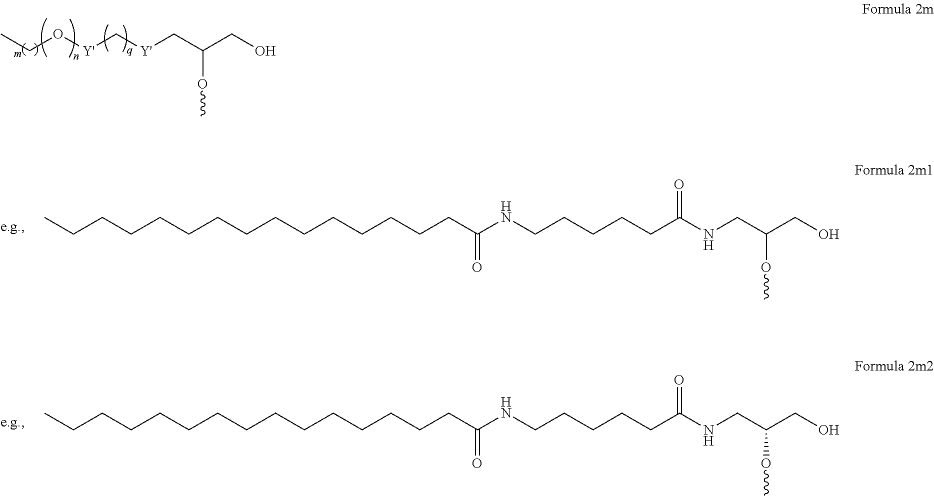

wherein,
Y′ is O, S, CH₂, NH, —NHCO—, —CONH—;
q=1-20;
n=0-10;
m=1-30, e.g., 11-21, e.g. 13-17; e.g. 15.

In some embodiments,

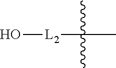

of the sense strand of siRNA or antisense oligonucleotide (ASO) is in accordance with any one of the following formulae:

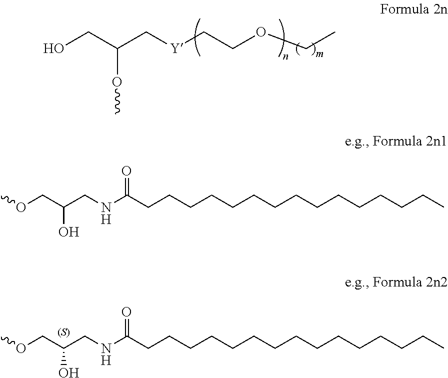

wherein,
Y′ is O, S, CH₂, NH, —NHCO—, —CONH—;
n=0-10;
m=1-30, e.g., 11-21, e.g. 13-17; e.g. 15.

In some embodiments,

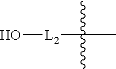

of the sense strand of siRNA or antisense oligonucleotide (ASO) is in accordance with any one of the following formulae:

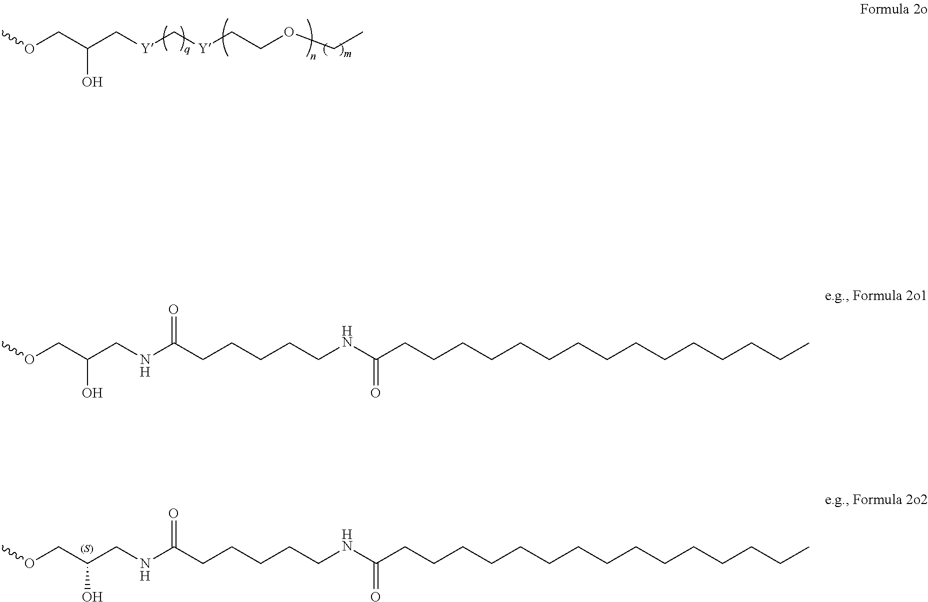

wherein,
Y′ is O, S, CH₂, NH, —NHCO—, —CONH—, or —SS—;
q=1-10;
n=0-10;
m=1-30, e.g., 11-21, e.g. 13-17; e.g. 15.

In some embodiments, exemplary structures include:

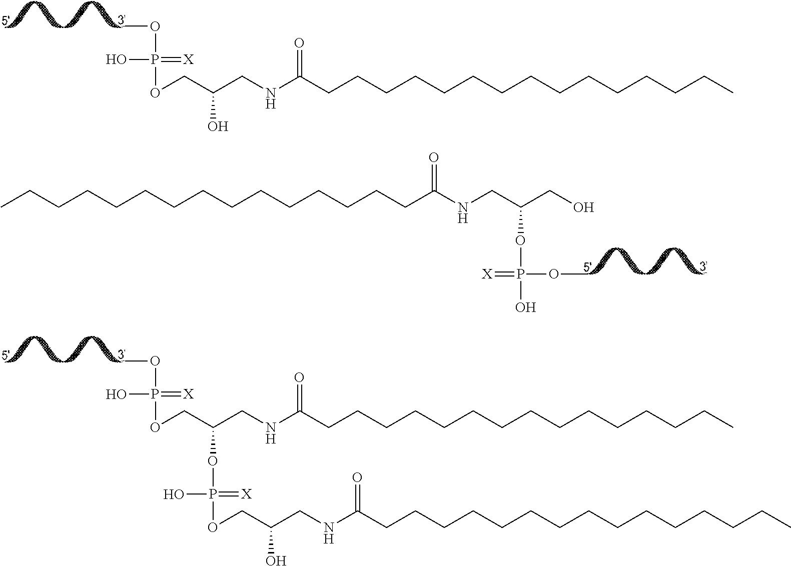

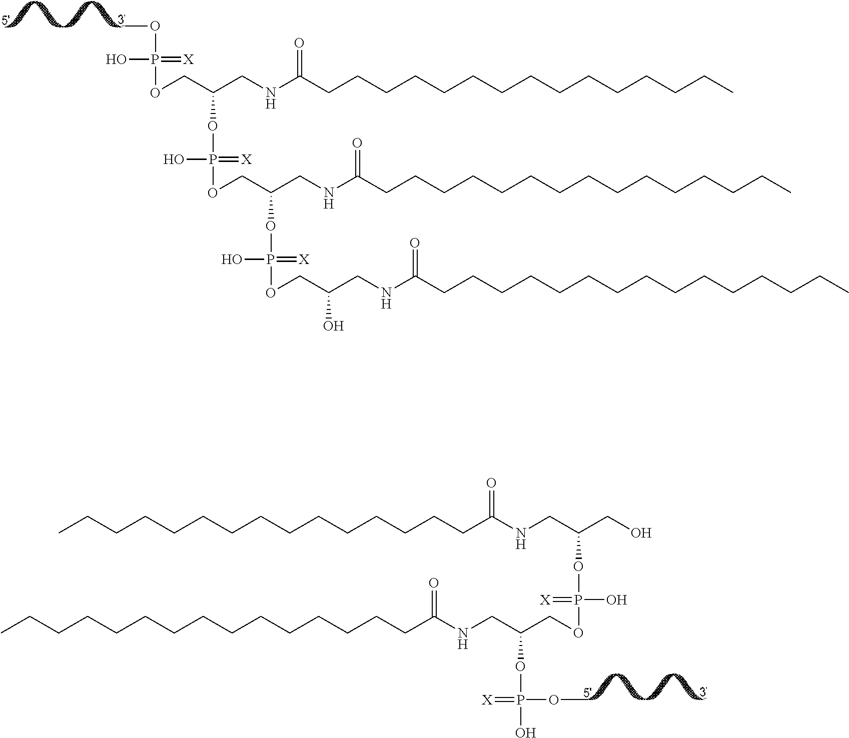

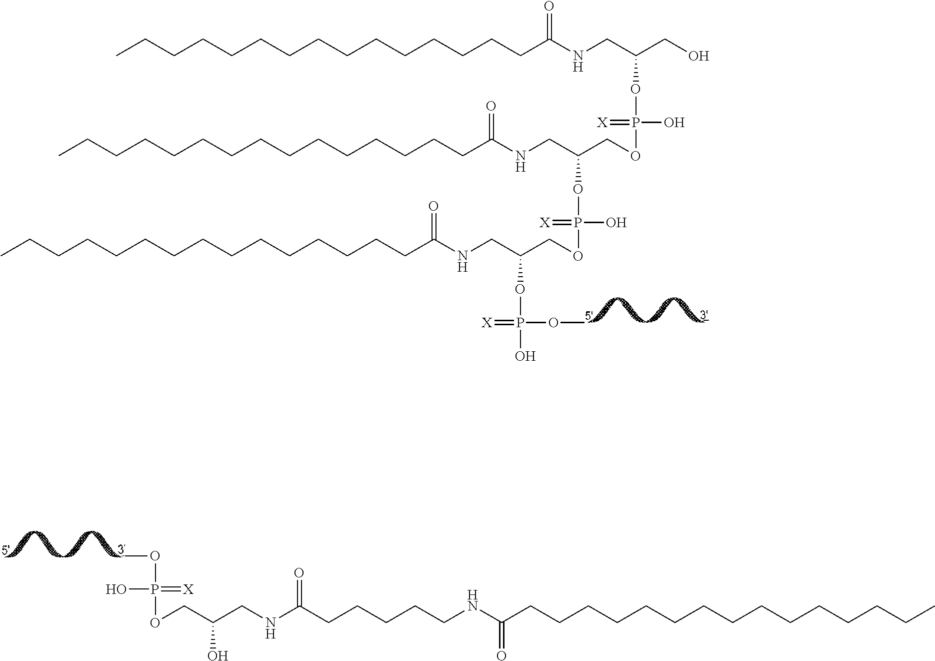

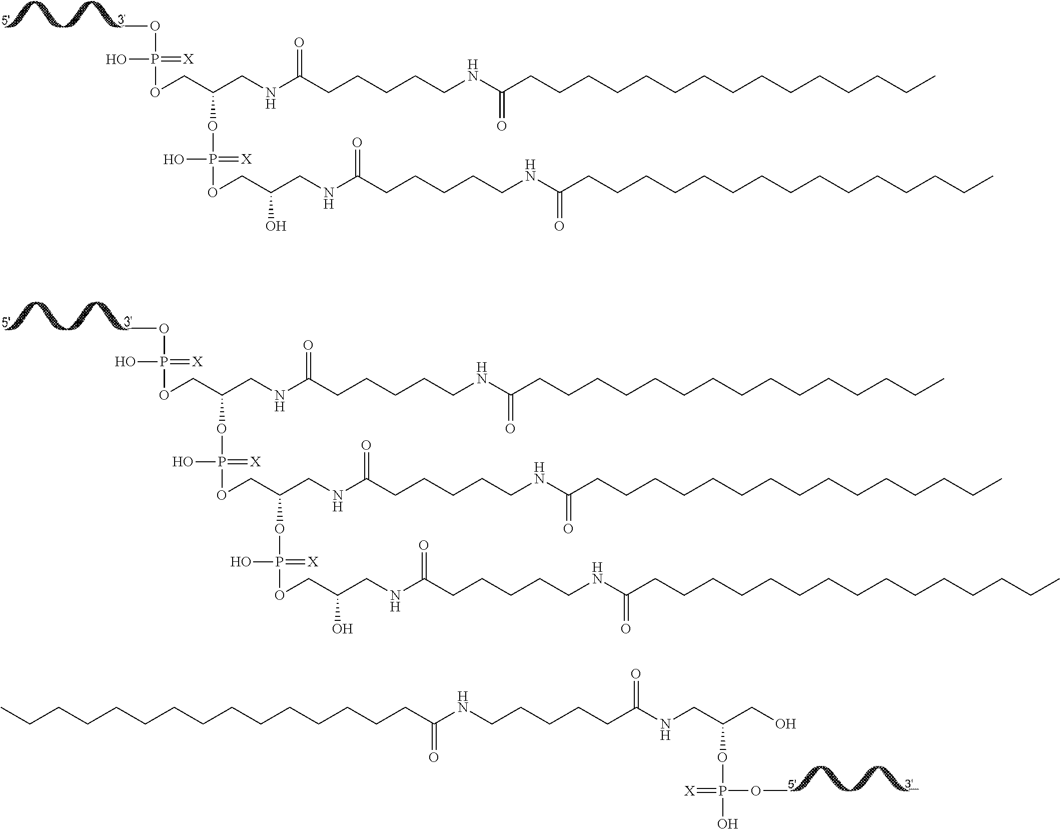

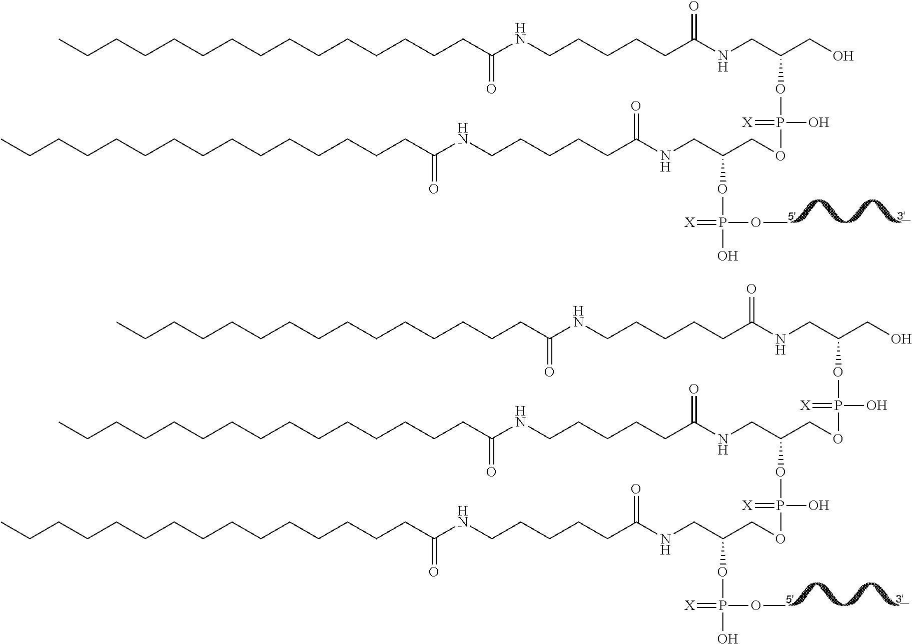

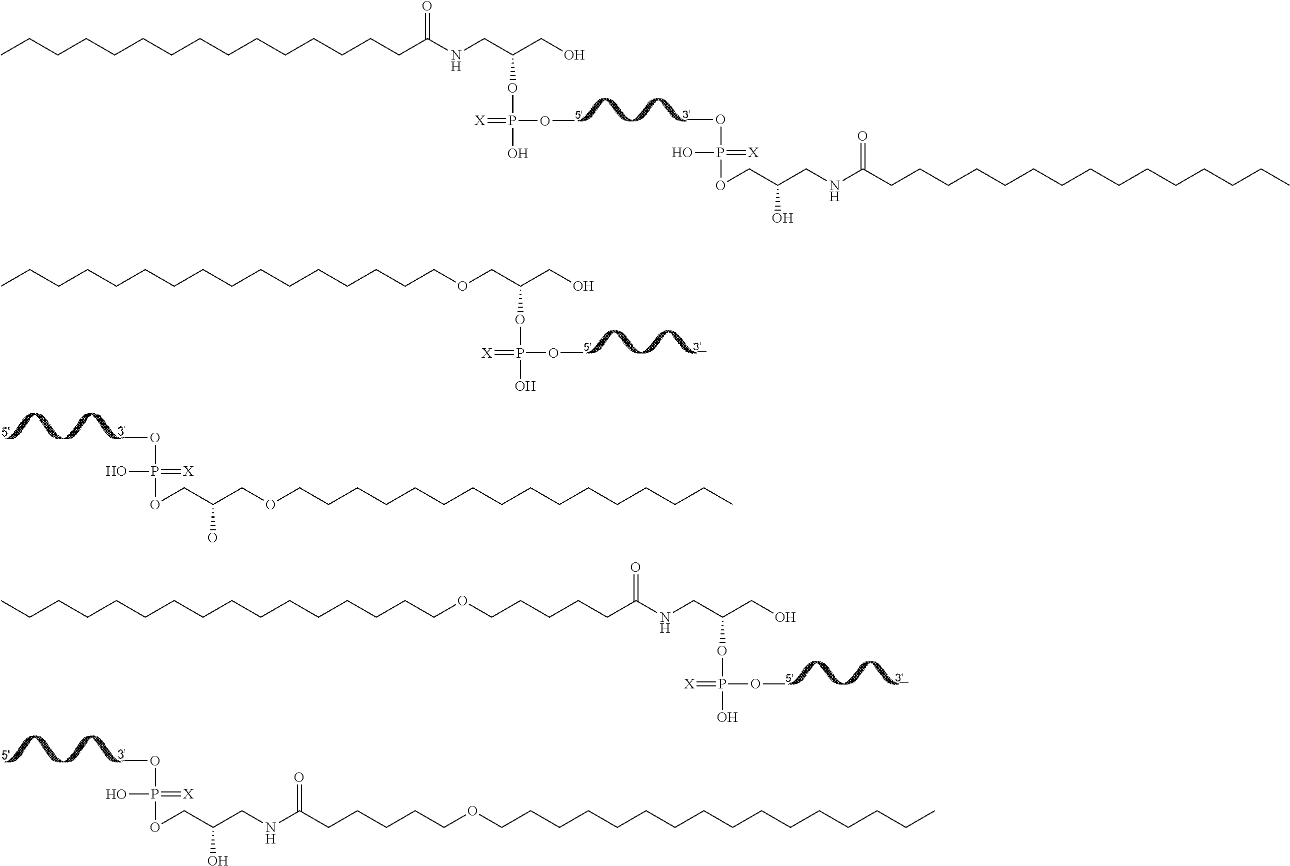

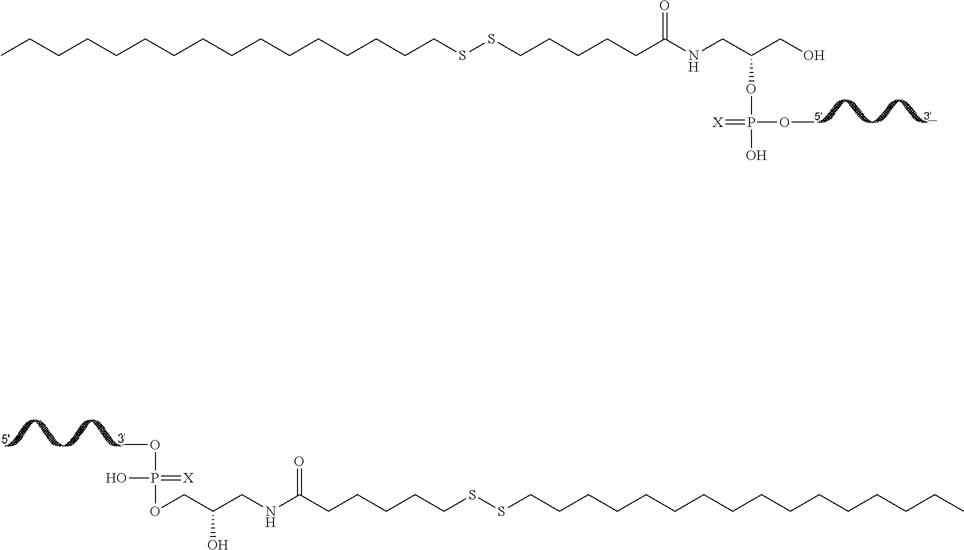

wherein
   X═O or S; and the ribbon represents the sense strand of siRNA or antisense oligonucleotide (ASO);
for example,

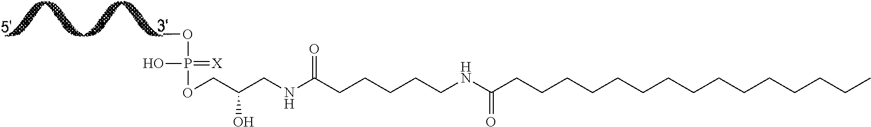

wherein X═O or S; and the ribbon represents the sense strand of siRNA or antisense oligonucleotide (ASO).

In some embodiments,

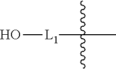

of the sense strand of siRNA or antisense oligonucleotide (ASO) is in accordance with any one of the following formulae:

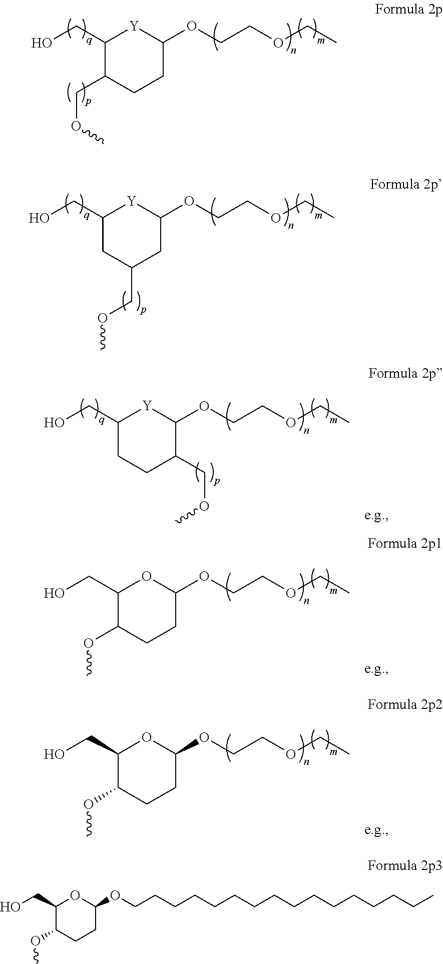

wherein,
Y is O, S, NH or CH₂;
p=0-10, e.g. 0-2;
q=0-10, e.g. 1-2;
n=0-10;
m=1-30, e.g., 11-21, e.g. 13-17; e.g. 15.

In some embodiments,

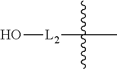

of the sense strand of siRNA or antisense oligonucleotide (ASO) is in accordance with any one of the following formulae:

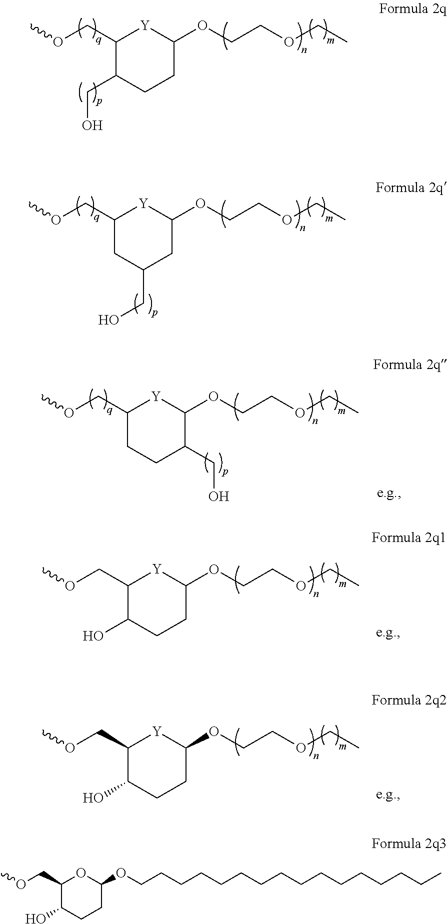

wherein,
Y is O, S, NH or CH₂;
p=0-10, e.g. 0-2;
q=0-10, e.g. 1-2;
n=0-10;
m=1-30, e.g., 11-21, e.g. 13-17; e.g. 15.

In some embodiments,

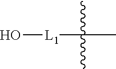

of the sense strand of siRNA or antisense oligonucleotide (ASO) is in accordance with any one of the following formulae:

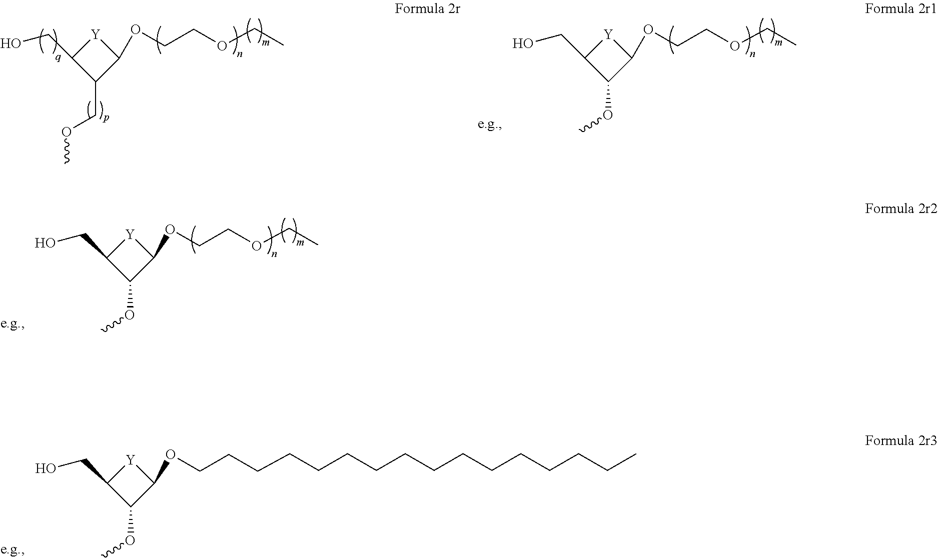

wherein,
Y is O, S, NH or CH₂;
p=0-10, e.g. 0-2;
q=0-10, e.g. 1-2;
n=0-10;
m=1-30, e.g., 11-21, e.g. 13-17; e.g. 15.

In some embodiments,

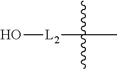

of the sense strand of siRNA or antisense oligonucleotide (ASO) is in accordance with any one of the following formulae:

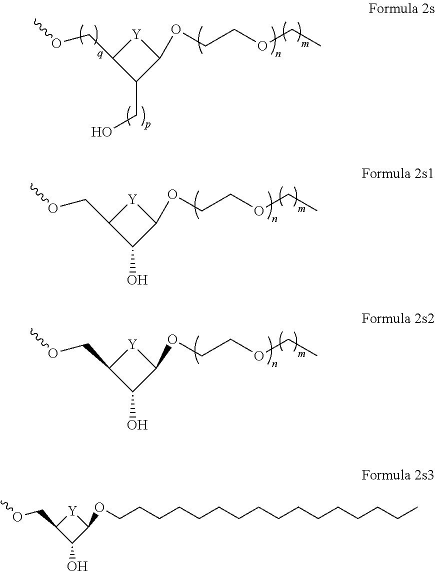

wherein,
Y is O, S, NH or CH₂;
p=0-10, e.g. 0-2;
q=0-10, e.g. 1-2;
n=0-10;
m=1-30, e.g., 11-21, e.g. 13-17; e.g. 15.

In some embodiments,

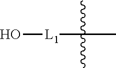

of the sense strand of siRNA or antisense oligonucleotide (ASO) is in accordance with any one of the following formulae:

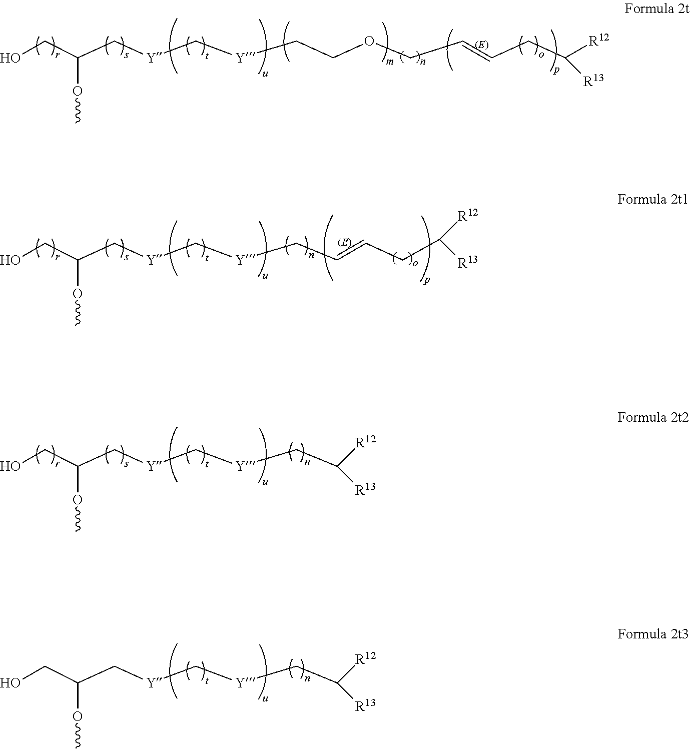

### Wherein,

R₁₂ and R₁₃ are each independently selected from H, halogen, and C₁-C₆ alkyl, optionally substituted with one or more halogen and/or hydroxy;
Y″ is O, S, CH₂, NH, —NHCO—, —CONH—, or —SS—;
r=1-10, e.g. 1-3;
s=1-10, e.g. 1-3;
t=1-10;
u=0-3;
m=0-5;
n=1-30;
o=1-5.

In some embodiments,

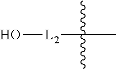

of the sense strand of siRNA or antisense oligonucleotide (ASO) is in accordance with any one of the following formulae:

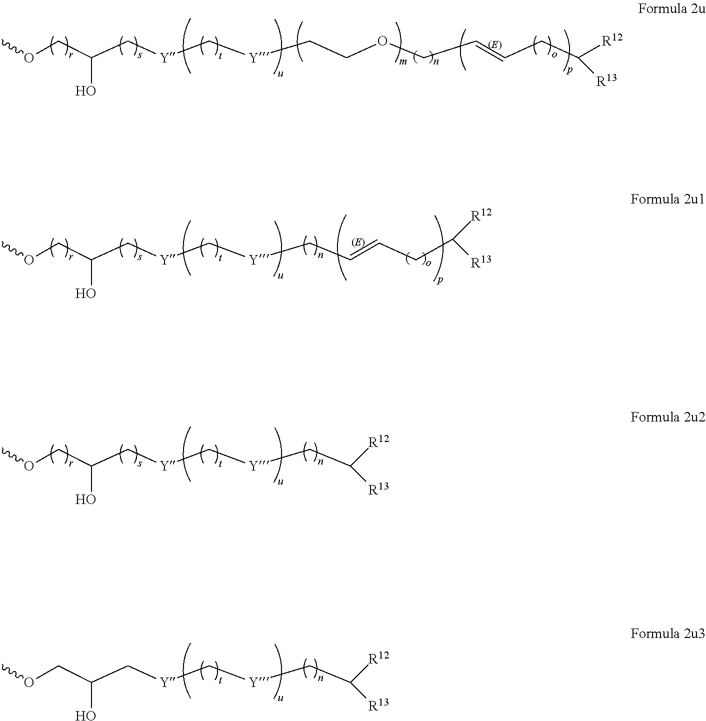

wherein,
R₁₂ and R₁₃ are each independently selected from H, halogen, and C₁-C₆ alkyl, optionally substituted with one or more halogen and/or hydroxy;
Y″ is O, S, CH₂, NH, —NHCO—, —CONH—, or —SS—;
Y′″ is O, S, CH₂, NH, —NHCO—, —CONH, or —SS—;
r=1-10, e.g. 1-3;
s=1-10, e.g. 1-3;
t=1-10;
u=0-3;
m=0-5;
n=1-30;
o=1-5.

In some embodiments, exemplary structures include:

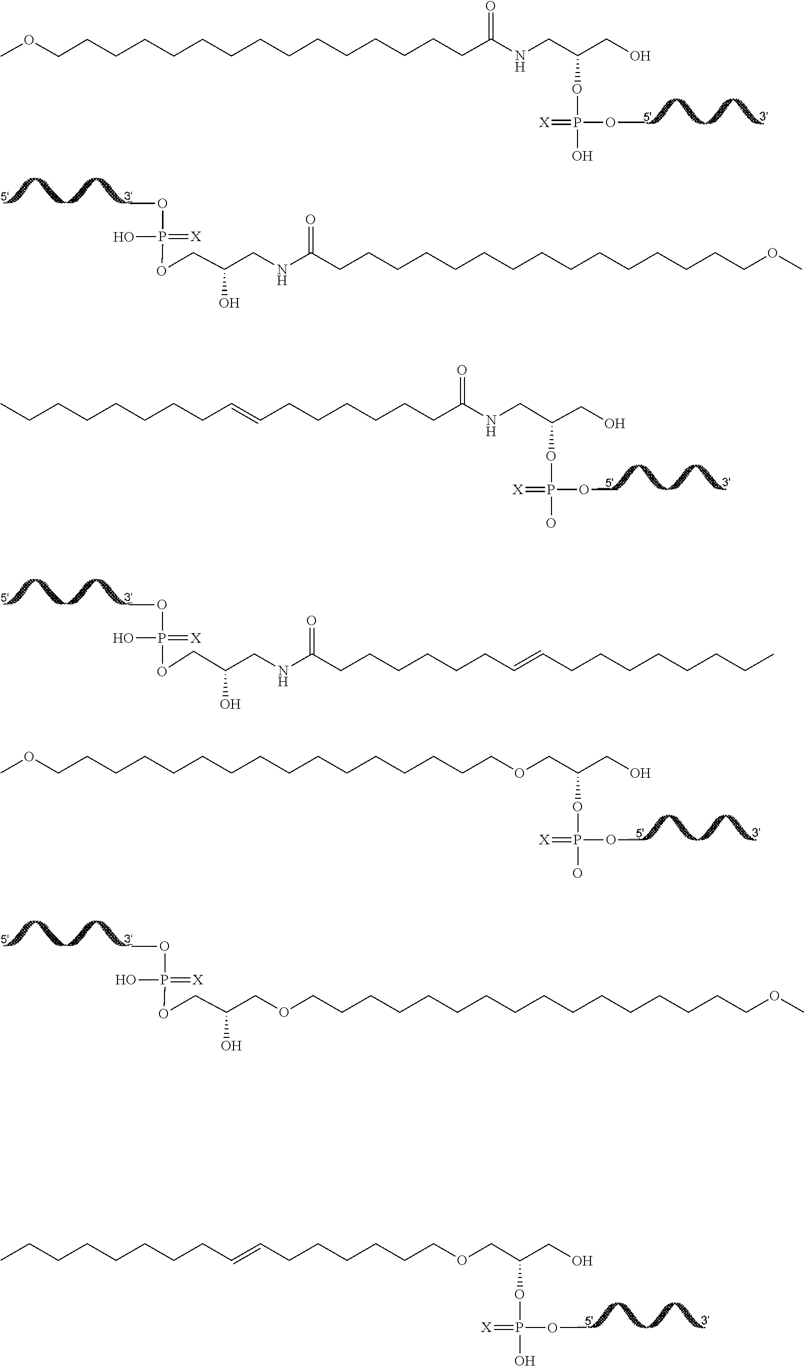

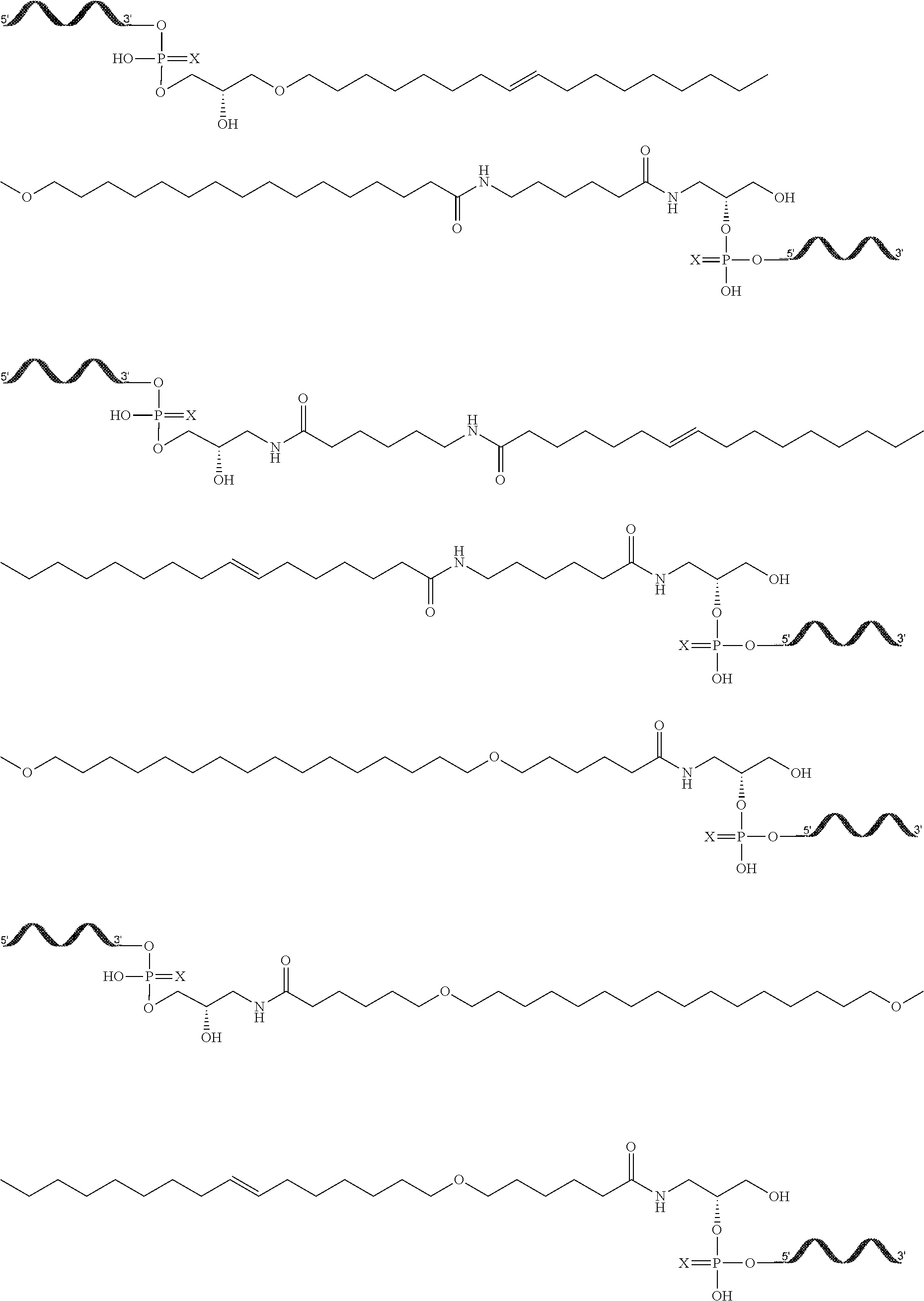

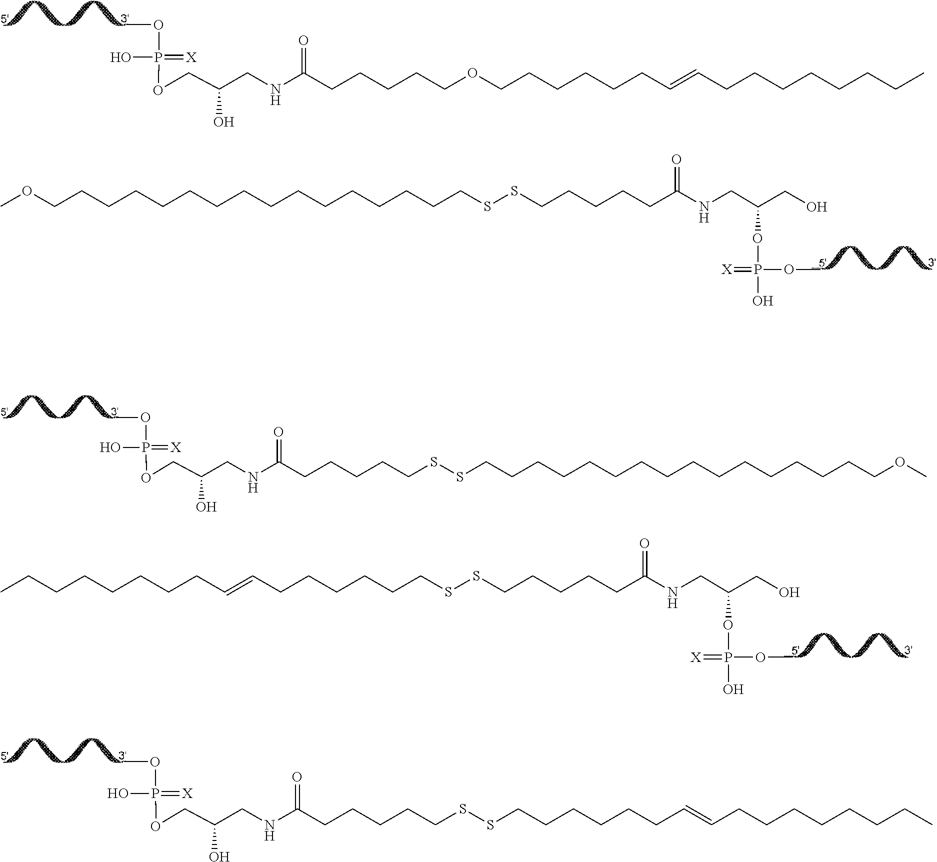

wherein,
X═O or S; and the ribbon represents the sense strand of siRNA or antisense oligonucleotide (ASO).

In some embodiments, the compound (Compound A) contains one or more of the following modifications to the sense and/or antisense strands:
a. Any foregoing compound wherein the sense and antisense nucleotide sequences have a length of 18-23 nucleotides, wherein the sequences are offset by 1, 2 or 3 nucleotides and/or wherein one strand is 1, 2, or 3 nucleotides longer than the other, so that there is an overhang at one or both ends,
b. Any foregoing compound wherein the sense strand is 19 nucleotides in length and the antisense strand is 21 nucleotides in length, or the sense strand is 20 nucleotides in length and the antisense strand is 22 nucleotides in length, or the sense strand is 21 nucleotides in length and the antisense strand is 23 nucleotides in length, such that the siRNA duplex has a two-nucleotide overhang, e.g., wherein the overhang extends beyond the 5′ end of the sense sequence, e.g., as depicted on FIG. 1.
c. Any of the preceding compounds, one or both sequences have a phosphorylated 5′ end.
d. Any of the preceding compounds, wherein the siRNA duplex comprises at least one nucleotide overhang extending beyond the 5′ end of the sense sequence, optionally wherein the overhang comprises two nucleotides.
e. Any of the preceding compounds, wherein the siRNA duplex comprises one or both sequences modified with a phosphonate at the 5′ end.
f. Any of the preceding compounds, wherein the siRNA duplex comprises one or both sequences modified with off-target modifications, e.g., UNA and/or GNA, optionally wherein the modification is present between nucleotides 2-8 of the antisense sequence when read from 5′ to 3′.
g. Any of the preceding compounds, wherein the siRNA duplex comprises one or both sequences modified with one or more lipophilic moieties, optionally wherein the one or more lipophilic moieties is conjugated to one or more internal positions in the double stranded region of the RNAi agent, e.g, wherein the one or more lipophilic moieties are selected from alkyl, e.g. C₁₂₋₂₂ alkyl.
h. Any of the preceding compounds, wherein the siRNA duplex comprises one or both sequences modified with one or more lipophilic moieties and wherein the lipophilic moieties are conjugated to the RNAi agent with or without a linker or carrier; e.g, wherein the one or more lipophilic moieties are selected from alkyl, e.g. C₁₂₋₂₂ alkyl.
i. Any of the preceding compounds, wherein the sense strand of the siRNA duplex is modified at one or both ends with one or more lipophilic moieties conjugated to the siRNA with or without a linker or carrier; e.g, wherein the one or more lipophilic moieties are selected from alkyl, e.g. C₁₂₋₂₂ alkyl.
j. Any of the preceding compounds, wherein the siRNA duplex comprises one or more sequences modified with one or more phosphorothioate linkages between nucleotides.
k. Any of the preceding compounds, wherein the RNAi agent comprises one or more sequences modified with one or more unnatural and/or modified nucleotide, for example 2′-deoxy-2′-fluoro and/or 2′-O-methyl ribosugar modifications.
l. Any of the preceding compounds, wherein the RNAi agent is encapsulated within a delivery carrier, such as a lipid nanoparticle or modified viral capsid.
m. Any of the preceding compounds for use as a pharmaceutical, e.g., for use to reduce expression of the protein product of a target gene in a disease or condition characterized by over-expression, over-activity, or over-accumulation of the protein product of a target gene.

For example, in an embodiment, the disclosure provides any foregoing compound which is a RNAi agent to inhibit LRRK2, VEGFA, or ANGPT2, e.g., a compound selected from any of Compound 1, et seq., wherein the sense strand or antisense strand comprises one or more lipophilic moieties L₁ or L₂ conjugated to the siRNA via a phosphodiester or phosphorothioate bond (e.g. via a ribosyl moiety linked to a phosphodiester or phosphorothioate bond) to the 3′ terminal of the sense strand, e.g., as described in the foregoing scopes.

In one embodiment, the disclosure thus provides a compound (Compound 1), which is a double-stranded siRNA duplex, comprising a sense sequence and a complementary antisense sequence, or an antisense oligonucleotide (ASO) wherein
a) the compound comprises one or more sequences corresponding to a region of a LRRK2 gene, e.g., wherein the compound is effective to significantly reduce LRRK2 mRNA in a cell, e.g., as determined cell-based assay (e.g., in an assay substantially as described in Example 3), e.g., at a concentration of 10 nM or less;; or
b) the compound comprises one or more sequences corresponding to a region of a VEGFA gene, e.g., wherein the compound is effective to significantly reduce VEGFA mRNA in a cell, e.g., as determined cell-based assay, e.g., at a concentration of 10 nM or less; or
c) the compound comprises one or more sequences corresponding to a region of ANGPT2 gene, wherein the compound is effective to significantly reduce ANGPT2 mRNA in a cell, e.g., as determined cell-based assay, e.g., at a concentration of 10 nM or less.

For example, in particular embodiments the disclosure provides:
1.1. Compound 1, wherein the compound provides at least 70% mRNA knockdown activity at 10 nM in a cell-based assay.
1.2. Any foregoing compound, wherein the compound provides at least 70% mRNA knockdown activity at 1 nM in a cell-based assay.
1.3. Any foregoing compound, wherein the compound provides at least 70% mRNA knockdown activity at 0.1 nM in a cell-based assay.
1.4. Any foregoing compound, wherein the compound provides at least 40% mRNA knockdown activity at 0.01 nM.
1.5. Any foregoing compound, wherein the RNAi agent comprises a sequence with complementarity to a LRRK2 gene.
1.6. Any foregoing compound, wherein the RNAi agentcomprises a sequence with complementarity to a LRRK2 gene with one or more mutations associated with a disease, disorder, or pathology.
1.7. Any foregoing compound, wherein the sense or antisense strand comprises a sequence selected from any one of SEQ ID NOS. 1-403, e.g., SEQ ID NOS. 1-200; e.g., a duplex as set forth on Table 1; e.g. a duplex as set forth on Table 1 other than N3T130025-01.
1.8. Any foregoing compound, wherein the sense or antisense strand comprises a sequence selected from any one of the sequences listed in Tables 1, 2a, 2b, 4, 5, 7, 9, 13, 16, 18, 20, 22, 24, 26, 29, 33.
1.9. Any foregoing compound comprising a sense or antisense sequence selected from the sequences listed in Table 5.
1.10. Any foregoing compound selected from the duplexes listed in Table 5.
1.11. Any foregoing compound, wherein the antisense strand comprises a sequence that is complementary to a sequence containing, or within three or fewer nucleotide positions from, one of the following nucleotide positions of mRNA LRRK2 (numbering based on gene sequence disclosed in NCBI refseq ID NG_011709.2; NCBI GeneID: 120892): 169, 377, 864, 868, 1807, 2279, 2429, 3255, 3347, 3731, 3739, 3803, 3804, 3818, 4324, 4597, 5226, 5386, 5387, or 6839; e.g. 377, 868, 1807, 3255, 3739, 3818, 4324, 5226, 5386, 5387, or 4324; e.g. 868 or 3255.
1.12. Any foregoing compound, wherein the antisense strand comprises a sequence that is complementary or capable of hybridizing to sequence on the LRRK2 mRNA within a hotspot as identified in Table 2a.
1.13. Any foregoing compound wherein the sense and antisense nucleotide sequences have a length of 18-23 nucleotides, wherein the sequences are offset by 1, 2 or 3 nucleotides and/or wherein one strand is 1, 2, or 3 nucleotides longer than the other, so that there is an overhang at one or both ends,
1.14. Any foregoing compound wherein the sense strand is 19 nucleotides in length and the antisense strand is 21 nucleotides in length, or the sense strand is 20 nucleotides in length and the antisense strand is 22 nucleotides in length, or the sense strand is 21 nucleotides in length and the antisense strand is 23 nucleotides in length, such that the siRNA duplex has a two-nucleotide overhang, e.g., wherein the overhang extends beyond the 5′ end of the sense sequence, e.g., as depicted on FIG. 1.
1.15. Any of the preceding compounds, one or both sequences have a phosphorylated 5′ end.
1.16. Any of the preceding compounds, wherein the siRNA duplex comprises at least one nucleotide overhang extending beyond the 5′ end of the sense sequence, optionally wherein the overhang comprises two nucleotides.
1.17. Any of the preceding compounds, wherein the RNAi agent comprises one or both sequences modified with a phosphonate at the 5′ end.
1.18. Any of the preceding compounds, wherein the RNAi agent comprises one or both sequences modified with off-target modifications, e.g., UNA and/or GNA, optionally wherein the modification is present between nucleotides 2-8 of the antisense sequence when read from 5′ to 3′.
1.19. Any of the preceding compounds, wherein the RNAi agent comprises one or both sequences modified with one or more lipophilic moiety, optionally wherein the one or more lipophilic moiety is conjugated to one or more internal positions in the double stranded region of the siRNA duplex.
1.20. Any of the preceding compounds, wherein the RNAi agent comprises one or both sequences modified with one or more lipophilic moiety and wherein the lipophilic moiety is conjugated to the siRNA with or without a linker or carrier.
1.21. Any of the preceding compounds, wherein the sense strand of the RNAi agent is modified at one or both ends with one or more lipophilic moieties conjugated to the siRNA with or without a linker or carrier.
1.22. Any of the preceding compounds, wherein the sense strand of the siRNA duplex is modified at one or both ends with one or more lipophilic moieties conjugated to the siRNA with or without a linker or carrier in accordance with any of Compound A, et seq.
1.23. Any of the preceding compounds, wherein the RNAi agent comprises one or more sequences modified with one or more phosphorothioate linkages between nucleotides.
1.24. Any of the preceding compounds, wherein the RNAi agent comprises one or more sequences modified with one or more unnatural and/or modified nucleotide, for example 2′-deoxy-2′-fluoro and/or 2′-O-methyl ribosugar modifications.
1.25. Any of the preceding compounds, wherein the RNAi agent comprises one or more sequences modified with one or more of the modifications described in any of paragraphs [0053]-[0085].
1.26. Any of the preceding compounds, wherein the RNAi agent is encapsulated within a delivery carrier, such as a lipid nanoparticle or modified viral capsid.
1.27. Any of the preceding compounds for use as a pharmaceutical.
1.28. Any of the preceding compounds wherein the compound comprises one or more sequences corresponding to a region of a LRRK2 gene, for use in treating a LRRK2-associated disease, disorder, or pathology, for example inflammatory bowel disease (IBD), Crohn's disease (CD), ulcerative colitis (UC), susceptibility to bacterial infections such as Hansen's disease/leprosy, and/or Parkinson's disease (PD).
1.29. Any of the preceding compounds wherein the compound comprises one or more sequences corresponding to a region of a LRRK2 gene, for use in treating a disease, disorder, or pathology associated with elevated Lrrk2 activity, e.g., Crohn's disease (CD), inflammatory bowel disease (IBD), ulcerative colitis (UC), and/or Parkinson's disease (PD).
1.30. Any of the preceding compounds wherein the compound comprises one or more sequences corresponding to a region of a LRRK2 gene, for use in treating a condition associated with a mutation of LRRK2.
1.31. Any of the preceding compounds wherein the compound comprises one or more sequences corresponding to a region of a LRRK2 gene, for use in treating a condition associated with Lrrk2 activity, e.g. enhanced Lrrk2 kinase and/or GTPase activity.
1.32. Any of the preceding compounds wherein the compound comprises one or more sequences corresponding to a region of a LRRK2 gene, for use in treating Parkinson's disease or parkinsonism.
1.33. Any of the preceding compounds wherein the compound comprises one or more sequences corresponding to a region of a LRRK2 gene, for use in treating a condition associated with a LRRK2 mutation, e.g. selected from mutations associated with Parkinson's Disease, e.g., at one or more of positions 1371, 1437, 1441, 1699, 2019, and/or 2020, e.g. selected from I1371V, N1437H, N1437D, R1441C, R1441G, R1441H, R1441S, Y1699C, G2019S, and I2020T; e.g., G2019S, I2020T, R1441C, R1441G, and R1441H.
1.34. Any of the preceding compounds wherein the compound comprises one or more sequences corresponding to a region of a LRRK2 gene, for use in any of Methods 2, et seq.
1.35. Any of the preceding compounds wherein the compound comprises one or more sequences corresponding to a region of a VEGFA gene or an ANGPT2 gene, wherein the antisense sequence hybridizes to a sequence selected from SEQ IDS: 201-220, 261-268, 272-285, 356-393, and 394-403; e.g, wherein the antisense sequence comprises a sequence selected in Tables 9-12 and 18-34.
1.36. Any of the preceding compounds wherein the compound comprises one or more sequences corresponding to a region of a VEGFA gene or an ANGPT2 gene, for use in any of Methods 2A, et seq.
1.37. Any of the preceding compounds, comprising one or more seed region modifications (nucleotides 2-8) on the antisense sequence comprising one or more of the following nucleosides:

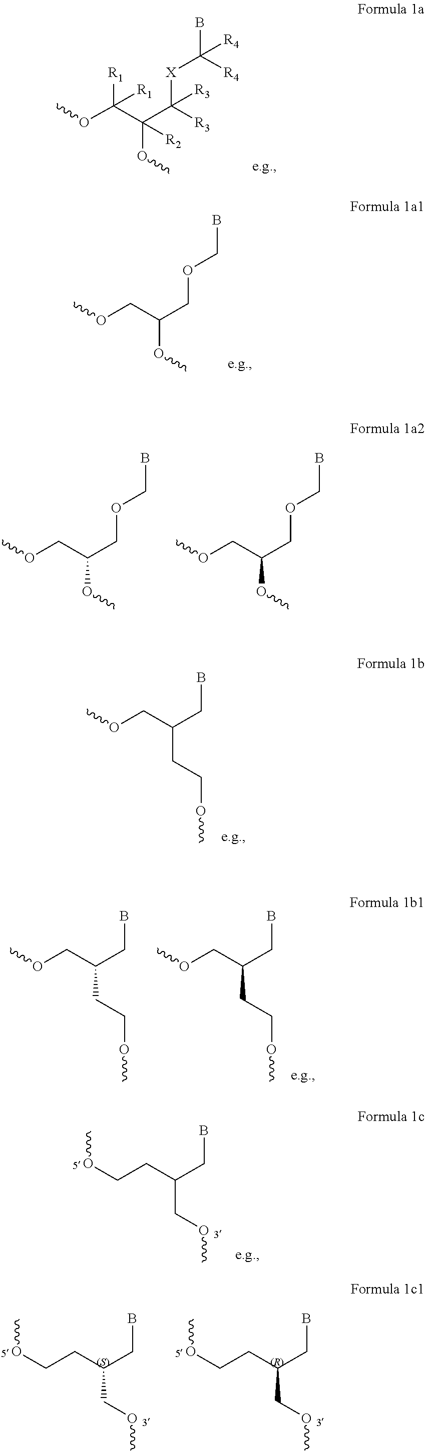

wherein
X═O, S, NH, —CONH—, or —NHCO—; and
B is a modified or unmodified nucleobase; R₁, R₂, R₃ and R₄ are independently H, halogen, OR₅, or alkyl; and R₅ is H, alkyl, cycloalkl, aryl, aralkyl, heteroaryl or sugar;
for an example a double-stranded siRNA duplex, comprising a sense sequence and a complementary antisense sequence, wherein the antisense sequence comprises one or more nucleoside analogues of Formula 1a or Formula 1b, e.g., wherein the antisense sequence comprises one or more nucleoside analogues selected from N3T12-027-01 and N3T12-031-01.
1.41 Any of the preceding compounds, comprising one or more seed region modifications (nucleotides 2-8) on the antisense sequence comprising one or more of the nucleosides of Table B.

TABLE B

| Monomer | Corresponding code in antisense strand |
|---|---|
| [N3T12-027-01-R] | [N3T1-U-R] |
| [N3T12-027-01-S] | [N3T1-U-S] |
| [N3T12-031-01-R] | [N3T1-A-R] |
| [N3T12-031-01-S] | [N3T1-A-S] |
| [N3T12-047-01] | [N3T1-C-R] |
| [N3T12-070-01] | [N3T1-C-S] |
| [N3T12-048-01] | [N3T1-G-R] |
| [N3T12-071-01] | [N3T1-G-S] |

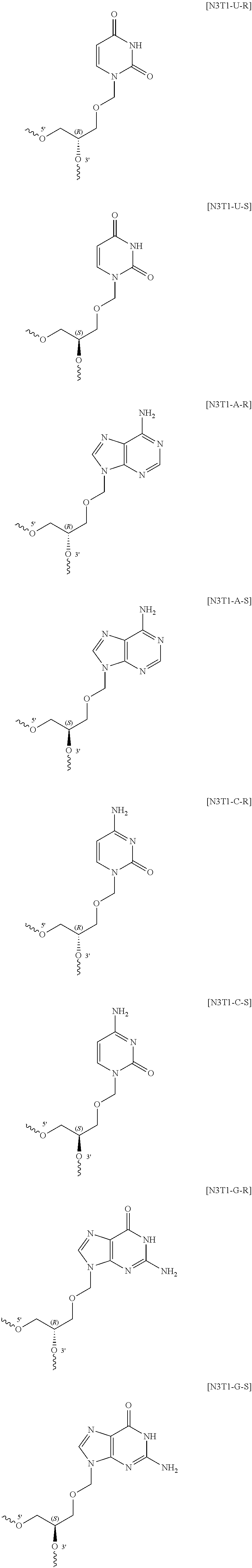

1.42 Any of the preceding compounds, which is a double-stranded siRNA duplex, comprising a sense sequence and a complementary antisense sequence, wherein the antisense sequence comprises a nucleoside analogue of Formula A at the 5′-terminus of Formula A:

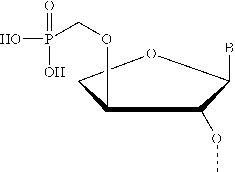

wherein B is a modified or unmodified nucleobase, and the dotted line represents attachment to a terminal phosphate of an oligonucleotide sequence; e.g.,

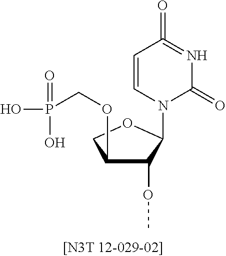

1.43 Any of the preceding compounds, wherein the modified siRNA which is an RNA duplex comprising a sense strand and an antisense strand, wherein the sense strand comprises one or more lipophilic moieties L₁ or L₂ conjugated to the siRNA via a phosphodiester or phosphorothioate bond accordance with Formula 2:

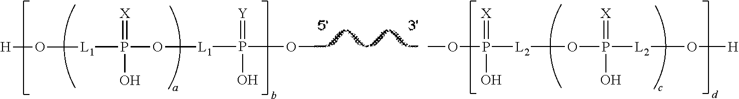

wherein
X═O or S;
a=0-4;
b=0-4;
c=0-4;
d=0-4;
   provided that b and d are not both 0; and the ribbon represents the sense strand.
1.44 Any of the preceding compounds, wherein

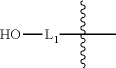

of the sense strand is in accordance with one of the following formulae:

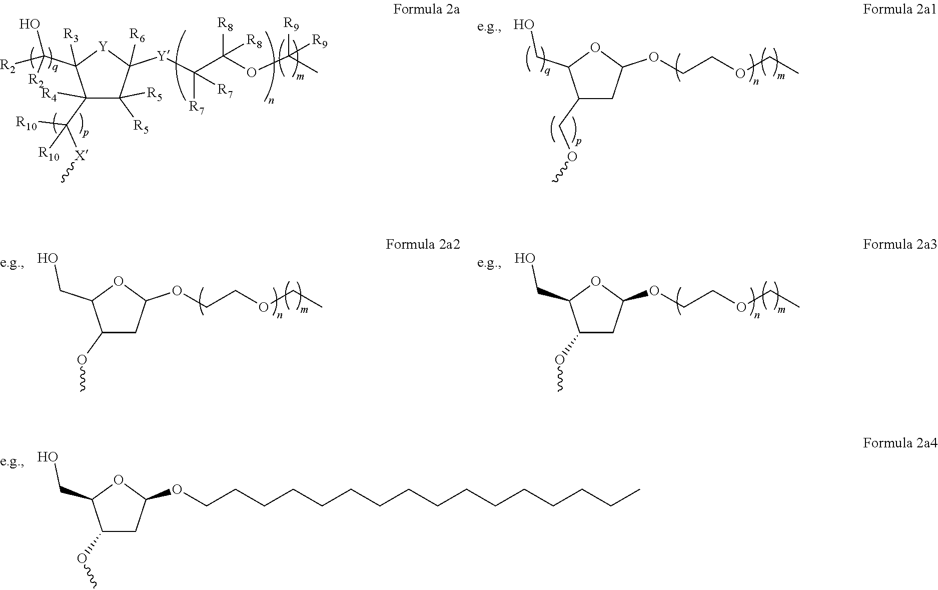

wherein,
R₂, R₃, R₄, R₅, R₆, R₇, R₈, R₉, R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, e.g. 0-2;
q=0-10, e.g. 1-2;
n=0-10;
m=1-30, e.g., 11-21, e.g. 13-17; e.g. 15.;

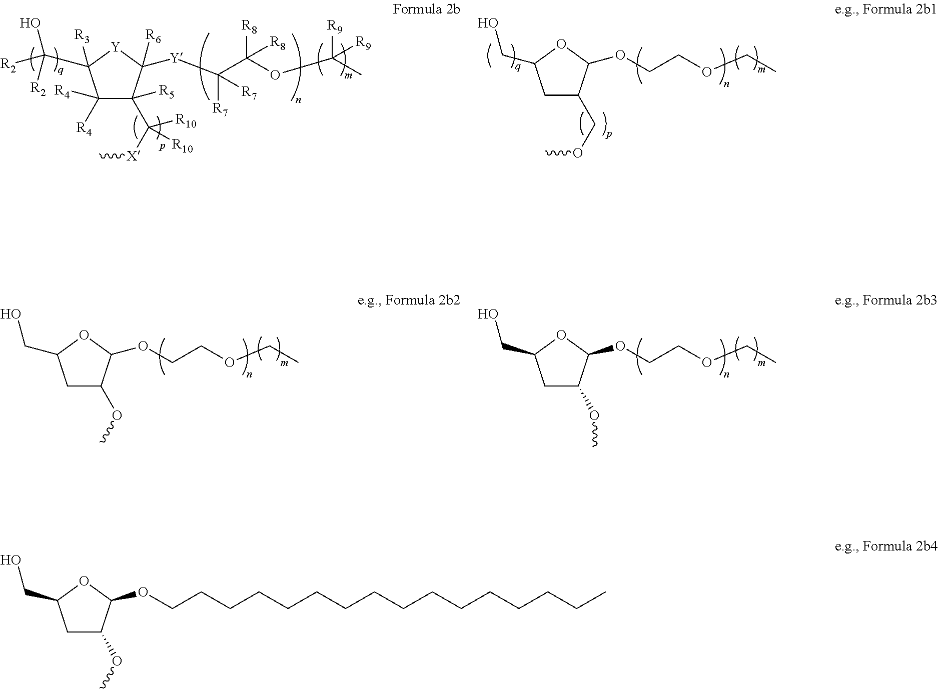

wherein,
R₂, R₃, R₄, R₅, R₆, R₇, R₈, R₉, R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, e.g. 0-2;
q=0-10, e.g. 1-2;
n=0-10;
m=1-30, e.g., 11-21, e.g. 13-17; e.g. 15.
1.41 Any of the preceding compounds, wherein

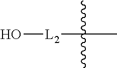

of the sense strand is in accordance with one of the following formulae:

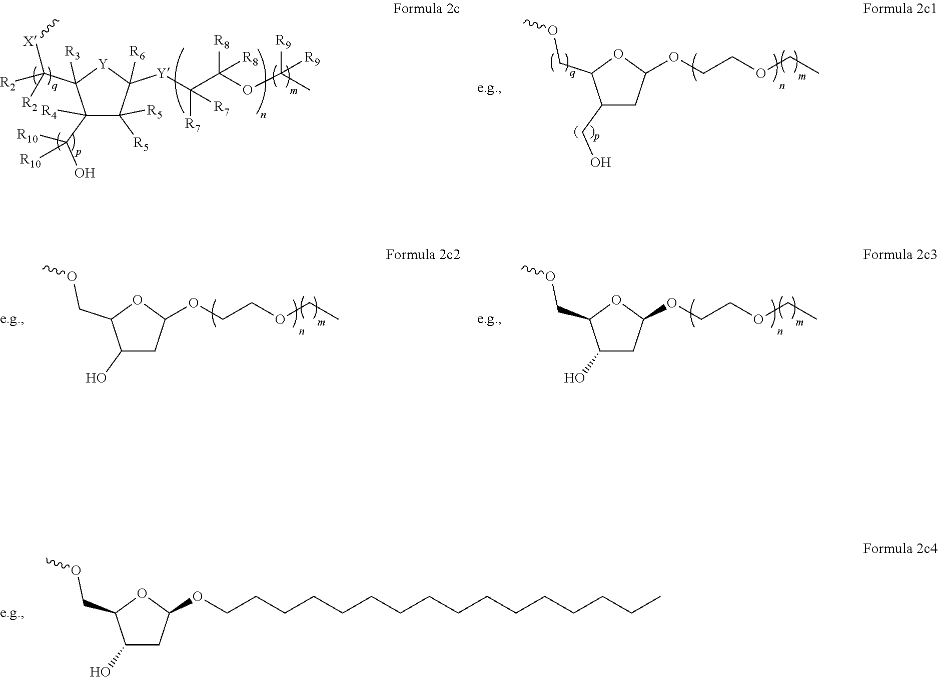

wherein,
R₂, R₃, R₄, R₅, R₆, R₇, R₈, R₉, R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, e.g. 0-2;
q=0-10, e.g. 1-2;
n=0-10;
m=1-30, e.g., 11-21, e.g. 13-17; e.g. 15.;

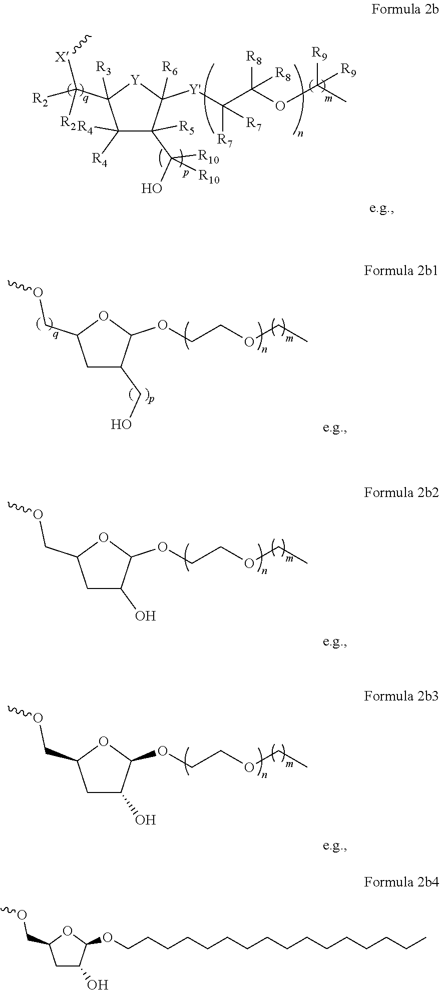

wherein,
R₂, R₃, R₄, R₅, R₆, R₇, R₈, R₉, R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, e.g. 0-2;
q=0-10, e.g. 1-2;
n=0-10;
m=1-30, e.g., 11-21, e.g. 13-17; e.g. 15.
1.42 Any of the preceding compounds, wherein

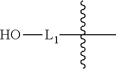

of the sense strand is in accordance with one of the following formulae:

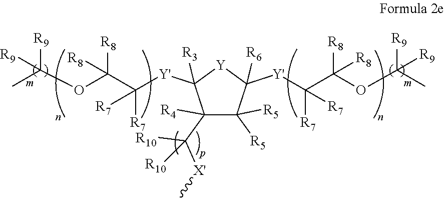

wherein a=0, b=1, c=0, and d=0

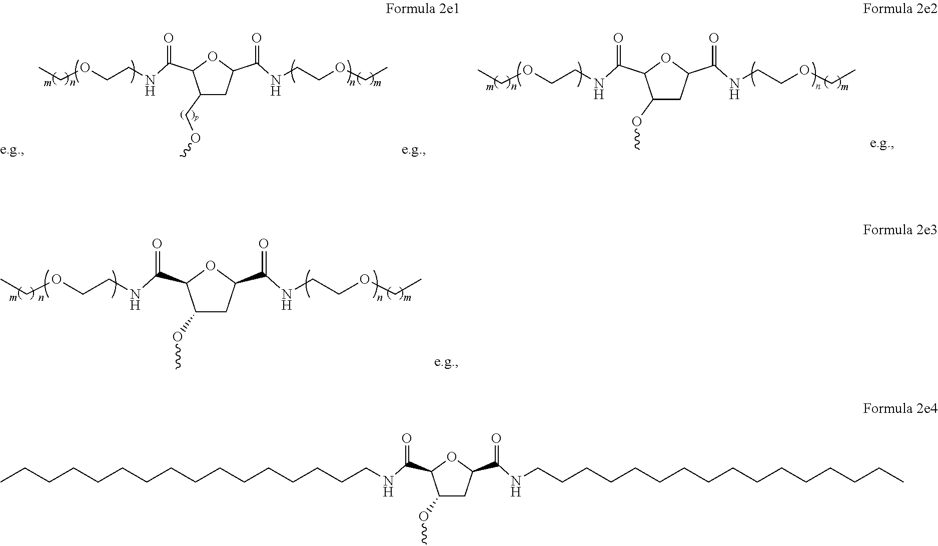

R₃, R₄, R₅, R₆, R₇, R₈, R₉, R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, e.g. 0-2;
n=0-10;
m=1-30, e.g., 11-21, e.g. 13-17; e.g. 15.

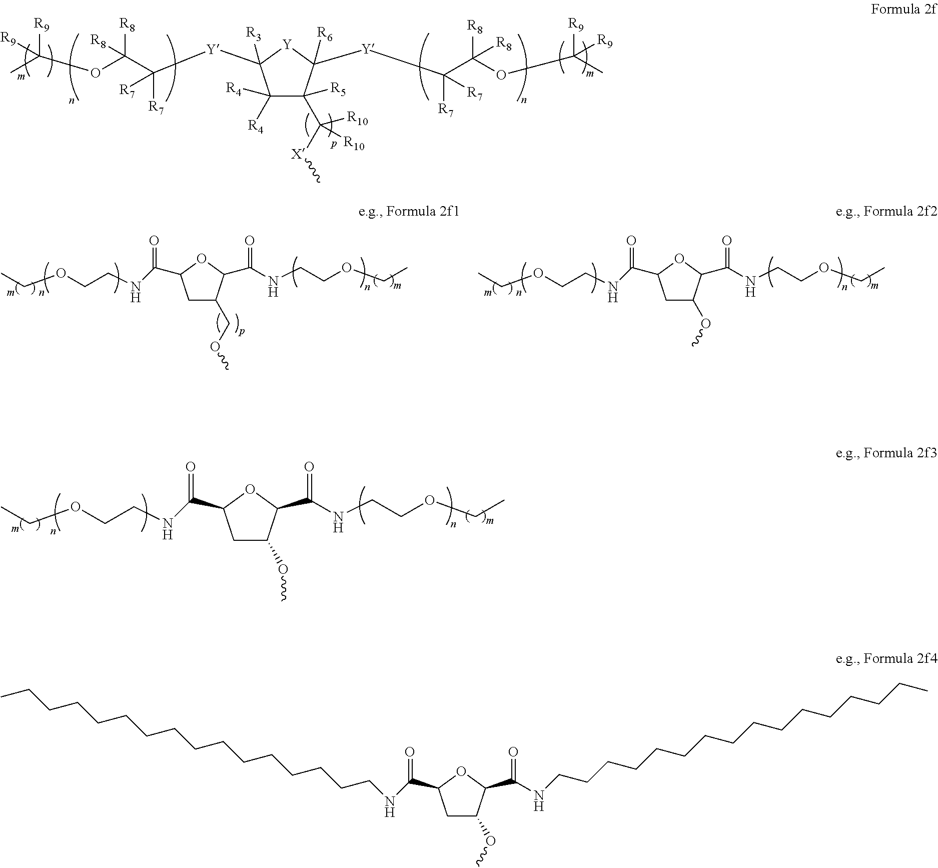

wherein,
R₃, R₄, R₅, R₆, R₇, R₈, R₉, R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, e.g. 0-2;
q=0-10;
n=0-10;
m=1-30, e.g., 11-21, e.g. 13-17; e.g. 15.
1.43 Any of the preceding compounds, wherein

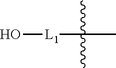

of the sense strand is in accordance with one of the following formulae:

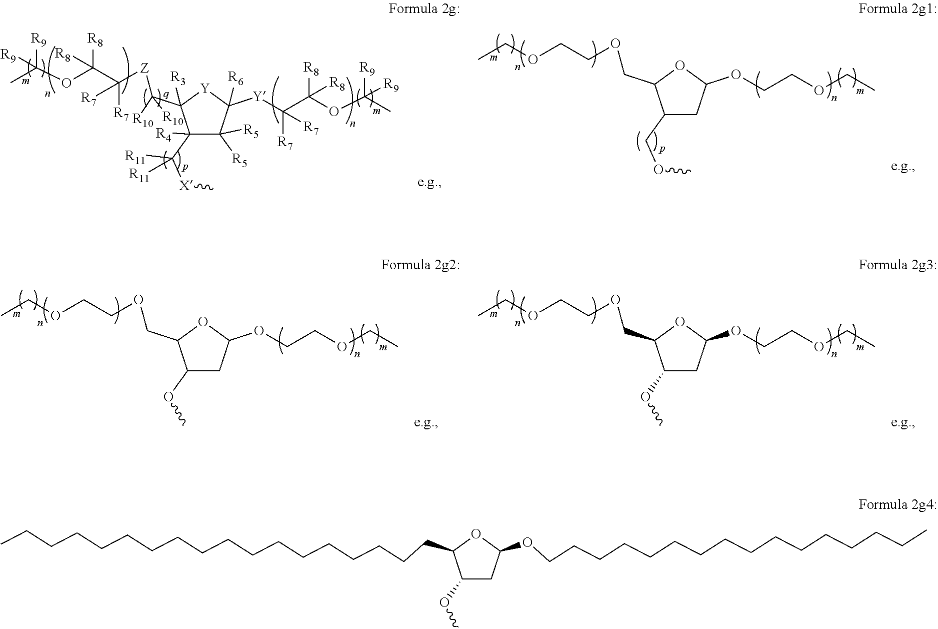

wherein,
R₂, R₃, R₄, R₅, R₆, R₇, R₈, R₉, R₁₀ and R₁₁ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, e.g. 0-2;
q=0-10, e.g. 1-2;
n=0-10;
m=1-30, e.g., 11-21, e.g. 13-17; e.g. 15;

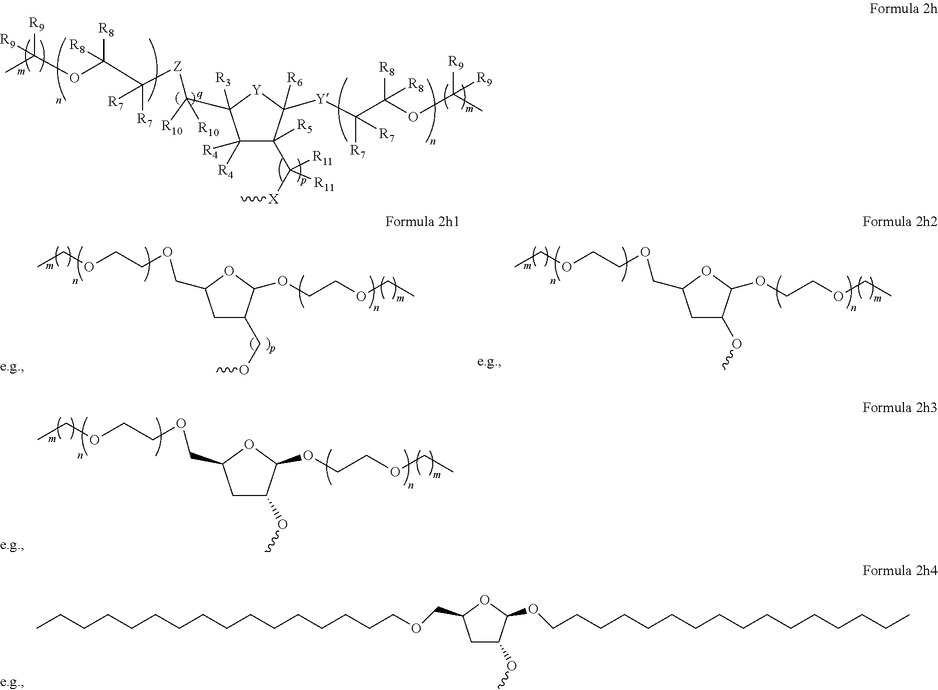

wherein,
R₃, R₄, R₅, R₆, R₇, R₈, R₉, R₁₀ and R₁₁ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, —CH₂O—, —OCH₂—, or —SS—;
Z is O, S, CH₂, NH, —NHCO—, —CONH—, or —SS—;
p=0-10, e.g. 0-2;
q=0-10, e.g. 1-2;
n=0-10;
m=1-30, e.g., 11-21, e.g. 13-17; e.g. 15;

provided that the O with the wavy line on said L₁ or L₂ is part of a phosphodiester or phosphorothiate bond to the sense strand;

for example,

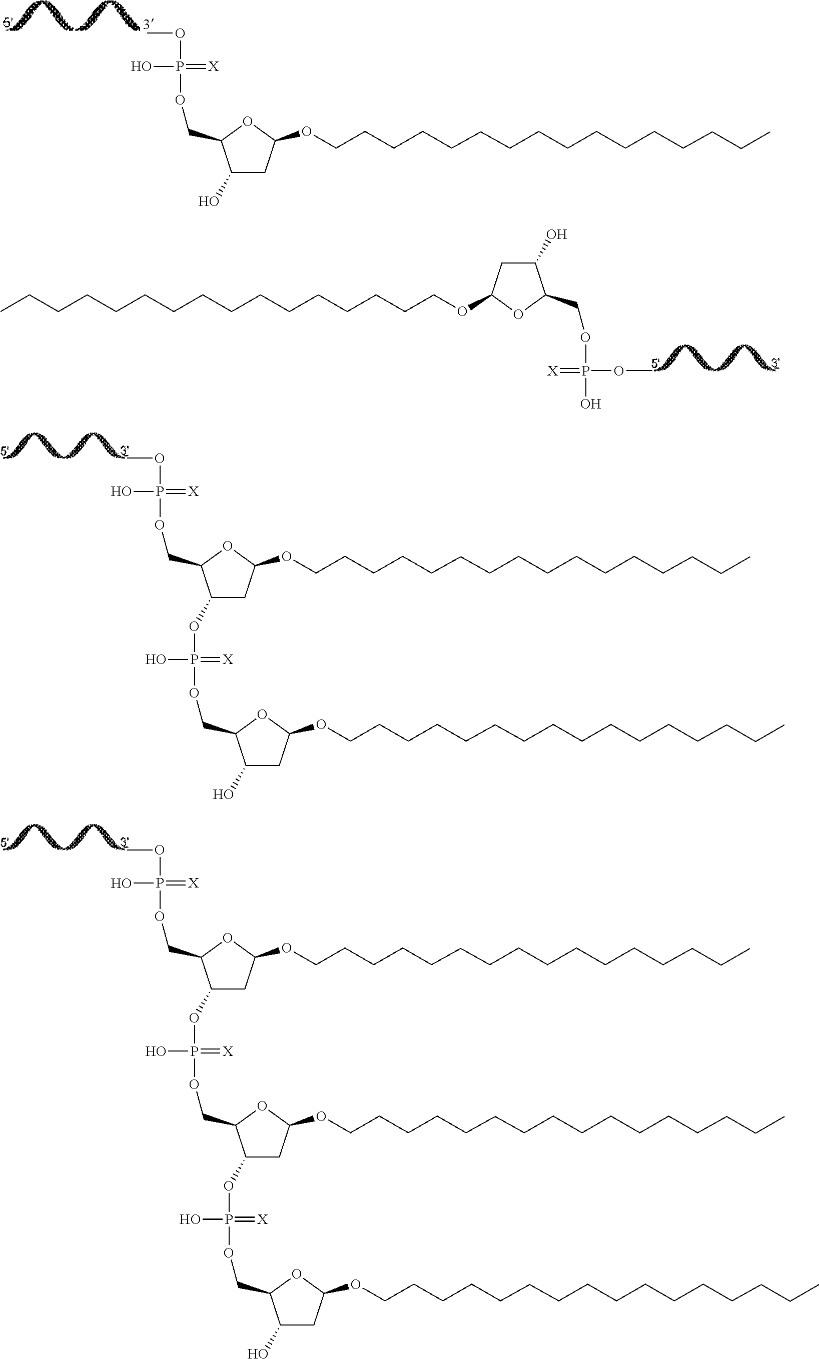

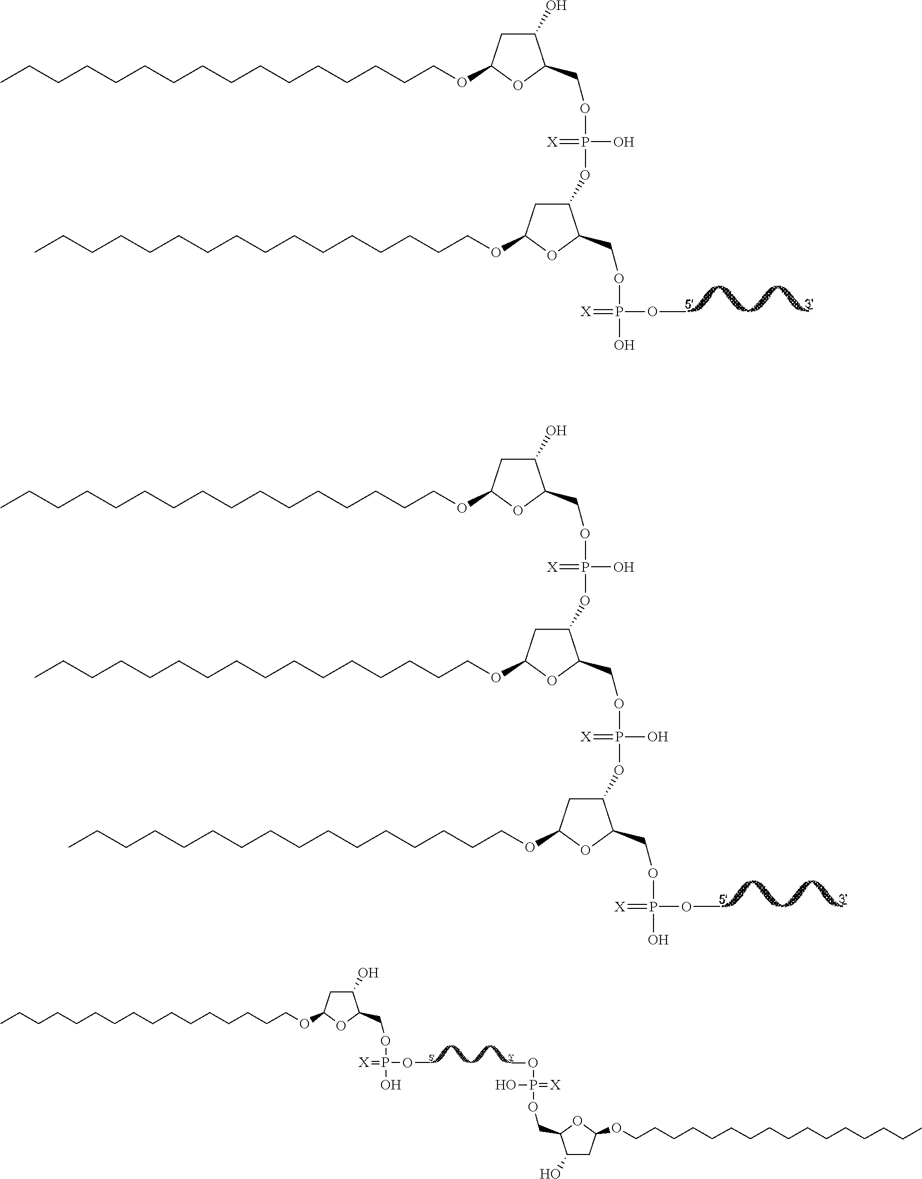

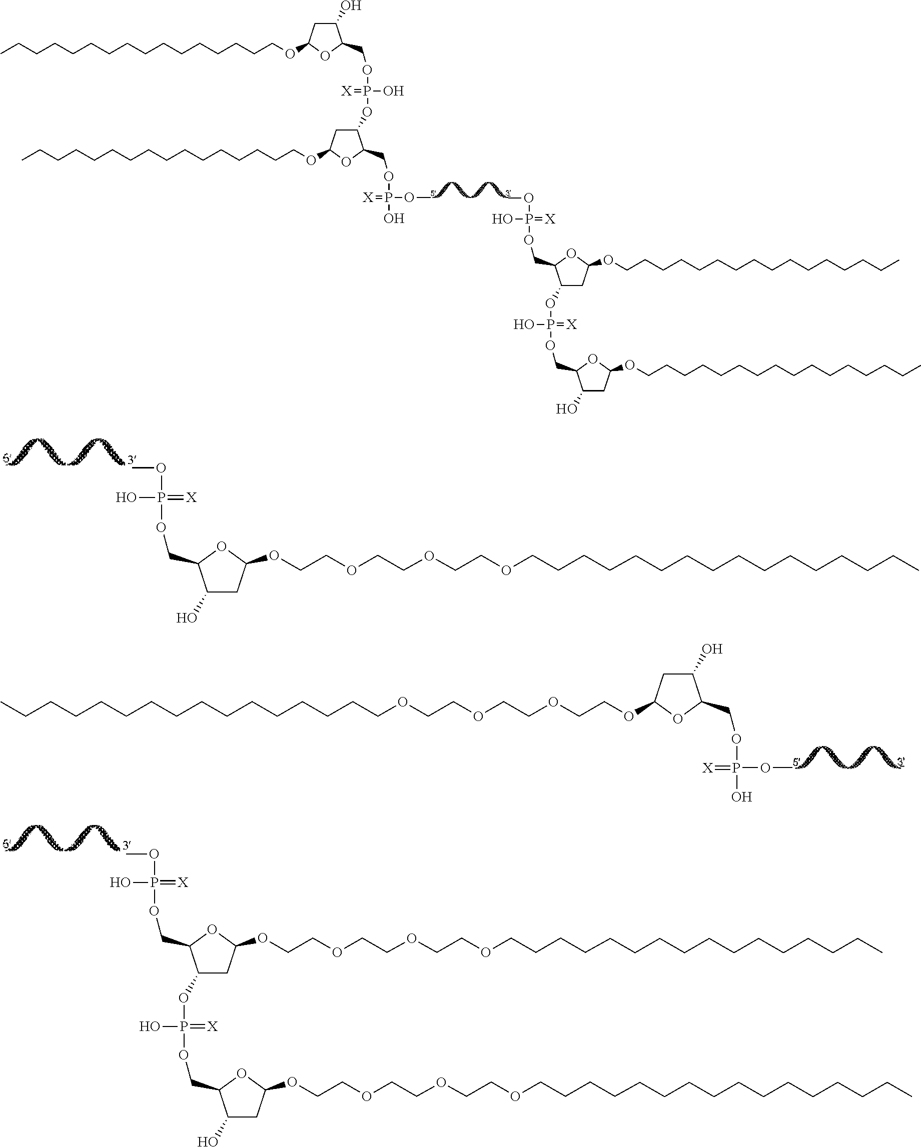

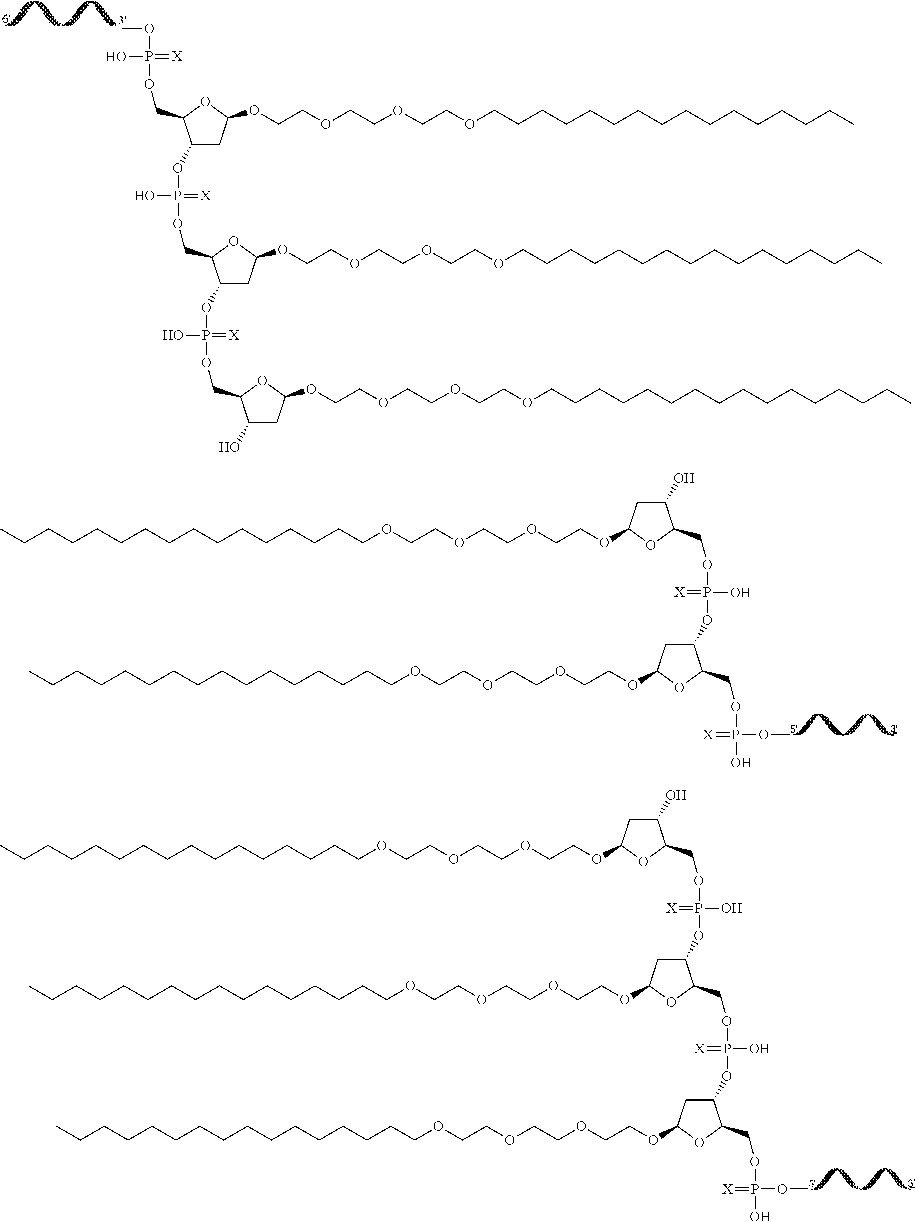

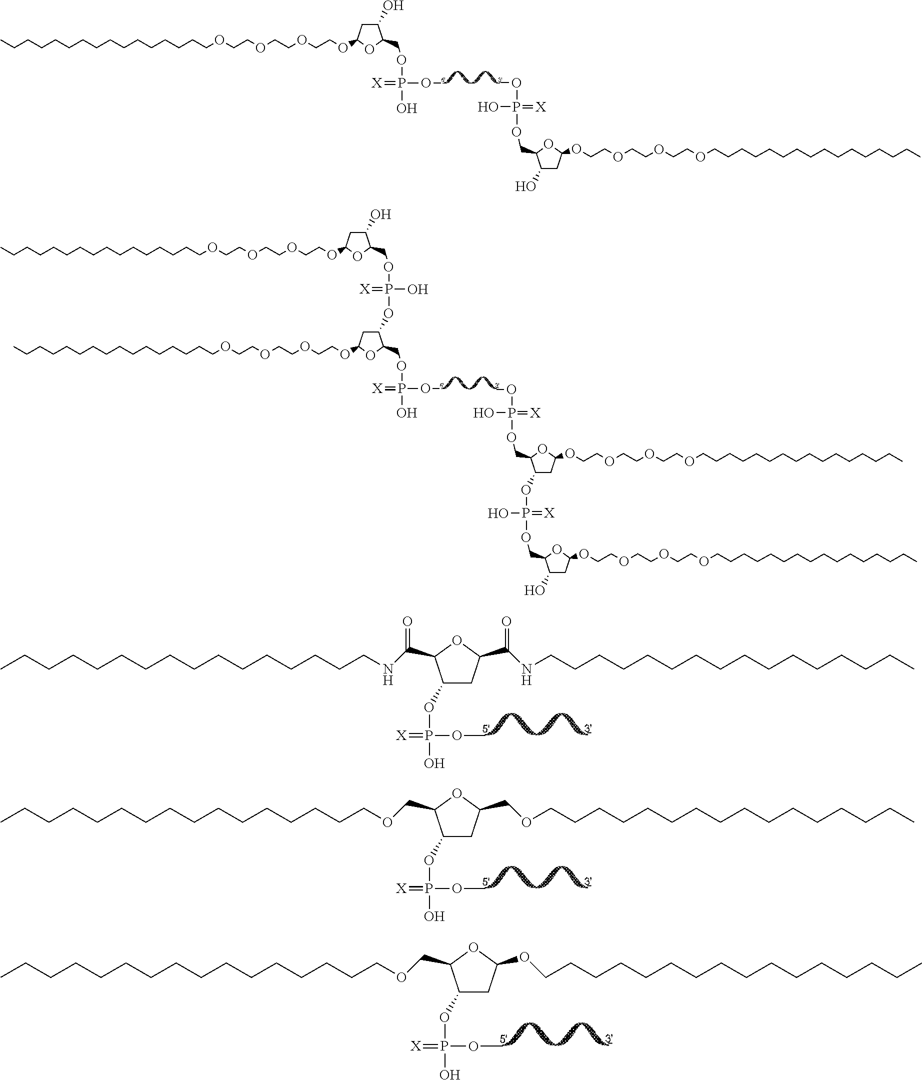

wherein
X═O or S; and the ribbon represents the sense strand.
1.44 Any of the preceding compounds, wherein selected nucleotides are linked to 2′-O lipid conjugate moieties or adamantyl groups on sense strand, in accordance with one of the following formulae:

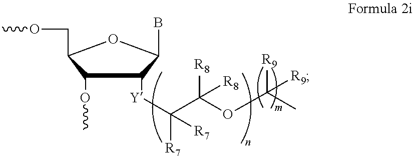

for example:

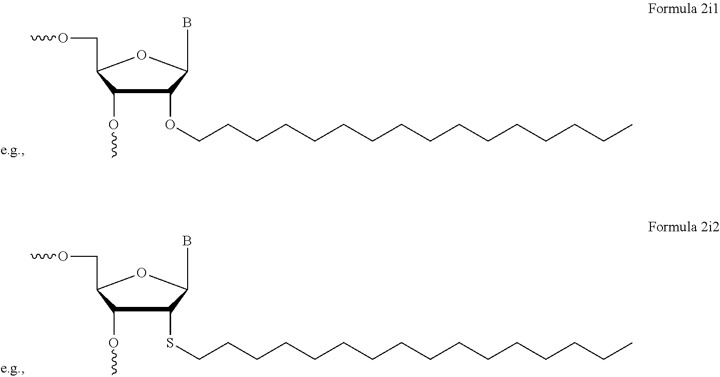

wherein,
R₇, R₈ and R₉ are each independently H, halogen, or C1-C6 alkyl, optionally substituted with one or more halogen and/or hydroxy;
Y′ is O, S, CH₂, NH, —NHCO—, —CONH—, —CH₂O—, or —SS—;
n=0-10;
m=1-30, e.g., 11-21, e.g. 13-17; e.g. 15.
1.45 Any of the preceding compounds wherein selected nucleotides are linked to 5′O-lipid conjugate moieties or adamantyl groups on sense strand, in accordance with one of the following formulae:

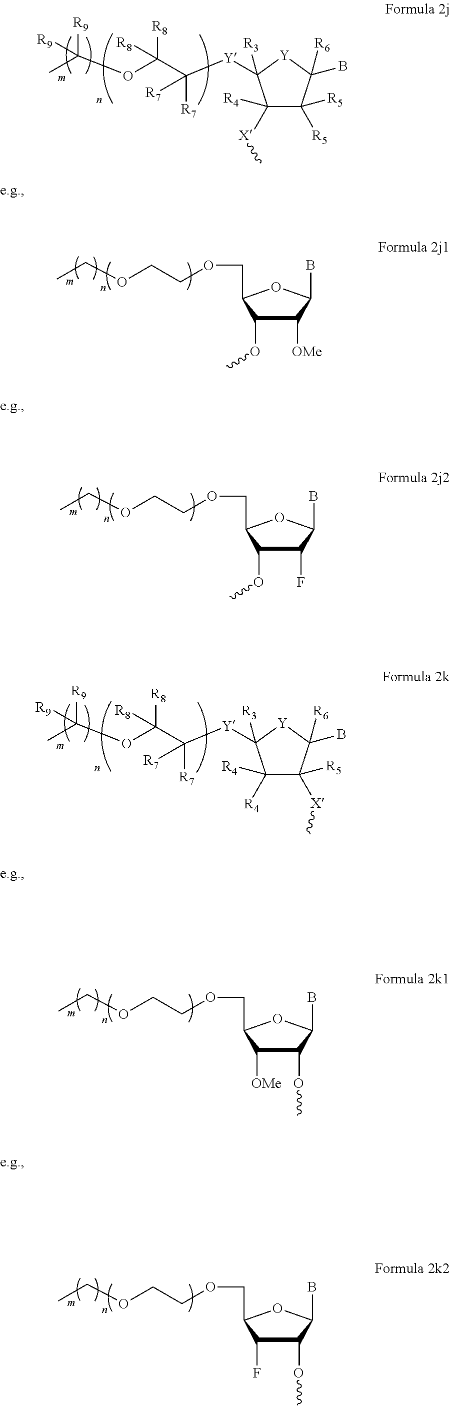

for example:

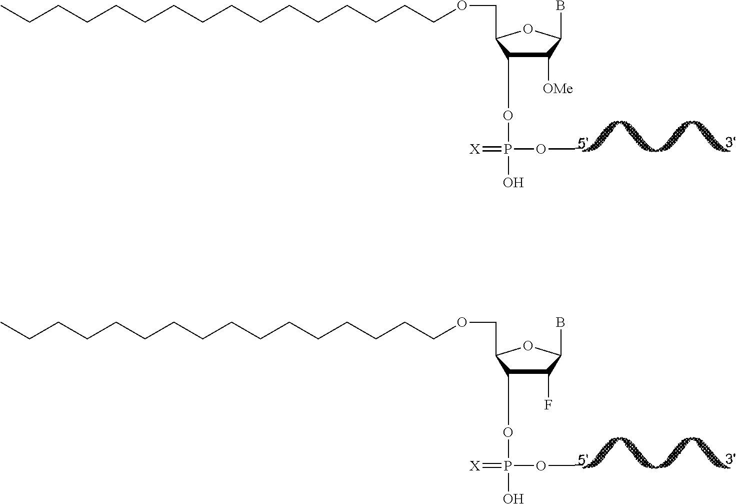

wherein B is a modified or unmodified nucleobase; and the ribbon represents the sense strand; R₃, R₄, R₅, R₆, R₇, R₈, and R₉ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, —CH₂O—, —OCH₂—, or —SS—;
n=0-10;
m=1-30, e.g., 11-21, e.g. 13-17; e.g. 15.
1.46 Any of the preceding compounds, wherein

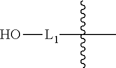

of the sense strand is in accordance with one of the following formulae:

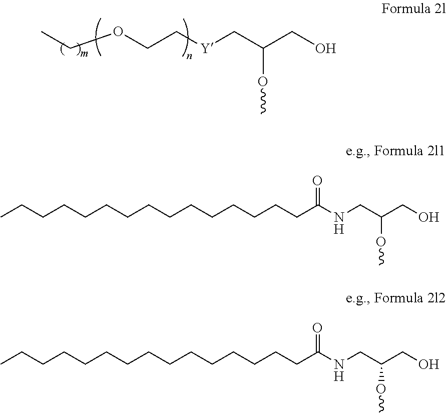

wherein,
Y′ is O, S, CH₂, NH, —NHCO—, —CONH—, or —SS—;
n=0-10;
m=1-30, e.g., 11-21, e.g. 13-17; e.g. 15.
1.47 Any of the preceding compounds, wherein

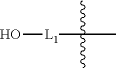

of the sense strand is in accordance with one of the following formulae:

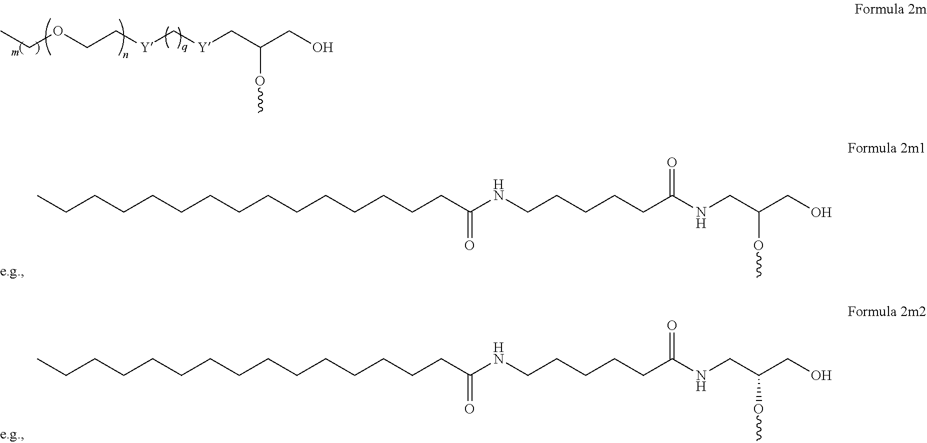

wherein,
Y′ is O, S, CH₂, NH, —NHCO—, —CONH—;
q=1-20;
n=0-10;
m=1-30, e.g., 11-21, e.g. 13-17; e.g. 15.
1.48 Any of the preceding compounds, wherein

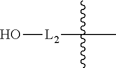

of the sense strand is in accordance with one of the following formulae:

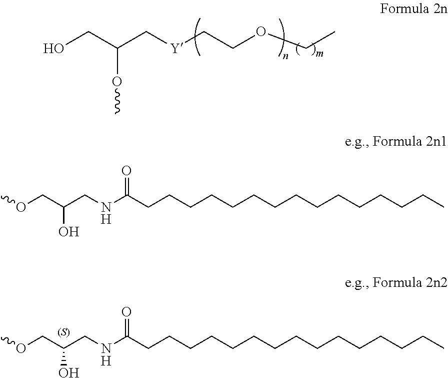

wherein,
Y′ is O, S, CH₂, NH, —NHCO—, or —CONH—;
n=0-10;
m=1-30, e.g., 11-21, e.g. 13-17; e.g. 15.
1.49 Any of the preceding compounds, wherein

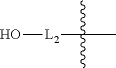

of the sense strand is in accordance with one of the following formulae:

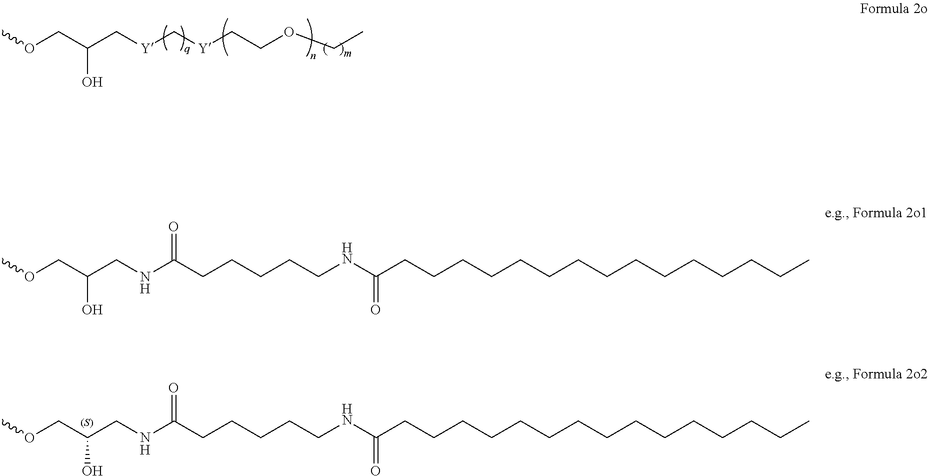

wherein,
Y′ is O, S, CH₂, NH, —NHCO—, —CONH—, or —SS—;
q=1-10;
n=0-10;
m=1-30, e.g., 11-21, e.g. 13-17; e.g. 15.
   for example:

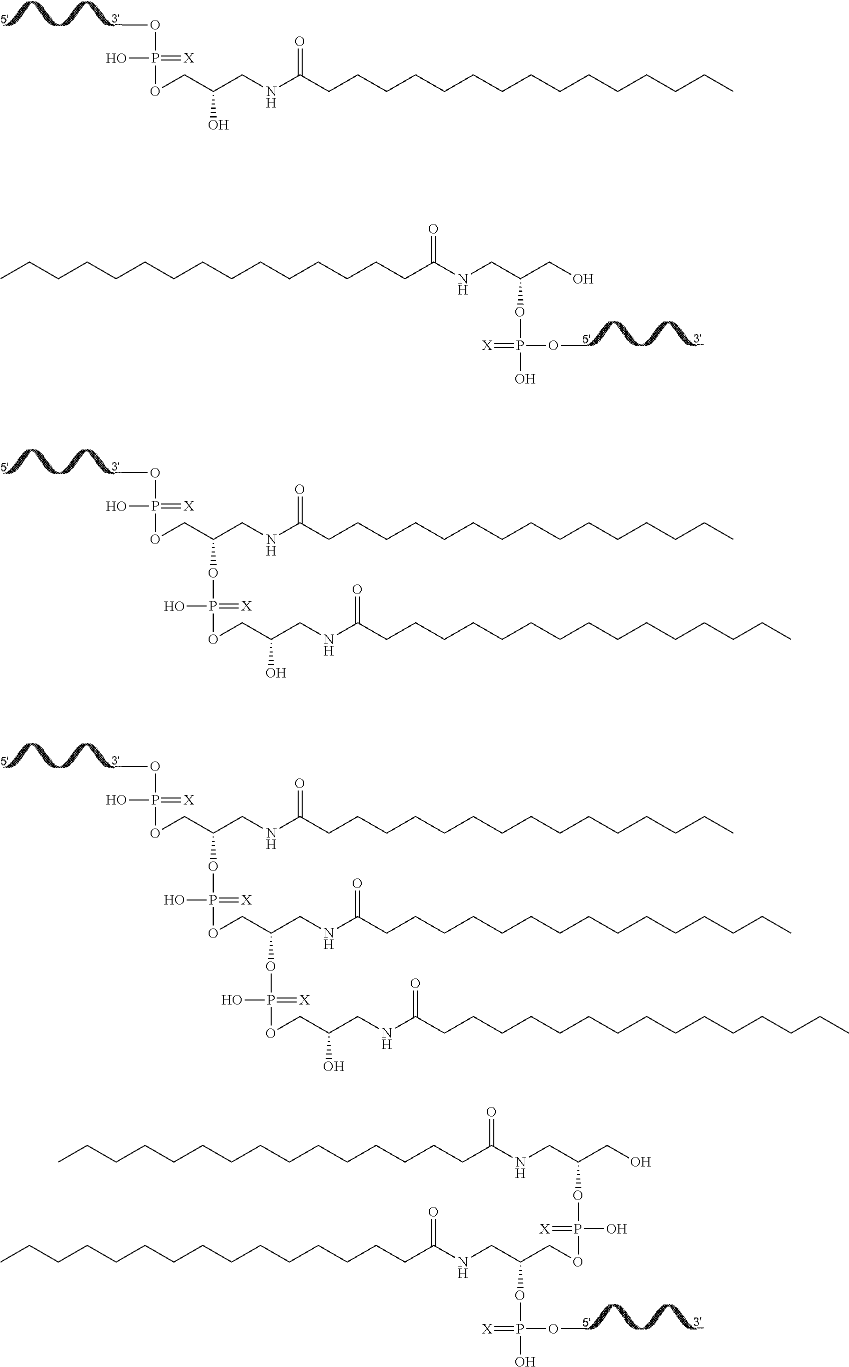

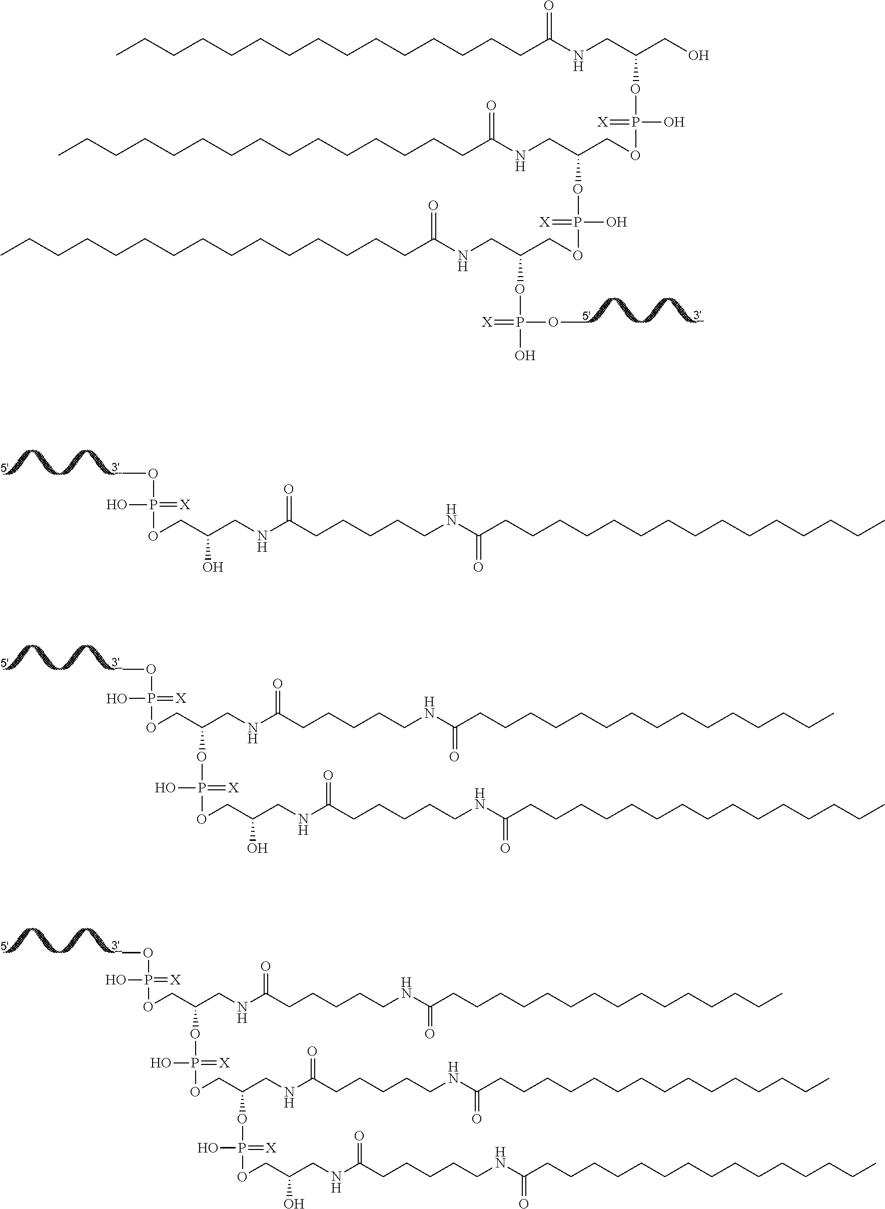

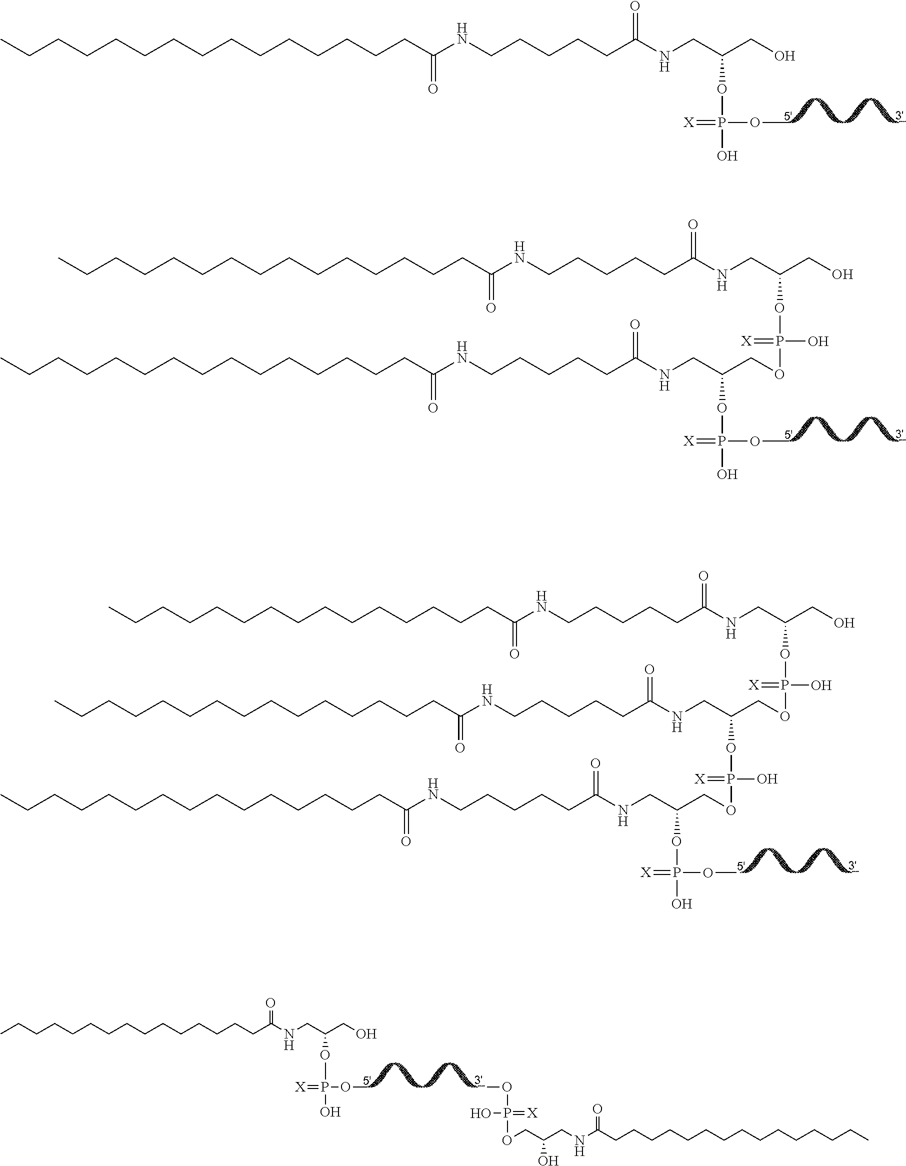

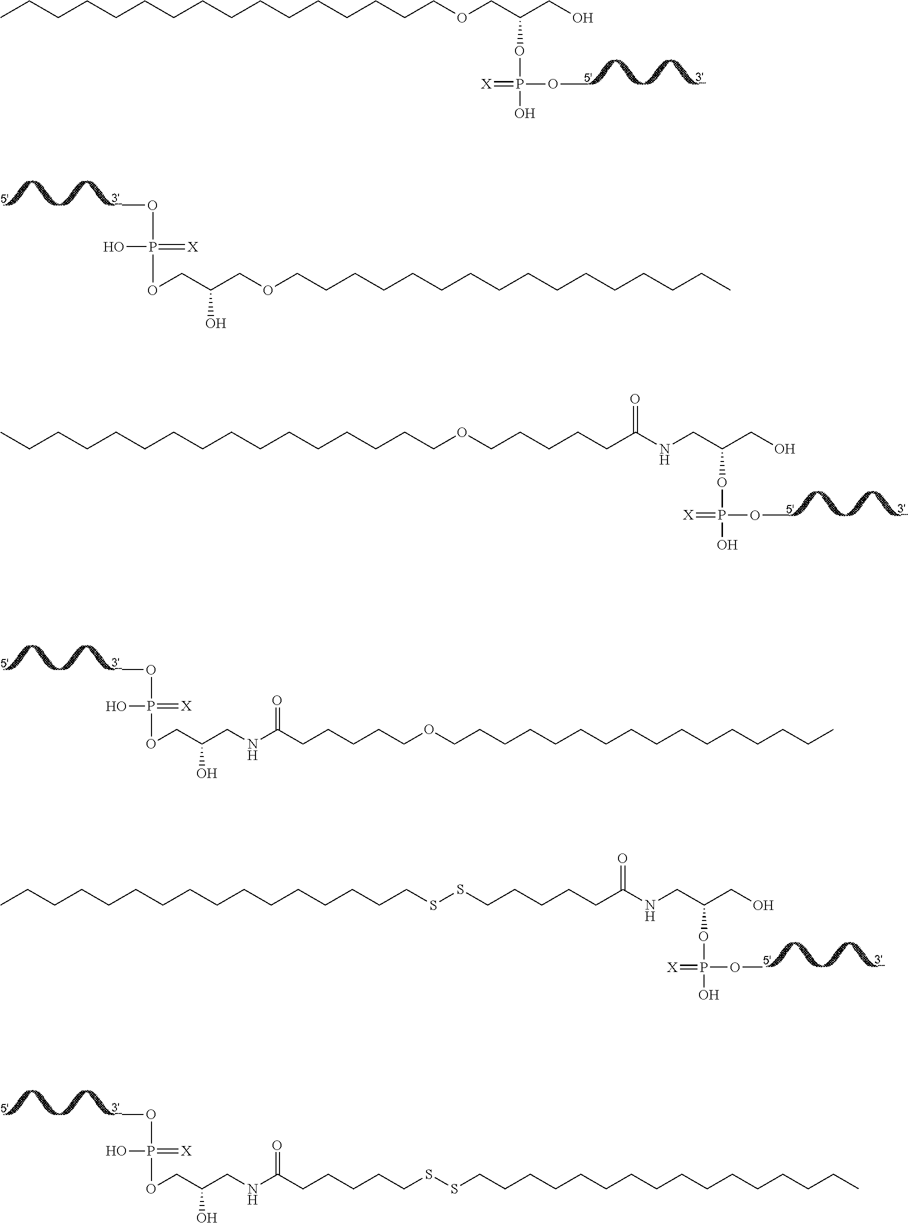

wherein
X═O or S; and the ribbon represents the sense strand,
Y′ is O, S, CH₂, NH, —NHCO—, —CONH—, or —SS—;
q=0-10;
n=0-10;
m=1-30, e.g., 11-21, e.g. 13-17; e.g. 15.
1.50 Any of the preceding compounds, wherein

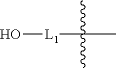

of the sense strand is in accordance with one of the following formulae:

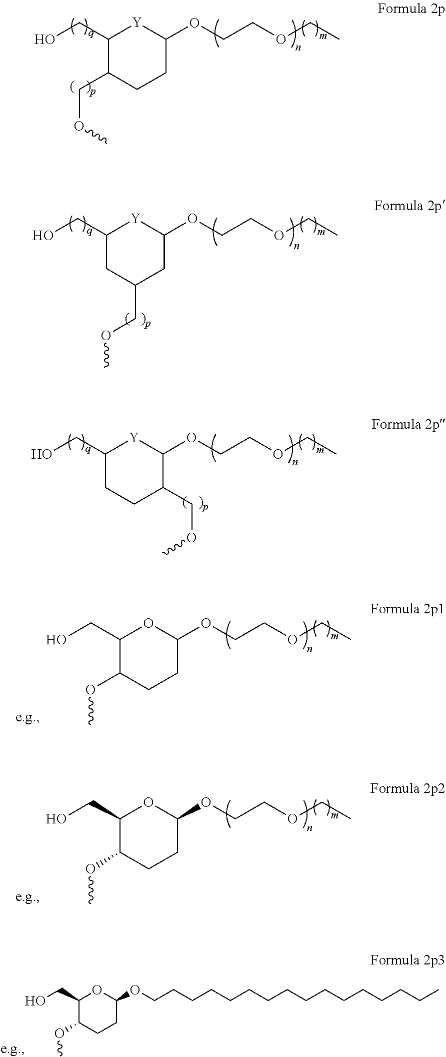

wherein,
Y is O, S, NH or CH₂;
p=0-10,
q=0-10;
n=0-10;
m=1-30, e.g., 11-21, e.g. 13-17; e.g. 15.

1.51 Any of the preceding compounds, wherein

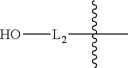

of the sense strand is in accordance with one of the following formulae:

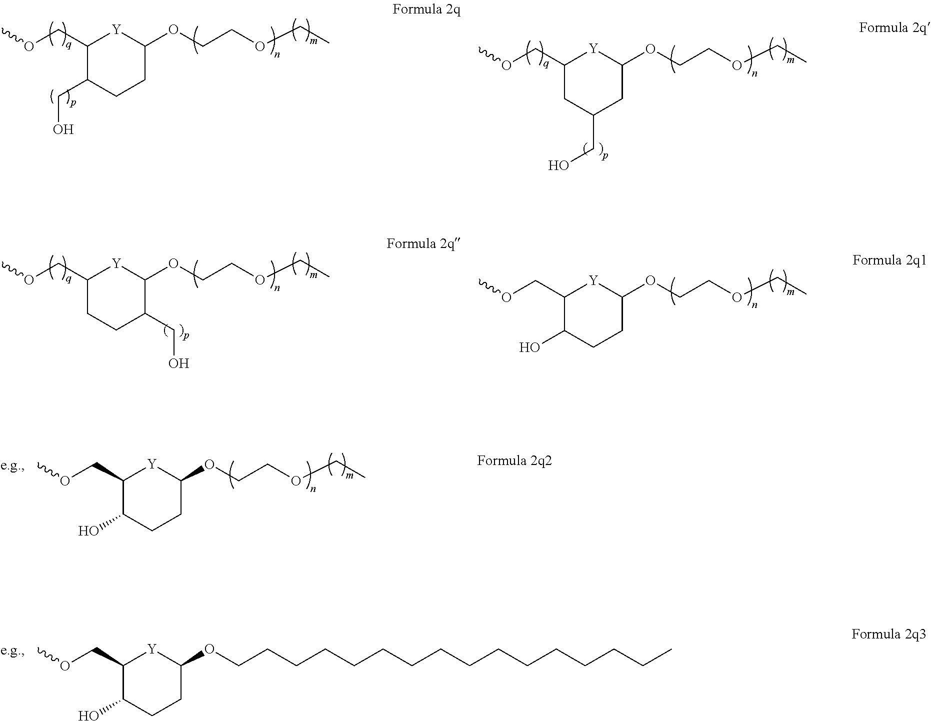

wherein,
Y is O, S, NH or CH₂;
p=0-10,
q=0-10;
n=0-10;
m=1-30, e.g., 11-21, e.g. 13-17; e.g. 15.
1.52 Any of the preceding compounds, wherein

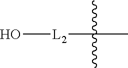

of the sense strand is in accordance with one of the following formulae:

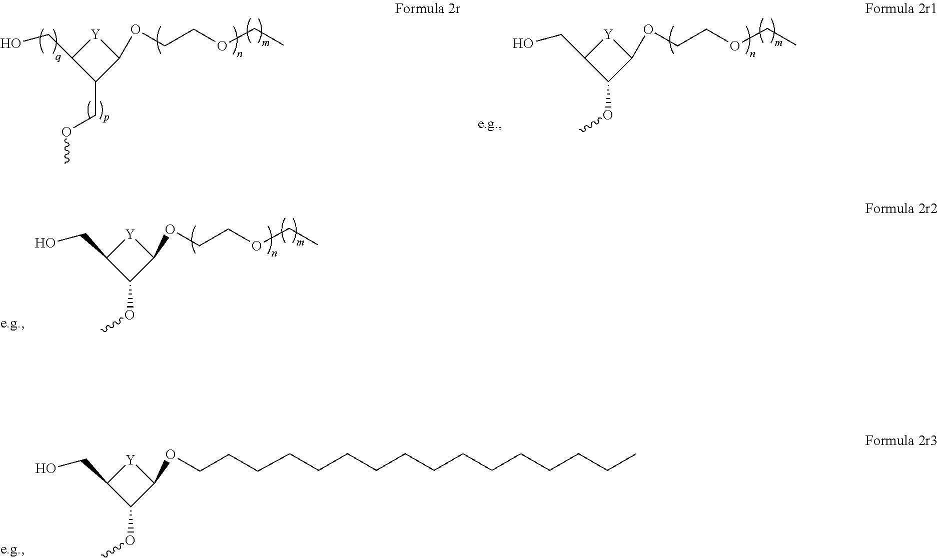

wherein,
Y is O, S, NH or CH₂;
p=0-10, e.g. 0-2;
q=0-10, e.g. 1-2;
n=0-10;
m=1-30, e.g., 11-21, e.g. 13-17; e.g. 15.
1.53 Any of the preceding compounds, wherein

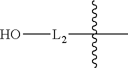

of the sense strand is in accordance with one of the following formulae:

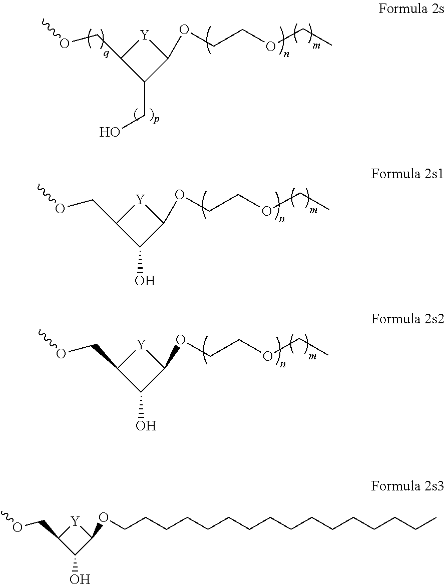

wherein,
Y is O, S, NH or CH₂;
p=0-10, e.g. 0-2;
q=0-10, e.g. 1-2;
n=0-10;
m=1-30, e.g., 11-21, e.g. 13-17; e.g. 15.
1.54 Any of the preceding compounds, wherein the sense strand of the siRNA duplex is modified at one or both ends with one or more lipophilic moieties comprising one or more of the following structure(s):

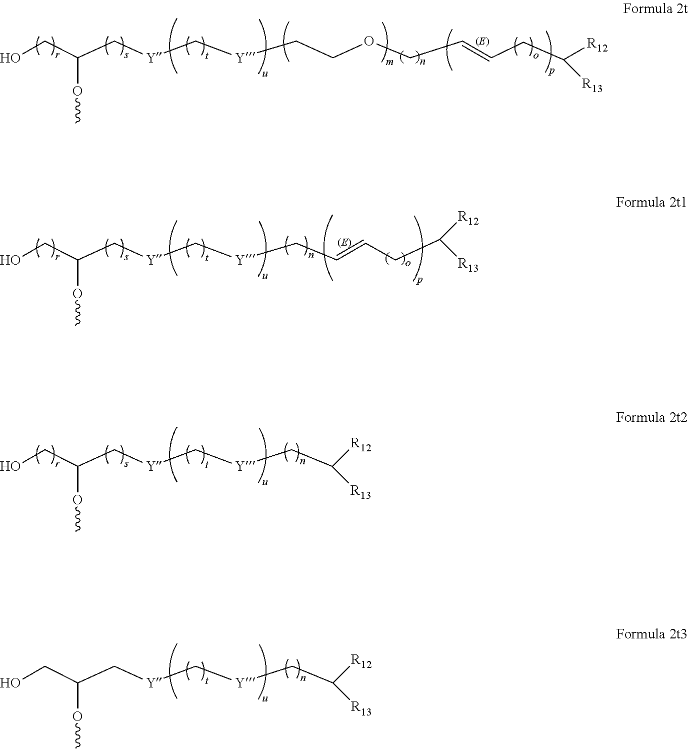

wherein,
R₁₂ and R₁₃ are each independently selected from H, halogen, and C₁-C₆ alkyl, optionally substituted with one or more halogen and/or hydroxy;
Y″ is O, S, CH₂, NH, —NHCO—, —CONH—, or —SS—;
r=1-10, e.g. 1-3;
s=1-10, e.g. 1-3;
t=1-10;
u=0-3;
m=0-5;
n=1-30;
o=1-5.
1.55 Any of the preceding compounds, wherein the sense strand of the siRNA duplex is modified at one or both ends with one or more lipophilic moieties comprising one or more of the following structure(s):

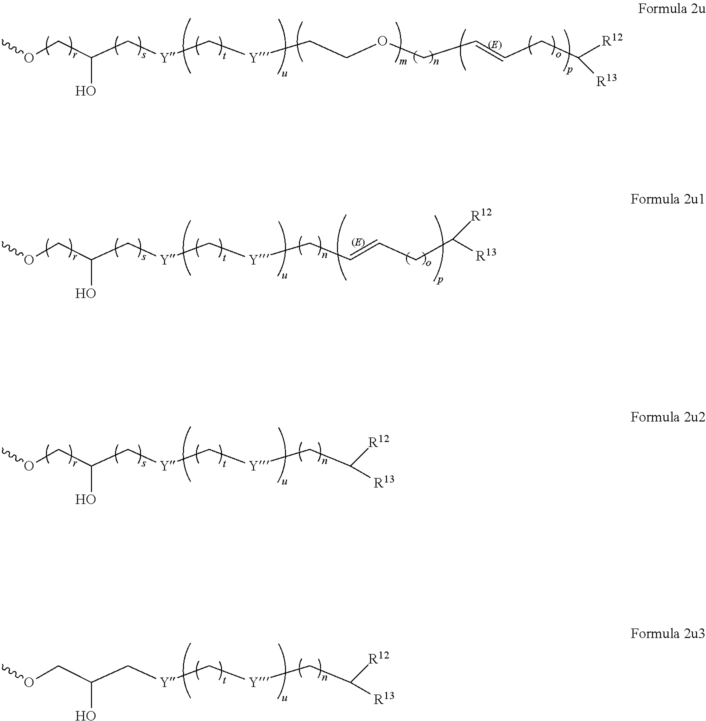

wherein,
R₁₂ and R₁₃ are each independently selected from H, halogen, and C₁-C₆ alkyl, optionally substituted with one or more halogen and/or hydroxy;
Y″ is O, S, CH₂, NH, —NHCO—, —CONH—, or —SS—;
Y′″ is O, S, CH₂, NH, —NHCO—, —CONH, or —SS—;
r=1-10, e.g. 1-3;
s=1-10, e.g. 1-3;
t=1-10;
u=0-3;
m=0-5;
n=1-30;
o=1-5.
1.56 Any of the preceding compounds, wherein the sense strand of the siRNA duplex is modified at one or both ends with one or more lipophilic moieties comprising one or more of the following structure(s):

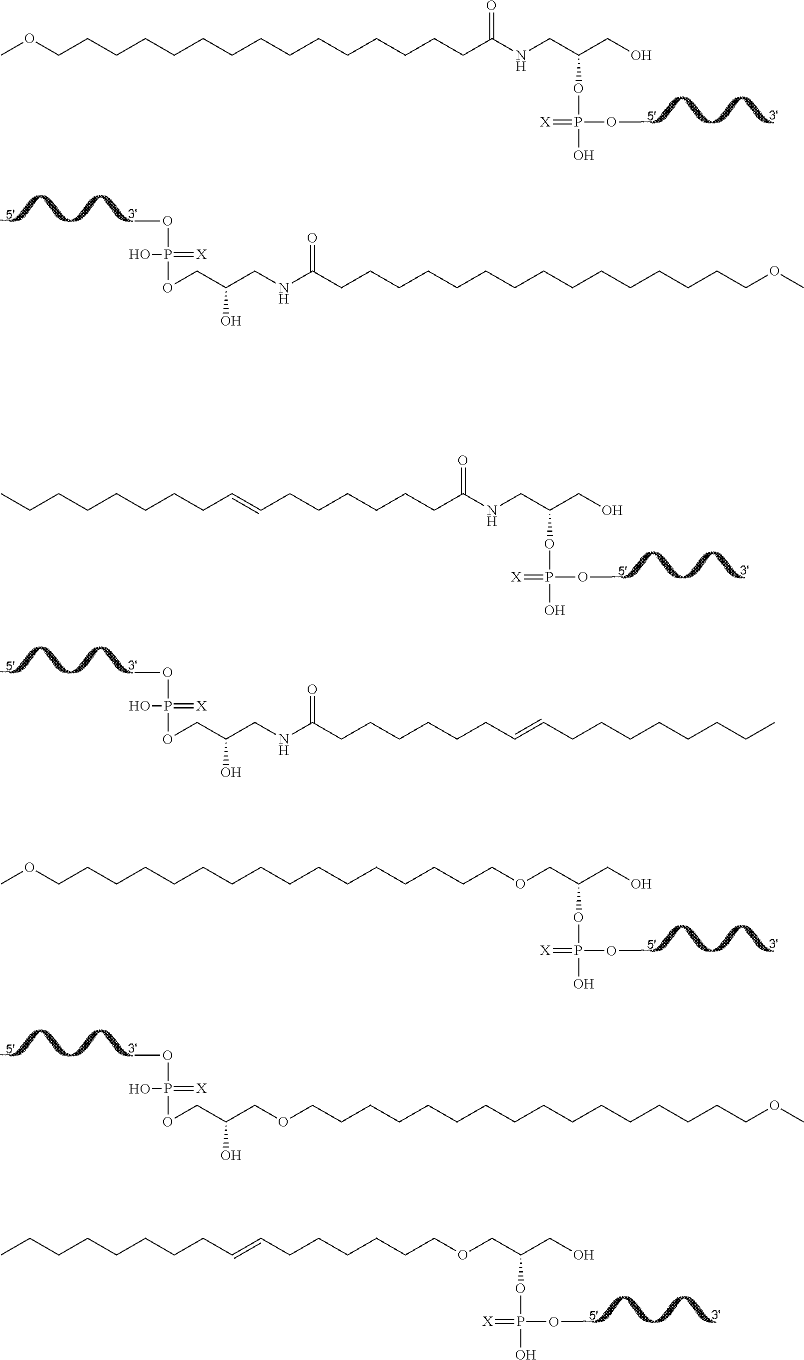

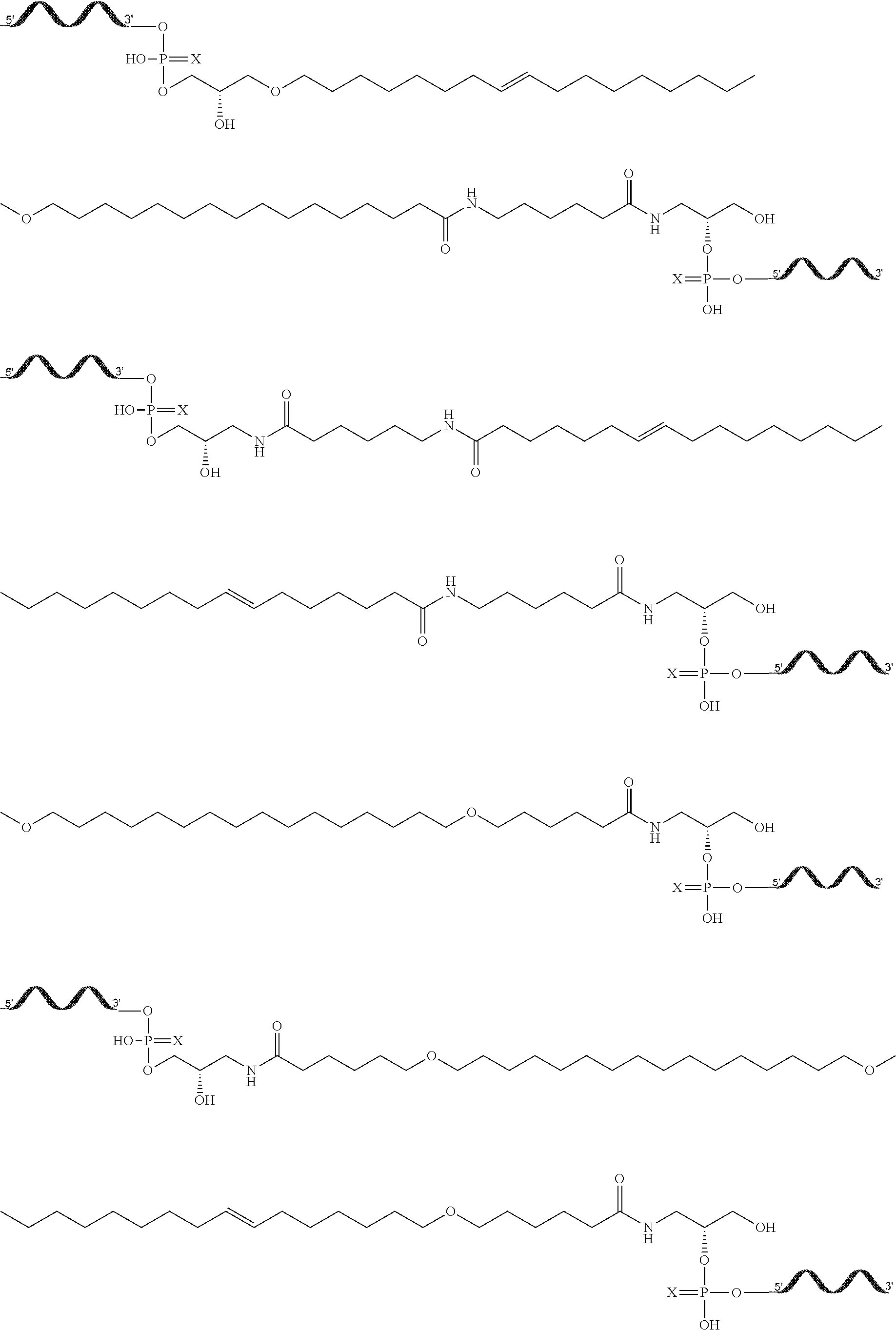

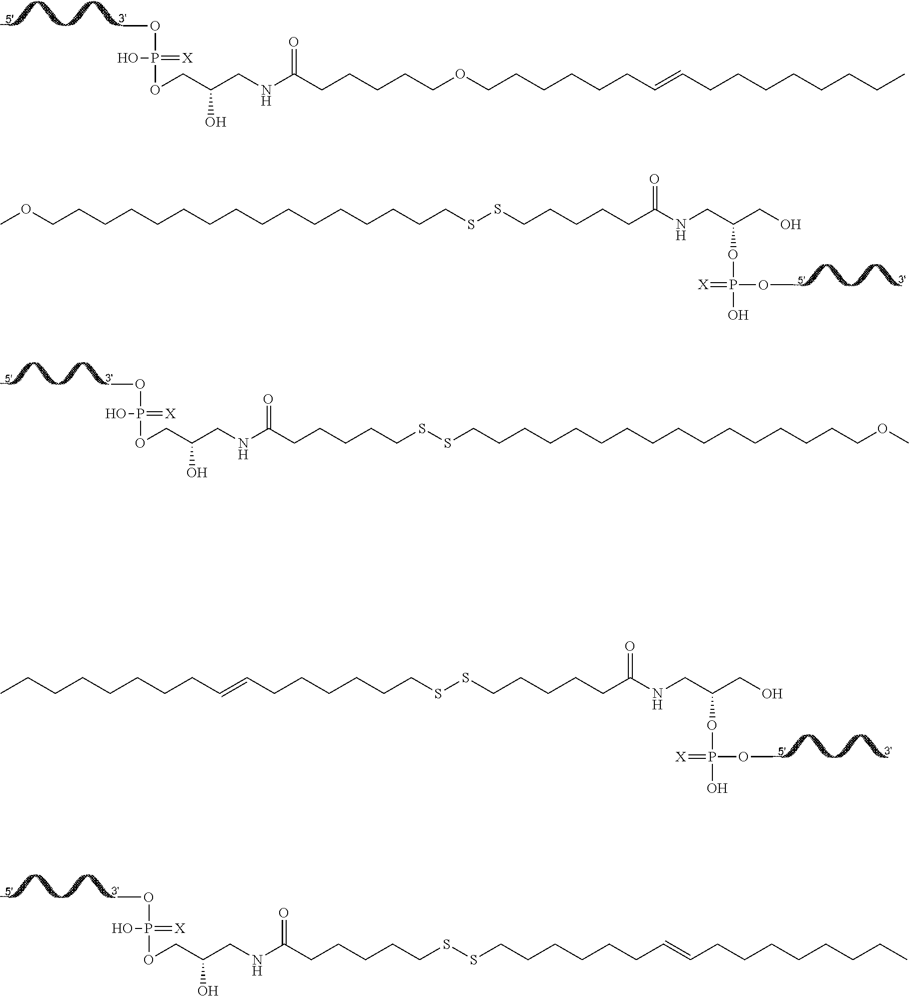

wherein,
X═O or S; and the ribbon represents the sense strand of siRNA or antisense oligonucleotide (ASO).
1.57 Any of the preceding compounds, wherein the sense strand of the siRNA duplex is modified at one or both ends with one or more lipophilic moieties comprising one or more of the following structure(s):

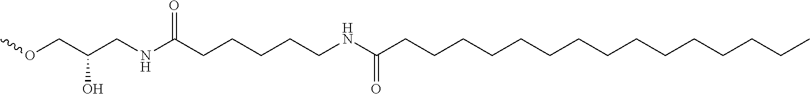

1.58 Any of the preceding compounds, for use in any of Methods 2, et seq., Methods 2A, et. seq., Methods 4, et seq., and/or Methods 6, et seq.

The disclosure further provides a pharmaceutical composition comprising any of Compounds 1, et seq, Compounds 3, et seq., and/or Compounds 5, et seq., in combination or association with a pharmaceutically acceptable diluent or carrier, e.g., wherein the composition is formulated for administration via intravascular, subcutaneous, intramuscular, intraperitoneal, intracerebral, intracerebroventricular, or intrathecal injection, e.g. comprising a delivery vehicle, e.g., a lipid nanoparticle or viral capsid formulation, e.g., for use in any of Methods 2, et seq., Methods 2A, et seq., Methods 4, et seq., and/or Methods 6, et seq.

The disclosure further provides a method (Method 2) of treating a disease or condition characterized by overactivity or overexpression of Lrrk2 (including overactivity or overexpression of an aberrant form of Lrrk2) comprising administering an effective amount of a RNAi agent comprising one or more sequences corresponding to a LRRK2 gene, e.g., a compound selected from any of Compound 1, et seq., to a patient in need thereof.

For example, in particular embodiments the disclosure provides:
2.1. Method 2, wherein the patient is a human.
2.2. Any preceding method, comprising use or administration of any of Compounds 1, et seq., Compounds 3, et seq., and/or Compounds 5, et seq.
2.3. Any preceding method, wherein the patient has been diagnosed with a LRRK2-associated disease, disorder, or pathology, for example inflammatory bowel disease (IBD), Crohn's disease (CD), ulcerative colitis (UC), susceptibility to bacterial infections such as Hansen's disease/leprosy, and/or Parkinson's disease (PD).
2.4. Any preceding method, wherein the disease or condition is selected from inflammatory bowel disease (IBD), Crohn's disease (CD), ulcerative colitis (UC), susceptibility to bacterial infections such as Hansen's disease/leprosy, and/or Parkinson's disease (PD).
2.5. Any preceding method, wherein the disease or condition is selected from Crohn's disease (CD), inflammatory bowel disease (IBD), ulcerative colitis (UC), and Parkinson's disease (PD).
2.6. Any preceding method, wherein the disease or condition is associated with a mutation of LRRK2.
2.7. Any preceding method, wherein the disease or condition is associated with Lrrk2 activity, e.g. enhanced Lrrk2 kinase and/or GTPase activity.
2.8. Any preceding method, wherein the disease or condition is Parkinson's disease or parkinsonism.
2.9. Any preceding method, wherein the disease or condition is associated with a LRRK2 mutation, e.g. selected from mutations associated with Parkinson's Disease, e.g., at one or more of positions 1371, 1437, 1441, 1699, 2019, and/or 2020, e.g. selected from I1371V, N1437H, N1437D, R1441C, R1441G, R1441H, R1441S, Y1699C, G2019S, and I2020T; e.g., G2019S, I2020T, R1441C, R1441G, and R1441H.
2.10. Any preceding method, wherein the disease or condition has symptoms associated with Parkinson's disease, e.g. selected from one or more of loss of motor function, tremors, loss of balance and coordination, stiffness, slowing of movement, ataxia, changes in speech, protein aggregate formation, cognitive decline, neuropathy, and/or neuron death.
2.11. Any preceding method, wherein the compound is administered via intravascular, subcutaneous, intramuscular, intraperitoneal, intracerebral, intracerebroventricular, or intrathecal injection.
2.12. Any preceding method, wherein the compound is administered at about 0.01 mg/kg to about 50 mg/kg.
2.13. Any preceding method, wherein the compound is delivered to the cerebrospinal fluid of the patient via intracerebral, intracerebroventricular, or intrathecal injection, in an effective amount.
2.14. Any preceding method, wherein the compound is delivered to the cerebrospinal fluid of the patient via intracerebral, intracerebroventricular, or intrathecal injection, in an effective amount; e.g., wherein the compound is delivered in an amount effective to achieve a concentration of at least 0.01 nM, e.g., at least 0.1 nM, e.g. at least 1 nM in the cerebrospinal fluid.

The disclosure further provides a method (Method 2A) of treating a disease or condition characterized by or dependent on angiogenesis or lymphangiogenesis comprising administering to a patient in need thereof, an effective amount of a compound which is a double-stranded siRNA duplex, comprising a sense sequence and a complementary antisense sequence, or an antisense oligonucleotide (ASO), wherein the compound comprises one or more sequence(s) corresponding to a region of a VEGFA or ANGPT2 gene, wherein the siRNA duplex or ASO is effective to significantly reduce VEGFA or ANGPT2 mRNA in a cell.

For example, in particular embodiments the disclosure provides:
2A.1. Method 2A, wherein the patient is a human.
2A.2. Any preceding method, comprising use or administration of any of Compounds 1, et seq., Compounds 3, et seq., and/or Compounds 5, et seq.
2A.3. Any preceding method wherein the compound comprises a lipophilic moiety modification, off-target modification, or modification as described in any of the preceding embodiments, e.g., wherein the antisense sequence hybridizes to a sequence selected from SEQ IDS: 201-220, 261-268, 272-285, 356-393, and 394-403; e.g, wherein the antisense sequence comprises a sequence selected in Tables 9-12 and 18-34.
2A.4. Any preceding method wherein the RNAi agent is effective to significantly reduce angiogenesis or lymphangiogenesis.
2A.5. Any preceding method wherein the disease or condition is cancer or ocular neovascular disease.
2A.6. Any preceding method wherein the disease or condition is selected from metastatic colorectal cancer, cervical cancer, endometrial cancer, non-small cell lung cancer, glioblastoma, metastatic hepatocellular carcinoma, metastatic renal cell carcinoma, ovarian cancer, primary peritoneal cancer, and fallopian tube cancer.
2A.7. Any preceding method wherein the disease or condition is selected from neovascular (wet) age-related macular degeneration (AMD), macular edema following retinal vein occlusion, diabetic macular edema, diabetic retinopathy, and myopic choroidal neovascularization.
2A.8. Any preceding method wherein the disease or condition is characterized by overactivity or overexpression of Vegf-A and/or Angpt-2 (including overactivity or overexpression of an aberrant form of Vegf-A and/or Angpt-2).

The disclosure also provides the use of any of Compounds 1, et seq. in the manufacture or making of a medicament for use in any of Methods 2, et seq., Methods 2A, et seq., Methods 4, et seq., and/or Methods 6, et seq.

In another embodiment, the disclosure thus provides RNAi agent comprising a lipophilic moiety, e.g., a lipid moiety, for example an alkyl or alkenyl, e.g. C₁₂₋₂₂ alkyl or alkenyl, at one or both ends of the sense strand or within any position of the sense strand (Compound 3); for example a double-stranded siRNA duplex, comprising a sense sequence and a complementary antisense sequence, wherein the duplex comprises a lipophilic moiety, e.g. a lipid moiety, e.g., alkyl or alkenyl, e.g. C₁₂₋₂₂ alkyl or alkenyl.

For example, in particular embodiments the disclosure provides:
3.1. Compound 3, wherein the lipophilic moiety, e.g., lipid moiety, effectively increases the targeting of the compound to the central nervous system.
3.2. Any preceding compound, comprising one or more of the moieties of Compounds 1, et seq., and/or Compounds 5, et seq.
3.3. Any preceding compound, wherein the compound provides at least 70% mRNA knockdown activity at 10 nM in a cell-based assay.
3.4. Any preceding compound, wherein the compound provides at least 70% mRNA knockdown activity at 1 nM in a cell-based assay.
3.5. Any preceding compound, wherein the compound provides at least 70% mRNA knockdown activity at 0.1 nM in a cell-based assay.
3.6. Any preceding compound, wherein the compound provides at least 70% mRNA knockdown activity at 0.01 nM in a cell-based assay.
3.7. Any preceding compound, wherein the siRNA duplex or ASO comprises a sequence with complementarity to a gene associated with disease.
3.8. Any preceding compound, wherein the siRNA duplex or ASO comprises a sequence with complementarity to a gene associated with a disease of the central nervous system, e.g., Parkinson's disease.
3.9. Any preceding compound, wherein the RNAi agent comprises a sequence with complementarity to LRRK2, e.g.
   a. Any preceding compound, wherein the sense or antisense strand comprises a sequence selected from any one of SEQ ID NOS. 1-403; e.g., a duplex as set forth in Tables 1-34, e.g., Tables 1, 4, 5, 7, 13, 20, 22, 26, and/or 30; e.g., a duplex as set forth in Table 1 other than N3T130025-01.
   b. Any preceding compound, wherein the sense sequence comprises a sequence selected from SEQ ID NOs: 1, 3, 45, 51, 67, 69, 109, 113, 191, and 193.
   c. Any preceding compound, wherein the antisense sequence comprises a sequence selected from SEQ ID NOs: 2, 4, 46, 52, 68, 70, 110, 114, 192, and 194.
   d. Any of the preceding compounds for use in treating a LRRK2-associated disease, disorder, or pathology, for example inflammatory bowel disease (IBD), Crohn's disease (CD), ulcerative colitis (UC), susceptibility to bacterial infections such as Hansen's disease/leprosy, and/or Parkinson's disease (PD).
   e. Any of the preceding compounds for use in treating a disease, disorder, or pathology associated with elevated Lrrk2 activity, e.g., Crohn's disease (CD), inflammatory bowel disease (IBD), ulcerative colitis (UC), and/or Parkinson's disease (PD).
   f. Any of the preceding compounds for use in treating a condition associated with a mutation of LRRK2.
   g. Any of the preceding compounds for use in treating a condition associated with Lrrk2 activity, e.g. enhanced Lrrk2 kinase and/or GTPase activity.
   h. Any of the preceding compounds for use in treating Parkinson's disease or parkinsonism.
   i. Any of the preceding compounds for use in treating a condition associated with a LRRK2 mutation, e.g. selected from mutations associated with Parkinson's Disease, e.g., at one or more of positions 1371, 1437, 1441, 1699, 2019, and/or 2020, e.g. selected from I1371V, N1437H, N1437D, R1441C, R1441G, R1441H, R1441S, Y1699C, G2019S, and I2020T; e.g., G2019S, I2020T, R1441C, R1441G, and R1441H.
   j. Any of the preceding compounds for use in any of Methods 2, et seq.
   k. Any of the preceding compounds for use in any of Methods 4, et seq.
   l. Any of the preceding compounds for use in any of Methods 6, et seq.
3.10. Any preceding compound, wherein the RNAi agent comprises one or more sequence(s) corresponding to a region of a VEGFA gene or an ANGPT2 gene; e.g., any preceding compound, wherein the RNAi agent comprises one or more antisense sequences which hybridize to one or more sequences selected from SEQ IDS: 201-220, 261-268, 272-285, 356-393, and 394-403; e.g, wherein the antisense sequence comprises a sequence selected in Tables 9-12 and 18-34; e.g. for use in any of Method 2A, et seq.
3.11. Any preceding compound wherein the sense and antisense nucleotide sequences have a length of 18-23 nucleotides, wherein the sequences are offset by 1, 2 or 3 nucleotides and/or wherein one strand is 1, 2, or 3 nucleotides longer than the other, so that there is an overhang at one or both ends.
3.12. Any preceding compound which is an siRNA duplex wherein the sense and antisense nucleotide sequences have a length of 15-30 nucleotides, wherein the sequences have a blunt end on one or both ends of the siRNA duplex.
3.13. Any preceding compound wherein the sense strand is 19 nucleotides in length and the antisense strand is 21 nucleotides in length, or the sense strand is 20 nucleotides in length and the antisense strand is 22 nucleotides in length, or the sense strand is 21 nucleotides in length and the antisense strand is 23 nucleotides in length, such that the siRNA duplex has a two-nucleotide overhang, e.g., wherein the overhang extends beyond the 5′ end of the sense sequence, e.g., as depicted on FIG. 1.
3.14. Any of the preceding compounds, one or both sequences have a phosphorylated 5′ end.
3.15. Any of the preceding compounds, wherein the siRNA duplex comprises at least one nucleotide overhang extending beyond the 5′ end of the sense sequence, optionally wherein the overhang comprises two nucleotides.
3.16. Any of the preceding compounds, wherein the siRNA duplex comprises one or both sequences modified with a phosphonate at the 5′ end.
3.17. Any of the preceding compounds, wherein the siRNA duplex comprises one or both sequences modified with off-target modifications, e.g., UNA and/or GNA, optionally wherein the modification is present between nucleotides 2-8 of the antisense sequence when read from 5′ to 3′.
3.18. Any of the preceding compounds, wherein the RNAi agent comprises one or both sequences modified with one or more lipophilic moieties, optionally wherein the one or more lipophilic moieties is conjugated to one or more internal positions in the double stranded region of the siRNA duplex.
3.19. Any of the preceding compounds, wherein the siRNA duplex comprises one or both sequences modified with one or more lipophilic moieties and wherein the one or more lipophilic moieties are conjugated to the siRNA with or without a linker or carrier.
3.20. Any of the preceding compounds, wherein the sense strand of the RNAi agent is modified at one or both ends with one or more lipophilic moieties conjugated to the siRNA with or without a linker or carrier.
3.21. Any of the preceding compounds, wherein the sense strand of the siRNA duplex is modified at one or both ends with one or more lipophilic moieties conjugated to the siRNA with or without a linker or carrier in accordance with any of Compound A, et seq.
3.22. Any of the preceding compounds, wherein the siRNA duplex comprises one or more sequences modified with one or more phosphorothioate linkages between nucleotides.
3.23. Any of the preceding compounds, wherein the siRNA duplex comprises one or more sequences modified with one or more unnatural and/or modified nucleotide, for example 2′-deoxy-2′-fluoro and/or 2′-O-methyl ribosugar modifications.
3.24. Any of the preceding compounds, wherein the siRNA duplex comprises one or more sequences modified with one or more of the modifications described in any of paragraphs [0053]-[0085].
3.25. Any of the preceding compounds, wherein the construct comprises one or more sequence modification selected from the group consisting of a phosphorylated 5′ end, a 3′ end overhang, a phosphonated 5′ end, a UNA and/or GNA motif, a lipophilic moiety, a phosphorothioate linkage, a modified nucleotide such as 2′-deoxy-2′-fluoro and/or 2′-O-methyl ribosugar, and combinations thereof.
3.26. Any of the preceding compounds which is a modified siRNA duplex comprising a sense strand and an antisense strand, wherein the sense strand comprises one or more lipophilic moieties L₁ conjugated to the siRNA via a phosphodiester or phosphorothioate bond (e.g. via a ribosyl moiety linked to a phosphodiester or phosphorothioate bond) to either or both ends of the sense strand, e.g., according to any of Compound A, et seq.
3.27. Any of the preceding compounds, wherein the RNAi agent is encapsulated within a delivery carrier, such as a lipid nanoparticle or modified viral capsid.
3.28. Any of the preceding compounds for use as a pharmaceutical.
3.29. Any of the preceding compounds, comprising one or more of the moieties of Formula 2i (e.g., Formula 2i1 and 212), Formula 2j (e.g., Formula 2j1 and 2j2), Formula 2k (e.g., Formula 2k1 and 2k2), Formula 21 (e.g., 211 and 212), Formula 2m, (e.g., Formula 2 m1 and 2m2), Formula 2n (e.g., 2n1 and 2n2), Formula 20 (e.g., 201 and 202), Formula 2p (e.g., 2p′, 2p″, 2p1, 2p2, and 2p3), Formula 2q (e.g., 2q′, 2q″, 2q1, 2q2, and 2q3), Formula 2r (e.g., Formula 2r1, 2r2, and 2r3), Formula 2s1 (e.g., 2s1, 2s2, and 2s3), Formula 2t (e.g., 2t1, 2t2, and 2t3), and/or Formula 2u (e.g., 2u1, 2u2, and 2u3).
3.30. Any of the preceding compounds, comprising one or more of the following structure(s):

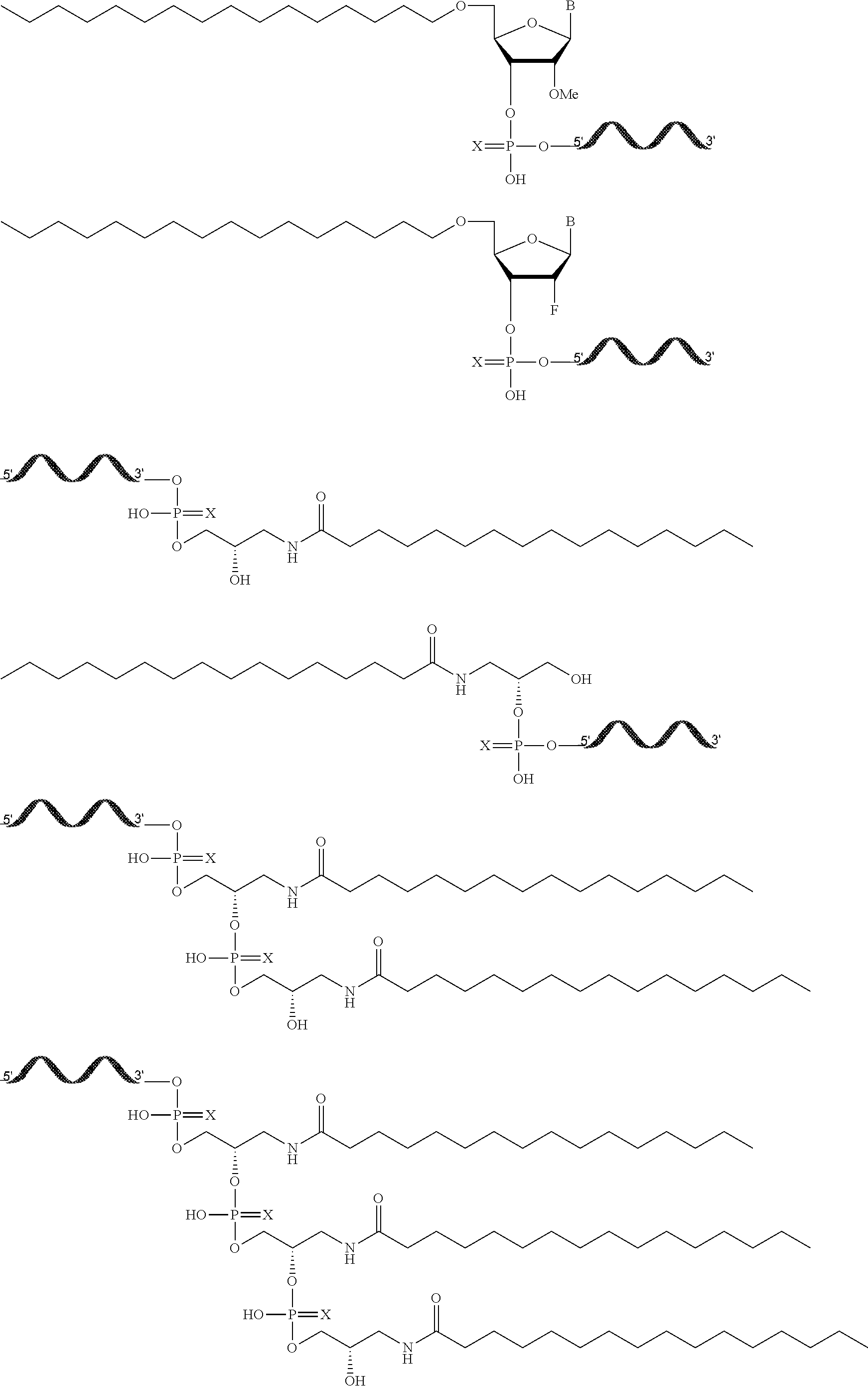

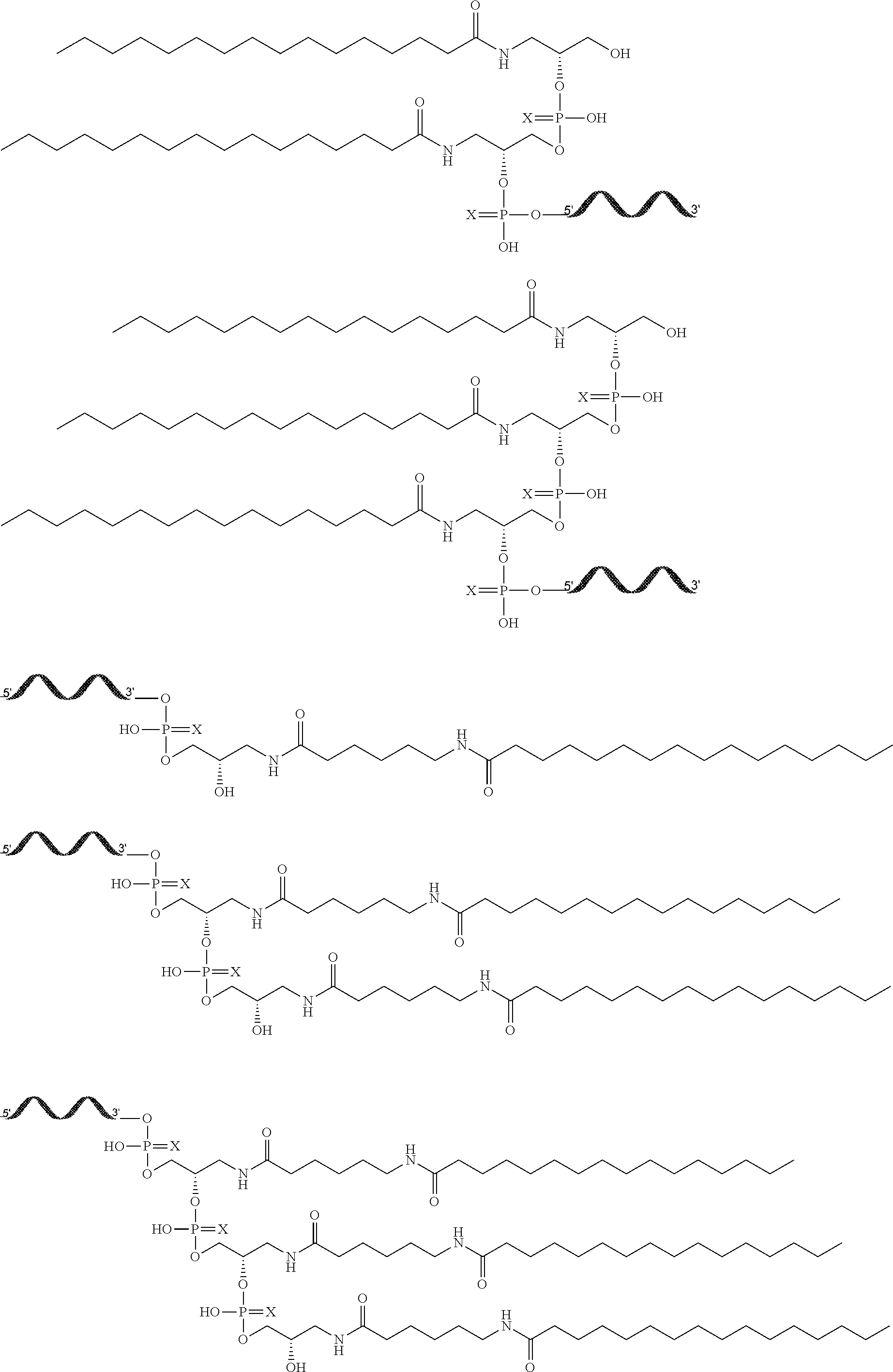

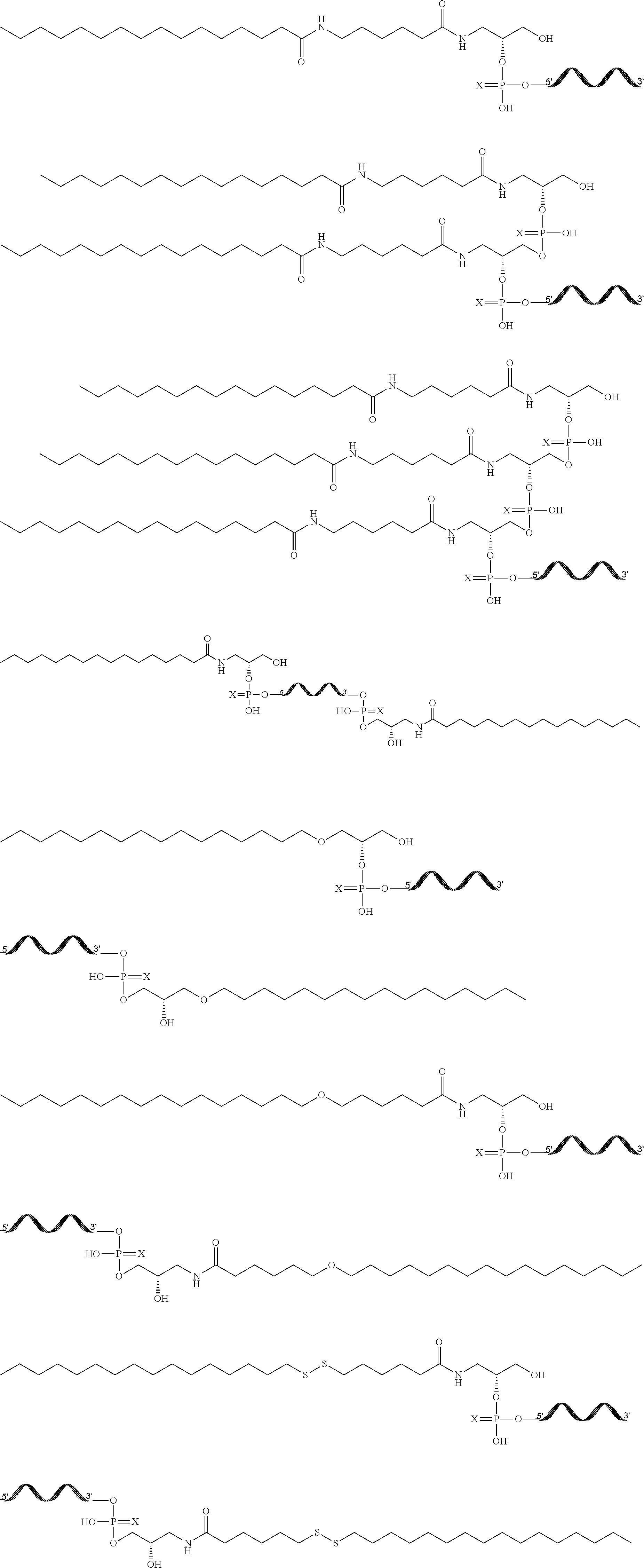

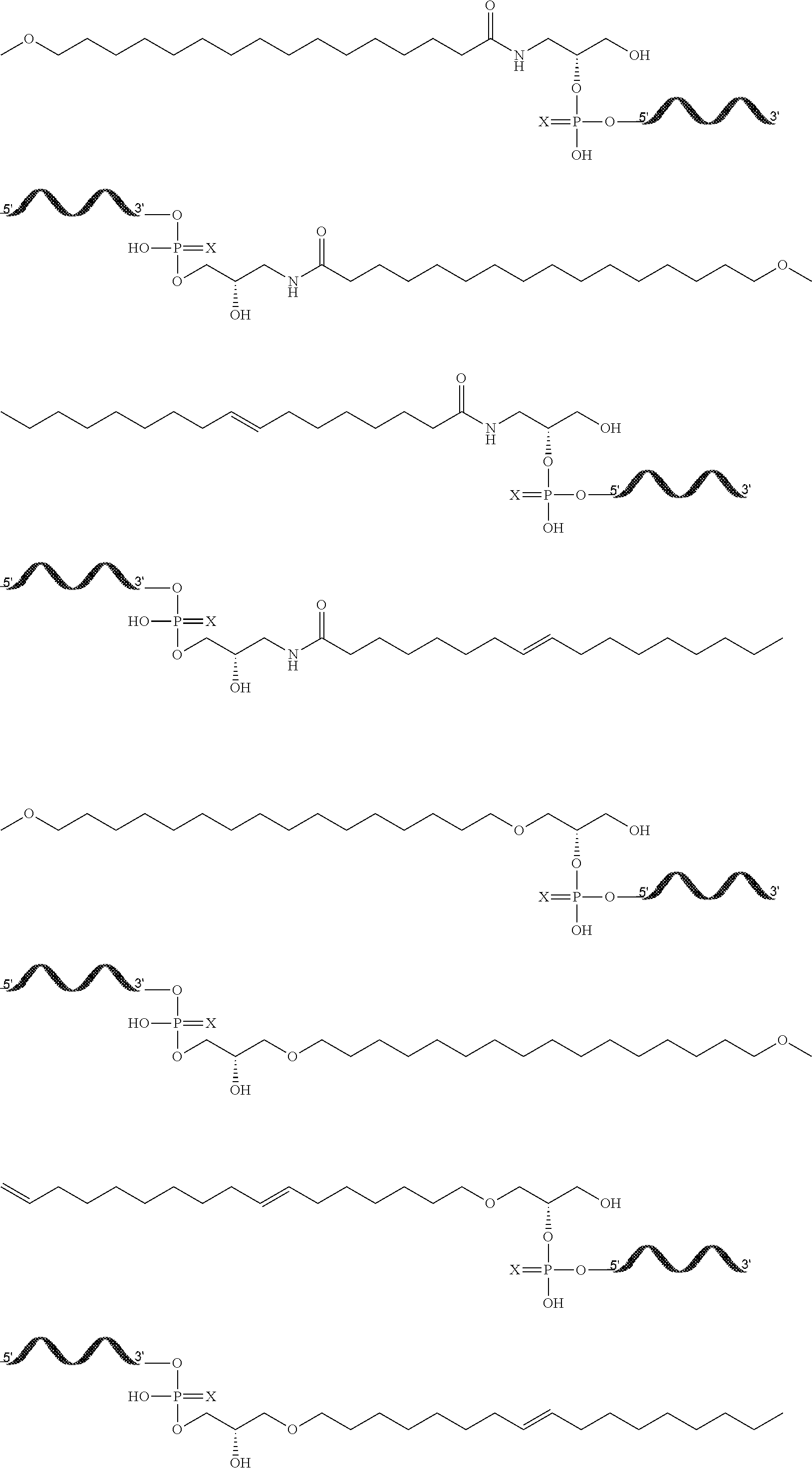

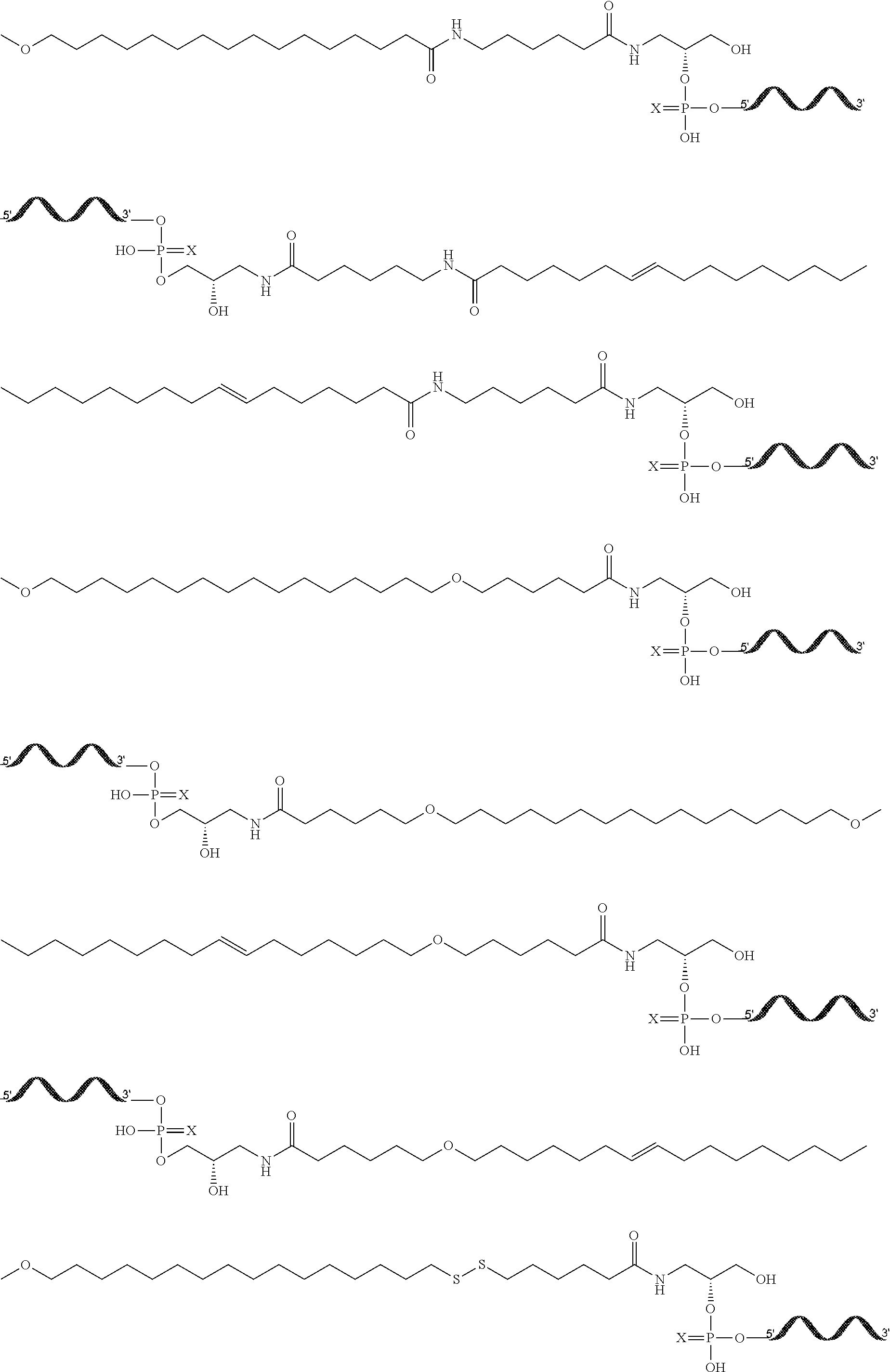

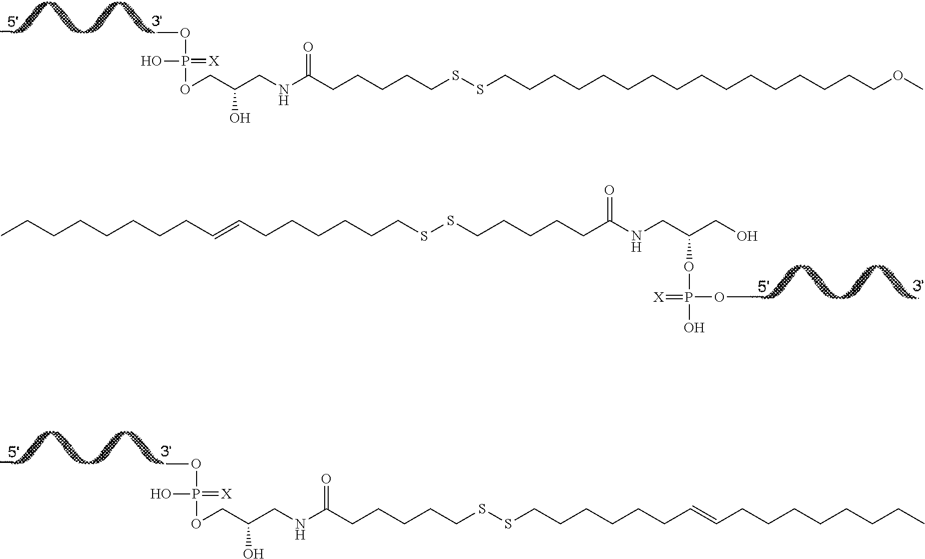

wherein,
X═O or S; and the ribbon represents the sense strand of siRNA or antisense oligonucleotide (ASO).
3.31. Any of the preceding compounds, comprising:

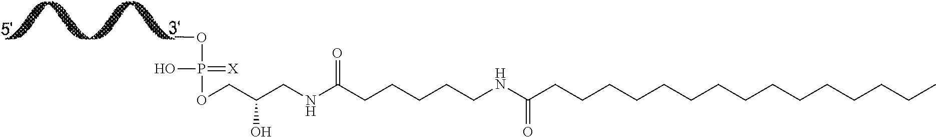

wherein,
X═O or S; and the ribbon represents the sense strand of siRNA or antisense oligonucleotide (ASO).

The disclosure further provides a method (Method 4) of delivering a RNAi agent comprising a lipophilic moiety, e.g., a compound of Compound 3, to a patient in need thereof.

For example, in particular embodiments the disclosure provides:
4.1. Method 4, wherein the patient is a human.
4.2. Any preceding method, comprising delivery of any of Compounds 1, et seq., and/or Compounds 3, et seq., and/or Compounds 5, et seq.
4.3. Any preceding method, wherein the patient has been diagnosed with a LRRK2-associated disease, disorder, or pathology, for example inflammatory bowel disease (IBD), Crohn's disease (CD), ulcerative colitis (UC), susceptibility to bacterial infections such as Hansen's disease/leprosy, and/or Parkinson's disease (PD), e.g., wherein the siRNA or ASO comprise one or more sequences corresponding to a region of a LRRK2 gene.
4.4. Any preceding method, wherein the disease or condition is selected from inflammatory bowel disease (IBD), Crohn's disease (CD), ulcerative colitis (UC), susceptibility to bacterial infections such as Hansen's disease/leprosy, and/or Parkinson's disease (PD).
4.5. Any preceding method, wherein the disease or condition is selected from Crohn's disease (CD), inflammatory bowel disease (IBD), ulcerative colitis (UC), and Parkinson's disease (PD).
4.6. Any preceding method, wherein the disease or condition is associated with a mutation of LRRK2.
4.7. Any preceding method, wherein the disease or condition is associated with Lrrk2 activity, e.g. enhanced Lrrk2 kinase and/or GTPase activity.
4.8. Any preceding method, wherein the disease or condition is Parkinson's disease or parkinsonism.
4.9. Any preceding method, wherein the disease or condition is associated with a LRRK2 mutation, e.g. selected from mutations associated with Parkinson's Disease, e.g., at one or more of positions 1371, 1437, 1441, 1699, 2019, and/or 2020, e.g. selected from I1371V, N1437H, N1437D, R1441C, R1441G, R1441H, R1441S, Y1699C, G2019S, and I2020T; e.g., G2019S, I2020T, R1441C, R1441G, and R1441H.
4.10. Any preceding method, wherein the disease or condition has symptoms associated with Parkinson's disease, e.g. selected from one or more of loss of motor function, tremors, loss of balance and coordination, stiffness, slowing of movement, ataxia, changes in speech, protein aggregate formation, cognitive decline, neuropathy, and/or neuron death.
4.11. Any preceding method, wherein the compound is administered via intravascular, subcutaneous, intramuscular, intraperitoneal, intracerebral, intracerebroventricular, or intrathecal injection.
4.12. Any preceding method, wherein the compound is administered at about 0.01 mg/kg to about 50 mg/kg.
4.13. Any preceding method, wherein the compound is delivered to the cerebrospinal fluid of the patient via intracerebral, intracerebroventricular, or intrathecal injection, in an effective amount.
4.14. Any preceding method, wherein the compound is delivered to the cerebrospinal fluid of the patient via intracerebral, intracerebroventricular, or intrathecal injection, in an effective amount; e.g., wherein the compound is delivered in an amount effective to achieve a concentration of at least 0.01 nM, e.g., at least 0.1 nM, e.g. at least 1 nM in the cerebrospinal fluid.
4.15. Any preceding method wherein the compound is a double-stranded siRNA duplex, comprising a sense sequence and a complementary antisense sequence, or an antisense oligonucleotide (ASO) wherein the sequence(s) correspond to a region of a VEGFA or ANGPT2 gene, wherein the RNAi agent is effective to significantly reduce VEGFA or ANGPT2 mRNA in a cell [e.g., according to any of Compound 1, et seq., Compound 3, et seq., Compound 5, et seq., supra]; e.g. a compound comprising a lipophilic moiety modification, off-target modification, or modification as described in any of the preceding embodiments; e.g., wherein the antisense sequence hybridizes to a sequence selected from SEQ IDS: 201-220, 261-268, 272-285, 356-393, and 394-403; e.g, wherein the antisense sequence comprises a sequence selected in Tables 9-12 and 18-34.
4.16. Any preceding method which is a method of decreasing VEGFA and/or ANGPT2 gene expression in a cell, comprising introducing into the cell a double-stranded siRNA duplex, comprising a sense sequence and a complementary antisense sequence, or an antisense oligonucleotide (ASO) wherein the sequence(s) correspond to a region of a VEGFA or ANGPT2 gene, wherein the siRNA duplex or ASO is effective to significantly reduce VEGFA or ANGPT2 mRNA in a cell; e.g., a method of inhibiting angiogenesis or of treating a disease or condition associated with VEGFA and/or ANGPT2 gene expression, comprising administering an effective amount of a RNAi agent, or pharmaceutical composition thereof; e.g., wherein the disease or condition is cancer or ocular neovascular disease, e.g, wherein the disease or condition is selected from metastatic colorectal cancer, cervical cancer, endometrial cancer, non-small cell lung cancer, glioblastoma, metastatic hepatocellular carcinoma, metastatic renal cell carcinoma, ovarian cancer, primary peritoneal cancer, and fallopian tube cancer, or wherein the disease or condition is selected from neovascular (wet) age-related macular degeneration (AMD), macular edema following retinal vein occlusion, diabetic macular edema, diabetic retinopathy, and myopic choroidal neovascularization; or wherein the disease or condition characterized by overactivity or overexpression of Vegf-A and/or Angpt-2 (including overactivity or overexpression of an aberrant form of Vegf-A and/or Angpt-2), comprising administering an effective amount of a RNAi agent comprising one or more sequences corresponding to a region of a VEGFA or ANGPT2 gene, or pharmaceutical composition thereof, to a patient in need thereof.

The disclosure also provides the use of any of Compounds 3, et seq. in the manufacture or making of a medicament for use in any of Methods 2, et seq, Methods 2A, et seq., Methods 4, et seq., and/or Methods 6, et seq.

In another embodiment, the disclosure thus provides a small interfering ribonucleic acid (siRNA) or antisense oligonucleotide (ASO) comprising one or more off-target modification(s), e.g., a modification effective in reducing off-target effects, at one or both ends of the sense or antisense strand or within any position of the sense or antisense strand (Compound 5).

For example, in particular embodiments the disclosure provides:
5.1. Compound 5, wherein the off-target modification(s) effectively reduces off-target effects or symptoms associated therewith, e.g., side effects of a siRNA therapeutic.
5.2. Any preceding compound, comprising one or more of the moieties of Compounds 1, et seq, and/or Compound 3, et seq.
5.3. Any preceding compound, wherein the compound provides at least 70% mRNA knockdown activity at 10 nM in a cell-based assay.
5.4. Any preceding compound, wherein the compound provides at least 70% mRNA knockdown activity at 1 nM in a cell-based assay.
5.5. Any preceding compound, wherein the compound provides at least 70% mRNA knockdown activity at 0.1 nM in a cell-based assay.
5.6. Any preceding compound, wherein the compound provides at least 70% mRNA knockdown activity at 0.01 nM in a cell-based assay.
5.7. Any preceding compound, which is a siRNA duplex comprising a sequence with complementarity to a gene associated with disease, e.g., a disease of the central nervous system, e.g., Parkinson's disease, e.g., LRRK2.
5.8. Any preceding compound which is a double-stranded siRNA duplex, comprising a sense sequence and a complementary antisense sequence, wherein the duplex comprises an off-target modification, e.g., a modification effective in reducing off-target effects, at one or both ends of the sense or antisense strand or within any position of the sense or antisense strand.
5.9. Any preceding compound which is an siRNA duplex wherein the sense and antisense nucleotide sequences have a length of 15-30 nucleotides, wherein the sequences have a blunt end on one or both ends of the siRNA duplex.
5.10. Any preceding compound, wherein the sense or antisense strand comprises a sequence selected from any one of SEQ ID NOS. 1-403; e.g., a duplex as set forth in Tables 1-34, e.g., Tables 1, 4, 5, 7, 13, 20, 22, 26, and/or 30.; e.g., a duplex as set forth in Table 1 other than N3T130025-01.
5.11. Any preceding compound, wherein the sense or antisense strand comprises a sequence selected from any one of SEQ ID NOS. 261-273 or 314-403
5.12. Any preceding compound, wherein the sense sequence comprises a sequence selected from SEQ ID NOs: 1, 3, 45, 51, 67, 69, 109, 113, 191, and 193.
5.13. Any preceding compound, wherein the antisense sequence comprises a sequence selected from SEQ ID NOs: 2, 4, 46, 52, 68, 70, 110, 114, 192, and 194.
5.14. Any preceding compound wherein the sense and antisense nucleotide sequences have a length of 18-23 nucleotides, wherein the sequences are offset by 1, 2 or 3 nucleotides and/or wherein one strand is 1, 2, or 3 nucleotides longer than the other, so that there is an overhang at one or both ends.
5.15. Any preceding compound wherein the sense strand is 19 nucleotides in length and the antisense strand is 21 nucleotides in length, or the sense strand is 20 nucleotides in length and the antisense strand is 22 nucleotides in length, or the sense strand is 21 nucleotides in length and the antisense strand is 23 nucleotides in length, such that the siRNA duplex has a two-nucleotide overhang, e.g., wherein the overhang extends beyond the 5′ end of the sense sequence, e.g., as depicted on FIG. 1.
5.16. Any of the preceding compounds, one or both sequences have a phosphorylated 5′ end.
5.17. Any of the preceding compounds, wherein the siRNA duplex comprises at least one nucleotide overhang extending beyond the 5′ end of the sense sequence, optionally wherein the overhang comprises two nucleotides.
5.18. Any of the preceding compounds, wherein the siRNA duplex comprises one or both sequences modified with a phosphonate at the 5′ end.
5.19. Any of the preceding compounds, wherein the siRNA duplex comprises one or both sequences modified with off-target modifications, e.g., UNA and/or GNA, optionally wherein the modification is present between nucleotides 2-8 of the antisense sequence when read from 5′ to 3′, e.g., wherein the modification is present between nucleotides 5-7 of the antisense sequence when read from 5′ to 3′, e.g., wherein the modification is present at nucleotide position 5 of the antisense sequence when read from 5′ to 3′, e.g., wherein the modification is present at nucleotide position 7 of the antisense sequence when read from 5′ to 3′.
5.20. Any of the preceding compounds, wherein the siRNA duplex comprises one or both sequences modified with one or more lipophilic moiety, optionally wherein the one or more lipophilic moiety is conjugated to one or more internal positions in the double stranded region of the siRNA duplex.
5.21. Any of the preceding compounds, wherein the siRNA duplex comprises one or both sequences modified with one or more lipophilic moiety and wherein the lipophilic moiety is conjugated to the siRNA with or without a linker or carrier.
5.22. Any of the preceding compounds, wherein the siRNA duplex comprises one or both sequences modified with one or more off-target modifications and wherein the off-target modifications are conjugated to the siRNA with or without a linker or carrier.
5.23. Any of the preceding compounds, wherein the sense and/or antisense strand of the siRNA duplex is modified at one or both ends with one or more lipophilic moieties conjugated to the siRNA with or without a linker or carrier.
5.24. Any of the preceding compounds, wherein the sense and/or antisense strand of the siRNA duplex is modified at one or both ends with one or more off-target modifications conjugations to the siRNA with or without a linker or carrier.
5.25. Any of the preceding compounds, wherein the sense and/or antisense strand of the siRNA duplex is modified at one or both ends with one or more lipophilic moieties conjugated to the siRNA with or without a linker or carrier in accordance with any of Compound A, et seq.
5.26. Any of the preceding compounds, wherein the sense and/or antisense strand of the siRNA duplex is modified at one or both ends with one or more off-target modifications conjugated to the siRNA with or without a linker or carrier in accordance with any of Compound A, et seq.
5.27. Any of the preceding compounds, wherein the siRNA duplex comprises one or more sequences modified with one or more phosphorothioate linkages between nucleotides.
5.28. Any of the preceding compounds, wherein the siRNA duplex comprises one or more sequences modified with one or more unnatural and/or modified nucleotide, for example 2′-deoxy-2′-fluoro and/or 2′-O-methyl ribosugar modifications.
5.29. Any of the preceding compounds, wherein the siRNA duplex comprises one or more sequences modified with one or more of the modifications described in any of paragraphs [0053]-[0085].
5.30. Any of the preceding compounds, wherein the RNAi agent is encapsulated within a delivery carrier, such as a lipid nanoparticle or modified viral capsid.
5.31. Any of the preceding compounds for use as a pharmaceutical.
5.32. Any of the preceding compounds for use in treating a LRRK2-associated disease, disorder, or pathology, for example inflammatory bowel disease (IBD), Crohn's disease (CD), ulcerative colitis (UC), susceptibility to bacterial infections such as Hansen's disease/leprosy, and/or Parkinson's disease (PD).
5.33. Any of the preceding compounds for use in treating a disease, disorder, or pathology associated with elevated Lrrk2 activity, e.g., Crohn's disease (CD), inflammatory bowel disease (IBD), ulcerative colitis (UC), and/or Parkinson's disease (PD).
5.34. Any of the preceding compounds for use in treating a condition associated with a mutation of LRRK2.
5.35. Any of the preceding compounds for use in treating a condition associated with Lrrk2 activity, e.g. enhanced Lrrk2 kinase and/or GTPase activity.
5.36. Any of the preceding compounds for use in treating Parkinson's disease or parkinsonism.
5.37. Any of the preceding compounds for use in treating a condition associated with a LRRK2 mutation, e.g. selected from mutations associated with Parkinson's Disease, e.g., at one or more of positions 1371, 1437, 1441, 1699, 2019, and/or 2020, e.g. selected from I1371V, N1437H, N1437D, R1441C, R1441G, R1441H, R1441S, Y1699C, G2019S, and I2020T; e.g., G2019S, I2020T, R1441C, R1441G, and R1441H.
5.38. Any of the preceding compounds for use in any of Methods 2, et seq.
5.39. Any of the preceding compounds for use in any of Methods 4, et seq.
5.40. Any of the preceding compounds for use in any of Methods 6, et seq.
5.41. Any of the preceding compounds, comprising one or more of the moieties of Tables 20, 22, 24, 26, 28, 30, and/or 32.
5.42. Any preceding compound, wherein the RNAi agent comprises one or more sequence(s) corresponding to a region of a VEGFA gene or an ANGPT2 gene; e.g., any preceding compound, wherein the RNAi agent comprises one or more antisense sequences which hybridize to one or more sequences selected from SEQ IDS: 201-220, 261-268, 272-285, 356-393, and 394-403; e.g, wherein the antisense sequence comprises a sequence selected in Tables 9-12 and 18-34; e.g. for use in any of Method 2A, et seq.

The disclosure further provides a method (Method 6) of delivering a small interfering ribonucleic acid (siRNA) or antisense oligonucleotide (ASO) comprising an off-target modification, e.g., a compound of Compound 5, to a patient in need thereof.

For example, in particular embodiments the disclosure provides:
6.1. Method 6, wherein the patient is a human.
6.2. Any preceding method, comprising delivery of any of Compounds 1, et seq., and/or Compounds 3, et seq., and/or Compounds 5, et seq.
6.3. Any preceding method, wherein the patient has been diagnosed with a LRRK2-associated disease, disorder, or pathology, for example inflammatory bowel disease (IBD), Crohn's disease (CD), ulcerative colitis (UC), susceptibility to bacterial infections such as Hansen's disease/leprosy, and/or Parkinson's disease (PD).
6.4. Any preceding method, wherein the disease or condition is selected from inflammatory bowel disease (IBD), Crohn's disease (CD), ulcerative colitis (UC), susceptibility to bacterial infections such as Hansen's disease/leprosy, and/or Parkinson's disease (PD).
6.5. Any preceding method, wherein the disease or condition is selected from Crohn's disease (CD), inflammatory bowel disease (IBD), ulcerative colitis (UC), and Parkinson's disease (PD).
6.6. Any preceding method, wherein the disease or condition is associated with a mutation of LRRK2.
6.7. Any preceding method, wherein the disease or condition is associated with Lrrk2 activity, e.g. enhanced Lrrk2 kinase and/or GTPase activity.
6.8. Any preceding method, wherein the disease or condition is Parkinson's disease or parkinsonism.
6.9. Any preceding method, wherein the disease or condition is associated with a LRRK2 mutation, e.g. selected from mutations associated with Parkinson's Disease, e.g., at one or more of positions 1371, 1437, 1441, 1699, 2019, and/or 2020, e.g. selected from I1371V, N1437H, N1437D, R1441C, R1441G, R1441H, R1441S, Y1699C, G2019S, and I2020T; e.g., G2019S, I2020T, R1441C, R1441G, and R1441H.
6.10. Any preceding method, wherein the disease or condition has symptoms associated with Parkinson's disease, e.g. selected from one or more of loss of motor function, tremors, loss of balance and coordination, stiffness, slowing of movement, ataxia, changes in speech, protein aggregate formation, cognitive decline, neuropathy, and/or neuron death.
6.11. Any preceding method, wherein the compound is administered via intravascular, subcutaneous, intramuscular, intraperitoneal, intracerebral, intracerebroventricular, or intrathecal injection.
6.12. Any preceding method, wherein the compound is administered at about 0.01 mg/kg to about 50 mg/kg.
6.13. Any preceding method, wherein the compound is delivered to the cerebrospinal fluid of the patient via intracerebral, intracerebroventricular, or intrathecal injection, in an effective amount.
6.14. Any preceding method, wherein the compound is delivered to the cerebrospinal fluid of the patient via intracerebral, intracerebroventricular, or intrathecal injection, in an effective amount; e.g., wherein the compound is delivered in an amount effective to achieve a concentration of at least 0.01 nM, e.g., at least 0.1 nM, e.g. at least 1 nM in the cerebrospinal fluid.
6.15. Any preceding method wherein the compound is a double-stranded siRNA duplex, comprising a sense sequence and a complementary antisense sequence, or an antisense oligonucleotide (ASO) wherein the sequence(s) correspond to a region of a VEGFA or ANGPT2 gene, wherein the siRNA duplex or ASO is effective to significantly reduce VEGFA or ANGPT2 mRNA in a cell [e.g., according to any of Compound 1, et seq., Compound 3, et seq., Compound 5, et seq., supra]; e.g. a compound comprising a lipophilic moiety modification, off-target modification, or modification as described in any of the preceding claims; e.g., wherein the antisense sequence hybridizes to a sequence selected from SEQ IDS: 201-220, 261-268, 272-285, 356-393, and 394-403; e.g, wherein the antisense sequence comprises a sequence selected in Tables 9-12 and 18-34.
6.16. Any preceding method which is a method of decreasing VEGFA and/or ANGPT2 gene expression in a cell, comprising introducing into the cell a double-stranded siRNA duplex, comprising a sense sequence and a complementary antisense sequence, or an antisense oligonucleotide (ASO) wherein the sequence(s) correspond to a region of a VEGFA or ANGPT2 gene, wherein the RNAi agent is effective to significantly reduce VEGFA or ANGPT2 mRNA in a cell; e.g., a method of inhibiting angiogenesis or of treating a disease or condition associated with VEGFA and/or ANGPT2 gene expression, comprising administering an effective amount of a RNAi agent, or pharmaceutical composition thereof; e.g., wherein the disease or condition is cancer or ocular neovascular disease, e.g, wherein the disease or condition is selected from metastatic colorectal cancer, cervical cancer, endometrial cancer, non-small cell lung cancer, glioblastoma, metastatic hepatocellular carcinoma, metastatic renal cell carcinoma, ovarian cancer, primary peritoneal cancer, and fallopian tube cancer, or wherein the disease or condition is selected from neovascular (wet) age-related macular degeneration (AMD), macular edema following retinal vein occlusion, diabetic macular edema, diabetic retinopathy, and myopic choroidal neovascularization; or wherein the disease or condition characterized by overactivity or overexpression of Vegf-A and/or Angpt-2 (including overactivity or overexpression of an aberrant form of Vegf-A and/or Angpt-2), comprising administering an effective amount of a RNAi agent comprising one or more sequences corresponding to a region of a VEGFA or ANGPT2 gene, or pharmaceutical composition thereof, to a patient in need thereof.

The disclosure also provides the use of any of Compounds 5, et seq., in the manufacture or making of a medicament for use in any of Methods 2, et seq, Methods 4, et seq., and/or Methods 6, et seq.

This disclosure further provides compounds, and methods of use thereof, as described supra wherein the RNAi agent comprises a sequence capable of targeting LRRK2, VEGFA, or ANGPT2, e.g., comprising sequences complementary to the target sequences of mRNA produced by a LRRK2, VEGFA, or ANGPT2 gene, e.g., comprising a sequence selected from SEQ ID NO: 201-220, 261-268, 272-285, and 356-403, e.g., comprising a sequence selected from Tables 9-12 or 18-34. In some embodiments, the modifications of siRNA or ASO described supra capable of targeting LRRK2 may alternatively be applied to siRNAs or ASOs targeting VEGFA or ANGPT2. In some embodiments, the RNAi agent comprise one or more modifications as described supra, e.g., comprising any one of the modifications useful for improving the stability, delivery, and/or efficacy of the siRNA duplex or ASO, e.g., comprising any one or more of the lipophilic moieties and/or off-target modifications described supra, while targeting VEGFA or ANGPT2. In some embodiments, such RNAi agent targeting VEGFA or ANGPT2 are useful in methods for treating a disease or condition which may be inhibited by inhibiting angiogenesis mediated by Vegf-A and/or Angpt-2, e.g., for treating cancers, ocular neovascular disorders, and other pathological conditions characterized by or dependent on angiogenesis.

### EXAMPLES

### Example 1. Design and Synthesis of siRNA Duplex and Antisense Oligonucleotide (ASO)

### Bioinformatics

siRNAs targeting human LRRK2 transcript mRNA (NCBI refseqID NG_011709.2; NCBI GeneID: 120892) are designed. The human LRRK2 mRNA (NM_198578.4) is 9239 bases in length. Detailed lists of the unmodified LRRK2 sense and antisense strand nucleotides are shown in Table 1.

Synthesis of siRNA Duplex with or without Chemical Modifications

Unmodified LRRK2 siRNAs are synthesized on an oligonucleotide synthesizer using commercially available 5′-O-DMT-2′-O-(t-butyl-dimethylsilyl)-3′-O-(2-cyanoethyl-N,N-diisopropyl) phosphoramidite, 5′-O-DMT-2′-O-methyl-3′-O-(2-cyanoethyl-N,N-diisopropyl) phosphoramidite, and 5′-O-DMT-2′-fluoro-2′-deoxy-3′-O-(2-cyanoethyl-N,N-diisopropyl) phosphoramidite monomers of uridine (U), 4-N-acetylcytidine (CAc), 6-N-benzoyladenosine (ABz), 2-N-isobutyrylguanosine (GiBu), and a corresponding special solid support following standard protocols for solid phase synthesis and deprotection; optionally, special off-target phosphoramidite monomers following standard protocols for solid phase synthesis and deprotection. To ensure high fidelity of the data, all single strands are purified using high-performance liquid chromatography (HPLC) to >90% purity. The purity and identity of the oligonucleotides are confirmed by reverse-phase HPLC and liquid chromatography-mass spectroscopy (LC-MS), respectively. For annealing, equimolar amounts of the respective single strands are dissolved in water and heated to 90° C. for 10 minutes. After gradual cooling to room temperature, the resulting duplex is lyophilized.

### Synthesis of Antisense Oligonucleotide (ASO)

Synthesis of antisense oligonucleotide (ASO) is carried out on a corresponding Controlled Pore Glass (CPG) solid support on an automated oligonucleotide synthesizer. All reactions are performed on the solid support packed in a column. The antisense oligonucleotide (ASO) comprises a pattern of 2′-O-methoxyethyl and 2′-deoxy nucleotides. All antisense oligonucleotides are HPLC purified to >90% purity. The purity and identity of the oligonucleotides are confirmed by RP-HPLC and LC-MS, respectively. The ASOs are then lyophilized.

TABLE 1
Unmodified Human LRRK2 siRNA Duplexes

| | SEQ | | | SEQ | | |
|---|---|---|---|---|---|---|
| | ID NO | sense strand (5′ to 3′) | Range* | ID NO | antisense strand (5′ to 3′) | Range* |
| N3T130001- | 1 | GUUGGUCACUUCUGUGCAAA | 377 | 2 | UUGCACAGAAGUGACCAACC | 375 |
| 01 | | U | | | C | |
| N3T130002- | 3 | GGGAUUCAGAAAUGUGGAUU | 1807 | 4 | AUCCACAUUUCUGAAUCCCA | 1805 |
| 01 | | | | | G | |
| N3T130003- | 5 | GUUCACCAAUUGAUUCUUAA | 475 | 6 | UAAGAAUCAAUUGGUGAACA | 473 |
| 01 | | A | | | C | |
| N3T130004- | 7 | GACCCUCAGUGGUUUUAGAU | 3347 | 8 | UCUAAAACCACUGAGGGUCC | 3345 |
| 01 | | C | | | A | |
| N3T130005- | 9 | GGGCCAAAUACCUAUGUAAU | 8828 | 10 | UUACAUAGGUAUUUGGCCCU | 8826 |
| 01 | | A | | | G | |
| N3T130006- | 11 | GCUCUUCAAUAUAAAGGCUC | 4437 | 12 | AGCCUUUAUAUUGAAGAGCC | 4435 |
| 01 | | G | | | A | |
| N3T130007- | 13 | GAGCAGCCUAUGAAGGAGAA | 5819 | 14 | UCUCCUUCAUAGGCUGCUCG | 5817 |
| 01 | | G | | | G | |
| N3T130008- | 15 | GAGUGGCAAUGUCAGGUGUU | 864 | 16 | ACACCUGACAUUGCCACUCAU | 862 |
| 01 | | A | | | | |
| N3T130009- | 17 | GCUCACCAUUCCAAUAUCUCA | 5676 | 18 | AGAUAUUGGAAUGGUGAGCC | 5674 |
| 01 | | | | | U | |
| N3T130010- | 19 | GAACUACAUGUCACAGUAUU | 5064 | 20 | AUACUGUGACAUGUAGUUCU | 5062 |
| 01 | | U | | | U | |
| N3T130011- | 21 | GUGCCUCUGUUGAUCGUCUU | 319 | 22 | AGACGAUCAACAGAGGCACA | 317 |
| 01 | | G | | | U | |
| N3T130012- | 23 | GUGCCACAGUUGGCAUAGAU | 4232 | 24 | UCUAUGCCAACUGUGGCACU | 4230 |
| 01 | | G | | | U | |
| N3T130013- | 25 | GUUUUGCAAAAGUAGCUAUG | 2228 | 26 | AUAGCUACUUUUGCAAAACA | 2226 |
| 01 | | G | | | C | |
| N3T130014- | 27 | GGGUUGCUGGAGAUUGAUAU | 5515 | 28 | UAUCAAUCUCCAGCAACCCAG | 5513 |
| 01 | | U | | | | |
| N3T130015- | 29 | GACCUGCCUAGAAAUAUUAU | 5722 | 30 | UAAUAUUUCUAGGCAGGUCA | 5720 |
| 01 | | G | | | G | |
| N3T130016- | 31 | GACUUGCACAGUAAUAAAUU | 3256 | 32 | AUUUAUUACUGUGCAAGUCC | 3254 |
| 01 | | U | | | A | |
| N3T130017- | 33 | GUUGCUCACCAAAUUUGCAA | 2906 | 34 | UGCAAAUUUGGUGAGCAACG | 2904 |
| 01 | | A | | | C | |
| N3T130018- | 35 | GGCCCUGACCAGUAUUCAUUA | 8897 | 36 | AUGAAUACUGGUCAGGGCCA | 8895 |
| 01 | | | | | A | |
| N3T130019- | 37 | GGUCAUCAAUACCGAAGAUG | 6810 | 38 | AUCUUCGGUAUUGAUGACCA | 6808 |
| 01 | | G | | | G | |
| N3T130020- | 39 | GAGCUUCACCAGAAUGCACUC | 3184 | 40 | GUGCAUUCUGGUGAAGCUCC | 3182 |
| 01 | | | | | A | |
| N3T130021- | 41 | GUGGCAAUGUCAGGUGUUAU | 866 | 42 | UAACACCUGACAUUGCCACUC | 864 |
| 01 | | A | | | | |
| N3T130022- | 43 | GCUUGUCCUAAAGUGGAGAG | 3586 | 44 | UCUCCACUUUAGGACAAGCC | 3584 |
| 01 | | U | | | U | |
| N3T130023- | 45 | GGCAAUGUCAGGUGUUAUAA | 868 | 46 | UAUAACACCUGACAUUGCCA | 866 |
| 01 | | U | | | C | |
| N3T130024- | 47 | GGUAGGAUCUGAAGUCUUAG | 5382 | 48 | UAAGACUUCAGAUCCUACCA | 5380 |
| 01 | | A | | | G | |
| N3T130025- | 49 | GGAAAUGUCAGUACUCCAUU | 7015 | 50 | AUGGAGUACUGACAUUUCCU | 7013 |
| 01 | | G | | | A | |
| N3T130026- | 51 | GCCUUAUUUUCCAAUGGGAU | 5226 | 52 | UCCCAUUGGAAAAUAAGGCA | 5224 |
| 01 | | U | | | U | |
| N3T130027- | 53 | GAUUCACUCAGGUCAUCAAA | 3004 | 54 | UUGAUGACCUGAGUGAAUCA | 3002 |
| 01 | | A | | | U | |
| N3T130028- | 55 | GGAACUCUUAUUUAGCCAUA | 3804 | 56 | AUGGCUAAAUAAGAGUUCCC | 3802 |
| 01 | | A | | | U | |
| N3T130029- | 57 | GUUGUCCAAAACACCCUAAGG | 4985 | 58 | UUAGGGUGUUUUGGACAACC | 4983 |
| 01 | | | | | U | |
| N3T130030- | 59 | GCAGAUCCUACAUCAUUCAGA | 8616 | 60 | UGAAUGAUGUAGGAUCUGCA | 8614 |
| 01 | | | | | G | |
| N3T130031- | 61 | GUUUAAGUCUUAUAGGAUAC | 1952 | 62 | UAUCCUAUAAGACUUAAACC | 1950 |
| 01 | | U | | | C | |
| N3T130032- | 63 | GAGAGCGUGUGAUCAGAAUA | 2277 | 64 | AUUCUGAUCACACGCUCUCUC | 2275 |
| 01 | | A | | | | |
| N3T130033- | 65 | GCAGAUGCCAAUCAAGCAAA | 2332 | 66 | UUGCUUGAUUGGCAUCUGCU | 2330 |
| 01 | | G | | | C | |
| N3T130034- | 67 | GCCAUAAUCAGAUCAGCAUCU | 3818 | 68 | AUGCUGAUCUGAUUAUGGCU | 3816 |
| 01 | | | | | A | |
| N3T130035- | 69 | GAUAUGAGCAGCAAUGAUAU | 3739 | 70 | UAUCAUUGCUGCUCAUAUCU | 3737 |
| 01 | | U | | | A | |
| N3T130036- | 71 | GCUGGUCCAAAUCCUGGAGG | 237 | 72 | CUCCAGGAUUUGGACCAGCG | 235 |
| 01 | | A | | | | |
| N3T130037- | 73 | GCUCUCAUUUCUUGACUUAA | 6669 | 74 | UAAGUCAAGAAAUGAGAGCU | 6667 |
| 01 | | A | | | G | |
| N3T130038- | 75 | GACCUUAGAUCUCCUCCUAAC | 546 | 76 | UAGGAGGAGAUCUAAGGUCU | 544 |
| 01 | | | | | U | |
| N3T130039- | 77 | GGUCCUUUAUUUCCAGAUAA | 2590 | 78 | UAUCUGGAAAUAAAGGACCA | 2588 |
| 01 | | G | | | A | |
| N3T130040- | 79 | GAGCUUUCCACAACAGCUAUG | 3207 | 80 | UAGCUGUUGUGGAAAGCUCG | 3205 |
| 01 | | | | | U | |
| N3T130041- | 81 | GGCCUAUCCAAAUAAGAGAC | 4262 | 82 | UCUCUUAUUUGGAUAGGCCA | 4260 |
| 01 | | A | | | G | |
| N3T130042- | 83 | GGACAGCUCUCAUUUCUUGAC | 6664 | 84 | CAAGAAAUGAGAGCUGUCCU | 6662 |
| 01 | | | | | C | |
| N3T130043- | 85 | GCUCUUUGUUCCAGUGUUAU | 9114 | 86 | UAACACUGGAACAAAGAGCU | 9112 |
| 01 | | C | | | C | |
| N3T130044- | 87 | GCCAUGCACUCAUUUCCAGCC | 631 | 88 | CUGGAAAUGAGUGCAUGGCA | 629 |
| 01 | | | | | U | |
| N3T130045- | 89 | GCAUGCCAGAAGAAUCCAGG | 1679 | 90 | CUGGAUUCUUCUGGCAUGCC | 1677 |
| 01 | | G | | | A | |
| N3T130046- | 91 | GAGCAGCUCAUUUUAGAAGG | 3463 | 92 | CUUCUAAAAUGAGCUGCUCC | 3461 |
| 01 | | A | | | A | |
| N3T130047- | 93 | GACCUUUAUUCCUGACUCUUC | 2760 | 94 | AGAGUCAGGAAUAAAGGUCC | 2758 |
| 01 | | | | | A | |
| N3T130048- | 95 | GCACAGGAUUGCACUCCACGU | 6048 | 96 | GUGGAGUGCAAUCCUGUGCU | 6046 |
| 01 | | | | | G | |
| N3T130049- | 97 | GGUGAUCAGAUAUCAGAUGA | 2661 | 98 | CAUCUGAUAUCUGAUCACCA | 2659 |
| 01 | | A | | | U | |
| N3T130050- | 99 | GGGUUUAAGUCUUAUAGGAU | 1950 | 100 | UCCUAUAAGACUUAAACCCA | 1948 |
| 01 | | A | | | G | |
| N3T130051- | 101 | GGAACUCCUGAAUAAGCGAG | 4548 | 102 | UCGCUUAUUCAGGAGUUCCU | 4546 |
| 01 | | G | | | U | |
| N3T130052- | 103 | GGACUGAAGACCUUAGAUCU | 538 | 104 | GAUCUAAGGUCUUCAGUCCA | 536 |
| 01 | | C | | | A | |
| N3T130053- | 105 | GGUAUUAAACGAAGUCCAUG | 993 | 106 | AUGGACUUCGUUUAAUACCA | 991 |
| 01 | | A | | | G | |
| N3T130054- | 107 | GGCAUAGAUGUGAAAGACUG | 4243 | 108 | AGUCUUUCACAUCUAUGCCA | 4241 |
| 01 | | G | | | A | |
| N3T130055- | 109 | GGAUCUGAAGUCUUAGACAA | 5386 | 110 | UGUCUAAGACUUCAGAUCCU | 5384 |
| 01 | | U | | | A | |
| N3T130056- | 111 | GUUGUGGAAGUGUGGGAUAA | 7249 | 112 | UAUCCCACACUUCCACAACAG | 7247 |
| 01 | | G | | | | |
| N3T130057- | 113 | GAUCUGAAGUCUUAGACAAU | 5387 | 114 | UUGUCUAAGACUUCAGAUCC | 5385 |
| 01 | | C | | | U | |
| N3T130058- | 115 | GAGAGAGCGUGUGAUCAGAA | 2275 | 116 | UCUGAUCACACGCUCUCUCUA | 2273 |
| 01 | | U | | | | |
| N3T130059- | 117 | GUGAAUGCCACCGAGGAAUC | 4597 | 118 | AUUCCUCGGUGGCAUUCACA | 4595 |
| 01 | | U | | | A | |
| N3T130060- | 119 | GGUUGUCCAAAACACCCUAAG | 4984 | 120 | UAGGGUGUUUUGGACAACCU | 4982 |
| 01 | | | | | U | |
| N3T130061- | 121 | GCUAGAGAGAGCGUGUGAUC | 2271 | 122 | AUCACACGCUCUCUCUAGCAU | 2269 |
| 01 | | A | | | | |
| N3T130062- | 123 | GGCGCUUCGAGCUAUUUUAC | 1644 | 124 | UAAAAUAGCUCGAAGCGCCU | 1642 |
| 01 | | A | | | C | |
| N3T130063- | 125 | GUGUUCACCAAUUGAUUCUU | 473 | 126 | AGAAUCAAUUGGUGAACACC | 471 |
| 01 | | A | | | A | |
| N3T130064- | 127 | GGUGUUAUAAUAUUGUGGUG | 878 | 128 | ACCACAAUAUUAUAACACCU | 876 |
| 01 | | G | | | G | |
| N3T130065- | 129 | GAAAUUAUCAUCCGACUAUA | 5200 | 130 | AUAGUCGGAUGAUAAUUUCA | 5198 |
| 01 | | U | | | G | |
| N3T130066- | 131 | GUUUGAGUGAAUCCACAAAU | 7040 | 132 | UUUGUGGAUUCACUCAAACA | 7038 |
| 01 | | U | | | C | |
| N3T130067- | 133 | GUGGACCACAUUGAUUCUCUC | 5476 | 134 | GAGAAUCAAUGUGGUCCACA | 5474 |
| 01 | | | | | A | |
| N3T130068- | 135 | GAGGACAGCUCUCAUUUCUU | 6662 | 136 | AGAAAUGAGAGCUGUCCUCU | 6660 |
| 01 | | G | | | G | |
| N3T130069- | 137 | GGGAACUCUUAUUUAGCCAU | 3803 | 138 | UGGCUAAAUAAGAGUUCCCU | 3801 |
| 01 | | A | | | U | |
| N3T130070- | 139 | GGGUUGGUCACUUCUGUGCA | 375 | 140 | GCACAGAAGUGACCAACCCAC | 373 |
| 01 | | A | | | | |
| N3T130071- | 141 | GACGCAGCGAGCAUUGUACCU | 4362 | 142 | GUACAAUGCUCGCUGCGUCA | 4360 |
| 01 | | | | | U | |
| N3T130072- | 143 | GUACUCUCCUGGUCAUCAAUA | 6800 | 144 | UUGAUGACCAGGAGAGUACC | 6798 |
| 01 | | | | | A | |
| N3T130073- | 145 | GCUCCUUUGAAGAUACUAAA | 6991 | 146 | UUAGUAUCUUCAAAGGAGCA | 6989 |
| 01 | | U | | | G | |
| N3T130074- | 147 | GCACUCACGAGCUUUCCACAA | 3199 | 148 | GUGGAAAGCUCGUGAGUGCA | 3197 |
| 01 | | | | | U | |
| N3T130075- | 149 | GAACAAGCCAACUGUUUUCU | 7151 | 150 | GAAAACAGUUGGCUUGUUCU | 7149 |
| 01 | | U | | | U | |
| N3T130076- | 151 | GAAAUACUGGGAGUGGUAAA | 4157 | 152 | UUACCACUCCCAGUAUUUCCC | 4155 |
| 01 | | A | | | | |
| N3T130077- | 153 | CAUCUACACUAGCAAGAAUG | 2642 | 154 | AUUCUUGCUAGUGUAGAUGC | 2640 |
| 01 | | G | | | U | |
| N3T130078- | 155 | GAACUCCUGAAUAAGCGAGG | 4549 | 156 | CUCGCUUAUUCAGGAGUUCC | 4547 |
| 01 | | G | | | U | |
| N3T130079- | 157 | GGACCACAUUGAUUCUCUCAU | 5478 | 158 | GAGAGAAUCAAUGUGGUCCA | 5476 |
| 01 | | | | | C | |
| N3T130080- | 159 | GGACGAGGAAACUCUGAAGA | 168 | 160 | CUUCAGAGUUUCCUCGUCCUC | 166 |
| 01 | | A | | | | |
| N3T130081- | 161 | GCGAGCAUUGUACCUUGCUG | 4368 | 162 | AGCAAGGUACAAUGCUCGCU | 4366 |
| 01 | | U | | | G | |
| N3T130082- | 163 | GUGCUUAGCCUUGGUGCAUC | 6735 | 164 | AUGCACCAAGGCUAAGCACA | 6733 |
| 01 | | U | | | A | |
| N3T130083- | 165 | GGUCAGAGUCAUGAUGACAG | 7497 | 166 | UGUCAUCAUGACUCUGACCG | 7495 |
| 01 | | C | | | A | |
| N3T130084- | 167 | GUCAUAUAACCAGCUGUCUU | 3405 | 168 | AGACAGCUGGUUAUAUGACA | 3403 |
| 01 | | U | | | G | |
| N3T130085- | 169 | GGAGGCCAUAUUUUACUCCU | 7426 | 170 | GGAGUAAAAUAUGGCCUCCU | 7424 |
| 01 | | G | | | C | |
| N3T130086- | 171 | GAGUCUCAGAGGAGCAACUG | 704 | 172 | AGUUGCUCCUCUGAGACUCU | 702 |
| 01 | | A | | | C | |
| N3T130087- | 173 | GGAAGCCUGUUACAAAGCAU | 1185 | 174 | UGCUUUGUAACAGGCUUCCA | 1183 |
| 01 | | U | | | G | |
| N3T130088- | 175 | GAUGCCCUAAGCCAGAAAUGC | 3127 | 176 | AUUUCUGGCUUAGGGCAUCA | 3125 |
| 01 | | | | | A | |
| N3T130089- | 177 | GAUUGGCUGUCUUGAAAAUC | 3924 | 178 | AUUUUCAAGACAGCCAAUCU | 3922 |
| 01 | | U | | | C | |
| N3T130090- | 179 | GACUGGCCUAUCCAAAUAAG | 4258 | 180 | UUAUUUGGAUAGGCCAGUCU | 4256 |
| 01 | | A | | | U | |
| N3T130091- | 181 | GACAUACCCUAGAAAAGAUG | 6839 | 182 | AUCUUUUCUAGGGUAUGUCU | 6837 |
| 01 | | A | | | C | |
| N3T130092- | 183 | GAUGUCUCUCGAAAUGACAU | 3325 | 184 | UGUCAUUUCGAGAGACAUCA | 3323 |
| 01 | | U | | | A | |
| N3T130093- | 185 | GGUCAUUUCUGCUUUAGGAA | 8855 | 186 | UCCUAAAGCAGAAAUGACCU | 8853 |
| 01 | | A | | | C | |
| N3T130094- | 187 | GAGCGUGUGAUCAGAAUAAC | 2279 | 188 | UUAUUCUGAUCACACGCUCU | 2277 |
| 01 | | A | | | C | |
| N3T130095- | 189 | GAUCUCGUGAACAAGAUGUA | 2429 | 190 | ACAUCUUGUUCACGAGAUCC | 2427 |
| 01 | | C | | | A | |
| N3T130096- | 191 | GGACUUGCACAGUAAUAAAU | 3255 | 192 | UUUAUUACUGUGCAAGUCCA | 3253 |
| 01 | | U | | | A | |
| N3T130097- | 193 | GGUCGUGAGGAAUUCUAUAG | 4324 | 194 | UAUAGAAUUCCUCACGACCU | 4322 |
| 01 | | U | | | G | |
| N3T130098- | 195 | GGCCAAAUACCUAUGUAAUA | 8829 | 196 | AUUACAUAGGUAUUUGGCCC | 8827 |
| 01 | | A | | | U | |
| N3T130099- | 197 | GGUCUUUAGAUAUGAGCAGC | 3731 | 198 | CUGCUCAUAUCUAAAGACCG | 3729 |
| 01 | | A | | | C | |
| N3T130100- | 199 | GACGAGGAAACUCUGAAGAA | 169 | 200 | UCUUCAGAGUUUCCUCGUCC | 167 |
| 01 | | G | | | U | |

*Range refers to location found in NM_198578.4.

### Example 2. Synthesis of Monomers

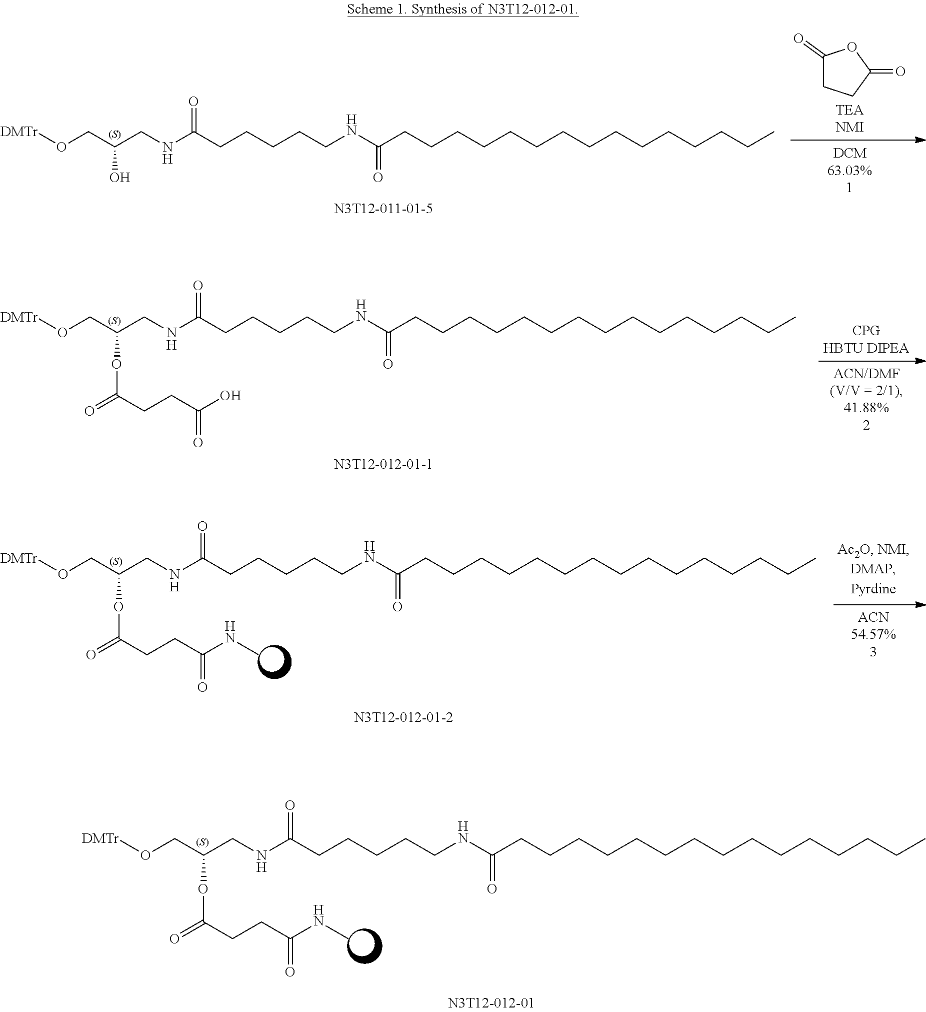

N3T12-012-01-1: To a solution of N3T12-01-01-5 (200 mg, 0.268 mmol) in DCM (2 mL) and N,N-dimethylmethanamide (2 mL) is added NMI (25 mg, 0.295 mmol) and TEA (82 mg, 0.805 mmol). Finally, add tetrahydrofuran-2,5-dione (54 mg, 0.537 mmol). The reaction is stirred for 16 hr at rt. With ice-bath cooling, the reaction is quenched with water and extracted with DCM (200 mL). The organic layer is washed with water and brine, dried over Na₂SO₄ and concentrated to give a residue which is purified by silica gel column chromatography (SiO₂, DCM:MeOH=94:6) to give N3T12-012-01-1 (143 mg, 0.169 mmol, 63.03% yield) as a colorless oil. LCMS: m/z 868.7 [(M+Na)⁺]. ¹H NMR (400 MHz, DMSO-d₆) δ 7.81-7.75 (m, 2H), 7.43-7.35 (m, 4H), 7.25-7.14 (m, 5H), 6.88 (d, J=8.9 Hz, 4H), 5.07-4.94 (m, 1H), 3.74 (s, 6H), 3.21-2.87 (m, 6H), 2.58-2.55 (m, 4H), 2.06-1.91 (m, 4H), 1.41-1.16 (m, 32H), 0.85 (t, J=6.7 Hz, 3H).

N3T12-012-01-2: To a solution of N3T12-012-01-1 (143 mg, 0.169 mmol) in N,N-dimethylmethanamide (3 mL) and ACN (6 mL) is added CPG (500 mg, 0.090 mmol) and DIPEA (19.39 mg, 0.150 mmol), finally,add HBTU (42.67 mg, 0.112 mmol). Shake bed reaction is carried out at 25° C. at a speed of 220 revolutions per minute. The reaction is stirred for 16 h, the filter cake is filtered and rinsed with DCM 3 times, 30 ml each time, acetonitrile 3 times, 30 ml each time, and n-hexane 3 times. The filter cake is dried with a vacuum oil pump for 2 h to give N3T12-012-01-2.

N3T12-012-01: To a solution of Ac₂O (70.44 mg, 0.690 mmol) and Pyridine (109.16 mg, 1.380 mmol),4-(dimethylamino)pyridine (2.53 mg, 0.021 mmol), NMI (1.70 mg, 0.021 mmol) in ACN (9 mL) is added N3T12-012-01-2 (463 mg, 0.069 mmol). Shake bed reaction is carried out at 25° C. at a speed of 220 revolutions per minute. The reaction is stirred for 16 hr at rt under N₂ atmosphere. The filter cake is filtered and rinsed with acetonitrile 3 times, 30 ml each time, The filter cake is dried with a vacuum oil pump for 2 h to give N3T12-012-01 (383 mg, 20.567 μmol, 54.57% yield, loading value=53.7 umol/g).

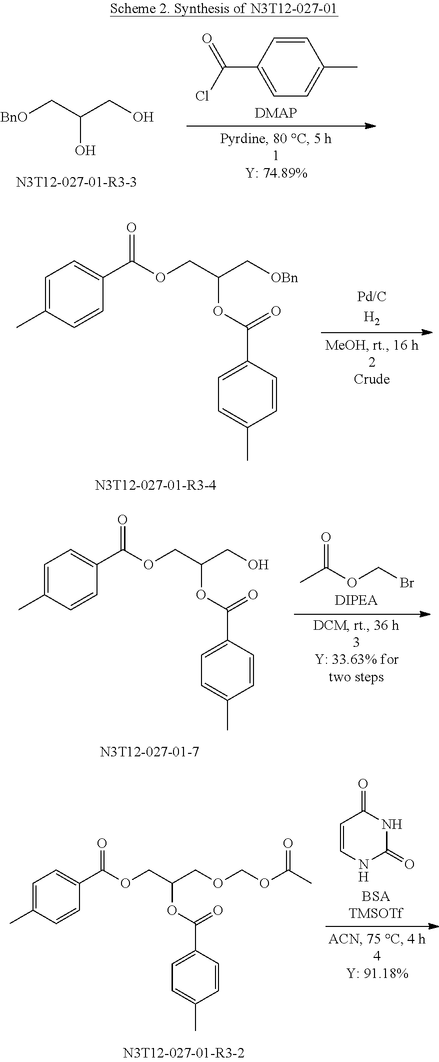

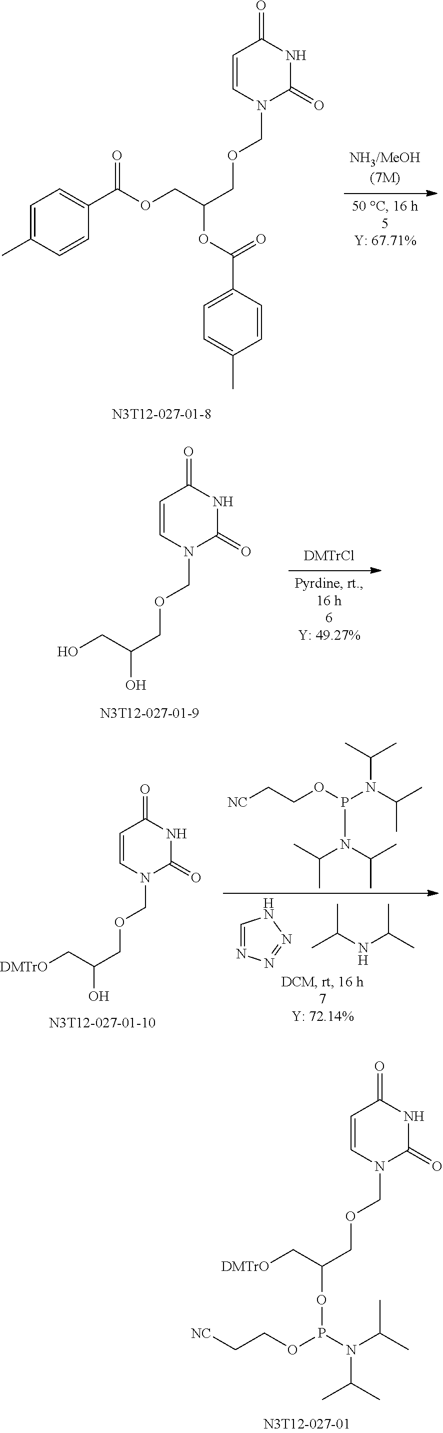

N3T12-027-01-R3-4: To a solution of N3T12-027-01-R3-3 (10.0 g, 54.879 mmol) in pyridine (200 mL) is added 4-methylbenzoyl chloride (12.7 g, 82.318 mmol) and DMAP (6.7 g, 54.879 mmol). The resulting mixture is stirred for 5 h at 80° C. Then, water (50 mL) is added to quench the reaction and evaporated to dryness under reduced pressure. The residue is dissolved in DCM (200 mL) and washed with saturated sodium bicarbonate solution (100 Ml×3). The organic phase is dried with anhydrous sodium sulfate, filterated and evaporated to dryness under reduced pressure. The residue is purified by column chromatography over silica gel (PE/EA=15/1) to give N3T12-027-01-R3-4 (17.2 g, 41.100 mmol, 74.89%) as a colorless oil. LCMS: m/z 441.3 [(M+Na)⁺].

N3T12-027-01-7: To a solution of N3T12-027-01-R₃-4 (12.0 g, 28.675 mmol) in MeOH (80 mL) is added Pd/C (1.2 g, Palladium on activated carbon (5%) (wetted with ca. 55% Water)). The mixture is stirred at room temperature for 16 h under H₂ atmosphere. After the reaction is completed, the solids are filtered off, and the solvent is evaporated to dryness under reduced pressure to afford the N3T12-027-01-7 (10 g, crude) as a colorless oil. The crude is used for the next step without further purification. LCMS: m/z 351.3 [(M+Na)⁺].

N3T12-027-01-R3-2: To a solution of N3T12-027-01-7 (10.0 g, 30.454 mmol) in DCM (100 mL) is added ethyl [di(prop-2-yl)]amine (11.8 g, 91.363 mmol) and bromomethyl acetate (9.3 g, 60.909 mmol). The mixture is stirred at rt for 36 h. After the reaction is completed, the solvent is evaporated to dryness under reduced pressure. The residue is purified by column chromatography (PE/EA=90:10) over silica gel to give N3T12-027-01-R3-2 (4.1 g, 10.239 mmol, 33.63% for two steps) as a colorless oil. LCMS: m/z 423.3 [(M+Na)⁺].

N3T12-027-01-8: To a suspension of N3T12-027-01-R3-2 (1.2 g, 10.713 mmol) in dry ACN is added (1E)-N-(trimethylsilyl)-1-[(trimethylsilyl)oxy]ethanimine (6.7 g, 32.965 mmol). The mixture is heated at 50° C. for 20 min. After cooling to room temperature, a solution of 3-(acetoxymethoxy) propane-1,2-diyl bis(4-methylbenzoate) (3.3 g, 8.241 mmol) in dry ACN along with TMSOTf (6.41 g, 28.844 mmol) is added to the above reaction mixture at 0° C. The solution is stirred for 5 min before heating to 75° C. for 4 h. After the reaction is completed, the solvent is evaporated to dryness under reduced pressure. The residue is purified by column chromatography (DCM/EA=7:3) over silica gel to give N3T12-027-01-8 (3.4 g, 7.514 mmol, 91.18%) as a colorless oil. LCMS: m/z 453.4 [(M+H)⁺].

N3T12-027-01-9: To N3T12-027-01-8 (3.4 g, 7.514 mmol) in a sealed tube is added NH₃ (15 mL, 7 M in MeOH). The resulting mixture is stirred for overnight at 50° C. After the reaction is completed, the solvent is evaporated to dryness under reduced pressure. The residue is purified by column chromatography (DCM/MeOH=95:5) over silica gel to give N3T12-027-01-9 (1.1 g, 5.088 mmol, 67.71%) as a white solid. LCMS: m/z 217.2 [(M+H)⁺].

N3T12-027-01-10: To a solution of N3T12-027-01-9 (1.1 g, 5.088 mmol) in pyridine (50 mL) is added 4,4′-dimethoxytriphenylmethyl chloride (DMTrCl) (2.3 g, 6.615 mmol) in 10 mL THF. The resulting mixture is stirred overnight at room temperature. Then, water (10 mL) is added to quench the reaction and evaporated to dryness under reduced pressure. The residue is purified by column chromatography over silica gel (DCM:EA=5:5) to give N3T12-027-01-10 (1.3 g, 2.507 mmol, 49.27%) as a yellow oil. LCMS: m/z 541.4 [(M+Na)⁺].

N3T12-027-01: To a solution of N3T12-027-01-10 (1.3 g, 2.507 mmol) and di(prop-2-yl) ammonium 1,2,3,4-tetraazol-1-ide (0.64 g, 3.760 mmol) in DCM (6 mL) is added 3-{[2,6-dimethyl-3,5-di(prop-2-yl)-3,5-diaza-4-phosphahept-4-yl]oxy}propanenitrile (1.51 g, 5.014 mmol) in DCM (2 mL). The mixture is stirred at room temperature overnight in the sealed tube. After the reaction is completed, the mixture is poured into a saturated sodium bicarbonate solution (100 mL). Then, the solution is extracted with DCM (100×3). The organic phase is dried with anhydrous sodium sulfate, filtered, and evaporated to dryness under reduced pressure. The residue is purified by reversed-phase column chromatography (0-65% ACN in purified water) to give N3T12-027-01 (1.3 g, 1.809 mmol, 72.14%) as a light yellow solid. LCMS (TM hydrolysate): m/z 658.4 [(M+Na)⁺]. ¹H NMR (400 MHz, DMSO-d₆) δ 11.34 (s, 1H), 7.65 (dd, J=11.0, 7.8 Hz, 1H), 7.41-7.35 (m, 2H), 7.32-7.19 (m, 7H), 6.88 (dt, J=8.8, 4.8 Hz, 4H), 5.59 (dd, J=9.2, 7.8 Hz, 1H), 5.08 (d, J=13.2 Hz, 2H), 4.06-3.94 (m, 1H), 3.81-3.72 (m, 7H), 3.71-3.60 (m, 3H), 3.59-3.43 (m, 2H), 3.12-2.91 (m, 2H), 2.75 (t, J=5.8 Hz, 1H), 2.63 (dd, J=9.2, 3.4 Hz, 1H), 1.17-1.06 (m, 9H), 0.98 (d, J=6.6 Hz, 3H). ³¹P NMR (400 MHz, DMSO-d₆) δ 148.60 (s), 148.17 (s).

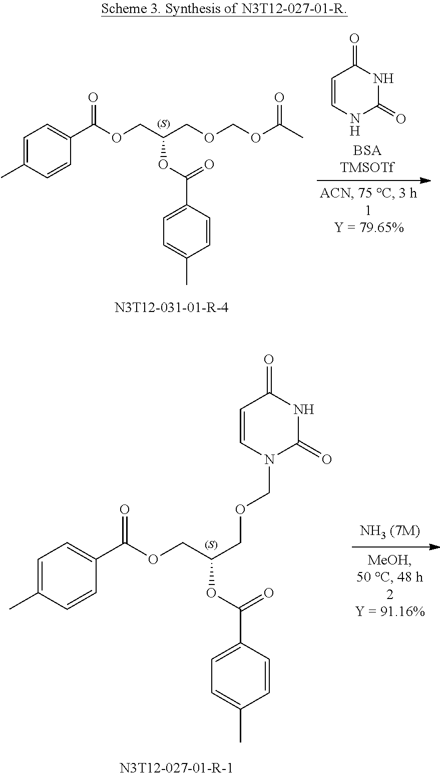

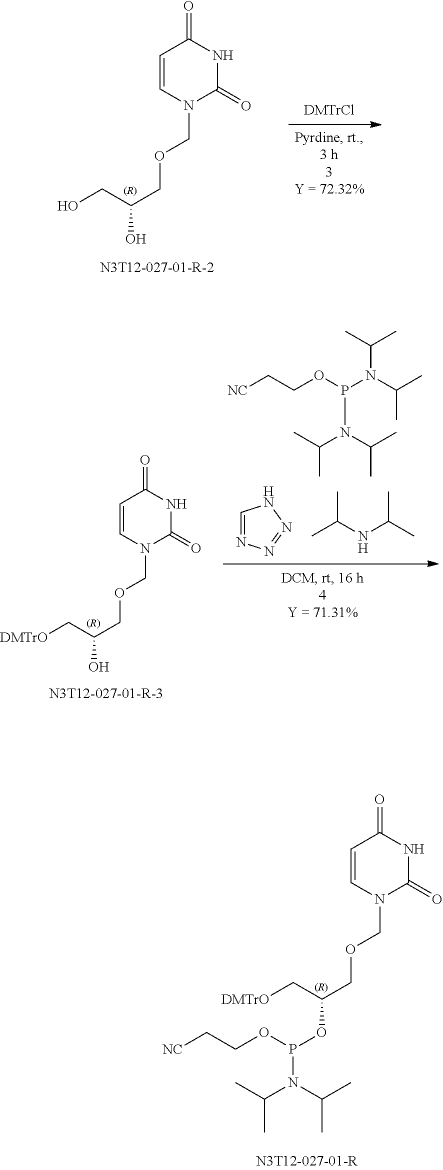

N3T12-027-01-R-1: To a solution of 1,2,3,4-tetrahydropyrimidine-2,4-dione (0.84 g, 7.492 mmol) in ACN (30 mL) is added BSA (4.06 g, 19.979 mmol) at rt under N₂ atmosphere. The reaction is stirred for 30 minutes (mins) at 75° C. Then, N3T12-031-01-R-4 (2.5 g, 6.243 mmol) and TMSOTf (3.75 g, 16.857 mmol) is added at 0° C. The reaction is stirred for 3 hours (h) at 75° C. The reaction is quenched by NaHCO₃(aq, 30 mL) and the mixture is extracted with DCM (3*50 mL). The combined organic layer is dried over Na₂SO₄ and the solvent is removed in vacuo. The residue is purified by flash column chromatography (eluting with 0%-100% EA in PE) to afford N3T12-027-01-R-1 (2.25 g, 4.973 mmol, 79.65%) as a white solid. LCMS: m/z 453.4 [(M+H)⁺]. ¹H NMR (400 MHz, DMSO-d6) δ 11.35 (s, 1H), 7.80 (dd, J=14.0, 8.2 Hz, 4H), 7.71 (d, J=7.8 Hz, 1H), 7.30 (dd, J=8.0, 3.2 Hz, 4H), 5.57 (d, J=7.8 Hz, 1H), 5.51-5.43 (m, 1H), 5.16 (s, 2H), 4.58 (dd, J=11.8, 3.6 Hz, 1H), 4.47 (dd, J=11.8, 6.4 Hz, 1H), 3.91 (d, J=5.0 Hz, 2H), 2.36 (d, J=2.2 Hz, 6H).

N3T12-027-01-R-2: N3T12-027-01-R-1 (2.25 g, 4.973 mmol) is dissolved in ammonia (30 mL, 210 mmol) (7 M in MeOH, 30 mL). The reaction is stirred for 48 hours at 50° C. The solvent is removed in vacuo and the residue is purified by flash column chromatography (eluting with 0%-20% MeOH in DCM) to afford N3T12-027-01-R-2 (0.980 g, 4.533 mmol, 91.16%) as a white solid. ¹H NMR (400 MHz, DMSO-d₆) δ 11.32 (s, 1H), 7.69 (d, J=7.8 Hz, 1H), 5.61 (d, J=7.8 Hz, 1H), 5.13-5.03 (m, 2H), 4.74 (d, J=5.0 Hz, 1H), 4.53 (t, J=5.6 Hz, 1H), 3.56-3.47 (m, 2H), 3.39-3.35 (m, 1H), 3.29 (t, J=5.4 Hz, 2H).

N3T12-027-01-R-3: To a solution of N3T12-027-01-R-2 (980 mg, 4.533 mmol) in Pyrdine (15 mL) is added 1-[chloro(4-methoxyphenyl)phenylmethyl]-4-methoxybenzene (1997 mg, 5.893 mmol) slowly at 0° C. The reaction is stirred for 3 hours at room temperature under N₂ atmosphere. The reaction is quenched by H₂O (20 mL) and the mixture is extracted with DCM (3*30 mL). The combined organic layer is dried over Na₂SO₄ and the solvent is removed in vacuo. The residue is purified by flash column chromatography (eluting with 0%-5% MeOH in DCM) to afford N3T12-027-01-R-3 (1.7 g, 3.278 mmol, 72.32%) as a light-yellow solid. LCMS: m/z 541.1 [(M+Na)⁺]. ¹H NMR (400 MHz, DMSO-d₆) δ 11.35 (s, 1H), 7.65 (d, J=7.8 Hz, 1H), 7.40-7.34 (m, 2H), 7.29 (t, J=7.6 Hz, 2H), 7.24-7.19 (m, 5H), 6.87 (d, J=8.8 Hz, 4H), 5.59 (d, J=7.8 Hz, 1H), 5.08 (s, 2H), 4.95 (d, J=5.4 Hz, 1H), 3.77-3.67 (m, 7H), 3.59 (dd, J=9.8, 4.2 Hz, 1H), 3.49 (dd, J=9.8, 5.8 Hz, 1H), 2.95-2.85 (m, 2H).

N3T12-027-01-R: To a solution of N3T12-027-01-R-3 (700 mg, 1.350 mmol) and Diisopropylammonium Tetrazolide (347 mg, 2.025 mmol) in dry DCM (20 mL) is added 3-{[2,6-dimethyl-3,5-di(prop-2-yl)-3,5-diaza-4-phosphahept-4-yl]oxy}propanenitrile (814 mg, 2.700 mmol) at 0° C. The reaction is stirred for 16 hours at room temperature under N₂ atmosphere. The mixture is extracted with NaHCO₃(aq, 30 mL)/DCM (3*30 mL) and the combined organic layer is dried over Na₂SO₄. The solvent is removed in vacuo and the residue is purifed by reverse-phase column chromatography (eluting with 0%-70% ACN in H2O) to afford N3T12-027-01-R (692 mg, 0.963 mmol, 71.31%) as a light-yellow solid. LCMS: m/z 658.0 [(M-DIPA+OH+Na)⁺]. ¹H NMR (400 MHz, DMSO-d₆) δ 11.37-11.35 (m, 1H), 7.67-7.62 (m, 1H), 7.39-7.36 (m, 2H), 7.32-7.27 (m, 2H), 7.26-7.20 (m, 5H), 6.90-6.82 (m, 4H), 5.65-5.54 (m, 1H), 5.09-5.06 (m, 2H), 4.05-3.94 (m, 1H), 3.79-3.72 (m, 7H), 3.71-3.43 (m, 5H), 3.09-2.92 (m, 2H), 2.78-2.59 (m, 2H), 1.21-1.08 (m, 8H), 0.98 (d, J=6.6 Hz, 4H). ³¹P NMR (162 MHz, DMSO-d6) δ 148.61, 148.18.

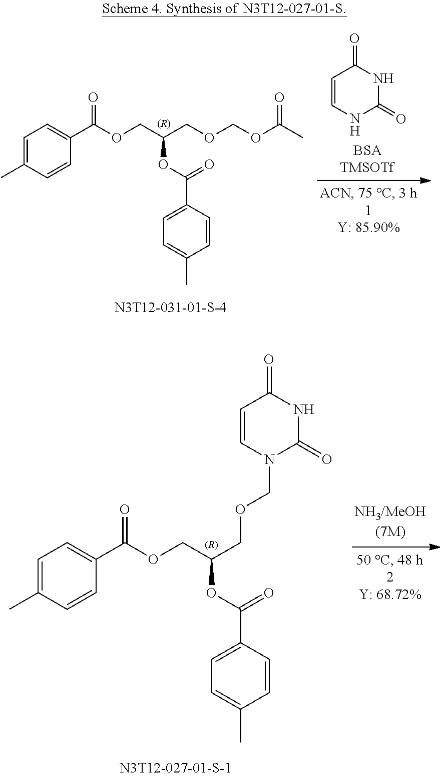

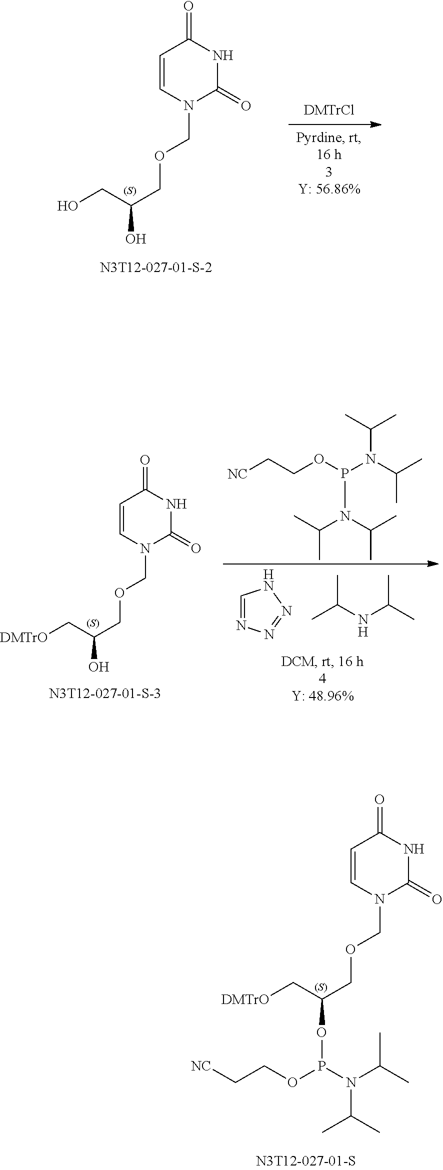

N3T12-027-01-S-1: To a suspension of 1,2,3,4-tetrahydropyrimidine-2,4-dione (1.2 g, 11.038 mmol) in dry ACN (15 mL) is added (1E)-N-(trimethylsilyl)-1-[(trimethylsilyl)oxy]ethanimine (6.9 g, 33.963 mmol). The mixture is heated at 75° C. for 20 minutes under N₂ atmosphere. After cooling to room temperature, a solution of N3T12-031-01-S-4 (3.4 g, 8.491 mmol) in dry ACN (10 mL) along with TMSOTf (6.6 g, 29.718 mmol) are added to above reaction mixture at 0° C. The solution is stirred for 5 minutes before heating to 75° C. for 3 hours. After the reaction is complete, the reaction is quenched by NaHCO₃(aq, 50 mL) and the mixture is extracted with DCM (3*50 mL). The organic phase is dried with anhydrous sodium sulfate, filtrated and evaporated to dryness under reduced pressure. The residue is purified by column chromatography (eluting with 0%-30% EA in DCM) over silica gel to give N3T12-027-01-S-1 (3.3 g, 7.29 mmol, 85.90%) as a colorless oil. LCMS: m/z 453.1 [(M+H)⁺].

N3T12-027-01-S-2: To N3T12-027-01-S-1 (3.3 g, 7.404 mmol) in sealed tube is added NH₃ (20 mL, 7 M in MeOH). The resulting mixture is stirred for 48 hours at 50° C. After the reaction is complete, the solvent is evaporated to dryness under reduced pressure. The residue is purified by column chromatography over silica gel (0-10% MeOH in DCM) to give N3T12-027-01-S-2 (1.1 g, 5.09 mmol, 68.72%) as a white solid. ¹H NMR (400 MHz, DMSO-d₆) δ 11.32 (s, 1H), 7.70 (d, J=7.8 Hz, 1H), 5.61 (d, J=7.8 Hz, 1H), 5.08 (d, J=1.2 Hz, 2H), 4.75 (d, J=5.0 Hz, 1H), 4.54 (t, J=5.6 Hz, 1H), 3.56-3.47 (m, 2H), 3.40-3.35 (m, 1H), 3.29 (t, J=5.4 Hz, 2H).

N3T12-027-01-S-3: To a solution of N3T12-027-01-S-2 (1.1 g, 5.088 mmol) in dry pyridine (40 mL) is added DMTrCl (1.9 g, 5.597 mmol) in 10 mL THF. The resulting mixture is stirred for 16 hours at room temperature under N₂ atmosphere. Then, water (10 mL) is added to quench and the mixture is poured into water (50 mL). Then, the solution is extracted with DCM (50 mL*3). The organic phase is dried with anhydrous sodium sulfate, filtrated and evaporated to dryness under reduced pressure. The residue is purified by column chromatography over silica gel (0-3% MeOH in DCM) to give N3T12-027-01-S-3 (1.5 g, 2.89 mmol, 56.85%) as a yellow oil. LCMS: m/z 541.1 [(M+Na)⁺].

N3T12-027-01-S: N3T12-027-01-S-3 (900 mg, 1.736 mmol) and di(prop-2-yl) ammonium 1,2,3,4-tetraazol-1-ide (445 mg, 2.603 mmol) in DCM (20 mL) is added 3-{[2,6-dimethyl-3,5-di(prop-2-yl)-3,5-diaza-4-phosphahept-4-yl]oxy}propanenitrile (1046 mg, 3.471 mmol). The mixture is stirred at room temperature for 16 hours in a sealed tube under N₂ atmosphere. After the reaction is complete, the mixture is poured into a saturated sodium bicarbonate solution (100 mL). Then, the solution is extracted with DCM (100 mL*3). The organic phase is dried with anhydrous sodium sulfate, filtrated and evaporated to dryness under reduced pressure. The residue is purified by reverse-phase column chromatography (0-65% ACN in pure water) to give N3T12-027-01-S(612 mg, 0.851 mmol, 48.96%) as a white solid. LCMS: m/z 719.3 [(M+H)⁺]. ¹H NMR (400 MHz, DMSO-d₆) δ 11.34 (s, 1H), 7.65 (dd, J=11.0, 7.8 Hz, 1H), 7.41-7.35 (m, 2H), 7.32-7.19 (m, 7H), 6.88 (dt, J=8.8, 4.8 Hz, 4H), 5.59 (dd, J=9.2, 7.8 Hz, 1H), 5.08 (d, J=13.2 Hz, 2H), 4.06-3.94 (m, 1H), 3.81-3.72 (m, 7H), 3.71-3.60 (m, 3H), 3.59-3.43 (m, 2H), 3.12-2.91 (m, 2H), 2.75 (t, J=5.8 Hz, 1H), 2.63 (dd, J=9.2, 3.4 Hz, 1H), 1.17-1.06 (m, 9H), 0.98 (d, J=6.6 Hz, 3H). ³¹P NMR (400 MHz, DMSO-d₆) δ 148.59, 148.17.

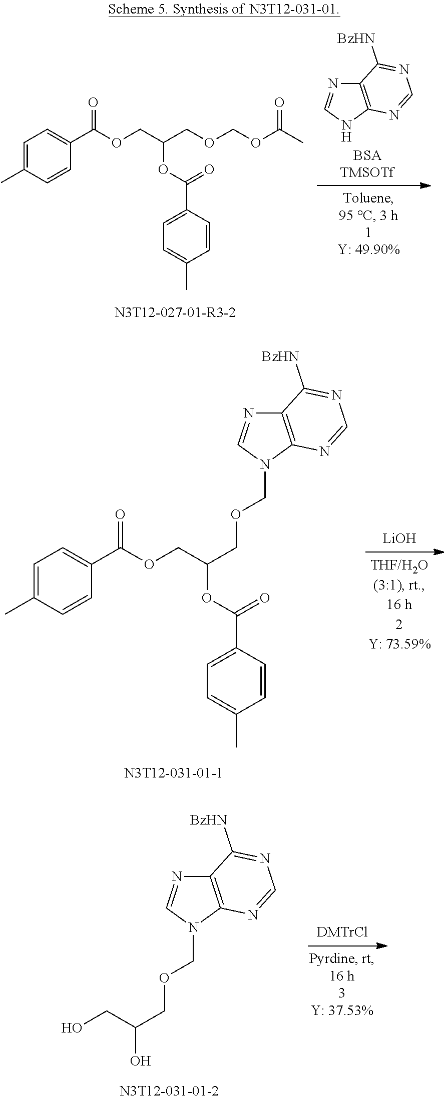

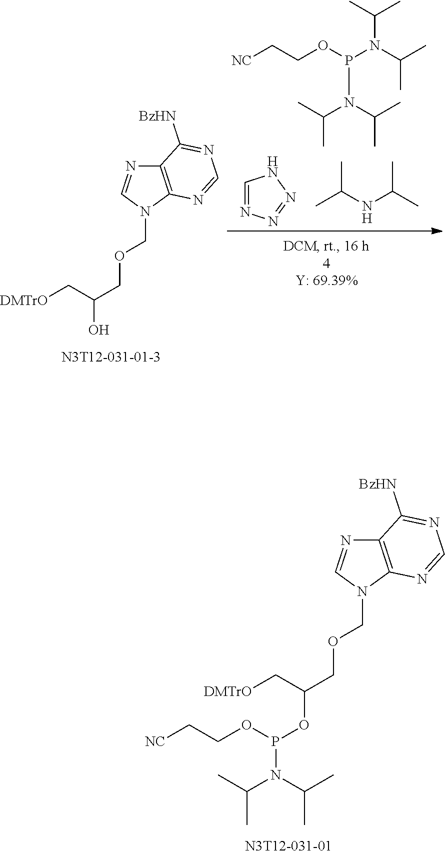

N3T12-031-01-1: To a solution of N-(9H-purin-6-yl)benzamide (3.2 g, 13.486 mmol) and N3T12-027-R3-2 (5.4 g, 13.486 mmol) in dry toluene is added (1E)-N-(trimethylsilyl)-1-[(trimethylsilyl)oxy]ethanimine (6.0 g, 29.668 mmol). The mixture is heated at 95° C. for 20 min. After cooling to room temperature (RT), trimethylsilyl trifluoromethanesulfonate (TMSOTf) (4.5 g, 20.228 mmol) is added to above reaction mixture at 0° C. The solution is stirred for 5 min before heating to 95° C. for 3 h. The solvent is evaporated to dryness under reduced pressure. The residue is purified by column chromatography over silica gel (DCM:MeOH=98:2) to give N3T12-031-01-1 (3.9 g, 6.729 mmol, 49.90%) as a colorless oil. LCMS: m/z 580.4 [(M+H)⁺].

N3T12-031-01-2: To a solution of N3T12-031-01-1 (3.9 g, 6.729 mmol) in THF (15 mL) and H₂O (5.00 mL) is added LiOH (0.7 g, 16.822 mmol). The mixture is stirred at RT overnight in the sealed tube. After the reaction is completed, HCl (4 M in dioxane) is added to adjust pH to 7. Then, the solvent is evaporated to dryness under reduced pressure. The residue is purified by column chromatography over silica gel (DCM:MeOH=95:5) to give N3T12-031-01-2 (1.7 g, 4.951 mmol, 73.59%) as a white solid. LCMS: m/z 344.4 [(M+H)⁺].

N3T12-031-01-3: To a solution of N3T12-031-01-2 (1.7 g, 4.951 mmol) in pyridine (30 mL) is added DMTrCl (1.8 g, 5.446 mmol) in 10 mL THF. The resulting mixture is stirred overnight at RT. Then, water (10 mL) is added to quench the reaction and evaporated to dryness under reduced pressure. The residue is purified by column chromatography over silica gel (DCM:MeOH=94:4) to give N3T12-031-01-3 (1.2 g, 1.858 mmol, 37.53%) as a white solid. LCMS: m/z 646.5 [(M+H)⁺].

N3T12-031-01: To a solution of N3T12-031-03 (1.1 g, 1.704 mmol) and di(prop-2-yl) ammonium 1,2,3,4-tetraazol-1-ide (0.44 g, 2.555 mmol) in DCM (8 mL) is added 3-{[2,6-dimethyl-3,5-di(prop-2-yl)-3,5-diaza-4-phosphahept-4-yl]oxy}propanenitrile (1.03 g, 3.407 mmol) in DCM (4 mL). The mixture is stirred at RT overnight in the sealed tube. After the reaction is completed, the mixture is poured into a saturated sodium bicarbonate solution (100 mL) and extracted with DCM (100 mL×3). The organic phase is dried with anhydrous sodium sulfate, filtrated and evaporated to dryness under reduced pressure. The residue is purified by reversed phase column (0-65% ACN in pure water) to N3T12-031-01 (1.0 g, 1.182 mmol, 69.39%) as a white solid. LCMS: m/z 846.6 [(M+H)⁺]. ¹H NMR (400 MHz, DMSO-d₆) δ 11.22 (d, J=8.2 Hz, 1H), 8.74 (d, J=11.2 Hz, 1H), 8.59 (d, J=12.4 Hz, 1H), 8.05 (d, J=7.6 Hz, 2H), 7.65 (t, J=7.2 Hz, 1H), 7.56 (t, J=7.6 Hz, 2H), 7.34-7.24 (m, 4H), 7.22-7.14 (m, 5H), 6.89-6.81 (m, 4H), 5.69 (d, J=15.2 Hz, 2H), 4.05-3.93 (m, 1H), 3.87-3.75 (m, 1H), 3.75-3.68 (m, 7H), 3.68-3.54 (m, 2H), 3.54-3.37 (m, 2H), 3.09-2.89 (m, 2H), 2.71 (dd, J=6.4, 5.4 Hz, 1H), 2.61 (dd, J=9.4, 3.6 Hz, 1H), 1.08 (dd, J=11.2, 6.8 Hz, 6H), 0.99 (d, J=6.6 Hz, 3H), 0.93 (d, J=6.8 Hz, 3H). ³¹P NMR (400 MHz, DMSO-d₆) δ 148.65 (s), 148.16 (s).

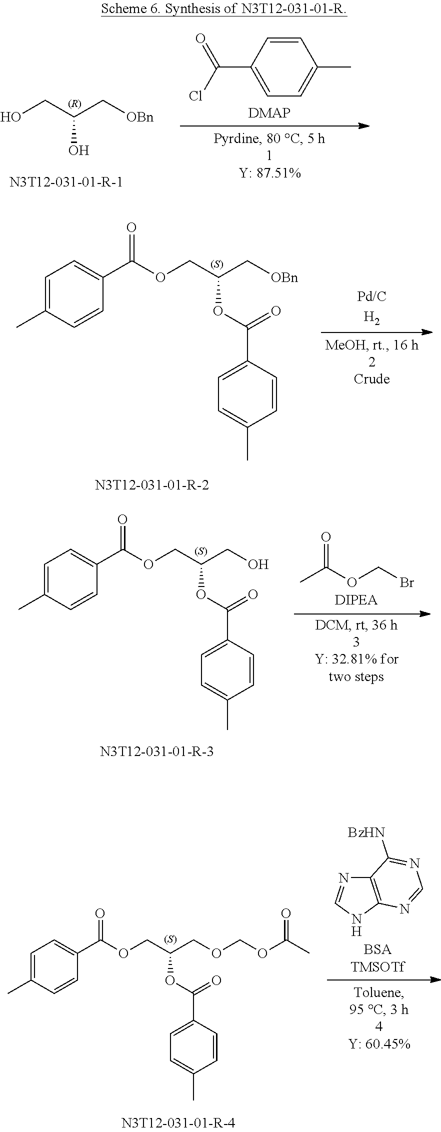

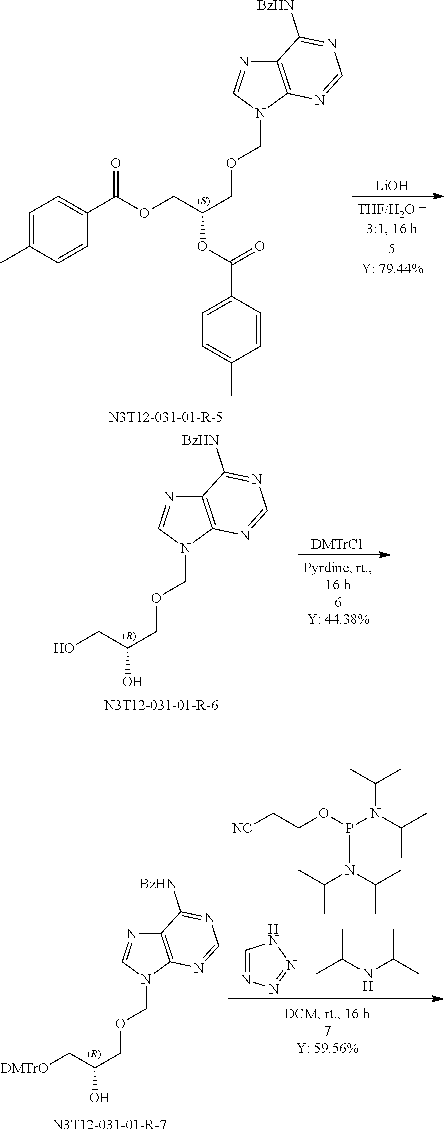

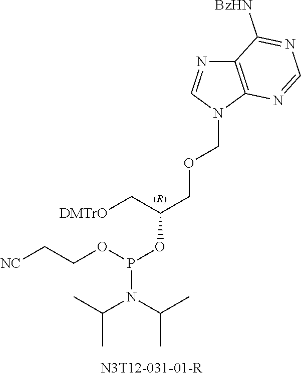

N3T12-031-01-R-2: To a solution of N3T12-031-01-R-1 (10.0 g, 54.879 mmol) in pyridine (250 mL) is added 4-methylbenzoyl chloride (25.4 g, 164.637 mmol) and DMAP (6.7 g, 54.879 mmol). The resulting mixture is stirred for 5 hours at 80° C. Then, water (50 mL) is added to quench the reaction and evaporated to dryness under reduced pressure. The residue is dissolved with DCM (300 mL) and washed with saturated sodium bicarbonate solution (150 mL*3). The organic phase is dried with anhydrous sodium sulfate, filtrated and evaporated to dryness under reduced pressure. The residue is purified by column chromatography over silica gel (PE/EA=15:1) to give N3T12-031-01-R-2 (20.1 g, 48.030 mmol, 87.51%) as a colorless oil. LCMS: m/z 441.3 [(M+Na)⁺].

N3T12-031-01-R-3: To a solution of N3T12-031-01-R-2 in MeOH (80 mL) is added Pd/C (1.2 g, Palladium on activated carbon (5%) (wetted with ca. 55% Water)). The mixture is stirred at room temperature for 16 hours under H₂ atmosphere. After the reaction is complete, solid is filtered and the solvent is evaporated to dryness under reduced pressure to afford the N3T12-031-01-R-3 (10 g, crude) as colorless oil. The crude is used for the next step without further purification. LCMS: m/z 351.3 [(M+Na)⁺].

N3T12-031-01-R-4: To a solution of N3T12-031-01-R-3 (10.0 g, 30.454 mmol) in DCM (100 mL) is added ethyl [di(prop-2-yl)]amine (11.8 g, 91.363 mmol) and bromomethyl acetate (9.3 g, 60.909 mmol). The mixture is stirred at room temperature for 36 hours. After the reaction is complete, the solvent is evaporated to dryness under reduced pressure. The residue is purified by column chromatography (PE/EA=90:10) over silica gel to give N3T12-031-01-R-4 (4.0 g, 9.989 mmol, 32.81% for two steps) as a colorless oil. LCMS: m/z 423.3 [(M+Na)⁺].

N3T12-031-01-R-5: To a suspension of N-(9H-purin-6-yl)benzamide (2.39 g, 9.989 mmol) and N3T12-031-01-R-4 (4.0 g, 9.989 mmol) in dry Toluene is added (1E)-N-(trimethylsilyl)-1-[(trimethylsilyl)oxy]ethanimine (4.47 g, 21.976 mmol). The mixture is heated to 95° C. for 20 minutes. After cooling to room temperature, TMSOTf (3.33 g, 14.984 mmol) is added to the above reaction mixture at 0° C. The solution is stirred for 5 minutes before heating to 95° C. for 2.5 hours. The solvent is evaporated to dryness under reduced pressure. The residue is purified by column chromatography over silica gel (DCM:MeOH=98:2) to give N3T12-031-01-R-5 (3.5 g, 6.039 mmol, 60.45%) as a colorless oil. LCMS: m/z 580.4 [(M+H)⁺].

N3T12-031-01-R-6: To a solution of N3T12-031-01-R-5 (3.4 g, 5.866 mmol) in THF (15 mL) and H₂O (5 mL) is added LiOH (0.62 g, 14.665 mmol). The mixture is stirred at room temperature for overnight in a sealed tube. After the reaction is complete, HCl (4 M in dioxane) is added to adjust pH to 7. Then, the solvent is evaporated to dryness under reduced pressure. The residue is purified by column chromatography over silica gel (DCM:MeOH=95:5) to give N3T12-031-01-R-6 (1.6 g, 4.660 mmol, 79.44%) as a white solid. LCMS: m/z 344.2 [(M+H)⁺].

N3T12-031-01-R-7: To a solution of N3T12-031-01-R-6 (1.5 g, 3.315 mmol) in Pyridine (10 mL) is added DMTrCl (1.24 g, 3.647 mmol) in 10 mL THF. The resulting mixture is stirred overnight at room temeprature. Then, water (10 mL) is added to quench the reaction and evaporated to dryness under reduced pressure. The residue is purified by column chromatography over silica gel (DCM:MeOH=94:4) to give N3T12-031-01-R-7 (950 mg, 1.471 mmol, 44.38%) as a white solid. LCMS: m/z 646.5 [(M+H)⁺]. Chiral HPLC: e.e. 99.36%.

N3T12-031-01-R: To a solution of N3T12-031-01-R-7 (910 mg, 1.409 mmol) and di(prop-2-yl) ammonium 1,2,3,4-tetraazol-1-ide (362.01 mg, 2.114 mmol) in DCM (10 mL) is added 3-{[2,6-dimethyl-3,5-di(prop-2-yl)-3,5-diaza-4-phosphahept-4-yl]oxy}propanenitrile (849.54 mg, 2.819 mmol) in DCM (2 mL). The mixture is stirred at room temperature overnight in a sealed tube. After the reaction is complete, the mixture is poured into a saturated sodium bicarbonate solution (100 mL). Then, the solution is extracted with DCM (100*3). The organic phase is dried with anhydrous sodium sulfate, filtrated and evaporated to dryness under reduced pressure. The residue is purified by reverse-phase column chromatography (0-65% ACN in pure water) to give N3T12-031-01-R (710 mg, 0.839 mmol, 59.56%) as a white solid. LCMS: m/z 846.3 [(M+H)⁺]. ¹H NMR (400 MHz, DMSO-d₆) δ 11.22 (d, J=8.2 Hz, 1H), 8.74 (d, J=11.2 Hz, 1H), 8.59 (d, J=12.4 Hz, 1H), 8.05 (d, J=7.6 Hz, 2H), 7.65 (t, J=7.2 Hz, 1H), 7.56 (t, J=7.6 Hz, 2H), 7.34-7.24 (m, 4H), 7.22-7.14 (m, 5H), 6.89-6.81 (m, 4H), 5.69 (d, J=15.2 Hz, 2H), 4.05-3.93 (m, 1H), 3.87-3.75 (m, 1H), 3.75-3.68 (m, 7H), 3.68-3.54 (m, 2H), 3.54-3.37 (m, 2H), 3.09-2.89 (m, 2H), 2.71 (dd, J=6.4, 5.4 Hz, 1H), 2.61 (dd, J=9.4, 3.6 Hz, 1H), 1.08 (dd, J=11.2, 6.8 Hz, 6H), 0.99 (d, J=6.6 Hz, 3H), 0.93 (d, J=6.8 Hz, 3H). ³¹P NMR (400 MHz, DMSO-d₆) δ 148.65 (s), 148.16 (s).

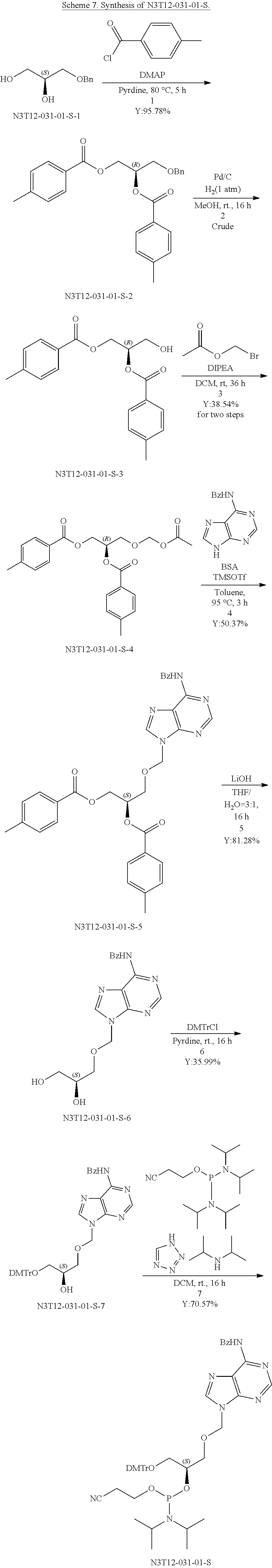

N3T12-031-01-S-2: To a solution of(S)-3-(benzyloxy) propane-1,2-diol (10.0 g, 54.879 mmol) in dry pyridine (250 mL) is added 4-methylbenzoyl chloride (25.4 g, 164.637 mmol) and DMAP (6.7 g, 54.879 mmol). The resulting mixture is stirred for 5 hours at 80° C. Then, saturated sodium bicarbonate solution (100 mL) is added to quench the reaction and the mixture is extracted with DCM (3*100 mL). The organic phase is dried with anhydrous sodium sulfate, filtrated and evaporated to dryness under reduced pressure. The residue is purified by column chromatography over silica gel (eluting with 0%-10% PE in EA) to give N3T12-031-01-S-2 (22.0 g, 52.570 mmol, 95.78%) as colorless oil. LCMS: m/z 441.2 [(M+Na)⁺].

N3T12-031-01-S-3: To a solution of N3T12-031-01-S-2 (12.0 g, 28.675 mmol) in MeOH (80 mL) is added Pd/C (1.2 g, Palladium on activated carbon (10%) (wetted with ca. 55% Water)). The mixture is stirred at room temperature for 16 hours under H₂ atmosphere (1 atm). After the reaction is complete, solid is filtered and the solvent is evaporated to dryness under reduced pressure to afford the N3T12-031-01-S-3 (10 g, crude) as a colorless oil. The crude is used for the next step without further purification. LCMS: m/z 351.2 [(M+Na)⁺].

N3T12-031-01-S-4: To a solution of N3T12-031-01-S-3 (10.0 g, 30.454 mmol) in DCM (100 mL) is added ethyl[di(prop-2-yl)]amine (11.8 g, 91.363 mmol) and bromomethyl acetate (9.3 g, 60.909 mmol). The mixture is stirred at room temperature for 36 hours. After the reaction is complete, the solvent is evaporated to dryness under reduced pressure. The residue is purified by silica column chromatography (eluting with 0%-10% EA in PE) to give N3T12-031-01-S-4 (4.7 g, 11.737 mmol, 38.54% for two steps) as a colorless oil. LCMS: m/z 423.1 [(M+Na)⁺].

N3T12-031-01-S-5: To a suspension of N-(9H-purin-6-yl)benzamide (2.2 g, 9.240 mmol) and N3T12-031-01-S-4 (3.7 g, 9.240 mmol) in dry toluene is added (1E)-N-(trimethylsilyl)-1-[(trimethylsilyl)oxy]ethanimine (4.1 g, 20.328 mmol). The mixture is heated to 95° C. for 20 minutes. After cooling to room temperature, TMSOTf (3.1 g, 13.860 mmol) is added to the reaction mixture at 0° C. The solution is stirred for 5 minutes before heating to 95° C. for 3 hours. The solvent is evaporated to dryness under reduced pressure. The residue is purified by column chromatography over silica gel (eluting with 0%-2% MeOH in DCM) to give N3T12-031-01-S-5 (2.7 g, 4.658 mmol, 50.37%) as a colorless oil. LCMS: m/z 580.3 [(M+H)⁺].

N3T12-031-01-S-6: To a solution of N3T12-031-01-S-5 (2.7 g, 4.658 mmol) in THF (15 mL) and H₂O (5 mL) is added LiOH (0.5 g, 11.646 mmol). The mixture is stirred at room temperature for 16 hours in a sealed tube. After the reaction is complete, HCl (4 M in dioxane) is added to adjust pH to 7. Then, the solvent is evaporated to dryness under reduced pressure. The residue is purified by column chromatography over silica gel (0-10% MeOH in DCM) to give N3T12-031-01-S-6 (1.3 g, 3.786 mmol, 81.28%) as a white solid. LCMS: m/z 344.2 [(M+H)⁺].

N3T12-031-01-S-7: To a solution of N3T12-031-01-S-6 (1.3 g, 3.786 mmol) in dry pyridine (10 mL) is added DMTrCl (1.4 g, 4.165 mmol) in 10 mL THF. The resulting mixture is stirred for 16 hours at room temperature under N₂ atmosphere. Then, water (30 mL) is added to quench the reaction and the solution is extracted with DCM (50 mL*3). The organic phase is dried with anhydrous sodium sulfate, filtrated and evaporated to dryness under reduced pressure. The residue is purified by column chromatography over silica gel (eluting with 0-3% MeOH in DCM) to give N3T12-031-01-S-7 (880 mg, 1.363 mmol, 35.99%) as a white solid. LCMS: m/z 646.5 [(M+H)⁺].

N3T12-031-01-S: To a solution of N3T12-031-01-S-7 (570 mg, 0.883 mmol) and di(prop-2-yl) ammonium 1,2,3,4-tetraazol-1-ide (226 mg, 1.324 mmol) in DCM (10 mL) is added 3-{[2,6-dimethyl-3,5-di(prop-2-yl)-3,5-diaza-4-phosphahept-4-yl]oxy}propanenitrile (532 mg, 1.765 mmol) in DCM (2 mL). The mixture is stirred at room temperature for 16 hours in a sealed tube under N₂ atmosphere. After the reaction is complete, the mixture is poured into a saturated sodium bicarbonate solution (50 mL). Then, the solution is extracted with DCM (50 mL*3). The organic phase is dried with anhydrous sodium sulfate, filtrated and evaporated to dryness under reduced pressure. The residue is purified by reverse-phase column chromatography (0-65% ACN in pure water) to give N3T12-031-01-S(527 mg, 0.623 mmol, 70.57%) as a white solid. LCMS: m/z 846.4 [(M+H)⁺]. ¹H NMR (400 MHz, d6-DMSO) δ 11.22 (d, J=8.0 Hz, 1H), 8.74 (d, J=11.2 Hz, 1H), 8.59 (d, J=12.4 Hz, 1H), 8.05 (d, J=7.6 Hz, 2H), 7.65 (t, J=7.2 Hz, 1H), 7.56 (t, J=7.6 Hz, 2H), 7.34-7.24 (m, 4H), 7.22-7.14 (m, 5H), 6.89-6.81 (m, 4H), 5.69 (d, J=15.2 Hz, 2H), 4.05-3.93 (m, 1H), 3.87-3.75 (m, 1H), 3.75-3.68 (m, 7H), 3.68-3.54 (m, 2H), 3.54-3.37 (m, 2H), 3.09-2.89 (m, 2H), 2.71 (dd, J=6.4, 5.4 Hz, 1H), 2.61 (dd, J=9.4, 3.6 Hz, 1H), 1.08 (dd, J=11.2, 6.8 Hz, 6H), 0.99 (d, J=6.6 Hz, 3H), 0.93 (d, J=6.8 Hz, 3H). ³¹P NMR (400 MHz, DMSO-d₆) δ 148.65, 148.16.

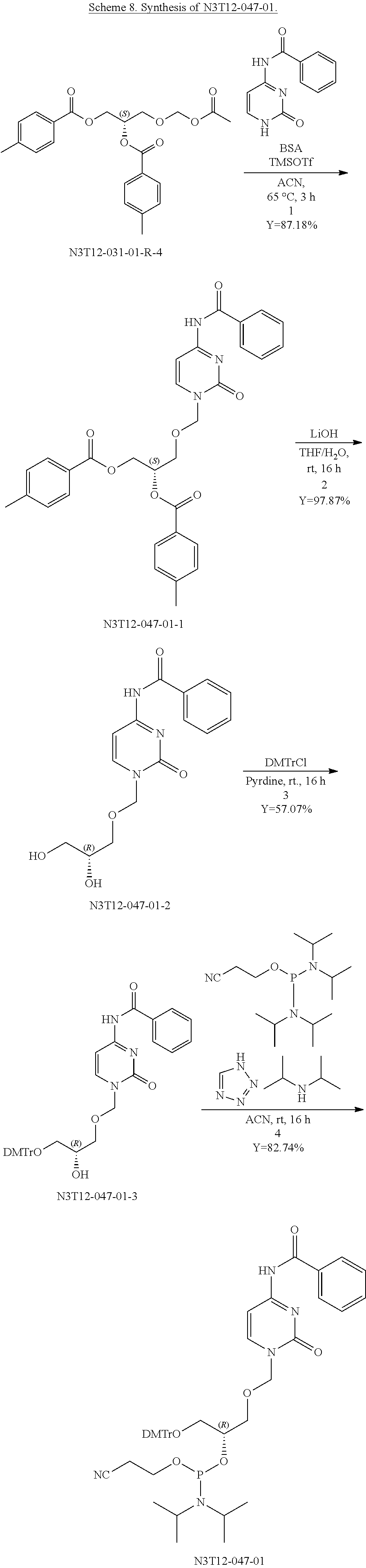

N3T12-047-01-1: To a solution of N-(2-oxo-1H-pyrimidin-4-yl)benzamide (1.00 g, 4.645 mmol) and N3T12-031-01-R-4 (1.24 g, 3.097 mmol) in ACN (15 mL) is added BSA (1.39 g, 6.813 mmol) at room temperature under N₂ atmosphere. The reaction is stirred for 30 minutes at 65° C. Then, TMSOTf (1.03 g, 4.645 mmol) is added. The reaction is stirred for 3 hours at 65° C. under N₂ atmosphere. The reaction is quenched by NaHCO₃(aq, 20 mL) and the mixture is extracted with DCM (3*30 mL). The combined organic layer is dried over Na₂SO₄ and the solvent is removed in vacuo. The residue is purified by flash column chromatography (eluting with 0%-7% MeOH in DCM) to afford N3T12-047-01-1 (1.5 g, 2.700 mmol, 87.18%) as a white solid. LCMS: m/z 556.3 [(M+H)⁺].

N3T12-047-01-2: To a solution of N3T12-047-01-1 (1.6 g, 2.880 mmol) in tetrahydrofuran (21.6 mL) is added lithium hydroxide (7.2 mL, 7.200 mmol, 1 M in H₂O). The reaction is stirred for 16 hours at room temperature. The mixture is adjusted to pH=7 by HCl (4M, in dioxane) and the solvent is removed in vacuo. The residue is purified by flash column chromatography (eluting with 0%-15% MeOH in DCM) to afford N3T12-047-01-2 (0.9 g, 2.818 mmol, 97.87%) as a white solid. LCMS: m/z 320.3 [(M+H)⁺]. ¹H NMR (400 MHz, DMSO-d₆) δ 11.26 (s, 1H), 8.20 (d, J=6.6 Hz, 1H), 8.02-7.97 (m, 2H), 7.62 (t, J=7.4 Hz, 1H), 7.53-7.49 (m, 2H), 7.30 (s, 1H), 5.25 (s, 2H), 4.86 (d, J=4.8 Hz, 1H), 4.64 (t, J=5.6 Hz, 1H), 3.58-3.54 (m, 2H), 3.45-3.42 (m, 1H), 3.30 (t, J=5.4 Hz, 2H).

N3T12-047-01-3: To a solution of N3T12-047-01-2 (0.9 g, 2.818 mmol) in Pyrdine (15 mL) is added 1-[chloro(4-methoxyphenyl)phenylmethyl]-4-methoxybenzene (1.24 g, 3.664 mmol) slowly at 0° C. The reaction is stirred for 16 hours at room temperature under N₂ atmosphere. The reaction is quenched by H₂O (30 mL) and the mixture is extracted with DCM (3*30 mL). The combined organic layer is dried over Na₂SO₄ and the solvent is removed in vacuo. The residue is purified by flash column chromatography (eluting with 0%-7% MeOH in DCM) to afford N3T12-047-01-3 (1 g, 1.609 mmol, 57.07%) as a light-yellow solid. LCMS: m/z 622.2 [(M+H)⁺].

N3T12-047-01: To a solution of N3T12-047-01-3 (650 mg, 1.046 mmol) in dry ACN (10 mL) is added Diisopropylammonium Tetrazolide (269 mg, 1.568 mmol) and 3-{[2,6-dimethyl-3,5-di(prop-2-yl)-3,5-diaza-4-phosphahept-4-yl]oxy}propanenitrile (630 mg, 2.091 mmol) at 0° C. under N₂ atmosphere. The reaction is stirred for 16 hours at room temperature. The reaction is extracted with NaHCO₃(aq)/DCM (3*20 mL) and the combined organic layer is dried over Na₂SO₄. The solvent is removed in vacuo and the residue is purified by reverse-phase column chromatography (eluting with 0%-60% ACN in H2O) to afford N3T12-047-01 (711 mg, 0.865 mmol, 82.74%) as a white solid. LCMS: m/z 761.2 [(M-DIPA+Na)⁺]. ¹H NMR (400 MHz, DMSO-d₆) δ 11.29 (s, 1H), 8.11 (dd, J=10.6, 7.8 Hz, 1H), 8.01 (d, J=7.8 Hz, 2H), 7.63 (t, J=7.4 Hz, 1H), 7.52 (t, J=7.6 Hz, 2H), 7.40-7.35 (m, 2H), 7.35-7.16 (m, 8H), 6.91-6.82 (m, 4H), 5.24 (d, J=12.2 Hz, 2H), 4.12-3.96 (m, 1H), 3.85-3.62 (m, 10H), 3.58-3.43 (m, 2H), 3.10-2.92 (m, 2H), 2.76 (t, J=5.8 Hz, 1H), 2.64 (t, J=6.2 Hz, 1H), 1.13-1.09 (m, 9H), 0.98 (d, J=6.7 Hz, 3H). ³¹P NMR (162 MHz, DMSO-d₆) δ 148.69, 148.07.

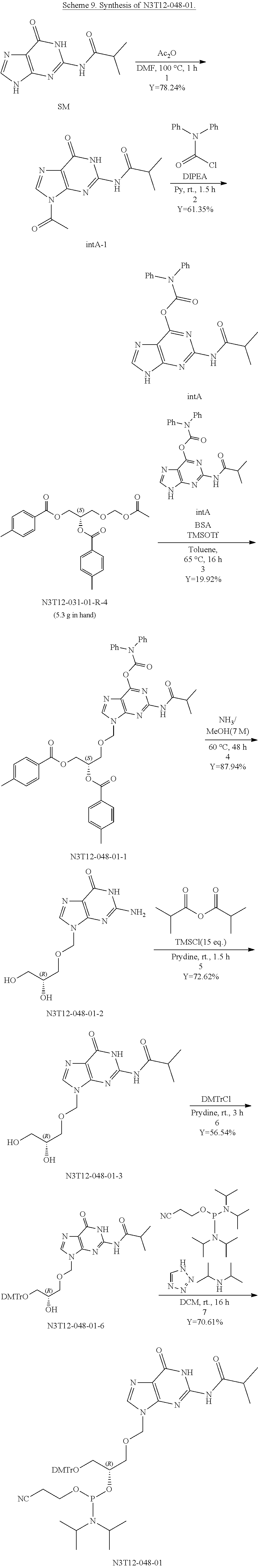

intA-1: Dried 2-methyl-N-(6-oxo-1,9-dihydropurin-2-yl) propanamide (29.0 g, 131.091 mmol) is suspended in dry DMF (300 mL) before Ac₂O (33.5 g, 327.728 mmol) is added and the mixture heated to 100° C. for 1 hour. After the reaction is complete, the clear solution is evaporated to dryness and the solid residue is suspended in dioxane and stirred overnight. Crystals are filtered and dried to afford intA-1 (27.1 g, 102.560 mmol, 78.24%) as a white solid. The crude is used for the next step without further purification. LCMS: m/z 264.1 [(M+H)⁺].

intA: The acetyl derivative N-(9-acetyl-6-oxo-6,9-dihydro-1H-purin-2-yl) isobutyramide (27.1 g, 102.940 mmol) is co-evaporated with pyridine (80 mL*3) and suspended in dry pyridine (300 mL), to which DIPEA (26.6 g, 205.880 mmol) and diphenylcarbamoyl chloride (31.0 g, 133.822 mmol) are added and the mixture stirred at room temperature for 1.5 hours under N₂ atmosphere. After the reaction is complete, water (20 mL) is added to quench the reaction, and after 10 minutes the solution is evaporated. The residue is boiled with ethanol-water (500 mL, 1:1) for 1 hour. After cooling to room temperature, crystals are filtered and dried to afford product intA (26.3 g, 63.154 mmol, 61.35%) as a white solid. LCMS: m/z 417.1 [(M+H)⁺].

N3T12-048-01-1: A suspension of intA (13.85 g, 33.264 mmol) and BSA (16.92 g, 83.161 mmol) in dry Tolune (200 mL) is stirred at 65° C. for 30 minutes under N₂ atmosphere. Then, N3T12-031-01-R-4 (11.10 g, 27.720 mmol) and TMSOTf (13.18 g, 58.212 mmol) are added, respectively, at 65° C. The mixture is stirred at 65° C. for another 16 hours. After the reaction is complete, the solvent is poured into 250 mL DCM and washed with saturated sodium bicarbonate (150 mL*3). The organic phase is dried with anhydrous sodium sulfate, filtrated and evaporated to dryness under reduced pressure. The residue is purified by column chromatography over silica gel (0%-30% EA in DCM) to afford N3T12-048-01-1 (4.18 g, 5.523 mmol, 19.92%) as light yellow solid. LCMS: m/z 757.1 [(M+H)⁺].

N3T12-048-01-2: N3T12-048-01-1 (4.08 g, 5.391 mmol) in NH₃/MeOH (40 mL, 7 M) is stirred at 60° C. for 48 h. After the reaction is complete, the solvent is evaporated to dryness under reduced pressure. The residue is purified by column chromatography over silica gel (0%-40% MeOH in DCM) to give N3T12-048-01-2 (1.21 g, 4.741 mmol, 87.94%) as a white solid. ¹H NMR (400 MHz, DMSO-d₆) δ 10.64 (s, 1H), 7.81 (s, 1H), 6.52 (s, 2H), 5.34 (s, 2H), 4.75 (s, 1H), 4.52 (s, 1H), 3.55-3.20 (m, 5H).

N3T12-048-01-3: To a solution of N3T12-048-01-2 (1.21 g, 4.741 mmol) in dry pyridine (10 mL) is added TMSCl (7.73 g, 71.112 mmol) dropwise. The mixture is stirred at room temperature for 30 minutes. Then, Isobutyric anhydride (3.75 g, 23.704 mmol) is added at room temperature. The mixture is stirred at room temperature for another 1.5 hours. After the reaction is complete, 6 mL 25% ammonia aqueous solution is added and the mixture is stirred at room temperature for 30 minutes. Then, the mixture is concentrated under reduced pressure. The crude is purified by reverse-phase column chromatography (0%-20% ACN in 0.1% FA water) to afford N3T12-048-01-3 (1.12 g, 3.443 mmol, 72.62%) as a white solid. LCMS: m/z 326.2 [(M+H)⁺].

N3T12-048-01-6: To a solution of N3T12-048-01-3 (1.10 g, 3.381 mmol) in dry pyridine (10 mL) is added DMTrCl (1.60 g, 4.734 mmol) in 2 mL dry THF under N₂ atmosphere. The mixture is stirred at room temperature for 3 hours. Water (2 mL) is added to quench the reaction and the mixture is poured into water (50 mL). Then, the solution is extracted with DCM (50 mL*3). The organic phase is dried with anhydrous sodium sulfate, filtrated and evaporated to dryness under reduced pressure. The residue is purified by column chromatography over silica gel (0%-6% MeOH in DCM) to give N3T12-048-01-6 (1.20 g, 1.912 mmol, 56.54%) as a light yellow solid. LCMS: m/z 628.1 [(M+H)⁺].

N3T12-048-01: To a solution of N3T12-048-01-6 (700 mg, 1.115 mmol) and di(prop-2-yl) ammonium 1,2,3,4-tetraazol-1-ide (286 mg, 1.673 mmol) in dry DCM (10 mL) is added 3-{[2,6-dimethyl-3,5-di(prop-2-yl)-3,5-diaza-4-phosphahept-4-yl]oxy}propanenitrile (672 mg, 2.230 mmol) in dry DCM (1 mL) under N₂ atmosphere. The mixture is stirred at room temperature for 16 hours in a sealed tube. After the reaction is complete, the mixture is poured into a saturated sodium bicarbonate solution (50 mL). Then, the solution is extracted with DCM (50 mL*3). The organic phase is dried with anhydrous sodium sulfate, filtrated and evaporated to dryness under reduced pressure. The residue is purified by reversed phase column chromatography (0%-60% ACN in pure water) to give N3T12-048 (652 mg, 0.787 mmol, 70.61%) as a white solid. LCMS: m/z 828.3 [(M+H)⁺]. ¹H NMR (400 MHz, DMSO-d₆) δ 12.08 (s, 1H), 11.76 (s, 1H), 8.12 (d, J=12.6 Hz, 1H), 7.33-7.23 (m, 4H), 7.22-7.12 (m, 5H), 6.84 (t, J=8.6 Hz, 4H), 5.50-5.41 (m, 2H), 3.97-3.89 (m, 1H), 3.73 (d, J=3.4 Hz, 6H), 3.72-3.57 (m, 4H), 3.53-3.36 (m, 2H), 3.06-2.86 (m, 2H), 2.76 (dd, J=13.8, 6.8 Hz, 1H), 2.71-2.58 (m, 2H), 1.13-1.04 (m, 12H), 0.96 (dd, J=14.8, 6.7 Hz, 6H). ³¹P NMR (162 MHz, DMSO-d₆) δ 148.53, 148.16.

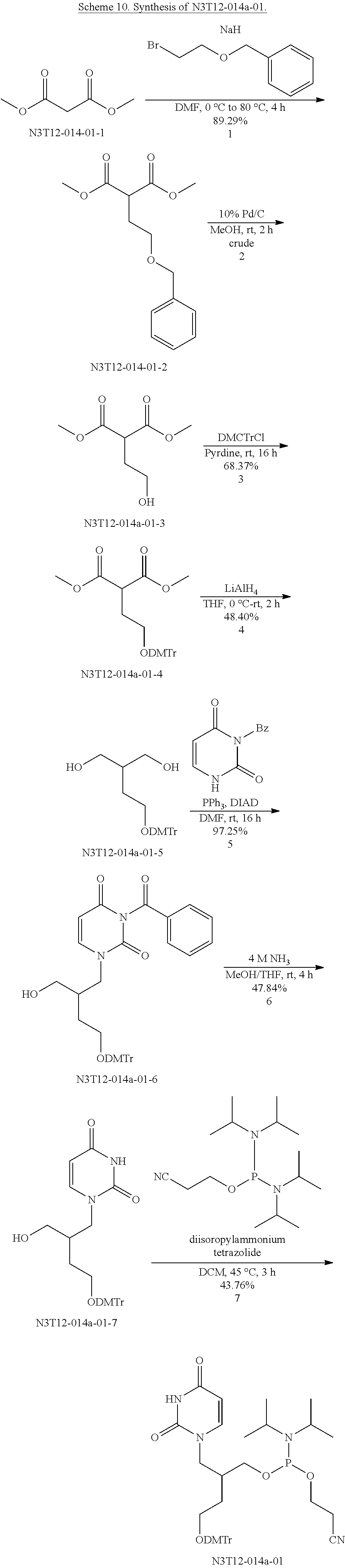

N3T12-014a-01-2: To a solution of sodium hydride (3.33 g, 83.258 mmol, 60% purity) in DMF (100 mL) is added methyl 3-methoxy-3-oxopropanoate (10 g, 75.689 mmol) at 0° C. under a nitrogen atmosphere. The mixture is stirred at 0° C. for 2 hours. Then, {[(2-bromoethyl)oxy]methyl}benzene (17.91 g, 83.258 mmol) is added and stirred at 80° C. for 4 hours. After the reaction is complete, the mixture is poured into a saturated ammonium chloride solution (150 mL) to quench the reaction and extracted with DCM (200 mL*3), dried with anhydrous sodium sulfate, filtrated, and evaporated to dryness under reduced pressure. The residue is purified by column chromatography (PE:EA=9:1) to give N3T12-014a-01-2 (18 g, 56.330 mmol, 89.29%) as a colorless oil. LCMS: m/z 267.4 [(M+H)⁺].

N3T12-014a-01-3: To a solution of N3T12-014a-01-2 (16 g, 60.085 mmol) in MeOH (150 mL) is added Pd/C (1.6 g, 10% purity). The mixture is stirred at room temperature for 16 hours under H₂ atmosphere. After the reaction is complete, solid is filtered and the solvent is evaporated to dryness under reduced pressure (35 Celsius) to afford the crude product/intermediate (16 g). The crude is used for the next step without further purification. LCMS: m/z 145.2 [(M−OCH₃)⁺].

N3T12-014a-01-4: To a solution of N3T12-014a-01-3 (10.5 g, 59.60 mmol) in Pyrdine (100 mL) is added 1-[chloro(4-methoxyphenyl)phenylmethyl]-4-methoxybenzene (30.29 g, 89.40 mmol) at 0° C. The reaction is stirred for 16 hours at room temperture under N₂ atmosphere. The reaction is quenched by H₂O (50 mL) and the mixture is extracted with DCM (3*60 mL). The combined organic layer is dried over Na₂SO₄ and the solvent is removed in vacuo (co-evaporated with toluene for three times). The residue is purified by flash column chromatography (eluting with 0%-15% EA in PE) to afford N3T12-014a-01-4 (19.5 g, 40.749 mmol, 68.37%) as a light-yellow oil. LCMS: m/z 501.5 [(M+H)⁺].

N3T12-014a-01-5: To a solution of N3T12-014a-01-4 (19.5 g, 36.674 mmol) in tetrahydrofuran (200 mL) is added LiAlH4 in tetrahydrofuran (2.5M,44 mL) at 0° C. The reaction is stirred for 2 hours at 0° C. Sodium sulfate decahydrate is slowly added to the reaction solution until no bubbles are produced. Then, the reaction mixture is filtrated and concentrated under reduced pressure to give a residue which is purified by silica gel column chromatography (SiO₂, DCM:MeOH=20:1) to give N3T12-014a-01-5 (7.5 g, 17.751 mmol, 48.40% yield) as a colourless oil. LCMS: m/z 445.5 [(M+Na)⁺]. ¹H NMR (400 MHz, DMSO) δ 7.39-7.21 (m, 9H), 6.91-6.86 (m, 4H), 4.28 (t, J=5.2 Hz, 2H), 3.73 (s, 6H), 3.30 (t, J=5.3 Hz, 4H), 2.98 (t, J=6.5 Hz, 2H), 1.64-1.50 (m, 3H).

N3T12-014a-01-6: To a solution of N3T12-014a-01-5 (1.55 g, 7.157 mmol) and triphenylphosphane (2.71 g, 10.338 mmol) in tetrahydrofuran (80 mL) is slowly added DIAD (2.09 g, 10.338 mmol) at 0° C. The reaction is stirred for 16 hours at room temperature. Water (60 mL) is added to the reaction solution, which is then stirred for 10 minutes before being extracted with EA (80 mL), dried over Na₂SO₄ and concentrated to give N3T12-014a-01-6 (4.8 g, 7.733 mmol, 97.25% yield) crude as a colourless oil. The crude product is used for the next step without further purification. LCMS: m/z 643.5 [(M+Na)⁺].

N3T12-014a-01-7: To a solution of N3T12-014a-01-6 (4.8 g, 7.733 mmol) in tetrahydrofuran (10 mL) is added NH₃/MeOH (40 mL). The reaction is stirred for 4 hours at room temperature. The reaction mixture is concentrated under reduced pressure to give a residue which is purified by silica gel column chromatography (SiO₂,DCM:MeOH=96:4) to give N3T12-014a-01-7 (1.8 g, 3.699 mmol, 47.84% yield) as a white solid. LCMS: m/z 539.5 [(M+Na)⁺].

N3T12-014a-01: To a solution of Diisopropylammonium Tetrazolide (0.89 g, 5.227 mmol) and N3T12-014a-01-7 (1.8 g, 3.484 mmol) in dry DCM (30 mL) is added 3-{[2,6-dimethyl-3,5-di(prop-2-yl)-3,5-diaza-4-phosphahept-4-yl]oxy}propanenitrile (2.10 g, 6.969 mmol) with nitrogen protection at 0° C. The reaction is stirred overnight at room temperature. A saturated NaHCO₃ aqueous solution (20 mL) is then added to the reaction mixture before being extracted with DCM (30 mL), dried over Na₂SO₄ and concentrated under reduced pressure to give a residue which is purified by reverse-phase column chromatography (ACN:H₂O=60-70%) to give N3T12-014a-01 (1.093 g, 1.525 mmol, 43.76% yield) as a white solid. LCMS: m/z 656.4 [(M hydrolyzed+Na)⁺]. ¹H NMR (400 MHz, DMSO-d₆) δ 11.25 (s, 1H), 7.58-7.46 (m, 1H), 7.38-7.33 (m, 2H), 7.29 (t, J=7.6 Hz, 2H), 7.24-7.19 (m, 5H), 6.87 (d, J=8.8 Hz, 4H), 5.54 (t, J=7.6 Hz, 1H), 3.73 (s, 6H), 3.72-3.35 (m, 8H), 3.10-2.93 (m, 2H), 2.77-2.66 (m, 2H), 2.19 (s, 1H), 1.67-1.45 (m, 2H), 1.25-0.92 (m, 12H).

The foregoing nucleoside analogs, prepared above in protected form, can be introduced into the antisense strand of a double-stranded siRNA duplex to reduce off-target effects.

### Example 3. In Vitro Evaluation of Human LRRK2 siRNAs by Transfection

The siRNAs are transfected into the Human Lung Epithelial cells A549 with RNAiMAX (Invitrogen, 13778075) in a 48-well plate. All the siRNAs are tested at 10 nM. 17 siRNAs are further tested at 1 nM, 0.1 nM, 0.01 nM.

48 hours after transfection, the culture medium is discarded and cells are collected for RNA extraction. Total RNA is extracted by FastPure Cell/Tissue Total RNA Isolation Kit V2 (Vazyme-RC112-01) according to the kit instructions and reversed transcribed to cDNA by HiScript III All-in-one RT SuperMix Perfect for qPCR (Vazyme-R333-01).

The cDNA of the target gene is quantified by qPCR with specific primer and probe sets. GAPDH cDNA is measured as a housekeeping gene in parallel. The expression of the target gene in each test sample is determined by relative quantitation (RQ) using the comparative Ct (ΔΔCt) method.

TABLE 2
In Vitro Screen with Human LRRK2
siRNAs (10 nM) by Transfection

| | Average % LRRK2 | |
|---|---|---|
| Duplex | mRNA Remaining | STDEV |
| N3T130001 | 23.1 | 1.75 |
| N3T130002 | 23.5 | 2.00 |
| N3T130003 | 26.6 | 0.02 |
| N3T130004 | 25.8 | 0.35 |
| N3T130005 | 131.8 | 16.60 |
| N3T130006 | 62.5 | 10.46 |
| N3T130007 | 50.3 | 2.01 |
| N3T130008 | 25.6 | 0.27 |
| N3T130009 | 43.1 | 4.38 |
| N3T130010 | 26.4 | 0.50 |
| N3T130011 | 50.3 | 5.02 |
| N3T130012 | 41.1 | 3.46 |
| N3T130013 | 27.8 | 1.66 |
| N3T130014 | 28.7 | 0.18 |
| N3T130015 | 36.4 | 4.08 |
| N3T130016 | 32.0 | 11.06 |
| N3T130017 | 28.4 | 0.47 |
| N3T130018 | 99.2 | 5.32 |
| N3T130019 | 35.5 | 2.87 |
| N3T130020 | 28.7 | 3.03 |
| N3T130021 | 28.9 | 4.35 |
| N3T130022 | 26.2 | 1.29 |
| N3T130023 | 24.8 | 1.57 |
| N3T130024 | 33.4 | 0.50 |
| N3T130025 | 29.6 | 0.52 |
| N3T130026 | 23.6 | 2.10 |
| N3T130027 | 38.1 | 2.52 |
| N3T130028 | 26.0 | 1.93 |
| N3T130029 | 28.1 | 0.26 |
| N3T130030 | 108.5 | 6.25 |
| N3T130031 | 30.6 | 1.29 |
| N3T130032 | 29.9 | 1.23 |
| N3T130033 | 32.7 | 6.50 |
| N3T130034 | 22.2 | 6.27 |
| N3T130035 | 20.4 | 0.87 |
| N3T130036 | 98.8 | 1.91 |
| N3T130037 | 41.1 | 4.70 |
| N3T130038 | 59.5 | 2.20 |
| N3T130039 | 37.5 | 3.60 |
| N3T130040 | 39.3 | 6.81 |
| N3T130041 | 29.6 | 1.30 |
| N3T130042 | 42.6 | 5.11 |
| N3T130043 | 55.2 | 5.67 |
| N3T130044 | 35.8 | 5.28 |
| N3T130045 | 34.7 | 0.43 |
| N3T130046 | 72.5 | 22.10 |
| N3T130047 | 32.4 | 4.83 |
| N3T130048 | 33.9 | 0.40 |
| N3T130049 | 35.5 | 4.92 |
| N3T130050 | 26.3 | 0.71 |
| N3T130051 | 29.3 | 0.27 |
| N3T130052 | 29.7 | 2.59 |
| N3T130053 | 30.9 | 1.59 |
| N3T130054 | 29.2 | 0.59 |
| N3T130055 | 24.5 | 0.03 |
| N3T130056 | 33.9 | 2.50 |
| N3T130057 | 24.1 | 0.70 |
| N3T130058 | 28.9 | 5.31 |
| N3T130059 | 14.6 | 0.76 |
| N3T130060 | 30.8 | 3.29 |
| N3T130061 | 39.7 | 1.17 |
| N3T130062 | 26.4 | 3.50 |
| N3T130063 | 31.2 | 3.71 |
| N3T130064 | 32.8 | 8.51 |
| N3T130065 | 40.9 | 7.50 |
| N3T130066 | 43.4 | 1.79 |
| N3T130067 | 75.8 | 0.27 |
| N3T130068 | 66.1 | 2.24 |
| N3T130069 | 24.1 | 3.92 |
| N3T130070 | 40.8 | 2.70 |
| N3T130071 | 55.3 | 4.43 |
| N3T130072 | 53.2 | 9.33 |
| N3T130073 | 67.3 | 6.66 |
| N3T130074 | 29.5 | 1.60 |
| N3T130075 | 53.1 | 2.36 |
| N3T130076 | 27.7 | 0.72 |
| N3T130077 | 29.8 | 2.43 |
| N3T130078 | 55.3 | 2.43 |
| N3T130079 | 32.0 | 0.34 |
| N3T130080 | 32.4 | 1.20 |
| N3T130081 | 71.4 | 4.60 |
| N3T130082 | 42.6 | 1.03 |
| N3T130083 | 90.6 | 17.63 |
| N3T130084 | 37.8 | 4.56 |
| N3T130085 | 110.9 | 4.82 |
| N3T130086 | 28.9 | 0.07 |
| N3T130087 | 26.3 | 5.24 |
| N3T130088 | 33.7 | 6.91 |
| N3T130089 | 38.2 | 1.95 |
| N3T130090 | 41.5 | 5.70 |
| N3T130091 | 20.6 | 0.78 |
| N3T130092 | 34.4 | 1.32 |
| N3T130093 | 48.1 | 1.99 |
| N3T130094 | 22.5 | 3.08 |
| N3T130095 | 21.1 | 2.35 |
| N3T130096 | 10.4 | 0.55 |
| N3T130097 | 17.6 | 1.79 |
| N3T130098 | 50.9 | 1.72 |
| N3T130099 | 13.3 | 0.37 |
| N3T130100 | 25.2 | 0.13 |

The percentage of human LRRK2 mRNA remaining in cultured A549 cells relative to mock transfection when normalized to GAPDH mRNA levels is determined for each compound following transfection into cells. 43 siRNA duplexes have more than 70% mRNA knockdown activity at 10 nM (Table 2). The fact that siRNA directed to different sites on the mRNA transcript provides considerable variation in efficacy indicates that there are “hotspots,” where the mRNA is amenable to suppression by siRNA. siRNAs which target these hotspots are likely to be more effective.

The top 20 siRNAs (10 nM) selected for activity in the above assay are listed in Table 2a:

TABLE 2a

| | SEQ | | | SEQ | | |
|---|---|---|---|---|---|---|
| Duplex | ID NO | sense strand (5′ to 3′) | Range* | ID NO | antisense strand (5′ to 3′) | Range* |
| N3T130100-01 | 199 | GACGAGGAAACUCUGAAGA | 169 | 200 | UCUUCAGAGUUUCCUCGUC | 167 |
| | | AG | | | CU | |
| N3T130001-01 | 1 | GUUGGUCACUUCUGUGCAA | 377 | 2 | UUGCACAGAAGUGACCAAC | 375 |
| | | AU | | | CC | |
| N3T130008-01 | 15 | GAGUGGCAAUGUCAGGUGU | 864 | 16 | ACACCUGACAUUGCCACUC | 862 |
| | | UA | | | AU | |
| N3T130023-01 | 45 | GGCAAUGUCAGGUGUUAUA | 868 | 46 | UAUAACACCUGACAUUGCC | 866 |
| | | AU | | | AC | |
| N3T130002-01 | 3 | GGGAUUCAGAAAUGUGGAU | 1807 | 4 | AUCCACAUUUCUGAAUCCC | 1805 |
| | | UA | | | AG | |
| N3T130094-01 | 187 | GAGCGUGUGAUCAGAAUAA | 2279 | 188 | UUAUUCUGAUCACACGCUC | 2277 |
| | | CA | | | UC | |
| N3T130095-01 | 189 | GAUCUCGUGAACAAGAUGU | 2429 | 190 | ACAUCUUGUUCACGAGAUC | 2427 |
| | | AC | | | CA | |
| N3T130096-01 | 191 | GGACUUGCACAGUAAUAAA | 3255 | 192 | UUUAUUACUGUGCAAGUCC | 3253 |
| | | UU | | | AA | |
| N3T130004-01 | 7 | GACCCUCAGUGGUUUUAGA | 3347 | 8 | UCUAAAACCACUGAGGGUC | 3345 |
| | | UC | | | CA | |
| N3T130099-01 | 197 | GGUCUUUAGAUAUGAGCAG | 3731 | 198 | CUGCUCAUAUCUAAAGACC | 3729 |
| | | CA | | | GC | |
| N3T130035-01 | 69 | GAUAUGAGCAGCAAUGAUA | 3739 | 70 | UAUCAUUGCUGCUCAUAUC | 3737 |
| | | UU | | | UA | |
| N3T130069-01 | 137 | GGGAACUCUUAUUUAGCCA | 3803 | 138 | UGGCUAAAUAAGAGUUCCC | 3801 |
| | | UA | | | UU | |
| N3T130028-01 | 55 | GGAACUCUUAUUUAGCCAU | 3804 | 56 | AUGGCUAAAUAAGAGUUCC | 3802 |
| | | AA | | | CU | |
| N3T130034-01 | 67 | GCCAUAAUCAGAUCAGCAU | 3818 | 68 | AUGCUGAUCUGAUUAUGGC | 3816 |
| | | CU | | | UA | |
| N3T130097-01 | 193 | GGUCGUGAGGAAUUCUAUA | 4324 | 194 | UAUAGAAUUCCUCACGACC | 4322 |
| | | GU | | | UG | |
| N3T130059-01 | 117 | GUGAAUGCCACCGAGGAAU | 4597 | 118 | AUUCCUCGGUGGCAUUCAC | 4595 |
| | | CU | | | AA | |
| N3T130026-01 | 51 | GCCUUAUUUUCCAAUGGGA | 5226 | 52 | UCCCAUUGGAAAAUAAGGC | 5224 |
| | | UU | | | AU | |
| N3T130055-01 | 109 | GGAUCUGAAGUCUUAGACA | 5386 | 110 | UGUCUAAGACUUCAGAUCC | 5384 |
| | | AU | | | UA | |
| N3T130057-01 | 113 | GAUCUGAAGUCUUAGACAA | 5387 | 114 | UUGUCUAAGACUUCAGAUC | 5385 |
| | | UC | | | CU | |
| N3T130091-01 | 181 | GACAUACCCUAGAAAAGAU | 6839 | 182 | AUCUUUUCUAGGGUAUGUC | 6837 |
| | | GA | | | UC | |

Further preferred siRNAs are listed in Table 2b:

TABLE 2b

| | SEQ | | | SEQ | | |
|---|---|---|---|---|---|---|
| Duplex | ID NO | sense strand (5′ to 3′) | Range* | ID NO | antisense strand (5′ to 3′) | Range* |
| N3T130100-01 | 199 | GACGAGGAAACUCUGAAGAA | 169 | 200 | UCUUCAGAGUUUCCUCGUCC | 167 |
| | | G | | | U | |
| N3T130001-01 | 1 | GUUGGUCACUUCUGUGCAAA | 377 | 2 | UUGCACAGAAGUGACCAACC | 375 |
| | | U | | | C | |
| N3T130008-01 | 15 | GAGUGGCAAUGUCAGGUGUU | 864 | 16 | ACACCUGACAUUGCCACUCA | 862 |
| | | A | | | U | |
| N3T130023-01 | 45 | GGCAAUGUCAGGUGUUAUAA | 868 | 46 | UAUAACACCUGACAUUGCCA | 866 |
| | | U | | | C | |
| N3T130002-01 | 3 | GGGAUUCAGAAAUGUGGAUU | 1807 | 4 | AUCCACAUUUCUGAAUCCCA | 1805 |
| | | A | | | G | |
| N3T130094-01 | 187 | GAGCGUGUGAUCAGAAUAAC | 2279 | 188 | UUAUUCUGAUCACACGCUCU | 2277 |
| | | A | | | C | |
| N3T130096-01 | 191 | GGACUUGCACAGUAAUAAAU | 3255 | 192 | UUUAUUACUGUGCAAGUCCA | 3253 |
| | | U | | | A | |
| N3T130004-01 | 7 | GACCCUCAGUGGUUUUAGAU | 3347 | 8 | UCUAAAACCACUGAGGGUCC | 3345 |
| | | C | | | A | |
| N3T130099-01 | 197 | GGUCUUUAGAUAUGAGCAGC | 3731 | 198 | CUGCUCAUAUCUAAAGACCG | 3729 |
| | | A | | | C | |
| N3T130069-01 | 137 | GGGAACUCUUAUUUAGCCAU | 3803 | 138 | UGGCUAAAUAAGAGUUCCCU | 3801 |
| | | A | | | U | |
| N3T130028-01 | 55 | GGAACUCUUAUUUAGCCAUA | 3804 | 56 | AUGGCUAAAUAAGAGUUCCC | 3802 |
| | | A | | | U | |
| N3T130034-01 | 67 | GCCAUAAUCAGAUCAGCAUC | 3818 | 68 | AUGCUGAUCUGAUUAUGGCU | 3816 |
| | | U | | | A | |
| N3T130097-01 | 193 | GGUCGUGAGGAAUUCUAUAG | 4324 | 194 | UAUAGAAUUCCUCACGACCU | 4322 |
| | | U | | | G | |
| N3T130059-01 | 117 | GUGAAUGCCACCGAGGAAUC | 4597 | 118 | AUUCCUCGGUGGCAUUCACA | 4595 |
| | | U | | | A | |
| N3T130055-01 | 109 | GGAUCUGAAGUCUUAGACAA | 5386 | 110 | UGUCUAAGACUUCAGAUCCU | 5384 |
| | | U | | | A | |
| N3T130057-01 | 113 | GAUCUGAAGUCUUAGACAAU | 5387 | 114 | UUGUCUAAGACUUCAGAUCC | 5385 |
| | | C | | | U | |
| N3T130091-01 | 181 | GACAUACCCUAGAAAAGAUG | 6839 | 182 | AUCUUUUCUAGGGUAUGUCU | 6837 |
| | | A | | | C | |

The range positions refer to the start of the sequence corresponding a position on the LRRK2 mRNA, wherein the numbering is given with respect to the LRRK2 genomic DNA (NCBI refseqID NG_011709.2; NCBI GeneID: 120892; NM_198578.4). The ranges corresponding to the mRNA regions complementary to the antisense sequences in Table 2a are hotspots where the mRNA is amenable to suppression by siRNA, so siRNAs targeting mRNA regions overlapping with these hotspot sequences are expected to be effective.

Based on their potency, 17 siRNAs are selected for further study at 1 nM, 0.1 nM, and 0.01 nM (Table 3):

TABLE 3
Dose Screens of Human LRRK2 siRNAs
in A549 Cells by Transfection

| | Average | | Average | | Average | |
|---|---|---|---|---|---|---|
| | % LRRK2 | | % LRRK2 | | % LRRK2 | |
| | mRNA | | mRNA | | mRNA | |
| | Remaining | ST | Remaining | ST | Remaining | ST |
| Duplex | (1 nM) | DEV | (0.1 nM) | DEV | (0.01 nM) | DEV |
| N3T130001- | 25.24 | 0.75 | 26.92 | 0.87 | 74.74 | 5.42 |
| 01 | | | | | | |
| N3T130002- | 24.33 | 2.50 | 27.31 | 0.69 | 68.37 | 1.66 |
| 01 | | | | | | |
| N3T130023- | 30.21 | 1.76 | 26.71 | 1.08 | 49.96 | 8.93 |
| 01 | | | | | | |
| N3T130026- | 23.58 | 0.18 | 29.10 | 0.56 | 86.56 | 0.73 |
| 01 | | | | | | |
| N3T130034- | 22.32 | 0.73 | 29.00 | 1.49 | 86.42 | 8.17 |
| 01 | | | | | | |
| N3T130035- | 20.75 | 1.40 | 25.48 | 0.77 | 70.30 | 3.17 |
| 01 | | | | | | |
| N3T130055- | 26.12 | 3.32 | 26.32 | 0.98 | 87.37 | 13.49 |
| 01 | | | | | | |
| N3T130057- | 22.43 | 3.33 | 28.90 | 1.43 | 78.20 | 0.87 |
| 01 | | | | | | |
| N3T130059- | 14.35 | 1.44 | 32.29 | 1.47 | 92.40 | 2.66 |
| 01 | | | | | | |
| N3T130069- | 20.42 | 0.07 | 43.34 | 2.65 | 85.41 | 8.85 |
| 01 | | | | | | |
| N3T130091- | 26.99 | 4.77 | 35.77 | 5.16 | 89.73 | 10.24 |
| 01 | | | | | | |
| N3T130094- | 29.06 | 0.74 | 42.12 | 1.40 | 87.86 | 5.53 |
| 01 | | | | | | |
| N3T130095- | 30.09 | 0.12 | 33.40 | 3.70 | 70.2 | 5.71 |
| 01 | | | | | | |
| N3T130096- | 21.38 | 5.09 | 26.46 | 0.45 | 59.05 | 1.12 |
| 01 | | | | | | |
| N3T130097- | 25.9 | 0.21 | 29.07 | 1.81 | 76.70 | 0.45 |
| 01 | | | | | | |
| N3T130099- | 26.16 | 4.09 | 41.40 | 1.82 | 108.89 | 4.60 |
| 01 | | | | | | |
| N3T130100- | 43.75 | 3.77 | 56.28 | 4.17 | 108.80 | 0.93 |
| 01 | | | | | | |

14 siRNA duplexes have more than 70% mRNA knockdown activity at 1 nM. 10 siRNA duplexes have more than 70% mRNA knockdown activity at 0.1 nM (Table 4):

TABLE 4
siRNA constructs having more than 70% mRNA knockdown activity at 0.1 nM

| | SEQ | sense | | SEQ | antisense | |
|---|---|---|---|---|---|---|
| Duplex | ID NO | strand (5′ to 3′) | NM_198578.4 | ID NO | strand (5′ to 3′) | NM_198578.4 |
| N3T130001-01 | 1 | GUUGGUCACUUCUGUGC | 377 | 2 | UUGCACAGAAGUGACCA | 375 |
| | | AAAU | | | ACCC | |
| N3T130002-01 | 3 | GGGAUUCAGAAAUGUGG | 1807 | 4 | AUCCACAUUUCUGAAUC | 1805 |
| | | AUUA | | | CCAG | |
| N3T130023-01 | 45 | GGCAAUGUCAGGUGUUA | 868 | 46 | UAUAACACCUGACAUUG | 866 |
| | | UAAU | | | CCAC | |
| N3T130026-01 | 51 | GCCUUAUUUUCCAAUGG | 5226 | 52 | UCCCAUUGGAAAAUAAG | 5224 |
| | | GAUU | | | GCAU | |
| N3T130034-01 | 67 | GCCAUAAUCAGAUCAGC | 3818 | 68 | AUGCUGAUCUGAUUAUG | 3816 |
| | | AUCU | | | GCUA | |
| N3T130035-01 | 69 | GAUAUGAGCAGCAAUGA | 3739 | 70 | UAUCAUUGCUGCUCAUA | 3737 |
| | | UAUU | | | UCUA | |
| N3T130055-01 | 109 | GGAUCUGAAGUCUUAGA | 5386 | 110 | UGUCUAAGACUUCAGAU | 5384 |
| | | CAAU | | | CCUA | |
| N3T130057-01 | 113 | GAUCUGAAGUCUUAGAC | 5387 | 114 | UUGUCUAAGACUUCAGA | 5385 |
| | | AAUC | | | UCCU | |
| N3T130096-01 | 191 | GGACUUGCACAGUAAUA | 3255 | 192 | UUUAUUACUGUGCAAGU | 3253 |
| | | AAUU | | | CCAA | |
| N3T130097-01 | 193 | GGUCGUGAGGAAUUCUA | 4324 | 194 | UAUAGAAUUCCUCACGA | 4322 |
| | | UAGU | | | CCUG | |

Two siRNA duplexes (N3T130023-01 and N3T130096-01) have more than 40% mRNA knockdown activity at 0.01 nM. mRNA regions overlapping with regions complementary to the antisense sequences in Table 4 are hotspots where the mRNA is amenable to suppression by siRNA.

### Example 4. siRNAs with 3′ Antisense Overhang and 5′ Antisense Blunt End

Different length variations for naked siRNAs are provided below (Table 5). These strands have a 3′ antisense overhang (i.e., 5′ end of the antisense strand in these duplexes is complementary to the 3′end of the sense strand, so the sequence is blunt at the 3′ end of the sense strand, but at the 5′ end of the sense strand, the 3′ end of the antisense strand overhangs the sense strand, e.g., by 2 nucleotides). Lengths of antisense strands range from 21-23 nucleotides; Lengths of sense strands range from 19-21 nucleotides. 3′ overhangs of 2 nucleotides in antisense strands are kept.

TABLE 5
Unmodified Human LRRK2 siRNAs

| | SEQ | sense strand | Number | | SEQ | antisense strand | Number | |
|---|---|---|---|---|---|---|---|---|
| Duplex | ID NO | (5′ to 3′) | of nt | Range* | ID NO | (5′ to 3′) | of nt | Range* |
| N3T130101- | 221 | GGCAAUGUCAGGU | 19 | 868 | 46 | UAUAACACCUGACA | 21 | 866 |
| 01 | | GUUAUA | | | | UUGCCAC | | |
| N3T130102- | 222 | GUGGCAAUGUCAG | 21 | 866 | 223 | UAUAACACCUGACA | 23 | 864 |
| 01 | | GUGUUAUA | | | | UUGCCACUC | | |
| N3T130103- | 224 | GGACUUGCACAGU | 19 | 3255 | 192 | UUUAUUACUGUGCA | 21 | 3253 |
| 01 | | AAUAAA | | | | AGUCCAA | | |
| N3T130104- | 225 | UUGGACUUGCACA | 21 | 3253 | 226 | UUUAUUACUGUGCA | 23 | 3251 |
| 01 | | GUAAUAAA | | | | AGUCCAAAU | | |
| N3T130105- | 227 | GUUGGUCACUUCU | 19 | 377 | 2 | UUGCACAGAAGUGA | 21 | 375 |
| 01 | | GUGCAA | | | | CCAACCC | | |
| N3T130106- | 228 | GGGUUGGUCACUU | 21 | 375 | 229 | UUGCACAGAAGUGA | 23 | 373 |
| 01 | | CUGUGCAA | | | | CCAACCCAC | | |
| N3T130107- | 230 | GGGAUUCAGAAAU | 19 | 1807 | 231 | UUCCACAUUUCUGA | 21 | 1805 |
| 01 | | GUGGAA | | | | AUCCCAG | | |
| N3T130108- | 232 | CUGGGAUUCAGAA | 21 | 1805 | 233 | UUCCACAUUUCUGA | 23 | 1801 |
| 01 | | AUGUGGAA | | | | AUCCCAGGA | | |
| N3T130109- | 234 | GAUAUGAGCAGCA | 19 | 3739 | 70 | UAUCAUUGCUGCUC | 21 | 3737 |
| 01 | | AUGAUA | | | | AUAUCUA | | |
| N3T130110- | 235 | UAGAUAUGAGCAG | 21 | 3737 | 236 | UAUCAUUGCUGCUC | 23 | 3735 |
| 01 | | CAAUGAUA | | | | AUAUCUAAA | | |
| N3T130111- | 237 | GGUCGUGAGGAAU | 19 | 4324 | 194 | UAUAGAAUUCCUCA | 21 | 4322 |
| 01 | | UCUAUA | | | | CGACCUG | | |
| N3T130112- | 238 | CAGGUCGUGAGGA | 21 | 4322 | 239 | UAUAGAAUUCCUCA | 23 | 4320 |
| 01 | | AUUCUAUA | | | | CGACCUGCA | | |
| N3T130113- | 240 | GAUCUGAAGUCUU | 19 | 5387 | 114 | UUGUCUAAGACUUC | 21 | 5385 |
| 01 | | AGACAA | | | | AGAUCCU | | |
| N3T130114- | 241 | AGGAUCUGAAGUC | 21 | 5385 | 242 | UUGUCUAAGACUUC | 23 | 5383 |
| 01 | | UUAGACAA | | | | AGAUCCUAC | | |
| N3T130115- | 243 | GAUCUCGUGAACA | 19 | 2429 | 244 | UCAUCUUGUUCACG | 21 | 2427 |
| 01 | | AGAUGA | | | | AGAUCCA | | |
| N3T130116- | 245 | UGGAUCUCGUGAA | 21 | 2427 | 246 | UCAUCUUGUUCACG | 23 | 2425 |
| 01 | | CAAGAUGA | | | | AGAUCCACU | | |
| N3T130117- | 247 | GGAUCUGAAGUCU | 19 | 5386 | 110 | UGUCUAAGACUUCA | 21 | 5384 |
| 01 | | UAGACA | | | | GAUCCUA | | |
| N3T130118- | 248 | UAGGAUCUGAAGU | 21 | 5384 | 249 | UGUCUAAGACUUCA | 23 | 5382 |
| 01 | | CUUAGACA | | | | GAUCCUACC | | |
| N3T130119- | 250 | GCCUUAUUUUCCA | 19 | 5226 | 52 | UCCCAUUGGAAAAU | 21 | 5224 |
| 01 | | AUGGGA | | | | AAGGCAU | | |
| N3T1300120- | 251 | AUGCCUUAUUUUC | 21 | 5224 | 252 | UCCCAUUGGAAAAU | 23 | 5222 |
| 01 | | CAAUGGGA | | | | AAGGCAUUU | | |
| N3T130121- | 253 | GCCAUAAUCAGAU | 19 | 3818 | 254 | UUGCUGAUCUGAUU | 21 | 3816 |
| 01 | | CAGCAA | | | | AUGGCUA | | |
| N3T130122- | 255 | UAGCCAUAAUCAG | 21 | 3816 | 256 | UUGCUGAUCUGAUU | 23 | 3814 |
| 01 | | AUCAGCAA | | | | AUGGCUAAA | | |
| N3T130123- | 257 | GGUGAUCAGAUAU | 19 | 2661 | 258 | UAUCUGAUAUCUGA | 21 | 2659 |
| 01 | | CAGAUA | | | | UCACCAU | | |
| N3T130124- | 259 | AUGGUGAUCAGAU | 21 | 2659 | 260 | UAUCUGAUAUCUGA | 23 | 2657 |
| 01 | | AUCAGAUA | | | | UCACCAUUC | | |

TABLE 6
In Vitro Screening of Human LRRK2 siRNAs by Transfection

| | Average % LRRK2 mRNA | |
|---|---|---|
| Duplex | Remaining (0.1 nM) | STDEV |
| N3T130101-01 | 25.9 | 4.21 |
| N3T130102-01 | 34.2 | 7.81 |
| N3T130103-01 | 28.8 | 1.80 |
| N3T130104-01 | 32.5 | 0.32 |
| N3T130105-01 | 31.6 | 0.61 |
| N3T130106-01 | 28.6 | 2.81 |
| N3T130107-01 | 37.6 | 4.08 |
| N3T130108-01 | 40.4 | 4.21 |
| N3T130109-01 | 26.1 | 0.17 |
| N3T130110-01 | 32.4 | 3.61 |
| N3T130111-01 | 34.5 | 5.87 |
| N3T130112-01 | 28.2 | 3.70 |
| N3T130113-01 | 30.7 | 5.21 |
| N3T130114-01 | 33.2 | 5.06 |
| N3T130115-01 | 35.8 | 6.01 |
| N3T130116-01 | 35.5 | 5.66 |
| N3T130117-01 | 35.8 | 1.73 |
| N3T130118-01 | 32.0 | 0.03 |
| N3T130119-01 | 46.3 | 9.06 |
| N3T130120-01 | 37.2 | 1.75 |
| N3T130121-01 | 42.5 | 4.97 |
| N3T130122-01 | 43.8 | 3.18 |
| N3T130123-01 | 48.5 | 10.47 |
| N3T130124-01 | 35.1 | 1.96 |

Human LRRK2 siRNAs with a sense strand of 19 nucleotides and an antisense strand of 21 nucleotides have similar mRNA knockdown activity to corresponding siRNAs with a sense strand of 21 nucleotides and an antisense strand of 23 nucleotides (Table 6).

### Example 5. In Vitro Evaluation of Human LRRK2 siRNAs by Free Uptake

The LRRK2 siRNAs (Table 7) are added into the Human Lung Epithelial cells A549 in a 96-well plate without transfection reagents. All the siRNAs are tested at 10 μM and 3 μM. 72 hours after free uptake, the culture medium is discarded and cells are collected for RNA extraction. Total RNA is extracted by FastPure Cell/Tissue Total RNA Isolation Kit V2 (Vazyme-RC112-01) according to the kit instructions and reversed transcribed to cDNA by HiScript III All-in-one RT SuperMix Perfect for qPCR (Vazyme-R333-01). The cDNA of the target gene is quantified by qPCR with specific primer and probe sets. GAPDH cDNA is measured as a housekeeping gene in parallel. The expression of the target gene in each test sample is determined (Table 8) by relative quantitation (RQ) using the comparative Ct (ΔΔCt) method.

TABLE 7
Human LRRK2 siRNAs for Free Uptake in A549 Cells

| | SEQ ID | | SEQ ID | |
|---|---|---|---|---|
| Duplex | NO | sense strand (5′ to 3′) | NO | antisense strand (5′ to 3′) |
| N3T130129-02 | 286 | CmsUmsUmGmUm(Uhd)UfGmUfAf | 287 | VPUmsGfsGmAmAmUfUmGmCmA |
| | | UfUmGmCmAmAmUmUmCmsCms | | mAmUmAmCfAmAfAmCmAmAm |
| | | Am | | GmsUmsGm |
| N3T130138-01 | 288 | CmsUmsUmGmUmUmUfGmUfAfU | 289 | VPUmsGfsGmAmAmUfUmGmCmA |
| | | fUmGmCmAmAmUmUmCmCmsA | | mAmUmAmCfAmAfAmCmAmAm |
| | | ms[N3T12-012-01] | | GmsUmsGm |
| N3T130145-01 | 290 | GmsUmsGmGmCmAmAfUmGfUfC | 291 | VPUmsAfsUmAmAmCfAmCmCmU |
| | | fAmGmGmUmGmUmUmAmUmsA | | mGmAmCmAfUmUfGmCmCmAm |
| | | ms[N3T12-012-01] | | CmsUmsCm |
| N3T130146-01 | 292 | UmsUmsGmGmAmCmUfUmGfCfA | 293 | VPUmsUfsUmAmUmUfAmCmUmG |
| | | fCmAmGmUmAmAmUmAmAmsA | | mUmGmCmAfAmGfUmCmCmAm |
| | | ms[N3T12-012-01] | | AmsAmsUm |
| N3T130147-01 | 294 | GmsGmsGmUmUmGmGfUmCfAfC | 295 | VPUms UfsGmCmAmCfAmGmAmA |
| | | fUmUmCmUmGmUmGmCmAmsA | | mGmUmGmAfCmCfAmAmCmCmC |
| | | ms[N3T12-012-01] | | msAmsCm |
| N3T130148-01 | 296 | CmsAmsGmGmUmCmGfUmGfAfG | 297 | VPUmsAfsUmAmGmAfAmUmUmC |
| | | fGmAmAmUmUmCmUmAmUmsA | | mCmUmCmAfCmGfAmCmCmUmG |
| | | ms[N3T12-012-01] | | msCmsAm |
| N3T130149-01 | 298 | UmsAmsGmAmUmAmUfGmAfGfC | 299 | VPUms AfsUmCmAmUfUmGmCmU |
| | | fAmGmCmAmAmUmGmAmUmsA | | mGmCmUmCfAmUfAmUmCmUm |
| | | ms[N3T12-012-01] | | AmsAmsAm |
| N3T130150-01 | 300 | CmsUmsGmGmGmAmUfUmCfAfG | 301 | VPUms UfsCmCmAmCfAmUmUmU |
| | | fAmAmAmUmGmUmGmGmAmsA | | mCmUmGmAfAmUfCmCmCmAm |
| | | ms[N3T12-012-01] | | GmsGmsAm |
| N3T130151-01 | 302 | AmsGmsGmAmUmCmUfGmAfAfG | 303 | VPUms UfsGmUmCmUfAmAmGmA |
| | | fUmCmUmUmAmGmAmCmAmsA | | mCmUmUmCfAmGfAmUmCmCm |
| | | ms[N3T12-012-01] | | UmsAmsCm |
| N3T130152-01 | 304 | UmsGmsGmAmUmCmUfCmGfUfG | 305 | VPUmsCfsAmUmCmUfUmGmUmU |
| | | fAmAmCmAmAmGmAmUmGmsA | | mCmAmCmGfAmGfAmUmCmCm |
| | | ms[N3T12-012-01] | | AmsCmsUm |
| N3T130153-01 | 306 | UmsAmsGmCmCmAmUfAmAfUfCf | 307 | VPUms UfsGmCmUmGfAmUmCmU |
| | | AmGmAmUmCmAmGmCmAmsAm | | mGmAmUmUfAmUfGmGmCmUm |
| | | s[N3T12-012-01] | | AmsAmsAm |
| N3T130154-01 | 308 | AmsUmsGmGmUmGmAfUmCfAfG | 309 | VPUms AfsUmCmUmGfAmUmAmU |
| | | fAmUmAmUmCmAmGmAmUmsA | | mCmUmGmAfUmCfAmCmCmAm |
| | | ms[N3T12-012-01] | | UmsUmsCm |

[N3T12-012-01]: 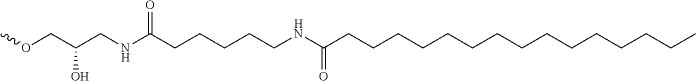
[VPUm]: 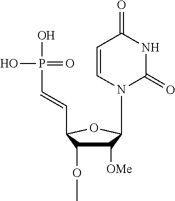
Am, Cm, Gm, Um indicate 2′-O-methyl (2′-OMe) sugar modifications, respectively, to adenosine, cytidine, guanosine and uridine;
Af, Cf Gf, Uf indicate 2′-deoxy-2′-fluoro (2′-F) sugar modifications, respectively, to adenosine, cytidine, guanosine and uridine;
s indicates phosphorothioate (PS) linkage.

TABLE 8
In Vitro Evaluation of Human LRRK2
siRNAs in A549 Cells by Free Uptake

| | Average % | | Average % | |
|---|---|---|---|---|
| | LRRK2 mRNA | | LRRK2 mRNA | |
| | Remaining | | Remaining | |
| Duplex | (10 μM) | STDEV | (3 μM) | STDEV |
| N3T130129-02 | 69.6 | 5.8 | 97.2 | 8.9 |
| N3T130138-01 | 33.9 | 2.0 | 46.3 | 2.0 |
| N3T130145-01 | 87.8 | 5.7 | 101.3 | 6.8 |
| N3T130146-01 | 32.5 | 4.0 | 51.3 | 2.5 |
| N3T130147-01 | 56.8 | 2.6 | 75.5 | 12.4 |
| N3T130148-01 | 32.3 | 0.8 | 44.0 | 8.7 |
| N3T130149-01 | 45.1 | 7.3 | 62.1 | 2.4 |
| N3T130150-01 | 68.7 | 11.8 | 81.0 | 11.2 |
| N3T130151-01 | 52.3 | 0.2 | 74.5 | 8.4 |
| N3T130152-01 | 98.6 | 8.1 | 90.1 | 27.7 |
| N3T130153-01 | 68.8 | 34.2 | 83.3 | 6.3 |
| N3T130154-01 | 75.5 | 0.0 | 63.5 | 17.2 |

Human LRRK2 siRNAs N3T130138-01, N3T130146-01, N3T130148-01 and N3T130149-01 have more than 50% mRNA knockdown activity at 10 μM. Human LRRK2 siRNAs N3T130138-01, N3T130146-01 and N3T130148-01 has more than 40% mRNA knockdown activity at 3 μM.

### Example 6. Synthesis of Lipophilic Monomers

The lipophilic monomer containing a lipophilic moiety is located on one or more terminal positions on at least one strand of the oligonucleotide. Lipophilic monomers are synthesized to introduce lipophilic ligands at various locations of siRNAs (terminal and/or internal positions) as solid support or phosphoramidites. A variety of lipids can be conjugated using methods as shown in the schemes below (e.g., Schemes 11-20 for general procedures), and the resulting building block phosphoramidites can be incorporated into siRNAs.

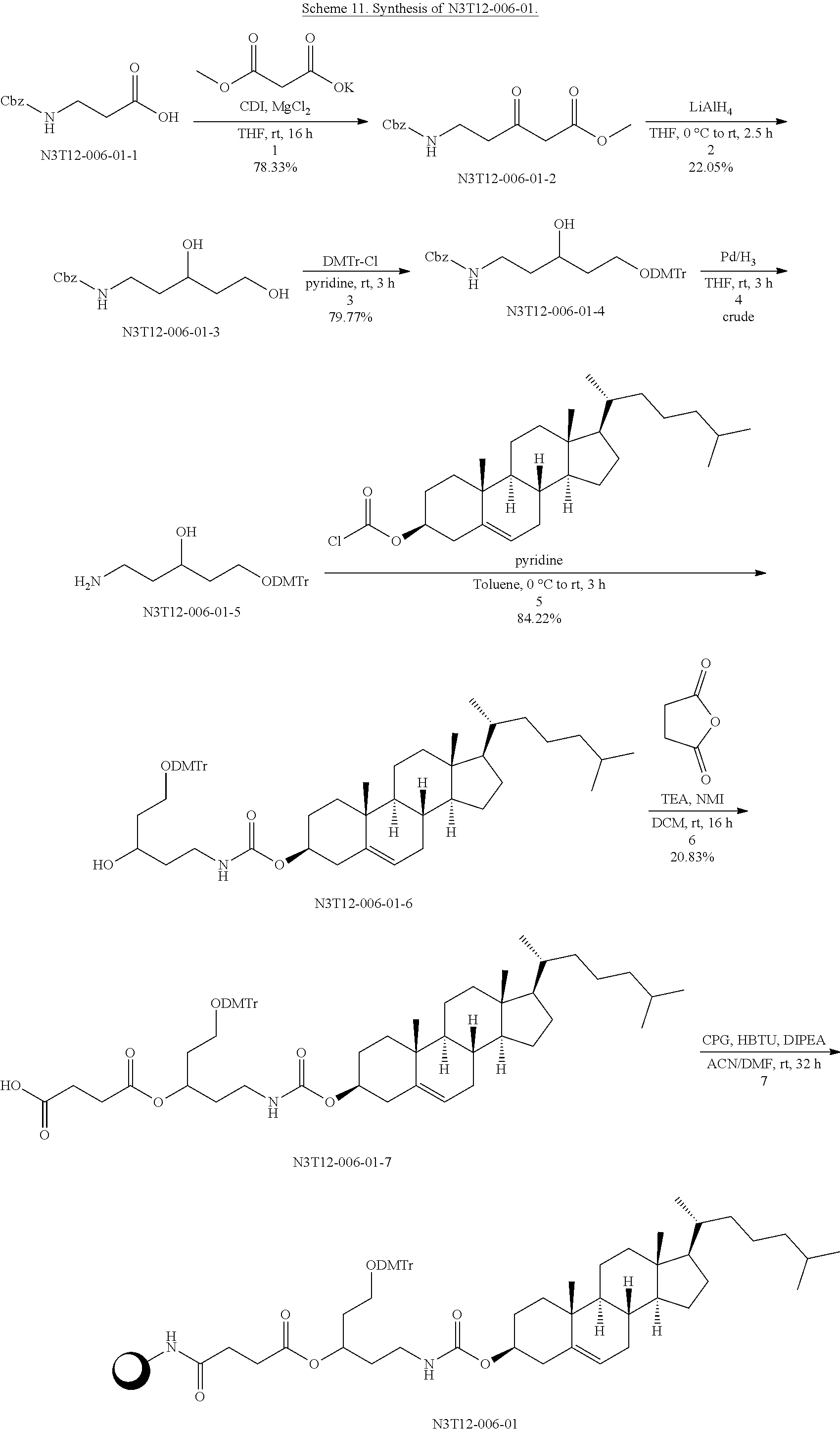

N3T12-006-01-2: A suspension of magnesium chloride (4.5 g, 46.589 mmol) and [(3-methoxy-1,3-dioxopropyl)oxy]potassium (10.5 g, 67.195 mmol) in tetrahydrofuran (200 mL) is combined with a prepared solution of CDI (8.7 g, 53.756 mmol) and 3-{[(benzyloxy) carbonyl]amino}propanoic acid (10.0 g, 44.797 mmol) in tetrahydrofuran (150 mL). The mixture is stirred at room temperature for 16 h. The reaction solution is poured into water (300 mL), extracted with EA (150 mL×3). The organic phase is concentrated to dryness. The residue is purified by column chromatography (EA/PE=0-50%) to give N3T12-006-01-2 (9.8 g, 35.089 mmol, 78.33% yield) as a colorless oil. LCMS: m/z 280.2 [(M+H)⁺]. ¹H NMR (400 MHz, CDCl3) δ 7.39-7.29 (m, 5H), 5.20 (s, 1H), 5.13-5.01 (m, 2H), 3.73 (s, 3H), 3.49-3.42 (m, 4H), 2.81 (t, J=5.6 Hz, 2H).

N3T12-006-01-3: To the solution of N3T12-006-01-2 (9.8 g, 35.089 mmol) in tetrahydrofuran (100 mL) is added LiAlH₄ (71.6 mL, 1 M in THF) slowly at 0° C. for 30 mins, then the temperature of reaction solution is warmed to room temperature, and stirred for 2 h. The reaction solution is poured into ammonium chloride aqueous solution (300 mL), extracted with EA (300 mL×4). The organic phase is concentrated in vacuo. The residue is purified by column chromatography (MeOH/DCM=0-7%) to give N3T12-006-01-3 (2.0 g, 7.896 mmol, 22.05% yield) as a colorless oil. LCMS: m/z 254.2 [(M+H)⁺]. 1H NMR (400 MHz, DMSO-d₆) δ 7.38-7.30 (m, 5H), 7.16-7.14 (m, 1H), 5.00 (s, 2H), 4.37 (d, J=5.4 Hz, 1H), 4.31 (t, J=5.2 Hz, 1H), 3.59-3.54 (m, 1H), 3.50-3.45 (m, 2H), 3.14-2.99 (m, 2H), 1.57-1.36 (m, 4H).

N3T12-006-01-4: To the solution of N3T12-006-01-3 (2.0 g, 7.896 mmol) is added 1-[chloro(4-methoxyphenyl)phenylmethyl]-4-methoxybenzene (3.2 g, 9.475 mmol) at 0° C. for 10 mins. The temperature of the mixture solution is warmed to room temperature, and stirred at this temperature for 3 h. TLC showed that SM is consumed. The reaction solution is poured into water (100 mL), adjusted to pH=4 with aqueous citric acid (10%), extracted with DCM (70 mL×2). The organic phase is concentrated in vacuo. The residue is purified by column chromatography [(EA+1% TEA)/PE=0-45%] to give N3T12-006-01-4 (3.5 g, 6.299 mmol, 79.77% yield) as a pale yellow syrup. ¹H NMR (400 MHz, DMSO-d₆) δ 7.39-7.15 (m, 15H), 6.88 (d, J=8.8 Hz, 4H), 4.99 (s, 2H), 4.35 (d, J=5.6 Hz, 1H), 3.73 (s, 6H), 3.66-3.53 (m, 1H), 3.14-2.97 (m, 4H), 1.68-1.34 (m, 4H).

N3T12-006-01-5: To the solution of N3T12-006-01-4 (2.5 g, 4.499 mmol) in tetrahydrofuran (25 mL) is added Pd/C (0.5 g, 4.499 mmol). The reaction solution is stirred at room temperature for 3 h under H₂. TLC showed SM is consumed. The reaction solution is filtered, the filtrates is concentrated in vacuo to give N3T12-006-01-5 (1.8 g, 4.270 mmol, 94.91% yield) as a dark oil, which is used for next step without further purification.

N3T12-006-01-6: To the solution of N3T12-006-01-5 (1.8 g, 4.270 mmol) in pyridine (20 mL) is added (1R,3aS,3bS,7S,9aR,9bS,11aR)-9a,11a-dimethyl-1-[(2R)-6-methylhept-2-yl]-2,3,3a,3b,4,6,7,8,9,9a,9b,10,11,11a-tetradecahydro-1H-cyclopenta [1,2-i]phenanthren-7-yl chloromethanoate (2.0 g, 4.484 mmol) in toulene (2 mL) at 0° C. for 10 mins. The mixture is stirred at room temperature for 3 h. TLC showed that SM is consumed, and a new point formed. The reaction solution is poured into water (150 mL), adjusted to pH=4 with aqueous citric acid (10%), extracted with EA (100 mL×3). The organic phase is concentrated in vacuo. The residue is purified by column chromatography [(EA+1% TEA)/PE=0-35%] to give N3T12-006-01-6 (3.0 g, 3.596 mmol, 84.22% yield) as a pale yellow resin. ¹H NMR (400 MHz, DMSO-d₆) δ 7.38-7.35 (m, 2H), 7.30 (t, J=7.6 Hz, 2H), 7.24-7.19 (m, 5H), 6.96-6.94 (m, 1H), 6.87 (d, J=8.8 Hz, 4H), 5.30 (s, 1H), 4.33-4.26 (m, 2H), 3.73 (s, 6H), 3.64-3.52 (m, 1H), 3.07-2.92 (m, 4H), 2.31-2.13 (m, 2H), 1.98-1.87 (m, 2H), 1.85-1.73 (m, 3H), 1.68-1.57 (m, 2H), 1.56-1.43 (m, 6H), 1.43-1.20 (m, 8H), 1.15-0.92 (m, 12H), 0.90 (d, J=6.5 Hz, 3H), 0.84 (dd, J=6.6, 1.8 Hz, 6H), 0.65 (s, 3H).

N3T12-006-01-7: To N3T12-006-01-6 (3.0 g, 3.596 mmol) are added tetrahydrofuran-2,5-dione (1.1 g, 10.789 mmol), 2-[ethyl(2-hydroxyethyl)amino]ethan-1-ol (1.07 mL, 10.789 mmol) and NMI (295 mg, 3.596 mmol) in DCM (10 mL). The reaction solution is stirred at room temperature for 16 h. TLC showed that SM is consumed. The reaction solution is concentrated in vacuo, the residue is purified by column chromatography [(EA+1% MeOH)/DCM=0-5%] to give N3T12-006-01-7 (700 mg, 0.749 mmol, 20.83% yield) as an off-white solid. ¹H NMR (400 MHz, DMSO-d₆) δ 12.20 (s, 1H), 7.39-7.26 (m, 4H), 7.21 (d, J=8.6 Hz, 5H), 6.97 (t, J=5.8 Hz, 1H), 6.88 (d, J=15.2 Hz, 4H), 5.37-5.24 (m, 1H), 5.02-4.92 (m, 1H), 4.36-4.23 (m, 1H), 3.73 (s, 6H), 3.02-2.91 (m, 4H), 2.44-2.15 (m, 6H), 2.05-1.92 (m, 2H), 1.83-1.67 (m, 5H), 1.63-1.50 (m, 7H), 1.45-1.22 (m, 7H), 1.17-1.06 (m, 5H), 1.04-0.93 (m, 7H), 0.92-0.83 (m, 9H), 0.65 (s, 3H).

N3T12-006-01: To the solution of N3T12-006-01-7 (90 mg, 0.097 mmol) in the mixture solution of ACN (6 mL) and DMF (3 mL) are added DIPEA (25 mg, 0.194 mmol) and HBTU (55 mg, 0.145 mmol) in a centrifugal tube. CPG (1.0 g, 180 μmol/g) is added to the mixture solution. The tube is placed on a wrist-action shaker and shaken (200 r/min) at 25° C. for 16 h. The reaction solution is filtered, filter cake is washed with DCM (30 mL×3), ACN (30 mL×3) and n-hexane (30 mL×3), respectively. After drying the filter cake in vacuo, the loading value of CPG is measured (CPG loading value=86.8 umol/g). To the mixture of CPG in ACN (5 mL) are added NMI (8 mg), pyridine (80 mg), DMAP (9 mg) and acetic anhydride (50 mg) in a centrifugal tube. The tube is placed on a wrist-action shaker and shaken (200 r/min) at 25° C. for 16 h. The reaction solution is filtered, filter cake is washed with ACN (30 mL×3). The filter cake is dried in vacuo to give N3T12-006-01 (457.20 mg) as a white solid.

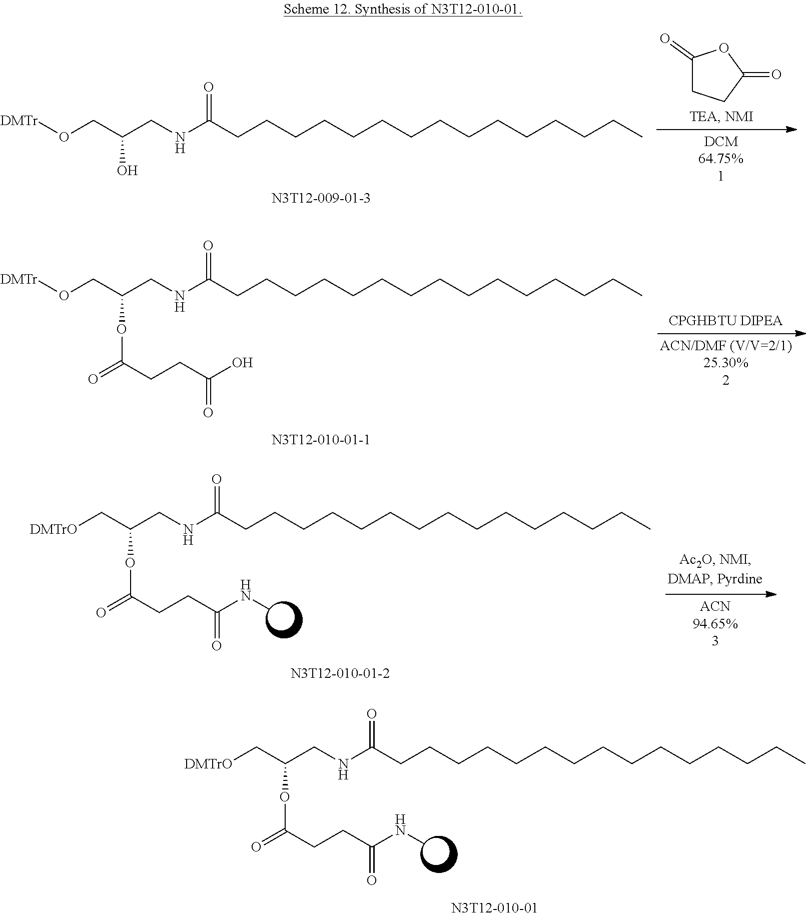

N3T12-010-01-1: To a solution of N3T12-009-01-3 (200 mg, 0.317 mmol) in DCM (4 mL) is added NMI (29 mg, 0.348 mmol) and TEA (96 mg, 0.950 mmol). Finally, add tetrahydrofuran-2,5-dione (159 mg, 1.583 mmol). The reaction is stirred for 16 h at rt and concentrated to give a residue which is purified by silica gel column chromatography (SiO₂, DCM:MeOH=93:7) to give N3T12-010-01-1 (375 mg, 0.512 mmol, 64.75% yield) as a yellow solid. LCMS: m/z 754.5 [(M+Na)⁺]. ¹H NMR (400 MHz, DMSO) δ 12.24 (s, 1H), 7.76 (t, J=6.0 Hz, 1H), 7.54-7.09 (m, 9H), 6.90-6.86 (m, 4H), 5.07-4.93 (m, 1H), 3.73 (s, 6H), 3.30-3.27 (m, 1H), 3.18-3.09 (m, 1H), 3.08-3.00 (m, 2H), 2.57-2.54 (m, 2H), 2.49-2.42 (m, 2H), 1.96 (t, J=7.4 Hz, 2H), 1.41-1.34 (m, 2H), 1.26-1.17 (m, 24H), 0.87-0.83 (m, 3H).

N3T12-010-01-2: To a solution of N3T12-010-01-1 (60 mg, 0.082 mmol) in N,N-dimethylmethanamide (6 mL) and ACN (12 mL) is added DIPEA (36 mg, 0.279 mmol) and CPG (600 mg, 0.126 mmol), finally, add HBTU (80 mg, 0.211 mmol). Shake bed reaction is carried out at 25° C. at a speed of 220 revolutions per minute. The reaction is stirred for 16 h, the filter cake is filtered and rinsed with DCM 3 times, 30 ml each time, acetonitrile 3 times, 30 ml each time, and n-hexane 3 times. The filter cake is dried with a vacuum oil pump for 2 h to give N3T12-010-01-2 as a white solid.

N3T12-010-01: To a solution of Ac₂O (94 mg, 0.917 mmol) and pyridine (145 mg, 1.833 mmol), 4-(dimethylamino)pyridine (3.5 mg, 0.028 mmol), NMI (2.5 mg, 0.028 mmol) in ACN (9 mL) is added N3T12-010-01-2 (500 mg, 0.090 mmol). Shake bed reaction is carried out at 25° C. at a speed of 220 revolutions per minute. The reaction is stirred for 16 h at rt under N₂ atmosphere. The filter cake is filtered and rinsed with acetonitrile 3 times, 30 ml each time, The filter cake is dried with a vacuum oil pump for 2 h to give N3T12-010-01 (512.31 mg, 30.170 μmol, 94.65% yield, loading value=58.89 umol/g) as a light yellow solid.

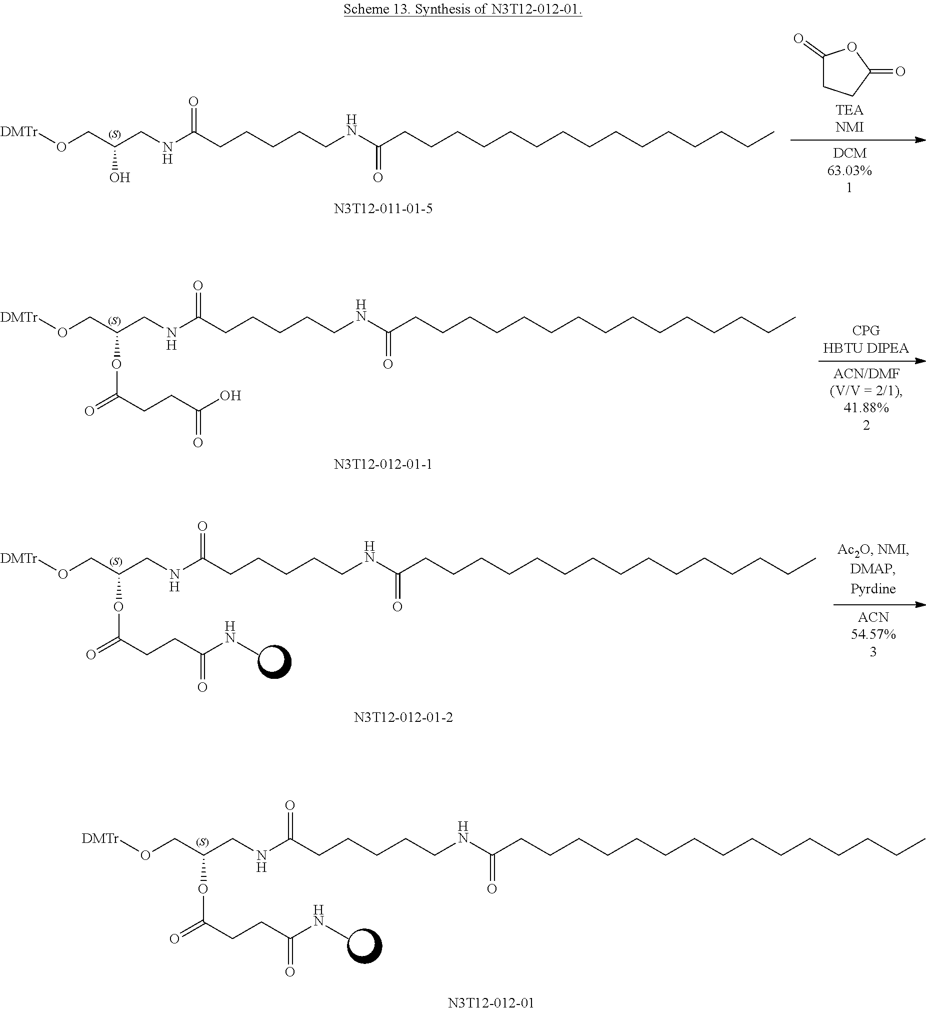

N3T12-012-01-1: To a solution of N3T12-01-01-5 (200 mg, 0.268 mmol) in DCM (2 mL) and N,N-dimethylmethanamide (2 mL) is added NMI (25 mg, 0.295 mmol) and TEA (82 mg, 0.805 mmol). Finally, add tetrahydrofuran-2,5-dione (54 mg, 0.537 mmol). The reaction is stirred for 16 hr at rt. With ice-bath cooling, the reaction is quenched with water and extracted with DCM (200 mL). The organic layer is washed with water and brine, dried over Na₂SO₄ and concentrated to give a residue which is purified by silica gel column chromatography (SiO₂, DCM:MeOH=94:6) to give N3T12-012-01-1 (143 mg, 0.169 mmol, 63.03% yield) as a colorless oil. LCMS: m/z 868.7 [(M+Na)⁺]. ¹H NMR (400 MHz, DMSO-d₆) δ 7.81-7.75 (m, 2H), 7.43-7.35 (m, 4H), 7.25-7.14 (m, 5H), 6.88 (d, J=8.9 Hz, 4H), 5.07-4.94 (m, 1H), 3.74 (s, 6H), 3.21-2.87 (m, 6H), 2.58-2.55 (m, 4H), 2.06-1.91 (m, 4H), 1.41-1.16 (m, 32H), 0.85 (t, J=6.7 Hz, 3H).

N3T12-012-01-2: To a solution of N3T12-012-01-1 (143 mg, 0.169 mmol) in N,N-dimethylmethanamide (3 mL) and ACN (6 mL) is added CPG (500 mg, 0.090 mmol) and DIPEA (19.39 mg, 0.150 mmol), finally,add HBTU (42.67 mg, 0.112 mmol). Shake bed reaction is carried out at 25° C. at a speed of 220 revolutions per minute. The reaction is stirred for 16 h, the filter cake is filtered and rinsed with DCM 3 times, 30 ml each time, acetonitrile 3 times, 30 ml each time, and n-hexane 3 times. The filter cake is dried with a vacuum oil pump for 2 h to give N3T12-012-01-2.

N3T12-012-01: To a solution of Ac₂O (70.44 mg, 0.690 mmol) and Pyridine (109.16 mg, 1.380 mmol),4-(dimethylamino)pyridine (2.53 mg, 0.021 mmol), NMI (1.70 mg, 0.021 mmol) in ACN (9 mL) is added N3T12-012-01-2 (463 mg, 0.069 mmol). Shake bed reaction is carried out at 25° C. at a speed of 220 revolutions per minute. The reaction is stirred for 16 hr at rt under N₂ atmosphere. The filter cake is filtered and rinsed with acetonitrile 3 times, 30 ml each time, The filter cake is dried with a vacuum oil pump for 2 h to give N3T12-012-01 (383 mg, 20.567 μmol, 54.57% yield, loading value=53.7 umol/g).

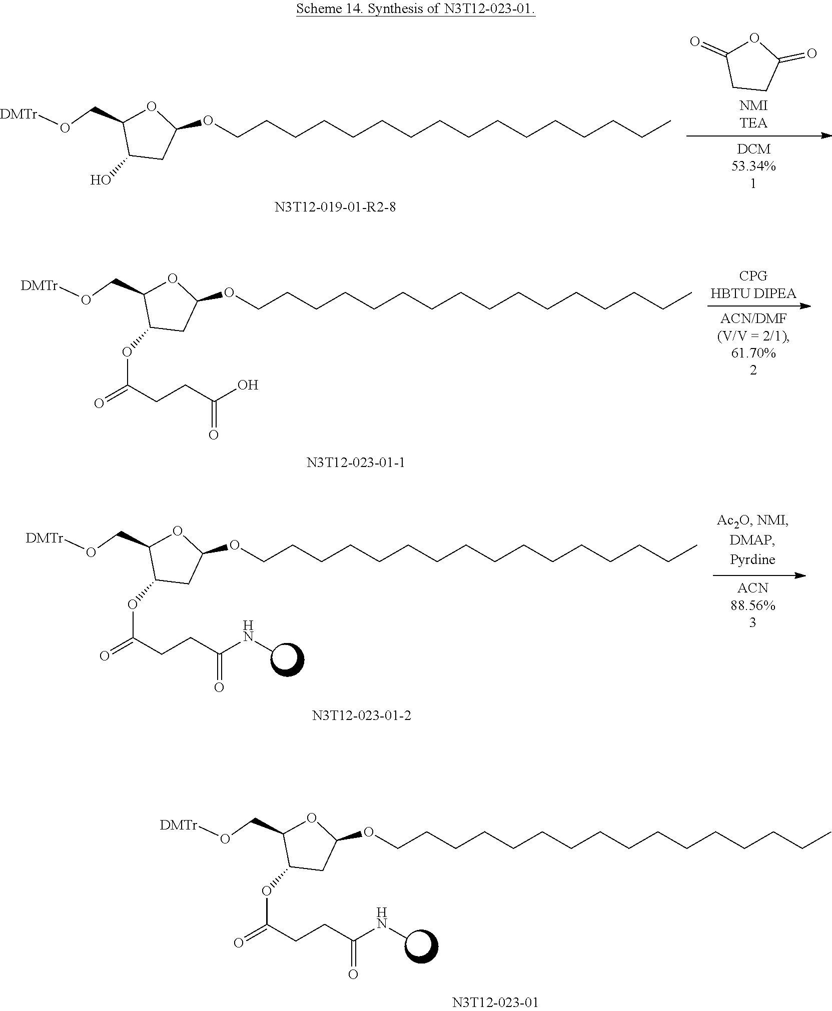

N3T12-023-01-1: To a solution of N3T12-019-01-R₂-6 (200 mg, 0.291 mmol) in DCM (4 mL) is added NMI (26 mg, 0.320 mmol) and TEA (88 mg, 0.872 mmol). Finally, added tetrahydrofuran-2,5-dione (60.56 mg, 0.605 mmol). The reaction is stirred for 16 hr at rt, concentrated to give a residue which is purified by silica gel column chromatography (SiO₂, DCM:MeOH=93:7) to give N3T12-023-01-1 (123 mg, 0.162 mmol, 53.34% yield) as a yellow oil. ¹H NMR (400 MHz, DMSO-d₆) δ 12.20 (s, 1H), 7.46-7.15 (m, 9H), 6.89-6.85 (m, 4H), 5.25-5.04 (m, 2H), 4.06 (M, 1H), 3.73 (d, J=2.4 Hz, 6H), 3.57-3.44 (m, 2H), 3.29-3.21 (m, 2H), 3.13 (dd, J=9.6, 5.8 Hz, 1H), 3.00 (dd, J=9.6, 5.4 Hz, 1H), 2.49-2.44 (m, 2H), 2.21-1.99 (m, 2H), 1.34 (t, J=6.2 Hz, 2H), 1.30-1.09 (m, 26H), 0.90-0.80 (m, 3H).

N3T12-023-01-2: To a solution of N3T12-023-01-1 (123 mg, 0.162 mmol) in N,N-dimethylmethanamide (3 mL) and ACN (6 mL) is added CPG (600 mg, 0.108 mmol) and DIPEA (42 mg, 0.324 mmol), finally, added HBTU (92.16 mg, 0.243 mmol). Shake bed reaction is carried out at 25° C. at a speed of 220 revolutions per minute. The reaction is stirred for 16 h, the filter cake is filtered and rinsed with DCM 3 times, 30 ml each time, acetonitrile 3 times, 30 ml each time, and n-hexane 3 times. The filter cake is dried with a vacuum oil pump for 2 h to give N3T12-023-01-2 as a white solid.

N3T12-023-01: To a solution of Ac₂O (43 mg, 0.420 mmol) and pyridine (100 mg, 1.260 mmol), 4-(dimethylamino)pyridine (1.5 mg, 0.013 mmol), NMI (1.1 mg, 0.013 mmol) in ACN (9 mL) is added N3T12-023-01-2 (600.00 mg, 0.042 mmol). Shake bed reaction is carried out at 25° C. at a speed of 220 revolutions per minute. The reaction is stirred for 16 h at rt under N₂ atmosphere. The filter cake is filtered and rinsed with acetonitrile 3 times, 30 ml each time, The filter cake is dried with a vacuum oil pump for 2 h to give N3T12-023-01 (554.90 mg,59.019 μmol,88.56% yield, loading value=106.36 umol/g) as a white solid.

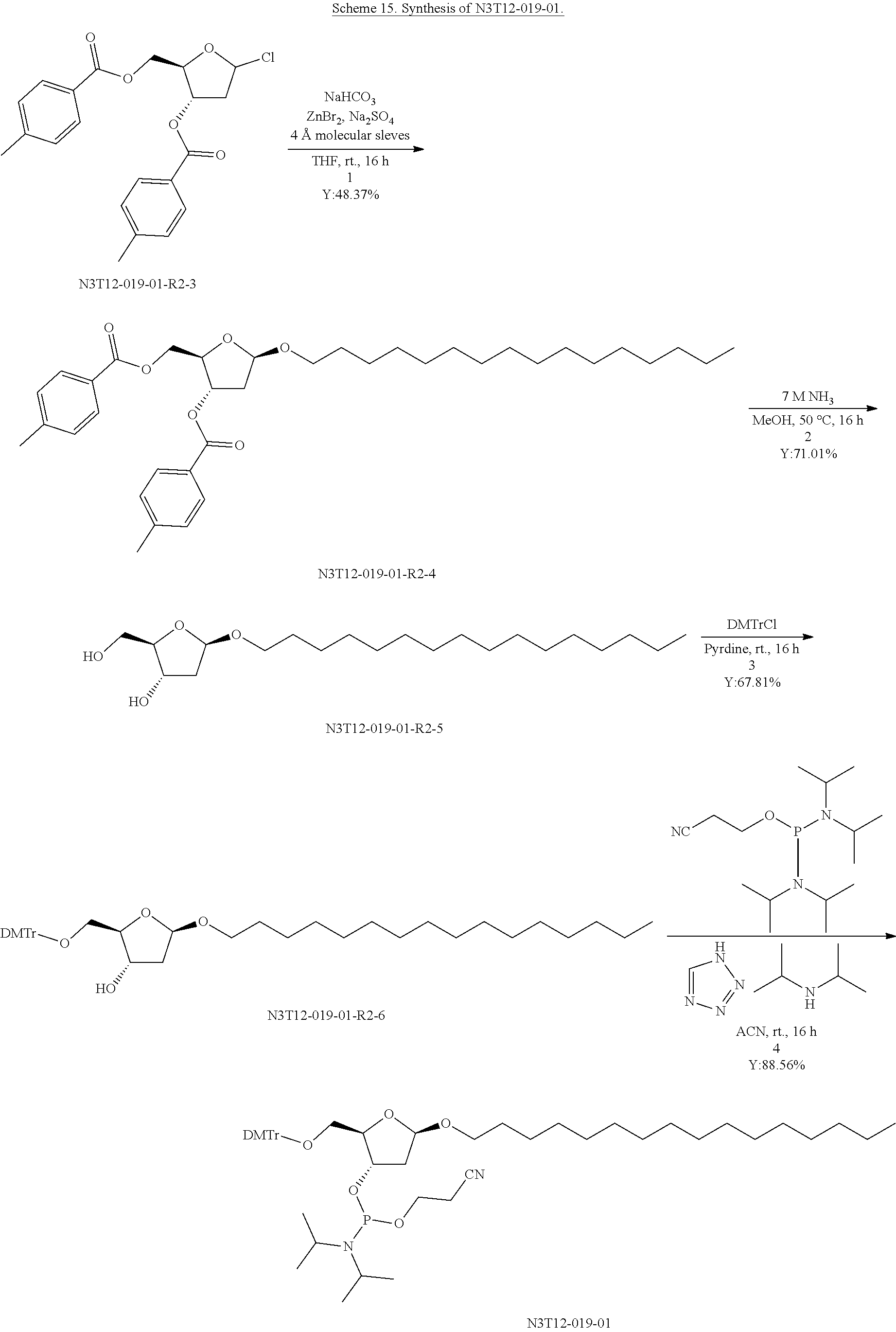

N3T12-019-01-R₂-4: To a solution of N3T12-019-01-R₂-3 (5.0 g, 12.859 mmol) in tetrahydrofuran (50 mL) is added hexadecan-1-ol (5.7 mL, 19.288 mmol), sodium bicarbonate (2.16 g, 25.718 mmol), molecular sieves (5 g) and zinc dibromide (0.87 g, 3.858 mmol). The reaction is stirred for 16 h at rt under N₂ atmosphere. The mixture is filtered and the filtrate is concentrated in vacuo. The residue is purified by flash column chromatography (eluting with 0%-15% EA in PE) to afford N3T12-019-01-R₂-4 (3.7 g, 6.220 mmol, 48.37% yield) as a white solid. ¹H NMR (400 MHz, CD₃OD-d4) δ 7.97-7.92 (m, 2H), 7.91-7.86 (m, 2H), 7.31-7.23 (m, 4H), 5.58-5.55 (m, 1H), 5.33 (dd, J=5.4, 2.4 Hz, 1H), 4.55 (dd, J=10.6, 4.6 Hz, 1H), 4.49-4.45 (m, 1H), 4.44-4.39 (m, 1H), 3.70 (dt, J=9.4, 6.6 Hz, 1H), 3.38 (dt, J=9.4, 6.6 Hz, 1H), 2.54-2.48 (m, 1H), 2.42-2.34 (m, 7H), 1.54-1.44 (m, 2H), 1.27-1.23 (m, 26H), 0.89 (t, J=6.8 Hz, 3H).

N3T12-019-01-R2-5: N3T12-019-01-R2-4 (2.8 g, 0.005 mol) in sealed tube is added NH₃ (15 mL, 7 M in MeOH). The resulting mixture is stirred for overnight at 50° C. After the reaction is completed, the solvent is evaporated to dryness under reduced pressure. The residue is purified by column chromatography over silica gel (eluting with 0%-7% MeOH in DCM) to give N3T12-019-01-R2-5 (1.2 g, 3.347 mmol, 71.01% yield) as a white solid. ¹H NMR (400 MHz, DMSO-d₆) δ 5.06 (dd, J=5.2, 2.6 Hz, 1H), 4.94 (d, J=4.8 Hz, 1H), 4.56 (t, J=5.6 Hz, 1H), 4.14-4.07 (m, 1H), 3.69 (td, J=6.0, 3.6 Hz, 1H), 3.56 (dt, J=9.4, 6.6 Hz, 1H), 3.41-3.29 (m, 2H), 3.28-3.20 (m, 1H), 2.01-1.93 (m, 1H), 1.91-1.84 (m, 1H), 1.47-1.39 (m, 2H), 1.30-1.18 (m, 26H), 0.85 (t, J=6.8 Hz, 3H).

N3T12-019-01-R2-6: To a solution of N3T12-019-01-R2-5 (1.2 g, 0.003 mol) in pyridine (10 mL) is added DMTrCl (1.70 g, 0.005 mol) in 10 mL THF. The resulting mixture is stirred for overnight at rt. Then, water (10 mL) is added to quench the reaction and evaporated to dryness under reduced pressure. The residue is purified by column chromatography over silica gel (eluting with 0%-50% EA in PE) to give N3T12-019-01-R2-6 (1.5 g, 2.269 mmol, 67.81% yield) as a yellow oil. ¹H NMR (400 MHz, DMSO-d₆) δ 7.42 (d, J=7.4 Hz, 2H), 7.32-7.25 (m, 6H), 7.24-7.18 (m, 1H), 6.87 (d, J=8.2 Hz, 4H), 5.07 (dd, J=5.0, 1.8 Hz, 1H), 5.05 (d, J=5.2 Hz, 1H), 4.12-4.06 (m, 1H), 3.85 (dd, J=10.2, 4.8 Hz, 1H), 3.73 (s, 6H), 3.53 (dt, J=9.0, 6.6 Hz, 1H), 3.25 (dt, J=9.2, 6.4 Hz, 1H), 3.04 (dd, J=9.6, 4.2 Hz, 1H), 2.98-2.92 (m, 1H), 1.96 (ddd, J=12.6, 6.6, 1.6 Hz, 1H), 1.90-1.83 (m, 1H), 1.38-1.10 (m, 28H), 0.84 (t, J=6.8 Hz, 3H).

N3T12-019-01: To a solution of N3T12-019-01-R2-6 (1.3 g, 0.002 mol) and di(prop-2-yl) ammonium 1,2,3,4-tetraazol-1-ide (0.51 g, 0.003 mol) in ACN (4 mL) is added 3-{[2,6-dimethyl-3,5-di(prop-2-yl)-3,5-diaza-4-phosphahept-4-yl]oxy}propanenitrile (1.19 g, 0.004 mol) in ACN (2 mL). The mixture is stirred at rt for overnight in the sealed tube. After the reaction is completed, the mixture is poured into a saturated sodium bicarbonate solution (100 mL). Then, the solution is extracted with DCM (100*3). The organic phase is dried with anhydrous sodium sulfate, filtrated and evaporated to dryness under reduced pressure. The residue is purified by reversed phase column (100% ACN) to give N3T12-019-01 (1.5 g, 1.742 mmol, 88.56%) as a colorless oil. LCMS (TM hydrolysate): m/z 800.6 [(M+Na)⁺]. ¹H NMR (400 MHz, DMSO-d₆) δ 7.42 (dd, J=7.0, 5.0 Hz, 2H), 7.32-7.25 (m, 6H), 7.21 (dd, J=8.2, 6.0 Hz, 1H), 6.86 (dd, J=8.8, 3.8 Hz, 4H), 5.16-5.11 (m, 1H), 4.44-4.29 (m, 1H), 4.07-3.94 (m, 1H), 3.72 (s, 6H), 3.70-3.63 (m, 1H), 3.62-3.42 (m, 4H), 3.31-3.24 (m, 1H), 3.19-3.06 (m, 1H), 3.01-2.95 (m, 1H), 2.73 (t, J=5.8 Hz, 1H), 2.63 (t, J=5.8 Hz, 1H), 2.10-2.00 (m, 1H), 1.39-1.31 (m, 2H), 1.40-1.05 (m, 36H), 0.97 (d, J=6.6 Hz, 3H), 0.84 (t, J=6.8 Hz, 3H). ³¹P NMR (162 MHz, DMSO) δ 147.40 (s), 147.04 (s).

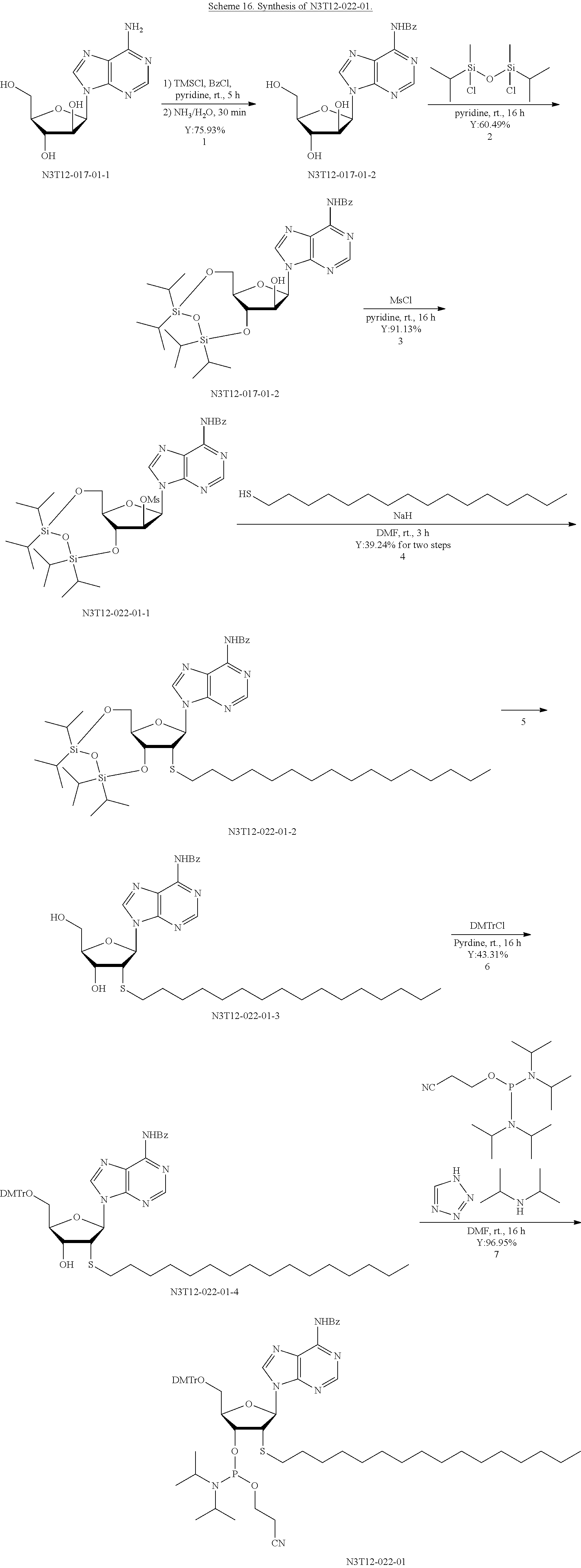

N3T12-017-01-2: To a solution of N3T12-017-01-1 (20.0 g, 0.075 mol) in pyridine (200 mL) is added TMSCl (32.6 g, 0.299 mol) and the mixture is stirred at rt for 1 h whereupon BzCl (12.7 g, 0.090 mol) is added dropwise. The resulting mixture is stirred for 4 h at rt, whereupon water (20 mL) is added. After stirring for 5 min at rt, aqueous ammonia (180 mL) is added and the resulting mixture is stirred for 30 min at rt and then evaporated to dryness under reduced pressure. The residue is purified by column chromatography over silica gel (eluting with 0%-7% MeOH in DCM) to give N3T12-017-01-2 (21.1 g, 56.820 mmol, 75.93% yield) as a white solid. LCMS: m/z 372.3 [(M+H)⁺].

N3T12-017-01-3: To a solution of N3T12-017-01-2 (10.5 g, 0.027 mol) in pyridine (200 mL) is added 3,5-dichloro-2,6-dimethyl-3,5-di(prop-2-yl)-3,5-disila-4-oxaheptane (17.0 g, 0.054 mol) dropwise. The mixture is stirred at room temperature (rt) for overnight. After the reaction is completed, water (10 mL) is added to quench the reaction and evaporated to dryness under reduced pressure. The residue is purified by column chromatography over silica gel (eluting with 0%-40% EA in PE) to give N3T12-017-01-3 (10.0 g, 16.290 mmol, 60.49% yield) as a white solid. LCMS: m/z 614.4 [(M+H)⁺].

N3T12-022-01-1: To a solution of N3T12-017-01-3 (10.0 g, 16.290 mmol) in pyridine (150 mL) is added methanesulfonyl chloride (3.8 g, 0.033 mol). The resulting mixture is stirred for overnight at rt. Then, water (15 mL) is added to quench the reaction and evaporated to dryness under reduced pressure. The residue is purified by column chromatography over silica gel (eluting with 0%-30% EA in PE) to give N3T12-022-01-1 (9.4 g, 14.828 mmol, 91.13%) as a white solid. LCMS: m/z 692.4 [(M+H)⁺].

N3T12-022-01-3: To a solution of NaH (0.85 g, 0.021 mol) in N,N-dimethylmethanamide (120 mL) is added hexadecane-1-thiol (5.49 g, 0.021 mol) at 0° C. under nitrogen atmosphere. The mixture is stirred at 0° C. for 30 min. Then, N3T12-022-01-1 (4.9 g, 0.007 mol) is added and stirred at rt for 3 h. After the reaction is completed, water is added to quench the reaction. Then, the mixture is poured into brine (200 mL). The solution is extracted with DCM (200*3) and the organic phase is dried with anhydrous sodium sulfate, filtered and evaporated to dryness under reduced pressure. The residue is purified by column chromatography (eluting with 0%-5% MeOH in DCM) to give N3T12-022-01-3 (1.7 g, 2.778 mmol, 39.24% yield) as a white solid. LCMS: m/z 612.5 [(M+H)⁺].

N3T12-022-01-4: To a solution of N3T12-022-01-3 (1.7 g, 0.003 mol) in pyridine (20 mL) is added DMTrCl (1.9 g, 0.006 mol) in 10 mL THF. The resulting mixture is stirred for overnight at rt. Then, water (10 mL) is added to quench the reaction and evaporated to dryness under reduced pressure. The residue is purified by column chromatography over silica gel (eluting with 0%-50% EA in PE) to give N3T12-022-01-4 (1.1 g, 1.203 mmol, 43.31% yield) as a yellow solid. LCMS: m/z 914.7 [(M+H)⁺].

N3T12-022-01: To a solution of N3T12-022-01-4 (1.1 g, 0.001 mol) and di(prop-2-yl) ammonium 1,2,3,4-tetraazol-1-ide (0.31 g, 0.002 mol) in DMF (4 mL) is added 3-{[2,6-dimethyl-3,5-di(prop-2-yl)-3,5-diaza-4-phosphahept-4-yl]oxy}propanenitrile (0.73 g, 0.002 mol) in ACN (2 mL). The mixture is stirred at rt for 16 hr in the sealed tube. After the reaction is completed, the mixture is poured into a saturated sodium bicarbonate solution (100 mL). Then, the solution is extracted with DCM (100*3). The organic phase is dried with anhydrous sodium sulfate, filtered and evaporated to dryness under reduced pressure. The residue is purified by reversed phase column (100% ACN) to give N3T12-022-01 (1.3 g, 1.167 mmol, 96.95% yield) as a white solid. LCMS (TM hydrolysate): m/z 1031.9 [(M+H)⁺]. ¹H NMR (400 MHz, DMSO-d₆) δ 11.22 (d, J=6.6 Hz, 1H), 8.65 (d, J=10.0 Hz, 1H), 8.45 (d, J=16.2 Hz, 1H), 8.05 (dd, J=7.4, 2.2 Hz, 2H), 7.68-7.62 (m, 1H), 7.58-7.52 (m, 2H), 7.40 (d, J=7.2 Hz, 2H), 7.31-7.16 (m, 7H), 6.83 (dd, J=8.6, 6.8 Hz, 4H), 6.15 (dd, J=8.6, 5.0 Hz, 1H), 5.28-5.09 (m, 1H), 4.68-4.61 (m, 1H), 3.86-3.77 (m, 1H), 3.74-3.63 (m, 7H), 3.51-3.69 (m, 2H), 3.39-3.48 (m, 2H), 2.77 (t, J=6.0 Hz, 1H), 2.35-2.54 (m, 3H), 1.32-1.44 (m, 2H), 1.30-1.15 (m, 27H), 1.13 (d, J=6.6 Hz, 3H), 1.01-1.10 (m, 6H), 0.84 (t, J=6.8 Hz, 3H), 0.74 (d, J=6.6 Hz, 3H). 31P NMR (400 MHz, DMSO-d₆) δ 150.67 (s), 149.84 (s).

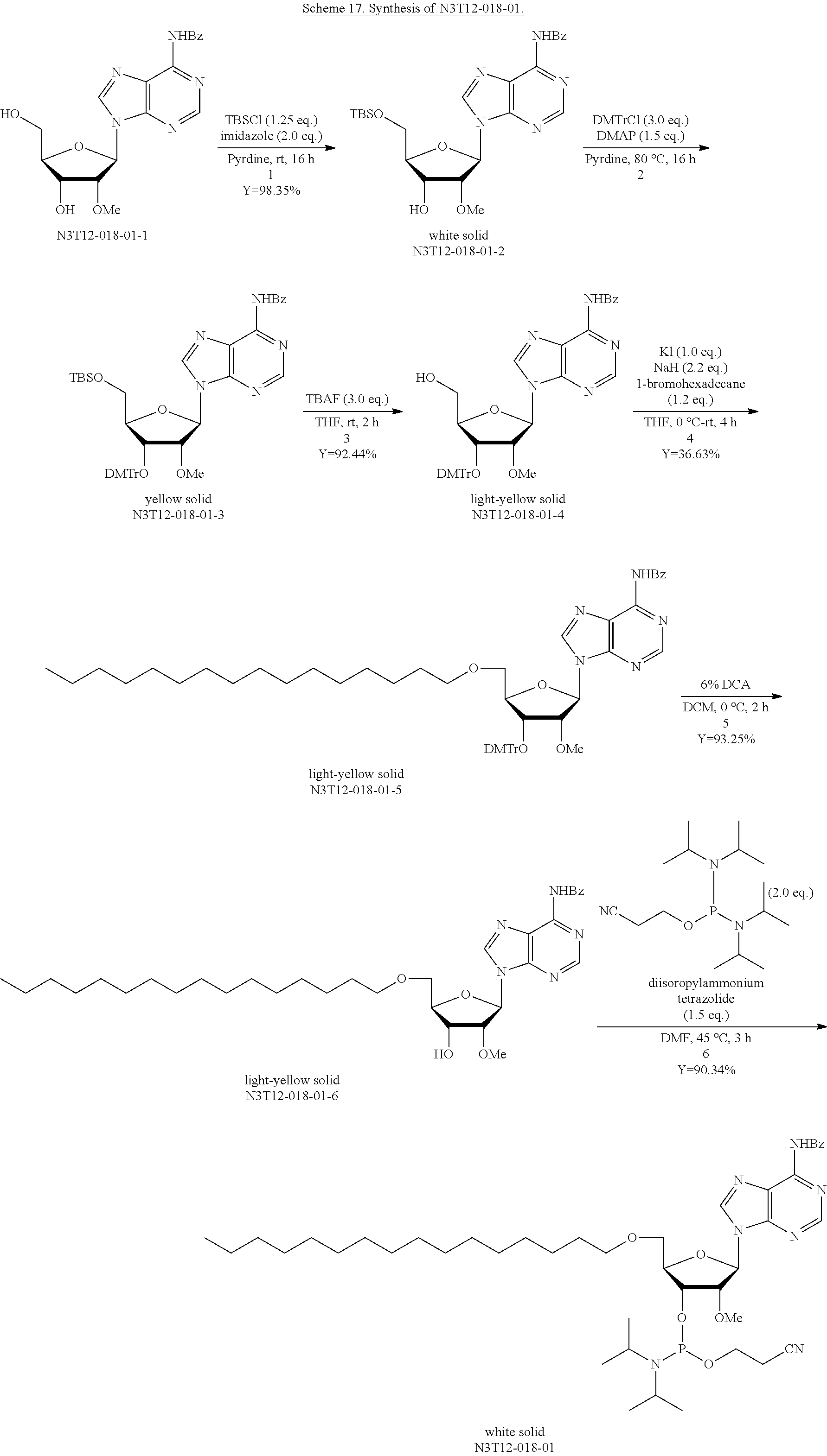

N3T12-018-01-2: To a solution of N3T12-018-01-1 (4 g, 10.38 mmol) in Pyridine (50 mL) is added imidazole (1.41 g, 20.76 mmol) and chlorodimethyl(2-methylprop-2-yl) silane (1.96 g, 12.97 mmol). The reaction is stirred for 16 h at rt under N₂ atmosphere. The reaction is quenched by water (50 mL) and extracted with DCM (3*50 mL), the combined organic layer is dried over Na₂SO₄ and the solvent is removed in vacuo. The residue is purified by flash column chromatography (eluting with 0%-5% MeOH in DCM) to afford N3T12-018-01-2 (5.1 g, 10.21 mmol, 98.35% yield) as a white solid. LCMS: m/z 500.4 [(M+H)⁺]. ¹H NMR (400 MHz, DMSO-d₆) δ 11.22 (s, 1H), 8.76 (s, 1H), 8.62 (s, 1H), 8.08-8.01 (m, 2H), 7.69-7.61 (m, 1H), 7.57-7.54 (m, 2H), 6.18 (d, J=4.8 Hz, 1H), 5.37 (s, 1H), 4.47-4.34 (m, 2H), 4.02 (q, J=4.0 Hz, 1H), 3.90 (dd, J=11.4, 3.8 Hz, 1H), 3.79 (dd, J=11.4, 4.2 Hz, 1H), 3.39 (s, 3H), 0.87 (s, 9H), 0.05 (s, 6H).

N3T12-018-01-3: To a solution of N3T12-018-01-2 (5.1 g, 10.21 mmol) in pyrdine (60 mL) is added 4-(dimethylamino)pyridine (1.87 g, 15.31 mmol) and 1-[chloro(4-methoxyphenyl)phenylmethyl]-4-methoxybenzene (10.38 g, 30.62 mmol). The reaction is stirred for 16 hr at 80° C. under N₂ atmosphere. The reaction is quenched by water (50 mL) and extracted with DCM (3*50 mL). The combined organic layer is dried over Na₂SO₄ and the solvent is removed in vacuo to afford N3T12-018-01-3 (8.2 g, crude) as a yellow solid. The crude product is used for the next step without further purification. LCMS: m/z 802.6 [(M+H)⁺].

N3T12-018-01-4: To a solution of N3T12-018-01-3 (8.2 g, 10.22 mmol) in tetrahydrofuran (30 mL) is added TBAF (1M, 30.67 mL). The reaction is stirred for 2 h at rt. The solvent is extracted with H₂O/DCM (3*50 mL) and the combined organic layer is dried Na₂SO₄. The solvent is removed in vacuo and the residue is purified by flash column chromatography (eluting with 0%-5% MeOH in DCM) to afford N3T12-018-01-4 (6.5 g, 9.45 mmol, 92.44% yield) as a light-yellow solid. LCMS: m/z 688.4 [(M+H)⁺]. ¹H NMR (400 MHz, DMSO-d₆) δ 11.22 (s, 1H), 8.75 (s, 1H), 8.69 (s, 1H), 8.07-8.02 (m, 2H), 7.67-7.63 (m, 1H), 7.58-7.50 (m, 4H), 7.39-7.31 (m, 6H), 7.26-7.22 (m, 1H), 6.92-6.86 (m, 4H), 6.33 (d, J=5.4 Hz, 1H), 5.06 (t, J=5.2 Hz, 1H), 4.29-4.21 (m, 1H), 3.82 (t, J=5.0 Hz, 1H), 3.73 (d, J=2.8 Hz, 6H), 3.41-3.36 (m, 1H), 3.25-3.18 (m, 4H).

N3T12-018-01-5: To a solution of N3T12-018-01-4 (6.5 g, 9.45 mmol) in N,N-dimethylmethanamide (50 mL) is added potassium iodide (1.57 g, 9.45 mmol) and NaH (0.83 g, 20.79 mmol). The reaction is stirred for 0.5 h at 0° C. under N₂ atmosphere. Then 1-bromohexadecane (3.46 g, 11.34 mmol) is added and the reaction is stirred for 4 h at rt under N₂ atmosphere. The reaction is quenched by NH₄Cl (aq, 50 mL) and extracted with DCM (3*50 mL). The combined organic layer is dried over Na₂SO₄ and the solvent is removed in vacuo. The residue is purified by flash column chromatography (eluting with 0%-5% MeOH in DCM) to afford N3T12-018-01-5 (3.16 g, 3.46 mmol, 36.65% yield) as a light-yellow solid. ¹H NMR (400 MHz, DMSO-d₆) δ 11.19 (s, 1H), 8.71 (s, 1H), 8.54 (s, 1H), 8.09-8.02 (m, 2H), 7.68-7.61 (m, 1H), 7.57-7.53 (m, 2H), 7.49-7.45 (m, 2H), 7.32-7.28 (m, 6H), 7.23-7.19 (t, J=7.3 Hz, 1H), 6.86-6.81 (m, 4H), 6.23 (d, J=3.4 Hz, 1H), 4.32-4.26 (m, 1H), 3.96-6.93 (m, 1H), 3.69 (d, J=5.2 Hz, 6H), 3.51-3.45 (m, 1H), 3.43-3.39 (m, 1H), 3.31-3.29 (m, 1H), 3.26 (s, 3H), 3.21-3.16 (m, 2H), 1.37-1.30 (m, 2H), 1.24-1.11 (m, 26H), 0.84 (t, J=6.8 Hz, 3H).

N3T12-018-01-6: To a solution of N3T12-018-01-5 (3.16 g, 3.46 mmol) in DCM (40 mL) is added DCA (2.4 mL). The reaction is stirred for 2 h at 0° C. The reaction is quenched by MeOH (20 mL) and neutralized by NaHCO₃(aq, 50 mL). The mixture is extracted with DCM (3*50 mL) and the combined organic layer is dried over Na₂SO₄. The solvent is removed in vacuo and the residue is purified by flash column chromatography (eluting with 0%-5% MeOH in DCM) to afford N3T12-018-01-6 (1.97 g, 3.23 mmol, 93.25% yield) as a light-yellow solid. ¹H NMR (400 MHz, DMSO-d₆) δ 11.23 (s, 1H), 8.77 (s, 1H), 8.66 (s, 1H), 8.08-8.02 (m, 2H), 7.65 (t, J=7.4 Hz, 1H), 7.55 (t, J=7.6 Hz, 2H), 6.18 (d, J=4.8 Hz, 1H), 5.41 (d, J=5.6 Hz, 1H), 4.42-4.36 (m, 2H), 4.08 (q, J=4.0 Hz, 1H), 3.67 (dd, J=10.8, 3.6 Hz, 1H), 3.57 (dd, J=10.8, 4.6 Hz, 1H), 3.47-3.41 (m, 2H), 3.39 (s, 3H), 1.55-1.47 (m, 2H), 1.27-1.19 (m, 26H), 0.85 (t, J=6.8 Hz, 3H).

N3T12-018-01: To a solution of N3T12-018-01-5 (0.99 g, 3.28 mmol) Diisoropyl Ammonium Tetrazolide (211 mg, 1.23 mmol) in N,N-dimethylmethanamide (20 mL) is added 3-{[2,6-dimethyl-3,5-di(prop-2-yl)-3,5-diaza-4-phosphahept-4-yl]oxy}propanenitrile (0.99 g, 3.28 mmol). The reaction is stirred for 3 h at 45° C. under N₂ atmosphere. The mixture is extracted with NaHCO₃(aq)/DCM (3*50 mL) and the combined organic layer is dried over Na₂SO₄. The solvent is removed in vacuo and the residue is purified by prep.HPLC (eluting with 100% ACN) to afford N3T12-018-01 (1.2 g, 1.48 mmol, 90.34% yield) as a white solid. LCMS: m/z 727.5 [(M−DIPA+OH+H)⁺]. ¹H NMR (400 MHz, DMSO-d6) δ 11.24 (s, 1H), 8.76 (s, 1H), 8.67 (d, J=5.8 Hz, 1H), 8.05 (d, J=7.2 Hz, 2H), 7.65 (t, J=7.4 Hz, 1H), 7.55 (t, J=7.6 Hz, 2H), 6.20-6.16 (m, 1H), 4.74-4.58 (m, 2H), 4.31-4.19 (m, 1H), 3.88-3.54 (m, 6H), 3.48-3.36 (m, 5H), 2.84-2.80 (m, 2H), 1.54-1.46 (m, 2H), 1.27-1.15 (m, 38H), 0.85 (t, J=6.8 Hz, 3H). ³¹P NMR (162 MHz, DMSO-d6) δ 149.90, 149.16.

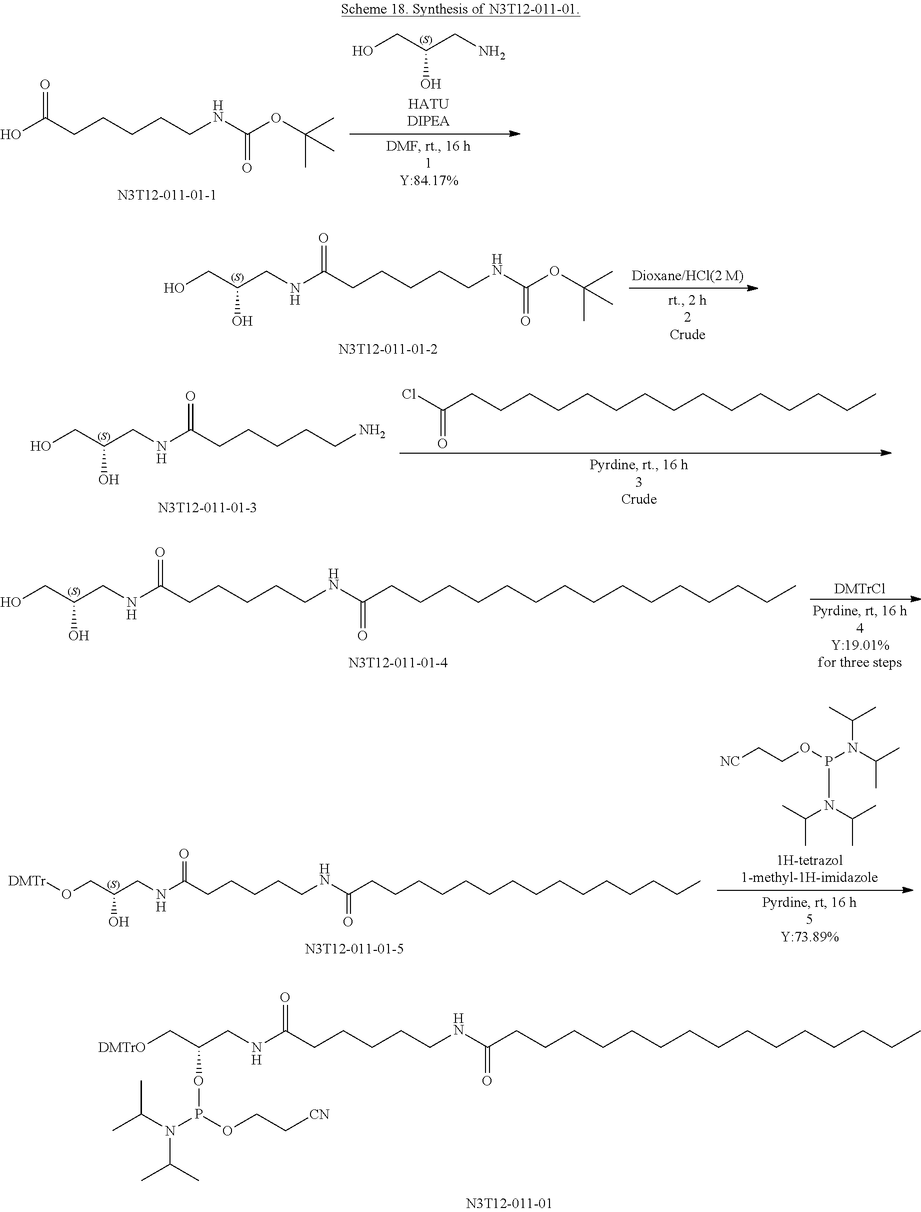

N3T12-011-01-2: To a solution of N3T12-011-01-1 (6.5 g, 0.028 mol) in N,N-dimethylmethanamide (150 mL) is added HATU (16 g, 0.042 mol) and DIPEA (10.9 g, 0.084 mol). The mixture is stirred at room temperature (rt) for 30 minutes. Then, (S)-3-aminopropane-1,2-diol (3.0 g, 0.033 mol) is added, the mixture is stirred at rt for another 16 hours. After the reaction is complete, the solvent is directly concentrated under reduced pressure (by oil pump). The resultant residue is purified by flash silica gel column chromatography (eluting with 0%-20% MeOH in DCM) to afford N3T12-011-01-2 (7.2 g, 23.654 mmol, 84.17% yield) as a brown oil. LCMS: m/z 305.1 [(M+H)⁺].

N3T12-011-01-3: N3T12-011-01-2 (7.2 g, 23.654 mmol) is added to a solution of dioxane/HCl (2 M, 40 mL). The mixture is stirred at rt for 2 hours. After the reaction is complete, the solvent is concentrated under reduced pressure to afford N3T12-011-01-3 (6.6 g, crude) as a yellow oil and the resulting crude is used for the next step directly without further purification. LCMS: m/z 205.1 [(M+H)⁺].

N3T12-011-01-4: To a solution of N3T12-011-01-3 (6.6 g, 0.032 mol) in pyrdine (50 mL) is added hexadecanoyl chloride (10.6 g, 0.039 mol). The mixture is stirred at room temperature (rt) for overnight. After the reaction is complete, the solvent is concentrated under reduced pressure to afford N3T12-011-01-4 (15.9 g, crude) as a yellow oil and the resulting crude is used for the next step directly without further purification. LCMS: m/z 443.5 [(M+H)⁺].

N3T12-011-01-5: To a solution of N3T12-011-01-4 (5.0 g, 0.011 mol) in pyrdine (20 mL) is added DMTrCl (4.5 g, 0.013 mol). The mixture is stirred at rt for overnight. After the reaction is complete, water (50 mL) is added to quench the reaction. Then, the combined organic layer is washed with water (150 mL) and brine, dried over Na₂SO₄, and concentrated to give a residue which is purified by silica gel column chromatography (eluting with 0%-5% MeOH in DCM) to afford N3T12-011-01-5 (1.6 g, 2.147 mmol, 19.01% yield) as a yellow solid. LCMS: m/z 767.6 [(M+Na)⁺].

N3T12-011-01: To a solution of N3T12-011-01-5 (1.6 g, 2.147 mmol) in DMF (6 mL) is added 3-{[2,6-dimethyl-3,5-di(prop-2-yl)-3,5-diaza-4-phosphahept-4-yl]oxy}propanenitrile (971 mg, 3.221 mmol), 1-methylimidazole (265 mg, 3.221 mol) and 3H-1,2,3,4-tetraazole (181 mg, 2.577 mmol) successively. The mixture is stirred at rt for 16 hours in a sealed tube. After the reaction is complete, the mixture is poured into a saturated sodium bicarbonate solution (100 mL). Then, the solution is extracted with DCM (100*3). The organic phase is dried with anhydrous sodium sulfate, filtered and evaporated to dryness under reduced pressure. The residue is purified by silica column (PE/EA/0.1% Et₃N) and reverse phase column (99.9% ACN/0.1% Et₃N) to give N3T12-011-01 (1.5 g, 1.587 mmol, 73.89% yield) as a colorless oil. LCMS (TM hydrolysate): m/z 884.6 [(M+Na)⁺]. ¹H NMR (400 MHz, DMSO) δ 7.70 (t, J=5.4 Hz, 1H), 7.63 (dt, J=23.8, 5.8 Hz, 1H), 7.43-7.36 (m, 2H), 7.33-7.18 (m, 7H), 6.91-6.82 (m, 4H), 4.00 (dd, J=10.4, 5.8 Hz, 1H), 3.83-3.70 (m, 7H), 3.70-3.47 (m, 3H), 3.31 (s, 1H), 3.24-3.06 (m, 2H), 3.03-2.88 (m, 3H), 2.77 (dd, J=9.0, 3.2 Hz, 1H), 2.63 (td, J=5.8, 1.6 Hz, 1H), 2.02 (t, J=7.4 Hz, 2H), 2.00-1.89 (m, 2H), 1.51-1.25 (m, 8H), 1.24-1.10 (m, 33H), 1.01 (d, J=6.6 Hz, 3H), 0.85 (t, J=6.8 Hz, 3H). ³¹P NMR (400 MHz, DMSO-d₆) δ 148.41 (s), 147.91 (s).

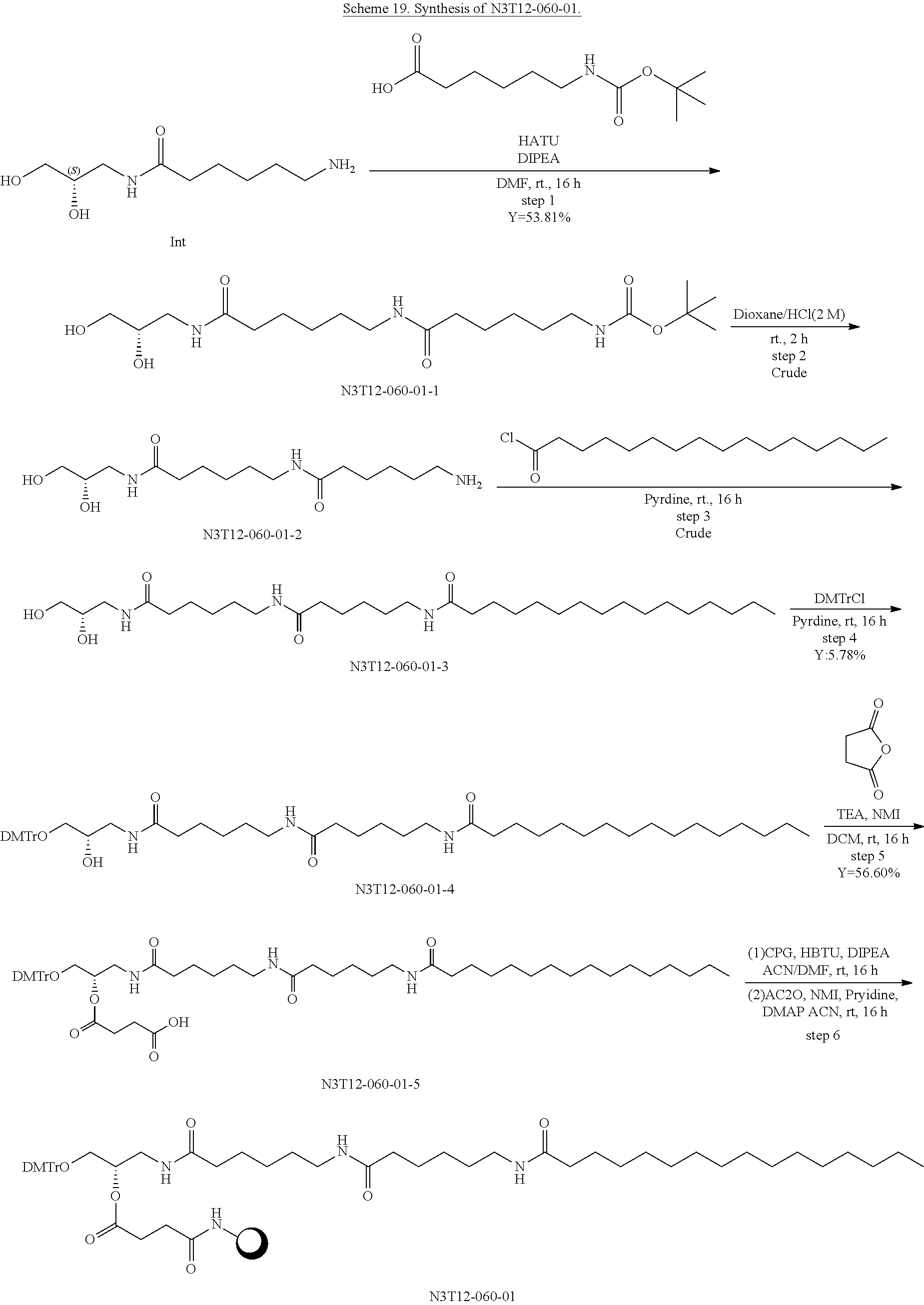

N3T12-060-01-1: To a solution of 6-((tert-butoxycarbonyl)amino) hexanoic acid (1.0 g, 4.406 mmol) in dry-DMF (20 mL) is added HATU (2.4 g, 6.364 mmol) and DIPEA (6.3 g, 48.955 mmol). The mixture is stirred at rt for 30 minutes. Then, (S)-6-amino-N-(2,3-dihydroxypropyl) hexanamide (1.0 g, 4.895 mmol) is added, the mixture is stirred at rt for another 16 hours. After the reaction is complete, the solvent is concentrated under reduced pressure. The resultant residue is purified by flash silica gel column chromatography (0%-20% MeOH in DCM) to afford N3T12-060-01-1 (1.1 g, 2.634 mmol, 53.81%) as a brown oil. LCMS: m/z 418.5 [(M+H)⁺].

N3T12-060-01-2: N3T12-060-01-1 (1.1 g, 2.634 mmol) is added to a solution of dioxane/HCl (2 M, 10 mL). The mixture is stirred at rt for 2 hours. After the reaction is complete, the solvent is concentrated under reduced pressure. The N3T12-060-01-2 (1.0 g, crude) is used for the next step without further purification.

N3T12-060-01-3: To a solution of N3T12-060-01-2 (900 mg, 2.835 mmol) in dry pyridine (10 mL) is added hexadecanoyl chloride (779 mg, 2.835 mmol). The mixture is stirred at rt for 16 hours. After the reaction is complete, N3T12-060-01-3 (1.6 g, crude) is used for the next step without further purification. LCMS: m/z 556.6 [(M+H)⁺].

N3T12-060-01-4: N3T12-060-01-3 (1.4 g, 2.519 mmol) in dry pyridine (25 mL) is added DMTrCl (1.3 g, 3.778 mmol). The mixture is stirred at rt for 16 hours. After the reaction is complete, the solution is diluted with ethyl acetate (EA) (150 mL). The organic layer is washed with water and brine, dried over Na₂SO₄, and concentrated under reduced pressure. The resultant residue is purified by flash silica gel column chromatography (0%-4% MeOH in DCM) to afford N3T12-060-01-4 (125 mg, 0.146 mmol, 5.78%) as a white solid. LCMS: m/z 880.9 [(M+Na)⁺].

N3T12-060-01-5: To a solution of N3T12-060-01-4 (125 mg, 145.650μ mol) in dry DCM (5 mL) is added tetrahydrofuran-2,5-dione (44 mg, 436.951μ mol), triethylamine (45 mg, 436.951μ mol) and 3-methylimidazole (13 mg, 160.215μ mol). The mixture is stirred at rt for 16 hours. After the reaction is complete, the solution is diluted with EA (150 mL). The organic layer is washed with water and brine, dried over Na₂SO₄, and concentrated under reduced pressure. The resultant residue is purified by flash silica gel column chromatography (0%-7% MeOH in DCM) to afford N3T12-060-01-5 (79 mg, 0.082 mmol, 56.60%) as a white solid. LCMS: m/z 980.8 [(M+Na)⁺].

N3T12-060-01: To the solution of N3T12-060-01-5 (79 mg, 79.308 μmol) in the mixture solution of ACN (9 mL) and N,N-dimethylmethanamide (3 mL) are added DIPEA (31 mg, 237.924 umol) and HBTU (68 mg, 178.443 μmol) in a centrifugal tube. CPG (350 mg, 63 μmol) is added to the mixture solution. The tube is placed on a wrist-action shaker and shaken (220 r/min) at 25° C. for 16 hours. The reaction solution is filtered, and the filter cake is washed with dichloromethane (DCM) (30 mL×3), acetonitrile (CAN) (30 mL×3) and n-hexane (30 mL×3), respectively. After drying in vacuo, the filter cake yields the crude N3T12-060-01 (320 mg) as a white solid. The loading value of CPG is measured (CPG loading value=77.21 μmol/g). To the solution of the crude N3T12-060-01 (320 mg, 33 umol) in the mixture solution of ACN (10 mL) are added 4-(dimethylamino)pyridine (1 mg, 9.900 umol), Pyridine (78. mg, 990.000 μmol), NMI (1 mg, 9.900 μmol), and Ac2O (34 mg, 330.000 μmol) in a centrifugal tube. The tube is placed on a wrist-action shaker and shaken (220 r/min) at 25° C. for 16 hours. The reaction solution is filtered, and the filter cake is washed with ACN (30 mL×3). After drying the filter cake in vacuo to give N3T12-060-01-2 (287.80 mg) as a white solid, the loading value of CPG is measured (CPG loading value=70.93 umol/g).

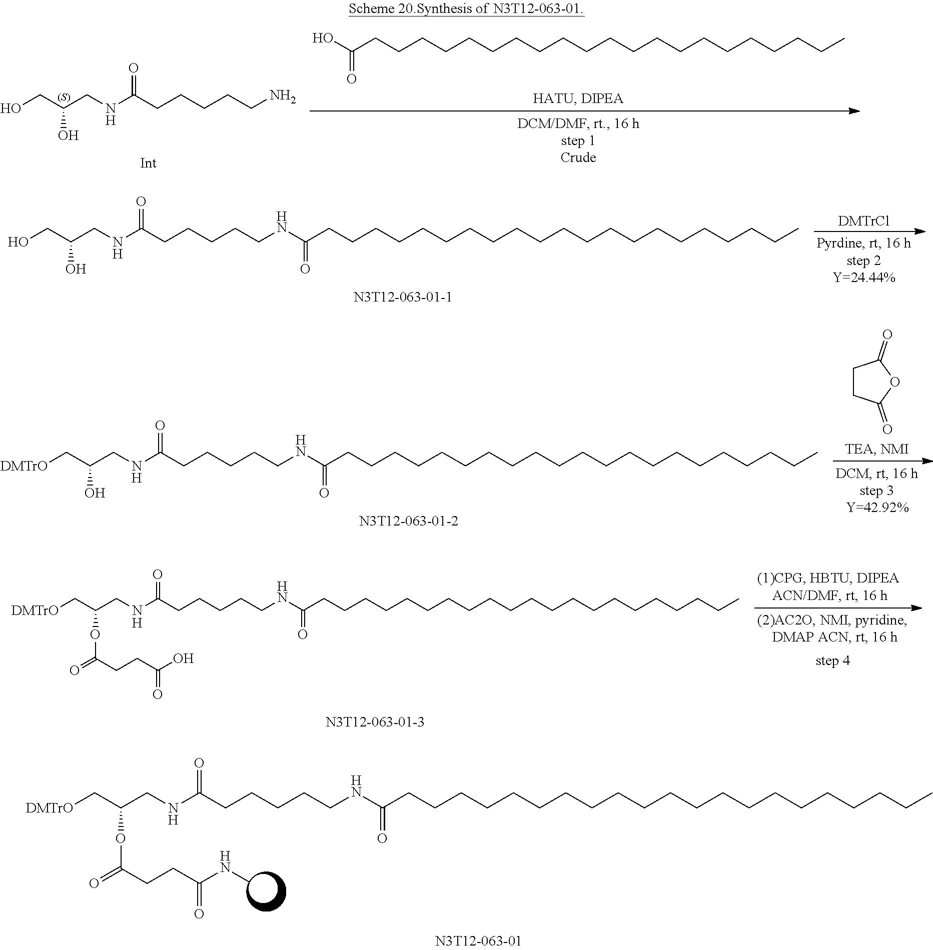

N3T12-063-01-1: To a solution of docosanoic acid (996 mg, 2.923 mmol) in dry DCM (20 mL) and dry DMF (20 mL) is added HATU (1.2 g, 3.166 mmol) and DIPEA (4.7 g, 36.535 mmol). The mixture is stirred at rt for 2 hours. Then, (S)-6-amino-N-(2,3-dihydroxypropyl) hexanamide (500 mg, 2.436 mmol) is added. The mixture is stirred at rt for another 16 hours. After the reaction is complete, the solvent is concentrated under reduced pressure to afford N3T12-063-01-1 (1.3 g, crude) as brown oil. The crude is used for the next step without further purification.

N3T12-063-01-2: To N3T12-063-01-1 (1.3 g, 2.467 mmol) in dry pyridine (15 mL) is added DMTrCl (1.3 g, 3.701 mmol) in 8 mL THF. The mixture is stirred at rt for 16 hours. After the reaction is complete, the solution is diluted with EA (150 mL). The organic layer is washed with water and brine, dried over Na₂SO₄, and concentrated under reduced pressure. The resultant residue is purified by flash silica gel column chromatography (0%-4% MeOH in DCM) to afford N3T12-063-01-2 (500 mg, 0.603 mmol, 24.44%) as a light yellow solid. LCMS: m/z 851.7 [(M+Na)⁺]¹H NMR (400 MHz, DMSO-d₆) δ 7.70 (t, J=5.6 Hz, 1H), 7.66 (t, J=5.6 Hz, 1H), 7.40 (d, J=7.6 Hz, 2H), 7.33-7.17 (m, 7H), 6.90-6.84 (m, 4H), 4.97 (d, J=5.0 Hz, 1H), 3.76-3.65 (m, 7H), 3.31-3.22 (m, 1H), 3.02-2.81 (m, 5H), 2.01 (dd, J=14.0, 6.8 Hz, 4H), 1.51-1.30 (m, 6H), 1.30-1.13 (d, J=20.5 Hz, 38H), 0.85 (t, J=6.6 Hz, 3H).

N3T12-063-01-3: To a solution of N3T12-063-01-2 (420 mg, 0.507 mmol) in dry DCM (10 mL) are added tetrahydrofuran-2,5-dione (152 mg, 1.520 mmol), triethylamine (154 mg, 1.520 mmol) and 3-methylimidazole (46 mg, 0.557 mmol). The mixture is stirred at rt for 16 hours. After the reaction is complete, the resultant residue is purified by flash silica gel column chromatography (0%-7% MeOH in DCM) and reverse phase column chromatography (100% ACN) to afford N3T12-063-01-3 (202 mg, 0.217 mmol, 42.92%) as a white solid. LCMS: m/z 952.4 [(M+Na)⁺] ¹H NMR (400 MHz, DMSO-d₆) δ 11.42 (s, 1H), 7.79 (t, J=5.8 Hz, 1H), 7.71 (t, J=5.6 Hz, 1H), 7.38-7.27 (m, 4H), 7.22 (d, J=8.8 Hz, 5H), 6.88 (d, J=8.8 Hz, 4H), 5.04-4.97 (m, 1H), 3.73 (s, 6H), 3.38-3.29 (m, 1H), 3.16-3.08 (m, 1H), 3.07-3.01 (m, 2H), 3.00-2.94 (m, 2H), 2.60-2.53 (m, 2H), 2.49-2.46 (m, 2H), 1.99 (dt, J=21.2, 7.4 Hz, 4H), 1.50-1.42 (m, 2H), 1.41-1.28 (m, 4H), 1.29-1.12 (m, 38H), 0.85 (t, J=6.6 Hz, 3H).

N3T12-063-01: To the solution of N3T12-063-01-3 (51 mg, 54.881 umol) in the mixture solution of ACN (9 mL) and N,N-dimethylmethanamide (3 mL) are added DIPEA (14 mg, 109.761 umol) and HBTU (31 mg, 82.321 μmol) in a centrifugal tube. CPG (250 mg, 36 umol) is added to the mixture solution. The tube is placed on a wrist-action shaker and shaken (220 r/min) at 25° C. for 16 hours (h). The reaction solution is filtered, and the filter cake is washed with DCM (30 mL×3), ACN (30 mL×3) and n-hexane (30 mL×3), respectively. After drying the filter cake in vacuo to give the crude N3T12-063-01 (240 mg) as a white solid, the loading value of CPG is measured (CPG loading value=80.31 μmol/g). To the solution of the crude N3T12-063-01 (240 mg, 99.69 μmol) in the mixture solution of ACN (10 mL) are added 4-(dimethylamino)pyridine (4 mg, 29.907 umol), Pyridine (237 mg, 2990.700 umol), NMI (3 mg, 29.907 umol) and Ac2O (102 mg, 996.900 μmol) in a centrifugal tube. The tube is placed on a wrist-action shaker and shaken (220 r/min) at 25° C. for 16 hours. The reaction solution is filtered, and the filter cake is washed with ACN (30 mL×3). After drying the filter cake in vacuo to give N3T12-063-01 (219.77 mg) as a white solid, the loading value of CPG is measured (CPG loading value=70.69 μmol/g).

### Example 7. Dose Screens of VEGFA siRNA Duplexes in HuH7 Cells

The siRNAs are transfected into Human Hepatocyte-derived cell line HuH7 with RNAiMAX (Invitrogen, 13778075) in a 96-well plate. All the siRNAs are tested at 10 nM and 1 nM (Tables 9-10).

Twenty-four hours after transfection, the culture medium is discarded and cells are collected for RNA extraction. Total RNA is extracted by RNeasy® 96 Kit (QIAGEN-74182) according to the kit instructions, and reverse transcribed to cDNA by FastKing RT Kit (with gDNase) (Tiangen-KR116-02).

The cDNA of target gene is quantified by qPCR with specific primer and probe sets. β-actin cDNA is measured as housekeeping gene in parallel. 8 μL PCR mixture and 2 μL sample cDNA are added to 384-well PCR plates. The PCR is performed with 95° C. for 10 minutes, then cycling at 95° C. 15 seconds and 60° C. 1 minute for 40 cycles.

TABLE 9
Modified Human VEGFA siRNA Duplexes Conjugated with Lipophilic Moieties

| | SEQ | | SEQ | |
|---|---|---|---|---|
| Duplex | ID NO | sense strand (5′ to 3′) | ID NO | antisense strand (5′ to 3′) |
| N3T070127- | 201 | UmsGmsGmUmUfAmAfCfAfUmGmUmCmG | 202 | VPUms UfsAmGmUmCfGmAfCfAmUm |
| 01 (negative | | mAmCmUmAmsAms[C5Cholesterol] | | GmGmAfAmAfCmCmAmsUmsUm |
| control) | | | | |
| N3T070128- | 203 | AmsGms AmGmAfAmAfAfGfAmGmAmAmA | 204 | VPUmsAfsCmAmCmUfUmUfCfUmCm |
| 01 (positive | | mGmUmGmUmsUms[C5Cholesterol] | | UmUmUfUmCfUmCmUmsUmsUm |
| control) | | | | |
| N3T070129- | 205 | AmsGms AmGmAfAmAfAfGfAmGmAmAmA | 206 | VPUmsAfsCmAmCmUfUmUfCfUmCm |
| 01 | | mGmUmGmUmsUms[N3T12-010-01] | | UmUmUfUmCfUmCmUmsUmsUm |
| N3T070130- | 207 | AmsGmsAmGmAfAmAfAfGfAmGmAmAmA | 208 | VPUmsAfsCmAmCmUfUmUfCfUmCm |
| 01 | | mGmUmGmUmsUms[N3T12-012-01] | | UmUmUfUmCfUmCmUmsUmsUm |
| N3T070131- | 209 | AmsGmsAmGmAfAmAfAfGfAmGmAmAmA | 210 | VPUmsAfsCmAmCmUfUmUfCfUmCm |
| 01 | | mGmUmGmUmsUms[N3T12-023-01] | | UmUmUfUmCfUmCmUmsUmsUm |
| N3T070132- | 211 | [N3T12-019-01] | 212 | VPUmsAfsCmAmCmUfUmUfCfUmCm |
| 01 | | sAmsGmAmGmAfAmAfAfGfAmGmAmA | | UmUmUfUmCfUmCmUmsUmsUm |
| | | mAmGmUmGmsUmsUm | | |
| N3T070133- | 213 | [N3T12-019-01] | 214 | VPUmsAfsCmAmCmUfUmUfCfUmCm |
| 01 | | sAmsGmAmGmAfAmAfAfGfAmGmAmA | | UmUmUfUmCfUmCmUmsUmsUm |
| | | mAmGmUmGmUmsUms[N3T12-023-01] | | |
| N3T070134- | 215 | AmsGmsAmGmAf[N3T12-022- | 216 | VPUmsAfsCmAmCmUfUmUfCfUmCm |
| 01 | | 01]AfAfGfAmGmAmAmAmGmUmGmsUms | | UmUmUfUmCfUmCmUmsUmsUm |
| | | Um | | |
| N3T070135- | 217 | [N3T12-018-01] | 218 | VPUmsAfsCmAmCmUfUmUfCfUmCm |
| 01 | | sAmsGmAmGmAfAmAfAfGfAmGmAmA | | UmUmUfUmCfUmCmUmsUmsUm |
| | | mAmGmUmGmsUmsUm | | |
| N3T070136- | 219 | AmsGmsAmGmAf[Ahd]AfAfGfAmGmAmA | 220 | VPUmsAfsCmAmCmUfUmUfCfUmCm |
| 01 | | mAmGmUmGmsUmsUm | | UmUmUfUmCfUmCmUmsUmsUm |
| N3T070137- | 402 | AmsGmsAmGmAfAmAfAfGfAmGmAmAmA | 403 | VPUmsAfsCmAmCmUfUmUfCfUmCm |
| 01 | | mGmUmGmUmsUms[N3T12-024-01] | | UmUmUfUmCfUmCmUmsUmsUm |

Am, Cm, Gm, Um indicate 2′-O-methyl (2′-OMe) sugar modifications, respectively, to adenosine, cytidine,
guanosine and uridine; Af, Cf, Gf, Uf indicate 2′-deoxy-2′-fluoro (2′-F) sugar modifications, respectively,
to adenosine, cytidine, guanosine and uridine; s indicates phosphorothioate (PS) linkage; VPUm indicates
vinyl phosphonate.

Structures of lipid modifications listed in Table 9 include:

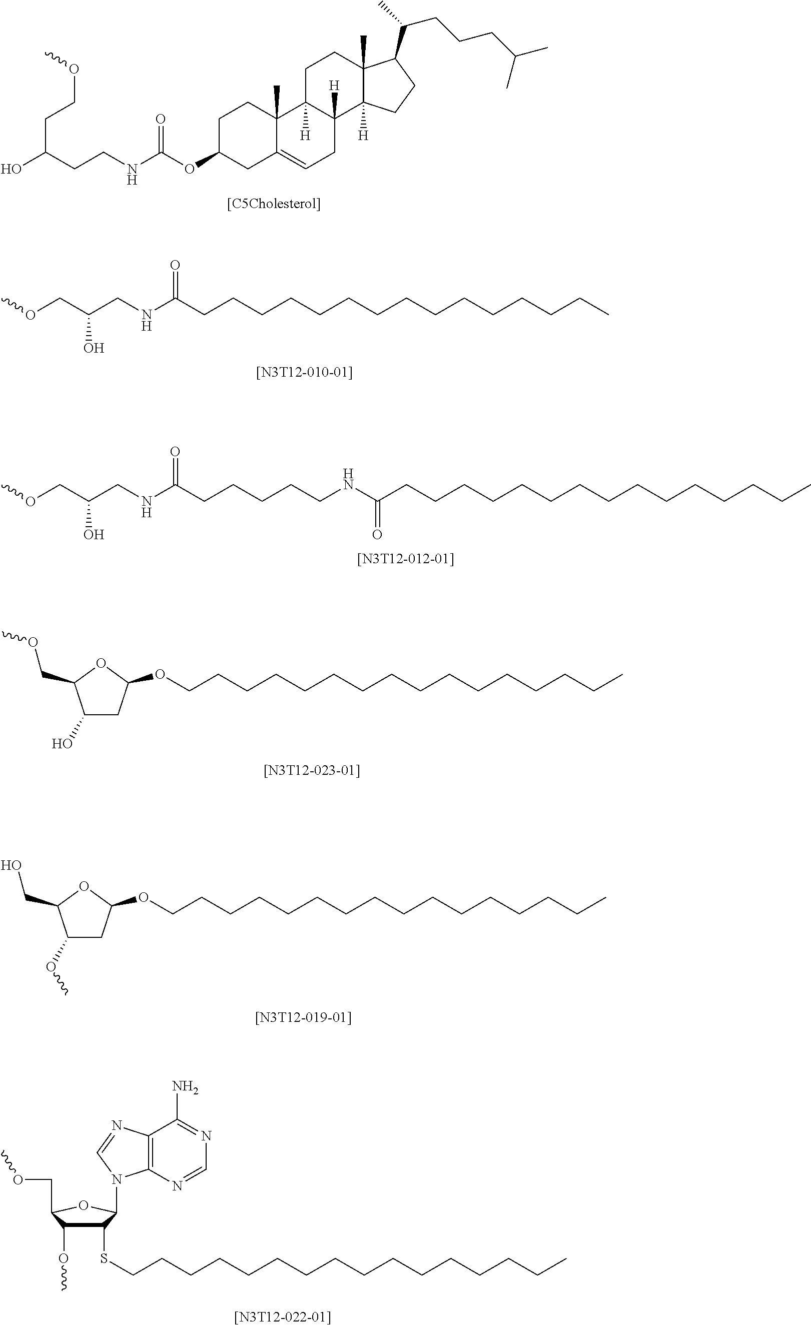

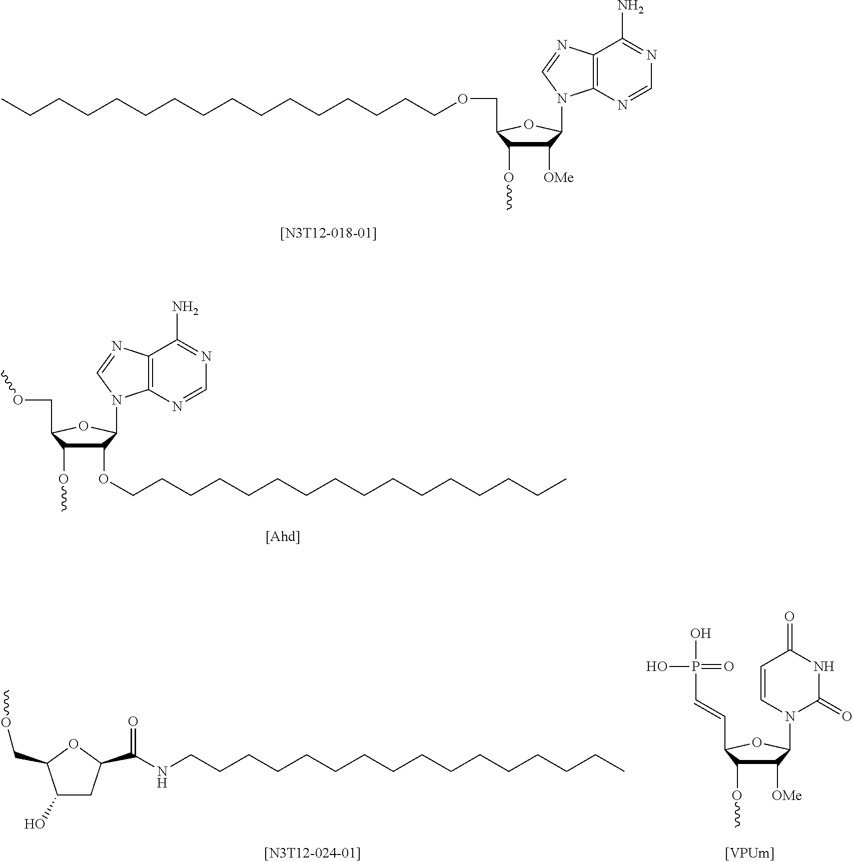

TABLE 10
Dose Screens of VEGFA siRNAs with
Lipid Modifications in HuH7 Cells

| | Average % | | Average % | |
|---|---|---|---|---|
| | VEGFA mRNA | | VEGFA mRNA | |
| | Remaining | | Remaining | |
| Duplex | (10 nM) | STDEV | (1 nM) | STDEV |
| N3T070127-01 | 100.98 | 3.37 | NA | NA |
| N3T070128-01 | 29.77 | 2.88 | 33.96 | 0.74 |
| N3T070129-01 | 39.5 | 0.24 | 32.17 | 0.80 |
| N3T070130-01 | 33.48 | 1.82 | 37.37 | 2.10 |
| N3T070131-01 | 35.92 | 0.70 | 35.36 | 2.41 |
| N3T070132-01 | 33.82 | 3.69 | 34.63 | 0.86 |
| N3T070133-01 | 38.53 | 0.64 | 33.67 | 0.64 |
| N3T070134-01 | 46.11 | 4.14 | 44.27 | 4.06 |
| N3T070135-01 | 31.32 | 0.84 | 34.12 | 0.37 |
| N3T070136-01 | 32.61 | 2.63 | 39.76 | 0.52 |
| N3T070137-01 | 36.3 | 0.58 | 34.47 | 1.48 |

Compounds N3T070128-01 and N3T070133-01 in Table 9 are tested for their ability to reduce the expression of VEGFA mRNA in wild-type rats. Vehicle (PBS) is used as a control treatment. Brown Norway rats at 6-8 weeks old (3 rats with 6 eyes/group) receive a single intravitreal (IVT) injection of 25 μg of each compound in 2 μL, or a single IVT injection of PBS in 2 μL. 7 days following IVT injection, the rats are euthanized, the eyes removed, and the retinas dissected and prepared for RNA isolation. The level of rat VEGFA mRNA is measured by quantitative real-time PCR. The average percentage expression from 6 eyes is calculated and is shown in Table 11. The data illustrate that N3T070128-01 significantly reduces VEGFA mRNA expression in the retina 7 days following IVT injection.

TABLE 11
In Vivo Activity of Lipid-Conjugated VEGFA siRNAs, 7 Days

| | | Average mRNA remaining | |
|---|---|---|---|
| | Duplex | (PBS group = 100%) | STDEV |
| 1 | N3T070128-01 | 31.6 | 16.1 |
| 2 | N3T070133-01 | 50.3 | 8.1 |

Compounds in Table 9 are tested for their ability to reduce the expression of VEGFA in wild-type rats. Vehicle (PBS) is used as a control treatment. Brown Norway rats at 6-8 weeks old (3 rats with 6 eyes/group) receive a single intravitreal (IVT) injection of 25 μg of each compound in 2 μL, or a single IVT injection of PBS in 2 μL. 14 days following IVT injection, the rats are euthanized, the eyes removed, and the retinas dissected and prepared for RNA isolation. The level of rat VEGFA mRNA is measured by quantitative real-time PCR. The average percentage expression from 6 eyes is calculated and is shown in Table 12. The data illustrate that N3T070128-01, N3T070129-01, and N3T070130-01 significantly reduce VEGFA mRNA expression in the retina 14 days following IVT injection.

TABLE 12
In Vivo Activity of Lipid-Conjugated VEGFA siRNAs, 14 Days

| | | Average mRNA remaining | |
|---|---|---|---|
| | Duplex | (PBS group = 100%) | STDEV |
| 1 | N3T070128-01 | 19.2 | 5 |
| 2 | N3T070129-01 | 39.3 | 16.4 |
| 3 | N3T070130-01 | 42.2 | 16.7 |
| 4 | N3T070131-01 | 66.5 | 15.4 |
| 5 | N3T070132-01 | 69.7 | 14.8 |
| 6 | N3T070133-01 | 76.0 | 36.0 |
| 7 | N3T070134-01 | 64.7 | 23.2 |
| 8 | N3T070135-01 | 54.3 | 19.4 |
| 9 | N3T070136-01 | 81.7 | 15.8 |
| 10 | N3T070137-01 | 74.3 | 16.2 |

### Example 8. Dose Screens of siRNA Duplexes with Lipophilic Moieties

The LRRK2 siRNAs (Table 13) are added to the Human Lung Epithelial cells A549 in a 96-well plate without transfection reagents. All the siRNAs are tested at 10 μM and 3 μM. 72 hours after free uptake, the culture medium is discarded and cells are collected for RNA extraction. Total RNA is extracted by FastPure Cell/Tissue Total RNA Isolation Kit V2 (Vazyme-RC112-01) according to the kit instructions and reverse transcribed to cDNA by HiScript III All-in-one RT SuperMix Perfect for qPCR (Vazyme-R333-01). The cDNA of the target gene is quantified by qPCR with specific primer and probe sets. GAPDH cDNA is measured as a housekeeping gene in parallel. The expression of the target gene in each test sample is determined by relative quantitation (RQ) using the comparative Ct (ΔΔCt) method (Table 14).

TABLE 13
Modified Human LRRK2 siRNAs Conjugated with Lipophilic Moieties

| | SEQ | | SEQ | |
|---|---|---|---|---|
| Duplex | ID NO | sense strand (5′ to 3′) | ID NO | antisense strand (5′ to 3′) |
| N3T130 | 310 | CmsUmsUmGmUm(Uhd)UfGmUfAfUfUmGm | 311 | VPUmsGfsGmAmAmUfUmGmCmAmAmUmA |
| 129-02 | | CmAmAmUmUmCmsCmsAm | | mCfAmAfAmCmAmAmGmsUmsGm |
| N3T130 | 312 | CmsUmsUmGmUmUmUfGmUfAfUfUmGmC | 313 | VPUmsGfsGmAmAmUfUmGmCmAmAmUmA |
| 138-01 | | mAmAmUmUmCmCmsAms[N3T12-012-01] | | mCfAmAfAmCmAmAmGmsUmsGm |

Structures of lipid modifications listed in Table 10 include:

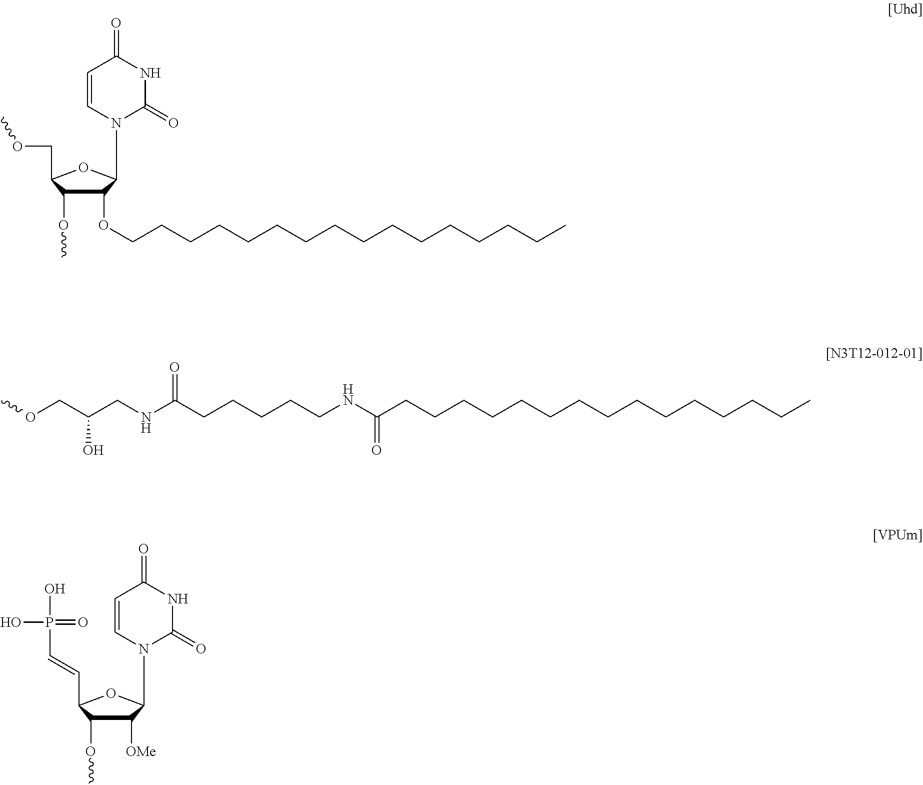

TABLE 14
Human LRRK2 siRNAs with Lipophilic Moieties
in A549 Cells by Free Uptake

| | Average % | | Average % | |
|---|---|---|---|---|
| | LRRK2 mRNA | | LRRK2 mRNA | |
| | Remaining | | Remaining | |
| Duplex | (10 uM) | STDEV | (3 uM) | STDEV |
| N3T130129-02 | 69.6 | 5.8 | 97.2 | 8.9 |
| N3T130138-01 | 33.9 | 2.0 | 46.3 | 2.0 |

The knockdown activity of LRRK2 mRNA is tested at 10 μM and 3 μM by free uptake in A549 cells (Table 14). N3T130138-01 with [N3T12-012-01] modification shows good knockdown activity at both 10 μM and 3 μM.

### Example 9. Dose Screens of ASOs in A549 Cells

The LRRK2 ASOs (Tables 15-16) are added into the Human Lung Epithelial cells A549 in a 96-well plate without transfection reagents. All the ASOs are tested at 10 μM and 3 μM (Table 17). 72 hours after free uptake, the culture medium is discarded and cells are collected for RNA extraction. Total RNA is extracted by FastPure Cell/Tissue Total RNA Isolation Kit V2 (Vazyme-RC112-01) according to the kit instructions and reverse transcribed to cDNA by HiScript III All-in-one RT SuperMix Perfect for qPCR (Vazyme-R333-01). The cDNA of the target gene is quantified by qPCR with specific primer and probe sets. GAPDH cDNA is measured as a housekeeping gene in parallel. The expression of the target gene in each test sample is determined by relative quantitation (RQ) using the comparative Ct (ΔΔCt) method. The results are presented in Table 17.

TABLE 15
Nomenclature for Nucleotides in ASO Agents

| 2′-modified nucleosides | Nomenclature |
|---|---|
| 2′-MOE A | moeA |
| 2′-MOE U | moeU |
| 2′-MOE C | moeC |
| 2′-MOE 5C | moe5C |
| 2′-MOE G | moeG |

TABLE 16
Human LRRK2 ASOs in A549 Cells by Free Uptake

| | Sequences |
|---|---|
| Compound ID | (with modifications, 5′ to 3′) |
| N3T131001-01 | (moeA)*(moe5C)(moeG)(moe5C)(moeA)* |
| (SEQ ID NO: | (d5C)*(dT)*(dT)*(dA)*(dA)*(d5C)* |
| 404) | (dA)*(dA)*(dT)*(dA)*(moeT)(moe5C) |
| | (moeA)*(moeT)*(moeA) |
| N3T131146-01 | (moeA)*(moe5C)(moeG)(moe5C)(moeA)* |
| (SEQ ID NO: | (d5C)*(dT)*(dT)*(dA)*(dA)*(d5C)* |
| 405) | (dA)*(dA)*(dT)*(dA)*(moeT)(moe5C) |
| | (moeA)*(moeT)*(moeA)*[N3T12-012-01] |

Structures of lipid modifications listed in Table 16 include:

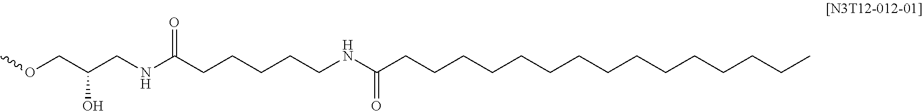

TABLE 17
In Vitro Evaluation of Human LRRK2
ASOs in A549 Cells by Free Uptake

| | Average % | | Average % | |
|---|---|---|---|---|
| | LRRK2 mRNA | | LRRK2 mRNA | |
| | Remaining | | Remaining | |
| Compound ID | (10 uM) | STDEV | (3 uM) | STDEV |
| N3T131001-01 | 30.4 | 0.6 | 82.3 | 5.6 |
| N3T131128-01 | 16.1 | 0.3 | 48.3 | 5 |

N3T131128-01 with [N3T12-012-01] modification shows good knockdown activity.

### Example 10. In Vitro Screening Using Dual Luciferase Reporter Gene Assay

Modifications at the seed region (position 2-8) of the AS strand (from 5′-end) are introduced to reduce off-target effects (Table 18). Modifications in the seed region of the siRNA guide strand enhance siRNA specificity, mitigating off-target effects while still allowing for productive full-length on-target recognition.

TABLE 18
Sense and Antisense Strand Sequences of Human VEGFA dsRNA Duplexes with Off-
target Modifications

| | | | | | Modifications | |
|---|---|---|---|---|---|---|
| | | | | | on antisense | |
| | SEQ | | SEQ | | strand (from 5′ | |
| Duplex | ID NO | sense strand (5′ to 3′) | ID NO | antisense strand (5′ to 3′) | to 3′) | Target |
| N3T120021- | 261 | UGACAAGAAUCCU | 262 | AUUGUGAGGAUUCUUGU | Unmodified | HBV |
| 01 | | CACAAU | | CAAC | control | |
| N3T120022- | 263 | UGACAAGAAUCCU | 264 | AUUGUGIGNA- | Position 7: | HBV |
| 01 | | CACAAU | | A]GGAUUCUUGUCAAC | [GNA-A] | |
| N3T120023- | 265 | UGACAAGAAUCCU | 266 | AUUGUG[N3T1- | Position 7: | HBV |
| 01 | | CACAAU | | A]GGAUUCUUGUCAAC | [N3T1-A] | |
| N3T120024- | 267 | UGACAAGAAUCCU | 268 | AUUG[N3T1- | Position 5: | HBV |
| 01 | | CACAAU | | U]GAGGAUUCUUGUCAAC | [N3T1-U] | |

[GNA-A]: 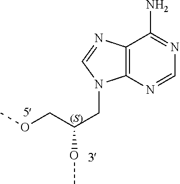
[N3T1-U]: 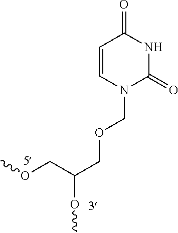
[N3T1-A]:

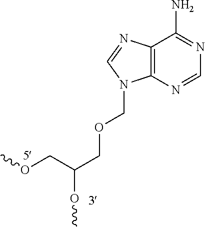

### In Vitro Screening in Dual Luciferase Reporter Gene Assay

293A cells are grown to near-confluence at 37° C. in an atmosphere of 5% CO₂ in Dulbecco's Modified Eagle Medium (DMEM) supplemented with 10% fetal bovine serum (FBS) (Gibco). A single-dose experiment is performed at 50 nM final duplex concentration. Cells are co-transfected in 96-well plates (12000 cells/well) with 100 ng luciferase reporter plasmid (YEASEN) and 50 nM siRNA using 0.4 L/well Lipofectamine 2000 (Thermo Fisher Scientific, CN2541156) according to the manufacturer's instructions, and then incubated at room temperature for 18 minutes. Twenty-four hours after the siRNAs and luciferase reporter plasmid are transfected, Firefly (transfection control) and Renilla (fused to target sequence) luciferase are measured. siRNA activity is determined by normalizing the Renilla (target sequence) signal to the Firefly (control) signal within each well (Table 19). The specific insert sequences are as follows:

[SEQ ID NO: 269]
GTCGCATATAATATTACATAAATAAAAGTCGCATATAATATTACATAAA
TAAAAGTCGCATA

The results are presented in Tables 19-21.

TABLE 19
In Vitro Dual Luciferase 50 nM Screen
with Off-Target Modified siRNAs

| | | % of Renilla | |
|---|---|---|---|
| | Modifications on | Luciferase | |
| | antisense strand | signal | |
| Duplex | (from 5′ to 3′) | Remaining | STDEV |
| N3T120021-01 | Unmodified control | 15.55 | 3.44 |
| N3T120022-01 | Position 7: [GNA-A] | 86.42 | 14.74 |
| N3T120023-01 | Position 7: [N3T1-A] | 60.54 | 2.54 |
| N3T120024-01 | Position 5: [N3T1-U] | 81.10 | 2.64 |

The off-target effects are largely driven by binding of the RISC-loaded siRNA to off-target transcripts mediated through base pairing between the seed region of the siRNA guide strand (nucleotides 2-8) and complementary site(s) in the mRNAs. Thermally destabilizing modifications in the seed region of the siRNA guide strand enhance siRNA specificity, mitigating off-target effects while still allowing for productive full-length on-target recognition. N3T120021-01 with no modifications has very strong Renilla Luciferase signal inhibition. When N3T120022-01, N3T120023-01, and N3T120024-01 are co-transfected with luciferase reporter plasmid, Renilla Luciferase signal is improved significantly, which indicates that [N3T12-027-01] and [N3T12-031-01] modifications can mitigate off-target effects.

In order to observe how(S)/(R)-enantiomers affects the Renilla Luciferase signal, 14 siRNAs are synthesized, as in Table 20.

HEK-293a cells are grown to near confluence at 37° C. in an atmosphere of 5% CO₂ in Dulbecco's Modified Eagle Medium (DMEM) supplemented with 10% fetal bovine serum (FBS) (Gibco). Cells are co-transfected in 96-well plates (8000 cells/well) with 10 ng luciferase reporter plasmid (YEASEN) and siRNAs using 0.4 μL/well Lipofectamine 2000 (Thermo Fisher Scientific) according to the manufacturer's instructions, and then incubated at room temperature for 18 minutes. 24 hours after the siRNAs and luciferase reporter plasmid are transfected, Firefly (transfection control) and Renilla (fused to target sequence) luciferase are measured (Table 21). siRNA activity is determined by normalizing the Renilla (target sequence) signal to the Firefly (control) signal within each well. The on-target/off-target reporter plasmid contained the following sequences in the 3′-UTR of Renilla luciferase. The specific insert sequences are as follows:

HBV on-target:
[SEQ ID NO: 270]
5′ GTTGACAAGAATCCTCACAAT3′
HBV off-target:
[SEQ ID NO: 271]
5′TGGTCACCTCCGACTCACAAT****TGGTCACCTCCGACTCACAAT
****TGGTCACCTCCGACTCACAAT****TGGTCACCTCCGACTCACA
AT****TGGTCACCTCCGACTCACAAT3′
VEGFA on-target″
[SEQ ID NO: 272]
5′TGTGAATGCAGACCAAAGAAA3′
VEGFA off-target:
[SEQ ID NO: 273]
5′GTGTCCGTACTCACAAAGAAA****GTGTCCGTACTCACAAAGAAA
****GTGTCCGTACTCACAAAGAAA****GTGTCCGTACTCACAAAGA
AA****GTGTCCGTACTCACAAAGAAA3′
****: Special nucleotide DNA linker 20240724SS14
from Suzhou Biosyntech

The results are presented in Table 21.

TABLE 20
siRNAs with Off-Target Modifications

| | | | | | Modifica- | |
|---|---|---|---|---|---|---|
| | | | | | tions | |
| | SEQ | | SEQ | | on antisense | |
| | ID | | ID | | strand (from | |
| Duplex | NO | sense strand (5′ to 3′) | NO | antisense strand (5′ to 3′) | 5′ to 3′) | Targets |
| N3T120021- | 342 | UGACAAGAAUCCUCAC | 343 | AUUGUGAGGAUUCU | Unmodified | HBV |
| 02 | | AAU | | UGUCAAC | control | |
| N3T120022- | 344 | UGACAAGAAUCCUCAC | 345 | AUUGUG[GNA- | Position 7: | HBV |
| 02 | | AAU | | A]GGAUUCUUGUCAA | [GNA-A] | |
| | | | | C | | |
| N3T120039- | 346 | UGACAAGAAUCCUCAC | 347 | AUUGUG[N3T1-A- | Position 7: | HBV |
| 01 | | AAU | | SIGGAUUCUUGUCAA | [N3T1-A-S] | |
| | | | | C | | |
| N3T120040- | 348 | UGACAAGAAUCCUCAC | 349 | AUUGUG[N3T1-A- | Position 7: | HBV |
| 01 | | AAU | | RIGGAUUCUUGUCAA | [N3T1-A-R] | |
| | | | | C | | |
| N3T120041- | 350 | UGACAAGAAUCCUCAC | 351 | AUUG[GNA- | Position 5: | HBV |
| 01 | | AAU | | U]GAGGAUUCUUGUC | [GNA-U] | |
| | | | | AAC | | |
| N3T120042- | 352 | UGACAAGAAUCCUCAC | 353 | AUUG[N3T1-U- | Position 5: | HBV |
| 01 | | AAU | | S]GAGGAUUCUUGUC | [N3T1-U-S] | |
| | | | | AAC | | |
| N3T120043- | 354 | UGACAAGAAUCCUCAC | 355 | AUUG[N3T1-U- | Position 5: | HBV |
| 01 | | AAU | | R]GAGGAUUCUUGUC | [N3T1-U-R] | |
| | | | | AAC | | |
| N3T120044- | 356 | UGAAUGCAGACCAAAG | 357 | UUUCUUUGGUCUGCA | \|Unmodified | VEGFA |
| 01 | | AAA | | UUCACA | control | |
| N3T120045- | 358 | UGAAUGCAGACCAAAG | 359 | UUUCUU[GNA- | Position 7: | VEGFA |
| 01 | | AAA | | U]GGUCUGCAUUCAC | [GNA-U] | |
| | | | | A | | |
| N3T120046- | 360 | UGAAUGCAGACCAAAG | 361 | UUUCUU[N3T1-U- | Position 7: | VEGFA |
| 01 | | AAA | | SIGGUCUGCAUUCAC | [N3T1-U-S] | |
| | | | | A | | |
| N3T120047- | 362 | UGAAUGCAGACCAAAG | 363 | UUUCUU[N3T1-U- | Position 7: | VEGFA |
| 01 | | AAA | | RIGGUCUGCAUUCAC | [N3T1-U-R] | |
| | | | | A | | |
| N3T120048- | 364 | UGAAUGCAGACCAAAG | 365 | UUUC[GNA- | Position 5: | VEGFA |
| 01 | | AAA | | UJUUGGUCUGCAUUC | [GNA-U] | |
| | | | | ACA | | |
| N3T120049- | 366 | UGAAUGCAGACCAAAG | 367 | UUUC[N3T1-U- | Position 5: | VEGFA |
| 01 | | AAA | | SJUUGGUCUGCAUUC | [N3T1-U-S] | |
| | | | | ACA | | |
| N3T120050- | 368 | UGAAUGCAGACCAAAG | 369 | UUUC[N3T1-U- | Position 5: | VEGFA |
| 01 | | AAA | | RJUUGGUCUGCAUUC | [N3T1-U-R] | |
| | | | | ACA | | |

[GNA-A]: 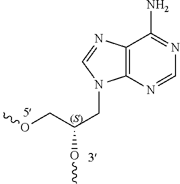
[GNA-U]: 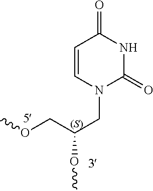
[N3T1-U-R]: 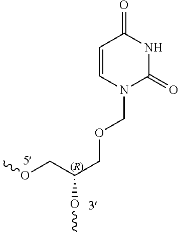
[N3T1-U-S]: 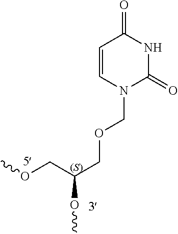
[N3T1-A-R]: 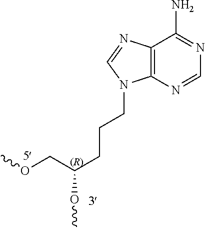
[N3T1-A-S]:

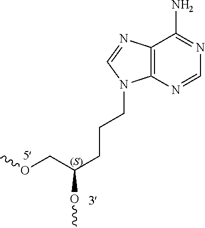

TABLE 21
Evaluating On- and Off-Target siRNAs using Dual Luciferase Reporter Assay

| | | On-target | Off-target | |
|---|---|---|---|---|
| | | (Relative | (Relative | |
| | Modifications on | Ren/Fir | Ren/Fir | |
| | antisense strand | Expression %) | Expression %) | |
| | (from 5′ to 3′) | 1 nM | 50 nM | 10 nM |
| N3T120021-02 | Unmodified control | 8.6 | 7.3 | 6.3 |
| N3T120022-02 | Position 7: [GNA-A] | 4.3 | 46.4 | 38.4 |
| N3T120039-01 | Position 7: [N3T1-A-S] | 4.7 | 18.0 | 14.5 |
| N3T120040-01 | Position 7: [N3T1-A-R] | 2.8 | 6.5 | 9.5 |
| N3T120041-01 | Position 5: [GNA-U] | 6.4 | 9.1 | 11.0 |
| N3T120042-01 | Position 5: [N3T1-U-S] | 4.7 | 27.2 | 29.4 |
| N3T120043-01 | Position 5: [N3T1-U-R] | 5.2 | 72.9 | 63.6 |
| N3T120044-01 | Unmodified control | 11.9 | 21.3 | 11.3 |
| N3T120045-01 | Position 7: [GNA-U] | 4.6 | 138.3 | 135.8 |
| N3T120046-01 | Position 7: [N3T1-U-S] | 9.2 | 76.7 | 52.9 |
| N3T120047-01 | Position 7: [N3T1-U-R] | 13.7 | 49.5 | 43.3 |
| N3T120048-01 | Position 5: [GNA-U] | 14.4 | 16.1 | 18.6 |
| N3T120049-01 | Position 5: [N3T1-U-S] | 8.7 | 30.9 | 29.5 |
| N3T120050-01 | Position 5: [N3T1-U-R] | 6.3 | 55.3 | 41.9 |

Most(S)/(R)-enantiomers can increase off-target Renilla Luciferase signal inhibition at both 50 nM and 10 nM concentrations while keeping full-length on-target inhibition at 1 nM.

### Example 11. In Vitro Evaluation of ANGPT2 siRNAs by Transfection with Off-Target Modifications

The siRNAs (Table 22) are transfected into RMG-I cells with RNAiMAX (Invitrogen, 13778075) in a 48-well plate. All the siRNAs are tested at 1 nM and 0.1 nM (Table 23). 48 hours after transfection, the culture medium is discarded and cells are collected for RNA extraction. Total RNA is extracted by FastPure Cell/Tissue Total RNA Isolation Kit V2 (Vazyme-RC112-01) according to the kit instructions, and reverse transcribed to cDNA by HiScript IV All-in-One Ultra RT SuperMix for qPCR (Vazyme-R433-01). The cDNA of target gene is quantified by qPCR with specific primer and probe sets. GAPDH cDNA is measured as a housekeeping gene in parallel. 14 μL PCR mixture and 6 μL sample cDNA (5-fold dilution) are added to 96-well PCR plates. The PCR is performed with 37° C. for 2 minutes, 95° C. for 5 minutes, then cycling at 95° C. for 10 seconds and 60° C. for 30 seconds for 40 cycles. The results are presented in Table 23.

TABLE 22
Human ANGPT2 siRNAs with Off-Target Modifications

| | SEQ | | SEQ | | Modifications on |
|---|---|---|---|---|---|
| | ID | sense | ID | antisense | antisense strand |
| Duplex | NO | strand (5′ to 3′) | NO | strand (5′ to 3′) | (from 5′ to 3′) |
| N3T120065- | 370 | GmsUms AmAmAfUmAfAfCfU | 371 | VPUmsUfsGmUmUmUfUmCfCf | Unmodified control |
| 01 | | mGmGmAmAmAmAmCmAms | | AmGmUmUmAfUmUfUmAmCm | |
| | | Ams[C5Cholesterol] | | sUmsGm | |
| N3T120066- | 372 | GmsUmsAmAmAfUmAfAfCfU | 373 | VPUmsUfsGmUm[N3T1-U- | Position 5: |
| 01 | | mGmGmAmAmAmAmCmAms | | R]UfUmCfCfAmGmUmUmAfUm | [N3T1-U-R] |
| | | Ams[C5Cholesterol] | | UfUmAmCmsUmsGm | |
| N3T120067 | 374 | GmsUmsAmAmAfUmAfAfCfU | 375 | VPUmsUfsGmUmUmUf[N3T1- | Position 7: |
| 01 | | mGmGmAmAmAmAmCmAms | | U- | [N3T1-U-R] |
| | | Ams[C5Cholesterol] | | R]CfCfAmGmUmUmAfUmUfUm | |
| | | | | AmCmsUmsGm | |
| N3T120107- | 376 | GmsUmsAmAmAfUmAfAfCfU | 377 | VPUmsUfsGmUmUmf[GNA- | Position 7: |
| 01 | | mGmGmAmAmAmAmCmAms | | U]CfCfAmGmUmUmAfUmUfUm | [GNA-U] |
| | | Ams[C5Cholesterol] | | AmCmsUmsGm | |
| N3T120068- | 378 | AmsUmsGmUmGfGmUfCfCfU | 379 | VPUmsAfsAmGmUmUfGmGfAf | Unmodified control |
| 01 | | mUmCmCmAmAmCmUmUms | | AmGmGmAmCfCmAfCmAmUm | |
| | | Ams[C5Cholesterol] | | sGmsCm | |
| N3T120069- | 380 | AmsUmsGmUmGfGmUfCfCfU | 381 | VPUmsAfsAmGm[N3T1-U- | Position 5: |
| 01 | | mUmCmCmAmAmCmUmUms | | R]UfGmGfAfAmGmGmAmCfCm | [N3T1-U-R] |
| | | Ams[C5Cholesterol] | | AfCmAmUmsGmsCm | |
| N3T120070- | 382 | AmsUmsGmUmGfGmUfCfCfU | 383 | VPUmsAfsAmGmUmUf[N3T1- | Position 7: |
| 01 | | mUmCmCmAmAmCmUmUms | | G- | [N3T1-G-R] |
| | | Ams[C5Cholesterol] | | R]GfAfAmGmGmAmCfCmAfCm | |
| | | | | AmUmsGmsCm | |
| N3T120108- | 384 | AmsUmsGmUmGfGmUfCfCfU | 385 | VPUms AfsAmGmUmUf[GNA- | Position 7: |
| 01 | | mUmCmCmAmAmCmUmUms | | G]GfAfAmGmGmAmCfCmAfCm | [GNA-G] |
| | | Ams[C5Cholesterol] | | AmUmsGmsCm | |
| N3T120071- | 386 | AmsUmsCmAmAfGmGfCfCfU | 387 | VPUmsGfsUmCmAmCfAmGfUf | Unmodified control |
| 01 | | mAmCmUmGmUmGmAmCms | | AmGmGmCmCfUmUfGmAmUm | |
| | | Ams[C5Cholesterol] | | sCmsUm | |
| N3T120072- | 388 | AmsUmsCmAmAfGmGfCfCfU | 389 | VPUmsGfsUmCm[N3T1-A- | Position 5: |
| 01 | | mAmCmUmGmUmGmAmCms | | R]CfAmGfUfAmGmGmCmCfUm | [N3T1-A-R] |
| | | Ams[C5Cholesterol] | | UfGmAmUmsCms Um | |
| N3T120073- | 390 | AmsUmsCmAmAfGmGfCfCfU | 391 | VPUmsGfsUmCmAmCf[N3T1-A- | Position 7: |
| 01 | | mAmCmUmGmUmGmAmCms | | R]GfUfAmGmGmCmCfUmUfGm | [N3T1-A-R] |
| | | Ams[C5Cholesterol] | | AmUmsCmsUm | |
| N3T120109- | 392 | AmsUmsCmAmAfGmGfCfCfU | 393 | VPUmsGfsUmCmAmCf[GNA- | Position 7: |
| 01 | | mAmCmUmGmUmGmAmCms | | A]GfUfAmGmGmCmCfUmUfGm | [GNA-A] |
| | | Ams[C5Cholesterol] | | AmUmsCmsUm | |

[C5Cholesterol]: 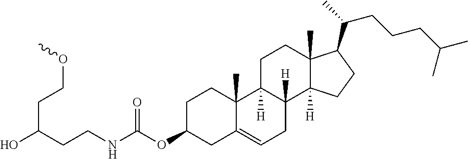
[N3T1-U-R]: 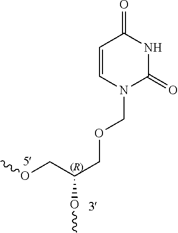
[N3T1-A-R]: 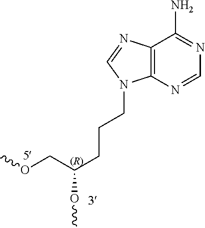
[N3T1-G-R]: 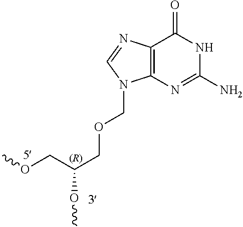
[GNA-U]: 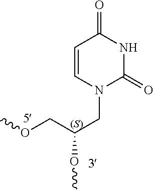
[GNA-A]: 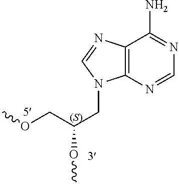
[GNA-G]: 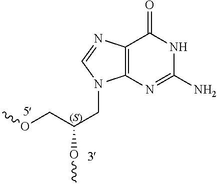
[VPUm]: 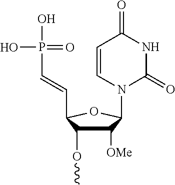
Am, Cm, Gm, Um indicate 2′-O-methyl (2′-OMe) sugar modifications, respectively, to adenosine, cytidine, guanosine and uridine;
Af, Cf, Gf, Uf indicate 2′-deoxy-2-fluoro (2′-F) sugar modifications, respectively, to adenosine, cytidine, guanosine and uridine;
s indicates phosphorothioate (PS) linkage.

TABLE 23
In Vitro Screening of Human ANGPT2 siRNAs by Transfection

| | | Average % | | Average % | |
|---|---|---|---|---|---|
| | Modifications on | ANGPT2 mRNA | | ANGPT2 mRNA | |
| | antisense strand | Remaining | | Remaining | |
| Duplex | (from 5′ to 3′) | (1 nM) | STDEV | (0.1 nM) | STDEV |
| N3T120065-01 | Unmodified control | 23 | 0.31 | 51.3 | 7.46 |
| N3T120066-01 | Position 5: [N3T1-U-R] | 34.8 | 2.44 | 51.9 | 3.84 |
| N3T120067-01 | Position 7: [N3T1-U-R] | 19.7 | 2.72 | 51.8 | 5.14 |
| N3T120107-01 | Position 7: [GNA-U] | 24.8 | 0.37 | 51 | 4.48 |
| N3T120068-01 | Unmodified control | 13.9 | 0.00 | 43.4 | 2.67 |
| N3T120069-01 | Position 5: [N3T1-U-R] | 23 | 2.07 | 65.9 | 5.87 |
| N3T120070-01 | Position 7: [N3T1-G-R] | 12.4 | 1.62 | 56.5 | 0.13 |
| N3T120108-01 | Position 7: [GNA-G] | 20.2 | 1.75 | 56.5 | 1.48 |
| N3T120071-01 | Unmodified control | 29.5 | 0.15 | 72.2 | 5.03 |
| N3T120072-01 | Position 5: [N3T1-A-R] | 51.4 | 5.80 | 78.4 | 12.38 |
| N3T120073-01 | Position 7: [N3T1-A-R] | 47.7 | 2.89 | 95 | 3.41 |
| N3T120109-01 | Position 7: [GNA-A] | 47.3 | 5.92 | 96.8 | 8.96 |

### Example 12. In Vitro Evaluation of Human LRRK2 siRNAs with Off-Target Modifications by Transfection

The siRNAs (Table 24) are transfected into the Human Lung Epithelial cells A549 with RNAiMAX (Invitrogen, 13778075) in a 96-well plate. All the siRNAs are tested at 1 nM and 0.1 nM (Table 25). 48 hours after transfection, the culture medium is discarded and cells are collected for RNA extraction. Total RNA is extracted by FastPure Cell/Tissue Total RNA Isolation Kit V2 (Vazyme-RC112-01) according to the kit instructions and reversed transcribed to cDNA by HiScript III All-in-one RT SuperMix Perfect for qPCR (Vazyme-R333-01). The cDNA of the target gene is quantified by qPCR with specific primer and probe sets. GAPDH cDNA is measured as a housekeeping gene in parallel. The expression of the target gene in each test sample is determined by relative quantitation (RQ) using the comparative Ct (ΔΔCt) method. The results are presented in Table 25.

TABLE 24
Human LRRK2 siRNAs with Off-Target Modifications
on antisense

| | | | | | Modifications |
|---|---|---|---|---|---|
| | | | | | on antisense |
| | SEQ ID | | SEQ ID | | strand (from 5′ |
| Duplex | NO | sense strand (5′ to 3′) | NO | antisense strand (5′ to 3′) | to 3′) |
| N3T120056- | 314 | GmsUmsGmGmCmAmAfsUmGfUf | 315 | VPUmsAfsUmAmAmCfAmCmC | Unmodified |
| 01 | | CfAmGmGmUmGmUmUmAmUms | | mUmGmAmCmAfUmUfGmCmC | control |
| | | Ams[N3T12-012-01] | | mAmCmsUmsCm | |
| N3T120057- | 316 | GmsUmsGmGmCmAmAfsUmGfUf | 317 | VPUmsAfsUmAm[N3T1-A- | Position 5: |
| 01 | | CfAmGmGmUmGmUmUmAmUms | | R]CfAmCmCmUmGmAmCmAfU | [N3T1-A-R] |
| | | Ams[N3T12-012-01] | | mUfGmCmCmAmCmsUmsCm | |
| N3T120058- | 318 | GmsUmsGmGmCmAmAfsUmGfUf | 319 | VPUmsAfsUmAmAmCf[N3T1-A- | Position 7: |
| 01 | | CfAmGmGmUmGmUmUmAmUms | | R]CmCmUmGmAmCmAfUmUfG | [N3T1-A-R] |
| | | Ams[N3T12-012-01] | | mCmCmAmCmsUmsCm | |
| N3T120059- | 320 | UmsUmsGmGmAmCmUfUmGfCf | 321 | VPUms UfsUmAmUmUfAmCmU | Unmodified |
| 01 | | AfCmAmGmUmAmAmUmAmAms | | mGmUmGmCmAfAmGfUmCmC | control |
| | | Ams[N3T12-012-01] | | mAmAmsAmsUm | |
| N3T120060- | 322 | UmsUmsGmGmAmCmUfUmGfCf | 323 | VPUms UfsUmAm[N3T1-U- | Position 5: |
| 01 | | AfCmAmGmUmAmAmUmAmAms | | R]UfAmCmUmGmUmGmCmAfA | [N3T1-U-R] |
| | | Ams[N3T12-012-01] | | mGfUmCmCmAmAmsAmsUm | |
| N3T120061- | 324 | UmsUmsGmGmAmCmUfUmGfCf | 325 | VPUmsUfsUmAmUmUf[N3T1- | Position 7: |
| 01 | | AfCmAmGmUmAmAmUmAmAms | | A- | [N3T1-A-R] |
| | | Ams [N3T12-012-01] | | R]CmUmGmUmGmCmAfAmGfU | |
| | | | | mCmCmAmAmsAmsUm | |
| N3T120062- | 326 | UmsAmsGmAmUmAmUfGmAfGf | 327 | VPUmsAfsUmCmAmUfUmGmC | Unmodified |
| 01 | | CfAmGmCmAmAmUmGmAmsUm | | mUmGmCmUmCfAmUfAmUmC | control |
| | | Ams[N3T12-012-01] | | mUmAmsAmsAm | |
| N3T120063- | 328 | Ums AmsGmAmUmAmUfGmAfGf | 329 | VPUmsAfsUmCm[N3T1-A- | Position 5: |
| 01 | | CfAmGmCmAmAmUmGmAmsUm | | R]UfUmGmCmUmGmCmUmCfA | [N3T1-A-R] |
| | | Ams[N3T12-012-01] | | mUfAmUmCmUmAmsAmsAm | |
| N3T120064- | 330 | UmsAmsGmAmUmAmUfGmAfGf | 331 | VPUmsAfsUmCmAmUf[N3T1-U- | Position 7: |
| 01 | | CfAmGmCmAmAmUmGmAmsUm | | R]GmCmUmGmCmUmCfAmUfA | [N3T1-U-R] |
| | | Ams[N3T12-012-01] | | mUmCmUmAmsAmsAm | |

[N3T12-012-01]: 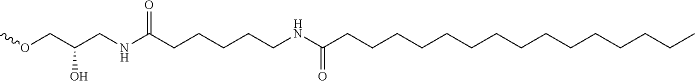
[N3T1-U-R]: 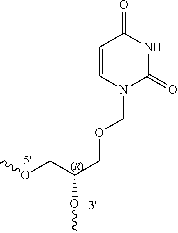
[N3T1-A-R]: 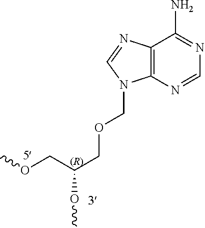
[VPUm]: 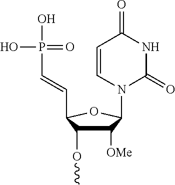
Am, Cm, Gm, Um indicate 2′-O-methyl (2′-OMe) sugar modifications, respectively, to adenosine, cytidine, guanosine and uridine;
Af, Cf, Gf, Uf indicate 2′-deoxy-2′-fluoro (2′-F) sugar modifications, respectively, to adenosine, cytidine, guanosine and uridine;
s indicates phosphorothioate (PS) linkage.

TABLE 25
In Vitro Screening of Human LRRK2 siRNAs by Transfection

| | | Average % | | Average % | |
|---|---|---|---|---|---|
| | Modifications on | LRRK2 mRNA | | LRRK2 mRNA | |
| | antisense strand | Remaining | | Remaining | |
| Compound ID | (from 5′ to 3′) | (1 nM) | STDEV | (0.1 nM) | STDEV |
| N3T120056-01 | Unmodified control | 19.9 | 1.5 | 32.4 | 13.5 |
| N3T120057-01 | Position 5: [N3T1-A-R] | 33.8 | 0.3 | 72.7 | 8.7 |
| N3T120058-01 | Position 7: [N3T1-A-R] | 60.8 | 9.9 | 93.7 | 8.0 |
| N3T120059-01 | Unmodified control | 12.6 | 3.5 | 21.3 | 0.8 |
| N3T120060-01 | Position 5: [N3T1-U-R] | 14.4 | 1.4 | 21.3 | 1.7 |
| N3T120061-01 | Position 7: [N3T1-A-R] | 18.0 | 3.1 | 26.8 | 2.5 |
| N3T120062-01 | Unmodified control | 14.9 | 0.6 | 32.7 | 1.5 |
| N3T120063-01 | Position 5: [N3T1-A-R] | 20.7 | 1.4 | 46.1 | 0.3 |
| N3T120064-01 | Position 7: [N3T1-U-R] | 31.4 | 0.0 | 62.3 | 6.8 |

Modifications on N3T120059-01 with [N3T1-U—R] at position 5 of antisense strand or [N3T1-A-R] at position 7 of antisense strand can keep LRRK2 mRNA knockdown activity.

### Example 13. In Vivo Study of Human VEGFA siRNAs and ANGPT2 siRNAs in Sprague Dawley Rats

Compounds N3T070128-03, N3T120055-01, N3T120068-01, and N3T120070-01 in Table 26 are tested for their ability to reduce the expression of VEGFA and ANGPT2 in Sprague Dawley Rats. Vehicle (PBS) is used as a control treatment. Sprague Dawley Rats at 6-8 weeks old (3 rats with 6 eyes/group) receive a single intravitreal (IVT) injection of 10 μg of each compound in 2 μL, or a single IVT injection of PBS in 2 μL. 14 days following IVT injection, the rats are euthanized, the eyes removed, and the retinas dissected and prepared for RNA isolation. The level of rat VEGFA and ANGPT2 mRNA is measured by quantitative real-time PCR. The average percentage expression from 6 eyes is calculated and shown in Tables 27 and 28.

TABLE 26
Human VEGFA siRNAs and ANGPT2 siRNAs for In Vivo Study

| | SEQ | | SEQ | | |
|---|---|---|---|---|---|
| Duplex | ID NO | sense strand (5′ to 3′) | ID NO | antisense strand (5′ to 3′) | Target |
| N3T070128-03 | 394 | AmsGmsAmGmAfAmAfAf | 395 | VPUmsAfsCmAmCmUfUmUfCfUm | VEGFA |
| | | GfAmGmAmAmAmGmUm | | CmUmUmUfUmCfUmCmUmsUms | |
| | | GmUmsUms[C5Cholesterol] | | Um | |
| N3T120055-01 | 396 | AmsGmsAmGmAfAmAfAf | 397 | VPUmsAfsCmAmCmUf[N3T1-U- | VEGFA |
| | | GfAmGmAmAmAmGmUm | | R]UfCfUmCmUmUmUfUmCfUmC | |
| | | GmUmsUms[C5Cholesterol] | | mUmsUmsUm | |
| N3T120068-01 | 398 | AmsUmsGmUmGfGmUfCfCfU | 399 | VPUmsAfsAmGmUmUfGmGfAfAm | ANGPT2 |
| | | mUmCmCmAmAmCmUmUms | | GmGmAmCfCmAfCmAmUmsGms | |
| | | Ams(C5Cholesterol) | | Cm | |
| N3T120070-01 | 400 | AmsUmsGmUmGfGmUfCfCfU | 401 | VPUmsAfsAmGmUmUf[N3T1-G- | ANGPT2 |
| | | mUmCmCmAmAmCmUmUms | | R]GfAfAmGmGmAmCfCmAfCmA | |
| | | Ams(C5Cholesterol) | | mUmsGmsCm | |

TABLE 27
In Vivo Activity of Human VEGFA siRNAs, 14 Days

| | | Average mRNA remaining | |
|---|---|---|---|
| | Compound ID | (PBS group = 100%) | STDEV |
| 1 | N3T070128-03 | 70.1 | 17.6 |
| 2 | N3T120055-01 | 50.1 | 18.1 |

TABLE 28
In Vivo Activity of Human ANGPT2 siRNAs, 14 Days

| | | Average mRNA remaining | |
|---|---|---|---|
| | Compound ID | (PBS group = 100%) | STDEV |
| 1 | N3T120068-01 | 41.4 | 15.6 |
| 2 | N3T120070-01 | 57.7 | 21.4 |

N3T070128-03 and N3T120068-01 with off target modifications can keep knockdown activity in vivo.

### Example 14. In Vivo Study of Human LRRK2 siRNAs in C57BL/6J-Tg (LRRK2*G2019S)2AMjff/J Mice

Human LRRK2 siRNAs N3T130138-01, N3T130146-01, N3T130148-01 and N3T130149-01 in Table 29 are tested for their ability to reduce the expression of LRRK2 mRNA in male C57BL/6J-Tg (LRRK2*G2019S)2AMjff/J mice (2-3 mice/group). Vehicle (PBS) is used as a control treatment. 300 μg of each siRNA in 10 μL is administered bilaterally (5 μL per ventricle) into the lateral ventricles of mice. On the fourteenth day (Day 14) post-administration, mice are euthanized. The cortex is collected and stored at −80° C. until further analysis.

TABLE 29
Human LRRK2 siRNAs for In Vivo Study

| | SEQ ID | | SEQ ID | |
|---|---|---|---|---|
| Duplex | NO | sense strand (5′ to 3′) | NO | antisense strand (5′ to 3′) |
| N3T130129- | 332 | CmsUmsUmGmUm(Uhd) UfGmUfAfUfU | 333 | VPUmsGfsGmAmAmUfUmGmCmA |
| 02 | | mGmCmAmAmUmUmCmsCmsAm | | mAmUmAmCfAmAfAmCmAmAm |
| | | | | GmsUmsGm |
| N3T130138- | 334 | CmsUmsUmGmUmUmUfGmUfAfUfUmG | 335 | VPUmsGfsGmAmAmUfUmGmCmA |
| 01 | | mCmAmAmUmUmCmCmsAms[N3T12- | | mAmUmAmCfAmAfAmCmAmAm |
| | | 012-01] | | GmsUmsGm |
| N3T130146- | 336 | UmsUmsGmGmAmCmUfUmGfCfAfCmA | 337 | VPUms UfsUmAmUmUfAmCmUmG |
| 01 | | mGmUmAmAmUmAmAmsAms[N3T12- | | mUmGmCmAfAmGfUmCmCmAm |
| | | 012-01] | | AmsAmsUm |
| N3T130148- | 338 | CmsAmsGmGmUmCmGfUmGfAfGfGmA | 339 | VPUmsAfsUmAmGmAfAmUmUmC |
| 01 | | mAmUmUmCmUmAmUmsAms[N3T12- | | mCmUmCmAfCmGfAmCmCmUmG |
| | | 012-01] | | msCmsAm |
| N3T130149- | 340 | UmsAmsGmAmUmAmUfGmAfGfCfAmG | 341 | VPUmsAfsUmCmAmUfUmGmCmU |
| 01 | | mCmAmAmUmGmAmUmsAms[N3T12- | | mGmCmUmCfAmUfAmUmCmUm |
| | | 012-01] | | AmsAmsAm |

[N3T12-012-01] 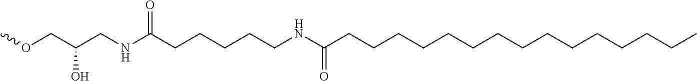
[VPUm]: 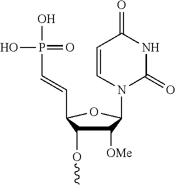
Am, Cm, Gm, Um indicate 2′-O-methyl (2′-OMe) sugar modifications, respectively, to adenosine, cytidine, guanosine and uridine;
Af, Cf, Gf, UF indicate 2′-deoxy-2′-fluoro (2′-F) sugar modifications, respectively, to adenosine, cytidine, guanosine and uridine;
s indicates phosphorothioate (PS) linkage.

The level of LRRK2 mRNA is quantified by real-time PCR and the results are presented in Table 30. Compared to the N3T070129-02 (same sequence and C16 lipid conjugation as AD-1807334 in WO2023278607A1, the contents of which are herein incorporated to the fullest extent allowable by law), N3T130138-01, N3T070146-02 and N3T070149-01 have better knockdown activity in vivo.

TABLE 30
In Vivo Activity of Human LRRK2 siRNAs, 14 Days

| | | Average mRNA remaining | |
|---|---|---|---|
| | Duplex | (PBS group = 100%) | STDEV |
| 1 | N3T070129-02 | 89.0 | 33.9 |
| 2 | N3T070138-01 | 83.0 | 14.1 |
| 3 | N3T070146-02 | 53.7 | 6.4 |
| 4 | N3T070149-01 | 70.3 | 7.6 |

The level of Lrrk2 protein and phosphorylated Lrrk2 (p-Lrrk2) which is related to Parkinson's disease are measured by Western blot and the average percentage expression is calculated (Table 31 and Table 32).

TABLE 31
In Vivo Activity of Human LRRK2 siRNAs, 14 Days

| | | Average Lrrk2 protein remaining | |
|---|---|---|---|
| | Duplex | (PBS group = 100%) | STDEV |
| 1 | N3T070138-01 | 33.2 | 6.4 |
| 2 | N3T070146-01 | 74.2 | 8.5 |
| 3 | N3T070148-01 | 48.6 | 14.7 |
| 4 | N3T070149-01 | 77.2 | 9.9 |

TABLE 32
In Vivo Activity of Human LRRK2 siRNAs, 14 Days

| | | Average p-Lrrk2 protein remaining | |
|---|---|---|---|
| | Duplex | (PBS group = 100%) | STDEV |
| 1 | N3T070138-01 | 31.0 | 7.0 |
| 2 | N3T070146-01 | 57.4 | 12.2 |
| 3 | N3T070148-01 | 24.6 | 7.2 |
| 4 | N3T070149-01 | 64.0 | 9.8 |

N3T130138-01, N3T070146-01, N3T070148-01and N3T070149-01 have good knockdown activity of Lrrk2 protein in vivo.

### Example 15: RISC Loading Modifications at Position 1 of the Antisense Strand

The RNA-induced silencing complex, or RISC, is a ribonucleoprotein complex that loads the antisense strand of the dsRNA duplex. Once loaded, the antisense RNA on the RISC recognizes complementary mRNA, whereupon one of the proteins in RISC, Argonaute, is activated and cleaves the mRNA, resulting in degradation of the mRNA and silencing of gene expression. Modified nucleosides and analogs (e.g., Scheme 21) at a terminal position on the antisense strand can enhance loading of the RNA to RISC by binding to the effector protein of RISC, Argonaute 2 (AGO2).

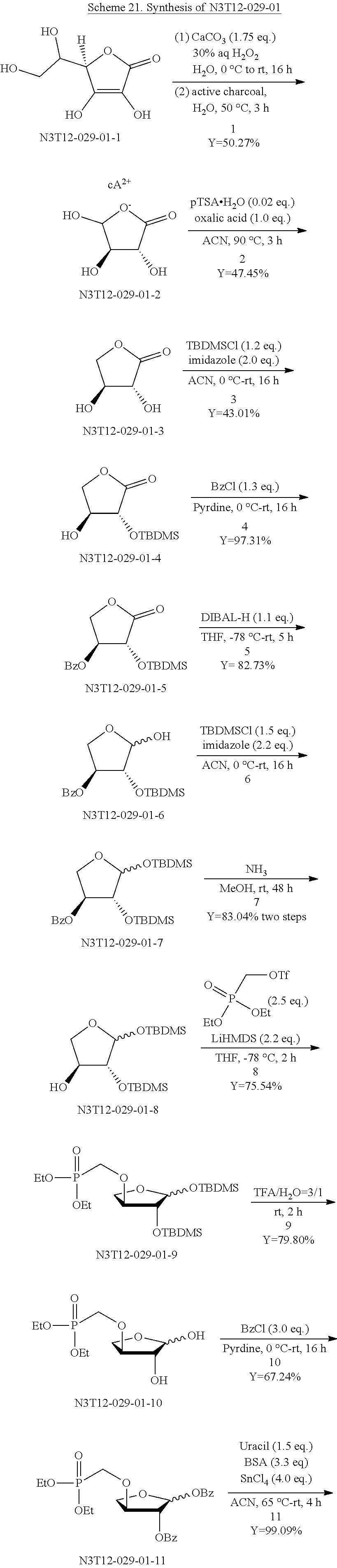

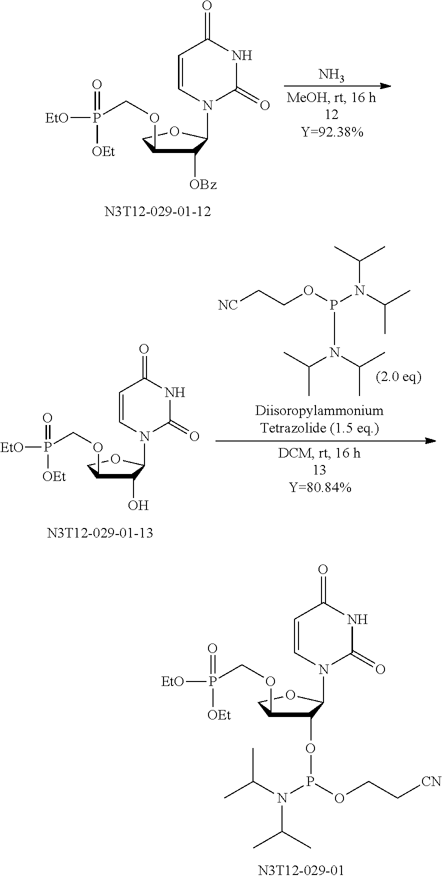

N3T12-029-01-2: To a cold (0° C.) solution containing N3T12-029-01-1 (50 g, 283.90 mmol) in water (400 mL), calcium carbonate (49.73 g, 496.82 mmol) is added slowly over 10 minutes (evolution of CO₂ is observed) at 0° C. To the resulting heterogeneous slurry is added 30% H₂O₂ (100 mL) dropwise over a period of 1 hour at 0° C. The reaction mixture is allowed to warm to RT and stirred overnight. The mixture is filtered, and the filter cake is washed by water (3*50 mL). The filtrate is treated with active charcoal (10 g, 283.90 mmol), and the reaction is stirred at 50° C. until peroxide is no longer detected (about 3 hours). The mixture is filtered, and the filter cake is washed by water (3*50 mL). The filtrate is treated with MeOH (2 L) and the mixture is stirred for 16 h at 4° C. The mixture is filtered, and the filter cake is washed by MeOH (3*30 mL) to obtain N3T12-029-01-2 (25 g, 142.72 mmol, 50.27%) as a white solid. ¹H NMR (400 MHz, D₂O) δ ppm 3.98 (d, J=2.2 Hz, 1H), 3.90 (ddd, J=7.4, 5.0, 2.2 Hz, 1H), 3.61 (dd, J=11.4, 5.0 Hz, 1H), 3.54 (dd, J=11.4, 7.6 Hz, 1H).

N3T12-029-01-3: To a solution of N3T12-029-01-2 (10.8 g, 34.81 mmol) in dry acetonitrile (100 mL) is added ethanedioic acid (3.13 g, 34.81 mmol) and pTSA-H₂O (0.13 g, 0.70 mmol). The reaction is stirred for 3 h at 90° C. under N₂ atmosphere. The mixture is filtered, and the filter cake is washed by acetonitrile (3*50 mL). The filtrate is concentrated in vacuo to obtain N3T12-029-01-3 (3.9 g, 33.03 mmol, 47.45%) as a colorless syrup. ¹H NMR (400 MHz, MeOD) δ 4.42 (dd, J=8.8, 6.6 Hz, 1H), 4.29 (dd, J=14.0, 7.0 Hz, 1H), 4.20 (d, J=7.2 Hz, 1H), 3.94 (dd, J=8.8, 7.2 Hz, 1H).

N3T12-029-01-4: To a solution of N3T12-029-01-3 (3.9 g, 33.03 mmol) and imidazole (4.50 g, 66.05 mmol) in ACN (50 mL) is added chlorodimethyl(2-methylprop-2-yl) silane (5.97 g, 39.63 mmol) at 0° C. dropwise under N₂ atmosphere. The reaction mixture is slowly warmed to room temperature and stirred overnight. The reaction is quenched by H₂O (20 mL) and the mixture is extracted with H₂O/DCM (3*50 mL). The combined organic layer is dried over Na₂SO₄, and the solvent is removed in vacuo. The residue is purified by flash column chromatography (eluting with 0%-20% EA in PE) to obtain N3T12-029-01-4 (3.3 g, 14.20 mmol, 43.01%) as a white solid. ¹H NMR (400 MHz, DMSO) δ 5.82 (d, J=5.2 Hz, 1H), 4.34-4.30 (dd, J=8.0, 6.4 Hz, 2H), 4.17 (ddd, J=13.8, 7.2, 5.2 Hz, 1H), 3.87 (dd, J=8.6, 7.4 Hz, 1H), 0.89 (s, 9H), 0.12 (s, 6H).

N3T12-029-01-5: To a solution of N3T12-029-01-4 (3.3 g, 14.20 mmol) in pyrdine (35 mL) is added benzoyl chloride (2.60 g, 18.46 mmol) at 0° C. The reaction is stirred for 16 h at RT under N2 atmosphere. The reaction is quenched by H₂O (30 mL), and the mixture is extracted with DCM (3*50 mL). The combined organic layer is dried over Na₂SO₄ and the solvent is removed in vacuo (evaporated with toluene three times). The residue is purified by flash column chromatography (eluting with 0%-10% EA in PE) to obtain N3T12-029-01-5 (4.65 g, 13.82 mmol, 97.31%) as colorless oil. ¹H NMR (400 MHz, DMSO) δ ppm 8.03-7.97 (m, 2H), 7.74-7.68 (m, 1H), 7.60-7.54 (m, 2H), 5.49 (q, J=7.0 Hz, 1H), 4.98 (d, J=7.0 Hz, 1H), 4.68 (dd, J=9.2, 7.2 Hz, 1H), 4.23 (dd, J=9.2, 7.0 Hz, 1H), 0.87 (s, 9H), 0.15 (s, 3H), 0.12 (s, 3H). LCMS: m/z 337.4 [(M+H)⁺].

N3T12-029-01-6: To a solution of N3T12-029-01-5 (4.65 g, 13.82 mmol) in tetrahydrofuran (40 mL) is added DIBAL-H (15.20 mL, 1 M) over 20 mins at −78° C. under N₂ atmosphere. The reaction is stirred for 5 h at −78° C. The reaction is quenched by MeOH (10 mL, added over 10 mins) at −78° C. and stirred for 20 mins at this temperature. Then the mixture is extracted with H₂O/DCM (3*40 mL), and the combined organic layer is dried over Na₂SO₄. The combined organic is concentrated in vacuo, and the residue is purified by flash column chromatography (eluting with 0%-15% EA in PE) to obtain N3T12-029-01-6 (3.87 g, 11.43 mmol, 82.73%) as a white solid. ¹H NMR (400 MHz, DMSO) δ ppm 8.01-7.94 (m, 2H), 7.72-7.64 (m, 1H), 7.59-7.50 (m, 2H), 6.54-6.28 (m, 1H), 5.25-5.02 (m, 2H), 4.34-4.15 (m, 1H), 4.16 (t, J=1.6 Hz, 1H), 3.95-3.62 (m 1H), 0.90-0.82 (m, 9H), 0.13-0.06 (m, 6H). LCMS: m/z 321.4 [(M−OH)⁺].

N3T12-029-01-7: To a solution of N3T12-029-01-6 (3.87 g, 11.43 mmol) in ACN (40 mL) is added imidazole (1.71 g, 25.15 mmol) and chlorodimethyl(2-methylprop-2-yl) silane (2.58 g, 17.15 mmol) at 0° C. under N₂ atmosphere. The reaction is stirred for 16 h at RT. The reaction is quenched by H₂O (30 mL) and extracted with H₂O/DCM (3*50 mL). The combined organic layer is dried over Na₂SO₄, and the solvent is removed in vacuo to obtain N3T12-029-01-7 (5 g, crude) as a colorless oil. The crude product is used for the next step without further purification. LCMS: m/z 321.4 [(M-OTBDMS)⁺].

N3T12-029-01-8: N3T12-029-01-7 (5 g, 11.04 mmol) is dissolved in MeOH saturated with ammonia (50 mL, 350.00 mmol, 7 M). The reaction is stirred for 48 h at RT. The solvent is removed in vacuo, and the residue is purified by flash column chromatography (eluting with 0%-15% EA in PE) to obtain N3T12-029-01-8 (3.3 g, 9.47 mmol, 85.71%) as a colorless oil (two isomers). ¹H NMR (400 MHz, DMSO) δ ppm 5.10 (d, J=4.8 Hz, 1H), 5.00 (d, J=1.4 Hz, 1H), 3.96 (dd, J=8.6, 6.0 Hz, 1H), 3.91-3.86 (m, 2H), 3.62 (dd, J=8.4, 5.6 Hz, 1H), 0.87 (s, 18H), 0.07 (dd, J=4.4, 2.0 Hz, 12H). 1H NMR (400 MHz, DMSO) δ ppm 5.13 (t, J=4.8 Hz, 2H), 4.07-4.01 (m, 1H), 4.06-4.01 (m, 1H), 3.81 (dd, J=5.2, 3.8 Hz, 1H), 3.41 (dd, J=8.4, 4.4 Hz, 1H), 0.87 (d, J=3.8 Hz, 18H), 0.08-0.05 (m, 12H).

N3T12-029-01-9: To a solution of N3T12-029-01-8 (3.1 g, 8.89 mmol) in tetrahydrofuran (15 mL) is added LiHMDS (19.56 mL, 1 M) at −78° C. The reaction is stirred for 1 hour at this temperature under a N₂ atmosphere. Then (diethoxyphosphoryl)methyl trifluoromethanesulfonate (6.67 g, 22.23 mmol) is added dropwise, and the reaction is warmed up to 0° C. slowly. The reaction is stirred for 2 hours at 0° C. The solvent is quenched by H₂O (30 mL) and extracted with H₂O/DCM (3*50 mL). The combined organic layer is dried over Na₂SO₄, and the solvent is removed in vacuo. The residue is purified by flash column chromatography (eluting with 0%-60% EA in PE) to obtain N3T12-029-01-9 (3.35 g, 6.72 mmol, 75.54%) as a colorless oil. ¹H NMR (400 MHz, DMSO) δ ppm 5.15-5.02 (m, 1H), 4.12-3.88 (m, 7H), 3.84 (dd, J=9.0, 6.2 Hz, 2H), 3.80-3.58 (m, 1H), 1.23 (t, J=7.0 Hz, 6H), 0.87-0.86 (m, 18H), 0.13-0.02 (m, 12H). ³¹P NMR (162 MHz, DMSO) δ ppm 20.78, 20.53.

N3T12-029-01-10: To a solution of N3T12-029-01-9 (1.85 g, 3.71 mmol) in water (3 mL) is added TFA (9 mL) at 0° C. The reaction is stirred for 2 h at RT. TLC shows the reaction is complete. The reaction mixture is concentrated in vacuo, and the residue is purified by flash column chromatography (eluting with 0% to 10% MeOH in DCM) to obtain N3T12-029-01-10 (798 mg, 2.95 mmol, 79.80%) as a colorless oil. ¹H NMR (400 MHz, DMSO) δ ppm 6.18-6.14 (t, J=7.8 Hz, 1H), 5.46-5.07 (m, 1H), 5.05-4.89 (m, 1H), 4.12-3.88 (m, 6H), 3.88-3.69 (m, 4H), 1.23 (t, J=7.0 Hz, 6H). ³¹P NMR (162 MHz, DMSO) δ ppm 21.07, 21.04.

N3T12-029-01-11: To a solution of N3T12-029-01-10 (798 mg, 2.95 mmol) in Pyrdine (10 mL) is added benzoyl chloride (1.25 g, 8.86 mmol) at 0° C. The reaction is warmed up to RT and stirred for 16 hours under a N₂ atmosphere. The reaction is quenched by H₂O (20 mL), and the mixture is extracted with H₂O/DCM (3*20 mL). The combined organic layer is dried over Na₂SO₄, and the solvent is removed in vacuo (evaporated with toluene three times). The residue is purified by flash column chromatography (eluting with 0%-95% EA in PE) to obtain N3T12-029-01-11 (950 mg, 1.99 mmol, 67.24%) as a colorless oil. LCMS: m/z 501.3 [(M+Na)⁺].

N3T12-029-01-12: To a solution of 1,2,3,4-tetrahydropyrimidine-2,4-dione (235 mg, 2.10 mmol) is added N,O-Bis(trimethylsilyl) acetamide (940 mg, 4.62 mmol). The reaction is stirred for 10 mins at 65° C. under a N₂ atmosphere. Then, the reaction mixture is cooled down to 0° C. and N3T12-029-01-11 (670 mg, 1.40 mmol), SnCl₄ (1.46 g, 5.60 mmol) is added dropwise under ice bath. The reaction is stirred for 4 hours at RT. The reaction is quenched by H₂O (3 mL), and the solvent is removed in vacuo (evaporated with THE three times). The residue is purified by flash column chromatography (eluting with 0% to 7% MeOH in DCM) to obtain N3T12-029-01-12 (650 mg, 1.39 mmol, 99.09%) as a white solid. ¹H NMR (400 MHz, DMSO) δ ppm 11.42 (s, 1H), 8.03 (d, J=7.4 Hz, 2H), 7.72 (t, J=7.4 Hz, 1H), 7.65-7.54 (m, 3H), 6.03 (d, J=1.4 Hz, 1H), 5.62 (dd, J=8.0, 2.0 Hz, 1H), 5.43 (s, 1H), 4.40-4.34 (m, 2H), 4.16 (dd, J=10.8, 4.2 Hz, 1H), 4.09-4.00 (m, 6H), 1.23 (t, J=7.0 Hz, 6H). 31P NMR (162 MHz, DMSO) δ ppm 20.54. LCMS: m/z 469.3 [(M+H)⁺].

N3T12-029-01-13: N3T12-029-01-12 (650 mg, 1.39 mmol) is dissolved in MeOH with saturated ammonia (5 mL, 35.00 mmol, 7 M). The reaction is stirred for 16 h at RT. The solvent is removed in vacuo, and the residue is purified by flash column chromatography (eluting with 0%-10% MeOH in DCM) to obtain N3T12-029-01-13 (467 mg, 1.28 mmol, 92.38%) as a white solid. ¹H NMR (400 MHz, DMSO) δ ppm 11.32 (s, 1H), 7.53 (d, J=8.0 Hz, 1H), 5.94 (d, J=4.0 Hz, 1H), 5.66 (d, J=1.0 Hz, 1H), 5.51 (dd, J=8.0, 2.0 Hz, 1H), 4.27 (d, J=10.2 Hz, 1H), 4.16 (s, 1H), 4.07-3.97 (m, 6H), 3.95-3.83 (m, 2H), 1.22 (td, J=7.0, 1.2 Hz, 6H). LCMS: m/z 365.1 [(M+H)⁺].

N3T12-029-01: To a solution of N3T12-029-01-13 (467 mg, 1.28 mmol) in dry DCM (10 mL) is added diisopropylammonium tetrazolide (329 mg, 1.92 mmol) and 3-{[2,6-dimethyl-3,5-di(prop-2-yl)-3,5-diaza-4-phosphahept-4-yl]oxy}propanenitrile (773 mg, 2.56 mmol) at 0° C. under N2 atmosphere. The reaction is stirred for 16 h at RT. The reaction is extracted with NaHCO₃(aq)/DCM (3*20 mL), and the combined organic layer is dried over Na₂SO₄. The solvent is removed in vacuo, and the residue is purified by reverse phase column (eluting with 0%-60% ACN in H₂O) to obtain N3T12-029-01 (585 mg, 1.04 mmol, 80.84%) (another 150 mg is obtained from small scale reaction) as a colorless syrup. LCMS: m/z 565.2 [(M+H)⁺]. ¹H NMR (400 MHz, DMSO) δ ppm 11.36 (s, 1H), 7.55 (dd, J=8.0, 1.6 Hz, 1H), 5.77 (d, J=19.6 Hz, 1H), 5.54 (dd, J=8.0, 1.8 Hz, 1H), 4.45-4.32 (m, 2H), 4.21-4.12 (m, 1H), 4.06-3.93 (m, 6H), 3.91-3.71 (m, 3H), 3.64-3.52 (m, 2H), 2.81-2.75 (m, 2H), 1.23-1.19 (m, 6H), 1.16-1.12 (m, 12H). ³¹P NMR (162 MHz, DMSO) δ ppm 150.04, 149.54, 20.52, 20.48.

The effect of incorporating N3T12-029-01 or another nucleotide analogue, VPU, at the 5′-terminus of the antisense strand is assessed.

TABLE 33
VEGFA siRNAs with RISC Loading Modifications at Position 1 of the Antisense
Strand.

| | SEQ ID | | | |
|---|---|---|---|---|
| Duplex | NO | sense strand (5′ to 3′) | SEQ ID NO | antisense strand (5′ to 3′) |
| N3T120034-01 | 274 | AGAGAAAAGAGAAAGUGU | 275 | [N3T12-029- |
| | | U | | 01]ACACUUUCUCUUUUCU |
| | | | | CUUU |
| N3T120035-01 | 276 | UGAAUGCAGACCAAAGAA | 277 | [N3T12-029- |
| | | AGA | | 01]CUUUCUUUGGUCUGCA |
| | | | | UUCACA |
| N3T120036-01 | 278 | AGAGAAAAGAGAAAGUGU | 279 | [VPU]ACACUUUCUCUUUU |
| | | U | | CUCUUU |
| N3T120037-01 | 280 | UGAAUGCAGACCAAAGAA | 281 | [VPU]CUUUCUUUGGUCUG |
| | | AGA | | CAUUCACA |
| N3T120038-01 | 282 | AGAGAAAAGAGAAAGUGU | 283 | AACACUUUCUCUUUUCUC |
| (unmodified | | U | | UUU |
| control) | | | | |
| N3T070162-01 | 284 | UGAAUGCAGACCAAAGAA | 285 | UCUUUCUUUGGUCUGCAU |
| (unmodified | | AGA | | UCACA |
| control) | | | | |

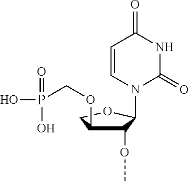

[N3T12-029-01] 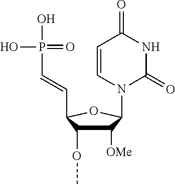
[VPU]

Dose Screens of VEGFA dsRNA Duplexes with RISC Loading Modifications at Position 1 of the Antisense Strand in HuH7

The siRNAs (Table 33) are transfected into Human Hepatocyte-derived cell line HuH7 with RNAiMAX (Invitrogen, 13778075) in a 96-well plate. All the siRNAs are tested at 10 nM and 1 nM (Table 34). Twenty-four hours after transfection, the culture medium is discarded, and cells are collected for RNA extraction. Total RNA is extracted by RNeasy® 96 Kit (QIAGEN-74182) according to the kit instructions, and reverse transcribed to cDNA by FastKing RT Kit (with gDNase) (Tiangen-KR116-02). The cDNA of the target gene is quantified by qPCR with specific primer and probe sets. B-actin cDNA is measured as housekeeping gene in parallel. 8 L PCR mixture and 2 μL sample cDNA are added to 384-well PCR plates. The PCR is performed with 95° C. for 10 minutes, then cycling at 95° C. 15 seconds and 60° C. 1 minute for 40 cycles.

TABLE 34
Dose Screens of VEGFA siRNAs with RISC Loading Modifications
at Position 1 of the Antisense Strand in HuH7 Cells

| | 10 nM Dose | | 1 nM Dose | |
|---|---|---|---|---|
| | Average % | | Average % | |
| | VEGFA mRNA | | VEGFA mRNA | |
| Duplex | Remaining | ST DEV | Remaining | STDEV |
| N3T120034-01 | 24.79 | 0.02 | 23.24 | 0.62 |
| N3T120035-01 | 30.79 | 0.42 | 32.59 | 0.67 |
| N3T120036-01 | 25.1 | 1.97 | 22.52 | 1.67 |
| N3T120037-01 | 20.4 | 0.32 | 20.55 | 0.25 |
| N3T120038-01 | 17.72 | 1.87 | 21.7 | 0.16 |
| N3T070162-01 | 16.57 | 0.85 | 18.24 | 1.02 |

The guide strand of a siRNA duplex may bear a 5′-phosphate or other analogs to bind the effector protein of the RNA-induced silencing complex Argonaute 2 (AGO2). There are no modifications on N3T120038-01 and N3T070162-01, which have good VEGFA mRNA inhibition. N3T120034-01, N3T120035-01, N3T120036-01, and N3T120037-01 have relatively high knock down efficacy at 10 nM and 1 nM.

1. A RNAi agent comprising a sense sequence and a complementary antisense sequence, wherein the RNAi agent comprises one or more sequences corresponding to a region of a LRRK2 gene, wherein the antisense sequence is comprises a sequence selected from SEQ ID NO. 46, 70, 192, 194, 138, 56, and 68 and/or the sense sequence comprises a sequence selected from SEQ ID NO: 45, 69, 191, 193, 137, 55, and 67.

2. (canceled)

3. (canceled)

4. The RNAi agent of claim 1, wherein the RNAi agent is conjugated to a lipophilic moiety.

5. The RNAi agent of claim 1, wherein the siRNA or ASO comprises an off-target modification, wherein the modification is effective in reducing off-target effects and is at one or both ends of the sense or antisense strand or within any position of the sense or antisense strand.

6. The RNAi agent of claim 1, wherein the sense and antisense nucleotide sequences have a length of 15-30 nucleotides, wherein one strand is 1, 2, 3, or more nucleotides longer than the other, so that there is an overhang at one or both ends; wherein the sense strand is 19 nucleotides in length and the antisense strand is 21 nucleotides in length, or wherein the sense strand is 20 nucleotides in length and the antisense strand is 22 nucleotides in length, or wherein the sense strand is 21 nucleotides in length and the antisense strand is 23 nucleotides in length.

7. The RNAi agent of claim 1, wherein the sense and antisense nucleotide sequences have a length of 15-30 nucleotides, wherein the sequences have a blunt end on one or both ends of the sense sequence and the complementary antisense sequence.

8. (canceled)

9. (canceled)

10. The RNAi agent of claim 1, (i) comprising a sense or antisense sequence selected from SEQ ID NO: 221, 46, 224, 192, 237, and 194; or (ii) comprising a duplex selected from SEQ ID NO: 221 and 46; SEQ ID NO: 224 and 192; SEQ ID NO: 234 and 70; and SEQ ID NO: 237 and 194.

11. (canceled)

12. The RNAi agent of claim 1, wherein the RNAi agent comprises one or more chemical modifications selected from a phosphorylated 5′ end, a 3′ end overhang, a phosphonated 5′ end, a UNA and/or GNA motif, a lipophilic moiety, a phosphorothioate linkage, a modified nucleotide such as 2′-deoxy-2′-fluoro and/or 2′-O-methyl ribosugar, and combinations thereof; and wherein the siRNA duplex is selected from the duplexes of SEQ ID NOs 288 and 289; SEQ ID NOs 292 and 293; SEQ ID NOs 296 and 297; and SEQ ID NOs 298 and 299.

13. (canceled)

14. (canceled)

15. (canceled)

16. The RNAi agent of claim 1, wherein the seed region nucleotides (nucleotides 2-8) on the antisense strand may include one or more of the following nucleosides: 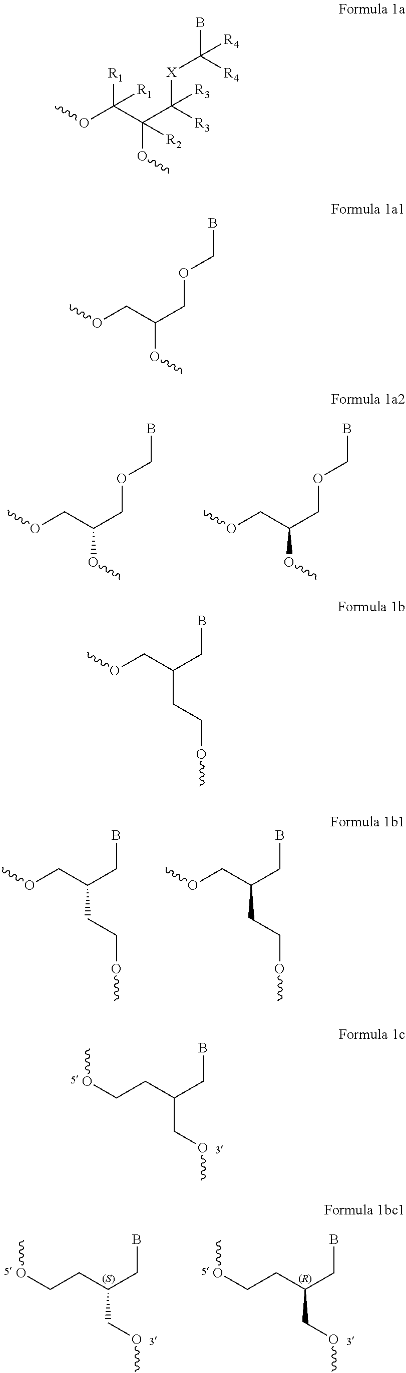
wherein
X═O, S, NH, —CONH—, or —NHCO—; and
B is a modified or unmodified nucleobase, R1, R2, R3 and R4 are independently are H, halogen, OR5, or alkyl; and R5 is H, alkyl, cycloalkyl, aryl, aralkyl, heteroaryl or sugar.

17. The RNAi agent of claim 1, wherein the RNAi agent is an RNA duplex comprising a sense strand and an antisense strand to form a siRNA, wherein the sense strand comprises one or more lipophilic moieties selected from the following:
lipophilic moieties L₁ or L₂ conjugated to the siRNA via a phosphodiester or phosphorothioate bond accordance with Formula 2: 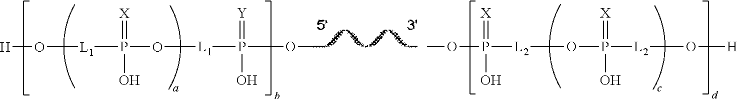
where ein
X═O or S;
a=0-4;
b=0-4;
c=0-4;
d=0-4;
provided that b and d are not both 0; and the ribbon represents the sense strand, wherein 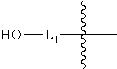
of the sense strand is in accordance with one of the following formulae: 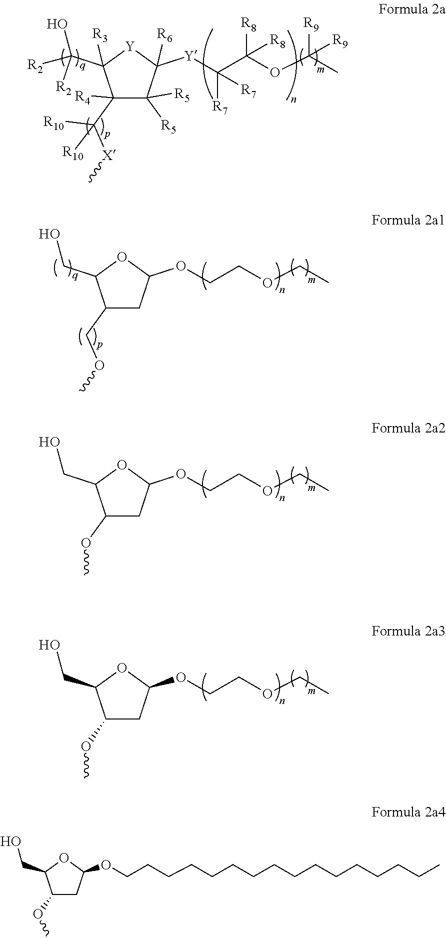
wherein,
R₂, R₃, R₄, R₅, R₆, R₇, R₈, R₉, R₁₀ are each independently H, halogen, or C1-C6 alkyl, optionally substituted with one or more halogen and/or hydroxy:
X′ is O, S, or NH;
Y is O, S, NH or CH₂;
Y′ is O, S, CH₂, NH, —NHCO—, —CONH—, or —SS—;
p=0-10, preferably 0-2;
q=0-10, preferably 1-2;
n=0-10;
m=1-30; 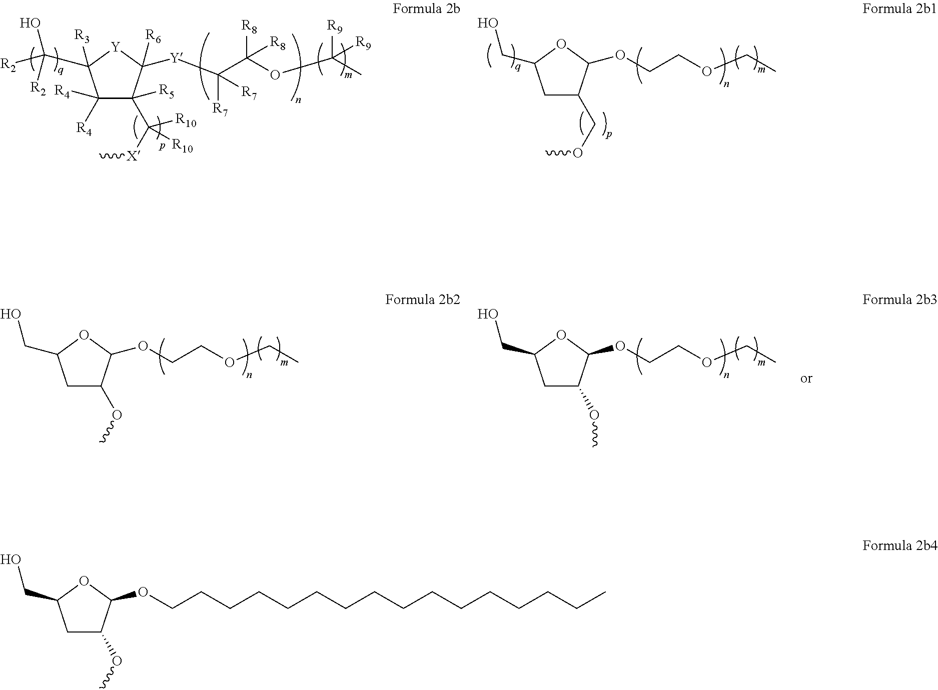
wherein,
R₂, R₃, R₄, R₅, R₆, R₇, R₈, R₉, R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, preferably 0-2;
q=0-10, preferably 1-2;
n=0-10;
m=1-30;
or wherein 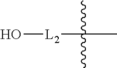
of the sense strand is in accordance with one of the following formulae: 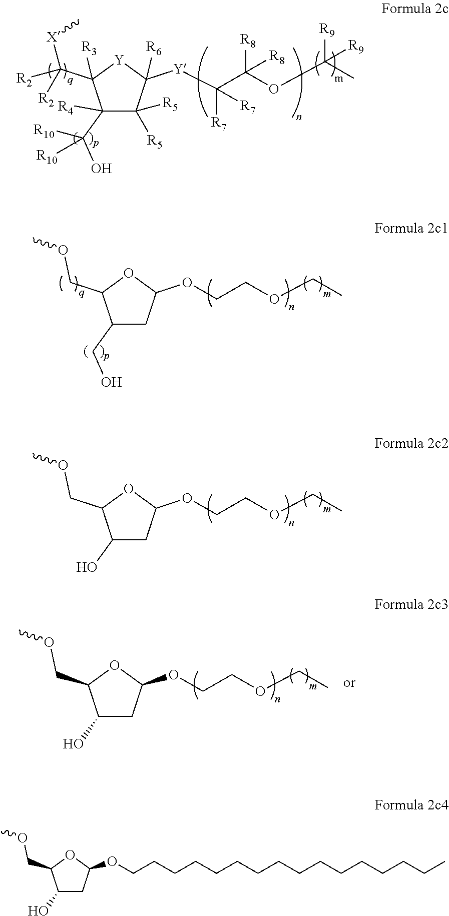
wherein,
R₂, R₃, R₄, R₅, R₆, R₇, R₈, R₉, R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, preferably 0-2;
q=0-10, preferably 1-2;
n=0-10;
m=1-30; 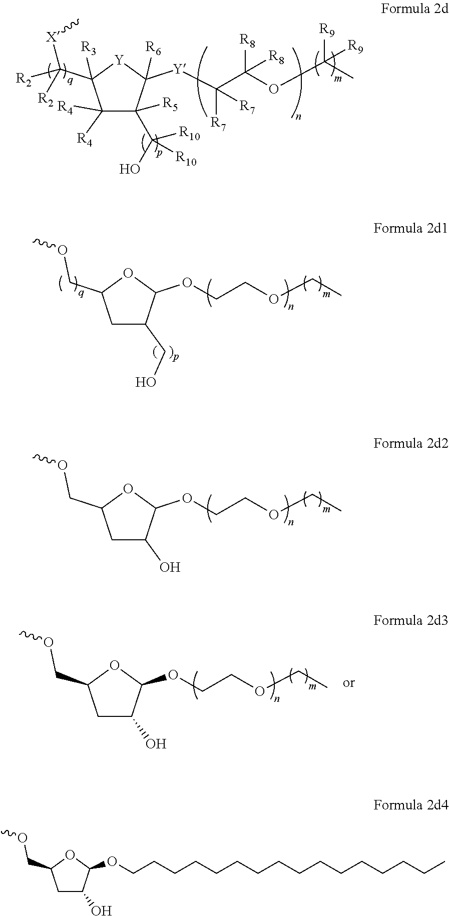
wherein,
R₂, R₃, R₄, R₅, R₆, R₇, R₈, R₉, R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, preferably 0-2;
q=1-10, preferably 1-2;
n=0-10;
m=1-30;
or wherein 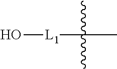
of the sense strand is in accordance with one of the following formulae: 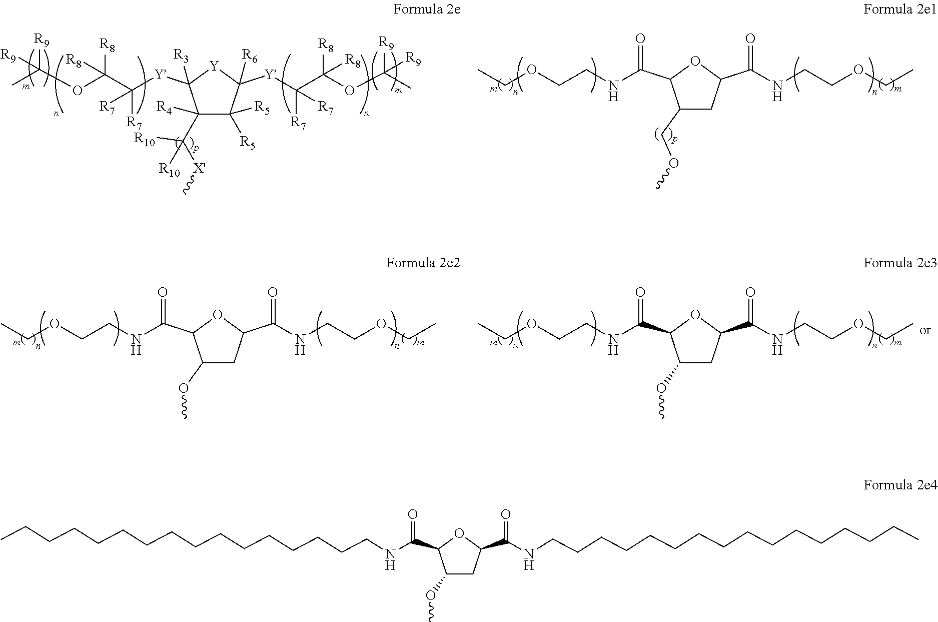
wherein,
R₃, R₄, R₅, R₆, R₇, R₈, R₉, and R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, preferably 0-2;
n=0-10;
m=1-30; 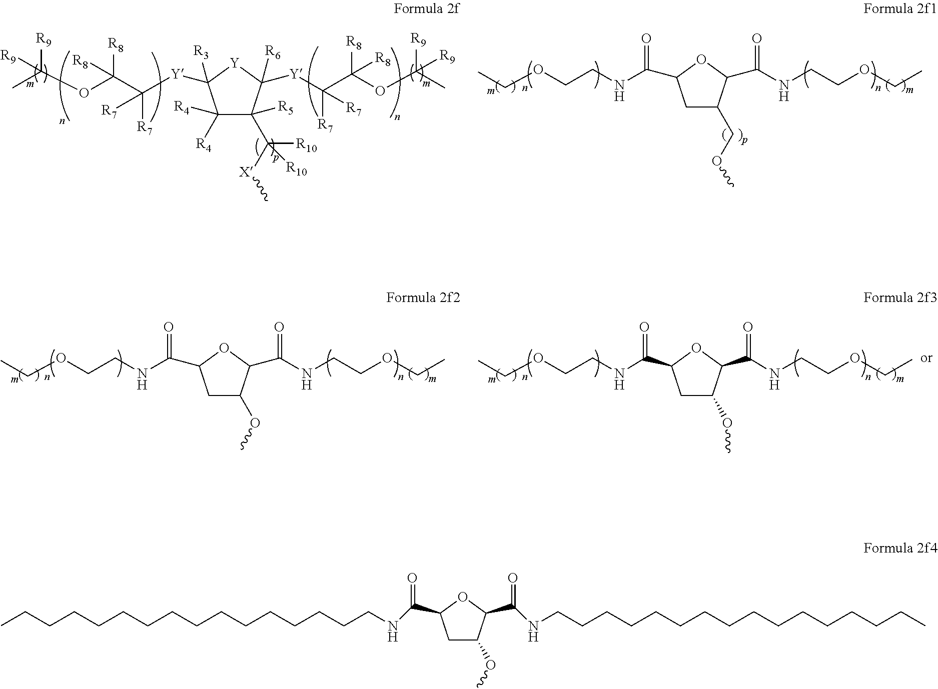
wherein,
R₃, R₄, R₅, R₆, R₇, R₈, R₉, and R₁₀ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, or —SS—;
p=0-10, preferably 0-2;
q=0-10;
n=0-10;
m=1-30;
or wherein 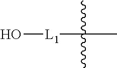
of the sense strand is in accordance with one of the following formulae: 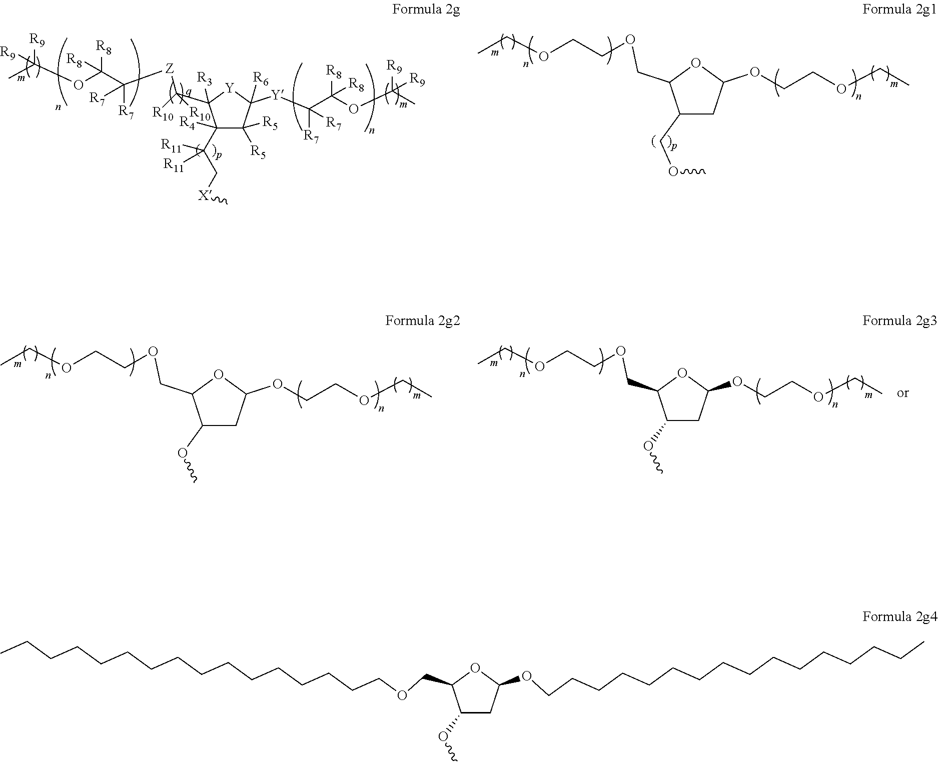
wherein,
R₂, R₃, R₄, R₅, R₆, R₇, R₈, R₉, R₁₀ and Run are each independently H, halogen, or C1-C6 alkyl, optionally substituted with one or more halogen and/or hydroxy:
X′ is O, S, or NH;
Y is O, S, NH or CH₂;
Y′ is O, S, CH₂, NH, —NHCO—, —CONH—, or —SS—;
p=0-10, preferably 0-2;
g=0-10, preferably 1-2;
n=0-10;
m=1-30;
or 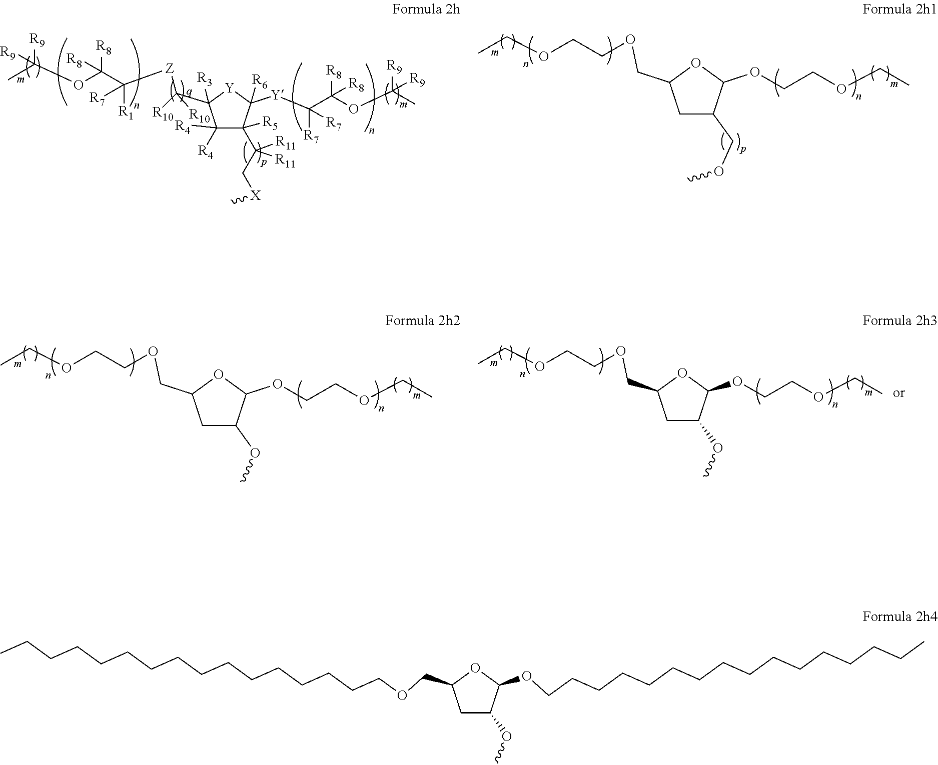
wherein,
R₃, R₄, R₅, R₆, R₇, R₈, R₉, R₁₀ and R₁₁ are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, —CH₂O—, —OCH₂—, or —SS—;
Z is O, S, CH₂, NH, —NHCO—, —CONH—, or —SS—;
p=0-10, preferably 0-2;
q=1-10, preferably 1-2;
n=0-10;
m=1-30;
provided that the O with the wavy line on said L1 or L2 is part of a phosphodiester or phosphorothiate bond to the sense strand;
for example, 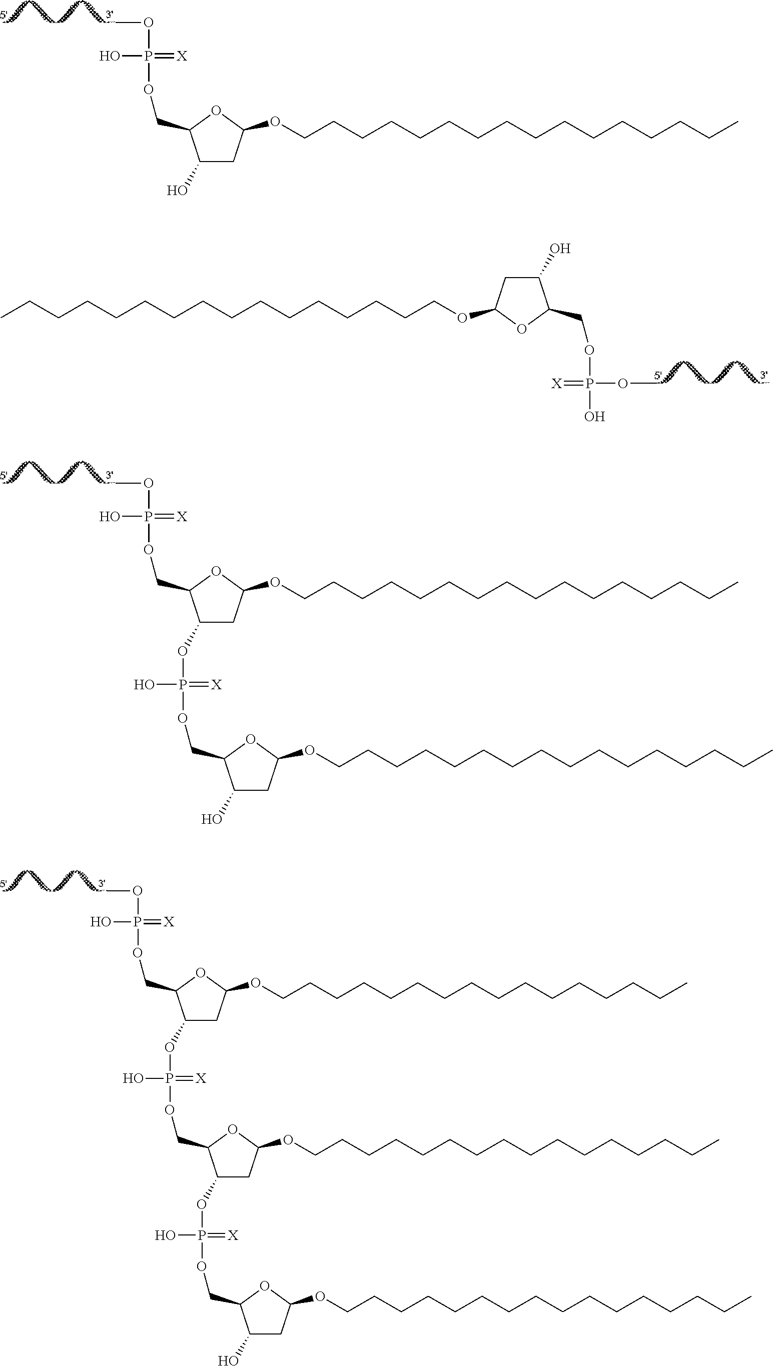 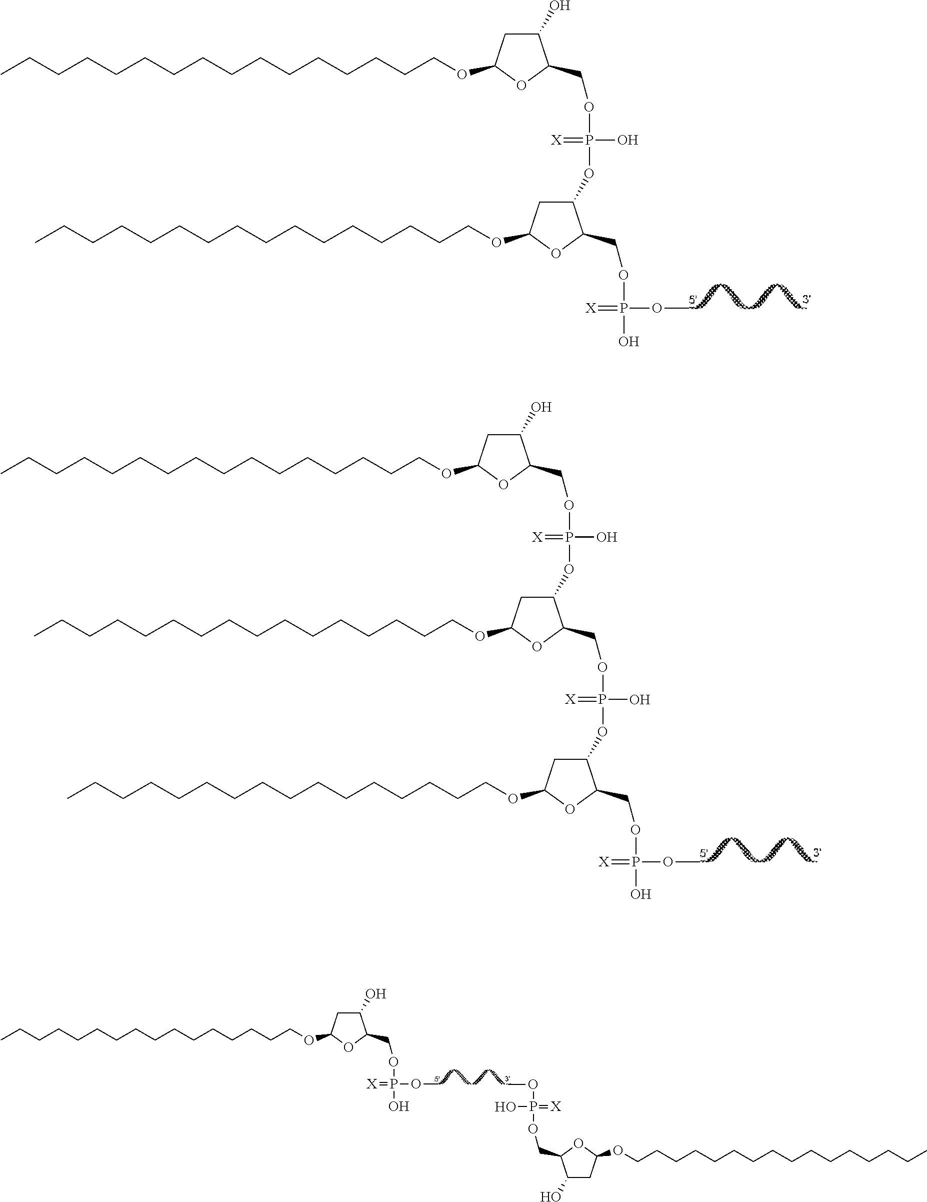 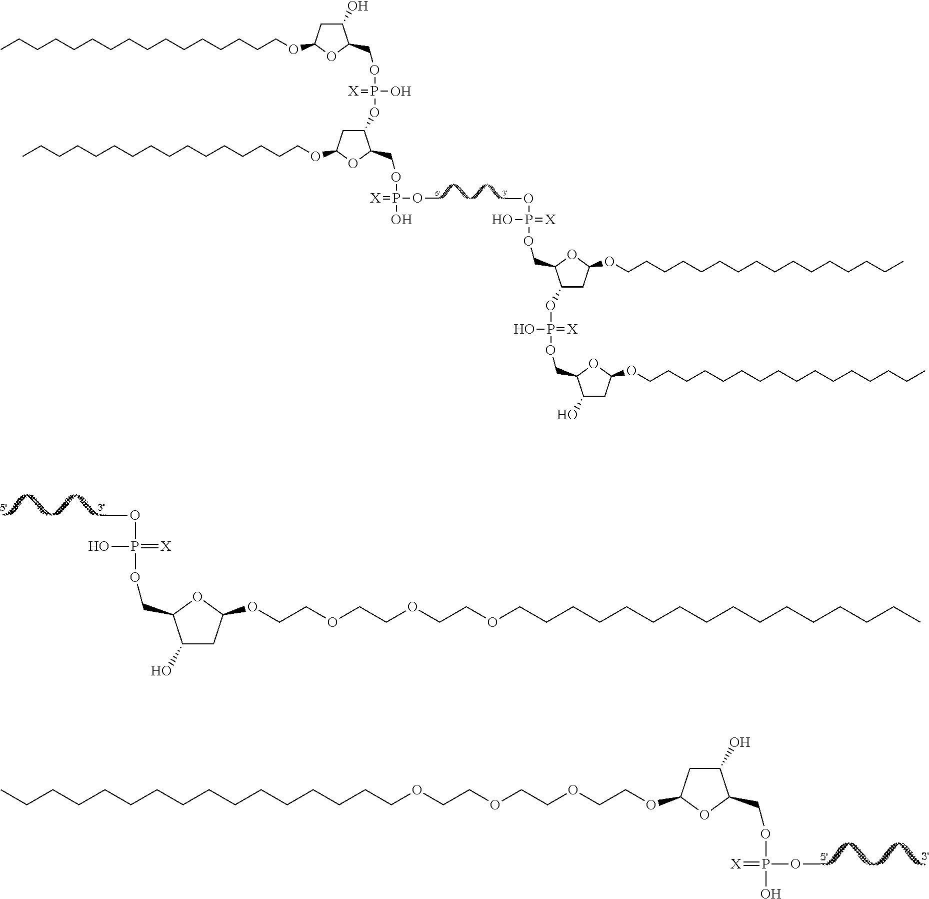 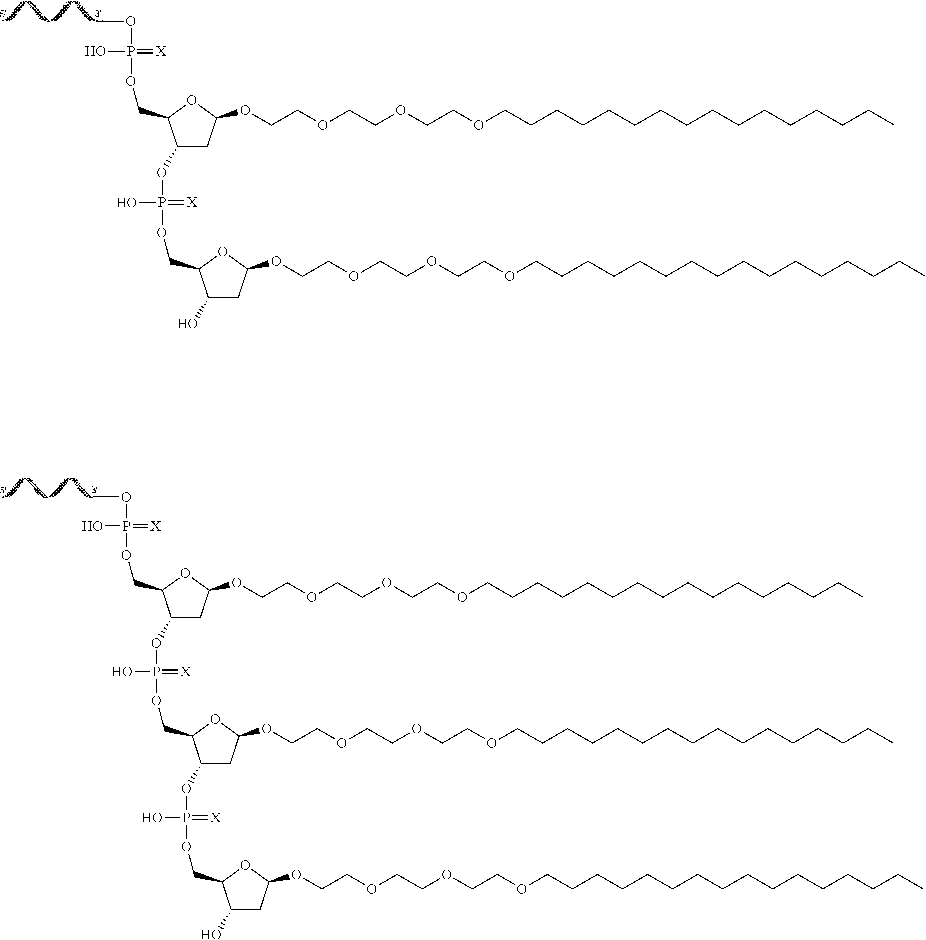 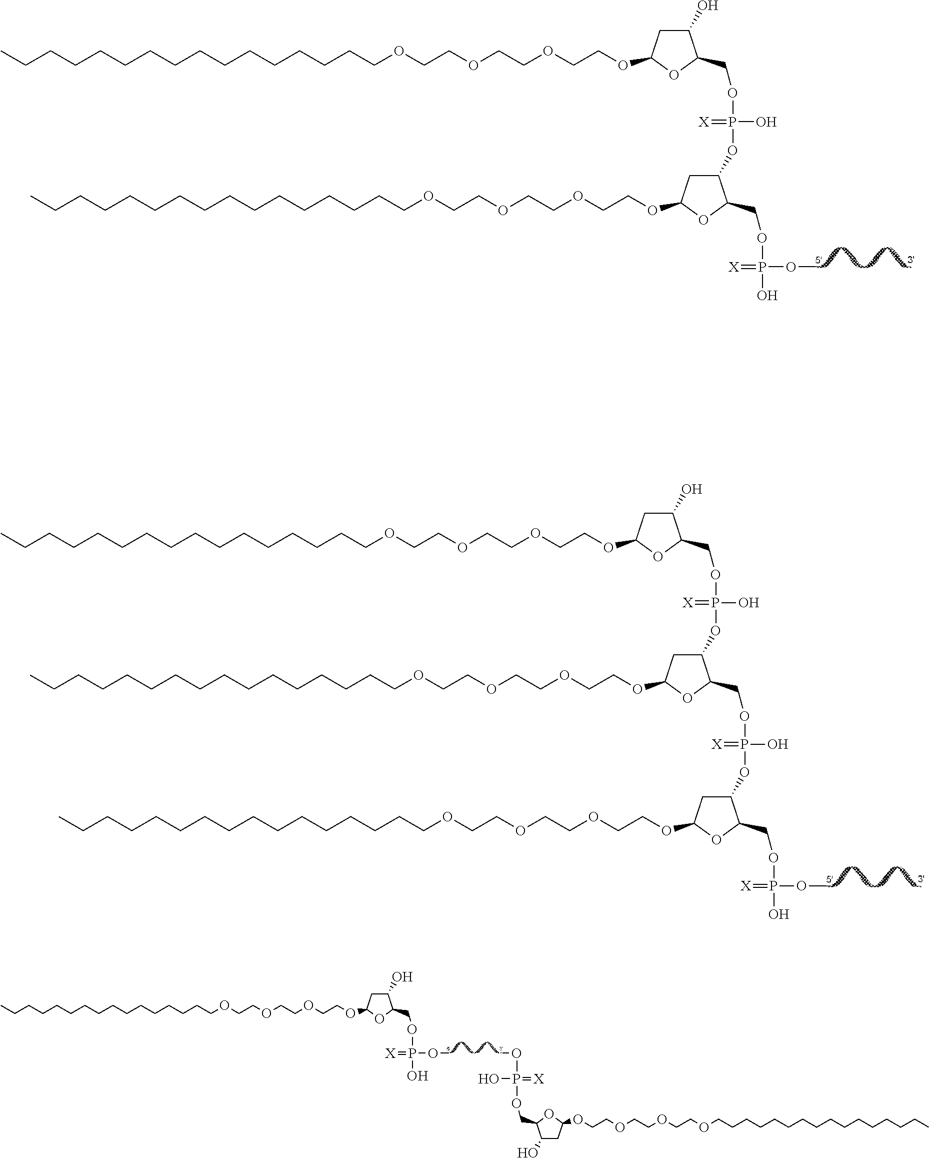 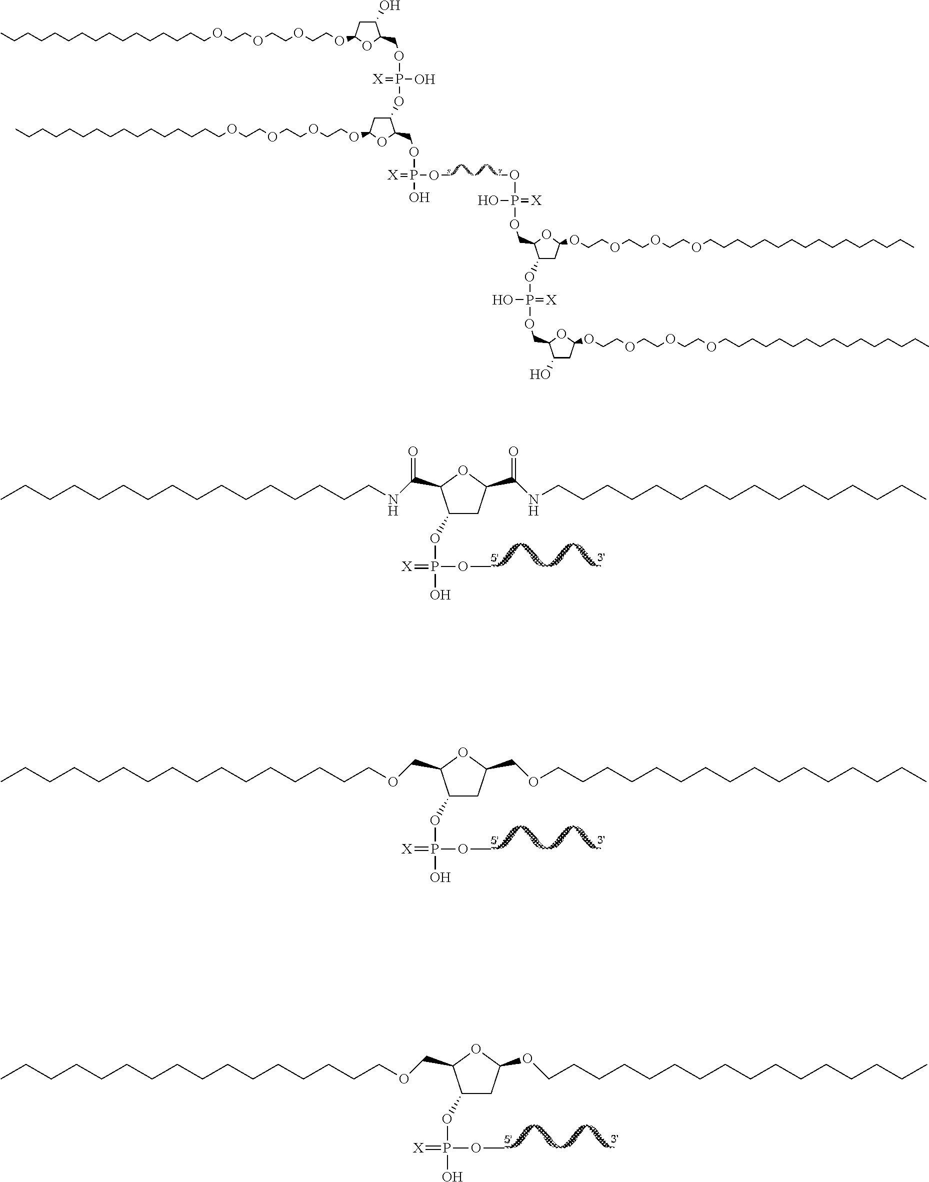
wherein
X═O or S; and the ribbon represents the sense strand,
or wherein selected nucleotides are linked to 2′-O lipid conjugate moieties or adamantyl groups on sense strand, in accordance with one of the following formulae: 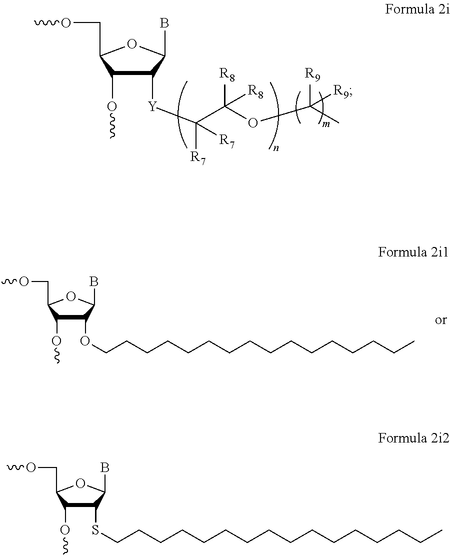
wherein,
R₇, R₈ and R₉ are each independently H, halogen, or C1-C6 alkyl, optionally substituted with one or more halogen and/or hydroxy;
Y′ is O, S, CH₂, NH, —NHCO—, —CONH—, —CH₂O—, or —SS—;
n=0-10;
m=1-30;
or wherein selected nucleotides are linked to 5′O-lipid conjugate moieties or adamantyl groups on sense strand, in accordance with one of the following formulae: 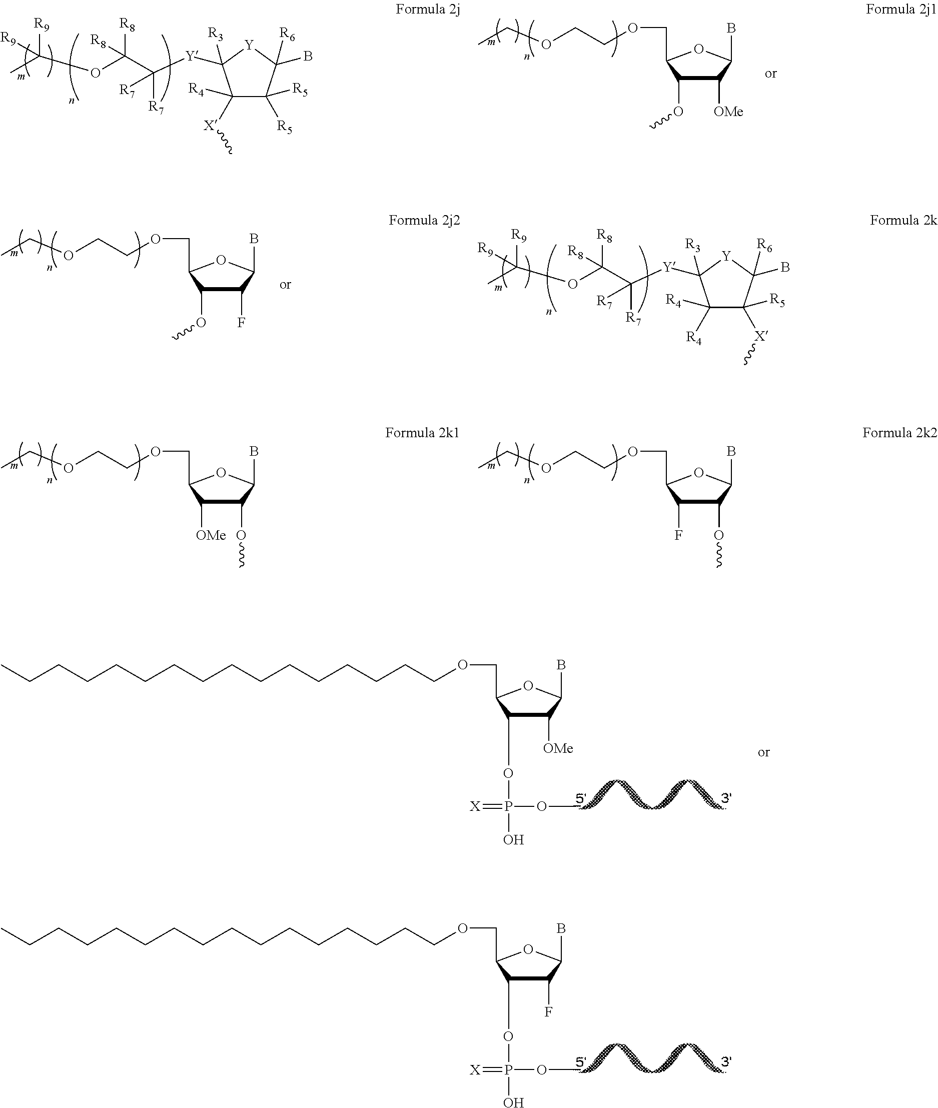
wherein B is a modified or unmodified nucleobase; and the ribbon represents the sense strand
R₃, R₄, R₅, R₆, R₇, R₈, and R_{y} are each independently H, halogen, or C1-C6 alkyl, optionally substituted with one or more halogen and/or hydroxy;
X′ is O, S, or NH;
Y is O, S, NH or CH₂;
Y′ is O, S, CH₂, NH, —NHCO—, —CONH—, —CH₂O—, —OCH₂—, or —SS—;
n=0-10;
m=1-30;
or wherein 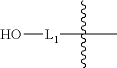
of the sense strand is in accordance with one of the following formulae: 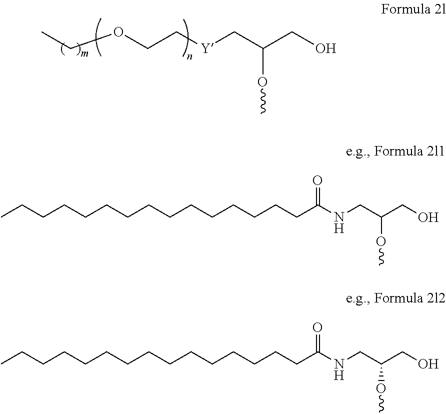
wherein,
Y′ is O, S, CH₂, NH, —NHCO—, —CONH—, or —SS—;
n=0-10;
m=1-30;
or wherein 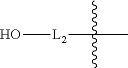
of the sense strand is in accordance with one of the following formulae: 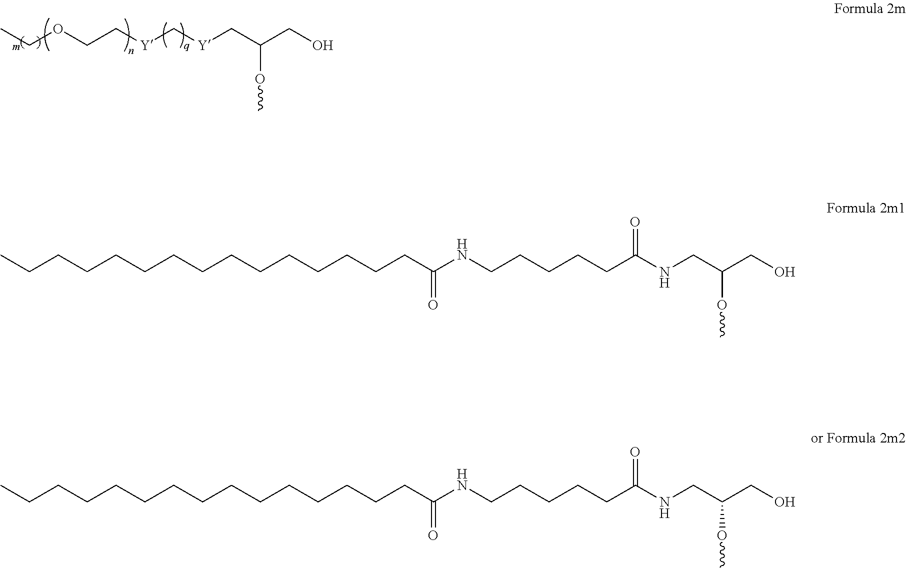
wherein,
Y′ is O, S, CH₂, NH, —NHCO—, —CONH—;
q=1-20;
n=0-10;
m=1-30;
or wherein 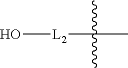
of the sense strand is in accordance with one of the following formulae: 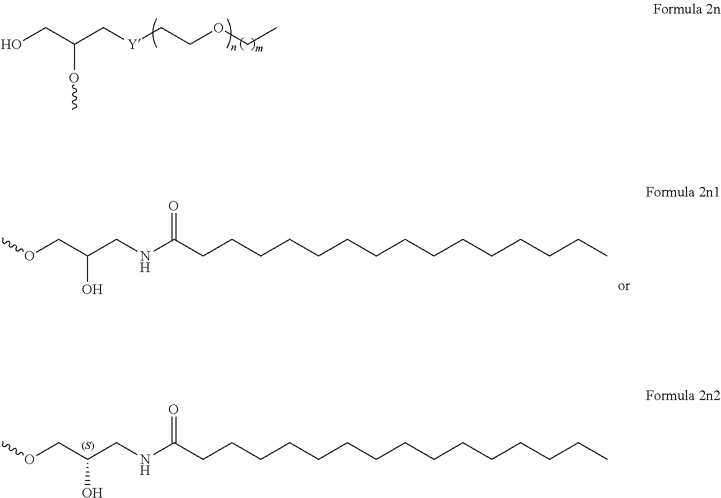
wherein,
Y′ is O, S, CH₂, NH, —NHCO—, —CONH—;
n=0-10;
m=1-30;
or wherein 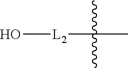
of the sense strand is in accordance with one of the following formulae: [chemical structure] 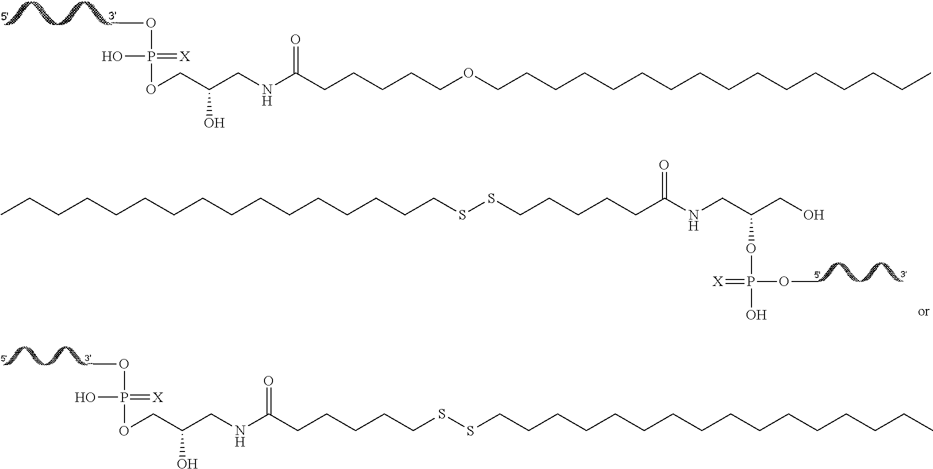
wherein
X═O or S; and the ribbon represents the sense strand,
wherein,
Y′ is O, S, CH₂, NH, —NHCO—, —CONH—, or —SS—;
q=0-10;
n=0-10;
m=1-30;
or wherein 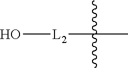
of the sense strand is in accordance with one of the following formulae: 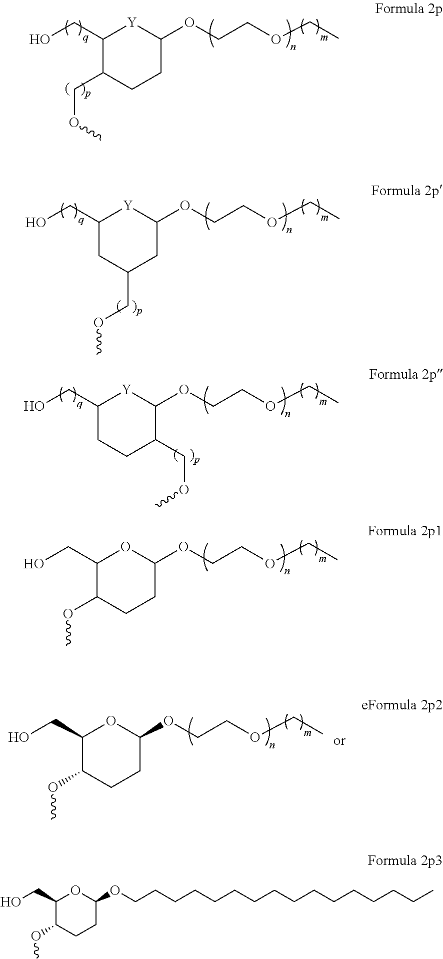
wherein,
Y is O, S, NH or CH₂;
p=0-10,
q=0-10;
n=0-10;
m=1-30;
or wherein 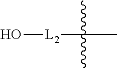
of the sense strand is in accordance with one of the following formulae: 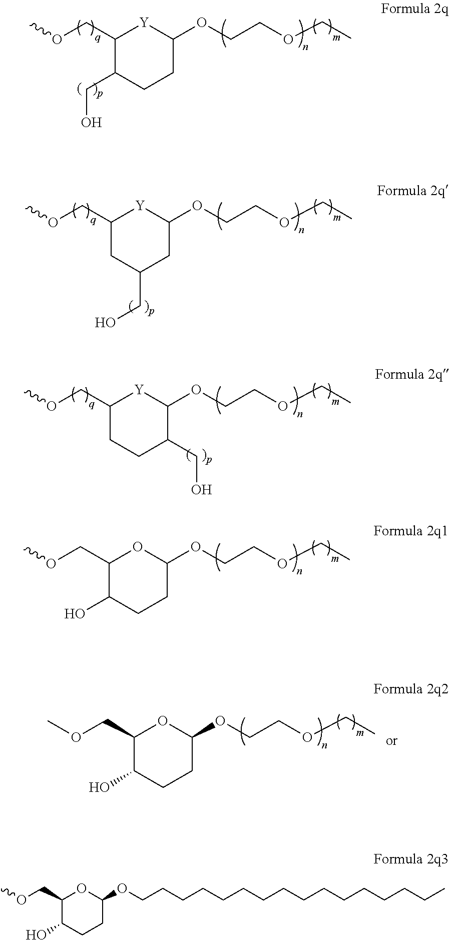
wherein,
Y is O, S, NH or CH₂;
p=0-10,
q=0-10;
n=0-10;
m=1-30;
or wherein 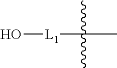
of the sense strand is in accordance with one of the following formulae: 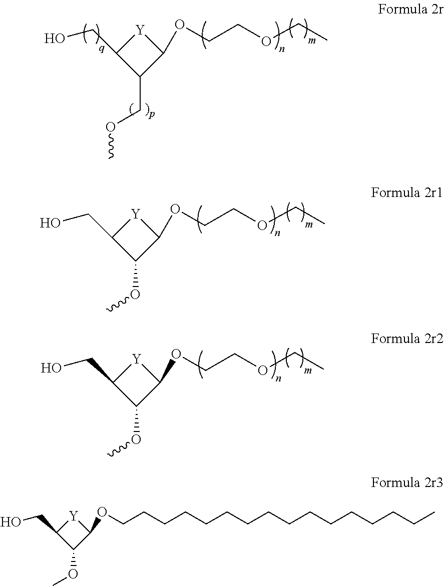
wherein,
Y is O, S, NH or CH₂;
p=0-10,
q=0-10;
n=0-10;
m=1-30;
or wherein 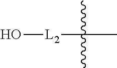
of the sense strand is in accordance with one of the following formulae: 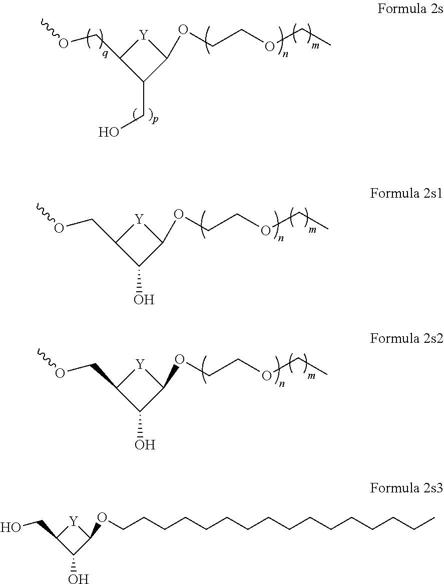
wherein,
Y is O, S, NH or CH₂;
p=0-10,
g=0-10;
n=0-10;
m=1-30;
or wherein the sense strand of the siRNA duplex is modified at one or both ends with one or more lipophilic moieties comprising one or more of the following formulae: 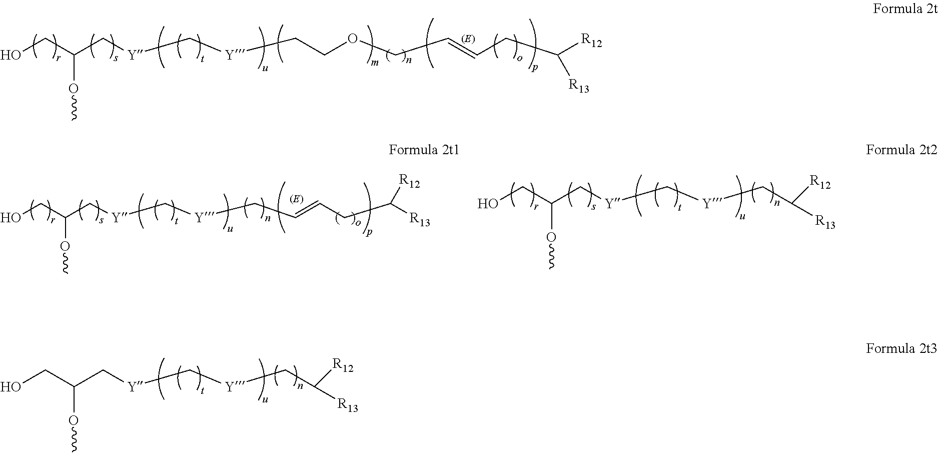
wherein,
R₁₂ and R₁₃ are each independently selected from H, halogen, and C₁-C₆ alkyl, optionally substituted with one or more halogen and/or hydroxy;
Y″ is O, S, CH₂, NH, —NHCO—, —CONH—, or —SS—;
r=1-10, preferably 1-3;
s=1-10, preferably 1-3;
t=1-10;
u=0-3;
m=0-5;
n=1-30;
o=1-5,
or wherein the sense strand of the siRNA duplex is modified at one or both ends with one or more lipophilic moieties comprising one or more of the following formulae: 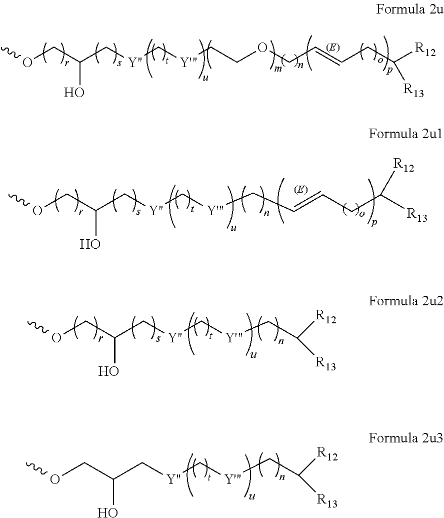
wherein,
R₁₂ and R₁₃ are each independently selected from H, halogen, and C₁-C₆ alkyl, optionally substituted with one or more halogen and/or hydroxy;
Y″ is O, S, CH₂, NH, —NHCO—, —CONH—, or —SS—;
Y′″ is O, S, CH₂, NH, —NHCO—, —CONH, or —SS—;
r=1-10, preferably 1-3;
s=1-10, preferably 1-3;
t=1-10;
u=0-3;
m=0-5;
n=1-30;
o=1-5;
or wherein the sense strand of the siRNA duplex is modified at one or both ends with one or more moieties comprising one or more of the following structures: 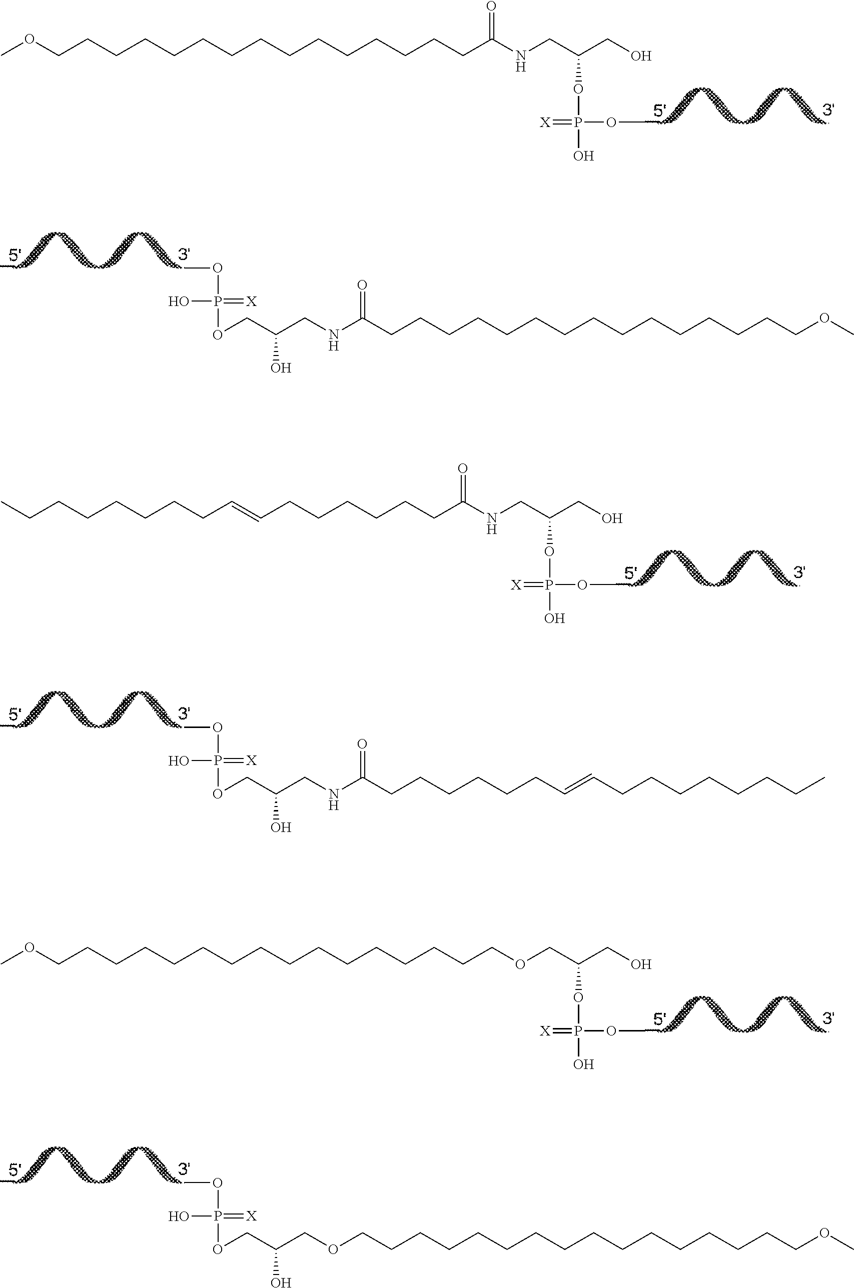 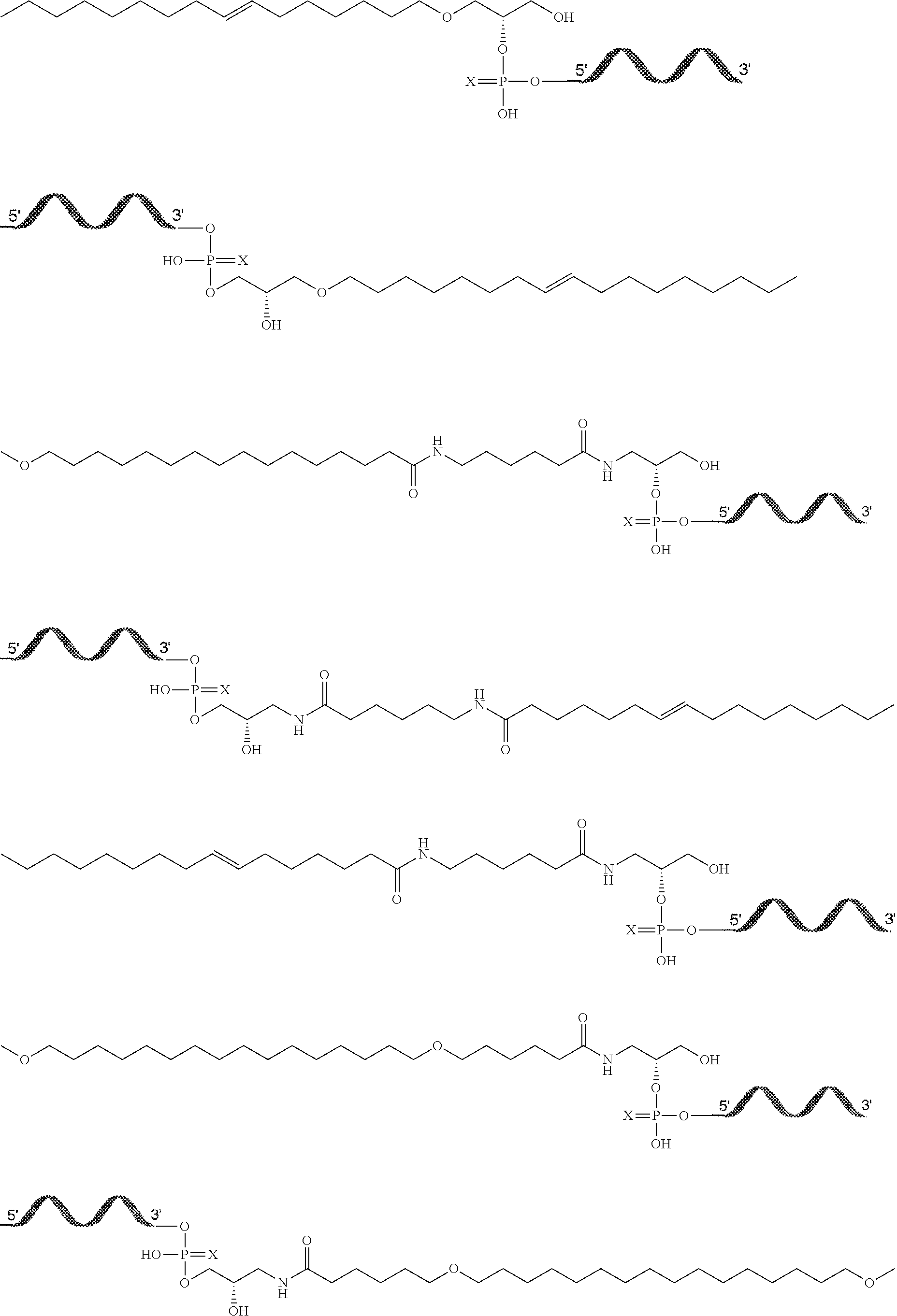 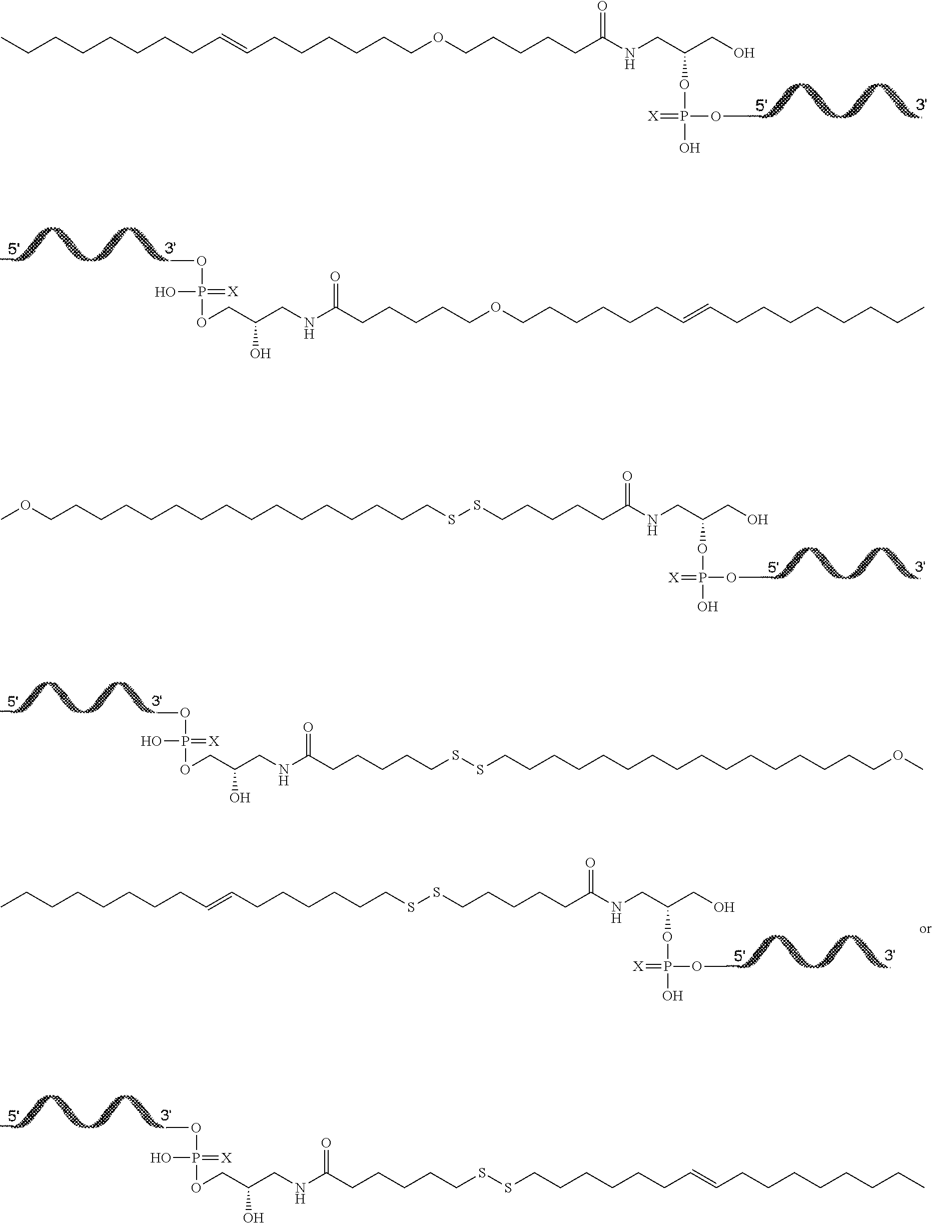
wherein,
X═O or S; and the ribbon represents the sense strand of siRNA or antisense oligonucleotide (ASO);
or wherein the sense strand of the siRNA duplex is modified at one or both ends with one or more moieties comprising:

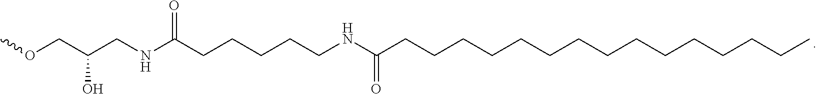

18.-35. (canceled)

36. A pharmaceutical composition comprising the RNAi agent of claim 1 in combination or association with a pharmaceutically accepted diluent or carrier.

37. A method of treating a disease or condition selected from inflammatory bowel disease (IBD), Crohn's disease (CD), ulcerative colitis (UC), susceptibility to bacterial infections such as Hansen's disease/leprosy, and/or Parkinson's disease (PD), comprising administering an effective amount of the RNAi agent according to claim 1 to a patient in need thereof.

38. (canceled)

39. A method of treating a disease or condition associated with LRRK2 gene expression comprising administering an effective amount of the RNAi agent according to claim 1 to a patient in need thereof.

40. The method of claim 39, wherein the disease or condition is characterized by overactivity or overexpression of Lrrk2 or an aberrant form of Lrrk2, optionally wherein the disease or condition is associated with a mutation of LRRK2.

41. (canceled)

42. The method of claim 39, wherein the disease or condition is selected from the group consisting of inflammatory bowel disease (IBD), Crohn's disease (CD), ulcerative colitis (UC), susceptibility to bacterial infections such as Hansen's disease/leprosy, and Parkinson's disease (PD), optionally wherein the disease or condition is Parkinson's disease.

43. (canceled)

44. The method of claim 40, wherein the disease or condition is associated with a LRRK2 mutation at one or more of positions 1371, 1437, 1441, 1699, 2019, and/or 2020, e.g. selected from I1371V, N1437H, N1437D, R1441C, R1441G, R1441H, R1441S, Y1699C, G2019S, and I2020T.

45. (canceled)

46. (canceled)

47. (canceled)

48. The RNAi agent of claim 1, wherein the the 5′-end of AS strand includes the following nucleosides: 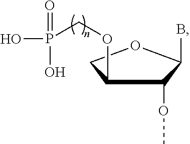
wherein n=1-4, and B is a nucleoside base.

49. The method of claim 39, wherein the RNAi agent is administered to the subject at a dose of about 0.01 mg/kg to about 50 mg/kg.

50. The method of claim 39, wherein the RNAi agent is administered to the subject intrathecally.

51. The method of claim 39, further comprising administering to the subject one or more additional therapeutic agents.

52.-61. (canceled)

62. A RNAi agent reducing LRRK2 expression, wherein the RNAi is selected from the group consisting of N3T130138-01, N3T130146-01, N3T130148-01, N3T130148-01, N3T131128-01, and N3T120059-01.
